(19) United States
(12) Patent Application Publication
(10) Pub. No.: US 2026/0199300 A1
(43) Pub. Date: Jul. 16, 2026

(54) PYRIDINE DERIVATIVES AS PROTEIN KINASE INHIBITORS

(71) Applicant: B.C.I. PHARMA, Liège (BE)

(72) Inventors: Rémi GUILLON, Liège (BE); Dominique SURLERAUX, Liège (BE); Claire AMIABLE, Liège (BE); Céline LASCHET, Liège (BE)

(21) Appl. No.: 19/132,489

(22) PCT Filed: Nov. 23, 2023
(86) PCT No.: PCT/EP2023/082907

§ 371 Date: May 23, 2025

(30) Foreign Application Priority Data
Nov. 24, 2022 (EP) 22209497.1

(51) Int. Cl. A61K 31/437; A61K 31/4439; A61K 31/444; A61K 31/5377; C07D 213/65; C07D 401/04; C07D 401/12; C07D 401/14; C07D 471/04

(52) U.S. Cl. CPC A61K 31/437; A61K 31/4439; A61K 31/444; A61K 31/5377; C07D 213/65; C07D 401/04; C07D 401/12; C07D 401/14; C07D 471/04

(57) ABSTRACT

The present disclosure relates to a compound suitable for use as a kinase inhibitor.

### FIELD OF THE INVENTION

The present invention is in the field of medicinal chemistry and pharmaceuticals.

### BACKGROUND OF THE INVENTION

Protein phosphorylation is the most common form of reversible post-translational modification, with an estimated 50% of all proteins undergoing phosphorylation. The phosphorylation state of any given protein is controlled by the coordinated action of specific kinases and phosphatases that add and remove phosphate, respectively. Particularly, protein kinases are a kind of protein phosphotransferases bringing the phosphate of ATP to the specific amino acid residue. They may conventionally be divided into five classes: tyrosine protein kinases, serine/threonine protein kinases, histidine protein kinases, tryptophan protein kinases and aspartyl/glutamoyl protein kinases.

Signaling networks that employ phosphorylation to modulate target activities have been shown to be critically involved in all aspects of cellular function, the abnormal activation of protein phosphorylation is frequently either a driver or direct consequence of the disease. Kinase signaling pathway dysregulation is associated with cancer, inflammatory disease, cardiovascular disease, neurodegenerative disease, and metabolic disease, through the constitutive activation of many downstream pathways, such as phosphatidyl-inositol 3-kinase/v-akt murine thymoma viral oncogene homolog 1 (PIK3/AKT), mitogen-activated protein kinase/extracellular signal regulated kinase (MAPK/ERK) and signal transducer and activator of transcription 5 (STAT5). Consequently, protein kinases represent important therapeutic targets.

In tumours, the abnormal oncogenic activation of protein kinases derives from multiple types of genetic and epigenetic changes. These alterations result in increased specific activity of the kinase itself, its overexpression, or the loss of negative regulation leading to uncontrolled cellular growth and sustained malignant behaviour. The signalling networks operating in cancer cells can also contribute to innate or acquired resistance to treatment, since they are able to create the most common or rare oncogenic mutations different from tumour to tumour. Hence, the search for small-molecule inhibitors targeting the altered protein kinase molecules in tumour cells has become a major research focus in the academia and pharmaceutical companies.

Such inhibitors can be products that are derived (isolated) from sources such as plants, animals or microorganisms, or can be small-molecules that are designed (synthetized).

WO 2004/022572 discloses classes of biologically active compounds interacting with kinases, and the preparation of these compounds.

In cancerology, there are currently multiple examples of small molecule kinase inhibitors with both selectivity and suitable pharmaceutical properties that have produced meaningful clinical benefit. For instance, pexidartinib is utilized to inhibit the colony-stimulating factor-1 receptor (CSF1R), the KIT proto-oncogene receptor tyrosine kinase (KIT) and the FMS-like tyrosine kinase 3 (FLT3) in, for example, the treatments of patients with symptomatic tenosynovial giant cell tumors (TGCT); edicotinib to inhibit the CSF1R and currently in phase II for acute myeloid leukemia, cognition disorders or Crohn's disease; or nintedanib to inhibit the endothelial growth factor receptor (VEGFR), fibroplast growth factor receptor (FGFR), platelet-derived growth factor receptor (PDGFR) and CSF1R in, for example, the treatment of idiopathic pulmonary fibrosis.

There is still a great need to develop potent inhibitors of protein kinase that are useful in treating the various protein kinase-related conditions.

In this sense, WO 2011/090738 A2 discloses compounds that are able to inhibit B-RAF and B-RAF mutations and methods for treating diseases related to B-RAF and B-RAF mutation modulation.

US 2009/0325945 describes active compounds, specifically, certain imidazo[4,5-b]pyridin-2-one and oxazolo[4,5-b]pyridin-2-one compounds and analogs inhibiting RAF (e.g., B-RAF) activity in a cell, in vitro or in vivo, inhibiting receptor tyrosine kinase (RTK) activity, such as FGFR, Tie, VEGFR and/or Eph activity, for example, FGFR-1, FGFR-2, FGFR-3, Tie2, VEGFR-2 and/or EphB2 activity, in a cell, in vitro or in vivo.

US 2015/0182526: This document describes therapeutic compounds for treating proliferative disorders, cancer, etc., and more specifically certain pyrido[2,3-b]pyrazin-8-substituted compounds, which, inter alia, inhibit RAF (e.g., B-RAF) activity and inhibit receptor tyrosine kinase (RTK) activity.

However, despite the growing effort in developing new protein kinase inhibitors based therapies, there is still a need for protein kinase inhibitors which may overcome the disadvantages of current protein kinase therapies such as side effects, limited efficacy, the emerging of resistance, and compliance failures.

### SUMMARY OF THE INVENTION

The inventors have surprisingly found that the use of protein kinase inhibitors according to the invention allows to provide an improved treatment of dysregulated protein kinase related diseases, by developing a therapy that is more effective, that reduces side effects, that limits the emerging of resistance and that facilitates compliance.

Therefore, the present invention provides a compound suitable for use as a protein kinase inhibitor according to any one of formulae (I) to (VII) [compound (C) hereinafter], or the N-oxide, pharmaceutically acceptable salt, pharmaceutically acceptable solvate, or stereoisomer thereof,

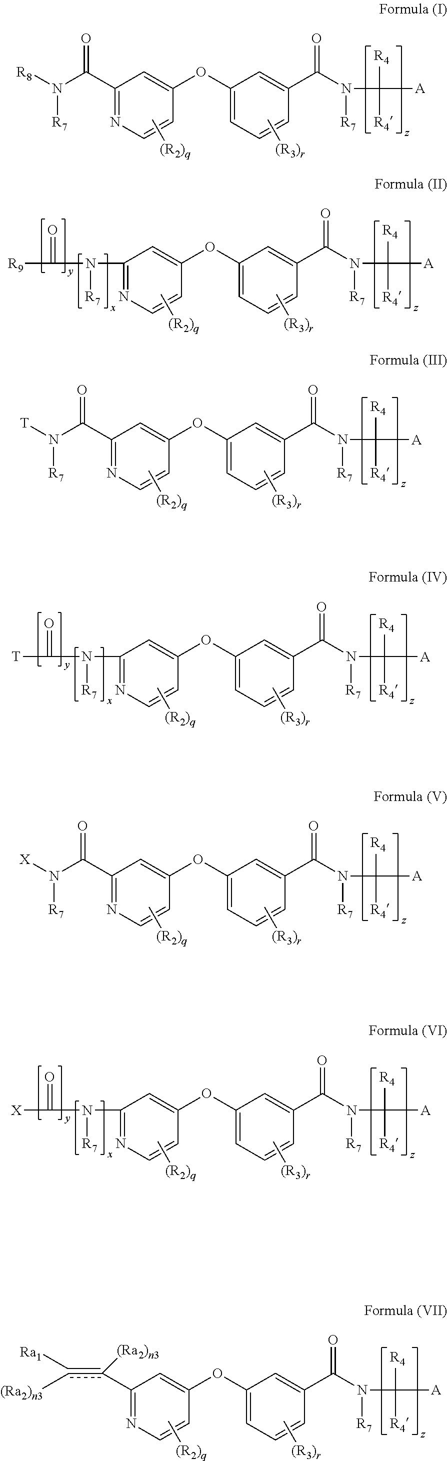

wherein:
each of A is independently selected from the group consisting of cycloalkyl, heterocyclyl, aryl, heteroaryl, and aralkyl, wherein said cycloalkyl, heterocyclyl, aryl, heteroaryl, and aralkyl are optionally substituted with one or more substituents independently selected from the group consisting of halo, NO₂, C₁₋₆ alkyl, C₂₋₄ alkenyl, C₂₋₄ alkynyl, cycloalkyl, heterocyclyl, aryl, heteroaryl, CF₃, CN, OR₁₁, SR₁₁, N(R₁₁)₂, OC(R₁₁)₂O, OC(R₁₁)₂C(R₁₁)₂O, S(O)R₁₂, SO₂R₁₂, SO₂N(R₁₁)₂, S(O)₃R₁₁, P(═O)(OR₁₁)₂, P(═O)(R₁₁)₂ NR₁₁COR₁₂, COR₁₁, C(O)OR₁₁, CON(R₁₁)₂, OC(O)R₁₁, and OCON(R₁₁)₂, and each optional alkyl, alkenyl, alkynyl, cycloalkyl, aryl, heterocyclyl, and heteroaryl substituents is further optionally substituted with halo, NO₂, C₁₋₆ alkyl, cycloalkyl, aryl, CF₃, N(R₁₁)₂, COR₁₁, CON(R₁₁)₂, OC(O)R₁₁, CN, or
OR₁₁; and wherein each of R₁₁ and R₁₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, C₁₋₆ alkyl or aryl or heteroaryl amide, OR₃₁ or N(R₃₂)₂, wherein each of R₃₁ and R₃₂, independently from each other and at each occurrence, is selected from the group consisting of hydrogen and C₁₋₄ alkyl.
each of R₄ and R′₄, independently from each other and at each occurrence, are selected from hydrogen or C₁₋₆ alkyl, and z is an integer in the range from 0 to 2; with the proviso that when z=0, then A and R₇ may form together a saturated or unsaturated cyclic moiety;
each of R₇, independently from each other and at each occurrence is selected from hydrogen, C₁₋₆ alkyl, cycloalkyl, wherein said alkyl and cycloalkyl are optionally substituted by a halogen atom, CF₃, N(R₁₁)₂, CN, or OR₁₁; and wherein each of R₁₁, independently from each other and at each occurrence, is selected from the group consisting of hydrogen, C₁₋₆ alkyl, and CF₃;
each of R₃, independently from each other and at each occurrence, is selected from the group consisting of hydrogen, halo, C₁₋₆ alkyl, C₂₋₆ alkenyl, C₂₋₆ alkynyl, cycloalkyl, heterocyclyl, aryl, heteroaryl, aralkyl, CF₃, CN, OR₂₁, SR₂₁, N(R₂₁)₂, NC(O)R₂₁, NCON(R₂₁)₂, COR₂₁, C(O)OR₂₁, CON(R₂₁)₂, OC(O)R₂₁, OCON(R₂₁)₂, OC(R₂₁)₂O, and OC(R₂₁)₂C(R₂₂)₂O, wherein said alkyl, alkenyl, alkynyl, cycloalkyl, heterocyclyl, aryl, heteroaryl and aralkyl are optionally substituted with one or more substituents selected from halo, C₁₋₆ alkyl, CF₃, N(R₂₁)₂, CN, or OR₂₁; and wherein each of R₂₁ and R₂₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, and aralkyl, and wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, aryl, OR₃₁ or N(R₃₂)₂, wherein each of R₃₁ and R₃₂, independently from each other and at each occurrence, is selected from the group consisting of hydrogen and C₁₋₄ alkyl; each of r is an integer in the range from 0 to 3; with the proviso that when R₃═NR₂₁, and R₇═H, then R₃ and NR₇ may form together a saturated or unsaturated cyclic moiety;
each of R₂, independently from each other and at each occurrence, is selected from the group consisting of hydrogen, halo, C₁₋₆ alkyl, C₂₋₆ alkenyl, C₂₋₆ alkynyl, cycloalkyl, heterocyclyl, aryl, and heteroaryl, CF₃, CN, NO₂, OR₂₁, SR₂₁, N(R₂₁)₂, COR₂₁, C(O)OR₂₁, CON(R₂₁)₂, OC(O)R₂₁, OCON(R₂₁)₂, NC(O)R₂₁, NCON(R₂₁)₂, OC(R₂₁)₂O and OC(R₂₁)₂C(R₂₂)₂O, wherein said alkyl, alkenyl, alkynyl, cycloalkyl, heterocyclyl, aryl, and heteroaryl are optionally substituted with one or more substituents selected from halo, C₁₋₆ alkyl, C₂₋₆ alkenyl, C₂₋₆ alkynyl, cycloalkyl, heterocyclyl, CF₃, COR₂₁, CON(R₂₁)₂, C(O)OR₂₁, N(R₂₁)₂, CN, or OR₂₁, and each optional alkyl, alkenyl, alkynyl, cycloalkyl, heterocyclyl, aryl or heteroaryl substituent is further optionally substituted with heterocyclyl, N(R₁₁)₂, or OR₁₁; and wherein each of R₂₁ and R₂₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, and aralkyl, and wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, aryl, OR₃₁ or N(R₃₂)₂, wherein each of R₃₁ and R₃₂, independently from each other and at each occurrence, is selected from the group consisting of hydrogen and C₁₋₄ alkyl; each of q is an integer in the range from 0 to 2;
each of x and y are independently integers equal to 0 or 1;
R₈ is independently selected from the group consisting of C₆₋₁₂ alkyl, C₂₋₆ alkenyl, C₂₋₆ alkynyl, cycloalkyl and heterocyclyl, wherein said alkyl, alkenyl, alkynyl, cycloalkyl, and heterocyclyl are optionally substituted by a halogen atom, an aryl group, an aralkyl group, CF₃, N(R₁₁)₂, CN, or OR₁₁; and wherein each of R₁₁, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, alkyl or aryl or heteroaryl amide, OR₃₁ or N(R₃₂)₂, wherein each of R₃₁ and R₃₂, independently from each other and at each occurrence, is selected from the group consisting of hydrogen and C₁₋₄ alkyl;
R₉ is selected from the group consisting of hydrogen, C₁₋₆ alkyl, C₂₋₆ alkenyl, C₂₋₆ alkynyl, cycloalkyl, heterocyclyl, N(R₁₁)₂ and CN, wherein said alkyl, alkenyl, alkynyl, cycloalkyl, and heterocyclyl are optionally substituted by a halogen atom, an aryl group, an aralkyl group, an heterocyclyl group, CF₃, N(R₁₁)₂, CN, or OR₁₁; and wherein each of R₁₁, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, heteroaryl optionally substituted with a C₁₋₄ alkyl, aryl, heteroaryl, and aralkyl substituents are optionally substituted with halo, C₁₋₆ alkyl, cycloalkyl, heterocyclyl, alkyl or aryl or heteroaryl amide, OR₃₁ or N(R₃₂)₂, wherein each of R₃₁ and R₃₂, independently from each other and at each occurrence, is selected from the group consisting of hydrogen and C₁₋₄ alkyl, with the proviso that if x=1 and y=0, R₉ is different from heterocyclyl, and from C₁₋₆ alkyl wherein said alkyl is optionally substituted with heterocyclyl; and with the proviso that if x=0 and y=0, R₉ is different from hydrogen, and C₁₋₆ alkyl, wherein said alkyl is optionally substituted with heterocyclyl and N(R₁₁)₂; with the proviso that when x=0 and y=0, R₉ and R₂ may form together a saturated or an unsaturated cyclic moiety; with the proviso that when x=0 and y=0 and when R₉ and R₂ form together a saturated or an unsaturated cyclic moiety, R₉ is NR₁₁; with the proviso that when x=1 and y=1, R₉ is different from N(R₁₁)₂; and with the proviso that when x=0, y=0 and z=0, R₉ is different from pyrrole.
each of T is independently the moiety of formula (T-a) herein below:

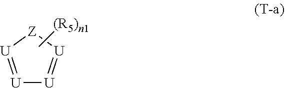

wherein:
each of U, independently from each other and at each occurrence, is selected from the group consisting of C, C-halo, C—R, and N; wherein R is selected from hydrogen, OR₁₁, N(R₁₁)₂, a C₁₋₆ alkyl or a cycloalkyl which are optionally substituted by a halogen atom, an aryl group or an aralkyl group, wherein each of R₁₁, independently from each other and at each occurrence, is selected from the group consisting of hydrogen or C₁₋₄ alkyl; with the proviso that at least one U is different from N;
each of Z, independently from each other and at each occurrence is selected from C(R)₂, O, S and NR₇, wherein R, independently from each other and at each occurrence is selected from hydrogen or an C₁₋₆ alkyl which is optionally substituted by a halogen atom, an aryl group or an aralkyl group, wherein R₇ is selected from the group consisting of hydrogen, C₁₋₆ alkyl, C₁₋₆ alkenyl, cycloalkyl, heterocyclyl, aryl, aralkyl and CF₃;
each of R₅, independently from each other and at each occurrence is selected from the group consisting of C₁₋₆ alkyl, C₂₋₆ alkenyl, C₂₋₆ alkynyl, cycloalkyl, heterocyclyl, aryl, heteroaryl, aralkyl, halo, CF₃, OR₁₁, SR₁₁, N(R₁₁)₂, COOR₁₁, CO(R₁₁)₂, CON(R₁₁)₂, and each optional alkyl, alkenyl, alkynyl, cycloalkyl, aryl, heterocyclyl, heteroaryl, aralkyl substituent is further optionally substituted with halo, C₁₋₆ alkyl, cycloalkyl, aryl, heterocyclyl, N(R₁₁)₂, CN, OR₁₁, C(═O)OR₁₁, P(═O)(OR₁₁)₂, P(═O)(R₁₁)₂, CN or CF₃ and wherein each of R₁₁, independently from each other and at each occurrence, is selected from the group consisting of hydrogen, C₁₋₆ alkyl, cycloalkyl, and heterocyclyl; each of n1 is an integer in the range from 0 to 2;
each of X is independently the moiety of formula (X-a) herein below:

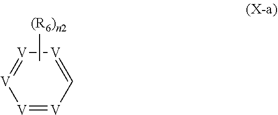

wherein:
each of V, independently from each other and at each occurrence, is selected from the group consisting of C, C-halo, C—R, and N; wherein R is selected from hydrogen, OR₁₁, N(R₁₁)₂, a C₁₋₆ alkyl or a cycloalkyl which are optionally substituted by a halogen atom, an aryl group or an aralkyl group, wherein each of R₁₁, independently from each other and at each occurrence, is selected from hydrogen or C₁₋₄ alkyl;
each of R₆, independently from each other and at each occurrence is selected from the group consisting of C₁₋₆ alkyl, C₂₋₆ alkenyl, C₂₋₆ alkynyl, cycloalkyl, heterocyclyl, aryl, heteroaryl, aralkyl, halo, CF₃, OR₁₁, SR₁₁, N(R₁₁)₂, COOR₁₁, CO(R₁₁)₂, CON(R₁₁)₂, and each optional alkyl, alkenyl, alkynyl, cycloalkyl, aryl, heterocyclyl, heteroaryl, aralkyl substituent is further optionally substituted with halo, C₁₋₆ alkyl, cycloalkyl, aryl, heterocyclyl, N(R₁₁)₂, CN, OR₁₁, C(═O)OR₁₁, P(═O)(OR₁₁)₂, P(═O)(R₁₁)₂, CN or CF₃ and wherein each of R₁₁, independently from each other and at each occurrence, is selected from the group consisting of hydrogen, C₁₋₆ alkyl, cycloalkyl, and heterocyclyl; each of n2 is an integer in the range from 0 to 4;
the dash bond represents an optional triple bond;
Rₐ₁ is independently selected from the group consisting of hydrogen, C₁₋₆ alkyl, cycloalkyl, heterocyclyl, aryl, heteroaryl, aralkyl, CF₃, CN, OR₁₁, SR₁₁, N(R₁₁)₂, COR₁₁, C(O)OR₁₁, wherein said alkyl cycloalkyl, heterocyclyl, aryl, heteroaryl and aralkyl are optionally substituted by halo, NO₂, C₁₋₆ alkyl, C₂₋₆ alkenyl, C₂₋₆ alkynyl, cycloalkyl, heterocyclyl, phenyl, heteroaryl, CF₃, CN, OR₁₁, SR₁₁, N(R₁₁)₂, COR₁₁, and C(O)OR₁₁, and each optional alkyl, alkenyl, cycloalkyl, phenyl, heterocyclyl, heteroaryl substituent is further optionally substituted with halo, NO₂, C₁₋₆ alkyl, cycloalkyl, phenyl, N(R₁₁)₂, CN, or OR₁₁; and wherein each of R₁₁ is selected from the group consisting of hydrogen, C₁₋₆ alkyl, cycloalkyl, heterocyclyl, phenyl, heteroaryl, and aralkyl, wherein said alkyl, cycloalkyl, heterocyclyl, phenyl, heteroaryl, and aralkyl substituents are optionally substituted with halo, C₁₋₆ alkyl, cycloalkyl or heterocyclyl.
each of Rₐ₂, independently from each other and at each occurrence, is selected from the group consisting of hydrogen, C₁₋₆ alkyl, cycloalkyl, heterocyclyl, aryl, heteroaryl, aralkyl, CF₃, CN, OR₁₁, SR₁₁, N(R₁₁)₂, COR₁₁, C(O)OR₁₁, wherein said alkyl, alkenyl, alkynyl, cycloalkyl, heterocyclyl, aryl, heteroaryl and aralkyl are optionally substituted by halo, NO₂, C₁₋₆ alkyl, C₂₋₆ alkenyl, C₂₋₆ alkynyl, cycloalkyl, heterocyclyl, phenyl, heteroaryl, CF₃, CN, OR₁₁, SR₁₁, N(R₁₁)₂, COR₁₁, and C(O)OR₁₁, and each optional alkyl, alkenyl, cycloalkyl, phenyl, heterocyclyl, heteroaryl substituent is further optionally substituted with halo, NO₂, C₁₋₆ alkyl, cycloalkyl, phenyl, N(R₁₁)₂, CN, or OR₁₁; and wherein each of R₁₁ is selected from the group consisting of hydrogen, C₁₋₆ alkyl, cycloalkyl, heterocyclyl, phenyl, heteroaryl, and aralkyl, wherein said alkyl, cycloalkyl, heterocyclyl, phenyl, heteroaryl, and aralkyl substituents are optionally substituted with halo, C₁₋₆ alkyl, cycloalkyl or heterocyclyl; and wherein n3 is an integer equal to 0 or 1; with the proviso that when the dash bond represents a triple bond, n3 is 0;
   wherein said cycloalkyl is a monocyclic, bicyclic or tricyclic ring system of 3-6 ring members per ring; said heterocyclyl is a saturated, partially saturated or completely saturated monocycle, bicycle or tricycle containing 3 to 12 carbon atoms and 1 or 2 heteroatoms independently selected from O or N; said aryl is phenyl, naphthyl or anthracenyl optionally carbocyclic fused with a cycloalkyl or heterocyclyl of 5-7 ring members; said heteroaryl is a monocyclic ring structure containing 5 or 6 ring atoms, or a bicyclic aromatic group having 8 to 10 atoms, containing 1-3 heteroatoms independently selected from O or N.

The present invention further relates to a pharmaceutical composition comprising a carrier, and as active ingredient an effective amount of a compound as defined in any one of the embodiments presented herein.

The present invention relates to a compound as defined in any one of the embodiments presented herein, for use as a medicament.

The present invention relates to a compound as defined in any one of the embodiments presented herein for use in the treatment of a disease selected from cancer, metabolic disorders (such as diabetes), inflammatory and autoimmune disorders (such as inflammatory bowel diseases, e.g. Crohn's disease and ulcerative colitis, inflammatory pulmonary diseases, rheumatoid arthritis, lupus nephritis, systemic lupus erythematosus and psoriasis and psoriasis arthritis), neurological disorders (such as Alzheimer's disease, Parkinson's disease, multiple sclerosis, Charcot-Marie-Tooth neuropathy, amyotrophic lateral sclerosis and epilepsy), atherosclerosis and cardiovascular diseases, Sjogren Syndrome, renal allograft rejection, viral induced diseases, circulatory diseases, bone osteolysis and osteoporosis, osteoarthritis, sarcopenia, Langerhans cell histiocytosis, spinal cord injury, endometriosis, asthma and allergic asthma, eye diseases (such as retinopathies, age-related macular degeneration and uveitis) chronic and neuropathic pain, and fibro-proliferative diseases.

The present invention relates to a compound as defined in any one of the embodiments presented herein, for use in the treatment of pain sensitization.

The present invention further relates to a method of inhibiting protein kinase activity in a warm-blooded animal said method comprising the administration to an animal in need thereof, of a kinase-inhibitory effective amount of a compound according to any one of the embodiments presented herein.

The present invention further relates to a method of treating a disease selected from cancer, metabolic disorders (such as diabetes), inflammatory and autoimmune disorders (such as inflammatory bowel diseases, e.g. Crohn's disease and ulcerative colitis, inflammatory pulmonary diseases, rheumatoid arthritis, lupus nephritis, systemic lupus erythematosus and psoriasis and psoriasis arthritis), neurological disorders (such as Alzheimer's disease, Parkinson's disease, multiple sclerosis, Charcot-Marie-Tooth neuropathy, amyotrophic lateral sclerosis and epilepsy), atherosclerosis and cardiovascular diseases, Sjogren Syndrome, renal allograft rejection, viral induced diseases, circulatory diseases, bone osteolysis and osteoporosis, osteoarthritis, sarcopenia, Langerhans cell histiocytosis, spinal cord injury, endometriosis, asthma and allergic asthma, eye diseases (such as retinopathies, age-related macular degeneration and uveitis) chronic and neuropathic pain, and fibro-proliferative diseases in a warm-blooded animal said method comprising the administration to an animal in need thereof of an effective amount of a compound according to any one of the embodiments presented herein.

### DETAILED DESCRIPTION OF THE INVENTION

A first aspect of the present invention relates to a compound suitable for use as a protein kinase inhibitor according to any one of formulae (I) to (VII) [compound (C) hereinafter], or the N-oxide, pharmaceutically acceptable salt, pharmaceutically acceptable solvate, or stereoisomer thereof,

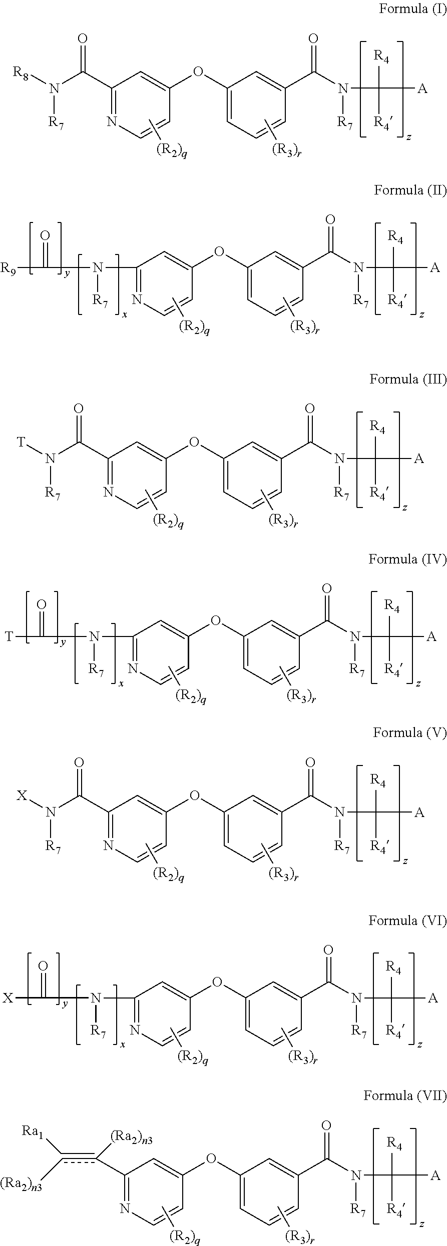

wherein:
each of A is independently selected from the group consisting of cycloalkyl, heterocyclyl, aryl, heteroaryl, and aralkyl, wherein said cycloalkyl, heterocyclyl, aryl, heteroaryl, and aralkyl are optionally substituted with one or more substituents independently selected from the group consisting of halo, NO₂, C₁₋₆ alkyl, C₂₋₄ alkenyl, C₂₋₄ alkynyl, cycloalkyl, heterocyclyl, aryl, heteroaryl, CF₃, CN, OR₁₁, SR₁₁, N(R₁₁)₂, OC(R₁₁)₂O, OC(R₁₁)₂C(R₁₁)₂O, S(O)R₁₂, SO₂R₁₂, SO₂N(R₁₁)₂, S(O)₃R₁₁, P(═O)(OR₁₁)₂, P(═O)(R₁₁)₂ NR₁₁COR₁₂, COR₁₁, C(O)OR₁₁, CON(R₁₁)₂, OC(O)R₁₁, and OCON(R₁₁)₂, and each optional alkyl, alkenyl, alkynyl, cycloalkyl, aryl, heterocyclyl, and heteroaryl substituents is further optionally substituted with halo, NO₂, C₁₋₆ alkyl, cycloalkyl, aryl, CF₃, N(R₁₁)₂, COR₁₁, CON(R₁₁)₂, OC(O)R₁₁, CN, or OR₁₁; and wherein each of R₁₁ and R₁₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, C₁₋₆ alkyl or aryl or heteroaryl amide, OR₃₁ or N(R₃₂)₂, wherein each of R₃₁ and R₃₂, independently from each other and at each occurrence, is selected from the group consisting of hydrogen and C₁₋₄ alkyl.
each of R₄ and R′₄, independently from each other and at each occurrence, are selected from hydrogen or C₁₋₆ alkyl, and z is an integer in the range from 0 to 2; with the proviso that when z=0, then A and R₇ may form together a saturated or unsaturated cyclic moiety;
each of R₇, independently from each other and at each occurrence is selected from hydrogen, C₁₋₆ alkyl, cycloalkyl, wherein said alkyl and cycloalkyl are optionally substituted by a halogen atom, CF₃, N(R₁₁)₂, CN, or OR₁₁; and wherein each of R₁₁, independently from each other and at each occurrence, is selected from the group consisting of hydrogen, C₁₋₆ alkyl, and CF₃;
each of R₃, independently from each other and at each occurrence, is selected from the group consisting of hydrogen, halo, C₁₋₆ alkyl, C₂₋₆ alkenyl, C₂₋₆ alkynyl, cycloalkyl, heterocyclyl, aryl, heteroaryl, aralkyl, CF₃, CN, OR₂₁, SR₂₁, N(R₂₁)₂, NC(O)R₂₁, NCON(R₂₁)₂, COR₂₁, C(O)OR₂₁, CON(R₂₁)₂, OC(O)R₂₁, OCON(R₂₁)₂, OC(R₂₁)₂O, and OC(R₂₁)₂C(R₂₂)₂O, wherein said alkyl, alkenyl, alkynyl, cycloalkyl, heterocyclyl, aryl, heteroaryl and aralkyl are optionally substituted with one or more substituents selected from halo, C₁₋₆ alkyl, CF₃, N(R₂₁)₂, CN, or OR₂₁; and wherein each of R₂₁ and R₂₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, and aralkyl, and wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, aryl, OR₃₁ or N(R₃₂)₂, wherein each of R₃₁ and R₃₂, independently from each other and at each occurrence, is selected from the group consisting of hydrogen and C₁₋₄ alkyl; each of r is an integer in the range from 0 to 3; with the proviso that when R₃═NR₂₁, and R₇═H, then R₃ and NR₇ may form together a saturated or unsaturated cyclic moiety;
each of R₂, independently from each other and at each occurrence, is selected from the group consisting of hydrogen, halo, C₁₋₆ alkyl, C₂₋₆ alkenyl, C₂₋₆ alkynyl, cycloalkyl, heterocyclyl, aryl, and heteroaryl, CF₃, CN, NO₂, OR₂₁, SR₂₁, N(R₂₁)₂, COR₂₁, C(O)OR₂₁, CON(R₂₁)₂, OC(O)R₂₁, OCON(R₂₁)₂, NC(O)R₂₁, NCON(R₂₁)₂, OC(R₂₁)₂O and OC(R₂₁)₂C(R₂₂)₂O, wherein said alkyl, alkenyl, alkynyl, cycloalkyl, heterocyclyl, aryl, and heteroaryl are optionally substituted with one or more substituents selected from halo, C₁₋₆ alkyl, C₂₋₆ alkenyl, C₂₋₆ alkynyl, cycloalkyl, heterocyclyl, CF₃, COR₂₁, CON(R₂₁)₂, C(O)OR₂₁, N(R₂₁)₂, CN, or OR₂₁, and each optional alkyl, alkenyl, alkynyl, cycloalkyl, heterocyclyl, aryl or heteroaryl substituent is further optionally substituted with heterocyclyl, N(R₁₁)₂, or OR₁₁; and wherein each of R₂₁ and R₂₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, and aralkyl, and wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, aryl, OR₃₁ or N(R₃₂)₂, wherein each of R₃₁ and R₃₂, independently from each other and at each occurrence, is selected from the group consisting of hydrogen and C₁₋₄ alkyl; each of q is an integer in the range from 0 to 2;
each of x and y are independently integers equal to 0 or 1;
R₈ is independently selected from the group consisting of C₆₋₁₂ alkyl, C₂₋₆ alkenyl, C₂₋₆ alkynyl, cycloalkyl and heterocyclyl, wherein said alkyl, alkenyl, alkynyl, cycloalkyl, and heterocyclyl are optionally substituted by a halogen atom, an aryl group, an aralkyl group, CF₃, N(R₁₁)₂, CN, or OR₁₁; and wherein each of R₁₁, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, alkyl or aryl or heteroaryl amide, OR₃₁ or N(R₃₂)₂, wherein each of R₃₁ and R₃₂, independently from each other and at each occurrence, is selected from the group consisting of hydrogen and C₁₋₄ alkyl;
R₉ is selected from the group consisting of hydrogen, C₁₋₆ alkyl, C₂₋₆ alkenyl, C₂₋₆ alkynyl, cycloalkyl, heterocyclyl, N(R₁₁)₂ and CN, wherein said alkyl, alkenyl, alkynyl, cycloalkyl, and heterocyclyl are optionally substituted by a halogen atom, an aryl group, an aralkyl group, an heterocyclyl group, CF₃, N(R₁₁)₂, CN, or OR₁₁; and wherein each of R₁₁, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, heteroaryl optionally substituted with a C₁₋₄ alkyl, aryl, heteroaryl, and aralkyl substituents are optionally substituted with halo, C₁₋₆ alkyl, cycloalkyl, heterocyclyl, alkyl or aryl or heteroaryl amide, OR₃₁ or N(R₃₂)₂, wherein each of R₃₁ and R₃₂, independently from each other and at each occurrence, is selected from the group consisting of hydrogen and C₁₋₄ alkyl, with the proviso that if x=1 and y=0, R₉ is different from heterocyclyl, and from C₁₋₆ alkyl wherein said alkyl is optionally substituted with heterocyclyl; and with the proviso that if x=0 and y=0, R₉ is different from hydrogen, and C₁₋₆ alkyl, wherein said alkyl is optionally substituted with heterocyclyl and N(R₁₁)₂; with the proviso that when x=0 and y=0, R₉ and R₂ may form together a saturated or an unsaturated cyclic moiety; with the proviso that when x=0 and y=0 and when R₉ and R₂ form together a saturated or an unsaturated cyclic moiety, R₉ is NR₁₁; with the proviso that when x=1 and y=1, R₉ is different from N(R₁₁)₂; and with the proviso that when x=0, y=0 and z=0, R₉ is different from pyrrole.
each of T is independently the moiety of formula (T-a) herein below:

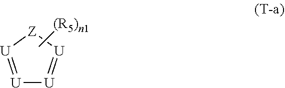

wherein:
each of U, independently from each other and at each occurrence, is selected from the group consisting of C, C-halo, C—R, and N; wherein R is selected from hydrogen, OR₁₁, N(R₁₁)₂, a C₁₋₆ alkyl or a cycloalkyl which are optionally substituted by a halogen atom, an aryl group or an aralkyl group, wherein each of R₁₁, independently from each other and at each occurrence, is selected from the group consisting of hydrogen or C₁₋₄ alkyl; with the proviso that at least one U is different from N;
each of Z, independently from each other and at each occurrence is selected from C(R)₂, O, S and NR₇, wherein R, independently from each other and at each occurrence is selected from hydrogen or an C₁₋₆ alkyl which is optionally substituted by a halogen atom, an aryl group or an aralkyl group, wherein R₇ is selected from the group consisting of hydrogen, C₁₋₆ alkyl, C₁₋₆ alkenyl, cycloalkyl, heterocyclyl, aryl, aralkyl and CF₃;
each of R₅, independently from each other and at each occurrence is selected from the group consisting of C₁₋₆ alkyl, C₂₋₆ alkenyl, C₂₋₆ alkynyl, cycloalkyl, heterocyclyl, aryl, heteroaryl, aralkyl, halo, CF₃, OR₁₁, SR₁₁, N(R₁₁)₂, COOR₁₁, CO(R₁₁)₂, CON(R₁₁)₂, and each optional alkyl, alkenyl, alkynyl, cycloalkyl, aryl, heterocyclyl, heteroaryl, aralkyl substituent is further optionally substituted with halo, C₁₋₆ alkyl, cycloalkyl, aryl, heterocyclyl, N(R₁₁)₂, CN, OR₁₁, C(═O)OR₁₁, P(═O)(OR₁₁)₂, P(═O)(R₁₁)₂, CN or CF₃ and wherein each of R₁₁, independently from each other and at each occurrence, is selected from the group consisting of hydrogen, C₁₋₆ alkyl, cycloalkyl, and heterocyclyl; each of n1 is an integer in the range from 0 to 2;
each of X is independently the moiety of formula (X-a) herein below:

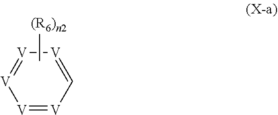

wherein:
each of V, independently from each other and at each occurrence, is selected from the group consisting of C, C-halo, C—R, and N; wherein R is selected from hydrogen, OR₁₁, N(R₁₁)₂, a C₁₋₆ alkyl or a cycloalkyl which are optionally substituted by a halogen atom, an aryl group or an aralkyl group, wherein each of R₁₁, independently from each other and at each occurrence, is selected from hydrogen or C₁₋₄ alkyl;
each of R₆, independently from each other and at each occurrence is selected from the group consisting of C₁₋₆ alkyl, C₂₋₆ alkenyl, C₂₋₆ alkynyl, cycloalkyl, heterocyclyl, aryl, heteroaryl, aralkyl, halo, CF₃, OR₁₁, SR₁₁, N(R₁₁)₂, COOR₁₁, CO(R₁₁)₂, CON(R₁₁)₂, and each optional alkyl, alkenyl, alkynyl, cycloalkyl, aryl, heterocyclyl, heteroaryl, aralkyl substituent is further optionally substituted with halo, C₁₋₆ alkyl, cycloalkyl, aryl, heterocyclyl, N(R₁₁)₂, CN, OR₁₁, C(═O)OR₁₁, P(═O)(OR₁₁)₂, P(═O)(R₁₁)₂, CN or CF₃ and wherein each of R₁₁, independently from each other and at each occurrence, is selected from the group consisting of hydrogen, C₁₋₆ alkyl, cycloalkyl, and heterocyclyl; each of n2 is an integer in the range from 0 to 4;
the dash bond represents an optional triple bond;
Rₐ₁ is independently selected from the group consisting of hydrogen, C₁₋₆ alkyl, cycloalkyl, heterocyclyl, aryl, heteroaryl, aralkyl, CF₃, CN, OR₁₁, SR₁₁, N(R₁₁)₂, COR₁₁, C(O)OR₁₁, wherein said alkyl cycloalkyl, heterocyclyl, aryl, heteroaryl and aralkyl are optionally substituted by halo, NO₂, C₁₋₆ alkyl, C₂₋₆ alkenyl, C₂₋₆ alkynyl, cycloalkyl, heterocyclyl, phenyl, heteroaryl, CF₃, CN, OR₁₁, SR₁₁, N(R₁₁)₂, COR₁₁, and C(O)OR₁₁, and each optional alkyl, alkenyl, cycloalkyl, phenyl, heterocyclyl, heteroaryl substituent is further optionally substituted with halo, NO₂, C₁₋₆ alkyl, cycloalkyl, phenyl, N(R₁₁)₂, CN, or OR₁₁; and wherein each of R₁₁ is selected from the group consisting of hydrogen, C₁₋₆ alkyl, cycloalkyl, heterocyclyl, phenyl, heteroaryl, and aralkyl, wherein said alkyl, cycloalkyl, heterocyclyl, phenyl, heteroaryl, and aralkyl substituents are optionally substituted with halo, C₁₋₆ alkyl, cycloalkyl or heterocyclyl.
each of Rₐ₂, independently from each other and at each occurrence, is selected from the group consisting of hydrogen, C₁₋₆ alkyl, cycloalkyl, heterocyclyl, aryl, heteroaryl, aralkyl, CF₃, CN, OR₁₁, SR₁₁, N(R₁₁)₂, COR₁₁, C(O)OR₁₁, wherein said alkyl, alkenyl, alkynyl, cycloalkyl, heterocyclyl, aryl, heteroaryl and aralkyl are optionally substituted by halo, NO₂, C₁₋₆ alkyl, C₂₋₆ alkenyl, C₂₋₆ alkynyl, cycloalkyl, heterocyclyl, phenyl, heteroaryl, CF₃, CN, OR₁₁, SR₁₁, N(R₁₁)₂, COR₁₁, and C(O)OR₁₁, and each optional alkyl, alkenyl, cycloalkyl, phenyl, heterocyclyl, heteroaryl substituent is further optionally substituted with halo, NO₂, C₁₋₆ alkyl, cycloalkyl, phenyl, N(R₁₁)₂, CN, or OR₁₁; and wherein each of R₁₁ is selected from the group consisting of hydrogen, C₁₋₆ alkyl, cycloalkyl, heterocyclyl, phenyl, heteroaryl, and aralkyl, wherein said alkyl, cycloalkyl, heterocyclyl, phenyl, heteroaryl, and aralkyl substituents are optionally substituted with halo, C₁₋₆ alkyl, cycloalkyl or heterocyclyl; and wherein n3 is an integer equal to 0 or 1; with the proviso that when the dash bond represents a triple bond, n3 is 0;
   wherein said cycloalkyl is a monocyclic, bicyclic or tricyclic ring system of 3-6 ring members per ring; said heterocyclyl is a saturated, partially saturated or completely saturated monocycle, bicycle or tricycle containing 3 to 12 carbon atoms and 1 or 2 heteroatoms independently selected from O or N; said aryl is phenyl, naphthyl or anthracenyl optionally carbocyclic fused with a cycloalkyl or heterocyclyl of 5-7 ring members; said heteroaryl is a monocyclic ring structure containing 5 or 6 ring atoms, or a bicyclic aromatic group having 8 to 10 atoms, containing 1-3 heteroatoms independently selected from O or N.

In a preferred embodiment of the present invention, A in compound (C) of formulae (I) to (VII) is independently selected from the group consisting of cycloalkyl, heterocyclyl, aryl, heteroaryl, and aralkyl, wherein said cycloalkyl, heterocyclyl, aryl, heteroaryl, and aralkyl are optionally substituted with one or more substituents independently selected from the group consisting of halo, NO₂, C₁₋₆ alkyl, C₂₋₄ alkenyl, C₂₋₄ alkynyl, cycloalkyl, heterocyclyl, aryl, heteroaryl, CF₃, CN, OR₁₁, SR₁₁, N(R₁₁)₂, OC(R₁₁)₂O, OC(R₁₁)₂C(R₁₁)₂O, P(═O)(OR₁₁)₂, P(═O)(R₁₁)₂ NR₁₁COR₁₂, COR₁₁, C(O)OR₁₁, CON(R₁₁)₂, OC(O)R₁₁, and OCON(R₁₁)₂, and each optional alkyl, alkenyl, alkynyl, cycloalkyl, aryl, heterocyclyl, heteroaryl substituent is further optionally substituted with halo, NO₂, C₁₋₆ alkyl, cycloalkyl, aryl, CF₃, N(R₁₁)₂, CN, or OR₁₁; and wherein each of R₁₁ and R₁₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, aryl, heteroaryl, and aralkyl substituents are optionally substituted with halo, C₁₋₆ alkyl, cycloalkyl, or heterocyclyl. More preferably, A is independently selected from the group consisting of cycloalkyl, heterocyclyl, aryl, heteroaryl, and aralkyl, wherein said cycloalkyl, heterocyclyl, aryl, heteroaryl, and aralkyl are optionally substituted with one or more substituents independently selected from the group consisting of halo, C₁₋₆ alkyl, cycloalkyl, heterocyclyl, aryl, heteroaryl, CF₃, CN, OR₁₁, N(R₁₁)₂, OC(R₁₁)₂O, OC(R₁₁)₂C(R₁₁)₂O, P(═O)(R₁₁)₂, COR₁₁, C(O)OR₁₁, CON(R₁₁)₂, OC(O)R₁₁, and each optional alkyl, alkenyl, alkynyl, cycloalkyl, aryl, heterocyclyl, heteroaryl substituent is further optionally substituted with C₁₋₄ alkyl or cycloalkyl; and wherein each of R₁₁ and R₁₂, independently from each other and at each occurrence, is selected from the group consisting of hydrogen, C₁₋₆ alkyl, cycloalkyl, heterocyclyl, aryl, heteroaryl, aralkyl and CF₃. More preferably, A is independently selected from the group consisting of cycloalkyl, heterocyclyl, aryl, heteroaryl, and aralkyl, wherein said cycloalkyl, heterocyclyl, aryl, heteroaryl, and aralkyl are optionally substituted with one or more substituents independently selected from the group consisting of halo, C₁₋₆ alkyl, CF₃, CN, OR₁₁, and P(═O) (R₁₁) 2; and wherein each of R₁₁, at each occurrence, is hydrogen or C₁₋₄ alkyl such as methyl, ethyl, propyl, isopropyl, butyl, tert-butyl, and isobutyl.

In one embodiment of the present invention, A in compound (C) of formulae (I) to (VII) is independently selected from the following moieties:

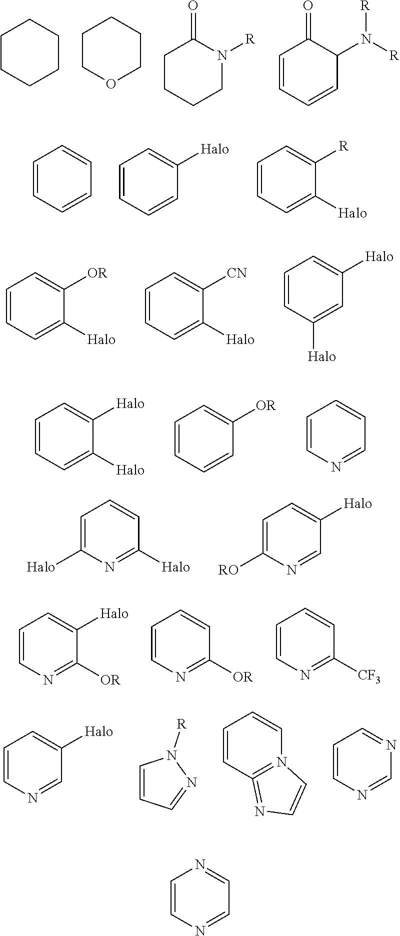

wherein each of halo is F, Cl, Br or I, and each of R is hydrogen or C₁₋₄ alkyl such as methyl, ethyl, propyl, isopropyl, butyl, tert-butyl, and isobutyl, preferably R is hydrogen, methyl, ethyl, 2-methylpropyl or tert-butyl.

In a preferred embodiment of the present invention, each of R₄ in compound (C) of formulae (I) to (VII) is hydrogen or C₁₋₄ alkyl such as methyl, ethyl, propyl, isopropyl, butyl, tert-butyl, isobutyl and the like. Even more preferably, R₄ is hydrogen or methyl.

In a preferred embodiment of to the present invention, each of R₄′ in compound (C) of formulae (I) to (VII) is hydrogen or C₁₋₄ alkyl such as methyl, ethyl, propyl, isopropyl, butyl, tert-butyl, isobutyl and the like. Even more preferably, R₄′ is hydrogen.

In a preferred embodiment of to the present invention, z in compound (C) of formulae (I) to (VII) is an integer equal to 0 or 1. Even more preferably, z is 1.

In a preferred embodiment of the present invention, each of R₇ in compound (C) of formulae (I) to (VII), independently from each other and at each occurrence, is hydrogen or C₁₋₄ alkyl such as methyl, ethyl, propyl, isopropyl, butyl, tert-butyl, isobutyl and the like. More preferably, each of R₇ independently from each other and at each occurrence is hydrogen or methyl. Even more preferably, each of R₇ independently from each other and at each occurrence is hydrogen.

In a preferred embodiment of the present invention, each of R₃ in compound (C) of formulae (I) to (VII), independently from each other and at each occurrence, is selected from the group consisting of hydrogen, halo, C₁₋₆ alkyl, cycloalkyl, heterocyclyl, CF₃, CN, OR₂₁, and N(R₂₁)₂, wherein said alkyl, cycloalkyl and heterocyclyl, are optionally substituted with one or more substituents selected from halo, C₁₋₆ alkyl, CF₃, N(R₂₁)₂, CN, or OR₂₁; and wherein each of R₂₁, independently from each other and at each occurrence, is selected from the group consisting of hydrogen, C₁₋₆ alkyl, C₃₋₆ cycloalkyl, heterocyclyl, aryl, heteroaryl, and aralkyl, and wherein said alkyl, cycloalkyl, heterocyclyl, aryl, heteroaryl, and aralkyl substituents are optionally substituted with halo, C₁₋₆ alkyl, cycloalkyl, heterocyclyl, or aryl. Preferably, R₃ is independently selected from the group consisting of hydrogen, halo, C₁₋₆ alkyl, cycloalkyl, CF₃, CN, OR₂₁, and N(R₂₁)₂, wherein said alkyl, and cycloalkyl, are optionally substituted with one or more substituents selected from halo, C₁₋₆ alkyl, CF₃, N(R₂₁)₂, CN, or OR₂₁; and wherein each of R₂₁, independently from each other and at each occurrence, is selected from the group consisting of hydrogen, and C₁₋₄ alkyl such as methyl, ethyl, propyl, isopropyl, butyl, tert-butyl, and isobutyl. More preferably, R₃ is independently selected from the group consisting of hydrogen, halo, and C₁₋₄ alkyl such as methyl, ethyl, propyl, isopropyl, butyl, tert-butyl, isobutyl, CF₃, CN, OR₂₁, and N(R₂₁)₂, and wherein each of R₂₁, independently from each other and at each occurrence, is selected from the group consisting of hydrogen, and C₁₋₄ alkyl. More preferably, R₃ is independently selected from the group consisting of hydrogen, halo, OC₁₋₄ alkyl, and C₁₋₄ alkyl such as methyl, ethyl, propyl, isopropyl, butyl, tert-butyl, and isobutyl. Even more preferably, R₃ is independently chosen from the group consisting of hydrogen, halo, OCH₃ and methyl.

In a preferred embodiment of the present invention, each of r in compound (C) of formulae (I) to (VII) is an integer equal to 0, 1 or 2. More preferably, each of r is an integer equal to 0 or 1. Even more preferably, each of r is an integer equal to 1.

In a preferred embodiment of the present invention, each of R₂ in compound (C) of formulae (I) to (VII) independently from each other and at each occurrence, is selected from the group consisting of hydrogen, halo, C₁₋₆ alkyl, cycloalkyl, heterocyclyl, aryl, and heteroaryl, CN, OR₂₁, and N(R₂₁)₂, wherein said alkyl, cycloalkyl, heterocyclyl, aryl, and heteroaryl are optionally substituted with one or more substituents selected from halo, C₁₋₆ alkyl, cycloalkyl, N(R₂₁)₂, CN, or OR₂₁; wherein R₂₁, independently from each other and at each occurrence, is selected from the group consisting of hydrogen, C₁₋₆ alkyl, cycloalkyl, heterocyclyl, aryl, heteroaryl, and aralkyl. More preferably, R₂ is independently selected from the group consisting of hydrogen, halo, C₁₋₄ alkyl, cycloalkyl, heterocyclyl, CN, OR₂₁, and N(R₂₁)₂; wherein R₂₁, independently from each other and at each occurrence, is selected from the group consisting of hydrogen, C₁₋₄ alkyl such as methyl, ethyl, propyl, isopropyl, butyl, tert-butyl, and isobutyl and C₃₋₆ cycloalkyl such as cyclopropyl, cyclobutyl, cyclopentyl and cyclohexyl. Even more preferably, R₂ is independently chosen from the group consisting of hydrogen, halo, C₁₋₄ alkyl, and N(R₂₁)₂ wherein R₂₁ is selected from the group consisting of hydrogen and C₁₋₄ alkyl such as methyl, ethyl, propyl, isopropyl, butyl, tert-butyl, and isobutyl.

In a preferred embodiment of the present invention, q in compound (C) of formulae (I) to (VII) is equal to 0 or 1.

According to certain embodiments of the present invention, x in compound (C) of formulae (II), (IV) or (VI) is an integer equal to 0 and y is an integer equal to 1.

According to certain embodiments of the present invention, x in compound (C) of formulae (II), (IV) or (VI) is an integer equal to 1 and y is an integer equal to 0.

According to certain embodiments of the present invention, x in compound (C) of formulae (II), (IV) or (VI) is an integer equal to 1 and y is an integer equal to 1.

According to certain embodiments of the present invention, x in compound (C) of formulae (II), (IV) or (VI) is an integer equal to 0 and y is an integer equal to 0.

In a preferred embodiment of the present invention, R₈ in compound (C) of formulae (I) is selected from the group consisting of C₆₋₁₂ alkyl, cycloalkyl and heterocyclyl, wherein said alkyl, cycloalkyl, and heterocyclyl are optionally substituted by a halogen atom, CF₃, N(R₁₁)₂, CN, or OR₁₁; and wherein each of R₁₁, independently from each other and at each occurrence, is selected from the group consisting of hydrogen, C₁₋₆ alkyl. More preferably, R₈ is C₆₋₁₂ alkyl, wherein said alkyl, is optionally substituted by a halogen atom. Even more preferably, R₈ is C₆₋₁₂ alkyl.

In a preferred embodiment of the present invention, Re in compound (C) of formulae (II) is selected from the group consisting of hydrogen, C₁₋₆ alkyl, cycloalkyl, N(R₁₁)₂, and CN, wherein said alkyl, and cycloalkyl, are optionally substituted by a halogen atom, CF₃, CN, or OR₁₁; and wherein each of R₁₁, independently from each other and at each occurrence, is selected from the group consisting of hydrogen, C₁₋₆ alkyl, C₂₋₆ alkenyl, and CF₃ wherein said alkyl, and alkenyl substituents are optionally substituted with an heteroaryl group optionally substituted with a C₁₋₄ alkyl with the proviso that if x=0 and y=0, R₉ is different from hydrogen and C₁₋₆ alkyl, wherein said alkyl is optionally substituted with heterocyclyl and N(R₁₁)₂; with the proviso that when x=0 and y=0, R₉ and R₂ may form together a saturated or an unsaturated cyclic moiety; with the proviso that when x=0 and y=0 and when R₉ and R₂ form together a saturated or an unsaturated cyclic moiety, R₉ is NR₁₁; with the proviso that when x=1 and y=1, R₉ is different from N(R₁₁)₂; and with the proviso that when x=0, y=0 and z=0, R₉ is different from pyrrole. More preferably, R₉ is selected from the group consisting of hydrogen, C₁₋₆ alkyl, cycloalkyl, N(R₁₁)₂, and CN, wherein said alkyl, and cycloalkyl, are optionally substituted by a halogen atom, CF₃, CN, or OR₁₁; and wherein each of R₁₁, independently from each other and at each occurrence, is selected from the group consisting of hydrogen, C₁₋₆ alkyl, C₂₋₆ alkenyl, and CF₃, wherein said alkyl, and alkenyl substituents are optionally substituted with an heteroaryl group optionally substituted with a C₁₋₄ alkyl; with the proviso that if x=0 and y=0, R₉ is different from hydrogen and C₁₋₆ alkyl, wherein said alkyl is optionally substituted with heterocyclyl and N(R₁₁)₂ and with the proviso that when x=0 and y=0, R₉ and R₂ may form together a saturated or an unsaturated cyclic moiety; with the proviso that when x=0 and y=0 and when R₉ and R₂ form together a saturated or an unsaturated cyclic moiety, R₉ is NR₁₁; with the proviso that when x=1 and y=1, R₉ is different from N(R₁₁)₂; and with the proviso that when x=0, y=0 and z=0, R₉ is different from pyrrole. Even more preferably, R₉ is selected from the group consisting of hydrogen, C₁₋₄ alkyl such as methyl, ethyl, propyl, isopropyl, butyl, tert-butyl, and isobutyl, a C₂₋₆ alkenyl such as propene or butene, C₃₋₆, cycloalkyl such as cyclopropyl, cyclobutyl, cyclopentyl and cyclohexyl, N(R₁₁)₂, and CN, wherein said alkyl, and cycloalkyl, are optionally substituted by a halogen atom, CF₃, CN, or OR₁₁; and wherein each of R₁₁, independently from each other and at each occurrence, is selected from the group consisting of hydrogen, C₁₋₆ alkyl, C₂₋₆ alkenyl, and CF₃, wherein said alkyl, and alkenyl substituents are optionally substituted with an heteroaryl group optionally substituted with a C₁₋₄ alkyl; with the proviso that if x=0 and y=0, R₉ is different from hydrogen and C₁₋₆ alkyl, wherein said alkyl is optionally substituted with heterocyclyl and N(R₁₁)₂ and with the proviso that when x=0 and y=0, R₉ and R₂ may form together a saturated or an unsaturated cyclic moiety; with the proviso that when x=0 and y=0 and when R₉ and R₂ form together a saturated or an unsaturated cyclic moiety, R₉ is NR₁₁; with the proviso that when x=1 and y=1, R₉ is different from N(R₁₁)₂; and with the proviso that when x=0, y=0 and z=0, R₉ is different from pyrrole.

In a preferred embodiment of the present invention, each of T in compound (C) of formulae (III) or (IV) is independently the moiety of formula (T-a) herein below:

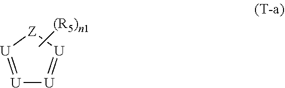

wherein:
each of U is preferably selected, independently from each other and at each occurrence, from C, C-halo, C—R, or N; wherein R is hydrogen or C₁₋₄ alkyl with the proviso that at least one U is different from N. More preferably, each of U is selected, independently from each other and at each occurrence, from C, C—R or N; wherein R is hydrogen or C₁₋₄ alkyl with the proviso that at least one U is different from N.
each of Z is, independently from each other and at each occurrence, preferably selected from the group consisting of CH₂, and O, S and NR₇ wherein R₇ is an hydrogen, or a C₁₋₄ alkyl such as methyl, ethyl, propyl, isopropyl, butyl, tert-butyl, and isobutyl. More preferably, each of Z is, independently from each other and at each occurrence, selected from the group consisting of CH₂, O, and NH.
each of R₅, independently from each other and at each occurrence is preferably selected from the group consisting of C₁₋₆ alkyl, cycloalkyl, heterocyclyl, aryl, heteroaryl, aralkyl, halo, CF₃, OR₁₁, N(R₁₁)₂, COOR₁₁, CO(R₁₁)₂, CON(R₁₁)₂, and each optional alkyl, cycloalkyl, aryl, heterocyclyl, heteroaryl, aralkyl substituent is further optionally substituted with halo, C₁₋₆ alkyl, cycloalkyl, aryl, heterocyclyl, N(R₁₁)₂, CN, OR₁₁, C(═O)OR₁₁, P(═O)(OR₁₁)₂, P(═O)(R₁₁)₂, CN or CF₃ and wherein each of R₁₁, independently from each other and at each occurrence, is selected from the group consisting of hydrogen, C₁₋₆ alkyl, cycloalkyl, and heterocyclyl. More preferably, each of R₅, independently from each other and at each occurrence, is selected from the group consisting of C₁₋₄ alkyl, cycloalkyl, heterocyclyl, CF₃, OR₁₁, and each optional alkyl, cycloalkyl, heterocyclyl, substituent is further optionally substituted with halo, C₁₋₄ alkyl, cycloalkyl, CN, OC₁₋₄ alkyl, C(═O)OC₁₋₄ alkyl, P(═O)(OC₁₋₄ alkyl)₂, P(═O)(C₁₋₄ alkyl)₂
n1 is preferably an integer equal to 0, 1 or 2. More preferably, n1 is an integer equal to 1 or 2.

In a preferred embodiment of the present invention, each of T in compound (C) of formulae (III) or (IV), independently from each other and at each occurrence is selected from the moiety of formula (T-a-1) to (T-a-11) herein below:

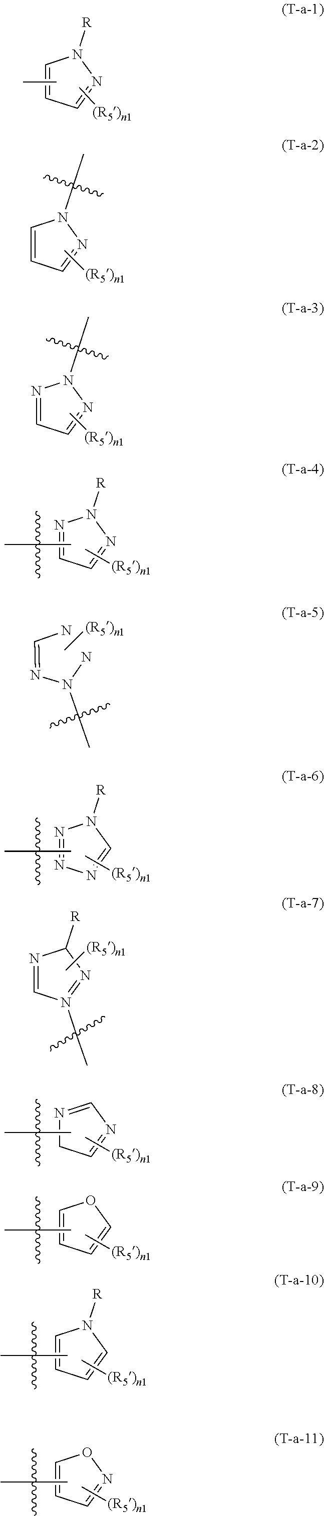

wherein each R is independently selected from the group consisting of hydrogen, C₁₋₄ alkyl, cycloalkyl, heterocyclyl, wherein said C₁₋₄ alkyl, cycloalkyl and heterocyclyl is optionally substituted with halo, CN, cycloalkyl, OC₁₋₄ alkyl, C(═O)OC₁₋₄ alkyl, P(═O)(C₁₋₄ alkyl)₂, P(═O)(OC₁₋₄ alkyl)₂ preferably R is hydrogen or methyl, and wherein each of R₅′ is independently selected from the group consisting of hydrogen, C₁₋₄ alkyl, CF₃, and cycloalkyl; and wherein n1 is an integer equal to 1 or 2.

In a preferred embodiment of the present invention, each of X in compound (C) of formulae (V) or (VI) is independently the moiety of formula (X-a) herein below:

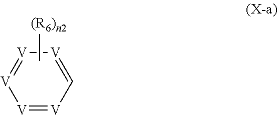

wherein:
each of V, independently from each other and at each occurrence, is selected from the group consisting of C, C-halo, C—R, and N; wherein R is hydrogen or C₁₋₄ alkyl; More preferably, each of V is selected, independently from each other and at each occurrence, from C, C—R or N; wherein R is hydrogen or C₁₋₄ alkyl.
each of R₆, independently from each other and at each occurrence is preferably selected from the group consisting of C₁₋₆ alkyl, cycloalkyl, heterocyclyl, aryl, heteroaryl, aralkyl, halo, CF₃, OR₁₁, N(R₁₁)₂, COOR₁₁, CO(R₁₁)₂, CON(R₁₁)₂, and each optional alkyl, cycloalkyl, aryl, heterocyclyl, heteroaryl, aralkyl substituent is further optionally substituted with halo, C₁₋₆ alkyl, cycloalkyl, aryl, heterocyclyl, N(R₁₁)₂, CN, OR₁₁, C(═O)OR₁₁, P(═O)(OR₁₁)₂, P(═O)(R₁₁)₂, CN or CF₃ and wherein each of R₁₁, independently from each other and at each occurrence, is selected from the group consisting of hydrogen, C₁₋₆ alkyl, cycloalkyl, and heterocyclyl. More preferably, each of R₆, independently from each other and at each occurrence, is selected from the group consisting of C₁₋₄ alkyl, cycloalkyl, heterocyclyl, heteroaryl, halo, CF₃, OR₁₁, N(R₁₁)₂ and each optional alkyl, cycloalkyl, heterocyclyl and heteroaryl substituent is further optionally substituted with halo, C₁₋₄ alkyl, cycloalkyl, heterocyclyl, CN, OC₁₋₄ alkyl, C(═O)OC₁₋₄ alkyl, P(═O)(OC₁₋₄ alkyl)₂, P(═O)(C₁₋₄ alkyl)₂, wherein said heterocyclyl is further optionally substituted with C₁₋₄ alkyl, and wherein each of R₁₁, independently from each other and at each occurrence, is selected from the group consisting of hydrogen, and C₁₋₄ alkyl. Even more preferably, each of R₆, independently from each other and at each occurrence, is selected from the group consisting of C₁₋₄ alkyl, cycloalkyl, heterocyclyl, heteroaryl, halo, CF₃, OR₁₁, N(R₁₁)₂ and each optional alkyl, cycloalkyl, heterocyclyl and heteroaryl substituent is further optionally substituted with C₁₋₄ alkyl, or heterocyclyl, wherein said heterocyclyl is further optionally substituted with C₁₋₄ alkyl, and wherein each of R₁₁, independently from each other and at each occurrence, is selected from the group consisting of hydrogen, and C₁₋₄ alkyl.
n2 is preferably an integer equal to 0, 1 or 2. More preferably, n1 is an integer equal to 0 or 1.

In one embodiment of the present invention, each of X in compound (C) of formulae (V) or (VI) is independently selected from the moiety of formula (X-a-1) to (X-a-3) herein below:

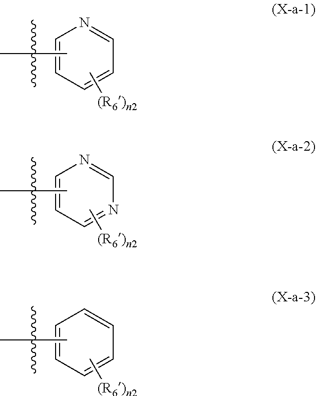

wherein
each of R₆′ is independently selected from hydrogen, halo, C₁₋₄alkyl, OC₁₋₄alkyl, NH₂, N(C₁₋₄alkyl)₂, heterocyclyl, heteroaryl, wherein said C₁₋₄ alkyl, heteroaryl and heterocyclyl are optionally substituted with halo, C₁₋₄alkyl, heterocyclyl which is optionally substituted with C₁₋₄ alkyl
n2 is an integer equal to 1 or 2.

In a preferred embodiment of the present invention, the dash bond in compound (C) of formulae (VII) represents a triple bond.

In a preferred embodiment of the present invention, Rₐ₁ in compound (C) of formulae (VII) is independently selected from the group consisting of C₁₋₄ alkyl, cycloalkyl, heterocyclyl, aryl, heteroaryl, aralkyl, wherein said alkyl cycloalkyl, heterocyclyl, aryl, heteroaryl and aralkyl are optionally substituted by halo, NO₂, C₁₋₄ alkyl, cycloalkyl, heterocyclyl, phenyl, heteroaryl, CF₃, CN, OR₁₁, N(R₁₁)₂, and wherein each of R₁₁ is selected from the group consisting of hydrogen, or C₁₋₄ alkyl. More preferably, Rₐ₁ is independently selected from the group consisting of C₁₋₄ alkyl, cycloalkyl, heterocyclyl, aryl, heteroaryl, aralkyl, wherein said alkyl cycloalkyl, heterocyclyl, aryl, heteroaryl and aralkyl are optionally substituted by halo, heterocyclyl, aryl, heteroaryl, OR₁₁, N(R₁₁)₂, and wherein each of R₁₁ is selected from the group consisting of hydrogen and C₁₋₄ alkyl. Even more preferably, Rₐ₁ is independently C₁₋₄ alkyl, wherein said alkyl, is optionally substituted by aryl, heteroaryl, OR₁₁, N(R₁₁)₂, and wherein each of R₁₁ is selected from the group consisting of hydrogen, and C₁₋₄ alkyl.

In a preferred embodiment of the present invention, Rₐ₂ in compound (C) of formulae (VII) is independently selected from the group consisting of C₁₋₄ alkyl, cycloalkyl, heterocyclyl, aryl, heteroaryl, aralkyl, wherein said alkyl cycloalkyl, heterocyclyl, aryl, heteroaryl and aralkyl are optionally substituted by halo, NO₂, C₁₋₄ alkyl, cycloalkyl, heterocyclyl, phenyl, heteroaryl, CF₃, CN, OR₁₁, N(R₁₁)₂, and wherein each of R₁₁ is selected from the group consisting of hydrogen, or C₁₋₄ alkyl. More preferably, Rₐ₂ is independently selected from the group consisting of C₁₋₄ alkyl, cycloalkyl, heterocyclyl, aryl, heteroaryl, aralkyl, wherein said alkyl cycloalkyl, heterocyclyl, aryl, heteroaryl and aralkyl are optionally substituted by halo, heterocyclyl, phenyl, heteroaryl, OR₁₁, N(R₁₁)₂, and wherein each of R₁₁ is selected from the group consisting of hydrogen, or C₁₋₄ alkyl. Even more preferably, Rₐ₂ is independently C₁₋₄ alkyl, wherein said alkyl, is optionally substituted by aryl, heteroaryl, OR₁₁, N(R₁₁)₂, and wherein each of R₁₁ is selected from the group consisting of hydrogen, and C₁₋₄ alkyl.

In a preferred embodiment of the present invention, n3 in compound (C) of formulae (VII) is an integer equal to 0.

According to one embodiment of the present invention, the compound (C) according to formula (II), or the N-oxide, pharmaceutically acceptable salt, pharmaceutically acceptable solvate, or stereoisomer thereof, preferably is a compound chosen among those of formulae (II-a) or (II-b) [compound (C) of class (II) herein after]:

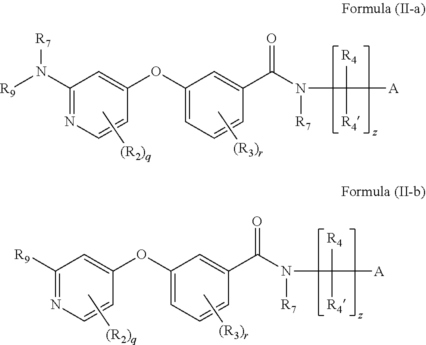

wherein A, R₄, R₄′, z, R₇, R₃, r, R₂, q and R₉ have the same meaning as defined above for formula (II).

According to one embodiment of the present invention, the compound (C) according to formula (III), or the N-oxide, pharmaceutically acceptable salt, pharmaceutically acceptable solvate, or stereoisomer thereof, preferably is a compound of formulae (III-a) [compound (C) of class (III) herein after]:

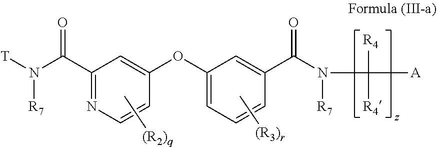

wherein A, R₄, R₄′, z, R₇, R₃, r, R₂, q, and T have the same meaning as defined above for formula (III).

According to one embodiment of the present invention, the compound (C) according to formula (IV), or the N-oxide, pharmaceutically acceptable salt, pharmaceutically acceptable solvate, or stereoisomer thereof, preferably is a compound chosen among those of formulae (IV-a) to (IV-c) [compound (C) of class (IV) herein after]:

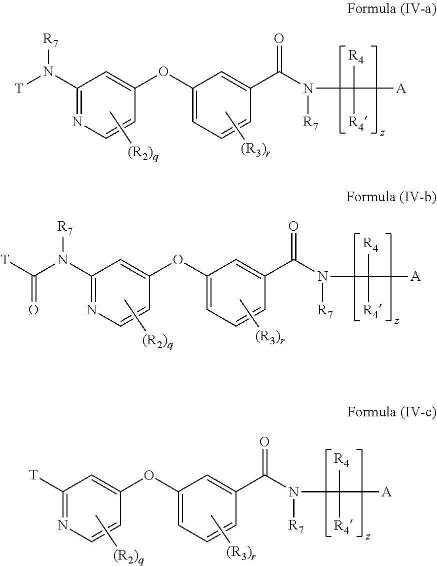

wherein A, R₄, R₄′, z, R₇, R₃, r, R₂, q, and T have the same meaning as defined above for formula (IV).

According to one embodiment of the present invention, the compound (C) according to formula (VI), or the N-oxide, pharmaceutically acceptable salt, pharmaceutically acceptable solvate, or stereoisomer thereof, preferably is a compound chosen among those of formulae (VI-a) to (VI-c) [compound (C) of class (VI) herein after]:

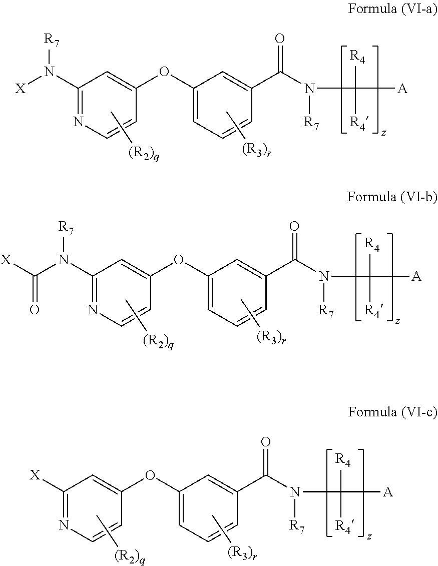

wherein A, R₄, R₄′, z, R₇, R₃, r, R₂, q, and X have the same meaning as defined above for formula (VI).

According to one embodiment of the present invention, the compound (C) according to formula (VII), or the N-oxide, pharmaceutically acceptable salt, pharmaceutically acceptable solvate, or stereoisomer thereof, preferably is a compound chosen among those of formulae (VII-a) or (VII-b) [compound (C) of class (VI) herein after]:

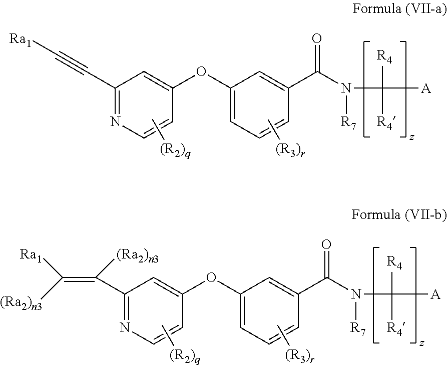

wherein A, R₄, R₄′, z, R₇, R₃, r, R₂, q, Rₐ₁, Rₐ₂ and n3 have the same meaning as defined above for formula (VII).

In compounds (C) according to the present invention, preferably R₄′ and R₇ are hydrogen and r and q are equal to 1. Preferred compounds (C) of class (II) are thus selected from those of formulae (II-a-1) to (II-c-1) herein below:

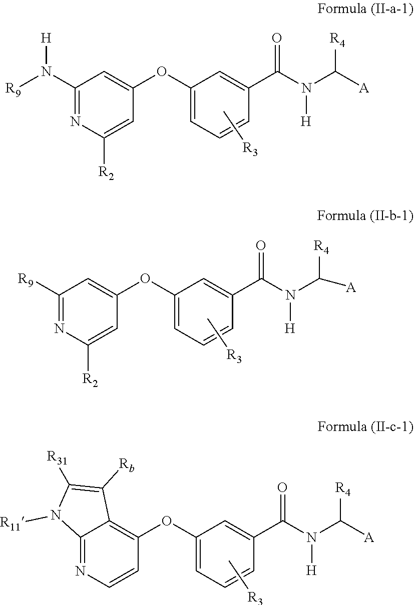

wherein A, R₄, R₃, R₂, and R₉ have the same meaning as defined above for formula (II); wherein R₃₁ is a heteroaryl which is optionally substituted with a C₁₋₄ alkyl, wherein R₁₁′ is hydrogen or C₁₋₄ alkyl; and wherein R_{b} is selected from the group consisting of hydrogen, halo, C₁₋₄ alkyl, and C₁₋₆ cycloalkyl, wherein said heteroaryl is a monocyclic ring structure containing 5 or 6 ring atoms, or a bicyclic aromatic group having 8 to 10 atoms, containing 1-3 heteroatoms independently selected from O or N.

In one embodiment of the present invention, the compounds (C) of class (II) are selected from those of formulae (II-a-1) to (II-c-1).

In compounds (C) according to the present invention, preferably R₄′ and R₇ are hydrogen and r and q are equal to 1. Preferred compounds (C) of class (IV) are thus selected from those of formula (IV-a-1) to (IV-c-1) herein below:

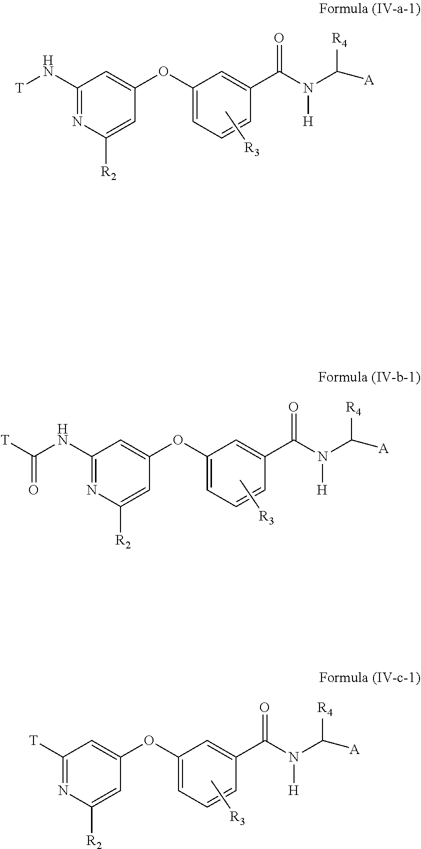

wherein A, R₄, R₃, R₂, and T have the same meaning as defined above for formula (IV).

In one embodiment of the present invention, the compounds (C) of class (IV) are selected from those of formula (IV-a-1) to (IV-c-1).

In compounds (C) according to the present invention, preferably R₄′ and R₇ are hydrogen and r and q are equal to 1. Preferred compounds (C) of class VI are thus selected from those of formula (VI-a-1) to (VI-c-1) herein below:

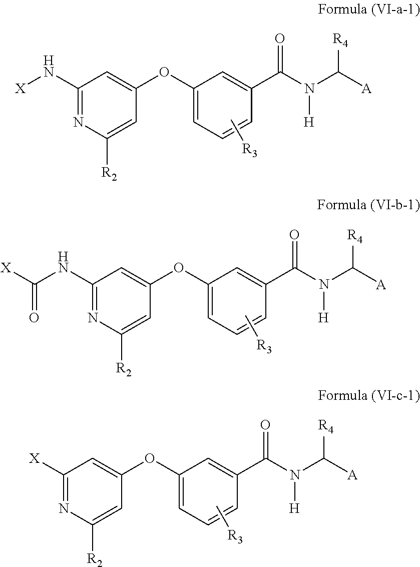

wherein A, R₄, R₃, R₂, and X have the same meaning as defined above for formula (VI).

In one embodiment of the present invention, the compounds (C) of class (VI) are selected from those of formula (VI-a-1) to (VI-c-1).

In compounds (C) according to the present invention, preferably R₄′ and R₇ are hydrogen and r and q are equal to 1. Preferred compounds (C) of class (VII) are thus selected from those of formulae (VII-a-1) or (VII-b-1) herein below:

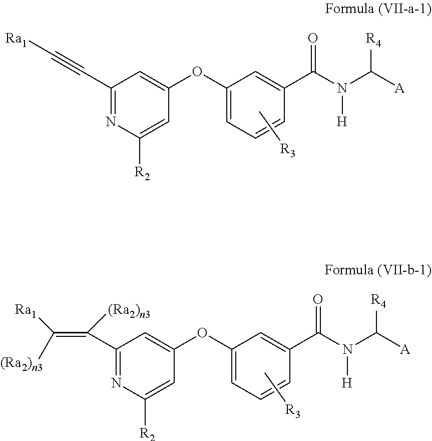

wherein A, R₄, R₃, R₂, Rₐ₁, Rₐ₂, and n3 have the same meaning as defined above for formula (VII).

In one embodiment of the present invention, the compounds (C) of class (II) are selected from those of formulae (VII-a-1) or (VII-b-1).

In a preferred embodiment of the present invention, the compound (C) of class (II) according to the present invention are selected from those of formula (II-a-2) or (II-b-2) or (II-c-2) herein below:

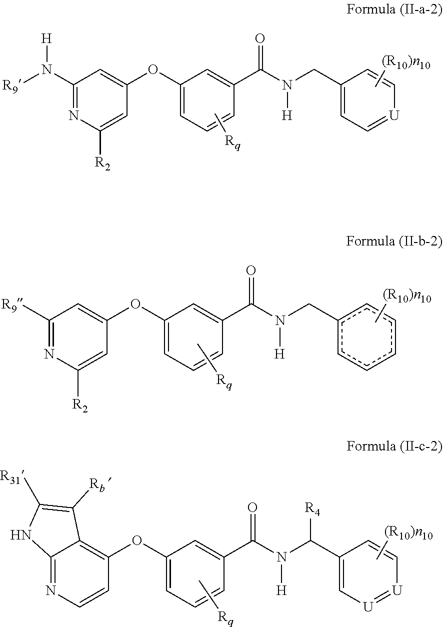

wherein:
each of R₉′ is selected from the group consisting of hydrogen, CN and C₃₋₆ cycloalkyl such as cyclopropyl;
each of R₉″ is selected from the group consisting of hydrogen, C₁₋₄ alkyl, CN and C₃₋₆ cycloalkyl such as cyclopropyl;
each of R₂ is independently selected from hydrogen or halo;
each of R₉ is independently selected from the group consisting of hydrogen, CH₃, OCH₃, and halo, such as F or Cl.
each of R₁₀ is independently selected from the group consisting of H, F, Cl, OCH₃, or CF₃;
each of U is selected from the group consisting of C, C—R₁₀ and N;
n10 is an integer equal to 0, 1 or 2; and
each of R₃₁′ is selected from the group consisting of pyrazyl, N-methylpyrazyl, and pyridyl.
R_{b}′ is selected from the group consisting of hydrogen, halo, C₁₋₄ alkyl, and C₁₋₄ cycloalkyl; preferably R_{b}′ is selected from the group consisting of CI, CH₃, and cyclopropyl.
the dash bond represents an optional double bond.

In a preferred embodiment of the present invention, the compound (C) of class (IV) according to the present invention are selected from those of formula (IV-a-2-1), (IV-a-2-2), (IV-b-2-1), (IV-b-2-2), or (IV-c-2) to (IV-c-2-4) herein below:

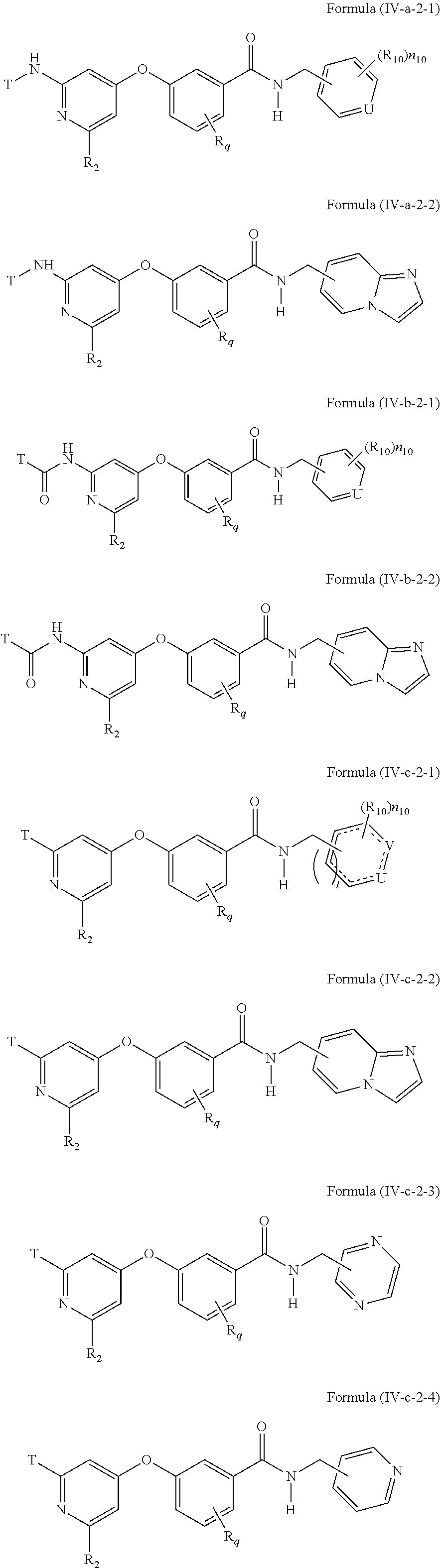

wherein:
T is, independently from each other and at each occurrence, selected from the moiety of formula (T-a-a) to (T-a-f) herein below:

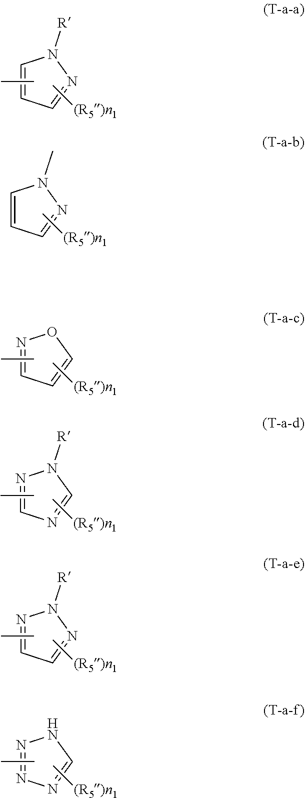

wherein:
each of R′ is independently hydrogen, C₁₋₄ alkyl, cycloalkyl selected from the group consisting of cyclopropyl and cyclobutyl; heterocyclyl selected from the group consisting of oxetanyl, tetrahydropyranyl, azetdinyl, and piperidinyl; wherein said alkyl is further optionally substituted with F, OC₁₋₄ alkyl, P(═O)(OC₁₋₄alkyl)₂, P(═O)(C₁₋₄alkyl)₂, CN, cyclopropyl, or cyclobutyl; and wherein said heterocyclyl is further optionally substituted with C(═O)(OC₁₋₄alkyl),
each of R″5 is independently selected from the group consisting of hydrogen, C₁₋₄ alkyl, CF₃ and cyclopropyl;
each of n1, independently from each other and at each occurrence is an integer equal to 0, 1 or 2.
R₂ is independently hydrogen, halo, or NH₂;
each of R₉ is independently selected from the group consisting of H, CH₃, OCH₃, and halo, such as F or Cl;
each of R₁₀ is independently selected from the group consisting of hydrogen, halo, C₁₋₄ alkyl, CF₃, OC₁₋₄alkyl, and CN,
each of U and V are independently C, C—R₁₀ or N;
n10 is an integer equal to 0, 1 or 2.

In a preferred embodiment of the present invention, the compound (C) of class (VI) according to the present invention are selected from those of formula (VI-a-2) to (VI-c-2) herein below:

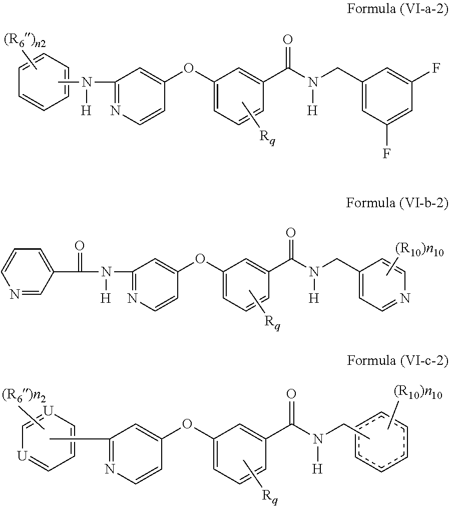

wherein
each of R″₆ is independently selected from the group consisting of hydrogen, halo, C₁₋₄ alkyl, N(R₂₁)₂, OR₂₁; heterocyclyl selected from the group consisting of pyrrolidyl, piperidyl, morpholinyl, piperazyl; a pyrazyl wherein said heterocyclyl and pyrazyl are optionally substituted with C₁₋₄ alkyl, and wherein R₂₁ is a C₁₋₄ alkyl.
each of R_{q} is independently selected from the group consisting of H, CH₃, OCH₃, and halo, such as F or Cl;
each of R₁₀ is independently selected from the group consisting of hydrogen, halo, OC₋₄ alkyl, and CN;
each of U is independently C, C—R₁₀ or N;
n₁₀ is an integer equal to 0, 1 or 2
n₂ is an integer equal to 0, 1 or 2.

In a preferred embodiment of the present invention, the compound (C) of class (VII) according to the present invention are selected from those of formula (VII-a-2) herein below:

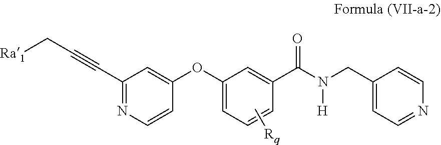

wherein Ra′₁ is selected from the group consisting of benzyl, pyrazyl, OH, OC₁₋₄ alkyl, NH₂, and NH(C₁₋₄ alkyl) and wherein R_{q} is selected from the group consisting of H, CH₃, OCH₃, and halo, such as F or Cl; preferably R_{q} is H or CH₃.

In a preferred embodiment of the present invention, the compound (C) according to general formula (II-a) is a compound chosen among those of formulae (VIII) to (XXXII-3) herein below:

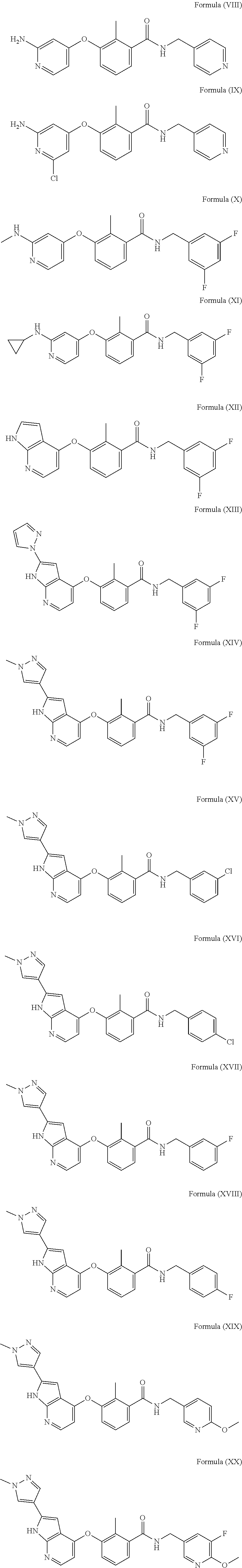

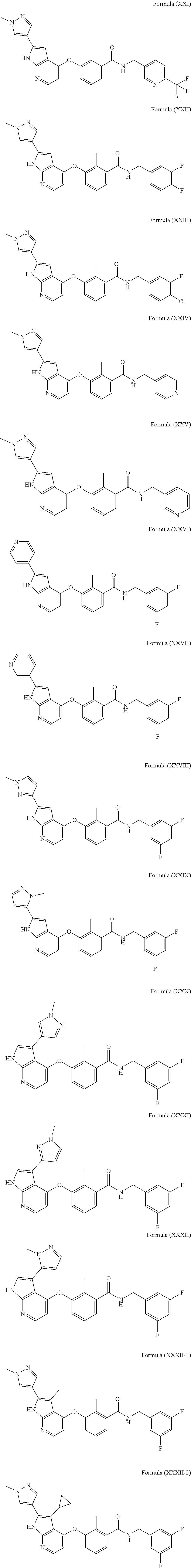

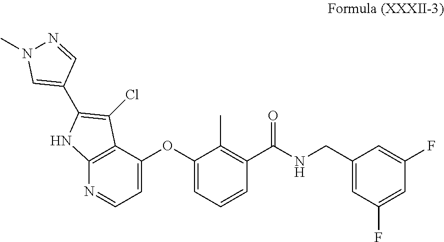

In a preferred embodiment of the present invention, the compound (C) according to general formula (II-b) is a compound chosen among those of formulae (XXXIII) to (XXXIV) herein below:

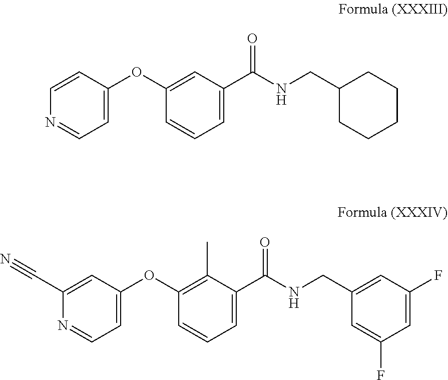

In a preferred embodiment of the present invention, the compound (C) according to general formula (III-a) is a compound chosen among those of formulae (XXXV) to (XXXVI) herein below:

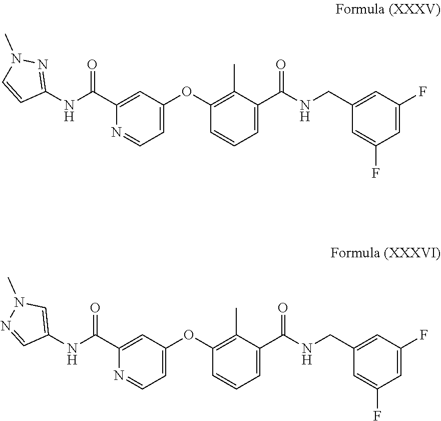

In a preferred embodiment of the present invention, the compound (C) according to general formula (IV-a) is a compound chosen among those of formulae (XXXVII) to (LXXI-2) herein below:

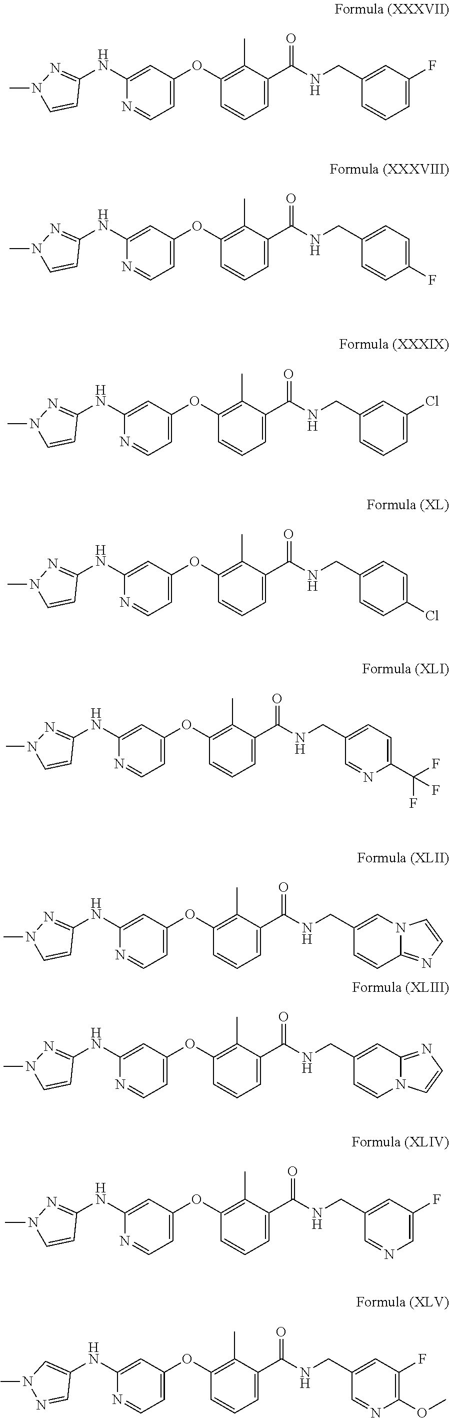

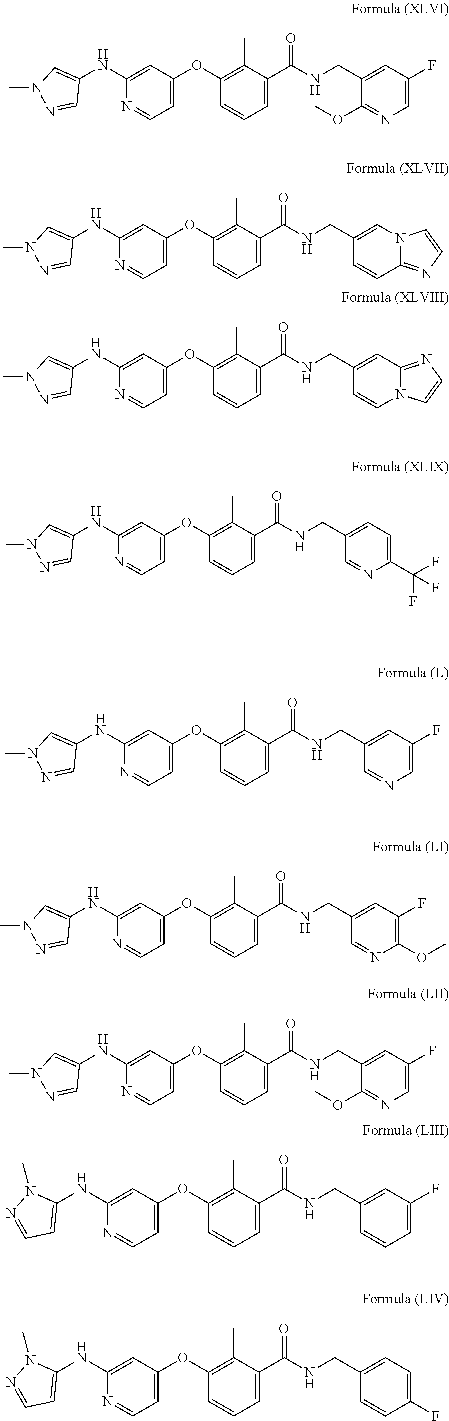

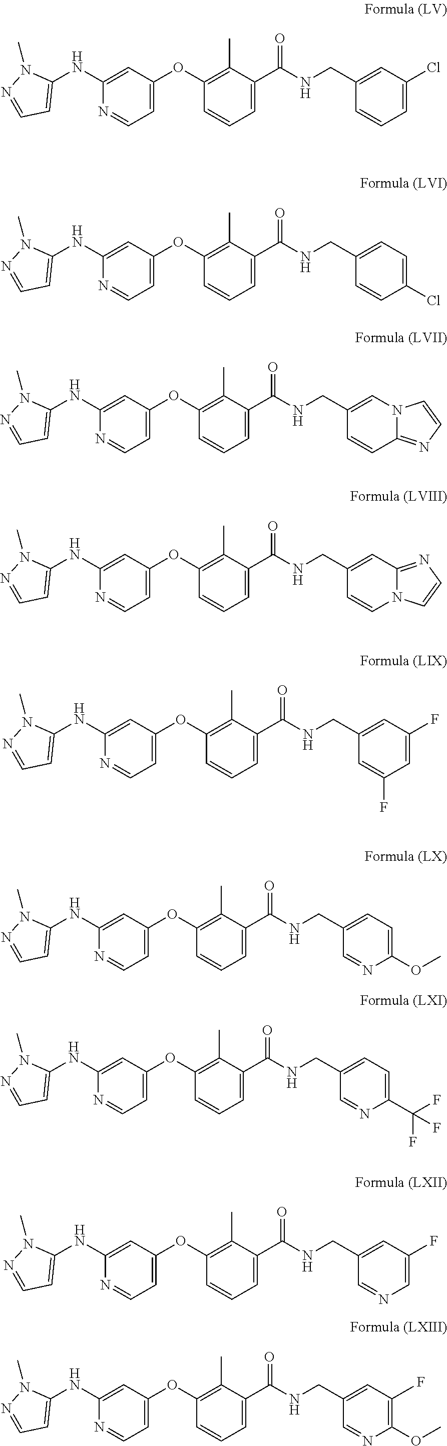

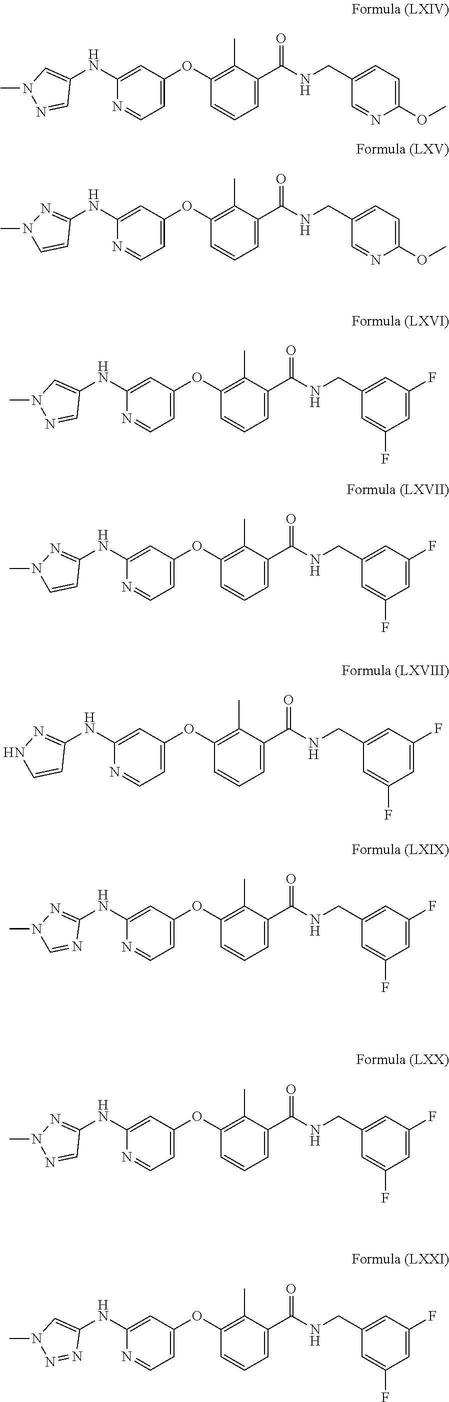

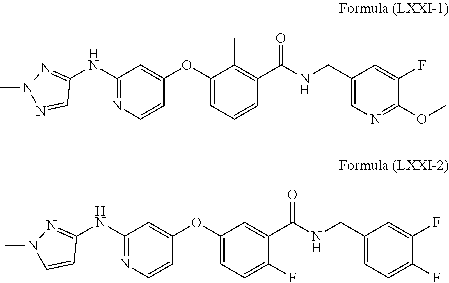

In a preferred embodiment of the present invention, the compound (C) according to general formula (IV-b) is a compound chosen among those of formulae (LXXII) to (CV) herein below:

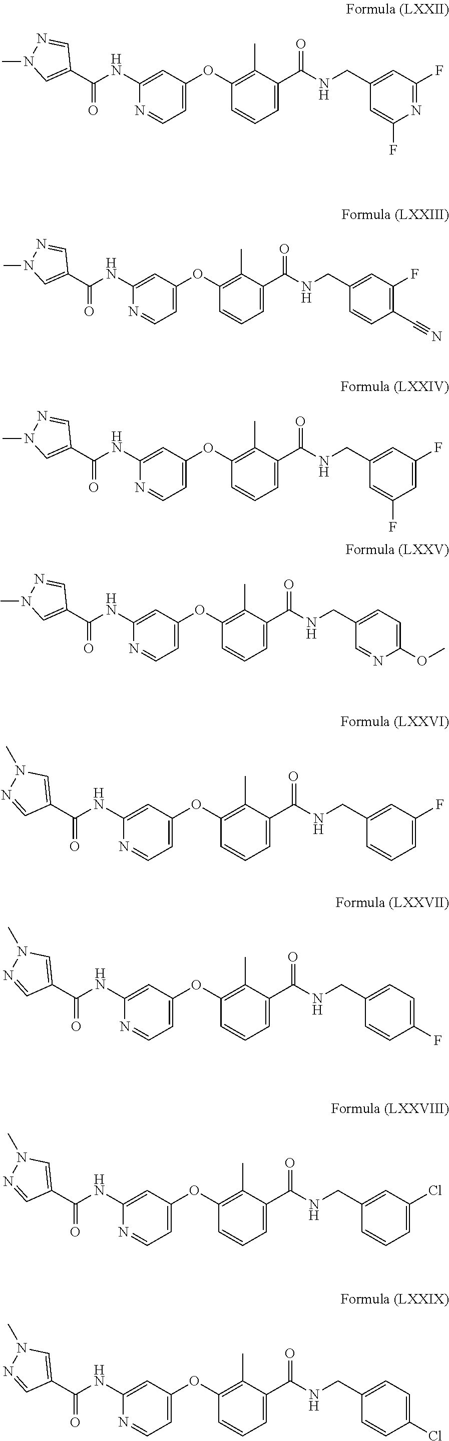

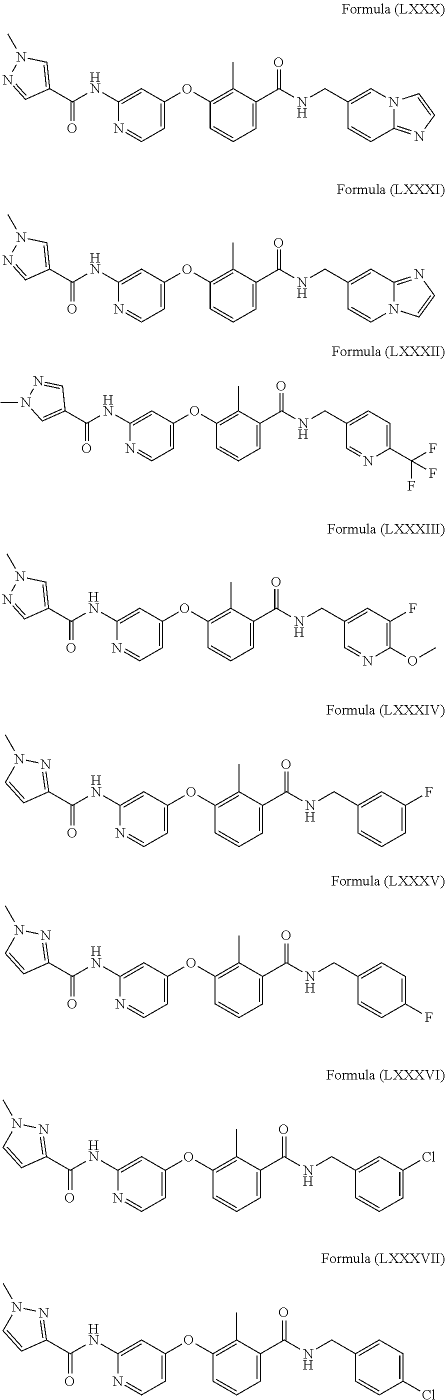

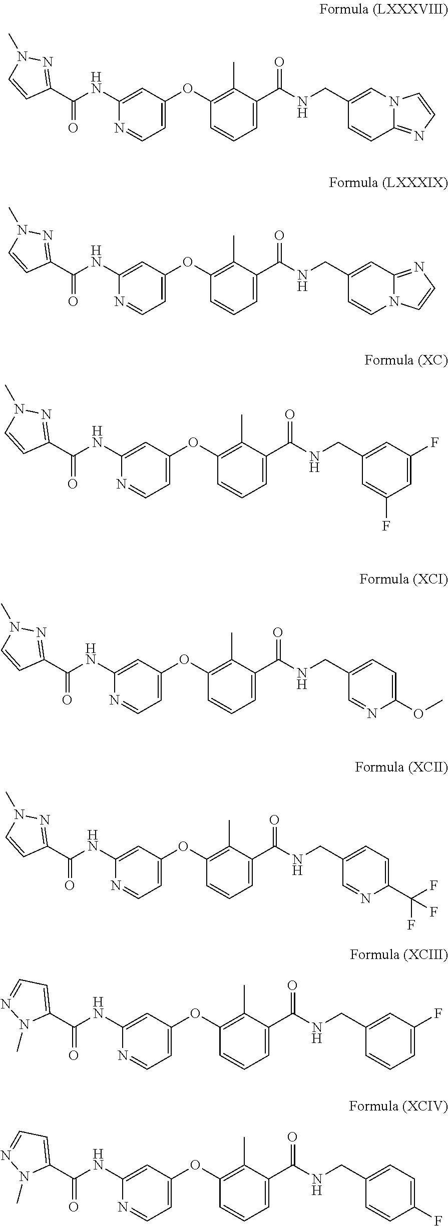

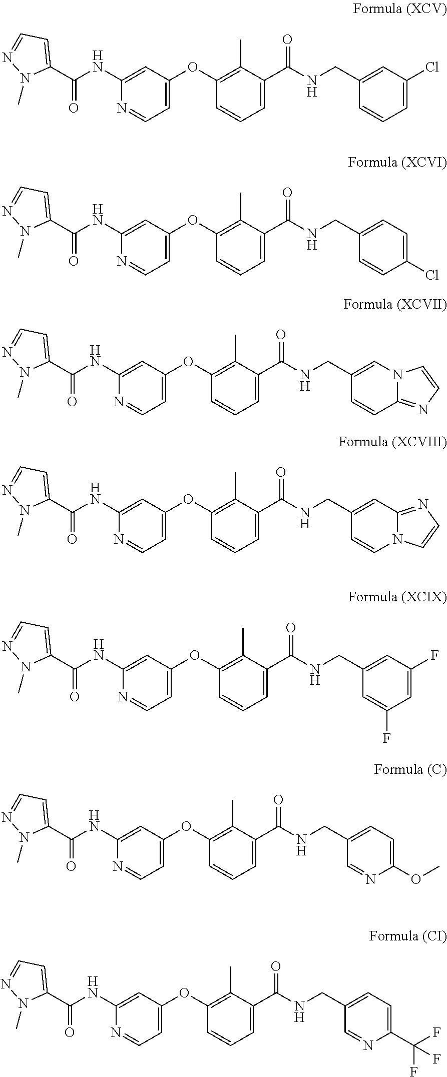

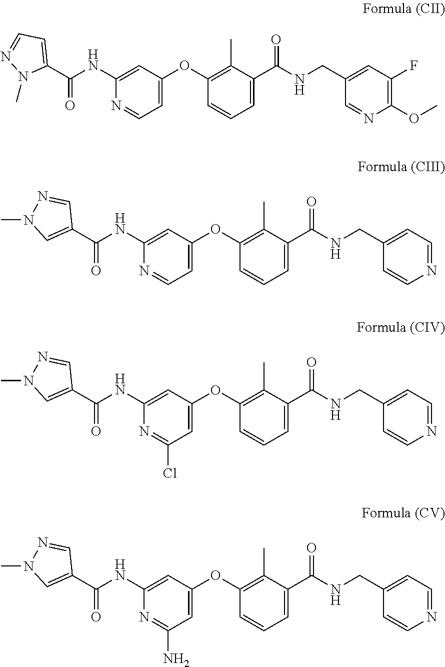

In a preferred embodiment of the present invention, the compound (C) according to general formula (IV-c) is a compound chosen among those of formulae (CVI) to (CXCVIII-5) herein below:

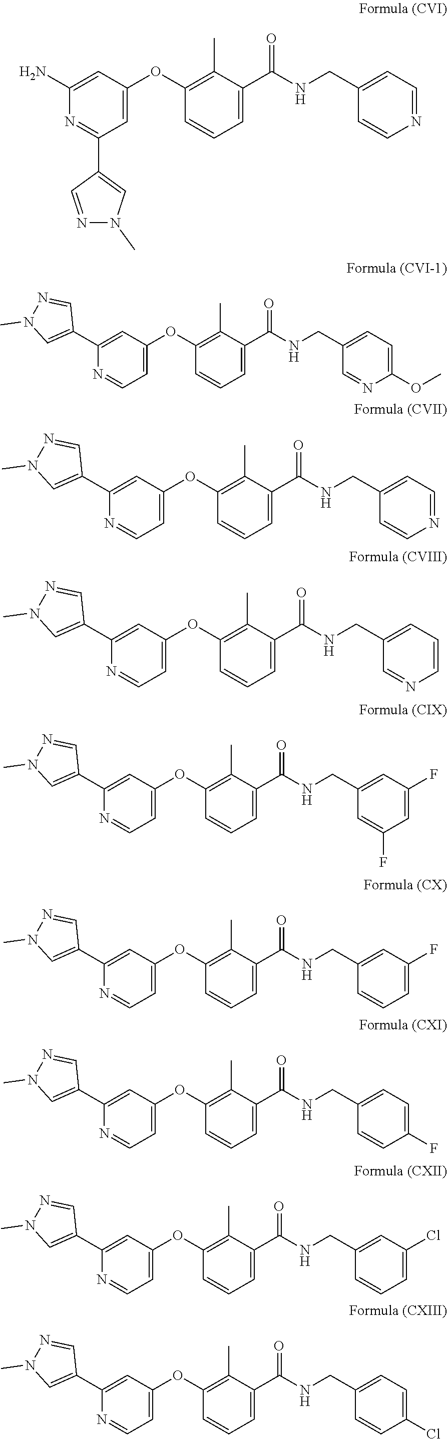

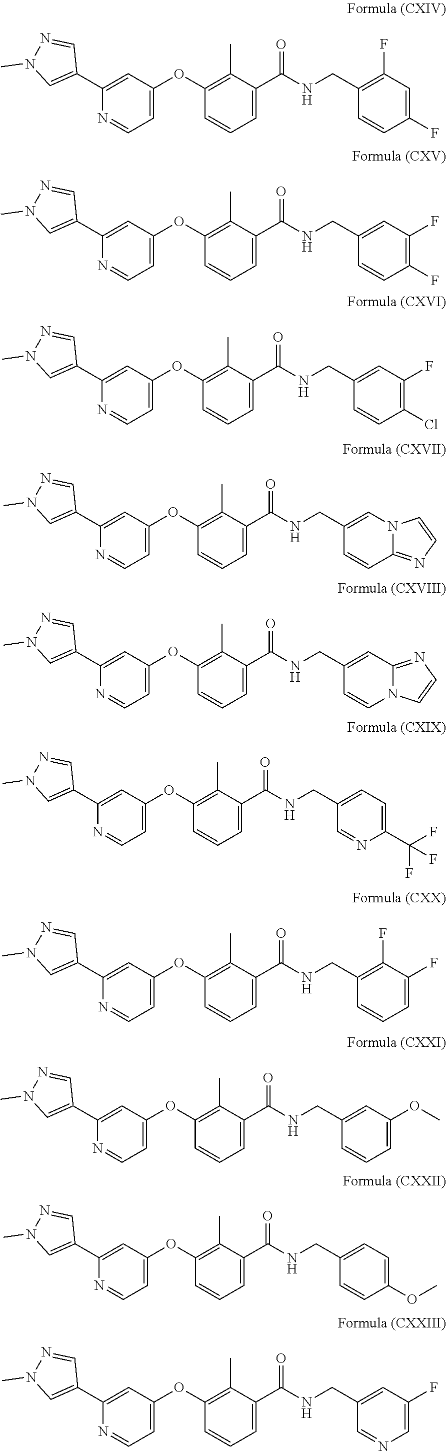

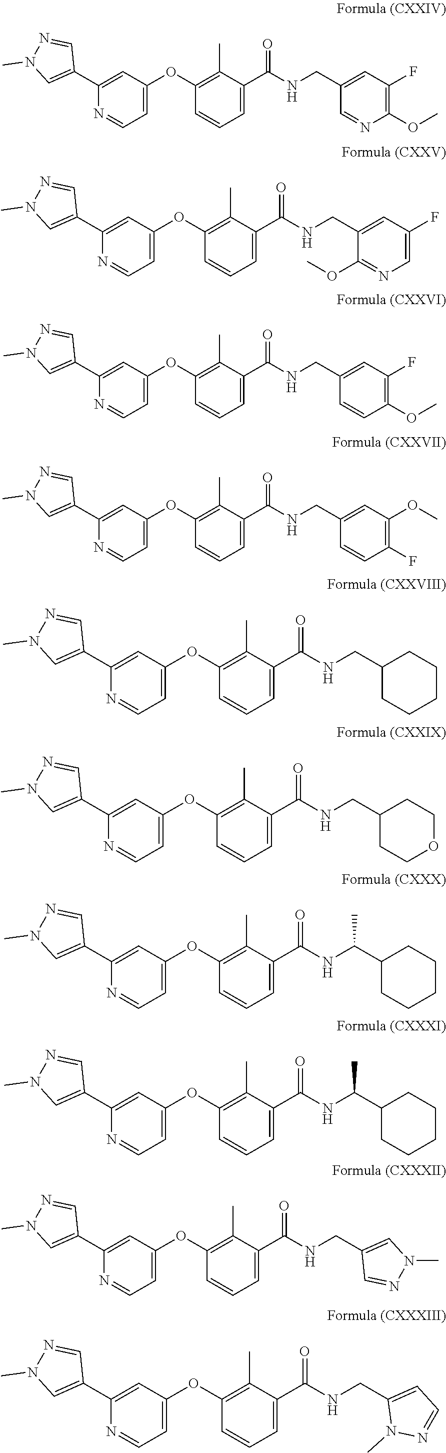

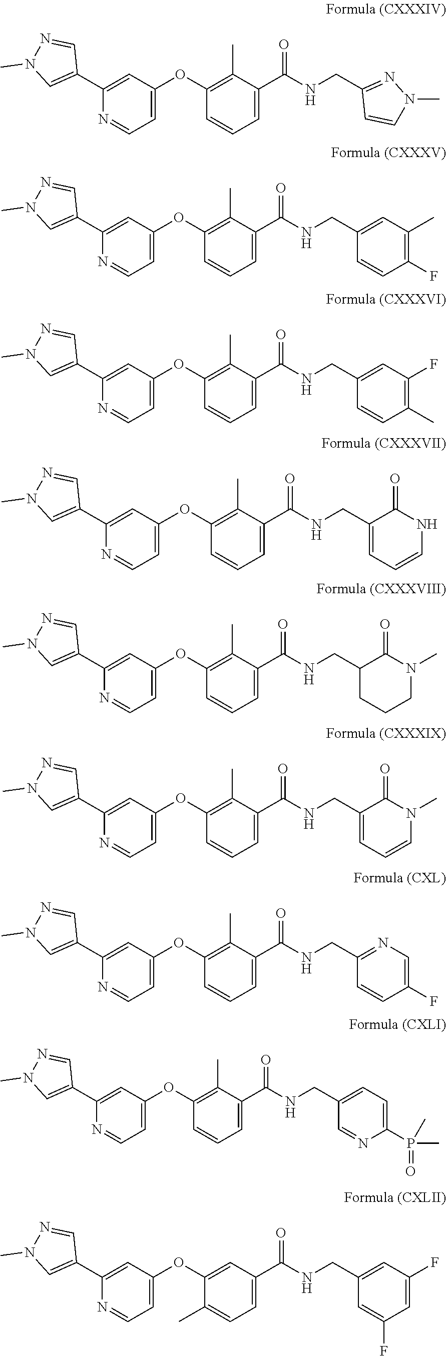

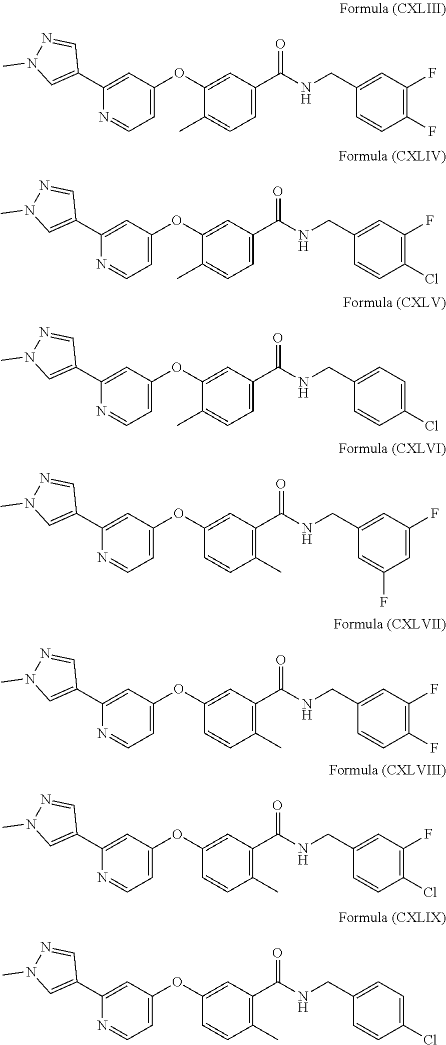

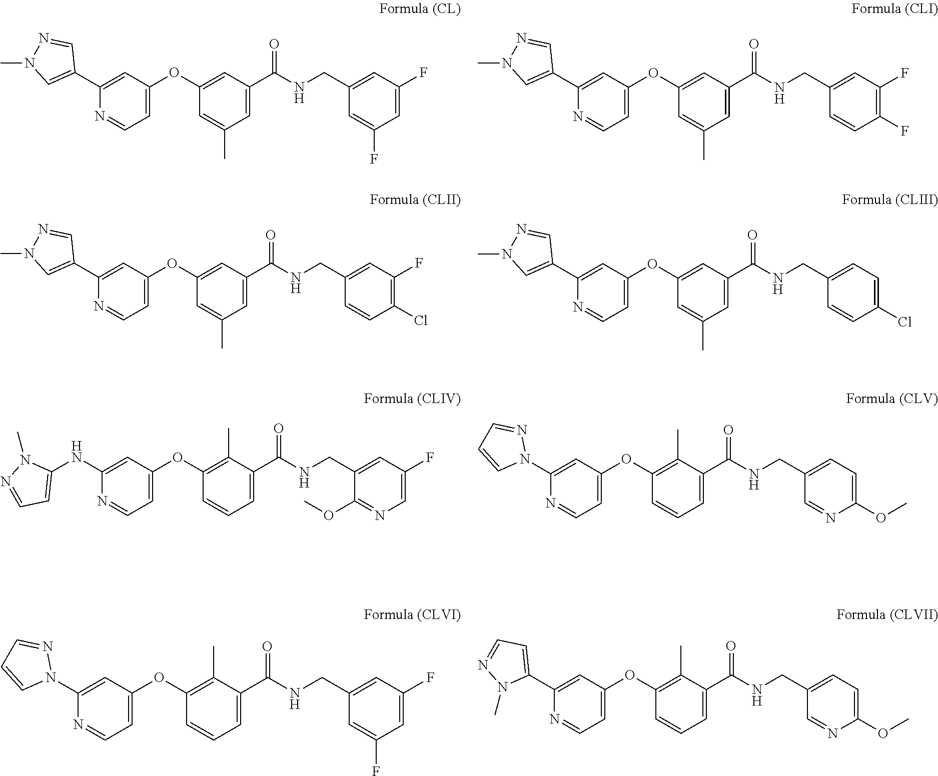

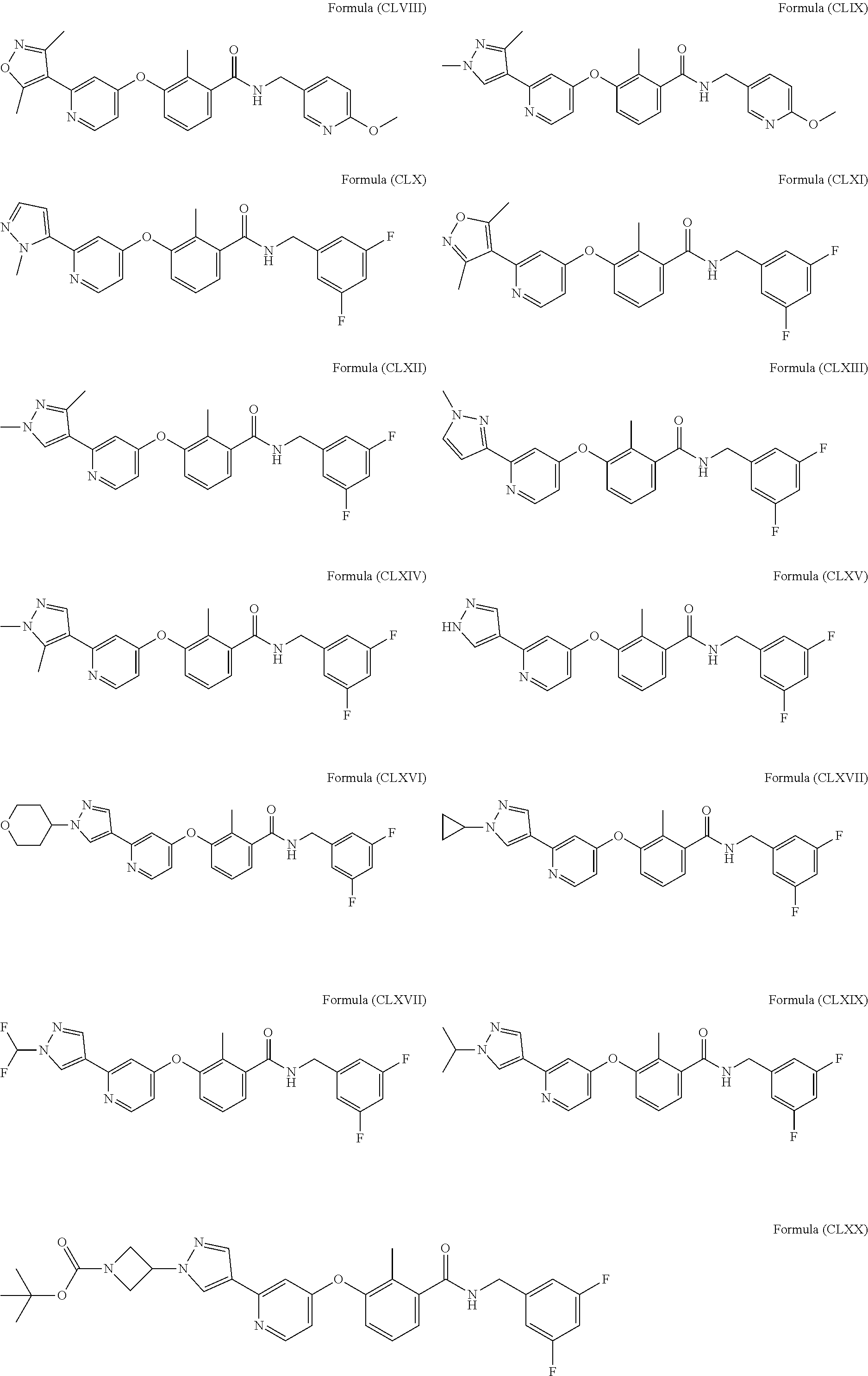

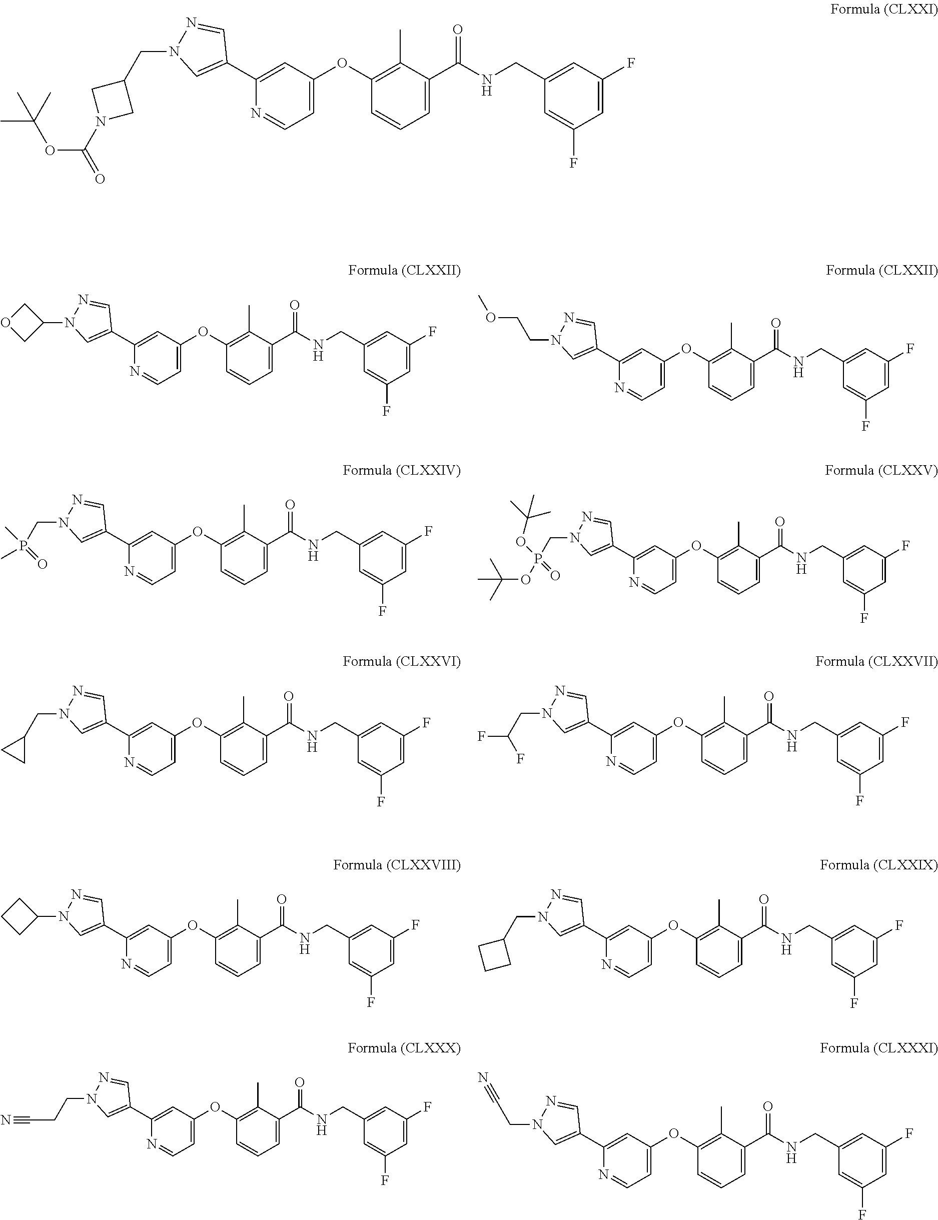

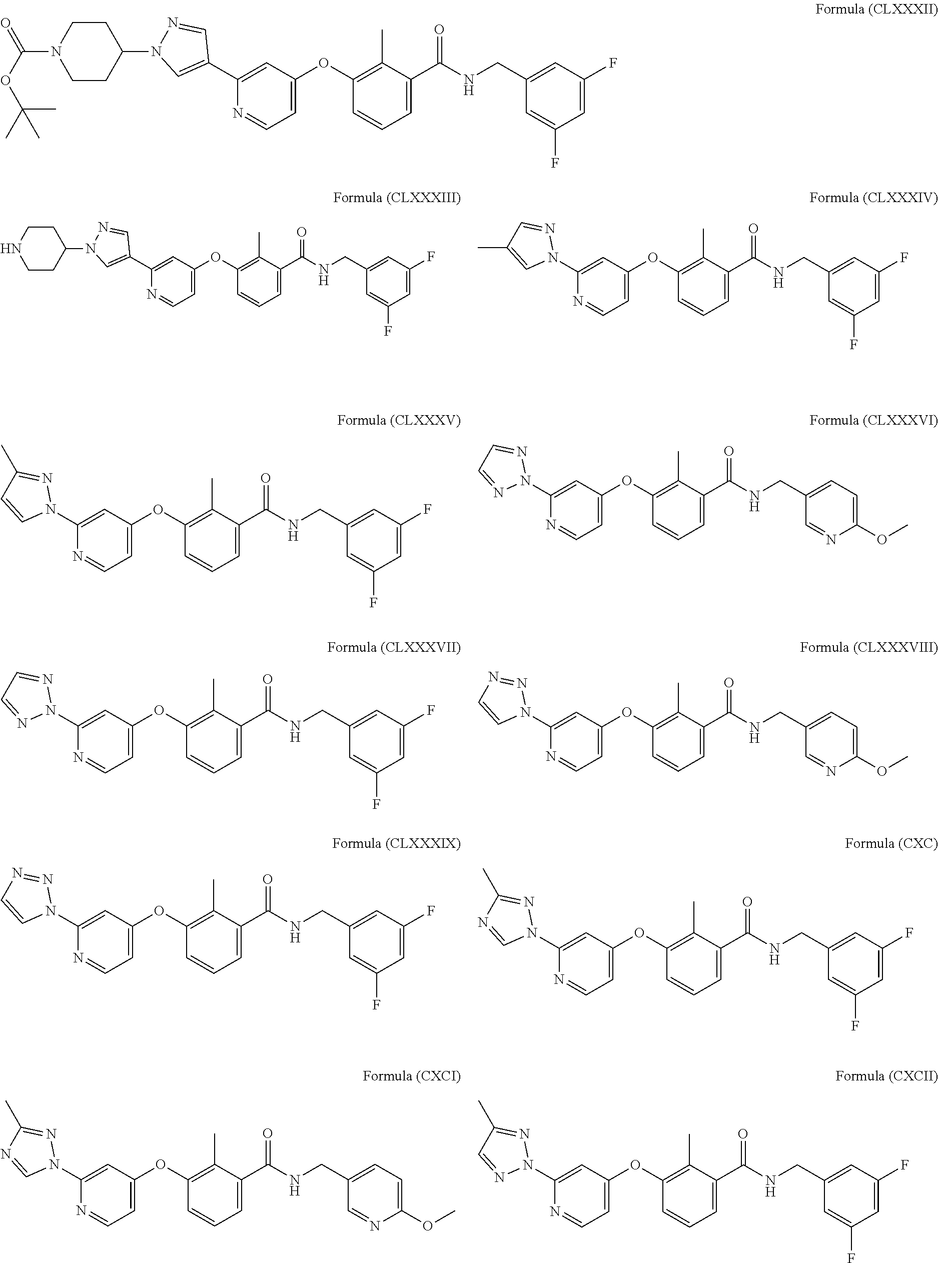

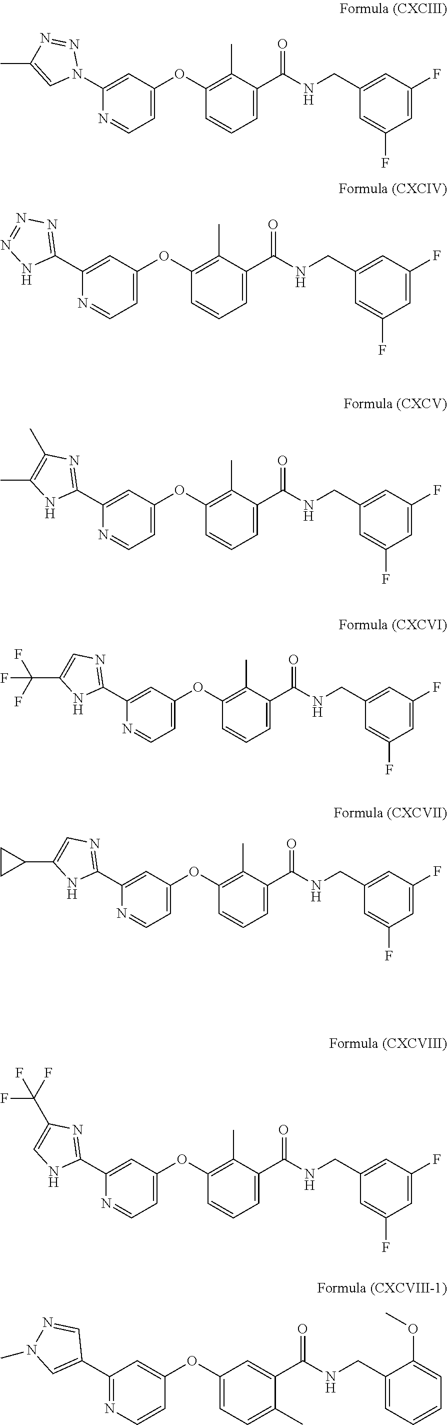

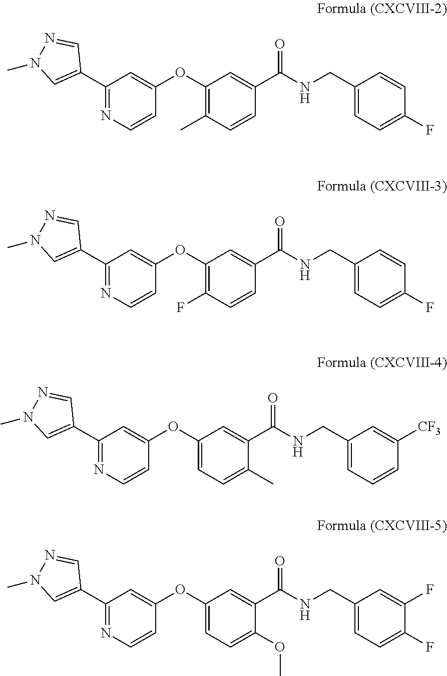

In a preferred embodiment of the present invention, the compound (C) according to general formula (VI-a) is a compound chosen among those of formulae (CXCIX) to (CCXII) herein below:

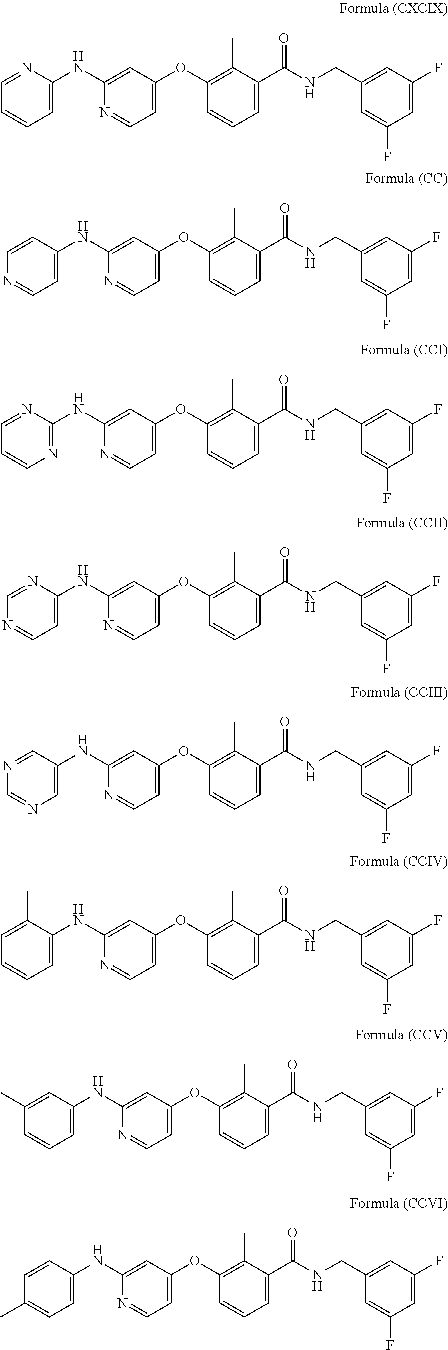

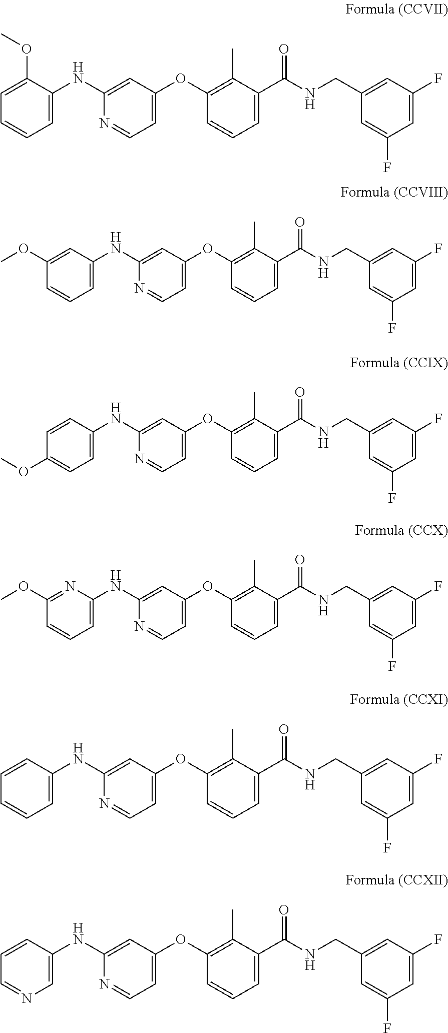

In a preferred embodiment of the present invention, the compound (C) according to general formula (VI-b) is a compound chosen among those of formulae (CCXIII) to (CCXV) herein below:

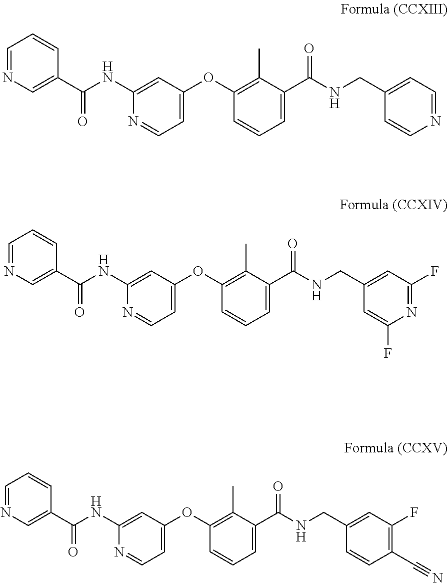

In a preferred embodiment of the present invention, the compound (C) according to general formula (VI-c) is a compound chosen among those of formulae (CCXVI) to (CCLX) herein below:

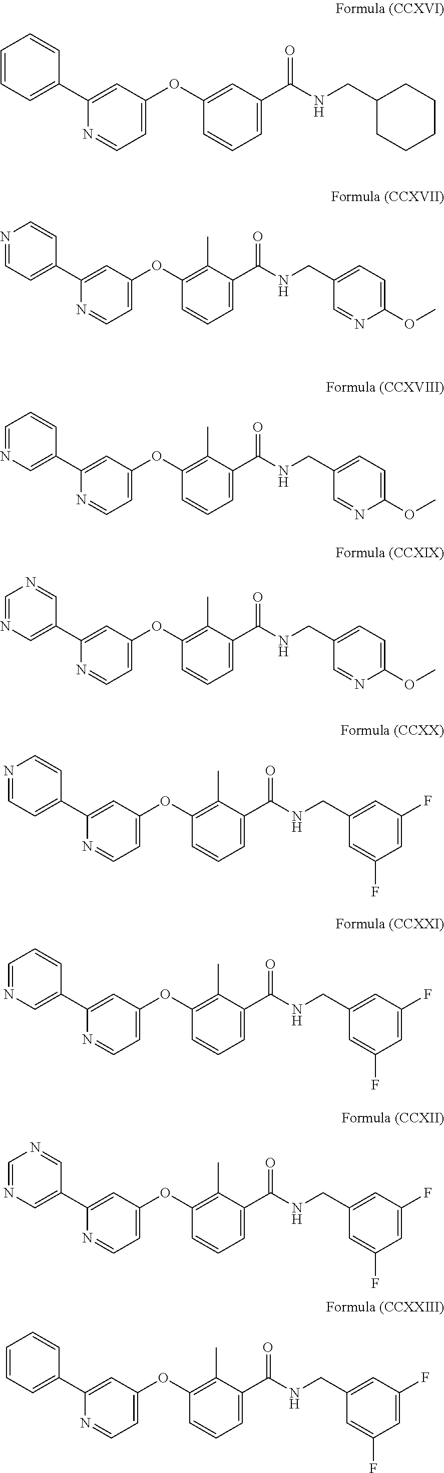

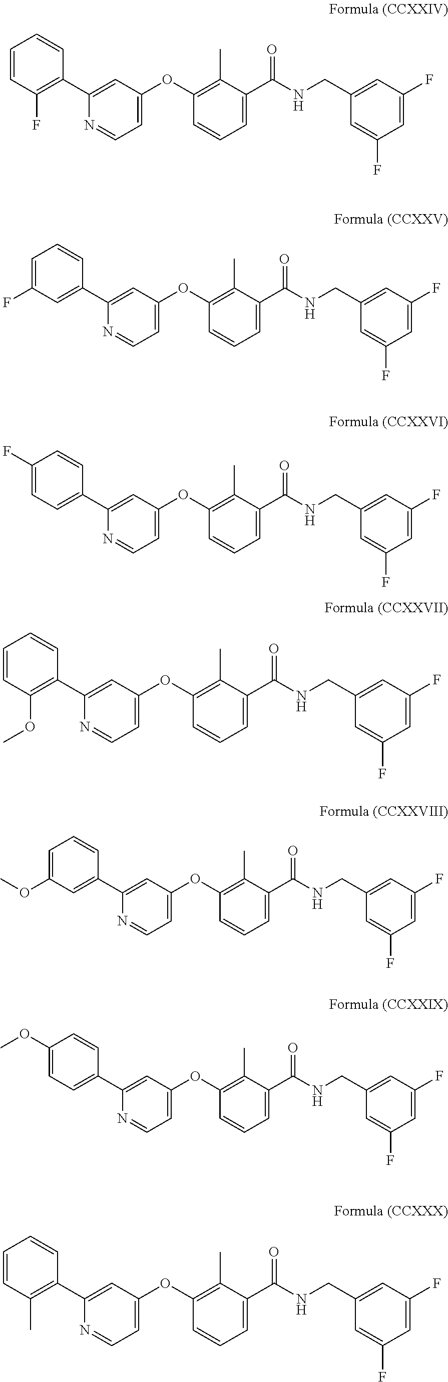

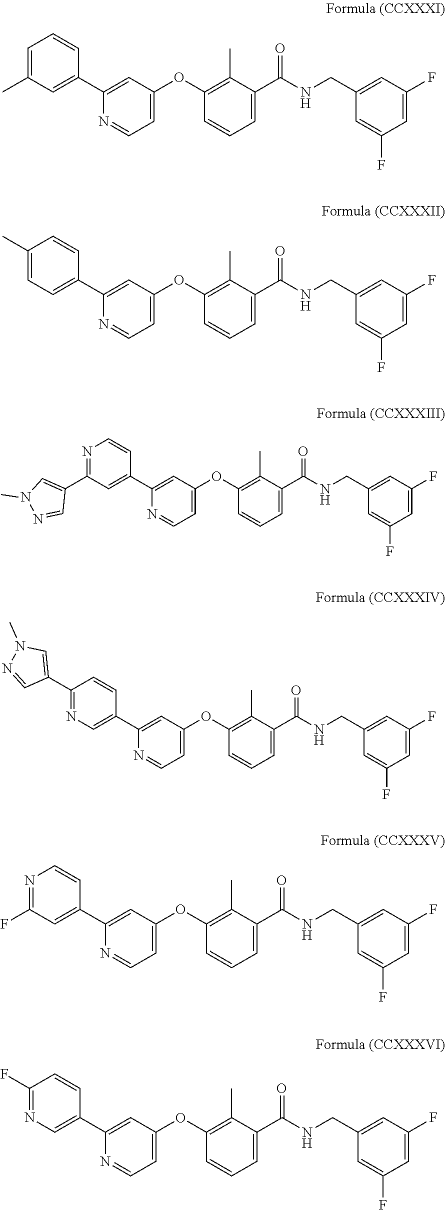

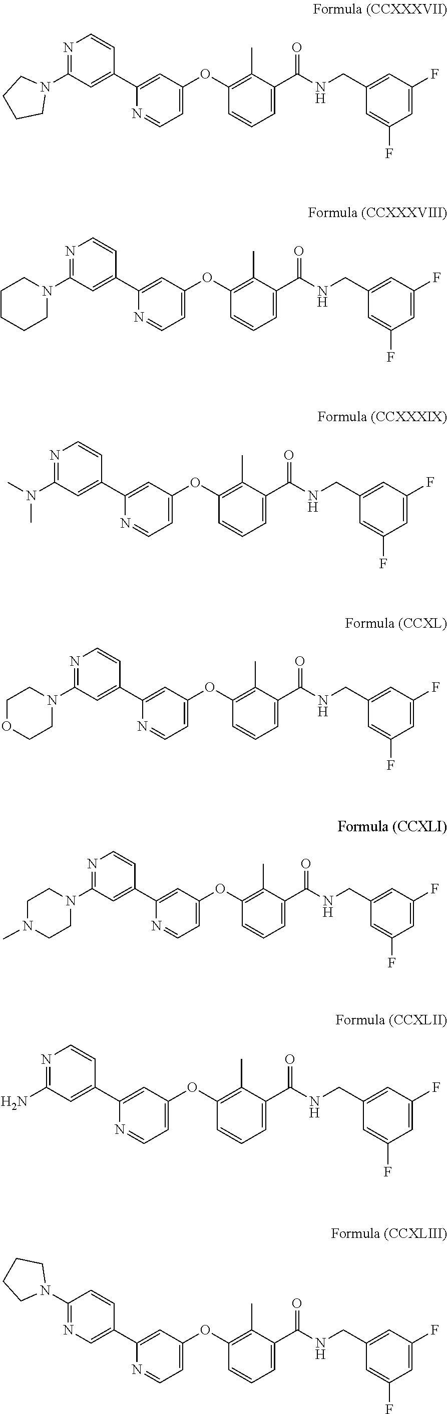

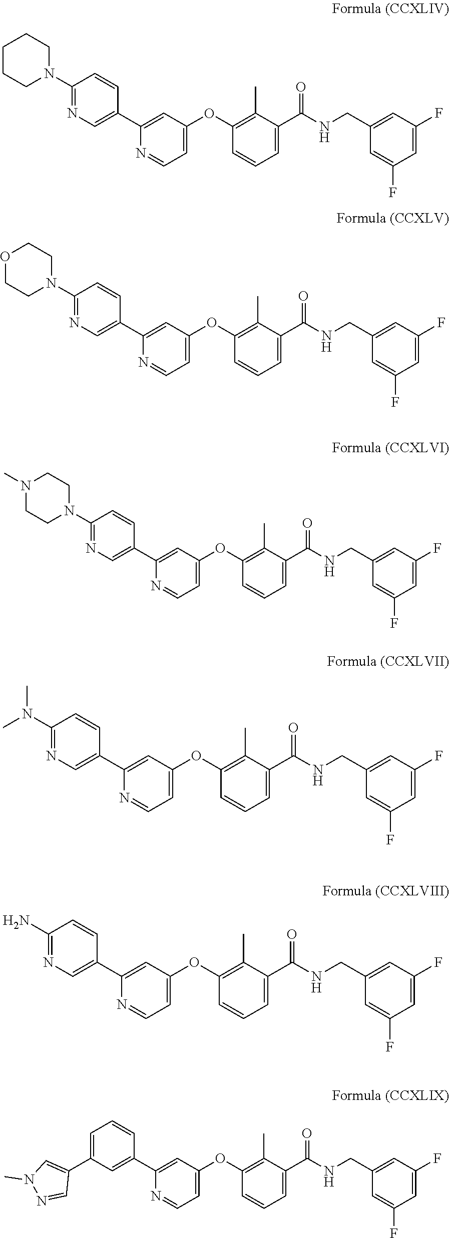

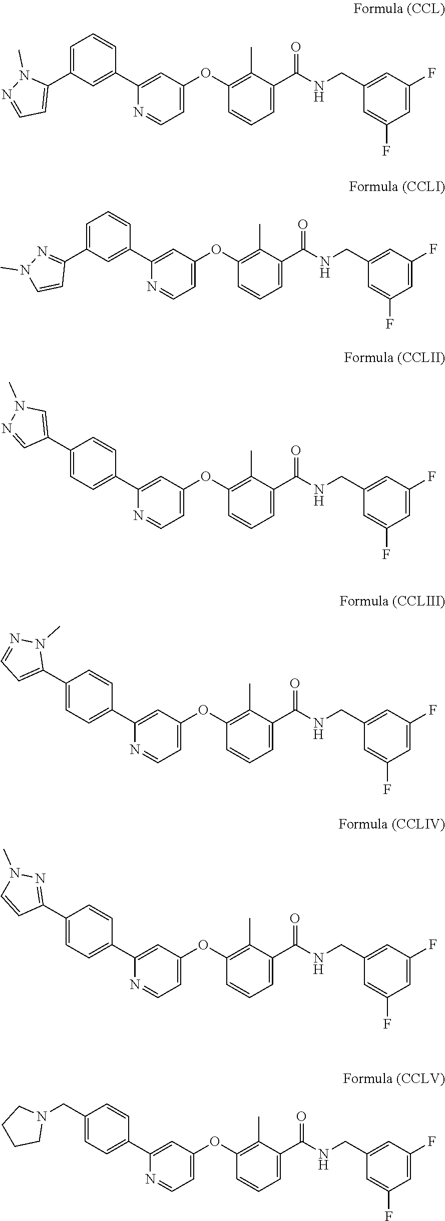

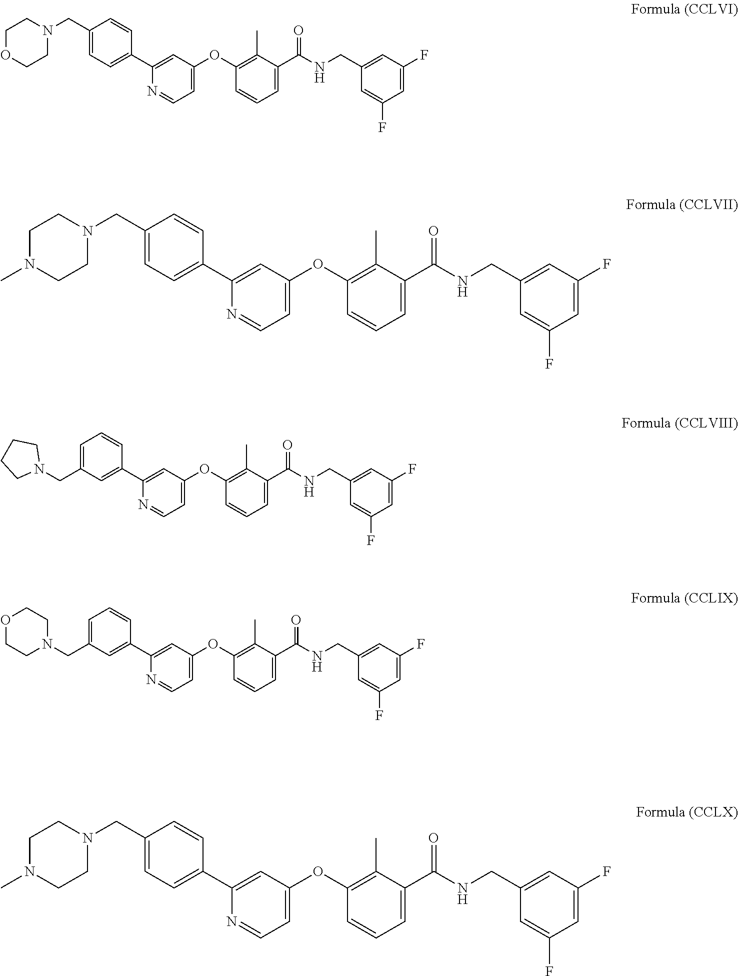

In a preferred embodiment of the present invention, the compound (C) according to general formula (VII-a) is a compound chosen among those of formulae (CCLXI) to (CCLXVII) herein below:

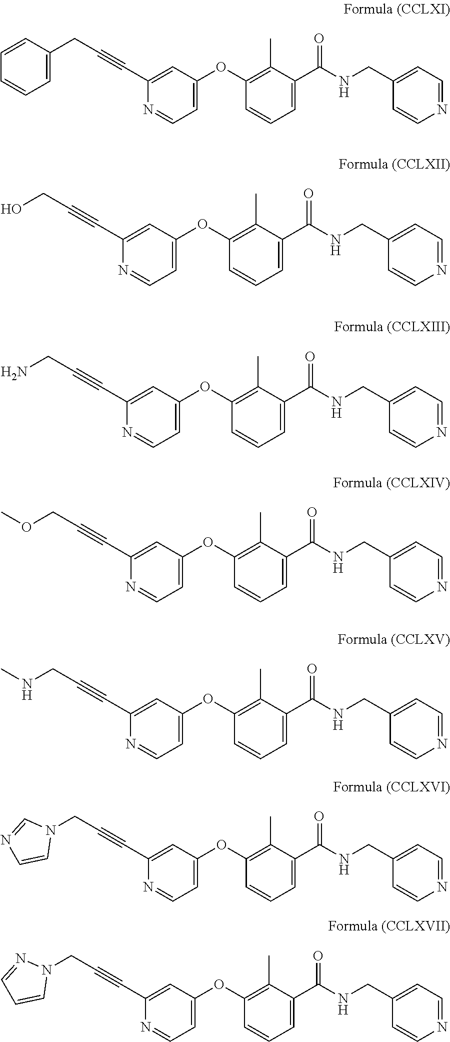

The present invention further relates to an in vitro method of inhibiting protein kinase activity which comprises contacting a protein kinase with a compound of formulae (I) to (VII) [compound (C), herein after], as defined above, or the N-oxide, pharmaceutically acceptable salt, pharmaceutically acceptable solvate, or stereoisomer thereof,

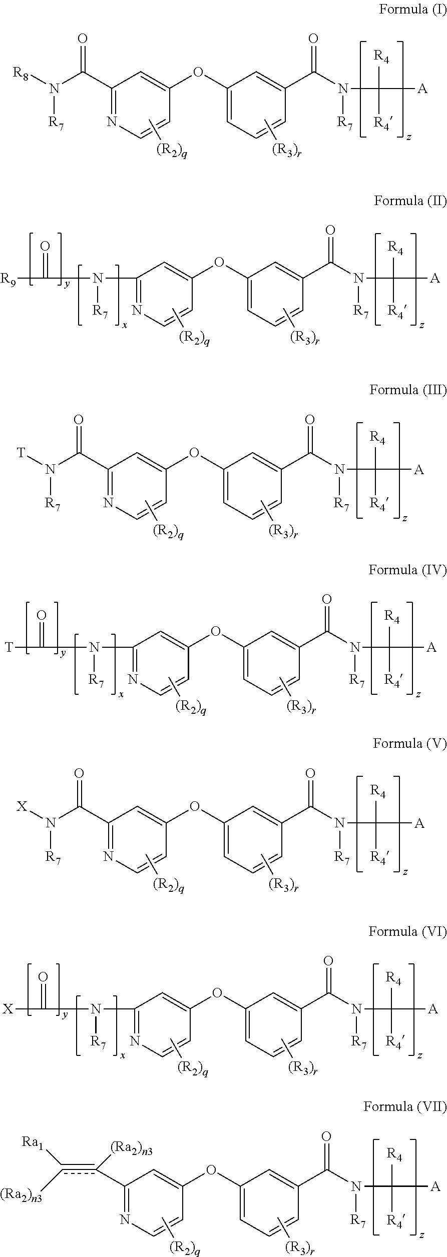

wherein:
each of A is independently selected from the group consisting of cycloalkyl, heterocyclyl, aryl, heteroaryl, and aralkyl, wherein said cycloalkyl, heterocyclyl, aryl, heteroaryl, and aralkyl are optionally substituted with one or more substituents independently selected from the group consisting of halo, NO₂, C₁₋₆ alkyl, C₂₋₄ alkenyl, C₂₋₄ alkynyl, cycloalkyl, heterocyclyl, aryl, heteroaryl, CF₃, CN, OR₁₁, SR₁₁, N(R₁₁)₂, OC(R₁₁)₂O, OC(R₁₁)₂C(R₁₁)₂O, S(O)R₁₂, SO₂R₁₂, SO₂N(R₁₁)₂, S(O)₃R₁₁, P(═O)(OR₁₁)₂, P(═O)(R₁₁)₂ NR₁₁COR₁₂, COR₁₁, C(O)OR₁₁, CON(R₁₁)₂, OC(O)R₁₁, and OCON(R₁₁)₂, and each optional alkyl, alkenyl, alkynyl, cycloalkyl, aryl, heterocyclyl, and heteroaryl substituents is further optionally substituted with halo, NO₂, C₁₋₆ alkyl, cycloalkyl, aryl, CF₃, N(R₁₁)₂, COR₁₁, CON(R₁₁)₂, OC(O)R₁₁, CN, or OR₁₁; and wherein each of R₁₁ and R₁₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, C₁₋₆ alkyl or aryl or heteroaryl amide, OR₃₁ or N(R₃₂)₂, wherein each of R₃₁ and R₃₂, independently from each other and at each occurrence, is selected from the group consisting of hydrogen and C₁₋₄ alkyl.
each of R₄ and R′₄, independently from each other and at each occurrence, are selected from hydrogen or C₁₋₆ alkyl, and z is an integer in the range from 0 to 2; with the proviso that when z=0, then A and R₇ may form together a saturated or unsaturated cyclic moiety;
each of R₇, independently from each other and at each occurrence is selected from hydrogen, C₁₋₆ alkyl, cycloalkyl, wherein said alkyl and cycloalkyl are optionally substituted by a halogen atom, CF₃, N(R₁₁)₂, CN, or OR₁₁; and wherein each of R₁₁, independently from each other and at each occurrence, is selected from the group consisting of hydrogen, C₁₋₆ alkyl, and CF₃;
each of R₃, independently from each other and at each occurrence, is selected from the group consisting of hydrogen, halo, C₁₋₆ alkyl, C₂₋₆ alkenyl, C₂₋₆ alkynyl, cycloalkyl, heterocyclyl, aryl, heteroaryl, aralkyl, CF₃, CN, OR₂₁, SR₂₁, N(R₂₁)₂, NC(O)R₂₁, NCON(R₂₁)₂, COR₂₁, C(O)OR₂₁, CON(R₂₁)₂, OC(O)R₂₁, OCON(R₂₁)₂, OC(R₂₁)₂O, and OC(R₂₁)₂C(R₂₂)₂O, wherein said alkyl, alkenyl, alkynyl, cycloalkyl, heterocyclyl, aryl, heteroaryl and aralkyl are optionally substituted with one or more substituents selected from halo, C₁₋₆ alkyl, CF₃, N(R₂₁)₂, CN, or OR₂₁; and wherein each of R₂₁ and R₂₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, and aralkyl, and wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, aryl, OR₃₁ or N(R₃₂)₂, wherein each of R₃₁ and R₃₂, independently from each other and at each occurrence, is selected from the group consisting of hydrogen and C₁₋₄ alkyl; each of r is an integer in the range from 0 to 3; with the proviso that when R₃═NR₂₁, and R₇═H, then R₃ and NR₇ may form together a saturated or unsaturated cyclic moiety;
each of R₂, independently from each other and at each occurrence, is selected from the group consisting of hydrogen, halo, C₁₋₆ alkyl, C₂₋₆ alkenyl, C₂₋₆ alkynyl, cycloalkyl, heterocyclyl, aryl, and heteroaryl, CF₃, CN, NO₂, OR₂₁, SR₂₁, N(R₂₁)₂, COR₂₁, C(O)OR₂₁, CON(R₂₁)₂, OC(O)R₂₁, OCON(R₂₁)₂, NC(O)R₂₁, NCON(R₂₁)₂, OC(R₂₁)₂O and OC(R₂₁)₂C(R₂₂)₂O, wherein said alkyl, alkenyl, alkynyl, cycloalkyl, heterocyclyl, aryl, and heteroaryl are optionally substituted with one or more substituents selected from halo, C₁₋₆ alkyl, C₂₋₆ alkenyl, C₂₋₆ alkynyl, cycloalkyl, heterocyclyl, CF₃, COR₂₁, CON(R₂₁)₂, C(O)OR₂₁, N(R₂₁)₂, CN, or OR₂₁, and each optional alkyl, alkenyl, alkynyl, cycloalkyl, heterocyclyl, aryl or heteroaryl substituent is further optionally substituted with heterocyclyl, N(R₁₁)₂, or OR₁₁; and wherein each of R₂₁ and R₂₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, and aralkyl, and wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, aryl, OR₃₁ or N(R₃₂)₂, wherein each of R₃₁ and R₃₂, independently from each other and at each occurrence, is selected from the group consisting of hydrogen and C₁₋₄ alkyl; each of q is an integer in the range from 0 to 2;
each of x and y are independently integers equal to 0 or 1;
R₈ is independently selected from the group consisting of C₆₋₁₂ alkyl, C₂₋₆ alkenyl, C₂₋₆ alkynyl, cycloalkyl and heterocyclyl, wherein said alkyl, alkenyl, alkynyl, cycloalkyl, and heterocyclyl are optionally substituted by a halogen atom, an aryl group, an aralkyl group, CF₃, N(R₁₁)₂, CN, or OR₁₁; and wherein each of R₁₁, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, alkyl or aryl or heteroaryl amide, OR₃₁ or N(R₃₂)₂, wherein each of R₃₁ and R₃₂, independently from each other and at each occurrence, is selected from the group consisting of hydrogen and C₁₋₄ alkyl;
R₉ is selected from the group consisting of hydrogen, C₁₋₆ alkyl, C₂₋₆ alkenyl, C₂₋₆ alkynyl, cycloalkyl, heterocyclyl, N(R₁₁)₂ and CN, wherein said alkyl, alkenyl, alkynyl, cycloalkyl, and heterocyclyl are optionally substituted by a halogen atom, an aryl group, an aralkyl group, an heterocyclyl group, CF₃, N(R₁₁)₂, CN, or OR₁₁; and wherein each of R₁₁, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, heteroaryl optionally substituted with a C₁₋₄ alkyl, aryl, heteroaryl, and aralkyl substituents are optionally substituted with halo, C₁₋₆ alkyl, cycloalkyl, heterocyclyl, alkyl or aryl or heteroaryl amide, OR₃₁ or N(R₃₂)₂, wherein each of R₃₁ and R₃₂, independently from each other and at each occurrence, is selected from the group consisting of hydrogen and C₁₋₄ alkyl, with the proviso that if x=1 and y=0, R₉ is different from heterocyclyl, and from C₁₋₆ alkyl wherein said alkyl is optionally substituted with heterocyclyl; and with the proviso that if x=0 and y=0, R₉ is different from hydrogen, and C₁₋₆ alkyl, wherein said alkyl is optionally substituted with heterocyclyl and N(R₁₁)₂; with the proviso that when x=0 and y=0, R₉ and R₂ may form together a saturated or an unsaturated cyclic moiety; with the proviso that when x=0 and y=0 and when R₉ and R₂ form together a saturated or an unsaturated cyclic moiety, R₉ is NR₁₁; with the proviso that when x=1 and y=1, R₉ is different from N(R₁₁)₂; and with the proviso that when x=0, y=0 and z=0, R₉ is different from pyrrole.
each of T is independently the moiety of formula (T-a) herein below:

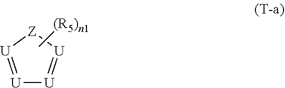

wherein:
each of U, independently from each other and at each occurrence, is selected from the group consisting of C, C-halo, C—R, and N; wherein R is selected from hydrogen, OR₁₁, N(R₁₁)₂, a C₁₋₆ alkyl or a cycloalkyl which are optionally substituted by a halogen atom, an aryl group or an aralkyl group, wherein each of R₁₁, independently from each other and at each occurrence, is selected from the group consisting of hydrogen or C₁₋₄ alkyl; with the proviso that at least one U is different from N;
each of Z, independently from each other and at each occurrence is selected from C(R)₂, O, S and NR₇, wherein R, independently from each other and at each occurrence is selected from hydrogen or an C₁₋₆ alkyl which is optionally substituted by a halogen atom, an aryl group or an aralkyl group, wherein R is selected from the group consisting of hydrogen, C₁₋₆ alkyl, C₁₋₆ alkenyl, cycloalkyl, heterocyclyl, aryl, aralkyl and CF₃;
each of R₅, independently from each other and at each occurrence is selected from the group consisting of C₁₋₆ alkyl, C₂₋₆ alkenyl, C₂₋₆ alkynyl, cycloalkyl, heterocyclyl, aryl, heteroaryl, aralkyl, halo, CF₃, OR₁₁, SR₁₁, N(R₁₁)₂, COOR₁₁, CO(R₁₁)₂, CON(R₁₁)₂, and each optional alkyl, alkenyl, alkynyl, cycloalkyl, aryl, heterocyclyl, heteroaryl, aralkyl substituent is further optionally substituted with halo, C₁₋₆ alkyl, cycloalkyl, aryl, heterocyclyl, N(R₁₁)₂, CN, OR₁₁, C(═O)OR₁₁, P(═O)(OR₁₁)₂, P(═O)(R₁₁)₂, CN or CF₃ and wherein each of R₁₁, independently from each other and at each occurrence, is selected from the group consisting of hydrogen, C₁₋₆ alkyl, cycloalkyl, and heterocyclyl; each of n1 is an integer in the range from 0 to 2;
each of X is independently the moiety of formula (X-a) herein below:

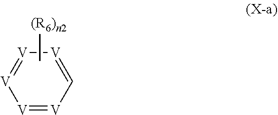

wherein:
each of V, independently from each other and at each occurrence, is selected from the group consisting of C, C-halo, C—R, and N; wherein R is selected from hydrogen, OR₁₁, N(R₁₁)₂, a C₁₋₆ alkyl or a cycloalkyl which are optionally substituted by a halogen atom, an aryl group or an aralkyl group, wherein each of R₁₁, independently from each other and at each occurrence, is selected from hydrogen or C₁₋₄ alkyl;
each of R₆, independently from each other and at each occurrence is selected from the group consisting of C₁₋₆ alkyl, C₂₋₆ alkenyl, C₂₋₆ alkynyl, cycloalkyl, heterocyclyl, aryl, heteroaryl, aralkyl, halo, CF₃, OR₁₁, SR₁₁, N(R₁₁)₂, COOR₁₁, CO(R₁₁)₂, CON(R₁₁)₂, and each optional alkyl, alkenyl, alkynyl, cycloalkyl, aryl, heterocyclyl, heteroaryl, aralkyl substituent is further optionally substituted with halo, C₁₋₆ alkyl, cycloalkyl, aryl, heterocyclyl, N(R₁₁)₂, CN, OR₁₁, C(═O)OR₁₁, P(═O)(OR₁₁)₂, P(═O)(R₁₁)₂, CN or CF₃ and wherein each of R₁₁, independently from each other and at each occurrence, is selected from the group consisting of hydrogen, C₁₋₆ alkyl, cycloalkyl, and heterocyclyl; each of n2 is an integer in the range from 0 to 4;
the dash bond represents an optional triple bond;
Rₐ₁ is independently selected from the group consisting of hydrogen, C₁₋₆ alkyl, cycloalkyl, heterocyclyl, aryl, heteroaryl, aralkyl, CF₃, CN, OR₁₁, SR₁₁, N(R₁₁)₂, COR₁₁, C(O)OR₁₁, wherein said alkyl cycloalkyl, heterocyclyl, aryl, heteroaryl and aralkyl are optionally substituted by halo, NO₂, C₁₋₆ alkyl, C₂₋₆ alkenyl, C₂₋₆ alkynyl, cycloalkyl, heterocyclyl, phenyl, heteroaryl, CF₃, CN, OR₁₁, SR₁₁, N(R₁₁)₂, COR₁₁, and C(O)OR₁₁, and each optional alkyl, alkenyl, cycloalkyl, phenyl, heterocyclyl, heteroaryl substituent is further optionally substituted with halo, NO₂, C₁₋₆ alkyl, cycloalkyl, phenyl, N(R₁₁)₂, CN, or OR₁₁; and wherein each of R₁₁ is selected from the group consisting of hydrogen, C₁₋₆ alkyl, cycloalkyl, heterocyclyl, phenyl, heteroaryl, and aralkyl, wherein said alkyl, cycloalkyl, heterocyclyl, phenyl, heteroaryl, and aralkyl substituents are optionally substituted with halo, C₁₋₆ alkyl, cycloalkyl or heterocyclyl.
each of Rₐ₂, independently from each other and at each occurrence, is selected from the group consisting of hydrogen, C₁₋₆ alkyl, cycloalkyl, heterocyclyl, aryl, heteroaryl, aralkyl, CF₃, CN, OR₁₁, SR₁₁, N(R₁₁)₂, COR₁₁, C(O)OR₁₁, wherein said alkyl, alkenyl, alkynyl, cycloalkyl, heterocyclyl, aryl, heteroaryl and aralkyl are optionally substituted by halo, NO₂, C₁₋₆ alkyl, C₂₋₆ alkenyl, C₂₋₆ alkynyl, cycloalkyl, heterocyclyl, phenyl, heteroaryl, CF₃, CN, OR₁₁, SR₁₁, N(R₁₁)₂, COR₁₁, and C(O)OR₁₁, and each optional alkyl, alkenyl, cycloalkyl, phenyl, heterocyclyl, heteroaryl substituent is further optionally substituted with halo, NO₂, C₁₋₆ alkyl, cycloalkyl, phenyl, N(R₁₁)₂, CN, or OR₁₁; and wherein each of R₁₁ is selected from the group consisting of hydrogen, C₁₋₆ alkyl, cycloalkyl, heterocyclyl, phenyl, heteroaryl, and aralkyl, wherein said alkyl, cycloalkyl, heterocyclyl, phenyl, heteroaryl, and aralkyl substituents are optionally substituted with halo, C₁₋₆ alkyl, cycloalkyl or heterocyclyl; and wherein n3 is an integer equal to 0 or 1; with the proviso that when the dash bond represents a triple bond, n3 is 0;
   wherein said cycloalkyl is a monocyclic, bicyclic or tricyclic ring system of 3-6 ring members per ring; said heterocyclyl is a saturated, partially saturated or completely saturated monocycle, bicycle or tricycle containing 3 to 12 carbon atoms and 1 or 2 heteroatoms independently selected from O or N; said aryl is phenyl, naphthyl or anthracenyl optionally carbocyclic fused with a cycloalkyl or heterocyclyl of 5-7 ring members; said heteroaryl is a monocyclic ring structure containing 5 or 6 ring atoms, or a bicyclic aromatic group having 8 to 10 atoms, containing 1-3 heteroatoms independently selected from O or N.

It is further understood that all definitions and preferences as described for compound (C) above equally apply for this embodiment and all further embodiments, as described below.

As used in the foregoing and hereinafter, the following definitions apply unless otherwise noted.

The term halo-alone or in combination means all halogens, that is, chloro (Cl), bromo (Br), fluoro (F), iodo (I).

The term alkyl-alone or in combination means an alkane-derived radical containing from 1 to 15 carbon atoms, unless otherwise specified, for example CF-G alkyl defines a straight or branched alkyl radical having from F to G carbon atoms, e.g. C₁₋₄ alkyl defines a straight or branched alkyl radical having from 1 to 4 carbon atoms such as for example methyl, ethyl, 1-propyl, 2-propyl, I-butyl, 2-butyl, 2-methyl-1-propyl. An alkyl group may be a straight chain alkyl or branched alkyl. Preferably, straight or branched alkyl groups containing from 1-10, more preferably 1 to 8, even more preferably 1-6 and most preferably 1-4, carbon atoms, such as methyl, ethyl, propyl, isopropyl, butyl, t-butyl and the like. Alkyl also includes a straight chain or branched alkyl group that contains or is interrupted by a cycloalkyl portion. The straight chain or branched alkyl group is attached at any available point to produce a stable compound. Examples of this include, but are not limited to, 4-(isopropyl)-cyclohexylethyl or 2-methyl-cyclopropylpentyl.

The term alkenyl-alone or in combination means a straight or branched hydrocarbon containing 2-15 more preferably 2-10, even more preferably 2-8, most preferably 2-4, carbon atoms, unless otherwise specified and at least one, preferably 1-3, more preferably 1-2, most preferably one, carbon to carbon double bond. Examples of alkenyl groups include ethenyl, propenyl, isopropenyl, butenyl, cyclohexenyl, cyclohexenylalkyl and the like. Alkenyl also includes a straight chain or branched alkenyl group that contains or is interrupted by a cycloalkyl portion. Carbon to carbon double bonds may be either contained within a cycloalkyl portion, with the exception of cyclopropyl, or within a straight chain or branched portion.

The term alkynyl-alone or in combination means a straight or branched hydrocarbon containing 2-15 more preferably 2-10, even more preferably 2-8, most preferably 2-4, carbon atoms containing at least one, preferably one, carbon to carbon triple bond. Examples of alkynyl groups include ethynyl, propynyl, butynyl and the like.

The term aryl-alone or in combination means phenyl, naphthyl or anthracenyl optionally carbocyclic fused with a cycloalkyl or heterocyclyl of preferably 5-7, more preferably 5-6, ring members and/or optionally substituted with 1 to 5 groups or substituent. An aryl may be optionally substituted whereby the substituent is attached at one point to the aryl or whereby the substituent is attached at two points to the aryl to form a bicyclic system e.g. benzodioxole, benzodioxan, benzimidazole.

The term heteroaryl-alone or in combination means a monocyclic aromatic ring structure containing 5 or 6 ring atoms, or a bicyclic aromatic group having 8 to 10 atoms, containing 1-3, heteroatoms independently selected from the group O, S, and N, and optionally substituted with 1 to 5 groups or substituents. Heteroaryl is also intended to include oxidized S or N, such as sulfinyl, sulfonyl and N-oxide of a tertiary ring nitrogen. A carbon or nitrogen atom is the point of attachment of the heteroaryl ring structure such that a stable aromatic ring is retained. More specifically the term heteroaryl includes, but is not limited to, pyridyl, furanyl, thiophenyl, thiazolyl, isothiazolyl, triazolyl, imidazolyl, isoxazolyl, pyrrolyl, pyrazolyl, pyrimidinyl, benzofuranyl, isobenzofuranyl, benzothiazolyl, benzoisothiazolyl, benzotriazolyl, indolyl, isoindolyl, benzoxazolyl, quinolyl, isoquinolyl, benzimidazolyl, benzisoxazolyl, benzothiophenyl, dibenzofuran, and benzodiazepin-2-one-5-yl, and the like.

The term heterocyclyl-alone or in combination is intended to denote a saturated, partially unsaturated or completely unsaturated monocycle, bicycle, or tricycle having 3 to 12 carbon atoms and containing 1 or 2 heteroatoms each independently selected from O, S, P or N, and are optionally benzo fused or fused heteroaryl of 5-6 ring members and/or are optionally substituted as in the case of cycloalkyl. Heterocycyl is also intended to include oxidized S or N, such as sulfinyl, sulfonyl and N-oxide of a tertiary ring nitrogen. The point of attachment is at a carbon or nitrogen atom. In each case the heterocyclyl may be condensed with an aryl to form a bicyclic ring system.

The term cycloalkyl refers to a cyclic or polycyclic alkyl group containing 3 to 7 carbon atoms. Preferably, cycloalkyl groups are monocyclic, bicyclic or tricyclic ring systems of 3-6, ring members per ring, such as cyclopropyl, cyclopentyl, cyclohexyl, adamantyl and the like.

The term aralkyl refers to organic compounds containing an aromatic nucleus to which an alkyl radical is bonded. These alkyl radicals include methyl, ethyl, propyl, butyl, octyl, etc. radicals. The term aralkyl is thus seen to include aralkyl hydrocarbons such as the alkyl benzenes, and the various alkyl naphthalenes. From this definition of the term aralkyl compound it is seen that the term includes compounds such as benzyl, the three isomeric xylyls, the two isomeric trimethyl benzenes, ethyl benzene, p-methyl biphenyl, a-methyl naphthalene, etc.

The present invention further relates to a pharmaceutical composition comprising a carrier, and as active ingredient an effective amount of a compound (C) formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, and as defined in any one of the embodiments presented herein.

The present invention relates to a compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-c), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2) as specified herein, and as defined in any one of the embodiments presented herein, for use as a medicament.

The present invention relates to a compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, and as defined in any one of the embodiments presented herein, for use in the treatment of a disease selected from from cancer, metabolic disorders (such as diabetes), inflammatory and autoimmune disorders (such as inflammatory bowel diseases, e.g. Crohn's disease and ulcerative colitis, inflammatory pulmonary diseases, rheumatoid arthritis, lupus nephritis, systemic lupus erythematosus and psoriasis and psoriasis arthritis), neurological disorders (such as Alzheimer's disease, Parkinson's disease, multiple sclerosis, Charcot-Marie-Tooth neuropathy, amyotrophic lateral sclerosis and epilepsy), atherosclerosis and cardiovascular diseases, Sjogren Syndrome, renal allograft rejection, viral induced diseases, circulatory diseases, bone osteolysis and osteoporosis, osteoarthritis, sarcopenia, Langerhans cell histiocytosis, spinal cord injury, endometriosis, asthma and allergic asthma, eye diseases (such as retinopathies, age-related macular degeneration and uveitis) chronic and neuropathic pain, and fibro-proliferative diseases.

The present invention further relates to a method of inhibiting protein kinase activity in a warm-blooded animal said method comprising the administration to an animal in need thereof, of a kinase-inhibitory effective amount of a compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, and according to any one of the embodiments presented herein.

The present invention further relates to a method of inhibiting protein kinase activity in a warm-blooded animal said method comprising the administration to an animal in need thereof, of a kinase-inhibitory effective amount of a compound (C) formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, and according to any one of the embodiments presented herein, wherein the protein kinase is selected from the group consisting of CSF1R, FLT3, Kit, PDGFRB (PDGFR beta), PDGFRA (PDGFR alfa), ABL1, ACVR1B (ALK4), AKT1 (PKB alpha), AMPK A1/B1/G1, AURKA (Aurora A), BTK, CDK1/cyclin B, CHEK1 (CHK1), CSNK1G2 (CK1 gamma 2), CSNK2A1 (CK2 alpha 1), DYRK3, EGFR (ErbB1), EPHA2, ERBB2 (HER2), FGFR₁, FRAP1 (mTOR), GSK3B (GSK3 beta), IGF1R, IKBKB (IKK beta), INSR, IRAK4, JAK3, KDR (VEGFR₂), LCK, MAP2K1 (MEK1), MAP4K4 (HGK), MAPK1 (ERK2), MAPK14 (p38 alpha), MAPK3 (ERK1), MAPK8 (JNK1), MARK2, MET (cMet), NEK1, PAK4, PHKG2, PIM1, PLK1, PRKACA (PKA), PRKCB1 (PKC beta I), ROCK1, RPS6KA3 (RSK2), RPS6KB1 (p70S6K), SRC, SYK, and TEK (Tie2). Preferably, the protein kinase is selected from the group consisting of CSF1R, FLT3, Kit, PDGFRB (PDGFR beta), PDGFRA (PDGFR alpha).

The present invention further relates to a method of treating a disease selected from cancer, metabolic disorders (such as diabetes), inflammatory and autoimmune disorders (such as inflammatory bowel diseases, e.g. Crohn's disease and ulcerative colitis, inflammatory pulmonary diseases, rheumatoid arthritis, lupus nephritis, systemic lupus erythematosus and psoriasis and psoriasis arthritis), neurological disorders (such as Alzheimer's disease, Parkinson's disease, multiple sclerosis, Charcot-Marie-Tooth neuropathy, amyotrophic lateral sclerosis and epilepsy), atherosclerosis and cardiovascular diseases, Sjogren Syndrome, renal allograft rejection, viral induced diseases, circulatory diseases, bone osteolysis and osteoporosis, osteoarthritis, sarcopenia, Langerhans cell histiocytosis, spinal cord injury, endometriosis, asthma and allergic asthma, eye diseases (such as retinopathies, age-related macular degeneration and uveitis) chronic and neuropathic pain, and fibro-proliferative diseases, in a warm-blooded animal said method comprising the administration to an animal in need thereof of an effective amount of a compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, and according to any one of the embodiments presented herein.

It should be noted that the radical positions on any molecular moiety used in the definitions may be anywhere on such moiety as long as it is chemically stable.

Radicals used in the definitions of the variables include all possible isomers unless otherwise indicated. For instance pyridyl includes 2-pyridyl, 3-pyridyl and 4-pyridyl; pentyl includes 1-pentyl, 2-pentyl and 3-pentyl.

When any variable occurs more than one time in any constituent, each definition is independent. Whenever used hereinafter, the term “compounds (C) of formulae (I) to (VII)”, or “the present compounds” or similar terms, it is meant to include all the compounds (C) of formulae (I) to (VII), N-oxides, addition salts, and stereochemically isomeric forms. One embodiment comprises the compounds (C) of formulae (I) to (VII), or any subgroup of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), specified herein, as well as the N-oxides, salts, as the possible stereoisomeric forms thereof. Another embodiment comprises the compounds (C) of formula formulae (I) to (VII), or any subgroup of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), specified herein, as well as the salts as the possible stereoisomeric forms thereof.

The compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, have several centers of chirality and exist as stereochemically isomeric forms. The term “stereochemically isomeric forms” as used herein defines all the possible compounds made up of the same atoms bonded by the same sequence of bonds but having different three-dimensional structures which are not interchangeable, which the compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, may possess.

Unless otherwise mentioned or indicated, the chemical designation of a compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, encompasses the mixture of all possible stereochemically isomeric forms, which said compound may possess. Said mixture may contain all diastereomers and/or enantiomers of the basic molecular structure of said compound. All stereochemically isomeric forms of the compounds of the present invention both in pure form or mixed with each other are intended to be embraced within the scope of the present invention.

Pure stereoisomeric forms of the compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, and intermediates as mentioned herein are defined as isomers substantially free of other enantiomeric or diastereomeric forms of the same basic molecular structure of said compounds or intermediates. In particular, the term “stereoisomerically pure” concerns compounds or intermediates having a stereoisomeric excess of at least 80% (i.e. minimum 90% of one isomer and maximum 10% of the other possible isomers) up to a stereoisomeric excess of 100% (i.e. 100% of one isomer and none of the other), more in particular, compounds or intermediates having a stereoisomeric excess of 90% up to 100%, even more in particular having a stereoisomeric excess of 94% up to 100% and most in particular having a stereoisomeric excess of 97% up to 100%. The terms “enantiomerically pure” and “diastereomerically pure” should be understood in a similar way, but then having regard to the enantiomeric excess, and the diastereomeric excess, respectively, of the mixture in question.

Pure stereoisomeric forms of the compounds and intermediates of this invention may be obtained by the application procedures known in the art. For instance, enantiomers may be separated from each other by the selective crystallization of their diastereomeric salts with optically active acids or bases. Examples thereof are tartaric acid, dibenzoyltartaric acid, ditoluoyltartaric acid and camphorsulfonic acid. Alternatively, enantiomers may be separated by chromatographic techniques using chiral stationary phases. Said pure stereochemically isomeric forms may also be derived from the corresponding pure stereochemically isomeric forms of the appropriate starting materials, provided that the reaction occurs stereospecifically. Preferably, if a specific stereoisomer is desired, said compound will be synthesized by stereospecific methods of preparation. These methods will advantageously employ enantiomerically pure starting materials.

The diastereomeric racemates of the compounds (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, can be obtained separately by conventional methods. Appropriate physical separation methods that may advantageously be employed are, for example, selective crystallization and chromatography, e.g. column chromatography.

For some of the compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, N-oxides, salts, solvates, and the intermediates used in the preparation thereof, the absolute stereochemical configuration was not experimentally determined.

A person skilled in the art is able to determine the absolute configuration of such compounds using art-known methods such as, for example, X-ray diffraction.

The present invention is also intended to include all isotopes of atoms occurring on the present to a compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein. Isotopes include those atoms having the same atomic number but different mass numbers. By way of general example and without limitation, isotopes of hydrogen include tritium and deuterium. Isotopes of carbon include C-13 and C-14.

For therapeutic use, salts of the compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, are those wherein the counter-ion is pharmaceutically acceptable, which salts can be referred to as pharmaceutically acceptable acid and base addition salts. However, salts of acids and bases that are non-pharmaceutically acceptable may also find use, for example, in the preparation or purification of a pharmaceutically acceptable compound. All salts, whether pharmaceutically acceptable or not, are included within the ambit of the present invention.

The pharmaceutically acceptable acid and base addition salts as mentioned hereinabove are meant to comprise the therapeutically active non-toxic acid and base addition salt forms that the compounds (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), or formula (VII-a-2), as specified herein, are able to form. The pharmaceutically acceptable acid addition salts can conveniently be obtained by treating the base form with such appropriate acid in an anion form. Appropriate anions comprise, for example, trifluoroacetate, acetate, benzenesulfonate, benzoate, bicarbonate, bitartrate, bromide, calcium edetate, camsyiate, carbonate, chloride, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, malate, maleate, mandelate, mesylate, methylbromide, methylnitrate, methylsulfate, mucate, napsylate, nitrate, pamoate (embonate), pantothenate, phosphate/diphosphate, polygalacturonate, salicylate, stearate, subacetate, succinate, sulfate, tannate, tartrate, teoclate, triethiodide, and the like. The counterion of choice can be introduced using ion exchange resins. Conversely said salt forms can be converted by treatment with an appropriate base into the free base form.

The compounds (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), or formula (VII-a-2), as specified herein, containing an acidic proton may also be converted into their nontoxic metal or amine addition salt forms by treatment with appropriate organic and inorganic bases in a cation form. Appropriate basic salts comprise those formed with organic cations such as benzathine, chloroprocaine, choline, diethanolamine, ethylenediamine, meglumine, procaine, and the like; and those formed with metallic cations such as aluminum, calcium, lithium, magnesium, potassium, sodium, zinc, and the like. Conversely said salt forms can be converted by treatment with an appropriate acid into the free form.

The term addition salt as used hereinabove also comprises the solvates which the compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, as well as the salts thereof, are able to form. Such solvates are for example hydrates, alcoholates and the like.

The N-oxide forms of the present compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, are meant to comprise the compounds of formula (I) wherein one or several nitrogen atoms are oxidized to the so-called N-oxide.

It will be appreciated that the compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, may have metal binding, chelating, complex forming properties and therefore may exist as metal complexes or metal chelates. Such metalated derivatives of the compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, are intended to be included within the scope of the present invention.

Some of the compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, may also exist in their tautomeric form. Such forms although not explicitly indicated in the above formula are intended to be included within the scope of the present invention.

In a further aspect, the present invention concerns a pharmaceutical composition comprising a therapeutically effective amount of a compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, and a pharmaceutically acceptable carrier. A therapeutically effective amount in this context is an amount sufficient to prophylactically act against, to stabilize or reduce illnesses mediated by protein kinases in ill subjects or subjects being at risk of being ill, in particular a protein kinase selected from the group consisting of CSF1R, FLT3, Kit, PDGFRB (PDGFR beta), PDGFRA (PDGFR alfa), ABL1, ACVR1B (ALK4), AKT1 (PKB alpha), AMPK A1/B1/G1, AURKA (Aurora A), BTK, CDK1/cyclin B, CHEK1 (CHK1), CSNK1G2 (CK1 gamma 2), CSNK2A1 (CK2 alpha 1), DYRK3, EGFR (ErbB1), EPHA2, ERBB2 (HER2), FGFR₁, FRAP1 (mTOR), GSK3B (GSK3 beta), IGF1R, IKBKB (IKK beta), INSR, IRAK4, JAK3, KDR (VEGFR₂), LCK, MAP2K1 (MEK1), MAP4K4 (HGK), MAPK1 (ERK2), MAPK14 (p38 alpha), MAPK3 (ERK1), MAPK8 (JNK1), MARK2, MET (cMet), NEK1, PAK4, PHKG2, PIM1, PLK1, PRKACA (PKA), PRKCB1 (PKC beta I), ROCK1, RPS6KA3 (RSK2), RPS6KB1 (p70S6K), SRC, SYK, and TEK (Tie2). Preferably, the protein kinase is selected from the group consisting of CSF1R, FLT3, Kit, PDGFRB (PDGFR beta), PDGFRA (PDGFR alpha).

Examples of illnesses mediated by protein kinases include in particular of illnesses mediated by protein kinases include in particular cancer, metabolic disorders (such as diabetes), inflammatory and autoimmune disorders (such as inflammatory bowel diseases, e.g. Crohn's disease and ulcerative colitis, inflammatory pulmonary diseases, rheumatoid arthritis, lupus nephritis, systemic lupus erythematosus and psoriasis and psoriasis arthritis), neurological disorders (such as Alzheimer's disease, Parkinson's disease, multiple sclerosis, Charcot-Marie-Tooth neuropathy, amyotrophic lateral sclerosis and epilepsy), atherosclerosis and cardiovascular diseases, Sjogren Syndrome, renal allograft rejection, viral induced diseases, circulatory diseases, bone osteolysis and osteoporosis, osteoarthritis, sarcopenia, Langerhans cell histiocytosis, spinal cord injury, endometriosis, asthma and allergic asthma, eye diseases (such as retinopathies, age-related macular degeneration and uveitis) chronic and neuropathic pain, and fibro-proliferative diseases.

In still a further aspect, this invention relates to a process of preparing a pharmaceutical composition as specified herein, which comprises intimately mixing a pharmaceutically acceptable carrier with a therapeutically effective amount of a compound (C) of formulae (I) to (VII), as specified herein, or of a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein.

Therefore, the compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, may be formulated into various pharmaceutical forms for administration purposes. As appropriate compositions there may be cited all compositions usually employed for systemically administering drugs. To prepare the pharmaceutical compositions of this invention, an effective amount of the particular compound, optionally in addition salt form or metal complex, as the active ingredient is combined in intimate admixture with a pharmaceutically acceptable carrier, which carrier may take a wide variety of forms depending on the form of preparation desired for administration. These pharmaceutical compositions are desirable in unitary dosage form suitable, particularly, for administration orally, rectally, percutaneously, or by parenteral injection. For example, in preparing the compositions in oral dosage form, any of the usual pharmaceutical media may be employed such as, for example, water, glycols, oils, alcohols and the like in the case of oral liquid preparations such as suspensions, syrups, elixirs, emulsions and solutions; or solid carriers such as starches, sugars, kaolin, lubricants, binders, disintegrating agents and the like in the case of powders, pills, capsules, and tablets.

Because of their ease in administration, tablets and capsules represent the most advantageous oral dosage unit forms, in which case solid pharmaceutical carriers are obviously employed. For parenteral compositions, the carrier will usually comprise sterile water, at least in large part, though other ingredients, for example, to aid solubility, may be included. Injectable solutions, for example, may be prepared in which the carrier comprises saline solution, glucose solution or a mixture of saline and glucose solution. Injectable suspensions may also be prepared in which case appropriate liquid carriers, suspending agents and the like may be employed. Also included are solid form preparations, which are intended to be converted, shortly before use, to liquid form preparations. In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin.

The compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, of the present invention may also be administered via oral inhalation or insufflation by means of methods and formulations employed in the art for administration via this way. Thus, in general the compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, may be administered to the lungs in the form of a solution, a suspension or a dry powder, a solution being preferred. Any system developed for the delivery of solutions, suspensions or dry powders via oral inhalation or insufflation are suitable for the administration of the present compounds.

Thus, the present invention also provides a pharmaceutical composition adapted for administration by inhalation or insufflation through the mouth comprising a compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, and a pharmaceutically acceptable carrier. Preferably, the compounds of the present invention are administered via inhalation of a solution in nebulized or aerosolized doses.

It is especially advantageous to formulate the aforementioned pharmaceutical compositions in unit dosage form for ease of administration and uniformity of dosage. Unit dosage form as used herein refers to physically discrete units suitable as unitary dosages, each unit containing a predetermined quantity of active ingredient calculated to produce the desired therapeutic effect in association with the required pharmaceutical carrier. Examples of such unit dosage forms are tablets (including scored or coated tablets), capsules, pills, suppositories, powder packets, wafers, injectable solutions or suspensions and the like, and segregated multiples thereof.

The compound (C) of formulae (I) to (VII) as specified herein, or a compound of any of the subgroups of compounds of formula (II-a) to (II-b), (III-a), (IV-a) to (IV-c), (VI-a) to (VI-c), (VII-a), (VII-b), (II-a-1) to (II-c-1), (IV-a-1) to (IV-c-1), (VI-a-1) to (VI-c-1), (VII-a-1) to (VII-b-1), (II-a-2) to (II-c-2), (IV-a-2-1) to (IV-c-2-4), (IV-a-2) to (VI-c-2), or formula (VII-a-2), as specified herein, show kinase inhibition properties. Illnesses and diseases treatable using the compounds and methods of the present invention include protein kinase mediated diseases like like cancer, metabolic disorders (such as diabetes), inflammatory and autoimmune disorders (such as inflammatory bowel diseases, e.g. Crohn's disease and ulcerative colitis, inflammatory pulmonary diseases, rheumatoid arthritis, lupus nephritis, systemic lupus erythematosus and psoriasis and psoriasis arthritis), neurological disorders (such as Alzheimer's disease, Parkinson's disease, multiple sclerosis, Charcot-Marie-Tooth neuropathy, amyotrophic lateral sclerosis and epilepsy), atherosclerosis and cardiovascular diseases, Sjogren Syndrome, renal allograft rejection, viral induced diseases, circulatory diseases, bone osteolysis and osteoporosis, osteoarthritis, sarcopenia, Langerhans cell histiocytosis, spinal cord injury, endometriosis, asthma and allergic asthma, eye diseases (such as retinopathies, age-related macular degeneration and uveitis) chronic and neuropathic pain, and fibro-proliferative diseases. Many of the compounds of this invention may show a favourable pharmacokinetic profile and have attractive properties in terms of bioavailability, including an acceptable half-life, AUC (area under the curve) and peak values and lacking unfavourable phenomena such as insufficient quick onset and tissue retention.

The combinations of the present invention may be used as medicaments. Said use as a medicine or method of treatment comprises the systemic administration to ill subjects of an amount effective to combat the conditions associated with the illnesses. Consequently, the combinations of the present invention can be used in the manufacture of a medicament useful for treating, preventing or combating illness or disease associated with protein kinases including cancer, metabolic disorders (such as diabetes), inflammatory and autoimmune disorders (such as inflammatory bowel diseases, e.g. Crohn's disease and ulcerative colitis, inflammatory pulmonary diseases, rheumatoid arthritis, lupus nephritis, systemic lupus erythematosus and psoriasis and psoriasis arthritis), neurological disorders (such as Alzheimer's disease, Parkinson's disease, multiple sclerosis, Charcot-Marie-Tooth neuropathy, amyotrophic lateral sclerosis and epilepsy), atherosclerosis and cardiovascular diseases, Sjogren Syndrome, renal allograft rejection, viral induced diseases, circulatory diseases, bone osteolysis and osteoporosis, osteoarthritis, sarcopenia, Langerhans cell histiocytosis, spinal cord injury, endometriosis, asthma and allergic asthma, eye diseases (such as retinopathies, age-related macular degeneration and uveitis) chronic and neuropathic pain, and fibro-proliferative diseases.

The term “therapeutically effective amount” as used herein means that amount of active compound or component or pharmaceutical agent that elicits the biological or medicinal response in a tissue, system, animal or human that is being sought, in the light of the present invention, by a researcher, veterinarian, medical doctor or other clinician, which includes alleviation of the symptoms of the disease being treated.

### EXAMPLES

### Example 1: General Procedure for the Preparation of Analogues 49-55

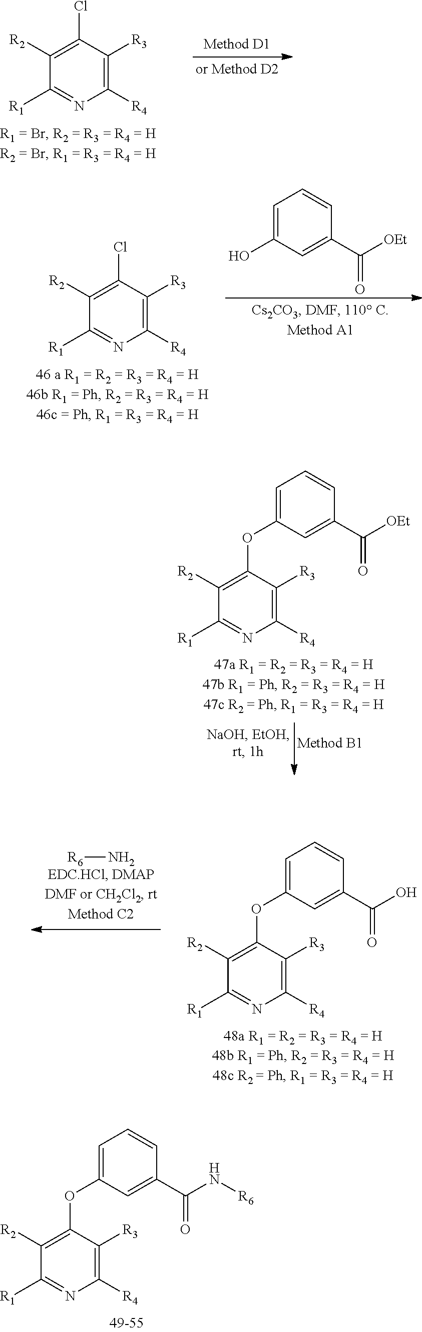

Method A1: To a solution of phenol derivative (1 equiv.) in DMF (5 mL/mmol) under nitrogen was added solid cesium carbonate (2.5 equiv.) followed by 4-chloropyridine derivative (1 equiv.). The reaction mixture was stirred at 110° C. until completion (from 2 h to overnight). After cooling at room temperature, a saturated aqueous solution of NH₄Cl was added and the aqueous layer was extracted with EtOAc. The organic layer was dried over Na₂SO₄, filtered and concentrated under reduced pressure. The crude was purified by flash column chromatography (cyclohexane/EtOAc from 100/0 to 50/50) or (DCM/MeOH from 100/0 to 90/10), or reverse phase chromatography (H₂O/MeOH: 0 to 100%) to give the expected compound.
Method B1: To a solution of appropriate intermediate 47 (1 equiv.) in EtOH or MeOH (2.5 mL/mmol) was added a solution of NaOH 1N (2.9 mL/mmol). The reaction mixture was stirred at rt until completion. EtOH or MeOH was removed under reduce pressure and the crude was acidified with HCl 1N until pH=2-3. The precipitate was filtered-off, washed with water and dried over P₂O₅ in vacuum to give the expected intermediate 48.
Method C2: To a suspension of appropriate intermediate 48 (1 equiv.) in DCM or DMF (10 mL/mmol) under nitrogen were added DMAP (2.2 equiv.), EDC·HCl (2 equiv.) and appropriate amine (1.1-1.5 equiv.). The reaction mixture was stirred at room temperature until completion (1 h-overnight). The reaction mixture was diluted with DCM and washed twice with a saturated solution of NH₄Cl. The organic layer was dried over Na₂SO₄, filtered and concentrated under reduced pressure. The crude product was purified by flash column chromatography (DCM/MeOH from 100/0 to 90/10) and reverse phase chromatography (H₂O/MeOH: 0 to 100%) to give the expected compound.
Method D1: To a stirred solution of 2-bromo-4-chloropyridine (192 mg, 1 mmol) in isopropanol/H₂O (4 mL/4 mL) were added phenylboronic acid (128 mg, 1.05 mmol), K₃PO₄ (424 mg, 2 mmol) and Pd(OAc)₂ (4 mg, 0.015 mmol). The reaction mixture was stirred at 80° C. under air atmosphere for 30 minutes. After cooling at room temperature, the reaction mixture was diluted with EtOAc and washed twice with a saturated solution of NaCl. The organic layer was dried over Na₂SO₄, filtered and concentrated under reduced pressure. The crude was purified by flash column chromatography (Cyclohexane/EtOAc: 100/0 to 90/10) to give 162 mg of 4-chloro-2-phenylpyridine 46b in 85% yield.
Method D2: To a stirred solution of 3-bromo-4-chloropyridine (385 mg, 2 mmol) in dioxane (10 mL) were added under nitrogen phenylboronic acid (256 mg, 2.1 mmol), solid K₃PO₄ (849 mg, 4 mmol) and Pd(PPh₃)₄ (231 mg, 0.2 mmol). The reaction mixture was stirred at 100° C. for 4 h. The solvent was removed under vacuum, and the crude was purified by flash column chromatography (cyclohexane/EtOAc from 100/0 to 75/25), to give 4-chloro-3-phenyl-pyridine 46c as a yellow oil in 87% yield.

The following table illustrates intermediates 47 prepared from Method A1:

| Intermediate | Structure | Synthesis procedure |
|---|---|---|
| Compound 47a | 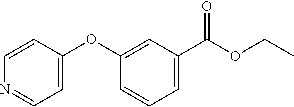 [SMILES: CCOC(=O)c1cccc(Oc2ccncc2)c1] | Method A1 |
| Compound 47b | 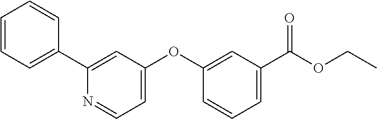 [SMILES: CCOC(=O)c1cccc(Oc2ccnc(-c3ccccc3)c2)c1] | Method A1 |
| Compound 47c | 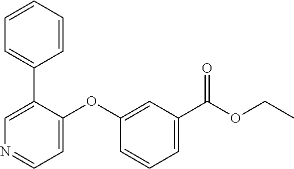 [SMILES: CCOC(=O)c1cccc(Oc2ccncc2-c2ccccc2)c1] | Method A1 |

The following table illustrates intermediates 48 prepared from Method B1:

| Intermediate | Structure | Synthesis procedure |
|---|---|---|
| Compound 48a | 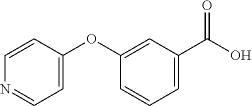 [SMILES: O=C(O)c1cccc(Oc2ccncc2)c1] | Method B1 |
| Compound 48b | 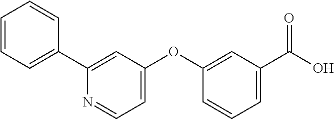 [SMILES: O=C(O)c1cccc(Oc2ccnc(-c3ccccc3)c2)c1] | Method B1 |
| Compound 48c | 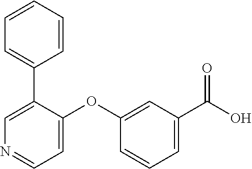 [SMILES: O=C(O)c1cccc(Oc2ccncc2-c2ccccc2)c1] | Method B1 |

The following compounds are examples illustrating procedure C2:

### N-(cyclohexylmethyl)-3-(4-pyridyloxy)benzamide (49)

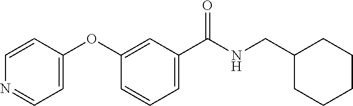 [SMILES: O=C(NCC1CCCCC1)c1cccc(Oc2ccncc2)c1]

Compound 49 was synthesized from intermediate 48a (0.20 mmol) and 1-cyclohexylmethanamine (0.34 mmol) as a white solid in 83% yield according to the general method C2. ¹H NMR (600 MHz, DMSO-d₆) δ (ppm): 8.51 (t, J=5.8 Hz, 1H), 8.49-8.47 (m, 2H), 7.78 (ddd, J=7.8 Hz, 1.5 Hz, 1.0 Hz, 1H), 7.64-7.61 (m, 1H), 7.57 (t, J=7.9 Hz, 1H), 7.35 (ddd, J=8.1 Hz, 2.5 Hz, 1.0 Hz, 1H), 6.96-6.93 (m, 2H), 3.09 (dd, J=6.8 Hz, 6.0 Hz, 2H), 1.68 (t, J=13.5 Hz, 4H), 1.63-1.49 (m, 2H), 1.23-1.09 (m, 3H), 0.95-0.84 (m, 2H).

### N-(cyclohexylmethyl)-3-[(2-phenyl-4-pyridyl)oxy]benzamide (50)

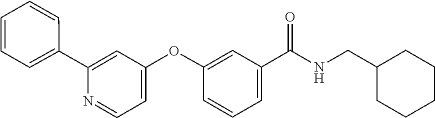 [SMILES: O=C(NCC1CCCCC1)c1cccc(Oc2ccnc(-c3ccccc3)c2)c1]

Compound 50 was synthesized from intermediate 48b (0.20 mmol) and 1-cyclohexylmethanamine (0.30 mmol) as a white solid in 70% yield according to the general method C2. ¹H NMR (400 MHz, CDCl₃) δ (ppm): 8.56 (d, J=5.6 Hz, 1H), 7.94-7.88 (m, 2H), 7.66-7.61 (m, 1H), 7.57-7.54 (m, 1H), 7.52-7.39 (m, 4H), 7.28-7.24 (m, 2H), 6.79 (dd, J=5.6 Hz, 2.4 Hz, 1H), 6.14 (bs, 1H), 3.35-3.28 (m, 2H), 1.83-1.54 (m, 6H), 1.27-1.15 (m, 3H), 1.05-0.94 (m, 2H).

### Example 2: General Procedure for the Synthesis of Analogues 68-101

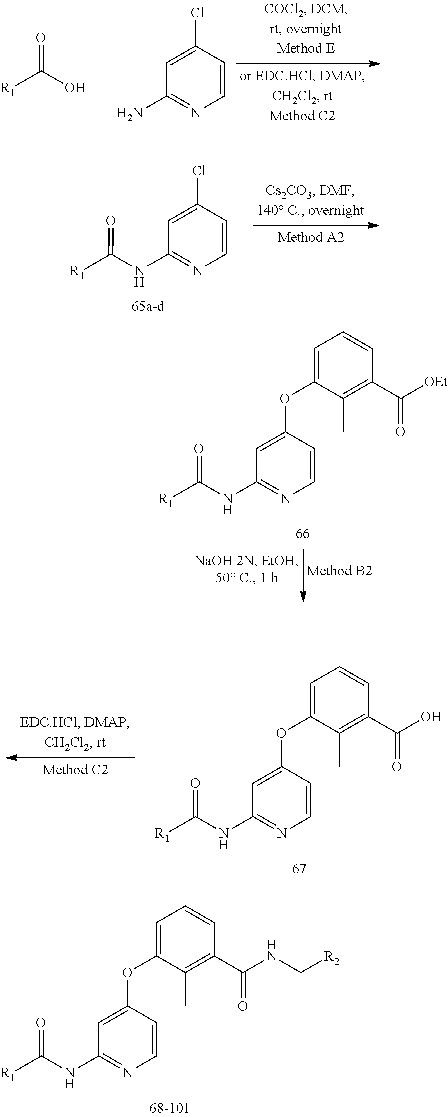

Method E: To a solution of carboxylic acid derivative (1 equiv.) in CH₂Cl₂ (5 mL/mmol) under nitrogen were added oxalyl chloride (3 equiv.) and 50 μL of DMF. The reaction mixture was stirred at room temperature overnight. The reaction mixture was concentrated under reduced pressure to give the acyl chloride derivative. To a solution of this previous intermediate in pyridine (3 mL/mmol) under nitrogen was added 2-amino-4-chloropyridine (1 equiv.) and the reaction mixture was stirred at room temperature until completion (from 2 h to overnight). The reaction mixture was concentrated under reduced pressure. The crude was purified by flash column chromatography (DCM/MeOH from 100/0 to 95/5) to give the expected compound.

The following compound 65a is an example illustrating Method E:

### Preparation of N-(4-chloro-2-pyridyl)-1-methyl-pyrazole-4-carboxamide (65a)

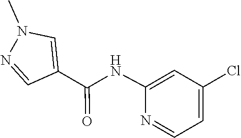 [SMILES: Cn1cc(C(=O)Nc2cc(Cl)ccn2)cn1]

Intermediate 65a was synthesized from 1-methyl-1H-pyrazole-4-carboxylic acid (2.37 mmol) and 2-amino-4-chloropyridine (2.37 mmol) as a white powder in 84% yield according to the general method E.

ESI-MS: 237.10 (M+H)⁺.

The following table illustrates intermediates 65 prepared from method E:

| Intermediate | Structure | Synthesis procedure |
|---|---|---|
| Compound 65a | 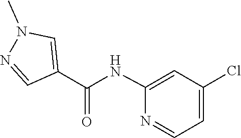 [SMILES: Cn1cc(C(=O)Nc2cc(Cl)ccn2)cn1] | Method E |
| Compound 65b | 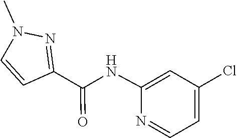 [SMILES: Cn1ccc(C(=O)Nc2cc(Cl)ccn2)n1] | Method E |
| Compound 65c | 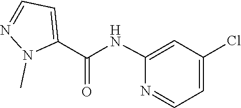 [SMILES: Cn1nccc1C(=O)Nc1cc(Cl)ccn1] | Method E |
| Compound 65d | 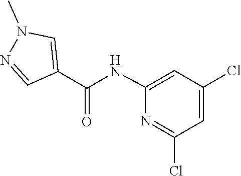 [SMILES: Cn1cc(C(=O)Nc2cc(Cl)cc(Cl)n2)cn1] | Method E |

The following compound 65e is an example illustrating Method C2:

### Preparation of N-(4-chloro-2-pyridyl)pyridine-3-carboxamide (65e)

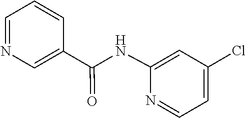 [SMILES: O=C(Nc1cc(Cl)ccn1)c1cccnc1]

Intermediate 65e was synthesized from nicotinic acid (1.28 mmol) and 2-amino-4-chloropyridine (1.16 mmol) as a white powder in 84% yield according to the general method C2.

ESI-MS: 234.10 (M+H)⁺.
Method A2: To a solution of phenol derivative (1 equiv.) in DMF (2 mL/mmol) under nitrogen was added solid cesium carbonate (2.5 equiv.) followed by 4-chloropyridine derivative (1 equiv.). The reaction mixture was stirred at 140° C. overnight. The reaction mixture was concentrated under reduced pressure to give the expected compound which was used in the next step without purification.

The following compound 66a is an example illustrating Method A2: Preparation of ethyl 3-({2-[(1-methylpyrazole-4-carbonyl)amino]-4-pyridyl}oxy)benzoate (66a)

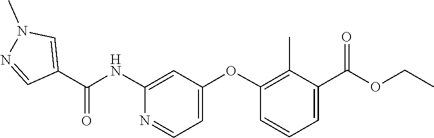 [SMILES: CCOC(=O)c1cccc(Oc2ccnc(NC(=O)c3cnn(C)c3)c2)c1C]

Intermediate 66a was synthesized from ethyl 3-hydroxy-2-methyl-benzoate (0.91 mmol) and compound 65a (0.91 mmol) as a brown powder according to the general method A2.

ESI-MS: 381.20 (M+H)⁺.

The following table illustrates intermediates 66 prepared from method A2:

| Intermediate | Structure | Synthesis procedure |
|---|---|---|
| Compound 66a | 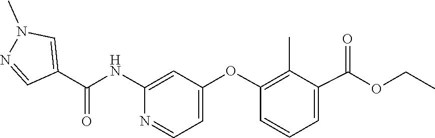 [SMILES: CCOC(=O)c1cccc(Oc2ccnc(NC(=O)c3cnn(C)c3)c2)c1C] | Method A2 |
| Compound 66b | 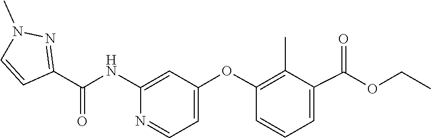 [SMILES: CCOC(=O)c1cccc(Oc2ccnc(NC(=O)c3ccn(C)n3)c2)c1C] | Method A2 |
| Compound 66c | 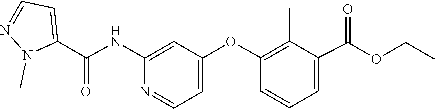 [SMILES: CCOC(=O)c1cccc(Oc2ccnc(NC(=O)c3ccnn3C)c2)c1C] | Method A2 |
| Compound 66d | 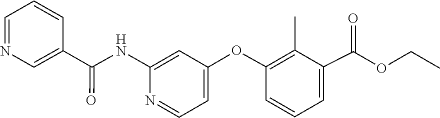 [SMILES: CCOC(=O)c1cccc(Oc2ccnc(NC(=O)c3cccnc3)c2)c1C] | Method A2 |

Method B2: To a solution of appropriate intermediate 66 (1 equiv.) in EtOH (2.5 mL/mmol) was added a solution of NaOH 2N (2.5 mL/mmol). The reaction mixture was stirred at 50° C. for 1 h. EtOH was evaporated under reduced pressure and the residue was dissolved in water and washed 3 times with CH₂Cl₂. The aqueous layer was then acidified with concentrated HCl until pH=2-3. The resulting precipitate was filtered, washed with H₂O and dried over P₂O₅ to afford the expected compound. If necessary, the filtrate was evaporated under reduced pressure and purified by reverse phase chromatography (H₂O/MeOH from 100/0 to 0/100) to give more expected compound.

The following compound 67a is an example illustrating Method B2:

### Preparation of 2-methyl-3-({2-[(1-methylpyrazole-4-carbonyl)amino]-4-pyridyl}oxy)benzoic acid (67a)

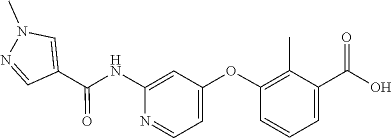 [SMILES: Cc1c(Oc2ccnc(NC(=O)c3cnn(C)c3)c2)cccc1C(=O)O]

Intermediate 67a was synthesized from intermediate 66a (0.91 mmol) as a white solid in 32% yield (over 2 steps) according to the general method B2.

ESI-MS: 353.15 (M+H)⁺.

The following table illustrates intermediates 67 prepared from method B2:

| Intermediate | Structure | Synthesis procedure |
|---|---|---|
| Compound 67a | 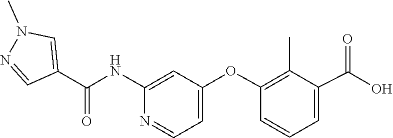 [SMILES: Cc1c(Oc2ccnc(NC(=O)c3cnn(C)c3)c2)cccc1C(=O)O] | Method B2 |
| Compound 67b | 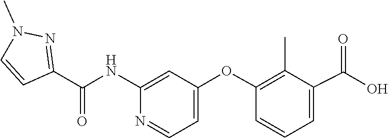 [SMILES: Cc1c(Oc2ccnc(NC(=O)c3ccn(C)n3)c2)cccc1C(=O)O] | Method B2 |
| Compound 67c | 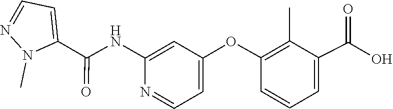 [SMILES: Cc1c(Oc2ccnc(NC(=O)c3ccnn3C)c2)cccc1C(=O)O] | Method B2 |
| Compound 67d | 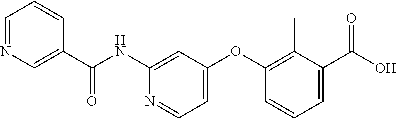 [SMILES: Cc1c(Oc2ccnc(NC(=O)c3cccnc3)c2)cccc1C(=O)O] | Method B2 |

The following compounds are examples illustrating Method C2:

### N-(4-{3-[(2,6-difluoro-4-pyridyl)methylcarbamoyl]-2-methyl-phenoxy}-2-pyridyl)-1-methyl-pyrazole-4-carboxamide (68)

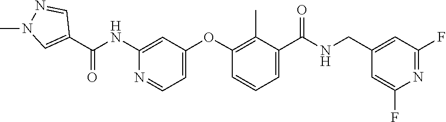 [SMILES: Cc1c(Oc2ccnc(NC(=O)c3cnn(C)c3)c2)cccc1C(=O)NCc1cc(F)nc(F)c1]

Compound 68 was synthesized from intermediate 67a (0.42 mmol) and (2,6-difluoro-4-pyridyl) methanamine (0.63 mmol) as a white solid in 23% yield according to the general method C2. ¹H NMR (400 MHz, DMSO-d₆) δ (ppm): 10.59 (s, 1H), 9.16 (t, J=6.0 Hz, 1H), 8.40 (s, 1H), 8.24 (d, J=5.7 Hz, 1H), 8.09 (s, 1H), 7.75 (d, J=2.3 Hz, 1H), 7.50-7.37 (m, 2H), 7.30-7.24 (m, 1H), 7.14 (s, 2H), 6.63 (dd, J=5.7 Hz, 2.4 Hz, 1H), 4.57 (d, J=5.9 Hz, 2H), 3.86 (s, 3H), 2.15 (s, 3H).

ESI-MS: 479.20 (M+H)⁺.

### N-(4-{3-[(4-cyano-3-fluoro-phenyl)methylcarbamoyl]-2-methyl-phenoxy}-2-pyridyl)-1-methyl-pyrazole-4-carboxamide (69)

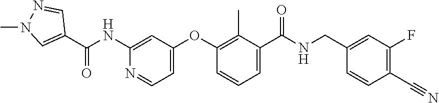 [SMILES: Cc1c(Oc2ccnc(NC(=O)c3cnn(C)c3)c2)cccc1C(=O)NCc1ccc(C#N)c(F)c1]

Compound 69 was synthesized from intermediate 67a (0.10 mmol) and 4-aminomethyl-2-fluorobenzonitrile (0.11 mmol) as a white solid in 71% yield according to the general method C2. ¹H NMR (400 MHz, DMSO-d₆) δ (ppm): 10.59 (s, 1H), 9.14 (t, J=6.0 Hz, 1H), 8.40 (s, 1H), 8.23 (d, J=5.7 Hz, 1H), 8.09 (d, J=0.6 Hz, 1H), 7.97-7.88 (m, 1H), 7.74 (d, J=2.3 Hz, 1H), 7.48 (d, J=10.4 Hz, 1H), 7.43-7.37 (m, 3H), 7.26 (t, J=4.7 Hz, 1H), 6.63 (dd, J=5.7 Hz, 2.4 Hz, 1H), 4.54 (d, J=5.9 Hz, 2H), 3.86 (s, 3H), 2.14 (s, 3H).

ESI-MS: 485.15 (M+H)⁺.

### N-(4-{3-[(3,5-difluorophenyl)methylcarbamoyl]-2-methyl-phenoxy}-2-pyridyl)-1-methyl-pyrazole-4-carboxamide (70)

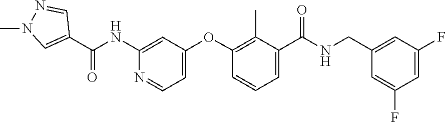 [SMILES: Cc1c(Oc2ccnc(NC(=O)c3cnn(C)c3)c2)cccc1C(=O)NCc1cc(F)cc(F)c1]

Compound 70 was synthesized from intermediate 67a (0.10 mmol) and 3,5-difluorobenzylamine (0.11 mmol) as a white solid in 64% yield according to the general method C2. ¹H NMR (400 MHz, DMSO-d₆) δ (ppm): 10.56 (s, 1H), 9.05 (t, J=6.1 Hz, 1H), 8.40 (s, 1H), 8.23 (d, J=5.7 Hz, 1H), 8.09 (s, 1H), 7.75 (d, J=2.3 Hz, 1H), 7.44-7.35 (m, 2H), 7.25 (dd, J=7.3 Hz, 2.0 Hz, 1H), 7.18-7.03 (m, 3H), 6.62 (dd, J=5.7 Hz, 2.4 Hz, 1H), 4.47 (d, J=6.0 Hz, 2H), 3.86 (s, 3H), 2.14 (s, 3H).

### N-(4-{3-[(6-methoxy-3-pyridyl)methylcarbamoyl]-2-methyl-phenoxy}-2-pyridyl)-1-methyl-pyrazole-4-carboxamide (71)

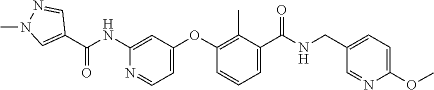

Compound 71 was synthesized from intermediate 67a (0.10 mmol) and (6-methoxypyridin-3-yl) methanamine (0.11 mmol) as a white solid in 61% yield according to the general method C2. ¹H NMR (400 MHz, DMSO-d₆) δ (ppm): 10.56 (s, 1H), 8.95 (t, J=5.9 Hz, 1H), 8.40 (s, 1H), 8.22 (d, J=5.7 Hz, 1H), 8.14 (d, J=2.2 Hz, 1H), 8.09 (s, 1H), 7.74 (d, J=2.3 Hz, 1H), 7.69 (dd, J=8.5 Hz, 2.5 Hz, 1H), 7.40-7.19 (m, 3H), 6.81 (d, J=8.5 Hz, 1H), 6.61 (dd, J=5.7 Hz, 2.4 Hz, 1H), 4.38 (d, J=5.9 Hz, 2H), 3.86 (s, 3H), 3.83 (s, 3H), 2.11 (s, 3H).

### N-(4-{3-[(3-fluorophenyl)methylcarbamoyl]-2-methyl-phenoxy}-2-pyridyl)-1-methyl-pyrazole-4-carboxamide (72)

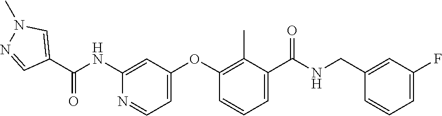 [SMILES: Cc1c(Oc2ccnc(NC(=O)c3cnn(C)c3)c2)cccc1C(=O)NCc1cccc(F)c1]

Compound 72 was synthesized from intermediate 67a (0.09 mmol) and 3-fluorobenzylamine (0.13 mmol) as a white solid in 26% yield according to the general method C2. ¹H NMR (400 MHz, DMSO-d₆) δ (ppm): 10.54 (s, 1H), 9.01 (t, J=6.1 Hz, 1H), 8.40 (s, 1H), 8.23 (d, J=5.8 Hz, 1H), 8.09 (d, J=0.6 Hz, 1H), 7.75 (d, J=2.3 Hz, 1H), 7.45-7.20 (m, 6H), 7.24 (dd, J=7.9 Hz, 1.3 Hz, 1H), 6.62 (dd, J=5.7 Hz, 2.4 Hz, 1H), 4.46 (d, J=6.0 Hz, 2H), 3.86 (s, 3H), 2.14 (s, 3H).

ESI-MS: 460.15 (M+H)⁺.

### N-(4-{3-[(4-fluorophenyl)methylcarbamoyl]-2-methyl-phenoxy}-2-pyridyl)-1-methyl-pyrazole-4-carboxamide (73)

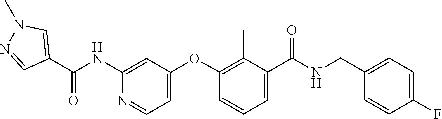 [SMILES: Cc1c(Oc2ccnc(NC(=O)c3cnn(C)c3)c2)cccc1C(=O)NCc1ccc(F)cc1]

Compound 73 was synthesized from intermediate 67a (0.09 mmol) and 4-fluorobenzylamine (0.13 mmol) as a white solid in 26% yield according to the general method C2. ¹H NMR (400 MHz, DMSO-d₆) δ (ppm): 10.53 (s, 1H), 9.00 (t, J=6.0 Hz, 1H), 8.40 (s, 1H), 8.23 (d, J=5.7 Hz, 1H), 8.09 (d, J=0.5 Hz, 1H), 7.75 (d, J=2.3 Hz, 1H), 7.46-7.32 (m, 3H), 7.26-7.04 (m, 4H), 6.62 (dd, J=5.7 Hz, 2.4 Hz, 1H), 4.47 (d, J=6.0 Hz, 2H), 3.86 (s, 3H), 2.14 (s, 3H).

ESI-MS: 460.15 (M+H)⁺.

### N-(4-{3-[(3-chlorophenyl)methylcarbamoyl]-2-methyl-phenoxy}-2-pyridyl)-1-methyl-pyrazole-4-carboxamide (74)

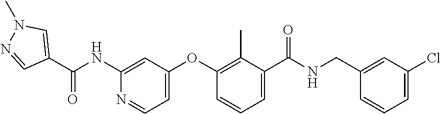 [SMILES: Cc1c(Oc2ccnc(NC(=O)c3cnn(C)c3)c2)cccc1C(=O)NCc1cccc(Cl)c1]

Compound 74 was synthesized from intermediate 67a (0.09 mmol) and 3-chlorobenzylamine (0.13 mmol) as a white solid in 30% yield according to the general method C2. ¹H NMR (400 MHz, DMSO-d₆) δ (ppm): 10.53 (s, 1H), 8.97 (t, J=6.2 Hz, 1H), 8.40 (s, 1H), 8.23 (d, J=5.7 Hz, 1H), 8.09 (d, J=0.5 Hz, 1H), 7.75 (d, J=2.3 Hz, 1H), 7.47-7.30 (m, 4H), 7.25-7.12 (m, 3H), 6.61 (dd, J=5.7 Hz, 2.4 Hz, 1H), 4.43 (d, J=5.9 Hz, 2H), 3.86 (s, 3H), 2.13 (s, 3H).

ESI-MS: 476.10 (M+H)⁺.

### N-(4-{3-[(4-chlorophenyl)methylcarbamoyl]-2-methyl-phenoxy}-2-pyridyl)-1-methyl-pyrazole-4-carboxamide (75)

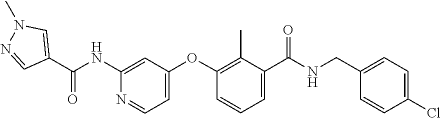 [SMILES: Cc1c(Oc2ccnc(NC(=O)c3cnn(C)c3)c2)cccc1C(=O)NCc1ccc(Cl)cc1]

Compound 75 was synthesized from intermediate 67a (0.09 mmol) and 4-chlorobenzylamine (0.13 mmol) as a white solid in 30% yield according to the general method C2. ¹H NMR (400 MHz, DMSO-d₆) δ (ppm): 10.56 (s, 1H), 9.01 (t, J=6.0 Hz, 1H), 8.40 (s, 1H), 8.23 (d, J=5.7 Hz, 1H), 8.09 (d, J=0.4 Hz, 1H), 7.75 (d, J=2.3 Hz, 1H), 7.47-7.31 (m, 6H), 7.23 (dd, J=7.8 Hz, 1.3 Hz, 1H), 6.62 (dd, J=5.7 Hz, 2.4 Hz, 1H), 4.43 (d, J=6.0 Hz, 2H), 3.86 (s, 3H), 2.13 (s, 3H).

ESI-MS: 476.10 (M+H)⁺.

### N-{4-[3-(imidazo[1,2-a]pyridin-6-ylmethylcarbamoyl)-2-methyl-phenoxy]-2-pyridyl}-1-methyl-pyrazole-4-carboxamide (76)

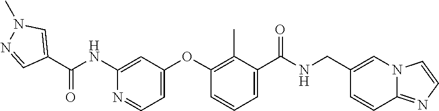 [SMILES: Cc1c(Oc2ccnc(NC(=O)c3cnn(C)c3)c2)cccc1C(=O)NCc1ccc2nccn2c1]

Compound 76 was synthesized from intermediate 67a (0.09 mmol) and imidazo[1,2-a]pyridin-6-ylmethanamine (0.13 mmol) as a white solid in 22% yield according to the general method C2. ¹H NMR (400 MHz, DMSO-d₆) δ (ppm): 10.56 (s, 1H), 9.01 (t, J=5.9 Hz, 1H), 8.50 (s, 1H), 8.40 (s, 1H), 8.23 (d, J=5.7 Hz, 1H), 8.09 (d, J=0.5 Hz, 1H), 7.97 (s, 1H), 7.74 (d, J=2.3 Hz, 1H), 7.58-7.52 (m, 2H), 7.45-7.32 (m, 2H), 7.29-7.19 (m, 2H), 6.62 (dd, J=5.7 Hz, 2.4 Hz, 1H), 4.44 (d, J=5.9 Hz, 2H), 3.86 (s, 3H), 2.13 (s, 3H).

### N-{4-[3-(imidazo[1,2-a]pyridin-7-ylmethylcarbamoyl)-2-methyl-phenoxy]-2-pyridyl}-1-methyl-pyrazole-4-carboxamide (77)

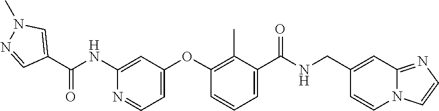 [SMILES: Cc1c(Oc2ccnc(NC(=O)c3cnn(C)c3)c2)cccc1C(=O)NCc1ccn2ccnc2c1]

Compound 77 was synthesized from intermediate 67a (0.09 mmol) and imidazo[1,2-a]pyridin-7-ylmethanamine (0.13 mmol) as a white solid in 15% yield according to the general method C2. ¹H NMR (400 MHz, DMSO-d₆) δ (ppm): 10.56 (s, 1H), 9.05 (t, J=6.1 Hz, 1H), 8.51 (dd, J=7.0 Hz, 0.7 Hz, 1H), 8.40 (s, 1H), 8.23 (d, J=5.7 Hz, 1H), 8.09 (d, J=0.5 Hz, 1H), 7.90 (s, 1H), 7.75 (d, J=2.3 Hz, 1H), 7.53 (d, J=1.2 Hz, 1H), 7.47-7.35 (m, 3H), 7.25 (dd, J=7.4 Hz, 1.9 Hz, 1H), 6.89 (dd, J=7.0 Hz, 1.6 Hz, 1H), 6.62 (dd, J=5.7 Hz, 2.4 Hz, 1H), 4.49 (d, J=5.9 Hz, 2H), 3.86 (s, 3H), 2.15 (s, 3H).

ESI-MS: 482.2 0 (M+H)⁺.

### 1-methyl-N-[4-(2-methyl-3-{[6-(trifluoromethyl)-3-pyridyl]methylcarbamoyl}phenoxy)-2-pyridyl]pyrazole-4-carboxamide (78)

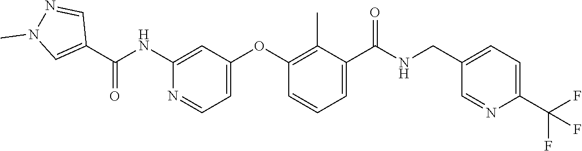 [SMILES: Cc1c(Oc2ccnc(NC(=O)c3cnn(C)c3)c2)cccc1C(=O)NCc1ccc(C(F)(F)F)nc1]

Compound 78 was synthesized from intermediate 67a (0.09 mmol) and [6-(trifluoromethyl)-3-pyridyl]methanamine (0.13 mmol) as a white solid in 44% yield according to the general method C2. ¹H NMR (400 MHz, DMSO-d₆) δ (ppm): 10.88 (s, 1H), 9.12 (t, J=5.9 Hz, 1H), 8.76 (s, 1H), 8.27 (d, J=5.7 Hz, 1H), 8.05 (d, J=9.3 Hz, 1H), 7.91 (d, J=8.1 Hz, 1H), 7.71 (d, J=2.3 Hz, 1H), 7.50 (d, J=2.1 Hz, 1H), 7.46-7.35 (m, 2H), 7.30-7.23 (m, 2H), 6.65 (dd, J=5.7 Hz, 2.3 Hz, 1H), 4.58 (d, J=5.8 Hz, 2H), 4.04 (s, 3H), 2.14 (s, 3H).

ESI-MS: 511.15 (M+H)⁺.

### N-(4-{3-[(5-fluoro-6-methoxy-3-pyridyl)methylcarbamoyl]-2-methyl-phenoxy}-2-pyridyl)-1-methyl-pyrazole-4-carboxamide (79)

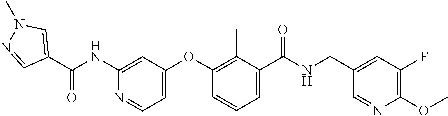 [SMILES: COc1ncc(CNC(=O)c2cccc(Oc3ccnc(NC(=O)c4cnn(C)c4)c3)c2C)cc1F]

Compound 79 was synthesized from intermediate 67a (0.09 mmol) and (5-fluoro-6-methoxy-3-pyridyl) methanamine (0.13 mmol) as a white solid in 38% yield according to the general method C2. ¹H NMR (400 MHz, DMSO-d₆) δ (ppm): 10.54 (s, 1H), 8.95 (t, J=5.9 Hz, 1H), 8.40 (s, 1H), 8.23 (d, J=5.7 Hz, 1H), 8.09 (d, J=0.4 Hz, 1H), 7.98 (d, J=1.8 Hz, 1H), 7.74 (d, J=2.3 Hz, 1H), 7.65 (dd, J=11.4 Hz, 1.9 Hz, 1H), 7.41-7.30 (m, 2H), 7.23 (dd, J=7.8 Hz, 1.4 Hz, 1H), 6.61 (dd, J=5.7 Hz, 2.4 Hz, 1H), 4.41 (d, J=5.9 Hz, 2H), 3.93 (s, 3H), 3.86 (s, 3H), 2.12 (s, 3H).

ESI-MS: 491.05 (M+H)⁺.

### N-(4-{3-[(3-fluorophenyl)methylcarbamoyl]-2-methyl-phenoxy}-2-pyridyl)-1-methyl-pyrazole-3-carboxamide (80)

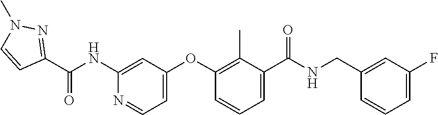 [SMILES: Cc1c(Oc2ccnc(NC(=O)c3ccn(C)n3)c2)cccc1C(=O)NCc1cccc(F)c1]

Compound 80 was synthesized from intermediate 67b (0.11 mmol) and 3-fluorobenzylamine (0.17 mmol) as a white solid in 37% yield according to the general method C2. ¹H NMR (400 MHz, DMSO-d₆) δ (ppm): 9.54 (s, 1H), 9.03 (t, J=6.0 Hz, 1H), 8.23 (d, J=5.8 Hz, 1H), 7.87 (d, J=2.3 Hz, 1H), 7.71 (d, J=2.3 Hz, 1H), 7.45-7.34 (m, 3H), 7.29-7.14 (m, 3H), 7.09 (td, J=8.5 Hz, 2.5 Hz, 1H), 6.81 (d, J=2.3 Hz, 1H), 6.65 (dd, J=5.7 Hz, 2.4 Hz, 1H), 4.47 (d, J=6.0 Hz, 2H), 3.95 (s, 3H), 2.14 (s, 3H).

ESI-MS: 460.30 (M+H)⁺.

### N-(4-{3-[(4-fluorophenyl)methylcarbamoyl]-2-methyl-phenoxy}-2-pyridyl)-1-methyl-pyrazole-3-carboxamide (81)

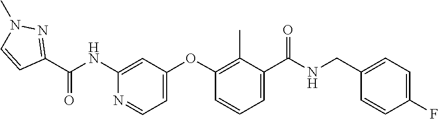 [SMILES: Cc1c(Oc2ccnc(NC(=O)c3ccn(C)n3)c2)cccc1C(=O)NCc1ccc(F)cc1]

Compound 81 was synthesized from intermediate 67b (0.11 mmol) and 4-fluorobenzylamine (0.17 mmol) as a white solid in 37% yield according to the general method C2. ¹H NMR (400 MHz, DMSO-d₆) δ (ppm): 9.54 (s, 1H), 8.99 (t, J=6.1 Hz, 1H), 8.23 (d, J=5.7 Hz, 1H), 7.88 (d, J=2.3 Hz, 1H), 7.71 (d, J=2.3 Hz, 1H), 7.42-7.33 (m, 4H), 7.25 (dd, J=7.8 Hz, 1.4 Hz, 1H), 7.22-7.12 (m, 2H), 6.81 (d, J=2.3 Hz, 1H), 6.65 (dd, J=5.7 Hz, 2.4 Hz, 1H), 4.43 (d, J=6.0 Hz, 2H), 3.95 (s, 3H), 2.13 (s, 3H).

ESI-MS: 460.25 (M+H)⁺.

### N-(4-{3-[(3-chlorophenyl)methylcarbamoyl]-2-methyl-phenoxy}-2-pyridyl)-1-methyl-pyrazole-3-carboxamide (82)

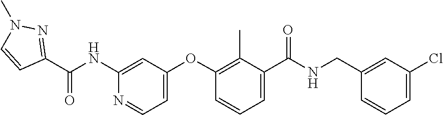 [SMILES: Cc1c(Oc2ccnc(NC(=O)c3ccn(C)n3)c2)cccc1C(=O)NCc1cccc(Cl)c1]

Compound 82 was synthesized from intermediate 67b (0.11 mmol) and 3-chlorobenzylamine (0.17 mmol) as a white solid in 41% yield according to the general method C2. ¹H NMR (400 MHz, DMSO-d₆) δ (ppm): 9.54 (s, 1H), 9.03 (t, J=6.1 Hz, 1H), 8.23 (d, J=5.7 Hz, 1H), 7.87 (d, J=2.3 Hz, 1H), 7.71 (d, J=2.3 Hz, 1H), 7.45-7.30 (m, 6H), 7.26 (dd, J=7.8 Hz, 1.2 Hz, 1H), 6.81 (d, J=2.3 Hz, 1H), 6.65 (dd, J=5.7 Hz, 2.4 Hz, 1H), 4.46 (d, J=6.0 Hz, 2H), 3.95 (s, 3H), 2.14 (s, 3H).

ESI-MS: 476.10 (M+H)⁺.

### N-(4-{3-[(4-chlorophenyl)methylcarbamoyl]-2-methyl-phenoxy}-2-pyridyl)-1-methyl-pyrazole-3-carboxamide (83)

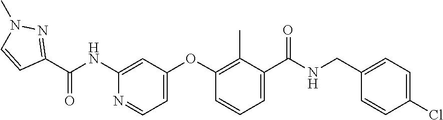 [SMILES: Cc1c(Oc2ccnc(NC(=O)c3ccn(C)n3)c2)cccc1C(=O)NCc1ccc(Cl)cc1]

Compound 83 was synthesized from intermediate 67b (0.11 mmol) and 4-chlorobenzylamine (0.17 mmol) as a white solid in 48% yield according to the general method C2. ¹H NMR (400 MHz, DMSO-d₆) δ (ppm): 9.52 (s, 1H), 8.99 (t, J=6.0 Hz, 1H), 8.23 (d, J=5.7 Hz, 1H), 7.87 (d, J=2.3 Hz, 1H), 7.71 (d, J=2.3 Hz, 1H), 7.44-7.32 (m, 6H), 7.25 (dd, J=7.8 Hz, 1.4 Hz, 1H), 6.80 (d, J=2.3 Hz, 1H), 6.65 (dd, J=5.7 Hz, 2.4 Hz, 1H), 4.44 (d, J=6.0 Hz, 2H), 3.95 (s, 3H), 2.13 (s, 3H).

ESI-MS: 476.10 (M+H)⁺.

### N-{4-[3-(imidazo[1,2-a]pyridin-6-ylmethylcarbamoyl)-2-methyl-phenoxy]-2-pyridyl}-1-methyl-pyrazole-3-carboxamide (84)

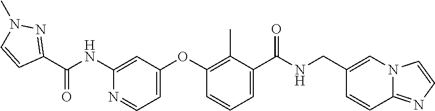 [SMILES: Cc1c(Oc2ccnc(NC(=O)c3ccn(C)n3)c2)cccc1C(=O)NCc1ccc2nccn2c1]

Compound 84 was synthesized from intermediate 67b (0.11 mmol) and imidazo[1,2-a]pyridin-6-ylmethanamine (0.17 mmol) as a white solid in 35% yield according to the general method C2. ¹H NMR (400 MHz, DMSO-d₆) δ (ppm): 9.52 (s, 1H), 8.99 (t, J=6.0 Hz, 1H), 8.50 (s, 1H), 8.22 (d, J=5.8 Hz, 1H), 7.96 (s, 1H), 7.87 (d, J=2.3 Hz, 1H), 7.70 (d, J=2.3 Hz, 1H), 7.59-7.52 (m, 2H), 7.44-7.34 (m, 2H), 7.28-7.22 (m, 2H), 6.80 (d, J=2.3 Hz, 1H), 6.65 (dd, J=5.7 Hz, 2.4 Hz, 1H), 4.45 (d, J=5.8 Hz, 2H), 3.95 (s, 3H), 2.14 (s, 3H).

### N-{4-[3-(imidazo[1,2-a]pyridin-7-ylmethylcarbamoyl)-2-methyl-phenoxy]-2-pyridyl}-1-methyl-pyrazole-3-carboxamide (85)

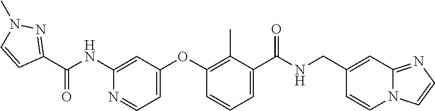 [SMILES: Cc1c(Oc2ccnc(NC(=O)c3ccn(C)n3)c2)cccc1C(=O)NCc1ccn2ccnc2c1]

Compound 85 was synthesized from intermediate 67b (0.11 mmol) and imidazo[1,2-a]pyridin-7-ylmethanamine (0.17 mmol) as a white solid in 22% yield according to the general method C2. ¹H NMR (400 MHz, DMSO-d₆) δ (ppm): 9.52 (s, 1H), 9.04 (t, J=6.0 Hz, 1H), 8.51 (dd, J=7.0 Hz, 0.7 Hz, 1H), 8.23 (d, J=5.8 Hz, 1H), 7.90 (s, 1H), 7.87 (d, J=2.3 Hz, 1H), 7.72 (d, J=2.3 Hz, 1H), 7.53 (d, J=1.1 Hz, 1H), 7.45-7.37 (m, 3H), 7.26 (dd, J=7.1 Hz, 2.2 Hz, 1H), 6.89 (dd, J=7.0 Hz, 1.6 Hz, 1H), 6.81 (d, J=2.3 Hz, 1H), 6.65 (dd, J=5.7 Hz, 2.4 Hz, 1H), 4.49 (d, J=5.9 Hz, 2H), 3.95 (s, 3H), 2.16 (s, 3H).

ESI-MS: 482.15 (M+H)⁺.

### N-(4-{3-[(3,5-difluorophenyl)methylcarbamoyl]-2-methyl-phenoxy}-2-pyridyl)-1-methyl-pyrazole-3-carboxamide (86)

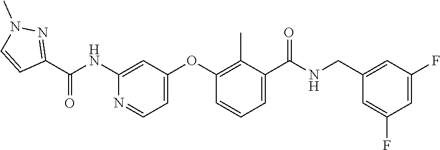 [SMILES: Cc1c(Oc2ccnc(NC(=O)c3ccn(C)n3)c2)cccc1C(=O)NCc1cc(F)cc(F)c1]

Compound 86 was synthesized from intermediate 67b (0.09 mmol) and 3,5-difluorobenzylamine (0.13 mmol) as a white solid in 49% yield according to the general method C2. ¹H NMR (400 MHz, DMSO-d₆) δ (ppm): 9.53 (s, 1H), 9.03 (t, J=6.0 Hz, 1H), 8.23 (d, J=5.8 Hz, 1H), 7.87 (d, J=2.3 Hz, 1H), 7.71 (d, J=2.3 Hz, 1H), 7.44-7.35 (m, 2H), 7.26 (dd, J=7.2 Hz, 2.1 Hz, 1H), 7.17-7.02 (m, 3H), 6.81 (d, J=2.3 Hz, 1H), 6.65 (dd, J=5.7 Hz, 2.4 Hz, 1H), 4.47 (d, J=6.0 Hz, 2H), 3.95 (s, 3H), 2.14 (s, 3H).

ESI-MS: 478.15 (M+H)⁺.

### N-(4-{3-[(6-methoxy-3-pyridyl)methylcarbamoyl]-2-methyl-phenoxy}-2-pyridyl)-1-methyl-pyrazole-3-carboxamide (87)

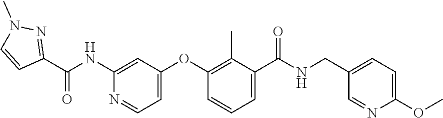 [SMILES: COc1ccc(CNC(=O)c2cccc(Oc3ccnc(NC(=O)c4ccn(C)n4)c3)c2C)cn1]

Compound 87 was synthesized from intermediate 67b (0.09 mmol) and (6-methoxypyridin-3-yl) methanamine (0.13 mmol) as a white solid in 45% yield according to the general method C2. ¹H NMR (400 MHz, DMSO-d₆) δ (ppm): 9.52 (s, 1H), 8.93 (t, J=5.9 Hz, 1H), 8.22 (d, J=5.7 Hz, 1H), 8.14 (d, J=2.0 Hz, 1H), 7.87 (d, J=2.3 Hz, 1H), 7.71-7.69 (m, 2H), 7.38 (t, J=7.7 Hz, 1H), 7.32 (dd, J=7.6 Hz, 1.3 Hz, 1H), 7.24 (dd, J=7.9 Hz, 1.2 Hz, 1H), 6.82-6.80 (m, 2H), 6.64 (dd, J=5.7 Hz, 2.4 Hz, 1H), 4.38 (d, J=5.9 Hz, 2H), 3.95 (s, 3H), 3.83 (s, 3H), 2.12 (s, 3H).

ESI-MS: 473.15 (M+H)⁺.

### 1-methyl-N-[4-(2-methyl-3-{[6-(trifluoromethyl)-3-pyridyl]methylcarbamoyl} phenoxy)-2-pyridyl]pyrazole-3-carboxamide (88)

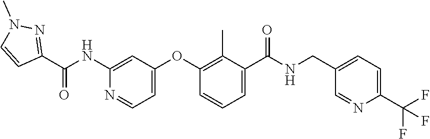 [SMILES: Cc1c(Oc2ccnc(NC(=O)c3ccn(C)n3)c2)cccc1C(=O)NCc1ccc(C(F)(F)F)nc1]

Compound 88 was synthesized from intermediate 67b (0.09 mmol) and [6-(trifluoromethyl)-3-pyridyl]methanamine (0.13 mmol) as a white solid in 48% yield according to the general method C2. ¹H NMR (400 MHz, DMSO-d₆) δ (ppm): 9.54 (s, 1H), 9.13 (t, J=5.9 Hz, 1H), 8.77 (d, J=1.2 Hz, 1H), 8.23 (d, J=5.7 Hz, 1H), 8.05 (dd, J=8.1 Hz, 1.4 Hz, 1H), 7.91 (d, J=8.1 Hz, 1H), 7.87 (d, J=2.3 Hz, 1H), 7.70 (d, J=2.3 Hz, 1H), 7.44-7.36 (m, 2H), 7.31-7.24 (m, 1H), 6.81 (d, J=2.3 Hz, 1H), 6.66 (dd, J=5.7 Hz, 2.4 Hz, 1H), 4.58 (d, J=5.8 Hz, 2H), 3.95 (s, 3H), 2.14 (s, 3H).

ESI-MS: 511.15 (M+H)⁺.

### N-(4-{3-[(3-fluorophenyl)methylcarbamoyl]-2-methyl-phenoxy}-2-pyridyl)-2-methyl-pyrazole-3-carboxamide (89)

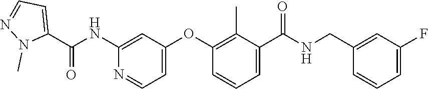 [SMILES: Cc1c(Oc2ccnc(NC(=O)c3ccnn3C)c2)cccc1C(=O)NCc1cccc(F)c1]

Compound 89 was synthesized from intermediate 67c (0.09 mmol) and 3-fluorobenzylamine (0.13 mmol) as a white solid in 33% yield according to the general method C2. ¹H NMR (600 MHz, DMSO-d₆) δ (ppm): 10.85 (s, 1H), 9.00 (t, J=6.1 Hz, 1H), 8.27 (d, J=5.8 Hz, 1H), 7.72 (d, J=2.2 Hz, 1H), 7.50 (d, J=2.1 Hz, 1H), 7.41-7.35 (m, 3H), 7.26-7.24 (m, 2H), 7.20 (d, J=7.6 Hz, 1H), 7.16 (d, J=10.2 Hz, 1H), 7.09 (td, J=8.3 Hz, 2.0 Hz, 1H), 6.65 (dd, J=5.7 Hz, 2.4 Hz, 1H), 4.47 (d, J=6.0 Hz, 2H), 4.05 (s, 3H), 2.15 (s, 3H).

ESI-MS: 460.20 (M+H)⁺.

### N-(4-{3-[(4-fluorophenyl)methylcarbamoyl]-2-methyl-phenoxy}-2-pyridyl)-2-methyl-pyrazole-3-carboxamide (90)

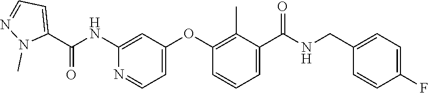 [SMILES: Cc1c(Oc2ccnc(NC(=O)c3ccnn3C)c2)cccc1C(=O)NCc1ccc(F)cc1]

Compound 90 was synthesized from intermediate 67c (0.09 mmol) and 4-fluorobenzylamine (0.13 mmol) as a white solid in 32% yield according to the general method C2. ¹H NMR (600 MHz, DMSO-d₆) δ (ppm): 10.85 (s, 1H), 8.96 (t, J=6.1 Hz, 1H), 8.29-8.24 (m, 1H), 7.72 (d, J=2.2 Hz, 1H), 7.50 (d, J=2.1 Hz, 1H), 7.41-7.37 (m, 3H), 7.33 (dd, J=7.6 Hz, 1.1 Hz, 1H), 7.27-7.23 (m, 2H), 7.20-7.14 (m, 2H), 6.64 (dd, J=5.7 Hz, 2.4 Hz, 1H), 4.43 (d, J=6.0 Hz, 2H), 4.05 (s, 3H), 2.14 (s, 3H).

ESI-MS: 460.20 (M+H)⁺.

### N-(4-{3-[(3-chlorophenyl)methylcarbamoyl]-2-methyl-phenoxy}-2-pyridyl)-2-methyl-pyrazole-3-carboxamide (91)

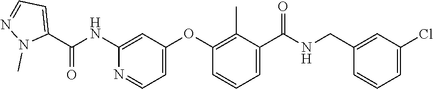 [SMILES: Cc1c(Oc2ccnc(NC(=O)c3ccnn3C)c2)cccc1C(=O)NCc1cccc(Cl)c1]

Compound 91 was synthesized from intermediate 67c (0.09 mmol) and 3-chlorobenzylamine (0.13 mmol) as a white solid in 37% yield according to the general method C2. ¹H NMR (600 MHz, DMSO-d₆) δ (ppm): 10.85 (s, 1H), 9.00 (t, J=6.0 Hz, 1H), 8.31-8.23 (m, 1H), 7.72 (d, J=2.3, 1H), 7.50 (d, J=2.1 Hz, 1H), 7.43-7.30 (m, 6H), 7.25 (m, 2H), 6.65 (dd, J=5.7 Hz, 2.4 Hz, 1H), 4.46 (d, J=6.0 Hz, 2H), 4.05 (s, 3H), 2.15 (s, 3H).

ESI-MS: 476.15 (M+H)⁺.

### N-(4-{3-[(4-chlorophenyl)methylcarbamoyl]-2-methyl-phenoxy}-2-pyridyl)-2-methyl-pyrazole-3-carboxamide (92)

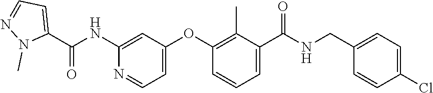 [SMILES: Cc1c(Oc2ccnc(NC(=O)c3ccnn3C)c2)cccc1C(=O)NCc1ccc(Cl)cc1]

Compound 92 was synthesized from intermediate 67c (0.09 mmol) and 4-chlorobenzylamine (0.13 mmol) as a white solid in 35% yield according to the general method C2. ¹H NMR (600 MHz, DMSO-d₆) δ (ppm): 10.85 (s, 1H), 8.98 (t, J=6.1 Hz, 1H), 8.29-8.24 (m, 1H), 7.72 (d, J=2.2 Hz, 1H), 7.50 (d, J=2.1 Hz, 1H), 7.42-7.33 (m, 5H), 7.26 (d, J=2.1 Hz, 2H), 7.25 (dd, J=8.0 Hz, 1.1 Hz, 1H) 6.64 (dd, J=5.7 Hz, 2.4 Hz, 1H), 4.44 (d, J=6.0 Hz, 2H), 4.05 (s, 3H), 2.14 (s, 3H).

ESI-MS: 476.15 (M+H)⁺.

### N-{4-[3-(imidazo[1,2-a]pyridin-6-ylmethylcarbamoyl)-2-methyl-phenoxy]-2-pyridyl}-2-methyl-pyrazole-3-carboxamide (93)

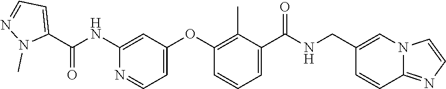 [SMILES: Cc1c(Oc2ccnc(NC(=O)c3ccnn3C)c2)cccc1C(=O)NCc1ccc2nccn2c1]

Compound 93 was synthesized from intermediate 67c (0.09 mmol) and imidazo[1,2-a]pyridin-6-ylmethanamine (0.13 mmol) as a white solid in 15% yield according to the general method C2. ¹H NMR (600 MHz, DMSO-d₆) δ (ppm): 10.85 (s, 1H), 8.98 (t, J=6.0 Hz, 1H), 8.52-8.49 (m, 1H), 8.26 (d, J=5.7 Hz, 1H), 7.98-7.95 (m, 1H), 7.71 (d, J=2.3 Hz, 1H), 7.58-7.54 (m, 2H), 7.50 (d, J=2.1 Hz, 1H), 7.41-7.34 (m, 2H), 7.27-7.22 (m, 3H), 6.64 (dd, J=5.7 Hz, 2.3 Hz, 1H), 4.45 (d, J=5.8 Hz, 2H), 4.04 (s, 3H), 2.14 (s, 3H).

ESI-MS: 482.20 (M+H)⁺.

### N-{4-[3-(imidazo[1,2-a]pyridin-7-ylmethylcarbamoyl)-2-methyl-phenoxy]-2-pyridyl}-2-methyl-pyrazole-3-carboxamide (94)

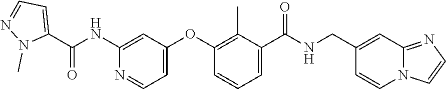 [SMILES: Cc1c(Oc2ccnc(NC(=O)c3ccnn3C)c2)cccc1C(=O)NCc1ccn2ccnc2c1]

Compound 94 was synthesized from intermediate 67c (0.09 mmol) and imidazo[1,2-a]pyridin-7-ylmethanamine (0.13 mmol) as a white solid in 20% yield according to the general method C2. ¹H NMR (600 MHz, DMSO-d₆) δ (ppm): 10.85 (s, 1H), 9.03 (t, J=6.0 Hz, 1H), 8.51 (dd, J=7.0 Hz, 0.9 Hz, 1H), 8.27 (d, J=5.7 Hz, 1H), 7.91-7.88 (m, 1H), 7.72 (d, J=2.3 Hz, 1H), 7.53 (d, J=1.2 Hz, 1H), 7.50 (d, J=2.1 Hz, 1H), 7.45-7.36 (m, 3H), 7.28-7.24 (m, 2H), 6.89 (dd, J=7.0 Hz, 1.7 Hz, 1H), 6.65 (dd, J=5.7 Hz, 2.3 Hz, 1H), 4.49 (d, J=5.8 Hz, 2H), 4.05 (s, 3H), 2.17 (s, 3H).

ESI-MS: 482.20 (M+H)⁺.

### N-(4-{3-[(3,5-difluorophenyl)methylcarbamoyl]-2-methyl-phenoxy}-2-pyridyl)-2-methyl-pyrazole-3-carboxamide (95)

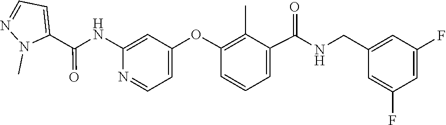 [SMILES: Cc1c(Oc2ccnc(NC(=O)c3ccnn3C)c2)cccc1C(=O)NCc1cc(F)cc(F)c1]

Compound 95 was synthesized from intermediate 67c (0.09 mmol) and 3,5-difluorobenzylamine (0.13 mmol) as a white solid in 42% yield according to the general method C2. ¹H NMR (600 MHz, DMSO-d₆) δ (ppm): 10.85 (s, 1H), 9.02 (t, J=6.1 Hz, 1H), 8.27 (d, J=5.7 Hz, 1H), 7.72 (d, J=2.3 Hz, 1H), 7.50 (d, J=2.1 Hz, 1H), 7.43-7.36 (m, 2H), 7.27-7.25 (m, 2H), 7.12 (tt, J=9.3 Hz, 2.4 Hz, 1H), 7.08-7.04 (m, 2H), 6.65 (dd, J=5.5 Hz, 2.3 Hz, 1H), 4.47 (d, J=6.0 Hz, 2H), 4.05 (s, 3H), 2.15 (s, 3H).

ESI-MS: 478.15 (M+H)⁺.

### N-(4-{3-[(6-methoxy-3-pyridyl)methylcarbamoyl]-2-methyl-phenoxy}-2-pyridyl)-2-methyl-pyrazole-3-carboxamide (96)

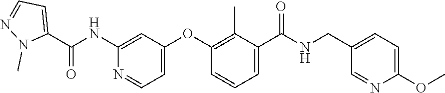 [SMILES: COc1ccc(CNC(=O)c2cccc(Oc3ccnc(NC(=O)c4ccnn4C)c3)c2C)cn1]

Compound 96 was synthesized from intermediate 67c (0.09 mmol) and (6-methoxypyridin-3-yl) methanamine (0.13 mmol) as a white solid in 38% yield according to the general method C2. ¹H NMR (600 MHz, DMSO-d₆) δ (ppm): 10.85 (s, 1H), 8.92 (t, J=5.9 Hz, 1H), 8.33-8.22 (m, 1H), 8.14 (dd, J=2.4 Hz, 0.6 Hz, 1H), 7.76-7.64 (m, 2H), 7.50 (d, J=2.1 Hz, 1H), 7.39-7.36 (m, 1H), 7.31 (dd, J=7.6 Hz, 1.1 Hz, 1H), 7.26-7.22 (m, 2H), 6.81 (dd, J=8.5 Hz, 0.6 Hz, 1H), 6.63 (dd, J=5.7 Hz, 2.4 Hz, 1H), 4.38 (d, J=5.9 Hz, 2H), 4.05 (s, 3H), 3.83 (s, 3H), 2.12 (s, 3H).

ESI-MS: 473.20 (M+H)⁺.

### 2-methyl-N-[4-(2-methyl-3-{[6-(trifluoromethyl)-3-pyridyl]methylcarbamoyl} phenoxy)-2-pyridyl]pyrazole-3-carboxamide (97)

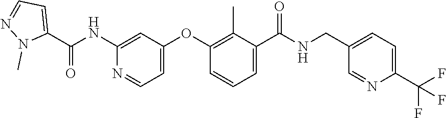 [SMILES: Cc1c(Oc2ccnc(NC(=O)c3ccnn3C)c2)cccc1C(=O)NCc1ccc(C(F)(F)F)nc1]

Compound 97 was synthesized from intermediate 67c (0.09 mmol) and [6-(trifluoromethyl)-3-pyridyl]methanamine (0.13 mmol) as a white solid in 44% yield according to the general method C2. ¹H NMR (400 MHz, DMSO-d₆) δ (ppm): 10.88 (s, 1H), 9.12 (t, J=5.9 Hz, 1H), 8.76 (s, 1H), 8.27 (d, J=5.7 Hz, 1H), 8.05 (d, J=9.3 Hz, 1H), 7.91 (d, J=8.1 Hz, 1H), 7.71 (d, J=2.3 Hz, 1H), 7.50 (d, J=2.1 Hz, 1H), 7.44-7.36 (m, 2H), 7.28-7.25 (m, 2H), 6.65 (dd, J=5.7 Hz, 2.3 Hz, 1H), 4.58 (d, J=5.8 Hz, 2H), 4.04 (s, 3H), 2.14 (s, 3H).

ESI-MS: 511.15 (M+H)⁺.

### N-(4-{3-[(5-fluoro-6-methoxy-3-pyridyl)methylcarbamoyl]-2-methyl-phenoxy}-2-pyridyl)-2-methyl-pyrazole-3-carboxamide (98)

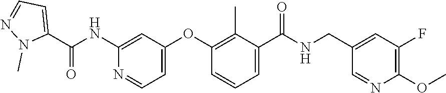 [SMILES: COc1ncc(CNC(=O)c2cccc(Oc3ccnc(NC(=O)c4ccnn4C)c3)c2C)cc1F]

Compound 98 was synthesized from intermediate 67c (0.09 mmol) and (5-fluoro-6-methoxy-3-pyridyl) methanamine (0.13 mmol) as a white solid in 36% yield according to the general method C2. ¹H NMR (400 MHz, DMSO-d₆) δ (ppm): 10.86 (s, 1H), 8.95 (t, J=5.8 Hz, 1H), 8.27 (d, J=5.7 Hz, 1H), 7.98 (d, J=1.7 Hz, 1H), 7.71 (d, J=2.3 Hz, 1H), 7.65 (dd, J=11.4 Hz, 1.9 Hz, 1H), 7.50 (d, J=2.1 Hz, 1H), 7.44-7.31 (m, 2H), 7.26-7.23 (m, 2H), 6.64 (dd, J=5.7 Hz, 2.3 Hz, 1H), 4.41 (d, J=5.9 Hz, 2H), 4.05 (s, 3H), 3.93 (s, 3H), 2.13 (s, 3H).

ESI-MS: 491.10 (M+H)⁺.

### N-{4-[2-methyl-3-(4-pyridylmethylcarbamoyl)phenoxy]-2-pyridyl}pyridine-3-carboxamide (99)

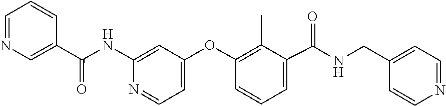 [SMILES: Cc1c(Oc2ccnc(NC(=O)c3cccnc3)c2)cccc1C(=O)NCc1ccncc1]

Compound 99 was synthesized from intermediate 67d (0.13 mmol) and 4-(aminomethyl)pyridine (0.19 mmol) as a white solid in 7% yield according to the general method C2. ¹H NMR (400 MHz, DMSO-d₆) δ (ppm): 11.14 (s, 1H), 9.08-9.05 (m, 2H), 8.73 (dd, J=4.8 Hz, 1.6 Hz, 1H), 8.53 (dd, J=4.4 Hz, 1.5 Hz, 2H), 8.35-8.24 (m, 2H), 7.77 (d, J=2.3 Hz, 1H), 7.52 (dd, J=7.7 Hz, 5.1 Hz, 1H), 7.45-7.39 (m, 2H), 7.35 (d, J=5.9 Hz, 2H), 7.31-7.24 (m, 1H), 6.70 (dd, J=5.7 Hz, 2.4 Hz, 1H), 4.48 (d, J=6.0 Hz, 2H), 2.17 (s, 3H).

ESI-MS: 440.15 (M+H)⁺.

### N-(4-{3-[(2,6-difluoro-4-pyridyl)methylcarbamoyl]-2-methyl-phenoxy}-2-pyridyl) pyridine-3-carboxamide (100)

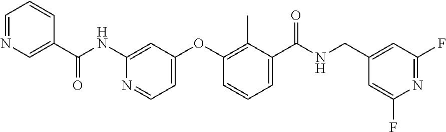 [SMILES: Cc1c(Oc2ccnc(NC(=O)c3cccnc3)c2)cccc1C(=O)NCc1cc(F)nc(F)c1]

Compound 100 was synthesized from intermediate 67d (0.13 mmol) and (2,6-difluoro-4-pyridyl) methanamine (0.38 mmol) as a white solid in 14% yield according to the general method C2. ¹H NMR (400 MHz, DMSO-d₆) δ (ppm): 11.14 (s, 1H), 9.12 (t, J=5.8 Hz, 1H), 9.08 (d, J=1.7 Hz, 1H), 8.73 (dd, J=4.8 Hz, 1.5 Hz, 1H), 8.34-8.24 (m, 2H), 7.76 (d, J=2.2 Hz, 1H), 7.52 (dd, J=7.5 Hz, 4.8 Hz, 1H), 7.48-7.40 (m, 2H), 7.28 (dd, J=7.6 Hz, 1.4 Hz, 1H), 7.13 (s, 2H), 6.71 (dd, J=5.7 Hz, 2.3 Hz, 1H), 4.57 (d, J=5.9 Hz, 2H), 2.17 (s, 3H).

ESI-MS: 476.20 (M+H)⁺.

### N-(4-{3-[(4-cyano-3-fluoro-phenyl)methylcarbamoyl]-2-methyl-phenoxy}-2-pyridyl)pyridine-3-carboxamide (101)

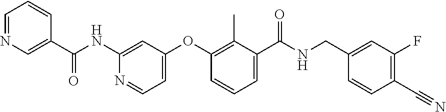 [SMILES: Cc1c(Oc2ccnc(NC(=O)c3cccnc3)c2)cccc1C(=O)NCc1ccc(C#N)c(F)c1]

Compound 101 was synthesized from intermediate 67d (0.13 mmol) and 4-aminomethyl-2-fluorobenzonitrile (0.14 mmol) as a white solid in 8% yield according to the general method C2. ¹H NMR (400 MHz, DMSO-d₆) δ (ppm): 1H NMR (400 MHz, DMSO) δ 11.14 (s, 1H), 9.15-9.05 (m, 2H), 8.73 (dd, J=4.7 Hz, 1.5 Hz, 1H), 8.34-8.26 (m, 2H), 7.97-7.88 (m, 1H), 7.76 (d, J=2.2 Hz, 1H), 7.55-7.38 (m, 5H), 7.30-7.25 (m, 1H), 6.70 (dd, J=5.7 Hz, 2.3 Hz, 1H), 4.55 (d, J=5.9 Hz, 2H), 2.16 (s, 3H).

ESI-MS: 482.15 (M+H)⁺.

### Example 3: General Procedure for the Synthesis of Analogues 103-105

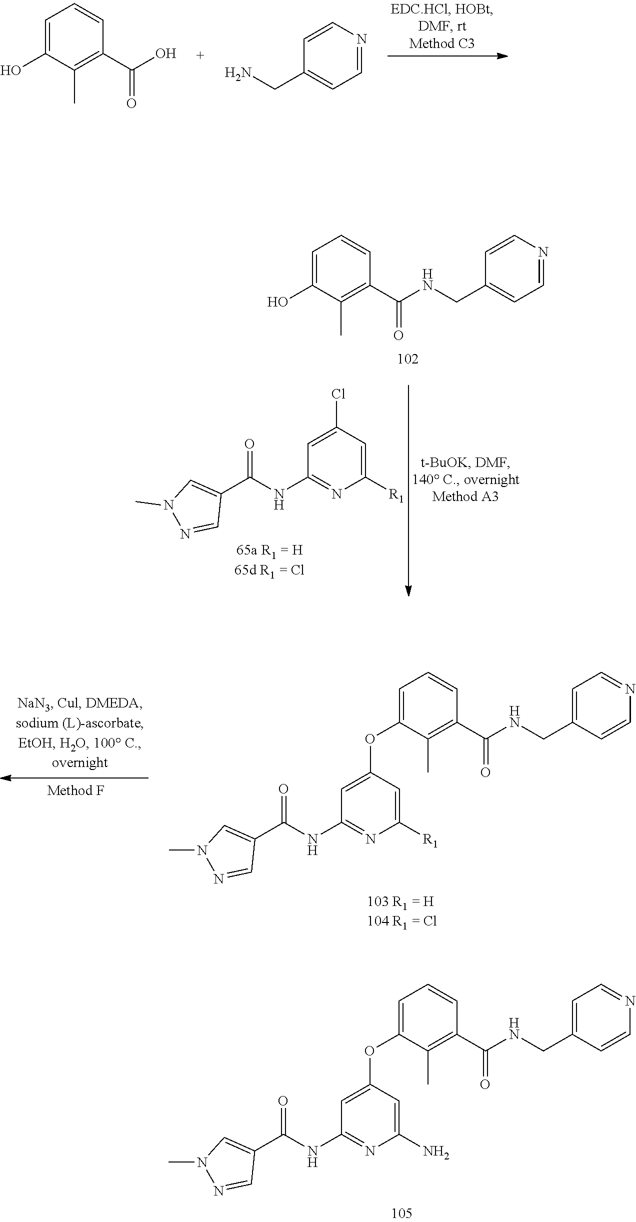

### Preparation of 3-hydroxy-2-methyl-N-(4-pyridylmethyl)benzamide (102)

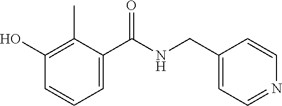 [SMILES: Cc1c(O)cccc1C(=O)NCc1ccncc1]

Intermediate 102 was synthesized from 3-hydroxy-2-methylbenzoic acid (19.6 mmol) and 4-(aminomethyl)pyridine (19.6 mmol) as a white solid in 93% yield according to the general method C3.
Method A3: To a solution of phenol derivative (1 equiv.) in DMF (5 mL/mmol) under nitrogen was added t-BuOK (1.5 equiv.). 4-Chloropyridine derivative (1 equiv.) was added and the reaction mixture was stirred at 140° C. until completion (from 24 to 48 hours). The reaction mixture was concentrated under reduced pressure. The crude was purified by flash column chromatography (DCM/MeOH from 100/0 to 90/10) and reverse phase chromatography (H₂O/MeOH from 100/0 to 0/100) to give the expected compound.
Method F: To a stirred solution of 104 (30 mg, 0.063 mmol) in EtOH/H₂O (0.75 mL/0.25 mL) were added sodium (L)-ascorbate (2 mg, 0.006 mmol), sodium azide (9 mg, 0.126 mmol), copper iodide (3 mg, 0.013 mmol) and DMEDA (2 μL, 0.019 mmol). The reaction mixture was stirred at 100° C. overnight. The reaction mixture was concentrated under reduced pressure. The crude was purified by flash column chromatography (DCM/MeOH from 100/0 to 80/20) and reverse phase chromatography (H₂O/MeOH: 0 to 100%) to give 6 mg of N-{6-amino-4-[2-methyl-3-(4-pyridylmethylcarbamoyl)phenoxy]-2-pyridyl}-1-methyl-pyrazole-4-carboxamide 105 in 21% yield. ¹H NMR (500 MHz, DMSO-d₆) δ (ppm): 9.94 (s, 1H), 9.05 (t, J=6.1 Hz, 1H), 8.54-8.52 (m, 2H), 8.36 (s, 1H), 8.05 (d, J=0.6 Hz, 1H), 7.40-7.29 (m, 4H), 7.19 (dd, J=7.0 Hz, 2.3 Hz, 1H), 7.07 (d, J=2.0 Hz, 1H), 5.79 (bs, 2H), 5.56 (d, J=2.0 Hz, 1H), 4.47 (d, J=6.0 Hz, 2H), 3.85 (s, 3H), 2.16 (s, 3H).

ESI-MS: 458.15 (M+H)⁺.

The following compounds are examples illustrating Method A3:

### 1-methyl-N-{4-[2-methyl-3-(4-pyridylmethylcarbamoyl)phenoxy]-2-pyridyl} pyrazole-4-carboxamide (103)

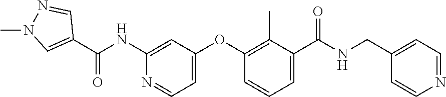 [SMILES: Cc1c(Oc2ccnc(NC(=O)c3cnn(C)c3)c2)cccc1C(=O)NCc1ccncc1]

Compound 103 was synthesized from intermediate 102 (0.22 mmol) and 65a (0.22 mmol) as a white solid in 19% yield according to the general method A3. ¹H NMR (400 MHz, DMSO-d₆) δ (ppm): 10.53 (s, 1H), 9.05 (t, J=6.0 Hz, 1H), 8.53 (dd, J=4.5 Hz, 1.5 Hz, 2H), 8.40 (s, 1H), 8.23 (d, J=5.7 Hz, 1H), 8.09 (s, 1H), 7.75 (d, J=2.3 Hz, 1H), 7.42-7.33 (m, 4H), 7.28-7.20 (m, 1H), 6.62 (dd, J=5.7 Hz, 2.4 Hz, 1H), 4.48 (d, J=6.0 Hz, 2H), 3.86 (s, 3H), 2.16 (s, 3H).

### N-{6-chloro-4-[2-methyl-3-(4-pyridylmethylcarbamoyl)phenoxy]-2-pyridyl}-1-methyl-pyrazole-4-carboxamide (104)

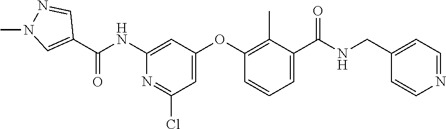 [SMILES: Cc1c(Oc2cc(Cl)nc(NC(=O)c3cnn(C)c3)c2)cccc1C(=O)NCc1ccncc1]

Compound 104 was synthesized from intermediate 102 (0.22 mmol) and 65d (0.22 mmol) as a white solid in 23% yield according to the general method A3. ¹H NMR (500 MHz, DMSO-d₆) δ (ppm): 10.84 (s, 1H), 9.10 (t, J=6.1 Hz, 1H), 8.53 (dd, J=4.4 Hz, 1.6 Hz, 2H), 8.41 (s, 1H), 8.10 (d, J=0.5 Hz, 1H), 7.73 (d, J=2.0 Hz, 1H), 7.44-7.40 (m, 2H), 7.35 (d, J=6.0 Hz, 2H), 7.33-7.28 (m, 1H), 6.73 (d, J=2.0 Hz, 1H), 4.48 (d, J=6.0 Hz, 2H), 3.85 (s, 3H), 2.16 (s, 3H).

ESI-MS: 477.15 (M+H)⁺.

### Example 4: General Procedure for the Synthesis of Analogues 106-108

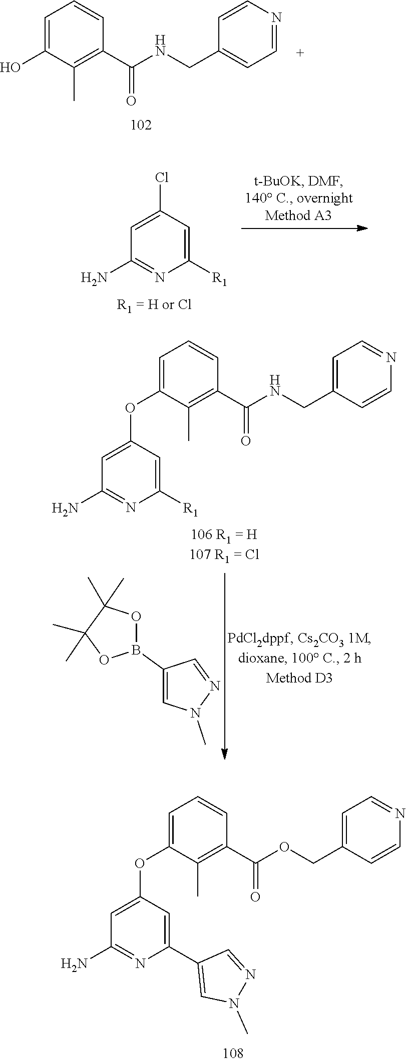

### 3-[(2-amino-4-pyridyl)oxy]-2-methyl-N-(4-pyridylmethyl)benzamide (106)

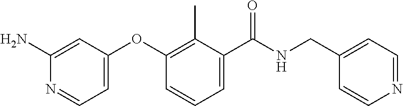 [SMILES: Cc1c(Oc2ccnc(N)c2)cccc1C(=O)NCc1ccncc1]

Compound 106 was synthesized from intermediate 102 (0.39 mmol) and 2-amino-4-chloropyridine (0.39 mmol) as a white solid in 23% yield according to the general method A3. ¹H NMR (400 MHz, DMSO-d₆) δ (ppm): 8.99 (t, J=6.0 Hz, 1H), 8.54-8.52 (m, 2H), 7.79 (d, J=5.8 Hz, 1H), 7.36-7.33 (m, 4H), 7.17 (dd, J=6.4 Hz, 2.9 Hz, 1H), 6.08 (dd, J=5.8 Hz, 2.3 Hz, 1H), 5.92 (s, 2H), 5.74 (d, J=2.2 Hz, 1H), 4.47 (d, J=6.0 Hz, 2H), 2.13 (s, 3H).

### 3-[(2-amino-4-pyridyl)oxy]-2-methyl-N-(4-pyridylmethyl)benzamide (107)

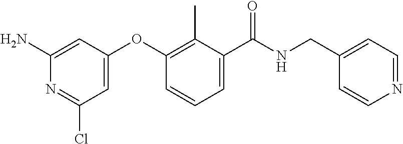 [SMILES: Cc1c(Oc2cc(N)nc(Cl)c2)cccc1C(=O)NCc1ccncc1]

Compound 107 was synthesized from intermediate 102 (0.61 mmol) and 2-amino-4,6-dichloropyridine (0.61 mmol) as a white solid in 30% yield according to the general method A3. ¹H NMR (400 MHz, DMSO-d₆) δ (ppm): 9.05 (t, J=6.0 Hz, 1H), 8.53 (d, J=5.4 Hz, 2H), 7.40-7.31 (m, 4H), 7.25-7.20 (m, 1H), 6.45 (s, 2H), 6.13 (d, J=1.9 Hz, 1H), 5.65 (d, J=1.9 Hz, 1H), 4.47 (d, J=6.0 Hz, 2H), 2.12 (s, 3H).

### 3-{[2-amino-6-(1-methylpyrazol-4-yl)-4-pyridyl]oxy}-2-methyl-N-(4-pyridyl methyl)benzamide (108)

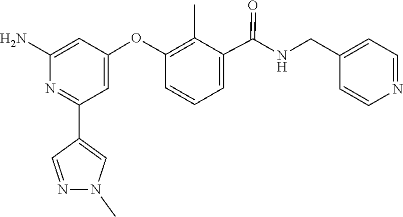 [SMILES: Cc1c(Oc2cc(N)nc(-c3cnn(C)c3)c2)cccc1C(=O)NCc1ccncc1]

Method D3: To a stirred solution of 107 (32 mg, 0.086 mmol) in dioxane (1 mL) under nitrogen were added PdCl₂dppf (7 mg, 0.009 mmol), 1-methyl-4-(4,4,5,5-tetramethyl-1,3,2-dioxaborolan-1H-pyrazole (27 mg, 0.13 mmol) and Cs₂CO₃ 1M (0.215 mL, 0.215 mmol). The reaction mixture was stirred at 100° C. for 2 h. The reaction mixture was concentrated under reduced pressure. The crude was purified by flash column chromatography (DCM/MeOH) and reverse phase chromatography (H₂O/MeOH: 0 to 100%) to give 18 mg of 108 in 51% yield. 1H NMR (400 MHz, DMSO-d₆) δ (ppm): 9.03 (t, J=6.0 Hz, 1H), 8.54-8.52 (m 2H), 8.06 (s, 1H), 7.83 (d, J=0.6 Hz, 1H), 7.41-7.32 (m, 4H), 7.20 (dd, J=6.7 Hz, 2.6 Hz, 1H), 6.51 (d, J=2.0 Hz, 1H), 5.92 (s, 2H), 5.49 (d, J=2.0 Hz, 1H), 4.47 (d, J=6.0 Hz, 2H), 3.84 (s, 3H), 2.16 (s, 3H).

### Example 5: General Procedure for the Synthesis of Analogues 110-116

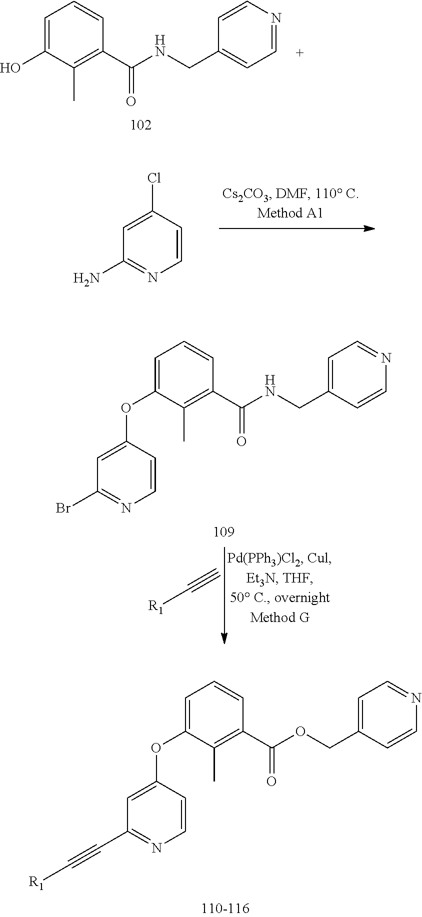

### Preparation of 3-[(2-bromo-4-pyridyl)oxy]-2-methyl-N-(4-pyridylmethyl)benzamide (109)

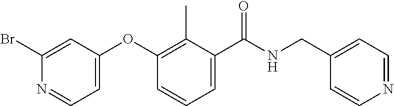 [SMILES: Cc1c(Oc2ccnc(Br)c2)cccc1C(=O)NCc1ccncc1]

Intermediate 109 was synthesized from intermediate 102 (5.87 mmol) and 2-bromo-4-chloropyridine (5.87 mmol) as a white solid in 84% yield according to the general method A1.

ESI-MS: 398.10-400.10 (M+H)⁺.
Method G: To a solution of 109 (1 equiv.) in THF (20 mL/mmol) under nitrogen were added alkyne derivative (3 equiv.), Pd(PPh₃)Cl₂ (0.1 equiv.), CuI (0.2 equiv.) and triethylamine (3 equiv.). The mixture was stirred at 50° C. overnight. The reaction mixture was concentrated under reduced pressure. The crude was purified by flash column chromatography (DCM/MeOH from 100/0 to 90/10) and reverse phase chromatography (H₂O/MeOH from 100/0 to 0/100) to give the expected compound.

### 2-methyl-3-{[2-(3-phenylprop-1-ynyl)-4-pyridyl]oxy}-N-(4-pyridylmethyl)benzamide (110)

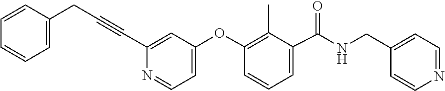 [SMILES: Cc1c(Oc2ccnc(C#CCc3ccccc3)c2)cccc1C(=O)NCc1ccncc1]

Compound 110 was synthesized from intermediate 109 (0.10 mmol) and 3-phenyl-1-propyne (0.30 mmol) as a white solid in 21% yield according to the general method G. ¹H NMR (400 MHz, DMSO-d₆) δ (ppm): 9.03 (t, J=6.0 Hz, 1H), 8.55-8.52 (m, 2H), 8.41 (d, J=5.7 Hz, 1H), 7.44-7.32 (m, 9H), 7.28-7.23 (m, 1H), 6.89 (d, J=2.4 Hz, 1H), 6.85 (dd, J=5.7 Hz, 2.5 Hz, 1H), 4.48 (d, J=6.0 Hz, 2H), 3.90 (s, 2H), 2.12 (s, 3H).

ESI-MS: 434.25 (M+H)⁺.

### 3-{[2-(3-hydroxyprop-1-ynyl)-4-pyridyl]oxy}-2-methyl-N-(4-pyridylmethyl)benzamide (111)

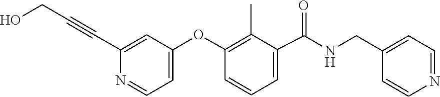 [SMILES: Cc1c(Oc2ccnc(C#CCO)c2)cccc1C(=O)NCc1ccncc1]

Compound 111 was synthesized from intermediate 109 (0.10 mmol) and propargyl alcohol (0.30 mmol) as a white solid in 22% yield according to the general method G. ¹H NMR (400 MHz, DMSO-d₆) δ (ppm): 9.05 (t, J=6.0 Hz, 1H), 8.54-8.52 (m, 2H), 8.43 (d, J=5.7 Hz, 1H), 7.44-7.39 (m, 2H), 7.34 (d, J=6.0 Hz, 2H), 7.29-7.23 (m, 1H), 6.91 (dd, J=5.7 Hz, 2.5 Hz, 1H), 6.80 (d, J=2.4 Hz, 1H), 5.41 (bs, 1H), 4.48 (d, J=6.0 Hz, 2H), 4.28 (s, 2H), 2.11 (s, 3H).

ESI-MS: 374.20 (M+H)⁺.

### 3-{[2-(3-aminoprop-1-ynyl)-4-pyridyl]oxy}-2-methyl-N-(4-pyridylmethyl)benzamide (112)

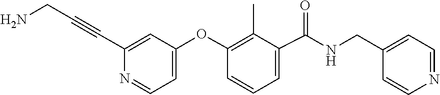 [SMILES: Cc1c(Oc2ccnc(C#CCN)c2)cccc1C(=O)NCc1ccncc1]

Compound 112 was synthesized from intermediate 109 (0.10 mmol) and propargylamine (0.30 mmol) as a white solid in 22% yield according to the general method G. ¹H NMR (400 MHz, DMSO-d₆) δ (ppm): 9.05 (t, J=6.0 Hz, 1H), 8.54-8.52 (m, 2H), 8.41 (d, J=5.7 Hz, 1H), 7.44-7.39 (m, 2H), 7.34 (d, J=5.9 Hz, 2H), 7.26 (dd, J=8.7 Hz, 4.3 Hz, 1H), 6.90 (dd, J=5.7 Hz, 2.5 Hz, 1H), 6.75 (d, J=2.4 Hz, 1H), 4.48 (d, J=6.0 Hz, 3H), 3.47 (s, 2H), 2.11 (s, 4H).

ESI-MS: 373.20 (M+H)⁺.

### 3-{[2-(3-methoxyprop-1-ynyl)-4-pyridyl]oxy}-2-methyl-N-(4-pyridylmethyl)benzamide (113)

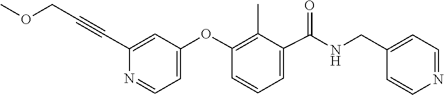 [SMILES: COCC#Cc1cc(Oc2cccc(C(=O)NCc3ccncc3)c2C)ccn1]

Compound 113 was synthesized from intermediate 109 (0.10 mmol) and methyl propargyl ether (0.30 mmol) as a white solid in 31% yield according to the general method G. ¹H NMR (400 MHz, DMSO-d₆) δ (ppm): 9.04 (t, J=5.9 Hz, 1H), 8.54-8.52 (m, 2H), 8.44 (d, J=5.6 Hz, 1H), 7.41 (d, J=4.5 Hz, 2H), 7.35 (d, J=5.7 Hz, 2H), 7.25 (t, J=4.7 Hz, 1H), 6.93-6.86 (m, 2H), 4.48 (d, J=5.9 Hz, 2H), 4.32 (s, 2H), 3.31 (s, 3H), 2.12 (s, 3H).

ESI-MS: 388.20 (M+H)⁺.

### 2-methyl-3-({2-[3-(methylamino) prop-1-ynyl]-4-pyridyl}oxy)-N-(4-pyridylmethyl)benzamide (114)

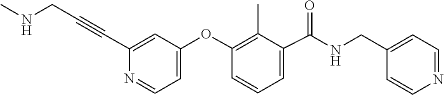 [SMILES: CNCC#Cc1cc(Oc2cccc(C(=O)NCc3ccncc3)c2C)ccn1]

Compound 114 was synthesized from intermediate 109 (0.10 mmol) and N-methyl-N-prop-2-ynylamine (0.30 mmol) as a white solid in 18% yield according to the general method G. ¹H NMR (400 MHz, DMSO-d₆) δ (ppm): 9.05 (t, J=5.9 Hz, 1H), 8.54-8.52 (m, 2H), 8.41 (d, J=5.8 Hz, 1H), 7.41-7.38 (m, 3H), 7.34 (d, J=5.8 Hz, 2H), 7.25 (t, J=4.7 Hz, 1H), 6.87 (dd, J=5.7 Hz, 2.5 Hz, 1H), 6.80 (d, J=2.4 Hz, 1H), 4.48 (d, J=5.9 Hz, 2H), 3.48 (d, J=5.7 Hz, 2H), 2.30 (d, J=5.0 Hz, 3H), 2.11 (s, 3H).

ESI-MS: 387.25 (M+H)⁺.

### 3-{[2-(3-imidazol-1-ylprop-1-ynyl)-4-pyridyl]oxy}-2-methyl-N-(4-pyridylmethyl)benzamide (115)

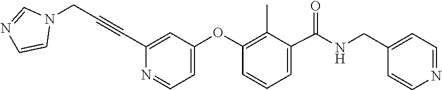 [SMILES: Cc1c(Oc2ccnc(C#CCn3ccnc3)c2)cccc1C(=O)NCc1ccncc1]

Compound 115 was synthesized from intermediate 109 (0.20 mmol) and 1-(2-propyn-1-yl)-1H-imidazole (0.40 mmol) as a white solid in 4% yield according to the general method G. ¹H NMR (400 MHz, DMSO-d₆) δ (ppm): 9.04 (t, J=5.9 Hz, 1H), 8.54-8.52 (m, 2H), 8.44 (d, J=5.7 Hz, 1H), 7.73 (s, 1H), 7.41 (d, J=4.3 Hz, 2H), 7.34 (d, J=5.8 Hz, 2H), 7.27-7.23 (m, 2H), 6.94-6.93 (m, 2H), 6.89 (dd, J=5.7 Hz, 2.5 Hz, 1H), 5.19 (s, 2H), 4.48 (d, J=5.9 Hz, 2H), 2.11 (s, 3H).

ESI-MS: 424.30 (M+H)⁺.

### 2-methyl-3-({2-[3-(methylamino) prop-1-ynyl]-4-pyridyl}oxy)-N-(4-pyridylmethyl)benzamide (116)

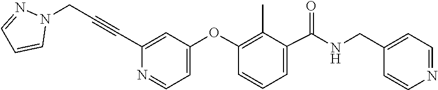 [SMILES: Cc1c(Oc2ccnc(C#CCn3cccn3)c2)cccc1C(=O)NCc1ccncc1]

Compound 116 was synthesized from intermediate 109 (0.20 mmol) and 1-(2-propyn-1-yl)-1H-pyrazole (0.30 mmol) as a white solid in 12% yield according to the general method G. ¹H NMR (400 MHz, DMSO-d₆) δ (ppm): 9.04 (t, J=6.0 Hz, 1H), 8.54-8.52 (m, 2H), 8.46-8.41 (m, 1H), 7.83 (d, J=1.8 Hz, 1H), 7.49 (d, J=1.2 Hz, 1H), 7.40 (d, J=4.2 Hz, 2H), 7.34 (d, J=6.0 Hz, 2H), 7.25 (t, J=4.7 Hz, 1H), 6.98-6.88 (m, 2H), 6.30-6.29 (m, 1H), 5.30 (s, 2H), 4.48 (d, J=6.0 Hz, 2H), 2.11 (s, 3H).

ESI-MS: 424.25 (M+H)⁺.

### Example 6: General Procedure for the Synthesis of Analogues 120-197, 350-355, and 359

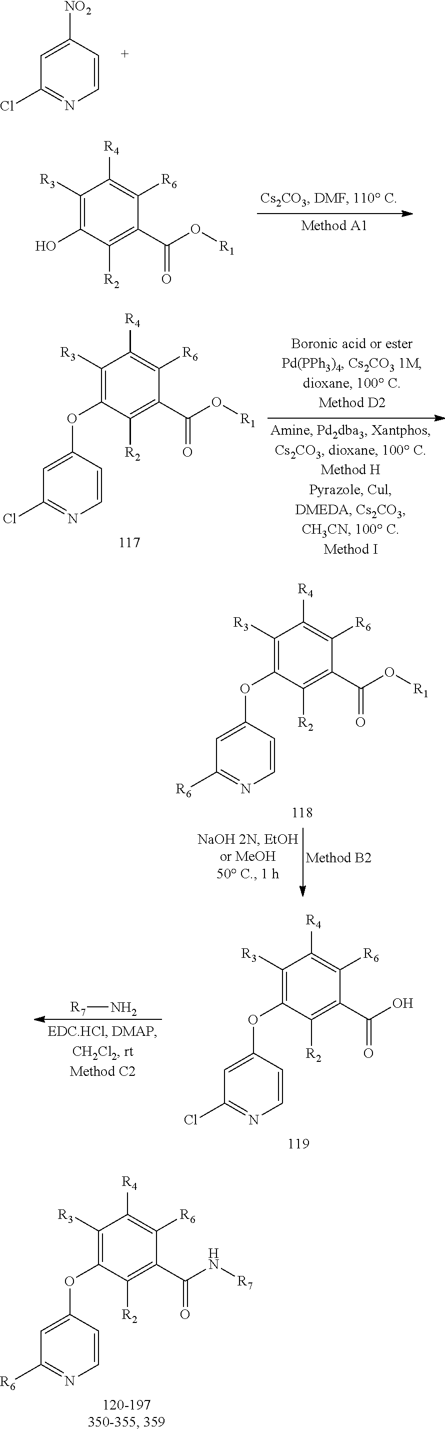

The following compound 117a is an example illustrating Method A1:

### Preparation of ethyl 3-[(2-chloro-4-pyridyl)oxy]-2-methyl-benzoate (117a)

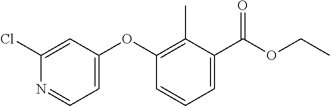 [SMILES: CCOC(=O)c1cccc(Oc2ccnc(Cl)c2)c1C]

Intermediate 117a was synthesized from ethyl 3-hydroxy-2-methyl-benzoate (6.30 mmol) and 2-chloro-4-nitropyridine (6.30 mmol) as a colorless oil in 95% yield according to the general method A1.

ESI-MS: 292.00 (M+H)⁺.

The following table illustrates intermediates 117 prepared from method A1:

| Intermediate | Structure | Synthesis procedure |
|---|---|---|
| Compound 117a | 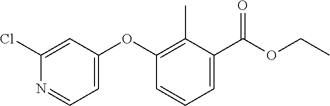 [SMILES: CCOC(=O)c1cccc(Oc2ccnc(Cl)c2)c1C] | Method A1 |
| Compound 117b | 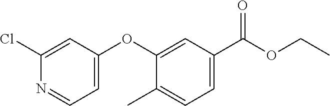 [SMILES: CCOC(=O)c1ccc(C)c(Oc2ccnc(Cl)c2)c1] | Method A1 |
| Compound 117c | 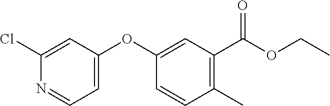 [SMILES: CCOC(=O)c1cc(Oc2ccnc(Cl)c2)ccc1C] | Method A1 |
| Compound 117d | 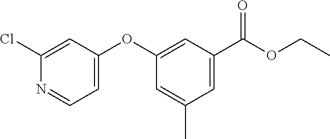 [SMILES: CCOC(=O)c1cc(C)cc(Oc2ccnc(Cl)c2)c1] | Method A1 |
| Compound 117e | 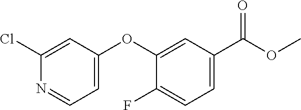 [SMILES: COC(=O)c1ccc(F)c(Oc2ccnc(Cl)c2)c1] | Method A1 |
| Compound 117f | 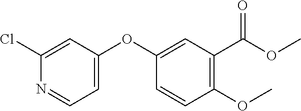 [SMILES: COC(=O)c1cc(Oc2ccnc(Cl)c2)ccc1OC] | Method A1 |
| Compound 117g | 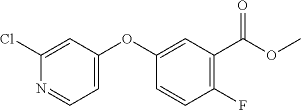 [SMILES: COC(=O)c1cc(Oc2ccnc(Cl)c2)ccc1F] | Method A1 |

The following compound 118a is an example illustrating Method D2:

### Preparation of ethyl 2-methyl-3-{[2-(1-methylpyrazol-4-yl)-4-pyridyl]oxy}benzoate (118a)

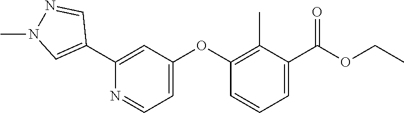 [SMILES: CCOC(=O)c1cccc(Oc2ccnc(-c3cnn(C)c3)c2)c1C]

Intermediate 118a was synthesized from 117a (1.71 mmol) and 1-methylpyrazole-4-boronic acid pinacol ester (2.05 mmol) as a colorless oil in quantitative yield according to the general method D2.

ESI-MS: 338.15 (M+H)⁺.

The following table illustrates intermediates 118 prepared from method D2:

| | | Synthesis |
|---|---|---|
| Intermediate | Structure | procedure |
| Compound 118a | 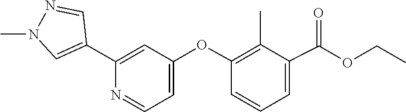 [SMILES: CCOC(=O)c1cccc(Oc2ccnc(-c3cnn(C)c3)c2)c1C] | Method D2 |
| Compound 118b | 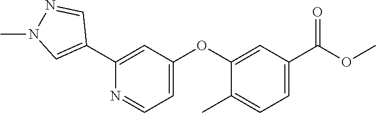 | Method D2 |
| Compound 118c | 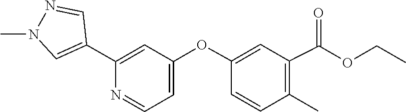 [SMILES: CCOC(=O)c1cc(Oc2ccnc(-c3cnn(C)c3)c2)ccc1C] | Method D2 |
| Compound 118d | 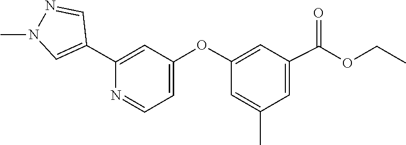 [SMILES: CCOC(=O)c1cc(C)cc(Oc2ccnc(-c3cnn(C)c3)c2)c1] | Method D2 |
| Compound 118h | 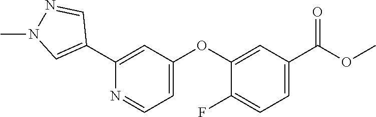 | Method D2 |
| Compound 118i | 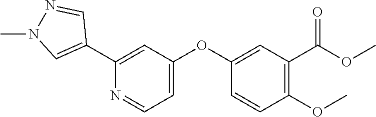 | Method D2 |

Method H: To a solution of 117 (1 equiv.) in dioxane (10 mL/mmol) under nitrogen were added amine derivative (2 equiv.), Pd₂dba₃ (0.1 equiv.), Xantphos (0.2 equiv.) and Cs₂CO₃ (2 equiv.). The mixture was stirred at 100° C. until completion (from 2 h to overnight). The reaction mixture was concentrated under reduced pressure. The crude was purified by flash column chromatography (DCM/MeOH from 100/0 to 90/10) to give the expected compound.

The following compound 118e is an example illustrating Method H:

### Preparation of ethyl 2-methyl-3-({2-[(1-methylpyrazol-3-yl)amino]-4-pyridyl}oxy)benzoate (118e)

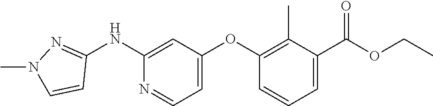 [SMILES: CCOC(=O)c1cccc(Oc2ccnc(Nc3ccn(C)n3)c2)c1C]

Intermediate 118e was synthesized from 117a (1.37 mmol) and 1-methylpyrazol-3-amine (2.74 mmol) as a yellow oil in 90% yield according to the general method H.

ESI-MS: 353.05 (M+H)⁺.

The following table illustrates intermediates 113 prepared from method H:

| | | Synthesis |
|---|---|---|
| Intermediate | Structure | procedure |
| Compound 118e | 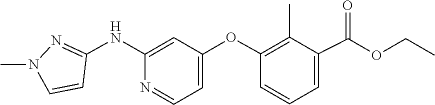 [SMILES: CCOC(=O)c1cccc(Oc2ccnc(Nc3ccn(C)n3)c2)c1C] | Method H |
| Compound 118f | 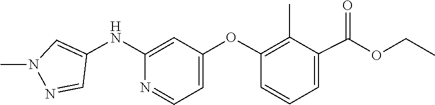 [SMILES: CCOC(=O)c1cccc(Oc2ccnc(Nc3cnn(C)c3)c2)c1C] | Method H |
| Compound 118g | 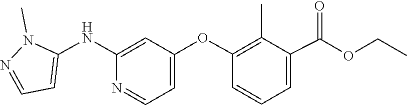 [SMILES: CCOC(=O)c1cccc(Oc2ccnc(Nc3ccnn3C)c2)c1C] | Method H |
| Compound 118j | 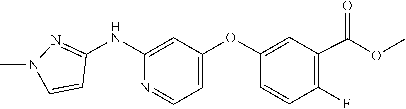 | Method H |
| Compound 118k | 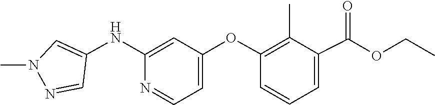 [SMILES: CCOC(=O)c1cccc(Oc2ccnc(Nc3cnn(C)c3)c2)c1C] | Method H |

Method I: To a stirred solution of 117a (250 mg, 0.86 mmol) in CH₃CN (6 mL) were added pyrazole (123 mg, 1.79 mmol), Cs₂CO₃ (1.12 g, 3.42 mmol), CuI (360 mg, 1.88 mmol) and DMEDA (0.323 mL, 3 mmol). The reaction mixture was stirred at 100° C. for 48 h. The reaction mixture was concentrated under reduced pressure. The crude was purified by flash column chromatography (Cyclohexane/EtOAc from 100/0 to 75/25) to give 58 mg of ethyl 2-methyl-3-[(2-pyrazol-1-yl-4-pyridyl)oxy]benzoate 118 h in 11% yield.

The following compound 119a is an example illustrating Method B2:

### Preparation of 2-methyl-3-{[2-(1-methylpyrazol-4-yl)-4-pyridyl]oxy}benzoic acid (119a)

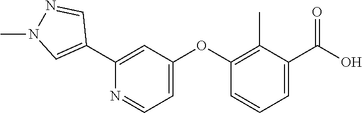 [SMILES: Cc1c(Oc2ccnc(-c3cnn(C)c3)c2)cccc1C(=O)O]

Intermediate 119a was synthesized from 118a (1.88 mmol) as a white powder in 79% yield according to the general method B2.

ESI-MS: 292.00 (M+H)⁺.

The following table illustrates intermediates 119 prepared from method B2:

| | | Synthesis |
|---|---|---|
| Intermediate | Structure | procedure |
| Compound 119a | 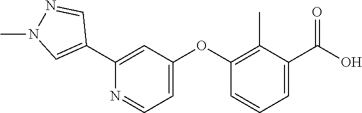 [SMILES: Cc1c(Oc2ccnc(-c3cnn(C)c3)c2)cccc1C(=O)O] | Method B2 |
| Compound 119b | 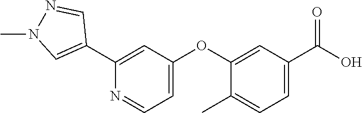 [SMILES: Cc1ccc(C(=O)O)cc1Oc1ccnc(-c2cnn(C)c2)c1] | Method B2 |
| Compound 119c | 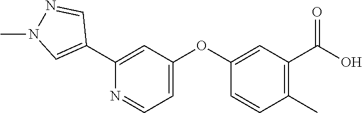 [SMILES: Cc1ccc(Oc2ccnc(-c3cnn(C)c3)c2)cc1C(=O)O] | Method B2 |
| Compound 119d | 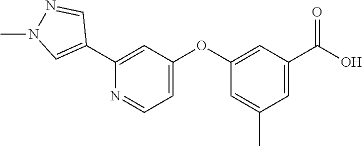 [SMILES: Cc1cc(Oc2ccnc(-c3cnn(C)c3)c2)cc(C(=O)O)c1] | Method B2 |
| Compound 119e | 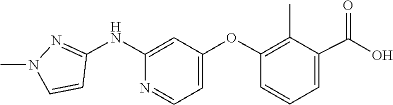 [SMILES: Cc1c(Oc2ccnc(Nc3ccn(C)n3)c2)cccc1C(=O)O] | Method B2 |
| Compound 119f | 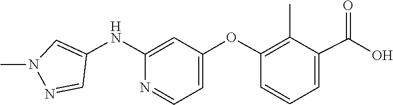 [SMILES: Cc1c(Oc2ccnc(Nc3cnn(C)c3)c2)cccc1C(=O)O] | Method B2 |
| Compound 119g | 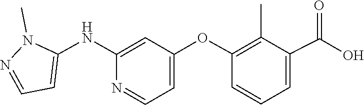 [SMILES: Cc1c(Oc2ccnc(Nc3ccnn3C)c2)cccc1C(=O)O] | Method B2 |
| Compound 119h | 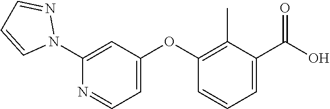 [SMILES: Cc1c(Oc2ccnc(-n3cccn3)c2)cccc1C(=O)O] | Method B2 |
| Compound 119i | 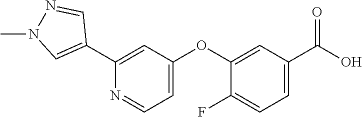 [SMILES: Cn1cc(-c2cc(Oc3cc(C(=O)O)ccc3F)ccn2)cn1] | Method B2 |
| Compound 119j | 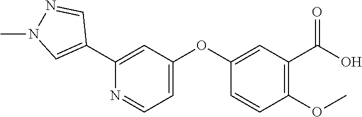 | Method B2 |
| Compound 119k | 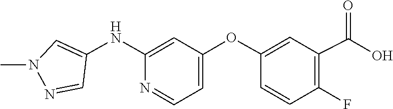 [SMILES: Cn1cc(Nc2cc(Oc3ccc(F)c(C(=O)O)c3)ccn2)cn1] | Method B2 |
| Compound 119l | 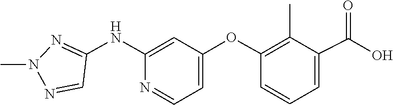 [SMILES: Cc1c(Oc2ccnc(Nc3cnn(C)n3)c2)cccc1C(=O)O] | Method B2 |

The following compounds are examples illustrating Method C2:

### N-[(6-methoxy-3-pyridyl)methyl]-2-methyl-3-{[2-(1-methylpyrazol-4-yl)-4-pyridyl]oxy}benzamide (120)

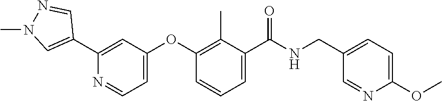

Compound 120 was synthesized from intermediate 119a (0.10 mmol) and (6-methoxypyridin-3-yl) methanamine (0.12 mmol) as a white solid in 60% yield according to the general method C2. ¹H NMR (400 MHz, DMSO-d₆) δ (ppm): 8.91 (t, J=5.9 Hz, 1H), 8.35 (d, J=5.7 Hz, 1H), 8.25 (s, 1H), 8.14 (d, J=2.2 Hz, 1H), 7.96 (s, 1H), 7.69 (dd, J=8.5 Hz, 2.4 Hz, 1H), 7.39-7.28 (m, 2H), 7.24 (d, J=2.3 Hz, 1H), 7.20 (dd, J=7.9 Hz, 1.0 Hz, 1H), 6.81 (d, J=8.5 Hz, 1H), 6.47 (dd, J=5.7 Hz, 2.4 Hz, 1H), 4.39 (d, J=5.9 Hz, 2H), 3.86 (s, 3H), 3.83 (s, 3H), 2.11 (s, 3H).

ESI-MS: 430.10 (M+H)⁺.

### 2-methyl-3-{[2-(1-methylpyrazol-4-yl)-4-pyridyl]oxy}-N-(4-pyridylmethyl)benzamide (121)

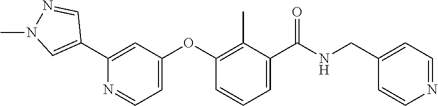 [SMILES: Cc1c(Oc2ccnc(-c3cnn(C)c3)c2)cccc1C(=O)NCc1ccncc1]

Compound 121 was synthesized from intermediate 119a (0.10 mmol) and 4-(aminomethyl)pyridine (0.12 mmol) as a white solid in 69% yield according to the general method C2. ¹H NMR (400 MHz, DMSO-d₆) δ (ppm): 9.04 (t, J=6.0 Hz, 1H), 8.54-8.52 (m, 2H), 8.36 (d, J=5.7 Hz, 1H), 8.25 (s, 1H), 7.96 (s, 1H), 7.43-7.32 (m, 4H), 7.25-7.22 (m, 2H), 6.49 (dd, J=5.7 Hz, 2.4 Hz, 1H), 4.48 (d, J=6.0 Hz, 2H), 3.86 (s, 3H), 2.15 (s, 3H).

ESI-MS: 400.05 (M+H)⁺.

### 2-methyl-3-{[2-(1-methylpyrazol-4-yl)-4-pyridyl]oxy}-N-(3-pyridylmethyl)benzamide (122)

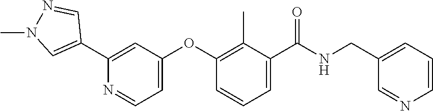 [SMILES: Cc1c(Oc2ccnc(-c3cnn(C)c3)c2)cccc1C(=O)NCc1cccnc1]

Compound 122 was synthesized from intermediate 119a (0.10 mmol) and 3-(aminomethyl)pyridine (0.12 mmol) as a white solid in 59% yield according to the general method C2. ¹H NMR (400 MHz, DMSO-d₆) δ (ppm): 9.00 (t, J=5.9 Hz, 1H), 8.57 (d, J=1.7 Hz, 1H), 8.48 (dd, J=4.7 Hz, 1.4 Hz, 1H), 8.35 (d, J=5.7 Hz, 1H), 8.25 (s, 1H), 7.96 (s, 1H), 7.76 (dt, J=7.8 Hz, 1.8 Hz, 1H) 7.43-7.30 (m, 3H), 7.27-7.20 (m, 2H), 6.47 (dd, J=5.7 Hz, 2.4 Hz, 1H), 4.48 (d, J=5.9 Hz, 2H), 3.86 (s, 3H), 2.12 (s, 3H).

ESI-MS: 400.10 (M+H)⁺.

### N-[(3,5-difluorophenyl)methyl]-2-methyl-3-{[2-(1-methylpyrazol-4-yl)-4-pyridyl]oxy}benzamide (123)

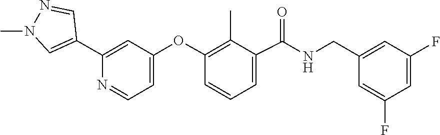 [SMILES: Cc1c(Oc2ccnc(-c3cnn(C)c3)c2)cccc1C(=O)NCc1cc(F)cc(F)c1]

Compound 123 was synthesized from intermediate 119a (0.10 mmol) and 3,5-difluorobenzylamine (0.12 mmol) as a white solid in 85% yield according to the general method C2. ¹H NMR (400 MHz, DMSO-d₆) δ (ppm): 9.01 (t, J=6.0 Hz, 1H), 8.36 (d, J=5.7 Hz, 1H), 8.25 (s, 1H), 7.96 (s, 1H), 7.42-7.34 (m, 2H), 7.26-7.22 (m, 2H), 7.17-7.03 (m, 3H), 6.48 (dd, J=5.7 Hz, 2.4 Hz, 1H), 4.48 (d, J=6.0 Hz, 2H), 3.86 (s, 3H), 2.14 (s, 3H).

ESI-MS: 435.15 (M+H)⁺.

### N-[(3-fluorophenyl)methyl]-2-methyl-3-{[2-(1-methylpyrazol-4-yl)-4-pyridyl]oxy} benzamide (124)

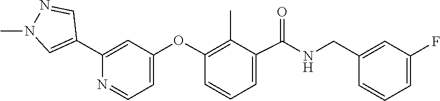 [SMILES: Cc1c(Oc2ccnc(-c3cnn(C)c3)c2)cccc1C(=O)NCc1cccc(F)c1]

Compound 124 was synthesized from intermediate 119a (0.10 mmol) and 3-fluorobenzylamine (0.12 mmol) as a white solid in 82% yield according to the general method C2. ¹H NMR (400 MHz, DMSO-d₆) δ (ppm): 8.98 (t, J=6.0 Hz, 1H), 8.35 (d, J=5.7 Hz, 1H), 8.25 (s, 1H), 7.96 (d, J=0.5 Hz, 1H), 7.43-7.31 (m, 3H), 7.26-7.13 (m, 4H), 7.09 (td, J=8.4 Hz, 2.3 Hz, 1H), 6.48 (dd, J=5.7 Hz, 2.4 Hz, 1H), 4.47 (d, J=6.0 Hz, 2H), 3.86 (s, 3H), 2.14 (s, 3H).

ESI-MS: 417.20 (M+H)⁺.

### N-[(4-fluorophenyl)methyl]-2-methyl-3-{[2-(1-methylpyrazol-4-yl)-4-pyridyl]oxy} benzamide (125)

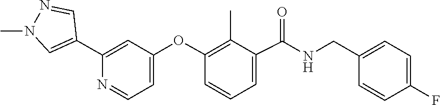 [SMILES: Cc1c(Oc2ccnc(-c3cnn(C)c3)c2)cccc1C(=O)NCc1ccc(F)cc1]

Compound 125 was synthesized from intermediate 119a (0.10 mmol) and 4-fluorobenzylamine (0.12 mmol) as a white solid in 92% yield according to the general method C2. ¹H NMR (400 MHz, DMSO-d₆) δ (ppm): 8.94 (t, J=6.0 Hz, 1H), 8.35 (d, J=5.7 Hz, 1H), 8.25 (s, 1H), 7.96 (d, J=0.5 Hz, 1H), 7.43-7.30 (m, 4H), 7.26-7.14 (m, 4H), 6.47 (dd, J=5.7 Hz, 2.4 Hz, 1H), 4.43 (d, J=6.0 Hz, 2H), 3.86 (s, 3H), 2.12 (s, 3H).

ESI-MS: 417.15 (M+H)⁺.

### N-[(3-chlorophenyl)methyl]-2-methyl-3-{[2-(1-methylpyrazol-4-yl)-4-pyridyl]oxy} benzamide (126)

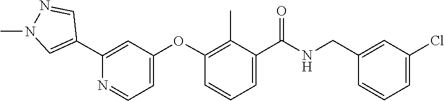 [SMILES: Cc1c(Oc2ccnc(-c3cnn(C)c3)c2)cccc1C(=O)NCc1cccc(Cl)c1]

Compound 126 was synthesized from intermediate 119a (0.10 mmol) and 3-chlorobenzylamine (0.12 mmol) as a white solid in 72% yield according to the general method C2. ¹H NMR (400 MHz, DMSO-d₆) δ (ppm): 8.98 (t, J=6.0 Hz, 1H), 8.35 (d, J=5.7 Hz, 1H), 8.25 (s, 1H), 7.96 (s, 1H), 7.44-7.30 (m, 6H), 7.25 (d, J=2.4 Hz, 1H), 7.22 (dd, J=7.8 Hz, 1.2 Hz, 1H), 6.48 (dd, J=5.7 Hz, 2.4 Hz, 1H), 4.46 (d, J=6.0 Hz, 2H), 3.86 (s, 3H), 2.13 (s, 3H).

ESI-MS: 433.15 (M+H)⁺.

### N-[(4-chlorophenyl)methyl]-2-methyl-3-{[2-(1-methylpyrazol-4-yl)-4-pyridyl]oxy} benzamide (127)

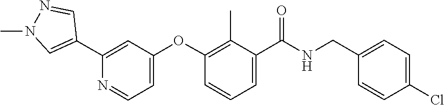 [SMILES: Cc1c(Oc2ccnc(-c3cnn(C)c3)c2)cccc1C(=O)NCc1ccc(Cl)cc1]

Compound 127 was synthesized from intermediate 119a (0.10 mmol) and 4-chlorobenzylamine (0.12 mmol) as a white solid in 83% yield according to the general method C2. ¹H NMR (400 MHz, DMSO-d₆) δ (ppm): 8.97 (t, J=6.0 Hz, 1H), 8.35 (d, J=5.7 Hz, 1H), 8.25 (s, 1H), 7.96 (s, 1H), 7.45-7.30 (m, 6H), 7.25-7.20 (m, 2H), 6.48 (dd, J=5.7 Hz, 2.4 Hz, 1H), 4.44 (d, J=6.0 Hz, 2H), 3.86 (s, 3H), 2.13 (s, 3H).

ESI-MS: 433.05 (M+H)⁺.

### N-[(2,4-difluorophenyl)methyl]-2-methyl-3-{[2-(1-methylpyrazol-4-yl)-4-pyridyl]oxy}benzamide (128)

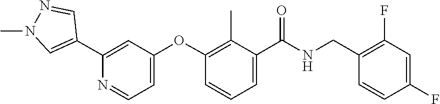 [SMILES: Cc1c(Oc2ccnc(-c3cnn(C)c3)c2)cccc1C(=O)NCc1ccc(F)cc1F]

Compound 128 was synthesized from intermediate 119a (0.065 mmol) and 2,4-difluorobenzylamine (0.097 mmol) as a white solid in 53% yield according to the general method C2. ¹H NMR (400 MHz, DMSO-d₆) δ (ppm): 8.93 (t, J=5.6 Hz, 1H), 8.35 (d, J=5.7 Hz, 1H), 8.25 (s, 1H), 7.96 (s, 1H), 7.51-7.43 (m, 1H), 7.40-7.18 (m, 5H), 7.09 (dd, J=8.5 Hz, 7.0 Hz, 1H), 6.47 (dd, J=5.6 Hz, 2.2 Hz, 1H), 4.46 (d, J=5.5 Hz, 2H), 3.86 (s, 3H), 2.11 (s, 3H).

ESI-MS: 435.15 (M+H)⁺.

### N-[(3,4-difluorophenyl)methyl]-2-methyl-3-{[2-(1-methylpyrazol-4-yl)-4-pyridyl] oxy}benzamide (129)

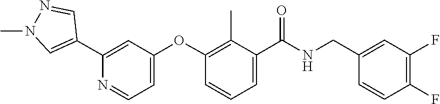 [SMILES: Cc1c(Oc2ccnc(-c3cnn(C)c3)c2)cccc1C(=O)NCc1ccc(F)c(F)c1]

Compound 129 was synthesized from intermediate 119a (0.065 mmol) and 3,4-difluorobenzylamine (0.097 mmol) as a white solid in 57% yield according to the general method C2. ¹H NMR (400 MHz, DMSO-d₆) δ (ppm): 8.98 (t, J=6.0 Hz, 1H), 8.35 (d, J=5.7 Hz, 1H), 8.25 (s, 1H), 7.96 (s, 1H), 7.47-7.30 (m, 4H), 7.27-7.17 (m, 3H), 6.48 (dd, J=5.7 Hz, 2.4 Hz, 1H), 4.44 (d, J=6.0 Hz, 2H), 3.86 (s, 3H), 2.13 (s, 3H).

ESI-MS: 435.15 (M+H)⁺.

### N-[(4-chloro-3-fluoro-phenyl)methyl]-2-methyl-3-{[2-(1-methylpyrazol-4-yl)-4-pyridyl]oxy}benzamide (130)

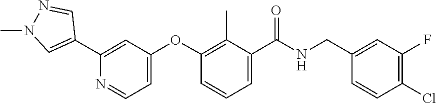 [SMILES: Cc1c(Oc2ccnc(-c3cnn(C)c3)c2)cccc1C(=O)NCc1ccc(Cl)c(F)c1]

Compound 130 was synthesized from intermediate 119a (0.065 mmol) and 4-chloro-3-fluorobenzylamine (0.097 mmol) as a white solid in 34% yield according to the general method C2. ¹H NMR (400 MHz, DMSO-d₆) δ (ppm): 9.00 (t, J=6.0 Hz, 1H), 8.35 (d, J=5.7 Hz, 1H), 8.25 (s, 1H), 7.96 (s, 1H), 7.58 (t, J=8.0 Hz, 1H), 7.43-7.32 (m, 3H), 7.28-7.18 (m, 3H), 6.48 (dd, J=5.7 Hz, 2.4 Hz, 1H), 4.46 (d, J=6.0 Hz, 2H), 3.86 (s, 3H), 2.13 (s, 3H).

ESI-MS: 451.10 (M+H)⁺.

### N-(imidazo[1,2-a]pyridin-6-ylmethyl)-2-methyl-3-{[2-(1-methylpyrazol-4-yl)-4-pyridyl]oxy}benzamide (131)

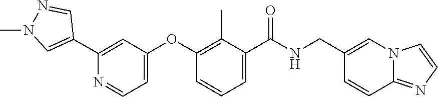 [SMILES: Cc1c(Oc2ccnc(-c3cnn(C)c3)c2)cccc1C(=O)NCc1ccc2nccn2c1]

Compound 131 was synthesized from intermediate 119a (0.065 mmol) and imidazo[1,2-a]pyridin-6-ylmethanamine (0.097 mmol) as a white solid in 57% yield according to the general method C2. ¹H NMR (400 MHz, DMSO-d₆) δ (ppm): 8.97 (t, J=5.9 Hz, 1H), 8.51 (s, 1H), 8.35 (d, J=5.7 Hz, 1H), 8.25 (s, 1H), 7.97-7.95 (m, 2H), 7.57-5.55 (m, 2H), 7.41-7.32 (m, 2H), 7.26-7.20 (m, 3H), 6.47 (dd, J=5.7 Hz, 2.4 Hz, 1H), 4.45 (d, J=5.8 Hz, 2H), 3.86 (s, 3H), 2.13 (s, 3H).

ESI-MS: 439.15 (M+H)⁺.

### N-(imidazo[1,2-a]pyridin-7-ylmethyl)-2-methyl-3-{[2-(1-methylpyrazol-4-yl)-4-pyridyl]oxy}benzamide (132)

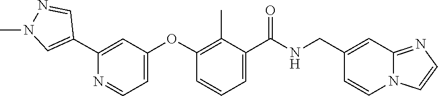 [SMILES: Cc1c(Oc2ccnc(-c3cnn(C)c3)c2)cccc1C(=O)NCc1ccn2ccnc2c1]

Compound 132 was synthesized from intermediate 119a (0.065 mmol) and imidazo[1,2-a]pyridin-7-ylmethanamine (0.097 mmol) as a white solid in 50% yield according to the general method C2. ¹H NMR (400 MHz, DMSO-d₆) δ (ppm): 9.01 (t, J=6.0 Hz, 1H), 8.51 (d, J=7.0 Hz, 1H), 8.36 (d, J=5.7 Hz, 1H), 8.25 (s, 1H), 7.96 (s, 1H), 7.90 (s, 1H), 7.53 (d, J=1.0 Hz, 1H), 7.44 (s, 1H), 7.41-7.34 (m, 2H), 7.28-7.20 (m, 2H), 6.89 (dd, J=7.0 Hz, Hz 1.5, 1H), 6.49 (dd, J=5.7 Hz, 2.4 Hz, 1H), 4.49 (d, J=5.9 Hz, 2H), 3.86 (s, 3H), 2.15 (s, 3H).

ESI-MS: 439.15 (M+H)⁺.

### 2-methyl-3-{[2-(1-methylpyrazol-4-yl)-4-pyridyl]oxy}-N-{[6-(trifluoromethyl)-3-pyridyl]methyl}benzamide (133)

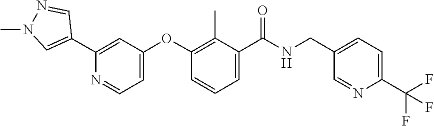 [SMILES: Cc1c(Oc2ccnc(-c3cnn(C)c3)c2)cccc1C(=O)NCc1ccc(C(F)(F)F)nc1]

Compound 133 was synthesized from intermediate 119a (0.065 mmol) and [6-(trifluoromethyl)-3-pyridyl]methanamine (0.097 mmol) as a white solid in 79% yield according to the general method C2. ¹H NMR (400 MHz, DMSO-d₆) δ (ppm): 9.09 (t, J=5.9 Hz, 1H), 8.76 (s, 1H), 8.35 (d, J=5.7 Hz, 1H), 8.25 (s, 1H), 8.08-8.02 (m, 1H), 7.96 (s, 1H), 7.92 (d, J=8.1 Hz, 1H), 7.43-7.35 (m, 2H), 7.27-7.20 (m, 2H), 6.48 (dd, J=5.7 Hz, 2.4 Hz, 1H), 4.59 (d, J=5.8 Hz, 2H), 3.86 (s, 3H), 2.14 (s, 3H).

ESI-MS: 468.15 (M+H)⁺.

### N-[(2,3-difluorophenyl)methyl]-2-methyl-3-{[2-(1-methylpyrazol-4-yl)-4-pyridyl] oxy}benzamide (134)

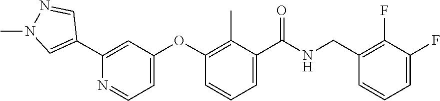 [SMILES: Cc1c(Oc2ccnc(-c3cnn(C)c3)c2)cccc1C(=O)NCc1cccc(F)c1F]

Compound 134 was synthesized from intermediate 119a (0.074 mmol) and 2,3-difluorobenzylamine (0.111 mmol) as a white solid in 60% yield according to the general method C2. ¹H NMR (400 MHz, DMSO-d₆) δ (ppm): 8.97 (t, J=5.8 Hz, 1H), 8.35 (d, J=5.7 Hz, 1H), 8.24 (s, 1H), 7.95 (d, J=0.6 Hz, 1H), 7.40-7.29 (m, 3H), 7.28-7.18 (m, 4H), 6.48 (dd, J=5.7 Hz, 2.4 Hz, 1H), 4.53 (d, J=5.8 Hz, 2H), 3.86 (s, 3H), 2.13 (s, 3H).

ESI-MS: 435.15 (M+H)⁺.

### N-[(3-methoxyphenyl)methyl]-2-methyl-3-{[2-(1-methylpyrazol-4-yl)-4-pyridyl] oxy}benzamide (135)

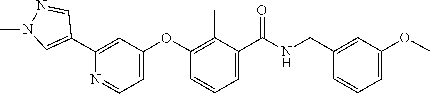

Compound 135 was synthesized from intermediate 119a (0.074 mmol) and 3-methoxybenzylamine (0.111 mmol) as a white solid in 78% yield according to the general method C2. ¹H NMR (400 MHz, DMSO-d₆) δ (ppm): 8.90 (t, J=6.0 Hz, 1H), 8.35 (d, J=5.7 Hz, 1H), 8.24 (s, 1H), 7.95 (d, J=0.6 Hz, 1H), 7.43-7.16 (m, 5H), 6.98-6.90 (m, 2H), 6.86-6.80 (m, 1H), 6.49 (dd, J=5.7 Hz, 2.4 Hz, 1H), 4.44 (d, J=6.0 Hz, 2H), 3.86 (s, 3H), 3.74 (s, 3H), 2.14 (s, 3H).

ESI-MS: 429.20 (M+H)⁺.

### N-[(4-methoxyphenyl)methyl]-2-methyl-3-{[2-(1-methylpyrazol-4-yl)-4-pyridyl] oxy}benzamide (136)

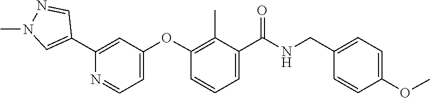

Compound 136 was synthesized from intermediate 119a (0.074 mmol) and 4-methoxybenzylamine (0.111 mmol) as a white solid in 63% yield according to the general method C2. ¹H NMR (400 MHz, DMSO-d₆) δ (ppm): 8.83 (t, J=6.0 Hz, 1H), 8.35 (d, J=5.7 Hz, 1H), 8.24 (s, 1H), 7.95 (d, J=0.6 Hz, 1H), 7.36 (t, J=7.8 Hz, 1H), 7.30-7.18 (m, 5H), 6.92-6.88 (m, 2H), 6.48 (dd, J=5.7 Hz, 2.4 Hz, 1H), 4.38 (d, J=6.0 Hz, 2H), 3.86 (s, 3H), 3.73 (s, 3H), 2.12 (s, 3H).

ESI-MS: 429.15 (M+H)⁺.

### N-[(5-fluoro-3-pyridyl)methyl]-2-methyl-3-{[2-(1-methylpyrazol-4-yl)-4-pyridyl] oxy}benzamide (137)

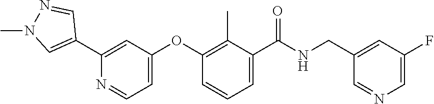 [SMILES: Cc1c(Oc2ccnc(-c3cnn(C)c3)c2)cccc1C(=O)NCc1cncc(F)c1]

Compound 137 was synthesized from intermediate 119a (0.10 mmol) and 5-fluoro-3-pyridinemethanamine (0.15 mmol) as a white solid in 50% yield according to the general method C2. ¹H NMR (400 MHz, DMSO-d₆) δ (ppm): 9.01 (t, J=5.9 Hz, 1H), 8.52-8.45 (m, 2H), 8.36 (d, J=5.7 Hz, 1H), 8.24 (s, 1H), 7.95 (d, J=0.6 Hz, 1H), 7.71-7.64 (m, 1H), 7.41-7.33 (m, 2H), 7.27-7.20 (m, 2H), 6.48 (dd, J=5.7 Hz, 2.4 Hz, 1H), 4.53 (d, J=5.9 Hz, 2H), 3.86 (s, 3H), 2.13 (s, 3H).

ESI-MS: 418.15 (M+H)⁺.

### N-[(5-fluoro-6-methoxy-3-pyridyl)methyl]-2-methyl-3-{[2-(1-methylpyrazol-4-yl)-4-pyridyl]oxy}benzamide (138)

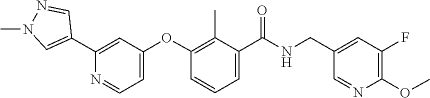

Compound 138 was synthesized from intermediate 119a (0.10 mmol) and (5-fluoro-6-methoxy-3-pyridyl) methanamine (0.15 mmol) as a white solid in 62% yield according to the general method C2. ¹H NMR (400 MHz, DMSO-d₆) δ (ppm): 8.91 (t, J=5.9 Hz, 1H), 8.35 (d, J=5.7 Hz, 1H), 8.24 (s, 1H), 7.98-7.95 (m, 2H), 7.65 (dd, J=11.4 Hz, 1.9 Hz, 1H), 7.42-7.31 (m, 2H), 7.26-7.19 (m, 2H), 6.48 (dd, J=5.7 Hz, 2.4 Hz, 1H), 4.41 (d, J=5.9 Hz, 2H), 3.93 (s, 3H), 3.86 (s, 3H), 2.12 (s, 3H).

ESI-MS: 448.15 (M+H)⁺.

### N-[(5-fluoro-2-methoxy-3-pyridyl)methyl]-2-methyl-3-{[2-(1-methylpyrazol-4-yl)-4-pyridyl]oxy}benzamide (139)

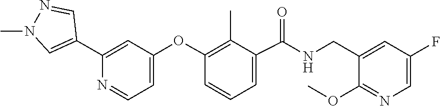 [SMILES: COc1ncc(F)cc1CNC(=O)c1cccc(Oc2ccnc(-c3cnn(C)c3)c2)c1C]

Compound 139 was synthesized from intermediate 119a (0.10 mmol) and 5-fluoro-2-methoxy-3-pyridinemethanamine (0.24 mmol) as a white solid in 40% yield according to the general method C2. ¹H NMR (400 MHz, DMSO-d₆) δ (ppm): 8.85 (t, J=5.7 Hz, 1H), 8.36 (d, J=5.7 Hz, 1H), 8.24 (s, 1H), 8.07 (d, J=3.0 Hz, 1H), 7.96 (d, J=0.6 Hz, 1H), 7.57 (dd, J=8.6 Hz, 3.0 Hz, 1H), 7.41-7.36 (m, 2H), 7.24-7.21 (m, 2H), 6.49 (dd, J=5.7 Hz, 2.4 Hz, 1H), 4.39 (d, J=5.7 Hz, 2H), 3.91 (s, 3H), 3.86 (s, 3H), 2.15 (s, 3H).

ESI-MS: 448.20 (M+H)⁺.

### N-[(3-fluoro-4-methoxy-phenyl)methyl]-2-methyl-3-{[2-(1-methylpyrazol-4-yl)-4-pyridyl]oxy}benzamide (140)

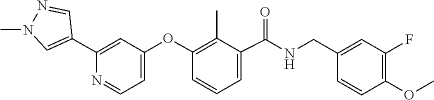

Compound 140 was synthesized from intermediate 119a (0.10 mmol) and (3-fluoro-4-methoxyphenyl) methanamine (0.15 mmol) as a white solid in 58% yield according to the general method C2. ¹H NMR (400 MHz, DMSO-d₆) δ (ppm): 8.88 (t, J=6.0 Hz, 1H), 8.35 (d, J=5.7 Hz, 1H), 8.24 (s, 1H), 7.95 (d, J=0.6 Hz, 1H), 7.37 (t, J=7.8 Hz, 1H), 7.31 (dd, J=7.6 Hz, 1.3 Hz, 1H), 7.24-7.09 (m, 5H), 6.48 (dd, J=5.7 Hz, 2.4 Hz, 1H), 4.39 (d, J=6.0 Hz, 2H), 3.86 (s, 3H), 3.82 (s, 3H), 2.13 (s, 3H).

ESI-MS: 447.20 (M+H)⁺.

### N-[(4-fluoro-3-methoxy-phenyl)methyl]-2-methyl-3-{[2-(1-methylpyrazol-4-yl)-4-pyridyl]oxy}benzamide (141)

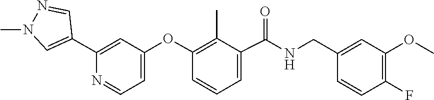

Compound 141 was synthesized from intermediate 119a (0.10 mmol) and 5-(aminomethyl)-2-fluoroanisole (0.15 mmol) as a white solid in 70% yield according to the general method C2. ¹H NMR (400 MHz, DMSO-d₆) δ (ppm): 8.90 (t, J=6.0 Hz, 1H), 8.35 (d, J=5.7 Hz, 1H), 8.24 (s, 1H), 7.95 (d, J=0.6 Hz, 1H), 7.42-7.30 (m, 2H), 7.25-7.12 (m, 4H), 6.92-6.88 (m, 1H), 6.49 (dd, J=5.7 Hz, 2.4 Hz, 1H), 4.43 (d, J=6.0 Hz, 2H), 3.86 (s, 3H), 3.82 (s, 3H), 2.14 (s, 3H).

ESI-MS: 447.20 (M+H)⁺.

### N-(cyclohexylmethyl)-2-methyl-3-{[2-(1-methylpyrazol-4-yl)-4-pyridyl]oxy} benzamide (142)

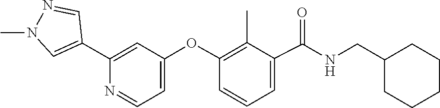 [SMILES: Cc1c(Oc2ccnc(-c3cnn(C)c3)c2)cccc1C(=O)NCC1CCCCC1]

Compound 142 was synthesized from intermediate 119a (0.10 mmol) and 1-cyclohexylmethanamine (0.15 mmol) as a white solid in 69% yield according to the general method C2. ¹H NMR (400 MHz, DMSO-d₆) δ (ppm): 8.36-3.81 (m, 2H), 8.24 (s, 1H), 7.96 (s, 1H), 7.35 (t, J=7.8 Hz, 1H), 7.27-7.22 (m, 2H), 7.18 (d, J=8.0 Hz, 1H), 6.48 (dd, J=5.7 Hz, 2.4 Hz, 1H), 3.87 (s, 3H), 3.09 (t, J=6.4 Hz, 2H), 2.13 (s, 3H), 1.77-1.47 (m, 6H), 1.27-1.12 (m, 3H), 0.99-0.89 (m, 2H).

ESI-MS: 405.20 (M+H)⁺.

### 2-methyl-3-{[2-(1-methylpyrazol-4-yl)-4-pyridyl]oxy}-N-(tetrahydropyran-4-ylmethyl)benzamide (143)

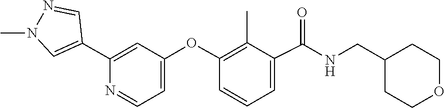 [SMILES: Cc1c(Oc2ccnc(-c3cnn(C)c3)c2)cccc1C(=O)NCC1CCOCC1]

Compound 143 was synthesized from intermediate 119a (0.10 mmol) and 4-(aminomethyl)tetrahydropyran (0.15 mmol) as a white solid in 71% yield according to the general method C2. ¹H NMR (400 MHz, DMSO-d₆) δ (ppm): 8.39 (t, J=5.8 Hz, 1H), 8.36 (d, J=5.7 Hz, 1H), 8.24 (s, 1H), 7.96 (d, J=0.6 Hz, 1H), 7.35 (t, J=7.7 Hz, 1H), 7.28-7.21 (m, 2H), 7.19 (dd, J=8.0 Hz, 1.0 Hz, 1H), 6.48 (dd, J=5.7 Hz, 2.4 Hz, 1H), 3.89-3.82 (m, 5H), 3.27-3.24 (m, 2H), 3.18-3.12 (m, 2H), 2.13 (s, 3H), 1.80-1.74 (m, 1H), 1.63-1.60 (m, 2H), 1.26-1.16 (m, 2H).

ESI-MS: 407.15 (M+H)⁺.

### N-[(1R)-1-cyclohexylethyl]-2-methyl-3-{[2-(1-methylpyrazol-4-yl)-4-pyridyl]oxy} benzamide (144)

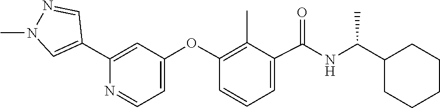 [SMILES: Cc1c(Oc2ccnc(-c3cnn(C)c3)c2)cccc1C(=O)N[C@H](C)C1CCCCC1]

Compound 144 was synthesized from intermediate 119a (0.10 mmol) and (R)-1-cyclohexylethanamine (0.15 mmol) as a white solid in 60% yield according to the general method C2. ¹H NMR (400 MHz, DMSO-d₆) δ (ppm): 8.36 (d, J=5.7 Hz, 1H), 8.25 (s, 1H), 8.15 (d, J=8.7 Hz, 1H), 7.96 (d, J=0.5 Hz, 1H), 7.35 (t, J=7.8 Hz, 1H), 7.27-7.14 (m, 3H), 6.48 (dd, J=5.7 Hz, 2.4 Hz, 1H), 3.92-3.76 (m, 4H), 2.12 (s, 3H), 1.83-1.57 (m, 5H), 1.38-1.33 (m, 1H), 1.24-0.92 (m, 8H).

ESI-MS: 419.20 (M+H)⁺.

### N-[(1S)-1-cyclohexylethyl]-2-methyl-3-{[2-(1-methylpyrazol-4-yl)-4-pyridyl]oxy} benzamide (145)

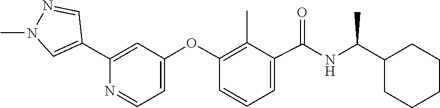 [SMILES: Cc1c(Oc2ccnc(-c3cnn(C)c3)c2)cccc1C(=O)N[C@@H](C)C1CCCCC1]

Compound 145 was synthesized from intermediate 119a (0.10 mmol) and(S)-1-cyclohexylethanamine (0.15 mmol) as a white solid in 54% yield according to the general method C2. ¹H NMR (400 MHz, DMSO-d₆) δ (ppm): 8.36 (d, J=5.7 Hz, 1H), 8.25 (s, 1H), 8.15 (d, J=8.8 Hz, 1H), 7.96 (s, 1H), 7.35 (t, J=7.7 Hz, 1H), 7.26-7.15 (m, 3H), 6.48 (dd, J=5.7 Hz, 2.4 Hz, 1H), 3.90-3.75 (m, 4H), 2.12 (s, 3H), 1.82-1.58 (m, 5H), 1.42-1.33 (m, 1H), 1.24-1.06 (m, 6H), 1.03-0.94 (m, 2H).

ESI-MS: 419.20 (M+H)⁺.

### 2-methyl-N-[(1-methylpyrazol-4-yl)methyl]-3-{[2-(1-methylpyrazol-4-yl)-4-pyridyl]oxy}benzamide (146)

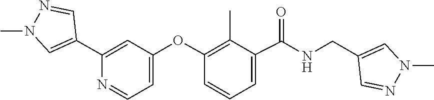

Compound 146 was synthesized from intermediate 119a (0.10 mmol) and C-(1-methyl-1H-pyrazol-4-yl)-methylamine (0.15 mmol) as a white solid in 62% yield according to the general method C2. ¹H NMR (400 MHz, DMSO-d₆) δ (ppm): 8.69 (t, J=5.7 Hz, 1H), 8.35 (d, J=5.7 Hz, 1H), 8.24 (s, 1H), 7.95 (d, J=0.6 Hz, 1H), 7.61 (s, 1H), 7.37-7.32 (m, 2H), 7.27 (dd, J=7.6 Hz, 1.2 Hz, 1H), 7.22 (d, J=2.3 Hz, 1H), 7.18 (dd, J=8.0 Hz, 1.1 Hz, 1H), 6.47 (dd, J=5.7 Hz, 2.4 Hz, 1H), 4.27 (d, J=5.7 Hz, 2H), 3.86 (s, 3H), 3.79 (s, 3H), 2.12 (s, 3H).

ESI-MS: 403.15 (M+H)⁺.

### 2-methyl-N-[(2-methylpyrazol-3-yl)methyl]-3-{[2-(1-methylpyrazol-4-yl)-4-pyridyl]oxy}benzamide (147)

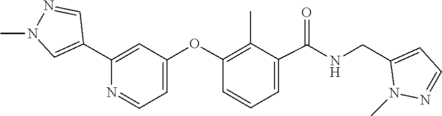 [SMILES: Cc1c(Oc2ccnc(-c3cnn(C)c3)c2)cccc1C(=O)NCc1ccnn1C]

Compound 147 was synthesized from intermediate 119a (0.10 mmol) and C-(2-methyl-2H-pyrazol-3-yl)-methylamine (0.15 mmol) as a white solid in 69% yield according to the general method C2. ¹H NMR (400 MHz, DMSO-d₆) δ (ppm): 8.89 (t, J=5.7 Hz, 1H), 8.35 (d, J=5.7 Hz, 1H), 8.24 (s, 1H), 7.95 (d, J=0.6 Hz, 1H), 7.36 (t, J=7.8 Hz, 1H), 7.32-7.28 (m, 2H), 7.23 (d, J=2.3 Hz, 1H), 7.21 (dd, J=7.9 Hz, 1.2 Hz, 1H), 6.48 (dd, J=5.7 Hz, 2.4 Hz, 1H), 6.19 (d, J=1.8 Hz, 1H), 4.51 (d, J=5.7 Hz, 2H), 3.86 (s, 3H), 3.83 (s, 3H), 2.13 (s, 3H).

ESI-MS: 403.20 (M+H)⁺.

### 2-methyl-N-[(1-methylpyrazol-3-yl)methyl]-3-{[2-(1-methylpyrazol-4-yl)-4-pyridyl]oxy}benzamide (148)

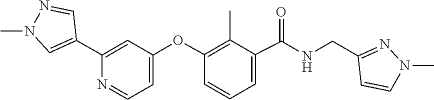

Compound 148 was synthesized from intermediate 119a (0.10 mmol) and (1-methyl-1H-pyrazol-3-yl) methanamine (0.15 mmol) as a white solid in 72% yield according to the general method C2. ¹H NMR (400 MHz, DMSO-d₆) δ (ppm): 8.74 (t, J=5.9 Hz, 1H), 8.35 (d, J=5.7 Hz, 1H), 8.24 (s, 1H), 7.95 (d, J=0.6 Hz, 1H), 7.59 (d, J=2.1 Hz, 1H), 7.35 (t, J=7.7 Hz, 1H), 7.27 (dd, J=7.6 Hz, 1.2 Hz, 1H), 7.22 (d, J=2.3 Hz, 1H), 7.18 (dd, J=7.9 Hz, 1.1 Hz, 1H), 6.49 (dd, J=5.7 Hz, 2.4 Hz, 1H), 6.16 (d, J=2.2 Hz, 1H), 4.38 (d, J=5.9 Hz, 2H), 3.86 (s, 3H), 3.78 (s, 3H), 2.13 (s, 3H).

ESI-MS: 403.15 (M+H)⁺.

### N-[(4-fluoro-3-methyl-phenyl)methyl]-2-methyl-3-{[2-(1-methylpyrazol-4-yl)-4-pyridyl]oxy}benzamide (149)

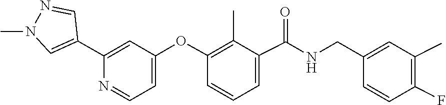 [SMILES: Cc1cc(CNC(=O)c2cccc(Oc3ccnc(-c4cnn(C)c4)c3)c2C)ccc1F]

Compound 149 was synthesized from intermediate 119a (0.10 mmol) and (4-fluoro-3-methylphenyl) methanamine (0.15 mmol) as a white solid in 67% yield according to the general method C2. ¹H NMR (400 MHz, DMSO-d₆) δ (ppm): 8.88 (t, J=6.0 Hz, 1H), 8.35 (d, J=5.7 Hz, 1H), 8.24 (s, 1H), 7.95 (s, 1H), 7.42-7.29 (m, 2H), 7.28-7.16 (m, 4H), 7.13-7.06 (m, 1H), 6.48 (dd, J=5.7 Hz, 2.4 Hz, 1H), 4.40 (d, J=6.0 Hz, 2H), 3.86 (s, 3H), 2.22 (d, J=1.5 Hz, 3H), 2.13 (s, 3H).

ESI-MS: 431.15 (M+H)⁺.

### N-[(3-fluoro-4-methyl-phenyl)methyl]-2-methyl-3-{[2-(1-methylpyrazol-4-yl)-4-pyridyl]oxy}benzamide (150)

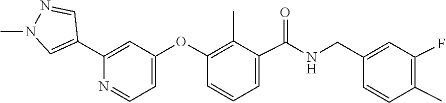

Compound 150 was synthesized from intermediate 119a (0.10 mmol) and 3-fluoro-4-methylbenzylamine (0.15 mmol) as a white solid in 69% yield according to the general method C2. ¹H NMR (400 MHz, DMSO-d₆) δ (ppm): 8.91 (t, J=6.0 Hz, 1H), 8.35 (d, J=5.7 Hz, 1H), 8.24 (s, 1H), 7.95 (d, J=0.6 Hz, 1H), 7.40-7.30 (m, 2H), 7.27-7.17 (m, 3H), 7.13-7.07 (m, 2H), 6.49 (dd, J=5.7 Hz, 2.4 Hz, 1H), 4.42 (d, J=6.0 Hz, 2H), 3.86 (s, 3H), 2.21 (d, J=1.2 Hz, 3H), 2.13 (s, 3H).

ESI-MS: 431.15 (M+H)⁺.

### 2-methyl-3-{[2-(1-methylpyrazol-4-yl)-4-pyridyl]oxy}-N-[(2-oxo-1H-pyridin-3-yl)methyl]benzamide (151)

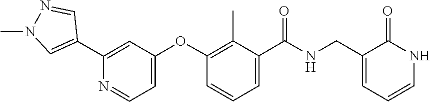 [SMILES: Cc1c(Oc2ccnc(-c3cnn(C)c3)c2)cccc1C(=O)NCc1ccc[nH]c1=O]

Compound 151 was synthesized from intermediate 119a (0.10 mmol) and 3-(aminomethyl)-2 (1H)-pyridinone (0.24 mmol) as a white solid in 40% yield according to the general method C2. ¹H NMR (400 MHz, DMSO-d₆) δ (ppm): 11.62 (bs, 1H), 8.66 (t, J=5.8 Hz, 1H), 8.36 (d, J=5.7 Hz, 1H), 8.24 (s, 1H), 7.96 (d, J=0.6 Hz, 1H), 7.37-7.35 (m, 3H), 7.30 (dd, J=6.5 Hz, 2.0 Hz, 1H), 7.25-7.18 (m, 2H), 6.49 (dd, J=5.7 Hz, 2.4 Hz, 1H), 6.20 (t, J=6.6 Hz, 1H), 4.21 (d, J=5.8 Hz, 2H), 3.86 (s, 3H), 2.15 (s, 3H).

ESI-MS: 416.15 (M+H)⁺.

### 2-methyl-N-[(1-methyl-2-oxo-3-piperidyl)methyl]-3-{[2-(1-methylpyrazol-4-yl)-4-pyridyl]oxy}benzamide (152)

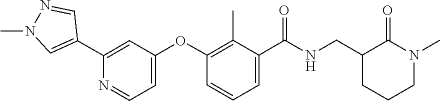

Compound 152 was synthesized from intermediate 119a (0.10 mmol) and 3-(aminomethyl)-1-methyl-2-piperidinone (0.24 mmol) as a white solid in 69% yield according to the general method C2. ¹H NMR (400 MHz, DMSO-d₆) δ (ppm): 8.36 (d, J=5.7 Hz, 1H), 8.30 (t, J=5.8 Hz, 1H), 8.24 (s, 1H), 7.96 (s, 1H), 7.35 (t, J=7.8 Hz, 1H), 7.28 (d, J=6.6 Hz, 1H), 7.23 (d, J=2.4 Hz, 1H), 7.19 (d, J=7.9 Hz, 1H), 6.49 (dd, J=5.7 Hz, 2.4 Hz, 1H), 3.87 (s, 3H), 3.67-3.61 (m, 1H), 3.41-3.33 (m, 2H), 3.27-3.20 (m, 2H), 2.81 (s, 3H), 2.13 (s, 3H), 1.92-1.84 (m, 2H), 1.72-1.54 (m, 2H).

ESI-MS: 434.20 (M+H)⁺.

### 2-methyl-N-[(1-methyl-2-oxo-3-pyridyl)methyl]-3-{[2-(1-methylpyrazol-4-yl)-4-pyridyl]oxy}benzamide (153)

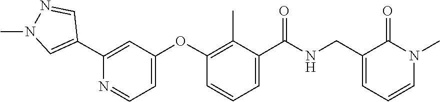

Compound 153 was synthesized from intermediate 119a (0.07 mmol) and 3-(aminomethyl)-1-methyl-2 (1H)-pyridinone (0.15 mmol) as a white solid in 53% yield according to the general method C2. ¹H NMR (400 MHz, DMSO-d₆) δ (ppm): 8.69 (t, J=5.8 Hz, 1H), 8.36 (d, J=5.8 Hz, 1H), 8.25 (s, 1H), 7.96 (d, J=0.7 Hz, 1H), 7.63 (dd, J=6.7 Hz, 1.9 Hz, 1H), 7.41-7.33 (m, 3H), 7.27-7.18 (m, 2H), 6.49 (dd, J=5.7 Hz, 2.4 Hz, 1H), 6.24 (t, J=6.8 Hz, 1H), 4.24 (d, J=5.8 Hz, 2H), 3.86 (s, 3H), 3.46 (s, 3H), 2.15 (s, 3H).

ESI-MS: 430.20 (M+H)⁺.

### N-[(5-fluoro-2-pyridyl)methyl]-2-methyl-3-{[2-(1-methylpyrazol-4-yl)-4-pyridyl] oxy}benzamide (154)

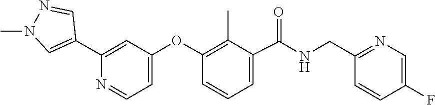 [SMILES: Cc1c(Oc2ccnc(-c3cnn(C)c3)c2)cccc1C(=O)NCc1ccc(F)cn1]

Compound 154 was synthesized from intermediate 119a (0.06 mmol) and 5-fluoro-2-pyridinemethanamine (0.10 mmol) as a white solid in 41% yield according to the general method C2. ¹H NMR (400 MHz, DMSO-d₆) δ (ppm): 8.99 (t, J=6.0 Hz, 1H), 8.52 (d, J=2.9 Hz, 1H), 8.36 (d, J=5.7 Hz, 1H), 8.24 (s, 1H), 7.96 (s, 1H), 7.73 (td, J=8.8 Hz, 3.0 Hz, 1H), 7.47 (dd, J=8.7 Hz, 4.5 Hz, 1H), 7.43-7.35 (m, 2H), 7.28-7.19 (m, 2H), 6.49 (dd, J=5.7 Hz, 2.4 Hz, 1H), 4.54 (d, J=6.0 Hz, 2H), 3.86 (s, 3H), 2.16 (s, 3H).

ESI-MS: 418.20 (M+H)⁺.

### N-[(6-dimethylphosphoryl-3-pyridyl)methyl]-2-methyl-3-{[2-(1-methylpyrazol-4-yl)-4-pyridyl]oxy}benzamide (155)

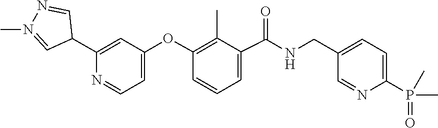

Compound 155 was synthesized from intermediate 119a (0.10 mmol) and (5-(aminomethyl)pyridin-2-yl)dimethylphosphine oxide (0.29 mmol) as a white solid in 78% yield according to the general method C2. ¹H NMR (400 MHz, DMSO-d₆) δ (ppm): 9.04 (t, J=5.9 Hz, 1H), 8.75 (s, 1H), 8.35 (d, J=5.7 Hz, 1H), 8.24 (s, 1H), 7.98-7.90 (m, 3H), 7.44-7.33 (m, 2H), 7.24-7.21 (m, 2H), 6.48 (dd, J=5.7 Hz, 2.4 Hz, 1H), 4.54 (d, J=5.9 Hz, 2H), 3.86 (s, 3H), 2.14 (s, 3H), 1.66 (s, 3H), 1.63 (s, 3H). ³¹P NMR (162 MHz, DMSO-d₆) δ (ppm): 33.89.

ESI-MS: 476.10 (M+H)⁺.

### N-[(3,5-difluorophenyl)methyl]-4-methyl-3-{[2-(1-methylpyrazol-4-yl)-4-pyridyl] oxy}benzamide (156)

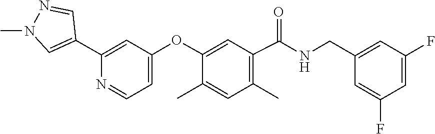 [SMILES: Cc1cc(C)c(C(=O)NCc2cc(F)cc(F)c2)cc1Oc1ccnc(-c2cnn(C)c2)c1]

Compound 156 was synthesized from intermediate 119b (0.10 mmol) and 3,5-difluorobenzylamine (0.15 mmol) as a white solid in 67% yield according to the general method C2. ¹H NMR (400 MHz, DMSO-d₆) δ (ppm): 9.10 (t, J=6.0 Hz, 1H), 8.36 (d, J=5.7 Hz, 1H), 8.25 (s, 1H), 7.97 (d, J=0.7 Hz, 1H), 7.79 (dd, J=7.9 Hz, 1.7 Hz, 1H), 7.64 (d, J=1.7 Hz, 1H), 7.51 (d, J=8.3 Hz, 1H), 7.29-7.20 (m, 1H), 7.13-7.05 (m, 1H), 7.04-6.98 (m, 2H), 6.55 (dd, J=5.7 Hz, 2.4 Hz, 1H), 4.46 (d, J=5.9 Hz, 2H), 3.86 (s, 3H), 2.20 (s, 3H).

ESI-MS: 435.00 (M+H)⁺.

### N-[(3,4-difluorophenyl)methyl]-4-methyl-3-{[2-(1-methylpyrazol-4-yl)-4-pyridyl] oxy}benzamide (157)

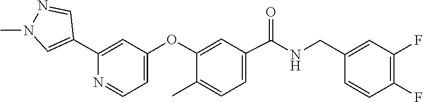 [SMILES: Cc1ccc(C(=O)NCc2ccc(F)c(F)c2)cc1Oc1ccnc(-c2cnn(C)c2)c1]

Compound 157 was synthesized from intermediate 119b (0.10 mmol) and 3,4-difluorobenzylamine (0.15 mmol) as a white solid in 45% yield according to the general method C2. ¹H NMR (400 MHz, DMSO-d₆) δ (ppm): 9.07 (t, J=5.9 Hz, 1H), 8.40-8.33 (m, 1H), 8.25 (s, 1H), 7.97 (d, J=0.7 Hz, 1H), 7.78 (dd, J=7.9 Hz, 1.7 Hz, 1H), 7.63 (d, J=1.7 Hz, 1H), 7.50 (d, J=8.4 Hz, 1H), 7.42-7.31 (m, 2H), 7.23 (d, J=2.1 Hz, 1H), 7.16-7.13 (m, 1H), 6.54 (dd, J=5.7 Hz, 2.4 Hz, 1H), 4.42 (d, J=5.9 Hz, 2H), 3.86 (s, 3H), 2.20 (s, 3H).

ESI-MS: 435.00 (M+H)⁺.

### N-[(4-chloro-3-fluoro-phenyl)methyl]-4-methyl-3-{[2-(1-methylpyrazol-4-yl)-4-pyridyl]oxy}benzamide (158)

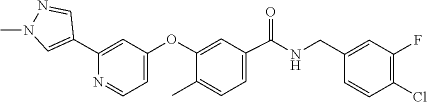 [SMILES: Cc1ccc(C(=O)NCc2ccc(Cl)c(F)c2)cc1Oc1ccnc(-c2cnn(C)c2)c1]

Compound 158 was synthesized from intermediate 119b (0.10 mmol) and 4-chloro-3-fluorobenzylamine (0.15 mmol) as a white solid in 57% yield according to the general method C2. ¹H NMR (400 MHz, DMSO-d₆) δ (ppm): 9.09 (t, J=6.0 Hz, 1H), 8.39-8.34 (m, 1H), 8.25 (s, 1H), 7.97 (d, J=0.7 Hz, 1H), 7.79 (dd, J=7.9 Hz, 1.7 Hz, 1H), 7.63 (d, J=1.7 Hz, 1H), 7.55-7.47 (m, 2H), 7.33 (dd, J=10.4 Hz, 1.9 Hz, 1H), 7.27-7.21 (m, 1H), 7.17 (dd, J=8.2 Hz, 1.3 Hz, 1H), 6.54 (dd, J=5.7 Hz, 2.4 Hz, 1H), 4.44 (d, J=5.9 Hz, 2H), 3.86 (s, 3H), 2.20 (s, 3H).

ESI-MS: 451.05 (M+H)⁺.

### N-[(4-chlorophenyl)methyl]-4-methyl-3-{[2-(1-methylpyrazol-4-yl)-4-pyridyl]oxy} benzamide (159)

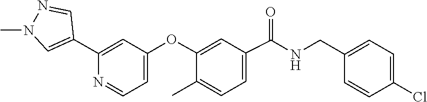 [SMILES: Cc1ccc(C(=O)NCc2ccc(Cl)cc2)cc1Oc1ccnc(-c2cnn(C)c2)c1]

Compound 159 was synthesized from intermediate 119b (0.10 mmol) and 4-chlorobenzylamine (0.15 mmol) as a white solid in 92% yield according to the general method C2. ¹H NMR (400 MHz, DMSO-d₆) δ (ppm): 9.07 (t, J=6.0 Hz, 1H), 8.38-8.34 (m, 1H), 8.25 (s, 1H), 7.97 (d, J=0.7 Hz, 1H), 7.78 (dd, J=7.9 Hz, 1.7 Hz, 1H), 7.63 (d, J=1.7 Hz, 1H), 7.50 (d, J=8.4 Hz, 1H), 7.40-7.29 (m, 4H), 7.23 (d, J=2.1 Hz, 1H), 6.54 (dd, J=5.7 Hz, 2.4 Hz, 1H), 4.43 (d, J=5.9 Hz, 2H), 3.86 (s, 3H), 2.20 (s, 3H).

ESI-MS: 433.00 (M+H)⁺.

### N-[(4-fluorophenyl)methyl]-4-methyl-3-{[2-(1-methylpyrazol-4-yl)-4-pyridyl]oxy} benzamide (352)

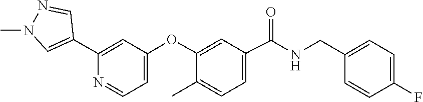 [SMILES: Cc1ccc(C(=O)NCc2ccc(F)cc2)cc1Oc1ccnc(-c2cnn(C)c2)c1]

Compound 352 was synthesized from intermediate 119b (0.08 mmol) and 4-fluorobenzylamine (0.12 mmol) as a white solid in 80% yield according to the general method C2. ¹H NMR (400 MHz, DMSO-d₆) δ (ppm): 9.05 (t, J=6.0 Hz, 1H), 8.36 (d, J=5.6 Hz, 1H), 8.25 (s, 1H), 7.97 (s, 1H), 7.78 (d, J=7.8 Hz, 1H), 7.63 (d, J=1.6 Hz, 1H), 7.50 (d, J=8.2 Hz, 1H), 7.36-7.31 (m, 2H), 7.23 (d, J=2.4 Hz, 1H), 7.16-7.10 (m, 2H), 6.54 (dd, J=5.7 Hz, 2.5 Hz, 1H), 4.42 (d, J=5.8 Hz, 2H), 3.86 (s, 3H), 2.20 (s, 3H).

ESI-MS: 417.10 (M+H)⁺.

### 4-fluoro-N-[(4-fluorophenyl)methyl]-3-{[2-(1-methylpyrazol-4-yl)-4-pyridyl]oxy} benzamide (353)

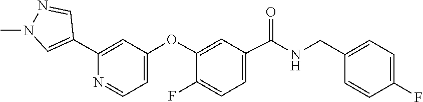 [SMILES: Cn1cc(-c2cc(Oc3cc(C(=O)NCc4ccc(F)cc4)ccc3F)ccn2)cn1]

Compound 353 was synthesized from intermediate 119i (0.08 mmol) and 4-fluorobenzylamine (0.12 mmol) as a white solid in 63% yield according to the general method C2. ¹H NMR (400 MHz, DMSO-d₆) δ (ppm): 9.14 (t, J=5.9 Hz, 1H), 8.40 (d, J=5.7 Hz, 1H), 8.28 (s, 1H), 7.99 (s, 1H), 7.94-7.87 (m, 2H), 7.61-7.56 (m, 1H), 7.37-7.30 (m, 3H), 7.19-7.10 (m, 2H), 6.71 (dd, J=5.7 Hz, 2.4 Hz, 1H), 4.44 (d, J=5.8 Hz, 2H), 3.86 (s, 3H).

ESI-MS: 421.05 (M+H)⁺.

### N-[(3,5-difluorophenyl)methyl]-2-methyl-5-{[2-(1-methylpyrazol-4-yl)-4-pyridyl] oxy}benzamide (160)

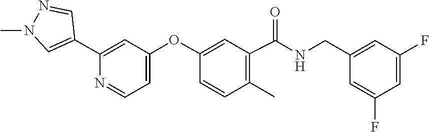 [SMILES: Cc1ccc(Oc2ccnc(-c3cnn(C)c3)c2)cc1C(=O)NCc1cc(F)cc(F)c1]

Compound 160 was synthesized from intermediate 119c (0.10 mmol) and 3,5-difluorobenzylamine (0.15 mmol) as a white solid in 90% yield according to the general method C2. ¹H NMR (400 MHz, DMSO-d₆) δ (ppm): 8.92 (t, J=6.0 Hz, 1H), 8.40-8.35 (m, 1H), 8.25 (s, 1H), 7.96 (d, J=0.7 Hz, 1H), 7.36 (d, J=8.4 Hz, 1H), 7.27-7.23 (m, 2H), 7.19 (dd, J=8.2 Hz, 2.6 Hz, 1H), 7.14-7.07 (m, 1H), 7.07-7.02 (m, 2H), 6.66 (dd, J=5.7 Hz, 2.4 Hz, 1H), 4.44 (d, J=6.0 Hz, 2H), 3.86 (s, 3H), 2.36 (s, 3H).

ESI-MS: 435.00 (M+H)⁺.

### N-[(3,4-difluorophenyl)methyl]-2-methyl-5-{[2-(1-methylpyrazol-4-yl)-4-pyridyl] oxy}benzamide (161)

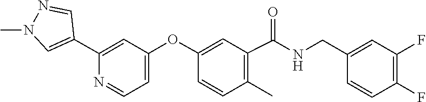 [SMILES: Cc1ccc(Oc2ccnc(-c3cnn(C)c3)c2)cc1C(=O)NCc1ccc(F)c(F)c1]

Compound 161 was synthesized from intermediate 119c (0.10 mmol) and 3,4-difluorobenzylamine (0.15 mmol) as a white solid in 41% yield according to the general method C2. ¹H NMR (400 MHz, DMSO-d₆) δ (ppm): 8.90 (t, J=6.0 Hz, 1H), 8.39-8.35 (m, 1H), 8.25 (s, 1H), 7.96 (d, J=0.7 Hz, 1H), 7.44-7.34 (m, 3H), 7.25 (d, J=2.1 Hz, 1H), 7.22 (d, J=2.6 Hz, 1H), 7.20-7.15 (m, 2H), 6.65 (dd, J=5.7 Hz, 2.4 Hz, 1H), 4.41 (d, J=5.9 Hz, 2H), 3.86 (s, 3H), 2.35 (s, 3H).

ESI-MS: 435.05 (M+H)⁺.

### N-[(4-chloro-3-fluoro-phenyl)methyl]-2-methyl-5-{[2-(1-methylpyrazol-4-yl)-4-pyridyl]oxy}benzamide (162)

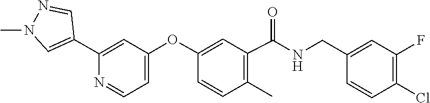 [SMILES: Cc1ccc(Oc2ccnc(-c3cnn(C)c3)c2)cc1C(=O)NCc1ccc(Cl)c(F)c1]

Compound 162 was synthesized from intermediate 119c (0.10 mmol) and 4-chloro-3-fluorobenzylamine (0.15 mmol) as a white solid in 60% yield according to the general method C2. ¹H NMR (400 MHz, DMSO-d₆) δ (ppm): 8.92 (t, J=6.0 Hz, 1H), 8.40-8.35 (m, 1H), 8.25 (s, 1H), 7.96 (d, J=0.7 Hz, 1H), 7.54 (t, J=8.0 Hz, 1H), 7.37-7.33 (m, 2H), 7.28-7.15 (m, 4H), 6.65 (dd, J=5.7 Hz, 2.4 Hz, 1H), 4.43 (d, J=6.0 Hz, 2H), 3.86 (s, 3H), 2.35 (s, 3H).

ESI-MS: 451.05 (M+H)⁺.

### N-[(4-chlorophenyl)methyl]-2-methyl-5-{[2-(1-methylpyrazol-4-yl)-4-pyridyl]oxy} benzamide (163)

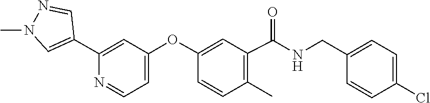 [SMILES: Cc1ccc(Oc2ccnc(-c3cnn(C)c3)c2)cc1C(=O)NCc1ccc(Cl)cc1]

Compound 163 was synthesized from intermediate 119c (0.10 mmol) and 4-chlorobenzylamine (0.15 mmol) as a white solid in 79% yield according to the general method C2. ¹H NMR (400 MHz, DMSO-d₆) δ (ppm): 8.98 (t, J=6.0 Hz, 1H), 8.48-8.45 (m, 1H), 8.34 (s, 1H), 8.05 (d, J=0.7 Hz, 1H), 7.51-7.41 (m, 5H), 7.34 (d, J=2.1 Hz, 1H), 7.30-7.23 (m, 2H), 6.74 (dd, J=5.7 Hz, 2.4 Hz, 1H), 4.50 (d, J=6.0 Hz, 2H), 3.95 (s, 3H), 2.44 (s, 3H).

ESI-MS: 433.00 (M+H)⁺.

### N-[(2-methoxyphenyl)methyl]-2-methyl-5-{[2-(1-methylpyrazol-4-yl)-4-pyridyl] oxy}benzamide (351)

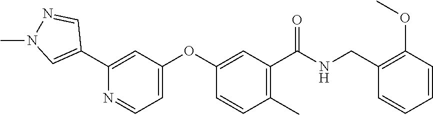 [SMILES: COc1ccccc1CNC(=O)c1cc(Oc2ccnc(-c3cnn(C)c3)c2)ccc1C]

Compound 351 was synthesized from intermediate 119c (0.07 mmol) and 2-methoxybenzylamine (0.10 mmol) as a white solid in 71% yield according to the general method C2. ¹H NMR (400 MHz, DMSO-d₆) δ (ppm): 8.70 (t, J=5.8 Hz, 1H), 8.37 (d, J=5.7 Hz, 1H), 8.26 (s, 1H), 7.97 (s, 1H), 7.35 (d, J=8.2 Hz, 1H), 7.28-7.21 (m, 3H), 7.20-7.16 (m, 2H), 6.98 (d, J=8.2 Hz, 1H), 6.90 (t, J=7.4 Hz, 1H), 6.66 (dd, J=5.7 Hz, 2.4 Hz, 1H), 4.39 (d, J=5.9 Hz, 2H), 3.86 (s, 3H), 3.79 (s, 3H), 2.36 (s, 3H).

ESI-MS: 429.10 (M+H)⁺.

### 2-methyl-5-{[2-(1-methylpyrazol-4-yl)-4-pyridyl]oxy}-N-{[3-(trifluoromethyl) phenyl]methyl}benzamide (354)

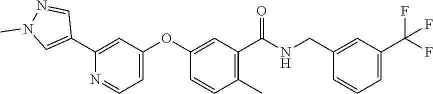 [SMILES: Cc1ccc(Oc2ccnc(-c3cnn(C)c3)c2)cc1C(=O)NCc1cccc(C(F)(F)F)c1]

Compound 354 was synthesized from intermediate 119c (0.07 mmol) and 3-trifluoromethylbenzylamine (0.10 mmol) as a white solid in 73% yield according to the general method C2. ¹H NMR (400 MHz, DMSO-d₆) δ (ppm): 8.97 (t, J=6.0 Hz, 1H), 8.37 (d, J=5.7 Hz, 1H), 8.25 (s, 1H), 7.96 (s, 1H), 7.72-7.53 (m, 4H), 7.36 (d, J=7.8 Hz, 1H), 7.26 (d, J=2.3 Hz, 1H), 7.21-7.16 (m, 2H), 6.66 (dd, J=5.7 Hz, 2.4 Hz, 1H), 4.52 (d, J=6.0 Hz, 2H), 3.86 (s, 3H), 2.35 (s, 3H).

ESI-MS: 467.10 (M+H)⁺.

### N-[(3,4-difluorophenyl)methyl]-2-methoxy-5-{[2-(1-methylpyrazol-4-yl)-4-pyridyl] oxy}benzamide (359)

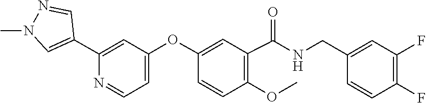 [SMILES: COc1ccc(Oc2ccnc(-c3cnn(C)c3)c2)cc1C(=O)NCc1ccc(F)c(F)c1]

Compound 359 was synthesized from intermediate 119j (0.08 mmol) and 3,4-difluorobenzylamine (0.12 mmol) as a white solid in 72% yield according to the general method C2. ¹H NMR (400 MHz, DMSO-d₆) δ (ppm): 8.87 (t, J=6.1 Hz, 1H), 8.36 (d, J=5.7 Hz, 1H), 8.24 (s, 1H), 7.95 (d, J=0.7 Hz, 1H), 7.49 (d, J=3.1 Hz, 1H), 7.43-7.32 (m, 3H), 7.28-7.15 (m, 3H), 6.61 (dd, J=5.7 Hz, 2.4 Hz, 1H), 4.47 (d, J=6.1 Hz, 2H), 3.94 (s, 3H), 3.86 (s, 3H).

ESI-MS: 451.10 (M+H)⁺.

### N-[(3,5-difluorophenyl)methyl]-3-methyl-5-{[2-(1-methylpyrazol-4-yl)-4-pyridyl] oxy}benzamide (164)

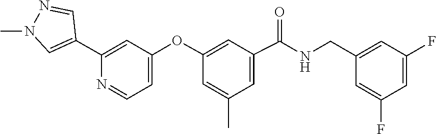 [SMILES: Cc1cc(Oc2ccnc(-c3cnn(C)c3)c2)cc(C(=O)NCc2cc(F)cc(F)c2)c1]

Compound 164 was synthesized from intermediate 119d (0.10 mmol) and 3,5-difluorobenzylamine (0.15 mmol) as a white solid in 43% yield according to the general method C2. ¹H NMR (400 MHz, DMSO-d₆) δ (ppm): 9.12 (t, J=5.9 Hz, 1H), 8.39 (d, J=5.7 Hz, 1H), 8.27 (s, 1H), 7.97 (d, J=0.5 Hz, 1H), 7.66 (s, 1H), 7.49 (s, 1H), 7.28 (d, J=2.2 Hz, 1H), 7.22 (s, 1H), 7.13-7.07 (m, 1H), 7.04-6.99 (m, 2H), 6.68 (dd, J=5.7 Hz, 2.4 Hz, 1H), 4.47 (d, J=5.9 Hz, 2H), 3.86 (s, 3H), 2.40 (s, 3H).

ESI-MS: 435.00 (M+H)⁺.

### N-[(3,4-difluorophenyl)methyl]-3-methyl-5-{[2-(1-methylpyrazol-4-yl)-4-pyridyl] oxy}benzamide (165)

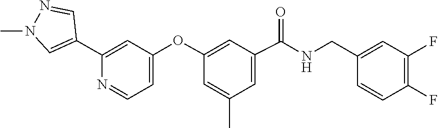 [SMILES: Cc1cc(Oc2ccnc(-c3cnn(C)c3)c2)cc(C(=O)NCc2ccc(F)c(F)c2)c1]

Compound 165 was synthesized from intermediate 119d (0.10 mmol) and 3,4-difluorobenzylamine (0.15 mmol) as a white solid in 29% yield according to the general method C2. ¹H NMR (400 MHz, DMSO-d₆) δ (ppm): 9.09 (t, J=5.9 Hz, 1H), 8.39 (d, J=5.7 Hz, 1H), 8.26 (s, 1H), 7.97 (d, J=0.7 Hz, 1H), 7.66-7.64 (m, 1H), 7.48-7.47 (m, 1H), 7.42-7.32 (m, 2H), 7.28 (d, J=2.1 Hz, 1H), 7.22-7.21 (m, 1H), 7.19-7.11 (m, 1H), 6.67 (dd, J=5.7 Hz, 2.4 Hz, 1H), 4.43 (d, J=5.9 Hz, 2H), 3.86 (s, 3H), 2.39 (s, 3H).

ESI-MS: 435.00 (M+H)⁺.

### N-[(4-chloro-3-fluoro-phenyl)methyl]-3-methyl-5-{[2-(1-methylpyrazol-4-yl)-4-pyridyl]oxy}benzamide (166)

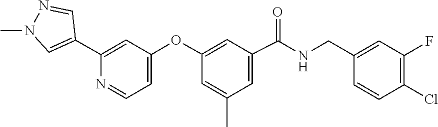 [SMILES: Cc1cc(Oc2ccnc(-c3cnn(C)c3)c2)cc(C(=O)NCc2ccc(Cl)c(F)c2)c1]

Compound 166 was synthesized from intermediate 119d (0.10 mmol) and 4-chloro-3-fluorobenzylamine (0.15 mmol) as a white solid in 46% yield according to the general method C2. ¹H NMR (400 MHz, DMSO-d₆) δ (ppm): 9.11 (t, J=5.9 Hz, 1H), 8.41-8.36 (m, 1H), 8.26 (s, 1H), 7.97 (d, J=0.7 Hz, 1H), 7.66-7.65 (m, 1H), 7.53 (t, J=8.0 Hz, 1H), 7.48-7.47 (m, 1H), 7.33 (dd, J=10.4 Hz, 1.9 Hz, 1H), 7.28 (d, J=2.1 Hz, 1H), 7.22-7.21 (m, 1H), 7.18 (dd, J=8.3 Hz, 1.3 Hz, 1H), 6.67 (dd, J=5.7 Hz, 2.4 Hz, 1H), 4.45 (d, J=5.9, 2H), 3.86 (s, 3H), 2.39 (s, 3H).

ESI-MS: 451.05 (M+H)⁺.

### N-[(4-chlorophenyl)methyl]-3-methyl-5-{[2-(1-methylpyrazol-4-yl)-4-pyridyl]oxy} benzamide (167)

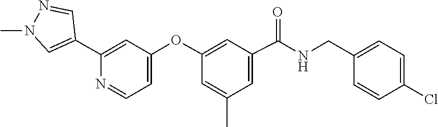 [SMILES: Cc1cc(Oc2ccnc(-c3cnn(C)c3)c2)cc(C(=O)NCc2ccc(Cl)cc2)c1]

Compound 167 was synthesized from intermediate 119d (0.10 mmol) and 4-chlorobenzylamine (0.15 mmol) as a white solid in 53% yield according to the general method C2. ¹H NMR (400 MHz, DMSO-d₆) δ (ppm): 9.09 (t, J=5.9 Hz, 1H), 8.41-8.37 (m, 1H), 8.26 (s, 1H), 7.97 (d, J=0.7 Hz, 1H), 7.66-7.65 (m, 1H), 7.49-7.45 (m, 1H), 7.40-7.30 (m, 4H), 7.28 (d, J=2.0 Hz, 1H), 7.22-7.21 (m, 1H), 6.66 (dd, J=5.7 Hz, 2.4 Hz, 1H), 4.43 (d, J=5.9 Hz, 2H), 3.86 (s, 3H), 2.39 (s, 3H).

ESI-MS: 433.00 (M+H)⁺.

### N-[(3-fluorophenyl)methyl]-2-methyl-3-({2-[(1-methylpyrazol-3-yl)amino]-4-pyridyl}oxy)benzamide (168)

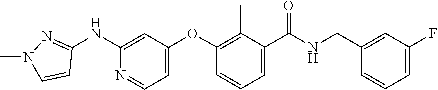 [SMILES: Cc1c(Oc2ccnc(Nc3ccn(C)n3)c2)cccc1C(=O)NCc1cccc(F)c1]

Compound 168 was synthesized from intermediate 119e (0.09 mmol) and 3-fluorobenzylamine (0.14 mmol) as a white solid in 53% yield according to the general method C2. ¹H NMR (400 MHz, DMSO-d₆) δ (ppm): 9.17 (s, 1H), 8.94 (t, J=6.0 Hz, 1H), 7.98 (d, J=5.7 Hz, 1H), 7.46 (d, J=2.2 Hz, 1H), 7.42-7.31 (m, 3H), 7.22-7.13 (m, 3H), 7.08 (td, J=8.3 Hz, 2.1 Hz, 1H), 6.88 (d, J=2.1 Hz, 1H), 6.18 (dd, J=5.8 Hz, 2.3 Hz, 1H), 6.15 (d, J=2.2 Hz, 1H), 4.47 (d, J=6.0 Hz, 2H), 3.67 (s, 3H), 2.14 (s, 3H).

ESI-MS: 432.15 (M+H)⁺.

### N-[(4-fluorophenyl)methyl]-2-methyl-3-({2-[(1-methylpyrazol-3-yl)amino]-4-pyridyl}oxy)benzamide (169)

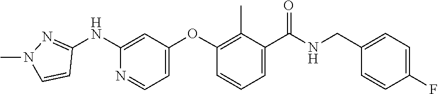 [SMILES: Cc1c(Oc2ccnc(Nc3ccn(C)n3)c2)cccc1C(=O)NCc1ccc(F)cc1]

Compound 169 was synthesized from intermediate 119e (0.09 mmol) and 4-fluorobenzylamine (0.14 mmol) as a white solid in 50% yield according to the general method C2. ¹H NMR (400 MHz, DMSO-d₆) δ (ppm): 9.17 (s, 1H), 8.91 (t, J=6.0 Hz, 1H), 7.98 (d, J=5.8 Hz, 1H), 7.46 (d, J=2.2 Hz, 1H), 7.42-7.27 (m, 4H), 7.21-7.13 (m, 3H), 6.88 (d, J=2.1 Hz, 1H), 6.17 (dd, J=5.8 Hz, 2.3 Hz, 1H), 6.15 (d, J=2.2 Hz, 1H), 4.43 (d, J=6.0 Hz, 2H), 3.67 (s, 3H), 2.13 (s, 3H).

ESI-MS: 432.15 (M+H)⁺.

### N-[(3-chlorophenyl)methyl]-2-methyl-3-({2-[(1-methylpyrazol-3-yl)amino]-4-pyridyl}oxy)benzamide (170)

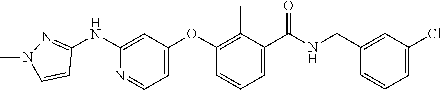 [SMILES: Cc1c(Oc2ccnc(Nc3ccn(C)n3)c2)cccc1C(=O)NCc1cccc(Cl)c1]

Compound 170 was synthesized from intermediate 119e (0.09 mmol) and 3-chlorobenzylamine (0.14 mmol) as a white solid in 63% yield according to the general method C2. ¹H NMR (400 MHz, DMSO-d₆) δ (ppm): 9.17 (s, 1H), 8.95 (t, J=6.1 Hz, 1H), 7.98 (d, J=5.8 Hz, 1H), 7.46 (d, J=2.2 Hz, 1H), 7.43-7.28 (m, 6H), 7.18 (dd, J=7.8 Hz, 1.2 Hz, 1H), 6.88 (d, J=2.1 Hz, 1H), 6.18 (dd, J=5.8 Hz, 2.3 Hz, 1H), 6.15 (d, J=2.2 Hz, 1H), 4.46 (d, J=6.0 Hz, 2H), 3.67 (s, 3H), 2.14 (s, 3H).

### N-[(4-chlorophenyl)methyl]-2-methyl-3-({2-[(1-methylpyrazol-3-yl)amino]-4-pyridyl}oxy)benzamide (171)

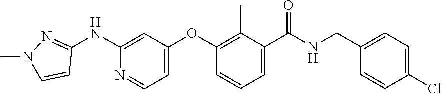 [SMILES: Cc1c(Oc2ccnc(Nc3ccn(C)n3)c2)cccc1C(=O)NCc1ccc(Cl)cc1]

Compound 171 was synthesized from intermediate 119e (0.09 mmol) and 4-chlorobenzylamine (0.14 mmol) as a white solid in 49% yield according to the general method C2. ¹H NMR (400 MHz, DMSO-d₆) δ (ppm): 9.17 (s, 1H), 8.93 (t, J=6.0 Hz, 1H), 7.98 (d, J=5.8 Hz, 1H), 7.46 (d, J=2.2 Hz, 1H), 7.43-7.28 (m, 6H), 7.18 (dd, J=7.8 Hz, 1.3 Hz, 1H), 6.88 (d, J=2.1 Hz, 1H), 6.17 (dd, J=5.8 Hz, 2.3 Hz, 1H), 6.15 (d, J=2.2 Hz, 1H), 4.43 (d, J=6.0 Hz, 2H), 3.67 (s, 3H), 2.13 (s, 3H).

ESI-MS: 448.15 (M+H)⁺.

### 2-methyl-3-({2-[(1-methylpyrazol-3-yl)amino]-4-pyridyl}oxy)-N-{[6-(trifluoro methyl)-3-pyridyl]methyl}benzamide (172)

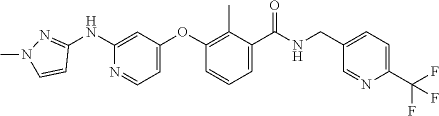 [SMILES: Cc1c(Oc2ccnc(Nc3ccn(C)n3)c2)cccc1C(=O)NCc1ccc(C(F)(F)F)nc1]

Compound 172 was synthesized from intermediate 119e (0.09 mmol) and [6-(trifluoromethyl)-3-pyridyl]methanamine (0.14 mmol) as a white solid in 57% yield according to the general method C2. ¹H NMR (400 MHz, DMSO-d₆) δ (ppm): 9.17 (s, 1H), 9.05 (t, J=5.9 Hz, 1H), 8.76-8.75 (m, 1H), 8.04 (d, J=8.1 Hz, 1H), 7.98 (d, J=5.8 Hz, 1H), 7.91 (d, J=8.0 Hz, 1H), 7.46 (d, J=2.2 Hz, 1H), 7.40-7.33 (m, 2H), 7.19 (dd, J=7.0 Hz, 2.4 Hz, 1H), 6.88 (d, J=2.1 Hz, 1H), 6.18 (dd, J=5.8 Hz, 2.3 Hz, 1H), 6.15 (d, J=2.2 Hz, 1H), 4.58 (d, J=5.9 Hz, 2H), 3.67 (s, 3H), 2.14 (s, 3H).

ESI-MS: 483.15 (M+H)⁺.

### N-(imidazo[1,2-a]pyridin-6-ylmethyl)-2-methyl-3-({2-[(1-methylpyrazol-3-yl)amino]-4-pyridyl}oxy)benzamide (173)

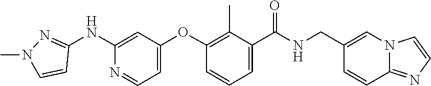 [SMILES: Cc1c(Oc2ccnc(Nc3ccn(C)n3)c2)cccc1C(=O)NCc1ccc2nccn2c1]

Compound 173 was synthesized from intermediate 119e (0.09 mmol) and imidazo[1,2-a]pyridin-6-ylmethanamine (0.14 mmol) as a white solid in 50% yield according to the general method C2. ¹H NMR (400 MHz, DMSO-d₆) δ (ppm): 9.17 (s, 1H), 8.93 (t, J=5.9 Hz, 1H), 8.50-8.49 (m, 1H), 7.99-7.95 (m, 2H), 7.57-7.54 (m, 2H), 7.46 (d, J=2.2 Hz, 1H), 7.38-7.31 (m, 2H), 7.25 (dd, J=9.3 Hz, 1.7 Hz, 1H), 7.18 (dd, J=7.4 Hz, 1.8 Hz, 1H), 6.88 (d, J=2.1 Hz, 1H), 6.17 (dd, J=5.8 Hz, 2.3 Hz, 1H), 6.15 (d, J=2.2 Hz, 1H), 4.44 (d, J=5.8 Hz, 2H), 3.67 (s, 3H), 2.13 (s, 3H).

ESI-MS: 454.15 (M+H)⁺.

### N-(imidazo[1,2-a]pyridin-7-ylmethyl)-2-methyl-3-({2-[(1-methylpyrazol-3-yl)amino]-4-pyridyl}oxy)benzamide (174)

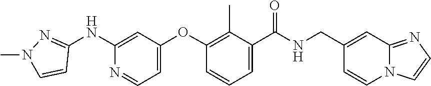 [SMILES: Cc1c(Oc2ccnc(Nc3ccn(C)n3)c2)cccc1C(=O)NCc1ccn2ccnc2c1]

Compound 174 was synthesized from intermediate 119e (0.09 mmol) and imidazo[1,2-a]pyridin-7-ylmethanamine (0.14 mmol) as a white solid in 41% yield according to the general method C2. ¹H NMR (400 MHz, DMSO-d₆) δ (ppm): 9.17 (s, 1H), 8.98 (t, J=6.0 Hz, 1H), 8.51 (dd, J=7.0 Hz, 0.7 Hz, 1H), 7.98 (d, J=5.8 Hz, 1H), 7.90 (s, 1H), 7.53 (d, J=1.1 Hz, 1H), 7.46 (d, J=2.2 Hz, 1H), 7.44 (s, 1H), 7.40-7.33 (m, 2H), 7.19 (dd, J=7.4 Hz, 1.8 Hz, 1H), 6.90-6.87 (m, 2H), 6.18 (dd, J=5.8 Hz, 2.3 Hz, 1H), 6.15 (d, J=2.2 Hz, 1H), 4.49 (d, J=5.9 Hz, 2H), 3.67 (s, 3H), 2.16 (s, 3H).

ESI-MS: 454.15 (M+H)⁺.

### N-[(5-fluoro-3-pyridyl)methyl]-2-methyl-3-({2-[(1-methylpyrazol-3-yl)amino]-4-pyridyl}oxy)benzamide (175)

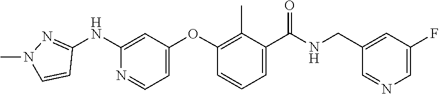 [SMILES: Cc1c(Oc2ccnc(Nc3ccn(C)n3)c2)cccc1C(=O)NCc1cncc(F)c1]

Compound 175 was synthesized from intermediate 119e (0.06 mmol) and 5-fluoro-3-pyridinemethanamine (0.09 mmol) as a white solid in 37% yield according to the general method C2. ¹H NMR (400 MHz, DMSO-d₆) δ (ppm): 9.17 (s, 1H), 9.00 (t, J=5.9 Hz, 1H), 8.49 (d, J=2.8 Hz, 1H), 8.47-8.46 (m, 1H), 7.98 (d, J=5.8 Hz, 1H), 7.69-7.65 (m, 1H), 7.46 (d, J=2.2 Hz, 1H), 7.39-7.32 (m, 2H), 7.19 (dd, J=7.2 Hz, 2.0 Hz, 1H), 6.88 (d, J=2.1 Hz, 1H), 6.18 (dd, J=5.8 Hz, 2.3 Hz, 1H), 6.15 (d, J=2.2 Hz, 1H), 4.52 (d, J=5.9 Hz, 2H), 3.67 (s, 3H), 2.13 (s, 3H).

ESI-MS: 433.20 (M+H)⁺.

### N-[(3,4-difluorophenyl)methyl]-2-fluoro-5-({2-[(1-methylpyrazol-3-yl)amino]-4-pyridyl}oxy)benzamide (355)

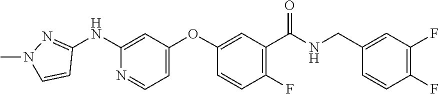 [SMILES: Cn1ccc(Nc2cc(Oc3ccc(F)c(C(=O)NCc4ccc(F)c(F)c4)c3)ccn2)n1]

Compound 355 was synthesized from intermediate 119k (0.08 mmol) and 3,4-difluorobenzylamine (0.11 mmol) as a white solid in 30% yield according to the general method C2. ¹H NMR (400 MHz, DMSO-d₆) δ (ppm): 9.21 (s, 1H), 8.98 (t, J=5.0 Hz, 1H), 8.01 (d, J=5.7 Hz, 1H), 7.49-7.33 (m, 6H), 7.22-7.14 (m, 1H), 6.93 (d, J=2.1 Hz, 1H), 6.30 (dd, J=5.7 Hz, 2.3 Hz, 1H), 6.16 (d, J=2.2 Hz, 1H), 4.44 (d, J=6.0 Hz, 2H), 3.67 (s, 3H).

ESI-MS: 454.00 (M+H)⁺.

### N-[(5-fluoro-6-methoxy-3-pyridyl)methyl]-2-methyl-3-({2-[(1-methylpyrazol-4-yl)amino]-4-pyridyl}oxy)benzamide (176)

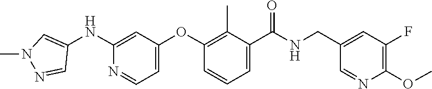

Compound 176 was synthesized from intermediate 119e (0.06 mmol) and (5-fluoro-6-methoxy-3-pyridyl) methanamine (0.09 mmol) as a white solid in 59% yield according to the general method C2. ¹H NMR (400 MHz, DMSO-d₆) δ (ppm): 9.17 (s, 1H), 8.90 (t, J=5.9 Hz, 1H), 7.98-7.96 (m, 2H), 7.64 (dd, J=11.4 Hz, 1.9 Hz, 1H), 7.46 (d, J=2.2 Hz, 1H), 7.37-7.28 (m, 2H), 7.17 (dd, J=7.8 Hz, 1.2 Hz, 1H), 6.87 (d, J=2.1 Hz, 1H), 6.17 (dd, J=5.8 Hz, 2.3 Hz, 1H), 6.14 (d, J=2.2 Hz, 1H), 4.41 (d, J=5.9 Hz, 2H), 3.93 (s, 3H), 3.67 (s, 3H), 2.12 (s, 3H).

ESI-MS: 463.25 (M+H)⁺.

### N-[(5-fluoro-2-methoxy-3-pyridyl)methyl]-2-methyl-3-({2-[(1-methylpyrazol-4-yl)amino]-4-pyridyl}oxy)benzamide (177)

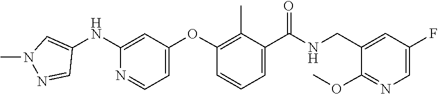 [SMILES: COc1ncc(F)cc1CNC(=O)c1cccc(Oc2ccnc(Nc3cnn(C)c3)c2)c1C]

Compound 177 was synthesized from intermediate 119e (0.06 mmol) and 5-fluoro-3-pyridinemethanamine (0.09 mmol) as a white solid in 48% yield according to the general method C2. ¹H NMR (400 MHz, DMSO-d₆) δ (ppm): 9.17 (s, 1H), 8.84 (t, J=5.8 Hz, 1H), 8.07 (d, J=3.0 Hz, 1H), 7.98 (d, J=5.8 Hz, 1H), 7.55 (dd, J=8.6 Hz, 3.0 Hz, 1H), 7.46 (d, J=2.2 Hz, 1H), 7.39-7.34 (m, 2H), 7.21-7.17 (m, 1H), 6.88 (d, J=2.1 Hz, 1H), 6.18 (dd, J=5.8 Hz, 2.3 Hz, 1H), 6.15 (d, J=2.2 Hz, 1H), 4.38 (d, J=5.7 Hz, 2H), 3.91 (s, 3H), 3.67 (s, 3H), 2.14 (s, 3H).

ESI-MS: 463.20 (M+H)⁺.

### N-(imidazo[1,2-a]pyridin-6-ylmethyl)-2-methyl-3-({2-[(1-methylpyrazol-4-yl)amino]-4-pyridyl}oxy)benzamide (178)

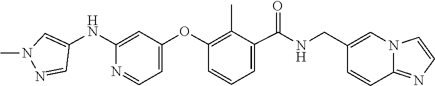 [SMILES: Cc1c(Oc2ccnc(Nc3cnn(C)c3)c2)cccc1C(=O)NCc1ccc2nccn2c1]

Compound 178 was synthesized from intermediate 119f (0.09 mmol) and imidazo[1,2-a]pyridin-6-ylmethanamine (0.14 mmol) as a white solid in 53% yield according to the general method C2. ¹H NMR (400 MHz, DMSO-d₆) δ (ppm): 8.95 (t, J=5.9 Hz, 1H), 8.74 (s, 1H), 8.50 (s, 1H), 7.99-7.96 (m, 2H), 7.86 (s, 1H), 7.57-7.55 (m, 2H), 7.38-7.31 (m, 3H), 7.24 (dd, J=9.3 Hz, 1.6 Hz, 1H), 7.19 (dd, J=7.4 Hz, 1.8 Hz, 1H), 6.23 (dd, J=5.8 Hz, 2.2 Hz, 1H), 5.93 (d, J=2.1 Hz, 1H), 4.44 (d, J=5.9 Hz, 2H), 3.76 (s, 3H), 2.12 (s, 3H).

ESI-MS: 454.15 (M+H)⁺.

### N-(imidazo[1,2-a]pyridin-7-ylmethyl)-2-methyl-3-({2-[(1-methylpyrazol-4-yl)amino]-4-pyridyl}oxy)benzamide (179)

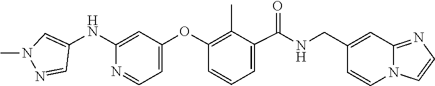 [SMILES: Cc1c(Oc2ccnc(Nc3cnn(C)c3)c2)cccc1C(=O)NCc1ccn2ccnc2c1]

Compound 179 was synthesized from intermediate 119f (0.09 mmol) and imidazo[1,2-a]pyridin-7-ylmethanamine (0.14 mmol) as a white solid in 46% yield according to the general method C2. ¹H NMR (400 MHz, DMSO-d₆) δ (ppm): 9.00 (t, J=6.0 Hz, 1H), 8.75 (s, 1H), 8.51 (d, J=7.0 Hz, 1H), 7.99 (d, J=5.8 Hz, 1H), 7.90 (s, 1H), 7.86 (s, 1H), 7.53 (d, J=1.1 Hz, 1H), 7.44 (s, 1H), 7.40-7.33 (m, 2H), 7.31 (s, 1H), 7.21 (dd, J=7.3 Hz, 2.0 Hz, 1H), 6.88 (dd, J=7.0 Hz, 1.6 Hz, 1H), 6.24 (dd, J=5.8 Hz, 2.2 Hz, 1H), 5.94 (d, J=2.2 Hz, 1H), 4.49 (d, J=5.9 Hz, 2H), 3.76 (s, 3H), 2.15 (s, 3H).

ESI-MS: 454.15 (M+H)⁺.

### 2-methyl-3-({2-[(1-methylpyrazol-4-yl)amino]-4-pyridyl}oxy)-N-{[6-(trifluoro methyl)-3-pyridyl]methyl} benzamide (180)

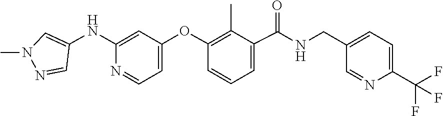 [SMILES: Cc1c(Oc2ccnc(Nc3cnn(C)c3)c2)cccc1C(=O)NCc1ccc(C(F)(F)F)nc1]

Compound 180 was synthesized from intermediate 119f (0.09 mmol) and [6-(trifluoromethyl)-3-pyridyl]methanamine (0.14 mmol) as a white solid in 58% yield according to the general method C2. ¹H NMR (400 MHz, DMSO-d₆) δ (ppm): 9.07 (t, J=5.9 Hz, 1H), 8.76-8.74 (m, 2H), 8.04 (dd, J=8.0 Hz, 1.5 Hz, 1H), 7.99 (d, J=5.8 Hz, 1H), 7.92 (d, J=8.1 Hz, 1H), 7.86 (s, 1H), 7.39-7.34 (m, 2H), 7.31 (s, 1H), 7.21 (dd, J=6.7 Hz, 2.6 Hz, 1H), 6.24 (dd, J=5.8 Hz, 2.2 Hz, 1H), 5.93 (d, J=2.2 Hz, 1H), 4.58 (d, J=5.9 Hz, 2H), 3.76 (s, 3H), 2.12 (s, 3H).

ESI-MS: 483.15 (M+H)⁺.

### N-[(5-fluoro-3-pyridyl)methyl]-2-methyl-3-({2-[(1-methylpyrazol-4-yl)amino]-4-pyridyl}oxy)benzamide (181)

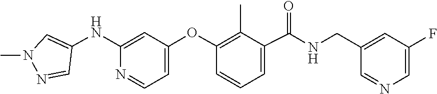 [SMILES: Cc1c(Oc2ccnc(Nc3cnn(C)c3)c2)cccc1C(=O)NCc1cncc(F)c1]

Compound 181 was synthesized from intermediate 119f (0.09 mmol) and 5-fluoro-3-pyridinemethanamine (0.14 mmol) as a white solid in 73% yield according to the general method C2. ¹H NMR (400 MHz, DMSO-d₆) δ (ppm): 9.00 (t, J=5.9 Hz, 1H), 8.73 (s, 1H), 8.49 (d, J=2.8 Hz, 1H), 8.47-8.46 (m, 1H), 7.99 (d, J=5.8 Hz, 1H), 7.86 (s, 1H), 7.69-7.65 (m, 1H), 7.39-7.33 (m, 2H), 7.31 (d, J=0.6 Hz, 1H), 7.20 (dd, J=7.2 Hz, 2.1 Hz, 1H), 6.23 (dd, J=5.8 Hz, 2.2 Hz, 1H), 5.94 (d, J=2.2 Hz, 1H), 4.52 (d, J=5.9 Hz, 2H), 3.77 (s, 3H), 2.12 (s, 3H).

ESI-MS: 433.20 (M+H)⁺.

### N-[(5-fluoro-6-methoxy-3-pyridyl)methyl]-2-methyl-3-({2-[(1-methylpyrazol-4-yl)amino]-4-pyridyl}oxy)benzamide (182)

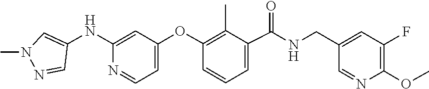

Compound 182 was synthesized from intermediate 119f (0.09 mmol) and (5-fluoro-6-methoxy-3-pyridyl) methanamine (0.14 mmol) as a white solid in 86% yield according to the general method C2. ¹H NMR (400 MHz, DMSO-d₆) δ (ppm): 8.90 (t, J=5.8 Hz, 1H), 8.72 (s, 1H), 7.99-7.97 (m, 2H), 7.86 (s, 1H), 7.64 (dd, J=11.4 Hz, 1.9 Hz, 1H), 7.37-7.29 (m, 3H), 7.19 (dd, J=7.8 Hz, 1.3 Hz, 1H), 6.23 (dd, J=5.8 Hz, 2.2 Hz, 1H), 5.94 (d, J=2.1 Hz, 1H), 4.41 (d, J=5.9 Hz, 2H), 3.93 (s, 3H), 3.77 (s, 3H), 2.11 (s, 3H).

ESI-MS: 463.20 (M+H)⁺.

### N-[(5-fluoro-2-methoxy-3-pyridyl)methyl]-2-methyl-3-({2-[(1-methylpyrazol-4-yl)amino]-4-pyridyl}oxy)benzamide (183)

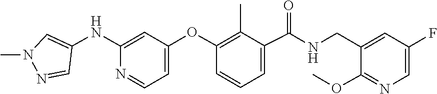 [SMILES: COc1ncc(F)cc1CNC(=O)c1cccc(Oc2ccnc(Nc3cnn(C)c3)c2)c1C]

Compound 183 was synthesized from intermediate 119f (0.09 mmol) and 5-fluoro-2-methoxy-3-pyridinemethanamine (0.14 mmol) as a white solid in 70% yield according to the general method C2. ¹H NMR (400 MHz, DMSO-d₆) δ (ppm): 8.84 (t, J=5.8 Hz, 1H), 8.73 (s, 1H), 8.07 (d, J=3.0 Hz, 1H), 7.99 (d, J=5.8 Hz, 1H), 7.86 (s, 1H), 7.55 (dd, J=8.6 Hz, 3.0 Hz, 1H), 7.39-7.34 (m, 2H), 7.31 (d, J=0.5 Hz, 1H), 7.22-7.18 (m, 1H), 6.24 (dd, J=5.8 Hz, 2.2 Hz, 1H), 5.94 (d, J=2.2 Hz, 1H), 4.38 (d, J=5.7 Hz, 2H), 3.91 (s, 3H), 3.77 (s, 3H), 2.13 (s, 3H).

ESI-MS: 463.20 (M+H)⁺.

### N-[(3-fluorophenyl)methyl]-2-methyl-3-({2-[(2-methylpyrazol-3-yl)amino]-4-pyridyl}oxy)benzamide (184)

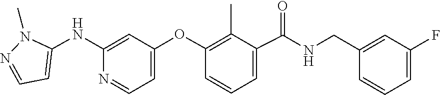 [SMILES: Cc1c(Oc2ccnc(Nc3ccnn3C)c2)cccc1C(=O)NCc1cccc(F)c1]

Compound 184 was synthesized from intermediate 119g (0.15 mmol) and 3-fluorobenzylamine (0.23 mmol) as a white solid in 18% yield according to the general method C2. ¹H NMR (400 MHz, DMSO-d₆) δ (ppm): 8.95 (t, J=6.1 Hz, 1H), 8.75 (s, 1H), 8.00 (d, J=5.8 Hz, 1H), 7.43-7.32 (m, 3H), 7.27 (d, J=1.9 Hz, 1H), 7.23-7.13 (m, 3H), 7.09 (td, J=8.4 Hz, 2.4 Hz, 1H), 6.36 (dd, J=5.8 Hz, 2.2 Hz, 1H), 6.16 (d, J=1.9 Hz, 1H), 6.12 (d, J=2.2 Hz, 1H), 4.47 (d, J=6.0 Hz, 2H), 3.61 (s, 3H), 2.13 (s, 3H).

ESI-MS: 432.15 (M+H)⁺.

### N-[(4-fluorophenyl)methyl]-2-methyl-3-({2-[(2-methylpyrazol-3-yl)amino]-4-pyridyl}oxy)benzamide (185)

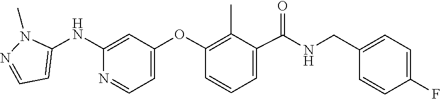 [SMILES: Cc1c(Oc2ccnc(Nc3ccnn3C)c2)cccc1C(=O)NCc1ccc(F)cc1]

Compound 185 was synthesized from intermediate 119g (0.15 mmol) and 4-fluorobenzylamine (0.23 mmol) as a white solid in 21% yield according to the general method C2. ¹H NMR (400 MHz, DMSO-d₆) δ (ppm): 8.92 (t, J=6.0 Hz, 1H), 8.75 (s, 1H), 8.00 (d, J=5.8 Hz, 1H), 7.40-7.34 (m, 3H), 7.31 (dd, J=7.6 Hz, 1.3 Hz, 1H), 7.27 (d, J=1.9 Hz, 1H), 7.22-7.14 (m, 3H), 6.36 (dd, J=5.8 Hz, 2.2 Hz, 1H), 6.16 (d, J=1.9 Hz, 1H), 6.11 (d, J=2.2 Hz, 1H), 4.43 (d, J=6.0 Hz, 2H), 3.61 (s, 3H), 2.12 (s, 3H).

ESI-MS: 432.15 (M+H)⁺.

### N-[(3-chlorophenyl)methyl]-2-methyl-3-({2-[(2-methylpyrazol-3-yl)amino]-4-pyridyl}oxy)benzamide (186)

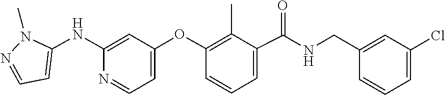 [SMILES: Cc1c(Oc2ccnc(Nc3ccnn3C)c2)cccc1C(=O)NCc1cccc(Cl)c1]

Compound 186 was synthesized from intermediate 119g (0.15 mmol) and 3-chlorobenzylamine (0.23 mmol) as a white solid in 13% yield according to the general method C2. ¹H NMR (400 MHz, DMSO-d₆) δ (ppm): 8.96 (t, J=6.0 Hz, 1H), 8.75 (s, 1H), 8.00 (d, J=5.8 Hz, 1H), 7.41-7.36 (m, 3H), 7.34-7.30 (m, 3H), 7.27 (d, J=1.9 Hz, 1H), 7.22 (dd, J=7.8 Hz, 1.2 Hz, 1H), 6.36 (dd, J=5.8 Hz, 2.2 Hz, 1H), 6.16 (d, J=1.9 Hz, 1H), 6.12 (d, J=2.2 Hz, 1H), 4.46 (d, J=6.0 Hz, 2H), 3.61 (s, 3H), 2.13 (s, 3H).

ESI-MS: 448.10 (M+H)⁺.

### N-[(4-chlorophenyl)methyl]-2-methyl-3-({2-[(2-methylpyrazol-3-yl)amino]-4-pyridyl}oxy)benzamide (187)

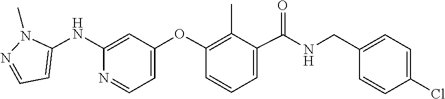 [SMILES: Cc1c(Oc2ccnc(Nc3ccnn3C)c2)cccc1C(=O)NCc1ccc(Cl)cc1]

Compound 187 was synthesized from intermediate 119g (0.15 mmol) and 4-chlorobenzylamine (0.23 mmol) as a white solid in 18% yield according to the general method C2. ¹H NMR (400 MHz, DMSO-d₆) δ (ppm): 9.17 (s, 1H), 8.94 (t, J=6.0 Hz, 1H), 8.75 (s, 1H), 8.00 (d, J=5.8 Hz, 1H), 7.43-7.30 (m, 6H), 7.27 (d, J=1.9 Hz, 1H), 7.21 (dd, J=7.8 Hz, 1.3 Hz, 1H), 6.36 (dd, J=5.8 Hz, 2.2 Hz, 1H), 6.16 (d, J=1.9 Hz, 1H), 6.11 (d, J=2.2 Hz, 1H), 4.43 (d, J=6.0 Hz, 2H), 3.61 (s, 3H), 2.12 (s, 3H).

### N-(imidazo[1,2-a]pyridin-6-ylmethyl)-2-methyl-3-({2-[(2-methylpyrazol-3-yl)amino]-4-pyridyl}oxy)benzamide (188)

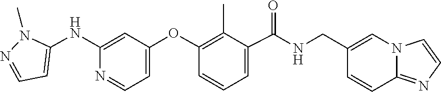 [SMILES: Cc1c(Oc2ccnc(Nc3ccnn3C)c2)cccc1C(=O)NCc1ccc2nccn2c1]

Compound 188 was synthesized from intermediate 119g (0.15 mmol) and imidazo[1,2-a]pyridin-6-ylmethanamine (0.23 mmol) as a white solid in 18% yield according to the general method C2. ¹H NMR (400 MHz, DMSO-d₆) δ (ppm): 8.94 (t, J=5.9 Hz, 1H), 8.75 (s, 1H), 8.50 (s, 1H), 7.99 (d, J=5.8 Hz, 1H), 7.96 (s, 1H), 7.57-7.54 (m, 2H), 7.39-7.32 (m, 2H), 7.27 (d, J=1.9 Hz, 1H), 7.26-7.20 (m, 2H), 6.35 (dd, J=5.8 Hz, 2.2 Hz, 1H), 6.16 (d, J=1.9 Hz, 1H), 6.11 (d, J=2.2 Hz, 1H), 4.44 (d, J=5.8 Hz, 2H), 3.61 (s, 3H), 2.13 (s, 3H).

ESI-MS: 454.15 (M+H)⁺.

### N-(imidazo[1,2-a]pyridin-7-ylmethyl)-2-methyl-3-({2-[(2-methylpyrazol-3-yl)amino]-4-pyridyl}oxy)benzamide (189)

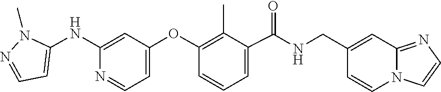 [SMILES: Cc1c(Oc2ccnc(Nc3ccnn3C)c2)cccc1C(=O)NCc1ccn2ccnc2c1]

Compound 189 was synthesized from intermediate 119g (0.15 mmol) and imidazo[1,2-a]pyridin-7-ylmethanamine (0.23 mmol) as a white solid in 13% yield according to the general method C2. ¹H NMR (400 MHz, DMSO-d₆) δ (ppm): 8.99 (t, J=6.0 Hz, 1H), 8.75 (s, 1H), 8.51 (d, J=6.9 Hz, 1H), 8.00 (d, J=5.8 Hz, 1H), 7.90 (s, 1H), 7.53 (d, J=1.1 Hz, 1H), 7.44 (s, 1H), 7.41-7.34 (m, 2H), 7.27 (d, J=1.9 Hz, 1H), 7.23 (dd, J=7.3 Hz, 1.9 Hz, 1H), 6.88 (dd, J=7.0 Hz, 1.6 Hz, 1H), 6.37 (dd, J=5.8 Hz, 2.2 Hz, 1H), 6.16 (d, J=1.9 Hz, 1H), 6.12 (d, J=2.2 Hz, 1H), 4.49 (d, J=5.9 Hz, 2H), 3.61 (s, 3H), 2.15 (s, 3H).

ESI-MS: 454.15 (M+H)⁺.

### N-[(3,5-difluorophenyl)methyl]-2-methyl-3-({2-[(2-methylpyrazol-3-yl)amino]-4-pyridyl}oxy)benzamide (190)

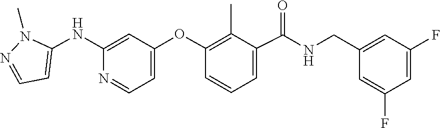 [SMILES: Cc1c(Oc2ccnc(Nc3ccnn3C)c2)cccc1C(=O)NCc1cc(F)cc(F)c1]

Compound 190 was synthesized from intermediate 119g (0.15 mmol) and 3,5-difluorobenzylamine (0.23 mmol) as a white solid in 16% yield according to the general method C2. ¹H NMR (400 MHz, DMSO-d₆) δ (ppm): 8.99 (t, J=6.0 Hz, 1H), 8.75 (s, 1H), 8.00 (d, J=5.8 Hz, 1H), 7.41-7.34 (m, 2H), 7.27 (d, J=1.9 Hz, 1H), 7.23 (dd, J=7.4 Hz, 1.8 Hz, 1H), 7.13 (tt, J=9.4 Hz, 2.3 Hz, 1H), 7.08-7.03 (m, 2H), 6.36 (dd, J=5.8 Hz, 2.2 Hz, 1H), 6.16 (d, J=1.9 Hz, 1H), 6.12 (d, J=2.1 Hz, 1H), 4.47 (d, J=6.0 Hz, 2H), 3.61 (s, 3H), 2.13 (s, 3H).

ESI-MS: 450.10 (M+H)⁺.

### N-[(6-methoxy-3-pyridyl)methyl]-2-methyl-3-({2-[(2-methylpyrazol-3-yl)amino]-4-pyridyl}oxy)benzamide (191)

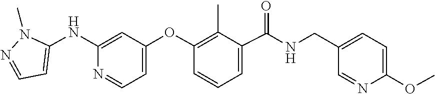 [SMILES: COc1ccc(CNC(=O)c2cccc(Oc3ccnc(Nc4ccnn4C)c3)c2C)cn1]

Compound 191 was synthesized from intermediate 119g (0.15 mmol) and (6-methoxypyridin-3-yl) methanamine (0.23 mmol) as a white solid in 15% yield according to the general method C2. ¹H NMR (400 MHz, DMSO-d₆) δ (ppm): 8.88 (t, J=6.0 Hz, 1H), 8.74 (s, 1H), 8.13 (d, J=2.0 Hz, 1H), 7.99 (d, J=5.8 Hz, 1H), 7.69 (dd, J=8.5 Hz, 2.5 Hz, 1H), 7.36 (t, J=7.7 Hz, 1H), 7.30-7.27 (m, 2H), 7.20 (dd, J=7.9 Hz, 1.1 Hz, 1H), 6.81 (d, J=8.1 Hz, 1H), 6.35 (dd, J=5.8 Hz, 2.2 Hz, 1H), 6.16 (d, J=1.9 Hz, 1H), 6.11 (d, J=2.2 Hz, 1H), 4.38 (d, J=5.9 Hz, 2H), 3.83 (s, 3H), 3.61 (s, 3H), 2.11 (s, 3H).

ESI-MS: 445.15 (M+H)⁺.

### 2-methyl-3-({2-[(2-methylpyrazol-3-yl)amino]-4-pyridyl}oxy)-N-{[6-(trifluoro methyl)-3-pyridyl]methyl} benzamide (192)

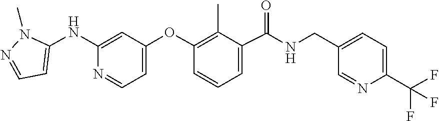 [SMILES: Cc1c(Oc2ccnc(Nc3ccnn3C)c2)cccc1C(=O)NCc1ccc(C(F)(F)F)nc1]

Compound 192 was synthesized from intermediate 119g (0.15 mmol) and [6-(trifluoromethyl)-3-pyridyl]methanamine (0.23 mmol) as a white solid in 10% yield according to the general method C2. ¹H NMR (400 MHz, DMSO-d₆) δ (ppm): 9.08 (t, J=5.9 Hz, 1H), 8.77 (s, 2H), 8.04 (d, J=9.0 Hz, 1H), 8.00 (d, J=5.8 Hz, 1H), 7.92 (d, J=8.1 Hz, 1H), 7.40-7.35 (m, 2H), 7.27 (d, J=1.8 Hz, 1H), 7.23 (dd, J=6.8 Hz, 2.5 Hz, 1H), 6.36 (dd, J=5.8 Hz, 2.2 Hz, 1H), 6.16 (d, J=1.8 Hz, 1H), 6.11 (d, J=2.1 Hz, 1H), 4.58 (d, J=5.8 Hz, 2H), 3.61 (s, 3H), 2.13 (s, 3H).

ESI-MS: 483.15 (M+H)⁺.

### N-[(5-fluoro-3-pyridyl)methyl]-2-methyl-3-({2-[(2-methylpyrazol-3-yl)amino]-4-pyridyl}oxy)benzamide (193)

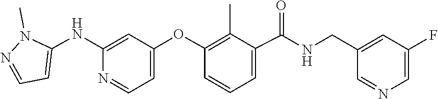 [SMILES: Cc1c(Oc2ccnc(Nc3ccnn3C)c2)cccc1C(=O)NCc1cncc(F)c1]

Compound 193 was synthesized from intermediate 119g (0.09 mmol) and 5-fluoro-3-pyridinemethanamine (0.14 mmol) as a white solid in 13% yield according to the general method C2. ¹H NMR (400 MHz, DMSO-d₆) δ (ppm): 9.01 (t, J=5.9 Hz, 1H), 8.75 (s, 1H), 8.49 (d, J=2.8 Hz, 1H), 8.47 (s, 1H), 8.00 (d, J=5.8 Hz, 1H), 7.69-7.65 (m, 1H), 7.40-7.34 (m, 2H), 7.27 (d, J=1.9 Hz, 1H), 7.22 (dd, J=7.2 Hz, 2.1 Hz, 1H), 6.36 (dd, J=5.8 Hz, 2.2 Hz, 1H), 6.16 (d, J=1.9 Hz, 1H), 6.11 (d, J=2.2 Hz, 1H), 4.52 (d, J=5.9 Hz, 2H), 3.61 (s, 3H), 2.12 (s, 3H).

ESI-MS: 433.20 (M+H)⁺.

### N-[(5-fluoro-6-methoxy-3-pyridyl)methyl]-2-methyl-3-({2-[(2-methylpyrazol-3-yl)amino]-4-pyridyl}oxy)benzamide (194)

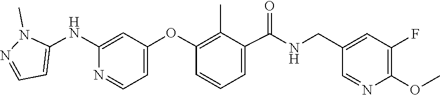 [SMILES: COc1ncc(CNC(=O)c2cccc(Oc3ccnc(Nc4ccnn4C)c3)c2C)cc1F]

Compound 194 was synthesized from intermediate 119g (0.09 mmol) and (5-fluoro-6-methoxy-3-pyridyl) methanamine (0.14 mmol) as a white solid in 19% yield according to the general method C2. ¹H NMR (400 MHz, DMSO-d₆) δ (ppm): 8.91 (t, J=5.9 Hz, 1H), 8.75 (s, 1H), 8.00 (d, J=5.8 Hz, 1H), 7.97 (d, J=1.7 Hz, 1H), 7.64 (dd, J=11.4 Hz, 1.9 Hz, 1H), 7.38-7.30 (m, 2H), 7.27 (d, J=1.9 Hz, 1H), 7.21 (dd, J=7.8 Hz, 1.3 Hz, 1H), 6.35 (dd, J=5.8 Hz, 2.2 Hz, 1H), 6.16 (d, J=1.9 Hz, 1H), 6.11 (d, J=2.2 Hz, 1H), 4.41 (d, J=5.8 Hz, 2H), 3.93 (s, 3H), 3.61 (s, 3H), 2.11 (s, 3H).

ESI-MS: 463.25 (M+H)⁺.

### N-[(5-fluoro-2-methoxy-3-pyridyl)methyl]-2-methyl-3-({2-[(2-methylpyrazol-3-yl)amino]-4-pyridyl}oxy)benzamide (195)

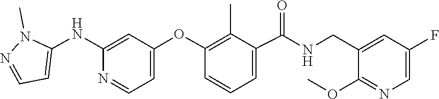 [SMILES: COc1ncc(F)cc1CNC(=O)c1cccc(Oc2ccnc(Nc3ccnn3C)c2)c1C]

Compound 195 was synthesized from intermediate 119g (0.09 mmol) and 5-fluoro-2-methoxy-3-pyridinemethanamine (0.14 mmol) as a white solid in 14% yield according to the general method C2. ¹H NMR (400 MHz, DMSO-d₆) δ (ppm): 8.85 (t, J=5.7 Hz, 1H), 8.75 (s, 1H), 8.07 (d, J=3.0 Hz, 1H), 8.00 (d, J=5.8 Hz, 1H), 7.55 (dd, J=8.6 Hz, 3.0 Hz, 1H), 7.40-7.35 (m, 2H), 7.27 (d, J=1.9 Hz, 1H), 7.25-7.20 (m, 1H), 6.36 (dd, J=5.8 Hz, 2.2 Hz, 1H), 6.16 (d, J=1.9 Hz, 1H), 6.12 (d, J=2.2 Hz, 1H), 4.38 (d, J=5.7 Hz, 2H), 3.91 (s, 3H), 3.61 (s, 3H), 2.14 (s, 3H).

ESI-MS: 463.25 (M+H)⁺.

### N-[(5-fluoro-6-methoxy-3-pyridyl)methyl]-2-methyl-3-({2-[(2-methyltriazol-4-yl)amino]-4-pyridyl}oxy)benzamide (350)

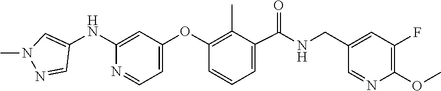

Compound 350 was synthesized from intermediate 119l (0.08 mmol) and (5-fluoro-6-methoxy-3-pyridyl) methanamine (0.12 mmol) as a white solid in 56% yield according to the general method C2. ¹H NMR (400 MHz, DMSO-d₆) δ (ppm): 9.59 (s, 1H), 8.90 (t, J=5.8 Hz, 1H), 8.06 (d, J=5.9 Hz, 1H), 7.97 (s, 1H), 7.80 (s, 1H), 7.68-7.61 (m, 1H), 7.38-7.30 (m, 2H), 7.21-7.18 (m, 1H), 6.47 (d, J=2.2 Hz, 1H), 6.30 (dd, J=5.8 Hz, 2.3 Hz, 1H), 4.41 (d, J=5.6 Hz, 2H), 4.00 (s, 3H), 3.93 (s, 3H), 2.11 (s, 3H).

ESI-MS: 464.05 (M+H)⁺.

### N-[(6-methoxy-3-pyridyl)methyl]-2-methyl-3-[(2-pyrazol-1-yl-4-pyridyl)oxy] benzamide (196)

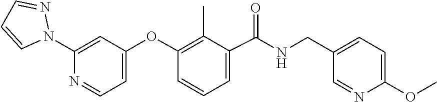 [SMILES: COc1ccc(CNC(=O)c2cccc(Oc3ccnc(-n4cccn4)c3)c2C)cn1]

Compound 196 was synthesized from intermediate 119h (0.09 mmol) and 6-methoxypyridin-3-yl) methanamine (0.14 mmol) as a white solid in 53% yield according to the general method C2. ¹H NMR (400 MHz, DMSO-d₆) δ (ppm): 9.00 (t, J=5.9 Hz, 1H), 8.59 (d, J=2.5 Hz, 1H), 8.37 (d, J=5.7 Hz, 1H), 8.14 (d, J=2.2 Hz, 1H), 7.75 (d, J=1.1 Hz, 1H), 7.69 (dd, J=8.5 Hz, 2.4 Hz, 1H), 7.41 (t, J=7.7 Hz, 1H), 7.36-7.29 (m, 2H), 7.18 (d, J=2.3 Hz, 1H), 6.93 (dd, J=5.7 Hz, 2.3 Hz, 1H), 6.81 (d, J=8.5 Hz, 1H), 6.55-6.54 (m, 1H), 4.38 (d, J=5.9 Hz, 2H), 3.82 (s, 3H), 2.11 (s, 3H).

ESI-MS: 416.05 (M+H)⁺.

### N-[(3,5-difluorophenyl)methyl]-2-methyl-3-[(2-pyrazol-1-yl-4-pyridyl)oxy] benzamide (197)

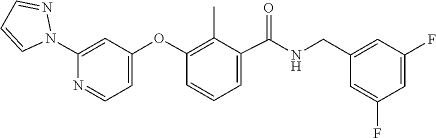 [SMILES: Cc1c(Oc2ccnc(-n3cccn3)c2)cccc1C(=O)NCc1cc(F)cc(F)c1]

Compound 197 was synthesized from intermediate 119h (0.09 mmol) and 3,5-difluorobenzylamine (0.14 mmol) as a white solid in 74% yield according to the general method D. ¹H NMR (400 MHz, DMSO-d₆) δ (ppm): 9.09 (s, 1H), 8.59 (d, J=1.9 Hz, 1H), 8.37 (d, J=5.6 Hz, 1H), 7.75 (s, 1H), 7.46-7.32 (m, 3H), 7.19-7.06 (m, 4H), 6.95-6.93 (m, 1H), 6.55 (s, 1H), 4.47 (d, J=5.8 Hz, 2H), 2.14 (s, 3H).

ESI-MS: 421.05 (M+H)⁺.

### Example 7: General Procedure for the Synthesis of Analogues 200-247

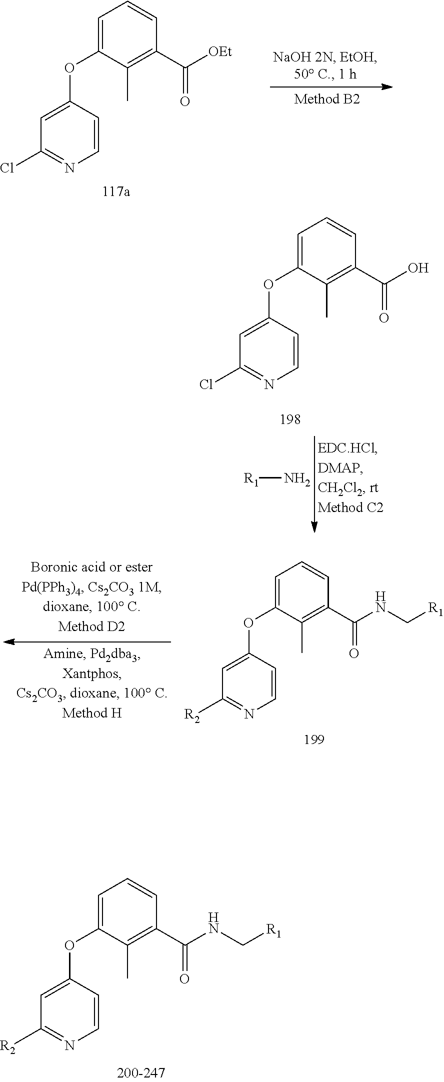

### Preparation of 3-[(2-chloro-4-pyridyl)oxy]-2-methyl-benzoic acid (198)

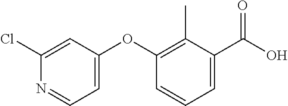 [SMILES: Cc1c(Oc2ccnc(Cl)c2)cccc1C(=O)O]

Intermediate 198 was synthesized from 117a (1.37 mmol) as a white solid in quantitative yield according to the general method B2.

The following table illustrates intermediates 199 prepared from Method C2.

| | | Synthesis |
|---|---|---|
| Intermediate | Structure | procedure |
| Compound 199a | 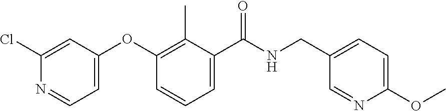 [SMILES: COc1ccc(CNC(=O)c2cccc(Oc3ccnc(Cl)c3)c2C)cn1] | Method C2 |
| Compound 199b | 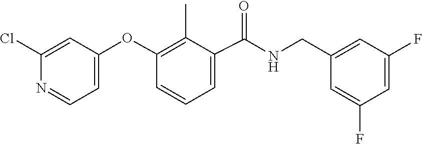 [SMILES: Cc1c(Oc2ccnc(Cl)c2)cccc1C(=O)NCc1cc(F)cc(F)c1] | Method C2 |

The following compounds are examples illustrating Method D2:

### N-[(6-methoxy-3-pyridyl)methyl]-2-methyl-3-{[2-(4-pyridyl)-4-pyridyl]oxy} benzamide (200)

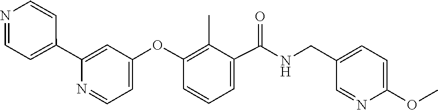 [SMILES: COc1ccc(CNC(=O)c2cccc(Oc3ccnc(-c4ccncc4)c3)c2C)cn1]

Compound 200 was synthesized from intermediate 199a (0.07 mmol) and pyridine-4-boronic acid hydrate (0.10 mmol) as a white solid in 61% yield according to the general method D2. ¹H NMR (400 MHz, DMSO-d₆) δ (ppm): 8.89 (t, J=5.9 Hz, 1H), 8.70-8.68 (m, 2H), 8.59 (d, J=5.6 Hz, 1H), 8.14 (d, J=2.0 Hz, 1H), 8.02-8.00 (m, 2H), 7.71-7.68 (m, 2H), 7.39 (t, J=7.8 Hz, 1H), 7.32 (dd, J=7.6 Hz, 1.3 Hz, 1H), 7.25 (dd, J=7.9 Hz, 1.2 Hz, 1H), 6.81 (d, J=8.5 Hz, 1H), 6.76 (dd, J=5.6 Hz, 2.4 Hz, 1H), 4.39 (d, J=5.9 Hz, 2H), 3.83 (s, 3H), 2.13 (s, 3H).

ESI-MS: 427.10 (M+H)⁺.

### N-[(6-methoxy-3-pyridyl)methyl]-2-methyl-3-{[2-(3-pyridyl)-4-pyridyl]oxy} benzamide (201)

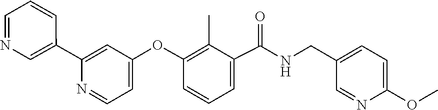 [SMILES: COc1ccc(CNC(=O)c2cccc(Oc3ccnc(-c4cccnc4)c3)c2C)cn1]

Compound 201 was synthesized from intermediate 199a (0.07 mmol) and pyridine-3-boronic acid (0.10 mmol) as a white solid in 61% yield according to the general method D2. ¹H NMR (400 MHz, DMSO-d₆) δ (ppm): 9.21 (d, J=1.6 Hz, 1H), 8.89 (t, J=5.9 Hz, 1H), 8.64 (dd, J=4.8 Hz, 1.6 Hz, 1H), 8.56 (d, J=5.7 Hz, 1H), 8.40-8.37 (m, 1H), 8.14 (d, J=2.0 Hz, 1H), 7.70 (dd, J=8.5 Hz, 2.5 Hz, 1H), 7.63 (d, J=Hz 2.2, 1H), 7.51 (ddd, J=8.0 Hz, 4.8 Hz, 0.7 Hz, 1H), 7.38 (t, J=7.8 Hz, 1H), 7.32 (dd, J=7.6 Hz, 1.3 Hz, 1H), 7.25 (dd, J=7.9 Hz, 1.2 Hz, 1H), 6.81 (d, J=8.5 Hz, 1H), 6.71 (dd, J=5.7 Hz, 2.4 Hz, 1H), 4.39 (d, J=5.9 Hz, 2H), 3.83 (s, 3H), 2.13 (s, 3H).

ESI-MS: 427.10 (M+H)⁺.

### N-[(6-methoxy-3-pyridyl)methyl]-2-methyl-3-[(2-pyrimidin-5-yl-4-pyridyl)oxy] benzamide (202)

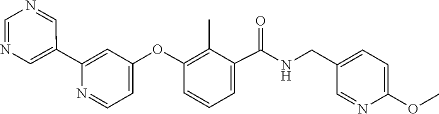 [SMILES: COc1ccc(CNC(=O)c2cccc(Oc3ccnc(-c4cncnc4)c3)c2C)cn1]

Compound 202 was synthesized from intermediate 199a (0.07 mmol) and pyrimidine-5-boronic acid (0.10 mmol) as a white solid in 72% yield according to the general method D2. ¹H NMR (400 MHz, DMSO-d₆) δ (ppm): 9.41 (s, 2H), 9.26 (s, 1H), 8.89 (t, J=5.9 Hz, 1H), 8.59 (d, J=5.7 Hz, 1H), 8.14 (d, J=2.0 Hz, 1H), 7.80 (d, J=2.2 Hz, 1H), 7.70 (dd, J=8.5 Hz, 2.5 Hz, 1H), 7.39 (t, J=7.8 Hz, 1H), 7.32 (dd, J=7.6 Hz, 1.3 Hz, 1H), 7.25 (dd, J=7.9 Hz, 1.2 Hz, 1H), 6.81 (d, J=8.5 Hz, 1H), 6.72 (dd, J=5.7 Hz, 2.4 Hz, 1H), 4.39 (d, J=5.9 Hz, 2H), 3.83 (s, 3H), 2.13 (s, 3H).

ESI-MS: 428.05 (M+H)⁺.

### N-[(6-methoxy-3-pyridyl)methyl]-2-methyl-3-{[2-(2-methylpyrazol-3-yl)-4-pyridyl]oxy}benzamide (203)

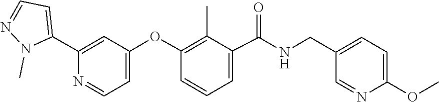 [SMILES: COc1ccc(CNC(=O)c2cccc(Oc3ccnc(-c4ccnn4C)c3)c2C)cn1]

Compound 203 was synthesized from intermediate 199a (0.07 mmol) and 1-methyl-1H-pyrazole-5-boronic acid pinacol ester (0.10 mmol) as a white solid in 43% yield according to the general method D2. ¹H NMR (400 MHz, DMSO-d₆) δ (ppm): 8.89 (t, J=5.9 Hz, 1H), 8.53 (d, J=5.7 Hz, 1H), 8.14 (d, J=2.0 Hz, 1H), 7.69 (dd, J=8.5 Hz, 2.5 Hz, 1H), 7.46 (d, J=2.0 Hz, 1H), 7.38 (t, J=7.8 Hz, 1H), 7.32-7.30 (m, 2H), 7.24 (dd, J=7.9 Hz, 1.2 Hz, 1H), 6.81 (d, J=8.5 Hz, 1H), 6.73 (d, J=2.0 Hz, 1H), 6.71 (dd, J=5.7 Hz, 2.5 Hz, 1H), 4.39 (d, J=5.9 Hz, 2H), 4.11 (s, 3H), 3.83 (s, 3H), 2.12 (s, 3H).

ESI-MS: 430.10 (M+H)⁺.

### 3-{[2-(3,5-dimethylisoxazol-4-yl)-4-pyridyl]oxy}-N-[(6-methoxy-3-pyridyl)methyl]-2-methyl-benzamide (204)

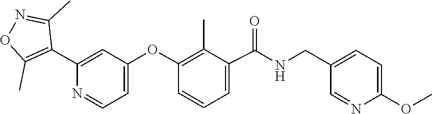 [SMILES: COc1ccc(CNC(=O)c2cccc(Oc3ccnc(-c4c(C)noc4C)c3)c2C)cn1]

Compound 204 was synthesized from intermediate 199a (0.07 mmol) and 3,5-dimethylisoxazole-4-boronic acid pinacol ester (0.10 mmol) as a white solid in 35% yield according to the general method D2. ¹H NMR (400 MHz, DMSO-d₆) δ (ppm): 8.88 (t, J=5.9 Hz, 1H), 8.52 (d, J=5.7 Hz, 1H), 8.14 (d, J=1.9 Hz, 1H), 7.69 (dd, J=8.5 Hz, 2.5 Hz, 1H), 7.38 (t, J=7.8 Hz, 1H), 7.31 (dd, J=7.6 Hz, 1.3 Hz, 1H), 7.25 (dd, J=7.9 Hz, 1.2 Hz, 1H), 6.99 (d, J=2.2 Hz, 1H), 6.81 (d, J=8.5 Hz, 1H), 6.73 (dd, J=5.7 Hz, 2.4 Hz, 1H), 4.39 (d, J=5.9 Hz, 2H), 3.83 (s, 3H), 2.31 (s, 3H), 2.12 (s, 3H).

ESI-MS: 445.10 (M+H)⁺.

### 3-{[2-(1,3-dimethylpyrazol-4-yl)-4-pyridyl]oxy]-N-[(6-methoxy-3-pyridyl)methyl]-2-methyl-benzamide (205)

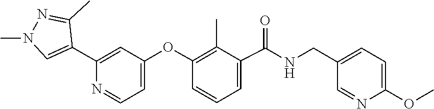

Compound 205 was synthesized from intermediate 199a (0.07 mmol) and 1,3-dimethyl-1H-pyrazole-4-boronic acid pinacol ester (0.10 mmol) as a white solid in 86% yield according to the general method D2. ¹H NMR (400 MHz, DMSO-d₆) δ (ppm): 8.88 (t, J=5.9 Hz, 1H), 8.39 (d, J=5.7 Hz, 1H), 8.14 (d, J=2.0 Hz, 1H), 8.10 (s, 1H), 7.69 (dd, J=8.5 Hz, 2.5 Hz, 1H), 7.36 (t, J=7.8 Hz, 1H), 7.30 (dd, J=7.6 Hz, 1.3 Hz, 1H), 7.21 (dd, J=7.9 Hz, 1.2 Hz, 1H), 7.01 (d, J=2.3 Hz, 1H), 6.81 (d, J=8.5 Hz, 1H), 6.54 (dd, J=5.7 Hz, 2.4 Hz, 1H), 4.39 (d, J=5.9 Hz, 2H), 3.83 (s, 3H), 3.77 (s, 3H), 2.35 (s, 3H), 2.11 (s, 3H).

ESI-MS: 444.15 (M+H)⁺.

### N-[(3,5-difluorophenyl)methyl]-2-methyl-3-{[2-(4-pyridyl)-4-pyridyl]oxy} benzamide (206)

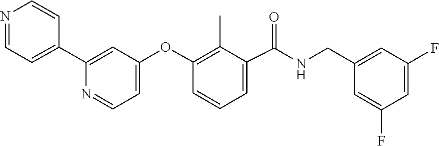 [SMILES: Cc1c(Oc2ccnc(-c3ccncc3)c2)cccc1C(=O)NCc1cc(F)cc(F)c1]

Compound 206 was synthesized from intermediate 199b (0.08 mmol) and pyridine-4-boronic acid hydrate (0.12 mmol) as a white solid in 67% yield according to the general method D2. ¹H NMR (400 MHz, DMSO-d₆) δ (ppm): 9.03 (t, J=6.0 Hz, 1H), 8.70-8.68 (m, 2H), 8.60 (d, J=5.6 Hz, 1H), 8.03-8.01 (m, 2H), 7.73 (d, J=2.3 Hz, 1H), 7.44-7.38 (m, 2H), 7.29 (dd, J=7.1 Hz, 2.2 Hz, 1H), 7.13 (tt, J=9.4 Hz, 2.3 Hz, 1H), 7.09-7.04 (m, 2H), 6.77 (dd, J=5.6 Hz, 2.4 Hz, 1H), 4.48 (d, J=6.0 Hz, 2H), 2.15 (s, 3H).

ESI-MS: 432.20 (M+H)⁺.

### N-[(3,5-difluorophenyl)methyl]-2-methyl-3-{[2-(3-pyridyl)-4-pyridyl]oxy} benzamide (207)

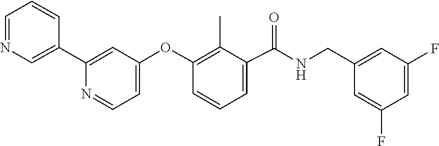 [SMILES: Cc1c(Oc2ccnc(-c3cccnc3)c2)cccc1C(=O)NCc1cc(F)cc(F)c1]

Compound 207 was synthesized from intermediate 199b (0.07 mmol) and pyridine-3-boronic acid (0.12 mmol) as a white solid in 85% yield according to the general method D2. ¹H NMR (500 MHz, DMSO-d₆) δ (ppm): 9.22 (d, J=1.7 Hz, 1H), 9.04 (t, J=6.0 Hz, 1H), 8.64 (dd, J=4.8 Hz, 1.6 Hz, 1H), 8.57 (d, J=5.7 Hz, 1H), 8.41-8.38 (m, 1H), 7.66 (d, J=2.3 Hz, 1H), 7.51 (ddd, J=8.0 Hz, 4.8 Hz, 0.7 Hz, 1H), 7.43-7.37 (m, 2H), 7.28 (dd, J=7.5 Hz, 1.8 Hz, 1H), 7.14 (tt, J=9.4 Hz, 2.4 Hz, 1H), 7.09-7.05 (m, 2H), 6.71 (dd, J=5.7 Hz, 2.4 Hz, 1H), 4.48 (d, J=6.0 Hz, 2H), 2.15 (s, 3H).

ESI-MS: 432.05 (M+H)⁺.

### N-[(3,5-difluorophenyl)methyl]-2-methyl-3-[(2-pyrimidin-5-yl-4-pyridyl)oxy] benzamide (208)

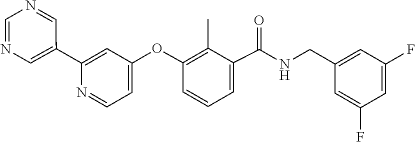 [SMILES: Cc1c(Oc2ccnc(-c3cncnc3)c2)cccc1C(=O)NCc1cc(F)cc(F)c1]

Compound 208 was synthesized from intermediate 199b (0.08 mmol) and pyrimidine-5-boronic acid (0.12 mmol) as a white solid in 85% yield according to the general method D2. ¹H NMR (500 MHz, DMSO-d₆) δ (ppm): 9.43 (s, 2H), 9.26 (s, 1H), 9.03 (t, J=6.0 Hz, 1H), 8.60 (d, J=5.7 Hz, 1H), 7.84 (d, J=2.2 Hz, 1H), 7.43-7.38 (m, 2H), 7.28 (dd, J=7.3 Hz, 2.0 Hz, 1H), 7.16-7.11 (m, 1H), 7.09-7.05 (m, 2H), 6.73 (dd, J=5.7 Hz, 2.4 Hz, 1H), 4.48 (d, J=6.0 Hz, 2H), 2.15 (s, 3H).

ESI-MS: 433.05 (M+H)⁺.

### N-[(3,5-difluorophenyl)methyl]-2-methyl-3-{[2-(2-methylpyrazol-3-yl)-4-pyridyl] oxy}benzamide (209)

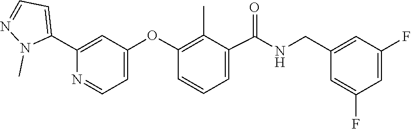 [SMILES: Cc1c(Oc2ccnc(-c3ccnn3C)c2)cccc1C(=O)NCc1cc(F)cc(F)c1]

Compound 209 was synthesized from intermediate 199b (0.08 mmol) and 1-methyl-1H-pyrazole-5-boronic acid pinacol ester (0.12 mmol) as a white solid in 45% yield according to the general method D2. ¹H NMR (500 MHz, DMSO-d₆) (ppm): 9.04 (t, J=6.1 Hz, 1H), 8.54 (d, J=5.8 Hz, 1H), 7.47 (d, J=2.0 Hz, 1H), 7.42-7.37 (m, 2H), 7.34 (d, J=2.4 Hz, 1H), 7.27 (dd, J=7.5 Hz, 1.8 Hz, 1H), 7.16-7.11 (m, 1H), 7.09-7.04 (m, 2H), 6.75 (d, J=2.0 Hz, 1H), 6.71 (dd, J=5.7 Hz, 2.5 Hz, 1H), 4.47 (d, J=6.0 Hz, 2H), 4.12 (s, 3H), 2.14 (s, 3H).

ESI-MS: 435.15 (M+H)⁺.

### N-[(3,5-difluorophenyl)methyl]-3-{[2-(3,5-dimethylisoxazol-4-yl)-4-pyridyl]oxy}-2-methyl-benzamide (210)

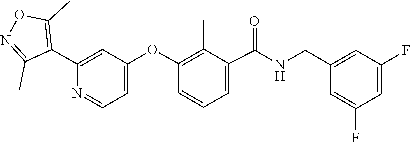 [SMILES: Cc1noc(C)c1-c1cc(Oc2cccc(C(=O)NCc3cc(F)cc(F)c3)c2C)ccn1]

Compound 210 was synthesized from intermediate 199b (0.08 mmol) and 3,5-dimethylisoxazole-4-boronic acid pinacol ester (0.12 mmol) as a white solid in 35% yield according to the general method D2. ¹H NMR (500 MHz, DMSO-d₆) δ (ppm): 9.02 (t, J=6.0 Hz, 1H), 8.53 (d, J=5.7 Hz, 1H), 7.42-7.37 (m, 2H), 7.29 (dd, J=7.4 Hz, 1.9 Hz, 1H), 7.14-7.11 (m, 1H), 7.08-7.04 (m, 2H), 7.00 (d, J=2.3 Hz, 1H), 6.74 (dd, J=5.7 Hz, 2.4 Hz, 1H), 4.47 (d, J=6.0 Hz, 2H), 2.31 (s, 3H), 2.14 (s, 3H).

ESI-MS: 450.20 (M+H)⁺.

### N-[(3,5-difluorophenyl)methyl]-3-{[2-(1,3-dimethylpyrazol-4-yl)-4-pyridyl]oxy}-2-methyl-benzamide (211)

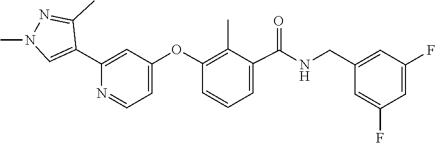 [SMILES: Cc1nn(C)cc1-c1cc(Oc2cccc(C(=O)NCc3cc(F)cc(F)c3)c2C)ccn1]

Compound 211 was synthesized from intermediate 199b (0.08 mmol) and 1,3-dimethyl-1H-pyrazole-4-boronic acid pinacol ester (0.12 mmol) as a white solid in 80% yield according to the general method D2. ¹H NMR (500 MHz, DMSO-d₆) δ (ppm): 9.02 (t, J=6.0 Hz, 1H), 8.40 (d, J=5.7 Hz, 1H), 8.12 (s, 1H), 7.41-7.35 (m, 2H), 7.24 (dd, J=7.5 Hz, 1.8 Hz, 1H), 7.14 (tt, J=9.4 Hz, 2.4 Hz, 1H), 7.08-7.04 (m, 2H), 7.03 (d, J=2.3 Hz, 1H), 6.55 (dd, J=5.7 Hz, 2.4 Hz, 1H), 4.47 (d, J=6.0 Hz, 2H), 3.77 (s, 3H), 2.35 (s, 3H), 2.13 (s, 3H).

ESI-MS: 449.90 (M+H)⁺.

### N-[(3,5-difluorophenyl)methyl]-2-methyl-3-{[2-(1-methylpyrazol-3-yl)-4-pyridyl] oxy}benzamide (212)

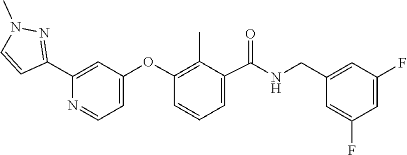 [SMILES: Cc1c(Oc2ccnc(-c3ccn(C)n3)c2)cccc1C(=O)NCc1cc(F)cc(F)c1]

Compound 212 was synthesized from intermediate 199b (0.06 mmol) and 1-methyl-3-(4,4,5,5-tetramethyl-1,3,2-dioxaborolan-2-yl)-1H-pyrazole (0.10 mmol) as a white solid in 64% yield according to the general method D2. ¹H NMR (400 MHz, DMSO-d₆) δ (ppm): 9.07 (t, J=6.0 Hz, 1H), 8.46 (d, J=5.7 Hz, 1H), 7.74 (d, J=2.2 Hz, 1H), 7.44-7.38 (m, 2H), 7.27 (dd, J=7.4 Hz, 1.8 Hz, 1H), 7.20 (d, J=2.5 Hz, 1H), 7.13 (tt, J=9.4 Hz, 2.3 Hz, 1H), 7.10-7.04 (m, 2H), 6.89 (dd, J=5.7 Hz, 2.5 Hz, 1H), 6.77 (d, J=2.2 Hz, 1H), 4.47 (d, J=6.0 Hz, 2H), 3.85 (s, 3H), 2.13 (s, 3H).

ESI-MS: 435.10 (M+H)⁺.

### N-[(3,5-difluorophenyl)methyl]-3-{[2-(1,5-dimethylpyrazol-4-yl)-4-pyridyl]oxy}-2-methyl-benzamide (213)

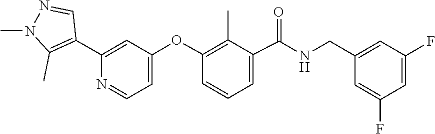 [SMILES: Cc1c(Oc2ccnc(-c3cnn(C)c3C)c2)cccc1C(=O)NCc1cc(F)cc(F)c1]

Compound 213 was synthesized from intermediate 199b (0.07 mmol) and 1,3-dimethyl-1H-pyrazole-4-boronic acid pinacol ester (0.10 mmol) as a white solid in 70% yield according to the general method D2. ¹H NMR (400 MHz, DMSO-d₆) δ (ppm): 9.00 (t, J=6.0 Hz, 1H), 8.40 (d, J=5.8 Hz, 1H), 7.81 (s, 1H), 7.41-7.35 (m, 2H), 7.23 (dd, J=7.4 Hz, 1.9 Hz, 1H), 7.16-7.04 (m, 4H), 6.51 (dd, J=5.7 Hz, 2.4 Hz, 1H), 4.48 (d, J=6.0 Hz, 2H), 3.76 (s, 3H), 2.55 (s, 3H), 2.14 (s, 3H).

ESI-MS: 449.20 (M+H)⁺.

### N-[(3,5-difluorophenyl)methyl]-2-methyl-3-{[2-(1H-pyrazol-4-yl)-4-pyridyl]oxy} benzamide (214)

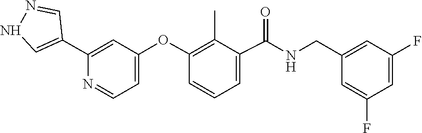 [SMILES: Cc1c(Oc2ccnc(-c3cn[nH]c3)c2)cccc1C(=O)NCc1cc(F)cc(F)c1]

Compound 214 was synthesized from intermediate 199b (0.07 mmol) and 1H-pyrazole-4-boronic acid pinacol ester (0.10 mmol) as a white solid in 41% yield according to the general method D2. ¹H NMR (400 MHz, DMSO-d₆) δ (ppm): 13.06 (bs, 1H), 8.99 (t, J=6.0 Hz, 1H), 8.36 (d, J=5.8 Hz, 1H), 8.17 (bs, 2H), 7.41-7.33 (m, 3H), 7.22 (dd, J=7.4 Hz, 1.9 Hz, 1H), 7.13 (tt, J=9.5 Hz, 2.4 Hz, 1H), 7.09-7.04 (m, 2H), 6.46 (dd, J=5.7 Hz, 2.4 Hz, 1H), 4.48 (d, J=6.0 Hz, 2H), 2.15 (s, 3H).

ESI-MS: 421.10 (M+H)⁺.

### N-[(3,5-difluorophenyl)methyl]-2-methyl-3-{[2-(1-tetrahydropyran-4-ylpyrazol-4-yl)-4-pyridyl]oxy}benzamide (215)

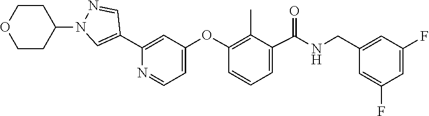 [SMILES: Cc1c(Oc2ccnc(-c3cnn(C4CCOCC4)c3)c2)cccc1C(=O)NCc1cc(F)cc(F)c1]

Compound 215 was synthesized from intermediate 199b (0.07 mmol) and 1-(tetrahydro-2H-pyran-4-yl)-4-(4,4,5,5-tetramethyl-1,3,2-dioxaborolan-2-yl)-1H-pyrazole (0.10 mmol) as a white solid in 42% yield according to the general method D2. ¹H NMR (400 MHz, DMSO-d₆) δ (ppm): 8.99 (t, J=6.0 Hz, 1H), 8.37-8.35 (m, 2H), 8.00 (s, 1H), 7.42-7.34 (m, 2H), 7.30 (d, J=2.3 Hz, 1H), 7.22 (dd, J=7.3 Hz, 1.8 Hz, 1H), 7.16-7.04 (m, 3H), 6.48 (dd, J=5.7 Hz, 2.4 Hz, 1H), 4.48-4.38 (m, 3H), 3.98-3.94 (m, 2H), 3.50-3.43 (m, 2H), 2.14 (s, 3H), 2.00-1.91 (m, 4H).

ESI-MS: 505.20 (M+H)⁺.

### 3-{[2-(1-cyclopropylpyrazol-4-yl)-4-pyridyl]oxy}-N-[(3,5-difluorophenyl)methyl]-2-methyl-benzamide (216)

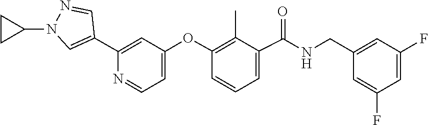 [SMILES: Cc1c(Oc2ccnc(-c3cnn(C4CC4)c3)c2)cccc1C(=O)NCc1cc(F)cc(F)c1]

Compound 216 was synthesized from intermediate 199b (0.07 mmol) and 1-cyclopropyl-4-(4,4,5,5-tetramethyl-1,3,2-dioxaborolan-2-yl)-1H-pyrazole (0.10 mmol) as a white solid in 63% yield according to the general method D2. ¹H NMR (400 MHz, DMSO-d₆) δ (ppm): 8.99 (t, J=6.0 Hz, 1H), 8.36-8.35 (m, 2H), 7.96 (d, J=0.6 Hz, 1H), 7.41-7.34 (m, 2H), 7.30 (d, J=2.4 Hz, 1H), 7.22 (dd, J=7.2 Hz, 2.1 Hz, 1H), 7.13-7.04 (m, 3H), 6.47 (dd, J=5.7 Hz, 2.4 Hz, 1H), 4.48 (d, J=6.0 Hz, 2H), 3.79-3.74 (m, 1H), 2.14 (s, 3H), 1.10-0.95 (m, 4H).

ESI-MS: 461.20 (M+H)⁺.

### 3-({2-[1-(difluoromethyl) pyrazol-4-yl]-4-pyridyl}oxy)-N-[(3,5-difluorophenyl)methyl]-2-methyl-benzamide (217)

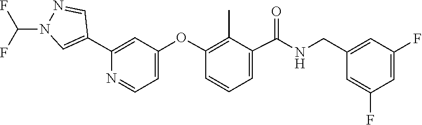 [SMILES: Cc1c(Oc2ccnc(-c3cnn(C(F)F)c3)c2)cccc1C(=O)NCc1cc(F)cc(F)c1]

Compound 217 was synthesized from intermediate 199b (0.06 mmol) and 1-(difluoromethyl)-4-(4,4,5,5-tetramethyl-1,3,2-dioxaborolan-2-yl)-1H-pyrazole (0.10 mmol) as a white solid in 100% yield according to the general method D2. ¹H NMR (400 MHz, DMSO-d₆) δ (ppm): 8.99 (t, J=6.0 Hz, 1H), 8.82 (s, 1H), 8.43 (d, J=5.7 Hz, 1H), 8.35 (s, 1H), 7.85 (t, J=59.0 Hz, 1H), 7.49 (d, J=2.3 Hz, 1H), 7.42-7.37 (m, 2H), 7.24 (dd, J=7.2 Hz, 2.2 Hz, 1H), 7.16-7.04 (m, 3H), 6.57 (dd, J=5.7 Hz, 2.4 Hz, 1H), 4.48 (d, J=6.0 Hz, 2H), 2.15 (s, 3H).

ESI-MS: 471.20 (M+H)⁺.

### N-[(3,5-difluorophenyl)methyl]-2-methyl-3-[(2-phenyl-4-pyridyl)oxy] benzamide (218)

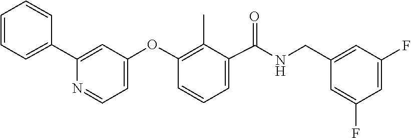 [SMILES: Cc1c(Oc2ccnc(-c3ccccc3)c2)cccc1C(=O)NCc1cc(F)cc(F)c1]

Compound 218 was synthesized from intermediate 199b (0.05 mmol) and benzene boronic acid (0.08 mmol) as a white solid in 82% yield according to the general method D2. ¹H NMR (400 MHz, DMSO-d₆) δ (ppm): 9.01 (t, J=6.0 Hz, 1H), 8.54 (d, J=5.8 Hz, 1H), 8.03-8.01 (m, 2H), 7.50-7.36 (m, 6H), 7.27 (dd, J=7.4 Hz, 1.9 Hz, 1H), 7.16-7.09 (m, 1H), 7.08-7.04 (m, 2H), 6.71 (dd, J=5.6 Hz, 2.4 Hz, 1H), 4.48 (d, J=6.0 Hz, 2H), 2.16 (s, 3H).

ESI-MS: 431.05 (M+H)⁺.

### N-[(3,5-difluorophenyl)methyl]-3-{[2-(2-fluorophenyl)-4-pyridyl]oxy}-2-methyl-benzamide (219)

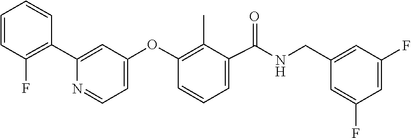 [SMILES: Cc1c(Oc2ccnc(-c3ccccc3F)c2)cccc1C(=O)NCc1cc(F)cc(F)c1]

Compound 219 was synthesized from intermediate 199b (0.05 mmol) and 2-fluorobenzeneboronic acid (0.08 mmol) as a white solid in 74% yield according to the general method D2. ¹H NMR (600 MHz, DMSO-d₆) δ (ppm): 9.00 (t, J=6.1 Hz, 1H), 8.60-8.58 (m, 1H), 7.93 (td, J=7.9 Hz, 1.8 Hz, 1H), 7.50-7.46 (m, 1H), 7.42-7.37 (m, 2H), 7.34-7.27 (m, 3H), 7.23-7.22 (m, 1H), 7.12 (tt, J=9.4 Hz, 2.4 Hz, 1H), 7.08-7.04 (m, 2H), 6.86 (dd, J=5.7 Hz, 2.4 Hz, 1H), 4.47 (d, J=6.0 Hz, 2H), 2.15 (s, 3H).

ESI-MS: 449.05 (M+H)⁺.

### N-[(3,5-difluorophenyl)methyl]-3-{[2-(3-fluorophenyl)-4-pyridyl]oxy}-2-methyl-benzamide (220)

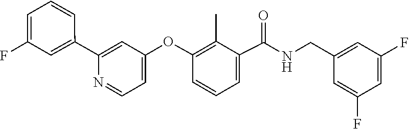 [SMILES: Cc1c(Oc2ccnc(-c3cccc(F)c3)c2)cccc1C(=O)NCc1cc(F)cc(F)c1]

Compound 220 was synthesized from intermediate 199b (0.05 mmol) and 3-fluorobenzeneboronic acid (0.08 mmol) as a white solid in 83% yield according to the general method D2. ¹H NMR (600 MHz, DMSO-d₆) δ (ppm): 9.00 (t, J=6.1 Hz, 1H), 8.55-8.54 (m, 1H), 7.90-7.85 (m, 2H), 7.61-7.60 (m, 1H), 7.54-7.51 (m, 1H), 7.42-7.37 (m, 2H), 7.30-7.26 (m, 2H), 7.12 (tt, J=9.3 Hz, 2.4 Hz, 1H), 7.08-7.05 (m, 2H), 6.71 (dd, J=5.6 Hz, 2.4 Hz, 1H), 4.48 (d, J=6.0 Hz, 2H), 2.16 (s, 3H).

ESI-MS: 449.05 (M+H)⁺.

### N-[(3,5-difluorophenyl)methyl]-3-{[2-(4-fluorophenyl)-4-pyridyl]oxy}-2-methyl-benzamide (221)

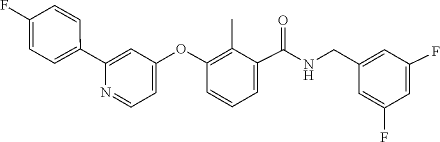 [SMILES: Cc1c(Oc2ccnc(-c3ccc(F)cc3)c2)cccc1C(=O)NCc1cc(F)cc(F)c1]

Compound 221 was synthesized from intermediate 199b (0.05 mmol) and 4-fluorobenzeneboronic acid (0.08 mmol) as a white solid in 91% yield according to the general method D2. ¹H NMR (600 MHz, DMSO-d₆) δ (ppm): 9.00 (t, J=6.0 Hz, 1H), 8.52 (d, J=5.7 Hz, 1H), 8.10-8.07 (m, 2H), 7.50 (d, J=2.3 Hz, 1H), 7.42-7.3 (m, 2H), 7.31-7.25 (m, 3H), 7.12 (tt, J=9.3 Hz, 2.4 Hz, 1H), 7.08-7.05 (m, 2H), 6.69 (dd, J=5.6 Hz, 2.4 Hz, 1H), 4.48 (d, J=6.0 Hz, 2H), 2.16 (s, 3H).

ESI-MS: 449.05 (M+H)⁺.

### N-[(3,5-difluorophenyl)methyl]-3-{[2-(2-methoxyphenyl)-4-pyridyl]oxy}-2-methyl-benzamide (222)

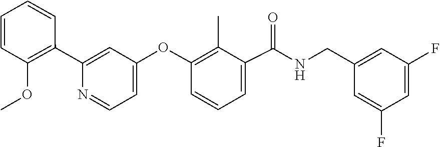 [SMILES: COc1ccccc1-c1cc(Oc2cccc(C(=O)NCc3cc(F)cc(F)c3)c2C)ccn1]

Compound 222 was synthesized from intermediate 199b (0.05 mmol) and 2-methoxybenzeneboronic acid (0.08 mmol) as a white solid in 75% yield according to the general method D2. ¹H NMR (600 MHz, DMSO-d₆) δ (ppm): 9.00 (t, J=6.1 Hz, 1H), 8.53 (dd, J=5.7 Hz, 0.4 Hz, 1H), 7.78 (dd, J=7.7 Hz, 1.8 Hz, 1H), 7.42-7.36 (m, 3H), 7.27-7.25 (m, 2H), 7.14-7.02 (m, 5H), 6.85 (dd, J=5.7 Hz, 2.5 Hz, 1H), 4.48 (d, J=6.0 Hz, 2H), 3.74 (s, 3H), 2.15 (s, 3H).

ESI-MS: 461.05 (M+H)⁺.

### N-[(3,5-difluorophenyl)methyl]-3-{[2-(3-methoxyphenyl)-4-pyridyl]oxy}-2-methyl-benzamide (223)

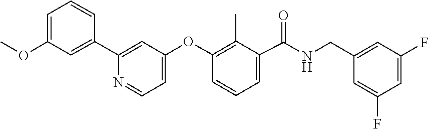 [SMILES: COc1cccc(-c2cc(Oc3cccc(C(=O)NCc4cc(F)cc(F)c4)c3C)ccn2)c1]

Compound 223 was synthesized from intermediate 199b (0.05 mmol) and 3-methoxybenzeneboronic acid (0.08 mmol) as a white solid in 84% yield according to the general method D2. ¹H NMR (600 MHz, DMSO-d₆) δ (ppm): 9.00 (t, J=6.0 Hz, 1H), 8.53 (d, J=5.6 Hz, 1H), 7.60-7.59 (m, 1H), 7.57 (dd, J=7.7 Hz, 0.9 Hz, 1H), 7.51 (d, J=2.3 Hz, 1H), 7.42-7.37 (m, 3H), 7.26 (d, J=7.7 Hz, 1H), 7.14-7.10 (m, 1H), 7.08-7.05 (m, 2H), 7.02-7.00 (m, 1H), 6.70-6.68 (m, 1H), 4.48 (d, J=6.0 Hz, 2H), 3.82 (s, 3H), 2.16 (s, 3H).

ESI-MS: 461.05 (M+H)⁺.

### N-[(3,5-difluorophenyl)methyl]-3-{[2-(4-methoxyphenyl)-4-pyridyl]oxy}-2-methyl-benzamide (224)

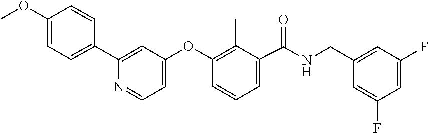 [SMILES: COc1ccc(-c2cc(Oc3cccc(C(=O)NCc4cc(F)cc(F)c4)c3C)ccn2)cc1]

Compound 224 was synthesized from intermediate 199b (0.05 mmol) and 4-methoxybenzeneboronic acid (0.08 mmol) as a white solid in 92% yield according to the general method D2. ¹H NMR (600 MHz, DMSO-d₆) δ (ppm): 9.00 (t, J=6.1 Hz, 1H), 8.48-8.47 (m, 1H), 8.00-7.97 (m, 2H), 7.41-7.36 (m, 3H), 7.26 (dd, J=7.7 Hz, 1.4 Hz, 1H), 7.12 (tt, J=9.4 Hz, 2.4 Hz, 1H), 7.08-7.05 (m, 2H), 7.03-7.00 (m, 2H), 6.63 (dd, J=5.6 Hz, 2.4 Hz, 1H), 4.48 (d, J=6.0 Hz, 2H), 3.81 (s, 3H), 2.15 (s, 3H).

ESI-MS: 461.05 (M+H)⁺.

### N-[(3,5-difluorophenyl)methyl]-2-methyl-3-{[2-(o-tolyl)-4-pyridyl]oxy}benzamide (225)

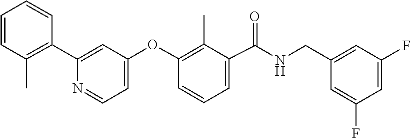 [SMILES: Cc1ccccc1-c1cc(Oc2cccc(C(=O)NCc3cc(F)cc(F)c3)c2C)ccn1]

Compound 225 was synthesized from intermediate 199b (0.05 mmol) and o-tolylboronic acid (0.08 mmol) as a white solid in 83% yield according to the general method D2. ¹H NMR (600 MHz, DMSO-d₆) δ (ppm): 8.99 (t, J=6.0 Hz, 1H), 8.54 (dd, J=5.7 Hz, 0.5 Hz, 1H), 7.41-7.23 (m, 7H), 7.12 (tt, J=9.4 Hz, 2.4 Hz, 1H), 7.07-7.04 (m, 2H), 6.86 (dd, J=2.5 Hz, 0.5 Hz, 1H), 6.84 (dd, J=5.7 Hz, 2.5 Hz, 1H), 4.47 (d, J=6.0 Hz, 2H), 2.27 (s, 3H), 2.15 (s, 3H).

ESI-MS: 445.05 (M+H)⁺.

### N-[(3,5-difluorophenyl)methyl]-2-methyl-3-{[2-(m-tolyl)-4-pyridyl]oxy}benzamide (226)

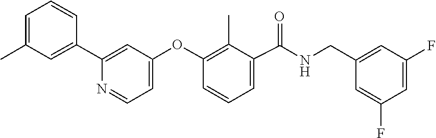 [SMILES: Cc1cccc(-c2cc(Oc3cccc(C(=O)NCc4cc(F)cc(F)c4)c3C)ccn2)c1]

Compound 226 was synthesized from intermediate 199b (0.05 mmol) and 3-tolylboronic acid (0.08 mmol) as a white solid in 83% yield according to the general method D2. ¹H NMR (600 MHz, DMSO-d₆) δ (ppm): 9.00 (t, J=6.1 Hz, 1H), 8.52 (dd, J=5.7 Hz, 0.4 Hz, 1H), 7.87 (s, 1H), 7.79 (d, J=7 Hz, 8, 1H), 7.48-7.46 (m, 1H), 7.42-7.34 (m, 3H), 7.27-7.24 (m, 2H), 7.12 (tt, J=9.4 Hz, 2.4 Hz, 1H), 7.08-7.05 (m, 2H), 6.69 (dd, J=5.6 Hz, 2.4 Hz, 1H), 4.48 (d, J=6.0 Hz, 2H), 2.38 (s, 3H), 2.16 (s, 3H).

ESI-MS: 445.05 (M+H)⁺.

### N-[(3,5-difluorophenyl)methyl]-2-methyl-3-{[2-(p-tolyl)-4-pyridyl]oxy}benzamide (227)

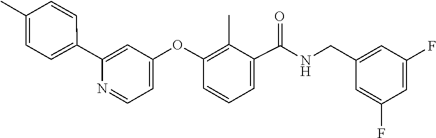 [SMILES: Cc1ccc(-c2cc(Oc3cccc(C(=O)NCc4cc(F)cc(F)c4)c3C)ccn2)cc1]

Compound 227 was synthesized from intermediate 199b (0.05 mmol) and 4-tolylboronic acid (0.08 mmol) as a white solid in 91% yield according to the general method D2. ¹H NMR (600 MHz, DMSO-d₆) δ (ppm): 9.00 (t, J=6.1 Hz, 1H), 8.51-8.50 (m, 1H), 7.92-7.91 (m, 2H), 7.44 (d, J=2.0 Hz, 1H), 7.42-7.36 (m, 2H), 7.29-7.25 (m, 3H), 7.12 (tt, J=9.4 Hz, 2.4 Hz, 1H), 7.08-7.05 (m, 2H), 6.67 (dd, J=5.6 Hz, 2.4 Hz, 1H), 4.48 (d, J=6.0 Hz, 2H), 2.35 (s, 3H), 2.15 (s, 3H).

ESI-MS: 445.05 (M+H)⁺.

The following compounds are examples illustrating Method H:

### N-[(6-methoxy-3-pyridyl)methyl]-2-methyl-3-({2-[(1-methylpyrazol-4-yl)amino]-4-pyridyl}oxy)benzamide (228)

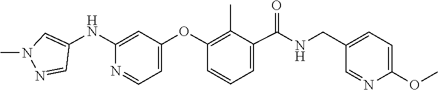

Compound 228 was synthesized from intermediate 199a (0.09 mmol) and 1-methyl-1H-pyrazol-4-ylamine (0.18 mmol) as a white solid in 54% yield according to the general method H. ¹H NMR (400 MHz, DMSO-d₆) δ (ppm): 8.89 (t, J=5.9 Hz, 1H), 8.74 (s, 1H), 8.13 (d, J=2.3 Hz, 1H), 7.98 (d, J=5.8 Hz, 1H), 7.86 (s, 1H), 7.69 (dd, J=8.5 Hz, 2.4 Hz, 1H), 7.37-7.26 (m, 3H), 7.18 (dd, J=7.9 Hz, 1.0 Hz, 1H), 6.81 (d, J=8.5 Hz, 1H), 6.23 (dd, J=5.8 Hz, 2.2 Hz, 1H), 5.93 (d, J=2.2 Hz, 1H), 4.38 (d, J=5.9 Hz, 2H), 3.83 (s, 3H), 3.76 (s, 3H), 2.10 (s, 3H).

ESI-MS: 445.25 (M+H)⁺.

### N-[(6-methoxy-3-pyridyl)methyl]-2-methyl-3-({2-[(1-methylpyrazol-3-yl)amino]-4-pyridyl}oxy)benzamide (229)

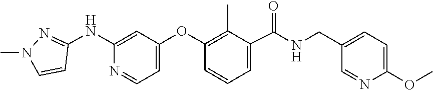

Compound 229 was synthesized from intermediate 199a (0.09 mmol) and 1-methylpyrazol-3-amine (0.18 mmol) as a white solid in 44% yield according to the general method H. ¹H NMR (400 MHz, DMSO-d₆) δ (ppm): 9.18 (s, 1H), 8.89 (t, J=5.9 Hz, 1H), 8.13 (d, J=2.3 Hz, 1H), 7.97 (d, J=5.8 Hz, 1H), 7.69 (dd, J=8.5 Hz, 2.4 Hz, 1H), 7.46 (d, J=2.2 Hz, 1H), 7.34 (t, J=7.8 Hz, 1H), 7.27 (dd, J=7.6 Hz, 1.1 Hz, 1H), 7.17 (dd, J=7.9 Hz, 1.0 Hz, 1H), 6.87 (d, J=2.0 Hz, 1H), 6.81 (d, J=8.5 Hz, 1H), 6.17 (dd, J=5.8 Hz, 2.3 Hz, 1H), 6.14 (d, J=2.2 Hz, 1H), 4.38 (d, J=5.9 Hz, 2H), 3.83 (s, 3H), 3.67 (s, 3H), 2.11 (s, 3H).

ESI-MS: 445.90 (M+H)⁺.

### N-[(3,5-difluorophenyl)methyl]-2-methyl-3-({2-[(1-methylpyrazol-4-yl)amino]-4-pyridyl}oxy)benzamide (230)

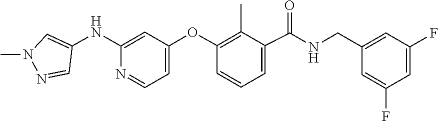 [SMILES: Cc1c(Oc2ccnc(Nc3cnn(C)c3)c2)cccc1C(=O)NCc1cc(F)cc(F)c1]

Compound 230 was synthesized from intermediate 199b (0.08 mmol) and 1-methyl-1H-pyrazol-4-ylamine (0.15 mmol) as a white solid in 72% yield according to the general method H. ¹H NMR (500 MHz, DMSO-d₆) δ (ppm): 9.01 (t, J=6.1 Hz, 1H), 8.76 (s, 1H), 7.99 (d, J=5.8 Hz, 1H), 7.87 (s, 1H), 7.39-7.33 (m, 2H), 7.31 (d, J=0.6 Hz, 1H), 7.21 (dd, J=7.6 Hz, 1.6 Hz, 1H), 7.16-7.11 (m, 1H), 7.08-7.03 (m, 2H), 6.24 (dd, J=5.8 Hz, 2.2 Hz, 1H), 5.93 (d, J=2.2 Hz, 1H), 4.47 (d, J=6.0 Hz, 2H), 3.76 (s, 3H), 2.12 (s, 3H).

ESI-MS: 450.25 (M+H)⁺.

### N-[(3,5-difluorophenyl)methyl]-2-methyl-3-({2-[(1-methylpyrazol-3-yl)amino]-4-pyridyl}oxy)benzamide (231)

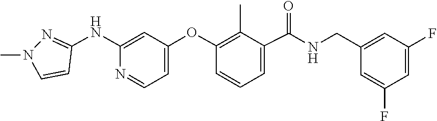 [SMILES: Cc1c(Oc2ccnc(Nc3ccn(C)n3)c2)cccc1C(=O)NCc1cc(F)cc(F)c1]

Compound 231 was synthesized from intermediate 199b (0.08 mmol) and 1-methylpyrazol-3-amine (0.15 mmol) as a white solid in 63% yield according to the general method H. ¹H NMR (500 MHz, DMSO-d₆) δ (ppm): 9.21 (s, 1H), 9.01 (t, J=6.1 Hz, 1H), 7.98 (d, J=5.8 Hz, 1H), 7.46 (d, J=2.2 Hz, 1H), 7.39-7.33 (m, 2H), 7.20 (dd, J=7.7 Hz, 1.5 Hz, 1H), 7.15-7.11 (m, 1H), 7.08-7.04 (m, 2H), 6.88 (d, J=1.6 Hz, 1H), 6.18 (dd, J=5.8 Hz, 2.3 Hz, 1H), 6.14 (d, J=2.1 Hz, 1H), 4.47 (d, J=6.0 Hz, 2H), 3.67 (s, 3H), 2.13 (s, 3H).

ESI-MS: 450.25 (M+H)⁺.

### N-[(3,5-difluorophenyl)methyl]-2-methyl-3-{[2-(2-pyridylamino)-4-pyridyl]oxy} benzamide (232)

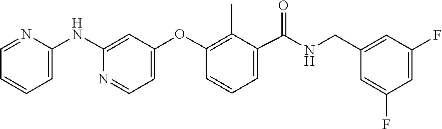 [SMILES: Cc1c(Oc2ccnc(Nc3ccccn3)c2)cccc1C(=O)NCc1cc(F)cc(F)c1]

Compound 232 was synthesized from intermediate 199b (0.10 mmol) and 2-aminopyridine (0.20 mmol) as a white solid in 33% yield according to the general method H. ¹H NMR (400 MHz, DMSO-d₆) δ (ppm): 9.70 (s, 1H), 9.01 (t, J=6.1 Hz, 1H), 8.15-8.13 (m, 1H), 8.10 (d, J=5.8 Hz, 1H), 7.65-7.59 (m, 2H), 7.43 (d, J=2.3 Hz, 1H), 7.41-7.34 (m, 2H), 7.22 (dd, J=7.3 Hz, 2.0 Hz, 1H), 7.13 (tt, J=9.3 Hz, 2.3 Hz, 1H), 7.08-7.04 (m, 2H), 6.85-6.82 (m, 1H), 6.32 (dd, J=5.8 Hz, 2.3 Hz, 1H), 4.48 (d, J=6.0 Hz, 2H), 2.15 (s, 3H).

ESI-MS: 447.15 (M+H)⁺.

### N-[(3,5-difluorophenyl)methyl]-2-methyl-3-{[2-(4-pyridylamino)-4-pyridyl]oxy} benzamide (233)

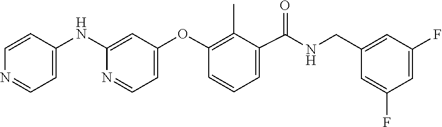 [SMILES: Cc1c(Oc2ccnc(Nc3ccncc3)c2)cccc1C(=O)NCc1cc(F)cc(F)c1]

Compound 233 was synthesized from intermediate 199b (0.10 mmol) and 4-aminopyridine (0.20 mmol) as a white solid in 44% yield according to the general method H. ¹H NMR (600 MHz, DMSO-d₆) δ (ppm): 9.48 (s, 1H), 8.98 (t, J=6.0 Hz, 1H), 8.27 (d, J=6.4 Hz, 2H), 8.17 (d, J=5.8 Hz, 1H), 7.61-7.60 (m, 2H), 7.42-7.37 (m, 2H), 7.26 (dd, J=7.5 Hz, 1.6 Hz, 1H), 7.13 (tt, J=9.4 Hz, 2.3 Hz, 1H), 7.08-7.04 (m, 2H), 6.55 (dd, J=5.8 Hz, 2.2 Hz, 1H), 6.21 (d, J=2.2 Hz, 1H), 4.48 (d, J=6.0 Hz, 2H), 2.14 (s, 3H).

ESI-MS: 447.15 (M+H)⁺.

### N-[(3,5-difluorophenyl)methyl]-2-methyl-3-{[2-(pyrimidin-2-ylamino)-4-pyridyl] oxy}benzamide (234)

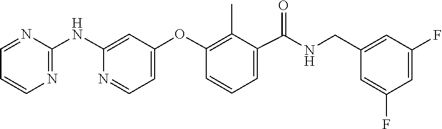 [SMILES: Cc1c(Oc2ccnc(Nc3ncccn3)c2)cccc1C(=O)NCc1cc(F)cc(F)c1]

Compound 234 was synthesized from intermediate 199b (0.08 mmol) and pyrimidin-2-amine (0.17 mmol) as a white solid in 32% yield according to the general method H. ¹H NMR (400 MHz, DMSO-d₆) δ (ppm): 9.87 (s, 1H), 9.00 (t, J=6.1 Hz, 1H), 8.51 (s, 1H), 8.50 (s, 1H), 8.14 (d, J=5.7 Hz, 1H), 7.95 (d, J=2.1 Hz, 1H), 7.41-7.34 (m, 2H), 7.23 (dd, J=7.4 Hz, 1.8 Hz, 1H), 7.13 (tt, J=9.5 Hz, 2.4 Hz, 1H), 7.09-7.04 (m, 2H), 6.93 (t, J=4.8 Hz, 1H), 6.37 (dd, J=5.7 Hz, 2.4 Hz, 1H), 4.48 (d, J=6.0 Hz, 2H), 2.15 (s, 3H).

### N-[(3,5-difluorophenyl)methyl]-2-methyl-3-{[2-(pyrimidin-4-ylamino)-4-pyridyl] oxy}benzamide (235)

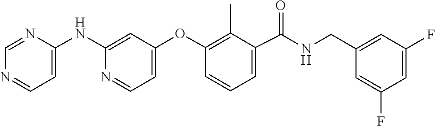 [SMILES: Cc1c(Oc2ccnc(Nc3ccncn3)c2)cccc1C(=O)NCc1cc(F)cc(F)c1]

Compound 235 was synthesized from intermediate 199b (0.08 mmol) and 4-aminopyrimidine (0.17 mmol) as a white solid in 50% yield according to the general method H. ¹H NMR (400 MHz, DMSO-d₆) δ (ppm): 10.21 (s, 1H), 9.00 (t, J=6.1 Hz, 1H), 8.65 (d, J=0.8 Hz, 1H), 8.41-8.39 (m, 1H), 8.18 (d, J=5.8 Hz, 1H), 7.70 (dd, J=5.9 Hz, 1.2 Hz, 1H), 7.42-7.34 (m, 3H), 7.24 (dd, J=7.1 Hz, 2.2 Hz, 1H), 7.12 (tt, J=9.5 Hz, 2.4 Hz, 1H), 7.09-7.03 (m, 2H), 6.47 (dd, J=5.8 Hz, 2.3 Hz, 1H), 4.48 (d, J=6.0 Hz, 2H), 2.14 (s, 3H).

ESI-MS: 448.05 (M+H)⁺.

### N-[(3,5-difluorophenyl)methyl]-2-methyl-3-{[2-(pyrimidin-5-ylamino)-4-pyridyl] oxy}benzamide (236)

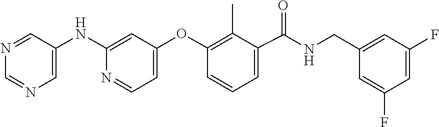 [SMILES: Cc1c(Oc2ccnc(Nc3cncnc3)c2)cccc1C(=O)NCc1cc(F)cc(F)c1]

Compound 236 was synthesized from intermediate 199b (0.08 mmol) and 5-aminopyrimidine (0.17 mmol) as a white solid in 19% yield according to the general method H. ¹H NMR (400 MHz, DMSO-d₆) δ (ppm): 9.40 (s, 1H), 9.08 (s, 2H), 8.99 (t, J=6.0 Hz, 1H), 8.69 (s, 1H), 8.13 (d, J=5.8 Hz, 1H), 7.42-7.37 (m, 2H), 7.26 (dd, J=7.3 Hz, 2.0 Hz, 1H), 7.13 (tt, J=9.4 Hz, 2.4 Hz, 1H), 7.09-7.03 (m, 2H), 6.53 (dd, J=5.8 Hz, 2.2 Hz, 1H), 6.15 (d, J=2.1 Hz, 1H), 4.48 (d, J=6.0 Hz, 2H), 2.14 (s, 3H).

### N-[(3,5-difluorophenyl)methyl]-2-methyl-3-{[2-(1H-pyrazol-3-ylamino)-4-pyridyl] oxy}benzamide (237)

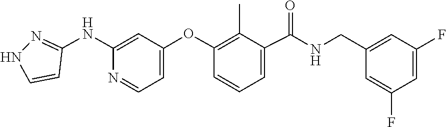 [SMILES: Cc1c(Oc2ccnc(Nc3cc[nH]n3)c2)cccc1C(=O)NCc1cc(F)cc(F)c1]

Compound 237 was synthesized from intermediate 199b (0.08 mmol) and 3-aminopyrazole (0.17 mmol) as a white solid in 17% yield according to the general method H. ¹H NMR (400 MHz, DMSO-d₆) δ (ppm): 12.00 (bs, 1H), 9.22 (s, 1H), 8.98 (t, J=6.0 Hz, 1H), 8.00 (d, J=5.8 Hz, 1H), 7.51 (d, J=1.7 Hz, 1H), 7.39-7.31 (m, 2H), 7.21-7.03 (m, 4H), 6.83 (bs, 1H), 6.18 (dd, J=5.6 Hz, 1.8 Hz, 2H), 4.47 (d, J=6.0 Hz, 2H), 2.14 (s, 3H).

ESI-MS: 436.00 (M+H)⁺.

### N-[(3,5-difluorophenyl)methyl]-2-methyl-3-({2-[(1-methyl-1,2,4-triazol-3-yl) amino]-4-pyridyl}oxy)benzamide (238)

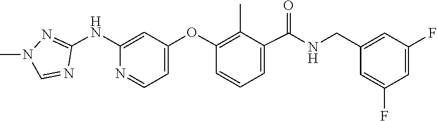 [SMILES: Cc1c(Oc2ccnc(Nc3ncn(C)n3)c2)cccc1C(=O)NCc1cc(F)cc(F)c1]

Compound 238 was synthesized from intermediate 199b (0.08 mmol) and 1-methyl-1H-1,2,4-triazol-3-amine (0.17 mmol) as a white solid in 77% yield according to the general method H. ¹H NMR (400 MHz, DMSO-d₆) δ (ppm): 9.63 (s, 1H), 8.98 (t, J=6.0 Hz, 1H), 8.20 (s, 1H), 8.04 (d, J=5.7 Hz, 1H), 7.51 (d, J=2.2 Hz, 1H), 7.40-7.33 (m, 2H), 7.20 (dd, J=7.4 Hz, 1.8 Hz, 1H), 7.12 (tt, J=9.4 Hz, 2.4 Hz, 1H), 7.10-7.03 (m, 2H), 6.21 (dd, J=5.7 Hz, 2.3 Hz, 1H), 4.47 (d, J=6.0 Hz, 2H), 3.73 (s, 3H), 2.14 (s, 3H).

ESI-MS: 451.05 (M+H)⁺.

### N-[(3,5-difluorophenyl)methyl]-2-methyl-3-({2-[(2-methyltriazol-4-yl)amino]-4-pyridyl}oxy)benzamide (239)

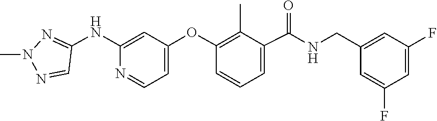 [SMILES: Cc1c(Oc2ccnc(Nc3cnn(C)n3)c2)cccc1C(=O)NCc1cc(F)cc(F)c1]

Compound 239 was synthesized from intermediate 199b (0.08 mmol) and 2-methyltriazol-4-amine (0.15 mmol) as a white solid in 57% yield according to the general method H. ¹H NMR (400 MHz, DMSO-d₆) δ (ppm): 9.61 (s, 1H), 8.99 (t, J=6.0 Hz, 1H), 8.06 (d, J=5.8 Hz, 1H), 7.81 (s, 1H), 7.41-7.34 (m, 2H), 7.21 (dd, J=7.3 Hz, 2.0 Hz, 1H), 7.13 (tt, J=9.4 Hz, 2.3 Hz, 1H), 7.08-7.03 (m, 2H), 6.48 (d, J=2.2 Hz, 1H), 6.31 (dd, J=5.8 Hz, 2.3 Hz, 1H), 4.47 (d, J=6.0 Hz, 2H), 4.00 (s, 3H), 2.14 (s, 3H).

ESI-MS: 451.10 (M+H)⁺.

### N-[(3,5-difluorophenyl)methyl]-2-methyl-3-({2-[(1-methyltriazol-4-yl)amino]-4-pyridyl}oxy)benzamide (240)

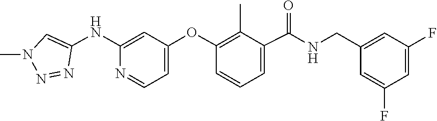 [SMILES: Cc1c(Oc2ccnc(Nc3cn(C)nn3)c2)cccc1C(=O)NCc1cc(F)cc(F)c1]

Compound 240 was synthesized from intermediate 199b (0.08 mmol) and 1-methyltriazol-4-amine (0.15 mmol) as a white solid in 30% yield according to the general method H. ¹H NMR (400 MHz, DMSO-d₆) δ (ppm): 9.63 (s, 1H), 8.98 (t, J=6.0 Hz, 1H), 8.10 (s, 1H), 8.05 (d, J=5.8 Hz, 1H), 7.40-7.34 (m, 2H), 7.20 (dd, J=7.3 Hz, 2.0 Hz, 1H), 7.13 (tt, J=9.4 Hz, 2.4 Hz, 1H), 7.09-7.03 (m, 2H), 6.36 (d, J=2.2 Hz, 1H), 6.29 (dd, J=5.8 Hz, 2.3 Hz, 1H), 4.47 (d, J=6.0 Hz, 2H), 4.00 (s, 3H), 2.14 (s, 3H).

ESI-MS: 451.10 (M+H)⁺.

### N-[(3,5-difluorophenyl)methyl]-2-methyl-3-{[2-(2-methylanilino)-4-pyridyl]oxy} benzamide (241)

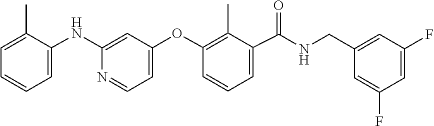 [SMILES: Cc1ccccc1Nc1cc(Oc2cccc(C(=O)NCc3cc(F)cc(F)c3)c2C)ccn1]

Compound 241 was synthesized from intermediate 199b (0.06 mmol) and o-toluidine (0.13 mmol) as a white solid in 64% yield according to the general method H. ¹H NMR (600 MHz, DMSO-d₆) δ (ppm): 8.96 (t, J=6.1 Hz, 1H), 8.15 (s, 1H), 7.94 (d, J=5.7 Hz, 1H), 7.51 (dd, J=8.0 Hz, 0.9 Hz, 1H), 7.38-7.32 (m, 2H), 7.21 (dd, J=7.8 Hz, 1.3 Hz, 1H), 7.16 (d, J=7.5 Hz, 1H), 7.14-7.08 (m, 2H), 7.07-7.03 (m, 2H), 6.95 (td, J=7.4 Hz, 1.2 Hz, 1H), 6.25 (dd, J=5.8 Hz, 2.3 Hz, 1H), 6.11 (d, J=2.2 Hz, 1H), 4.47 (d, J=6.0 Hz, 2H), 2.17 (s, 3H), 2.14 (s, 3H).

ESI-MS: 460.05 (M+H)⁺.

### N-[(3,5-difluorophenyl)methyl]-2-methyl-3-{[2-(3-methylanilino)-4-pyridyl]oxy} benzamide (242)

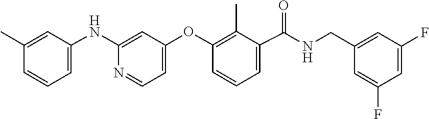 [SMILES: Cc1cccc(Nc2cc(Oc3cccc(C(=O)NCc4cc(F)cc(F)c4)c3C)ccn2)c1]

Compound 242 was synthesized from intermediate 199b (0.06 mmol) and m-toluidine (0.13 mmol) as a white solid in 74% yield according to the general method H. ¹H NMR (600 MHz, DMSO-d₆) δ (ppm): 8.97 (t, J=6.1 Hz, 1H), 8.89 (s, 1H), 8.05 (d, J=5.8 Hz, 1H), 7.41-7.35 (m, 4H), 7.23 (dd, J=7.7 Hz, 1.4 Hz, 1H), 7.14-7.04 (m, 4H), 6.69-6.67 (m, 1H), 6.37 (dd, J=5.8 Hz, 2.3 Hz, 1H), 6.12 (d, J=2.2 Hz, 1H), 4.47 (d, J=6.0 Hz, 2H), 2.24 (s, 3H), 2.15 (s, 3H).

ESI-MS: 460.10 (M+H)⁺.

### N-[(3,5-difluorophenyl)methyl]-2-methyl-3-{[2-(4-methylanilino)-4-pyridyl]oxy} benzamide (243)

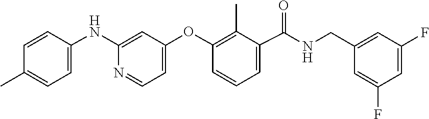 [SMILES: Cc1ccc(Nc2cc(Oc3cccc(C(=O)NCc4cc(F)cc(F)c4)c3C)ccn2)cc1]

Compound 243 was synthesized from intermediate 199b (0.06 mmol) and p-poluidine (0.13 mmol) as a white solid in 74% yield according to the general method H. ¹H NMR (600 MHz, DMSO-d₆) δ (ppm): 8.97 (t, J=6.1 Hz, 1H), 8.85 (s, 1H), 8.02 (d, J=5.8 Hz, 1H), 7.49-7.46 (m, 2H), 7.40-7.34 (m, 2H), 7.22 (dd, J=7.8 Hz, 1.4 Hz, 1H), 7.12 (tt, J=9.4 Hz, 2.4 Hz, 1H), 7.08-7.04 (m, 2H), 7.02 (d, J=8.1 Hz, 2H), 6.34 (dd, J=5.8 Hz, 2.2 Hz, 1H), 6.09 (d, J=2.2 Hz, 1H), 4.47 (d, J=6.0 Hz, 2H), 2.22 (s, 3H), 2.14 (s, 3H).

ESI-MS: 460.10 (M+H)⁺.

### N-[(3,5-difluorophenyl)methyl]-3-{[2-(2-methoxyanilino)-4-pyridyl]oxy}-2-methyl-benzamide (244)

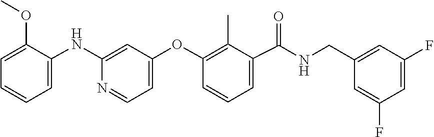 [SMILES: COc1ccccc1Nc1cc(Oc2cccc(C(=O)NCc3cc(F)cc(F)c3)c2C)ccn1]

Compound 244 was synthesized from intermediate 199b (0.06 mmol) and o-anisidine (0.13 mmol) as a white solid in 47% yield according to the general method H. ¹H NMR (400 MHz, DMSO-d₆) δ (ppm): 8.96 (t, J=6.1 Hz, 1H), 8.15 (s, 1H), 8.11 (dd, J=7.8 Hz, 1.7 Hz, 1H), 8.00 (d, J=5.8 Hz, 1H), 7.40-7.33 (m, 2H), 7.20 (dd, J=7.4 Hz, 1.9 Hz, 1H), 7.13 (tt, J=9.4 Hz, 2.3 Hz, 1H), 7.09-7.03 (m, 2H), 6.98-6.83 (m, 3H), 6.39 (d, J=2.2 Hz, 1H), 6.31 (dd, J=5.8 Hz, 2.2 Hz, 1H), 4.47 (d, J=6.0 Hz, 2H), 3.79 (s, 3H), 2.14 (s, 3H).

ESI-MS: 476.10 (M+H)⁺.

### N-[(3,5-difluorophenyl)methyl]-3-{[2-(3-methoxyanilino)-4-pyridyl]oxy}-2-methyl-benzamide (245)

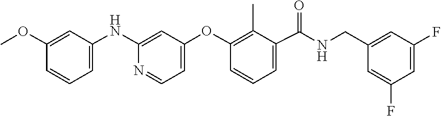 [SMILES: COc1cccc(Nc2cc(Oc3cccc(C(=O)NCc4cc(F)cc(F)c4)c3C)ccn2)c1]

Compound 245 was synthesized from intermediate 199b (0.06 mmol) and m-anisidine (0.13 mmol) as a white solid in 84% yield according to the general method H. ¹H NMR (600 MHz, DMSO-d₆) δ (ppm): 8.98-8.96 (m, 2H), 8.06 (d, J=5.8 Hz, 1H), 7.41-7.35 (m, 3H), 7.23 (dd, J=7.7 Hz, 1.4 Hz, 1H), 7.14-7.09 (m, 3H), 7.08-7.04 (m, 2H), 6.45 (dt, J=6.7 Hz, 2.4 Hz, 1H), 6.39 (dd, J=5.8 Hz, 2.3 Hz, 1H), 6.12 (d, J=2.2 Hz, 1H), 4.48 (d, J=6.0 Hz, 2H), 3.70 (s, 3H), 2.14 (s, 3H).

ESI-MS: 476.10 (M+H)⁺.

### N-[(3,5-difluorophenyl)methyl]-3-{[2-(4-methoxyanilino)-4-pyridyl]oxy}-2-methyl-benzamide (246)

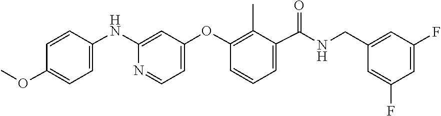 [SMILES: COc1ccc(Nc2cc(Oc3cccc(C(=O)NCc4cc(F)cc(F)c4)c3C)ccn2)cc1]

Compound 246 was synthesized from intermediate 199b (0.06 mmol) and p-anisidine (0.13 mmol) as a white solid in 81% yield according to the general method H. ¹H NMR (600 MHz, DMSO-d₆) δ (ppm): 8.97 (t, J=6.1 Hz, 1H), 8.75 (s, 1H), 7.99 (d, J=5.8 Hz, 1H), 7.59-7.46 (m, 2H), 7.40-7.34 (m, 2H), 7.22 (dd, J=7.8 Hz, 1.4 Hz, 1H), 7.12 (tt, J=9.4 Hz, 2.4 Hz, 1H), 7.08-7.04 (m, 2H), 6.84-6.81 (m, 2H), 6.30 (dd, J=5.8 Hz, 2.2 Hz, 1H), 6.04 (d, J=2.2 Hz, 1H), 4.47 (d, J=6.0 Hz, 2H), 3.69 (s, 3H), 2.14 (s, 3H).

ESI-MS: 476.10 (M+H)⁺.

### N-[(3,5-difluorophenyl)methyl]-3-({2-[(6-methoxy-2-pyridyl)amino]-4-pyridyl} oxy)-2-methyl-benzamide (247)

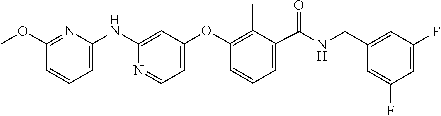 [SMILES: COc1cccc(Nc2cc(Oc3cccc(C(=O)NCc4cc(F)cc(F)c4)c3C)ccn2)n1]

Compound 247 was synthesized from intermediate 199b (0.06 mmol) and 2-amino-6-methoxypyridine (0.13 mmol) as a white solid in 68% yield according to the general method H. ¹H NMR (600 MHz, DMSO-d₆) δ (ppm): 9.65 (s, 1H), 8.95 (t, J=6.1 Hz, 1H), 8.13-8.12 (m, 1H), 7.50-7.47 (m, 2H), 7.37-7.35 (m, 2H), 7.25-7.22 (m, 1H), 7.12 (tt, J=9.4 Hz, 2.4 Hz, 1H), 7.08-7.04 (m, 2H), 6.89 (d, J=7.5 Hz, 1H), 6.56 (dd, J=5.7 Hz, 2.3 Hz, 1H), 6.18 (dd, J=7.9 Hz, 0.6 Hz, 1H), 4.47 (d, J=6.0 Hz, 2H), 3.34 (s, 3H), 2.14 (s, 3H).

ESI-MS: 477.10 (M+H)⁺.

### Example 8: General Procedure for the Synthesis of Analogues 248-261

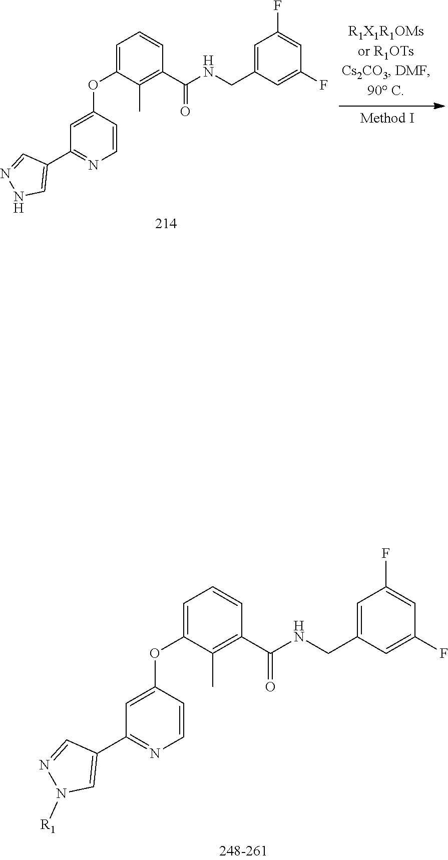

Method I: To a solution of 214 (1 equiv.) in DMF (10 mL/mmol) under nitrogen were added R₁-X derivative, R₁-OMs derivative or R₁-OTs derivative (1-2 equiv.) and Cs₂CO₃ (1.5 equiv.). The mixture was stirred at 90° C. overnight. The reaction mixture was concentrated under reduced pressure. The crude was purified by flash column chromatography (DCM/MeOH from 100/0 to 95/5) and reverse phase chromatography (H₂O/MeOH from 100/0 to 0/100) to give the expected compound.

The following compound 248 is an example illustrating Method I:

### N-[(3,5-difluorophenyl)methyl]-3-{[2-(1-isopropylpyrazol-4-yl)-4-pyridyl]oxy}-2-methyl-benzamide (248)

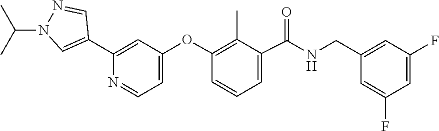 [SMILES: Cc1c(Oc2ccnc(-c3cnn(C(C)C)c3)c2)cccc1C(=O)NCc1cc(F)cc(F)c1]

Compound 248 was synthesized from intermediate 214 (0.05 mmol) and 2-iodopropane (0.05 mmol) as a white solid in 50% yield according to the general method I. ¹H NMR (400 MHz, DMSO-d₆) δ (ppm): 8.99 (t, J=6.0 Hz, 1H), 8.36 (d, J=5.7 Hz, 1H), 8.33 (s, 1H), 7.97 (s, 1H), 7.41-7.35 (m, 2H), 7.29 (d, J=2.3 Hz, 1H), 7.22 (dd, J=7.4 Hz, 1.8 Hz, 1H), 7.13 (tt, J=9.5 Hz, 2.3 Hz, 1H), 7.09-7.04 (m, 2H), 6.48 (dd, J=5.7 Hz, 2.4 Hz, 1H), 4.55-4.47 (m, 3H), 2.14 (s, 3H), 1.44 (d, J=6.7 Hz, 6H).

ESI-MS: 463.10 (M+H)⁺.

### tert-butyl 3-[4-(4-{3-[(3,5-difluorophenyl)methylcarbamoyl]-2-methyl-phenoxy}-2-pyridyl) pyrazol-1-yl]azetidine-1-carboxylate (249)

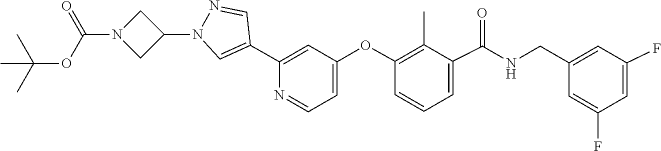 [SMILES: Cc1c(Oc2ccnc(-c3cnn(C4CN(C(=O)OC(C)(C)C)C4)c3)c2)cccc1C(=O)NCc1cc(F)cc(F)c1]

Compound 249 was synthesized from intermediate 214 (0.10 mmol) and tert-butyl 3-iodoazetidine-1-carboxylate (0.11 mmol) as a white solid in 76% yield according to the general method I. ¹H NMR (400 MHz, DMSO-d₆) δ (ppm): 8.99 (t, J=6.0 Hz, 1H), 8.46 (s, 1H), 8.38 (d, J=5.7 Hz, 1H), 8.12 (s, 1H), 7.41-7.35 (m, 2H), 7.31 (d, J=2.3 Hz, 1H), 7.23 (dd, J=7.3 Hz, 2.0 Hz, 1H), 7.13 (tt, J=9.5 Hz, 2.4 Hz, 1H), 7.09-7.04 (m, 2H), 6.51 (dd, J=5.7 Hz, 2.4 Hz, 1H), 5.27-5.20 (m, 1H), 4.48 (d, J=6.0 Hz, 2H), 4.30 (t, J=8.2 Hz, 2H), 4.15 (bs, 2H), 2.14 (s, 3H), 1.41 (s, 9H).

ESI-MS: 576.20 (M+H)⁺.

### tert-butyl 3-{[4-(4-{3-[(3,5-difluorophenyl)methylcarbamoyl]-2-methyl-phenoxy}-2-pyridyl) pyrazol-1-yl]methyl}azetidine-1-carboxylate (250)

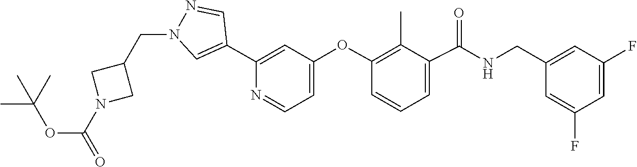 [SMILES: Cc1c(Oc2ccnc(-c3cnn(CC4CN(C(=O)OC(C)(C)C)C4)c3)c2)cccc1C(=O)NCc1cc(F)cc(F)c1]

Compound 250 was synthesized from intermediate 214 (0.10 mmol) and tert-butyl 3-(bromomethyl) azetidine-1-carboxylate (0.11 mmol) as a white solid in 63% yield according to the general method I. ¹H NMR (400 MHz, DMSO-d₆) δ (ppm): 8.99 (t, J=6.0 Hz, 1H), 8.37-8.35 (m, 2H), 8.00 (s, 1H), 7.41-7.34 (m, 2H), 7.27 (d, J=2.3 Hz, 1H), 7.22 (dd, J=7.3 Hz, 1.9 Hz, 1H), 7.13 (tt, J=9.5 Hz, 2.4 Hz, 1H), 7.10-7.04 (m, 2H), 6.49 (dd, J=5.7 Hz, 2.4 Hz, 1H), 4.48 (d, J=6.0 Hz, 2H), 4.35 (d, J=7.2 Hz, 2H), 3.88 (t, J=7.3 Hz, 2H), 3.68 (bs, 2H), 3.04-2.94 (m, 1H), 2.14 (s, 3H), 1.36 (s, 9H).

ESI-MS: 590.30 (M+H)⁺.

### N-[(3,5-difluorophenyl)methyl]-2-methyl-3-({2-[1-(oxetan-3-yl) pyrazol-4-yl]-4-pyridyl}oxy)benzamide (251)

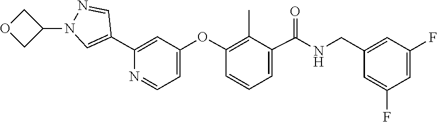 [SMILES: Cc1c(Oc2ccnc(-c3cnn(C4COC4)c3)c2)cccc1C(=O)NCc1cc(F)cc(F)c1]

Compound 251 was synthesized from intermediate 214 (0.05 mmol) and 3-bromooxetane (0.06 mmol) as a white solid in 35% yield according to the general method I. ¹H NMR (400 MHz, DMSO-d₆) δ (ppm): 8.99 (bs, 1H), 8.46 (s, 1H), 8.38 (d, J=5.3 Hz, 1H), 8.13 (s, 1H), 7.41-7.31 (m, 3H), 7.23 (d, J=7.0 Hz, 1H), 7.15-7.06 (m, 3H), 6.51 (d, J=3.6 Hz, 1H), 5.62-5.57 (m, 1H), 4.93-4.91 (m, 4H), 4.48 (d, J=5.4 Hz, 2H), 2.14 (s, 3H).

ESI-MS: 477.10 (M+H)⁺.

### N-[(3,5-difluorophenyl)methyl]-3-({2-[1-(2-methoxyethyl) pyrazol-4-yl]-4-pyridyl} oxy)-2-methyl-benzamide (252)

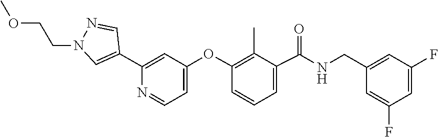 [SMILES: COCCn1cc(-c2cc(Oc3cccc(C(=O)NCc4cc(F)cc(F)c4)c3C)ccn2)cn1]

Compound 252 was synthesized from intermediate 214 (0.05 mmol) and 2-bromoethyl methyl ether (0.06 mmol) as a white solid in 78% yield according to the general method I. ¹H NMR (400 MHz, DMSO-d₆) δ (ppm): 8.99 (t, J=5.8 Hz, 1H), 8.36 (d, J=5.7 Hz, 1H), 8.26 (s, 1H), 7.99 (s, 1H), 7.41-7.35 (m, 2H), 7.27-7.22 (m, 2H), 7.15-7.05 (m, 3H), 6.49 (d, J=5.0 Hz, 1H), 4.48 (d, J=5.9 Hz, 2H), 4.28 (t, J=5.1 Hz, 2H), 3.70 (t, J=5.1 Hz, 2H), 3.23 (s, 3H), 2.14 (s, 3H).

ESI-MS: 479.10 (M+H)⁺.

### N-[(3,5-difluorophenyl)methyl]-3-({2-[1-(dimethylphosphorylmethyl)pyrazol-4-yl]-4-pyridyl}oxy)-2-methyl-benzamide (253)

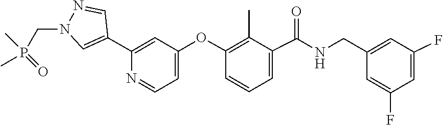 [SMILES: Cc1c(Oc2ccnc(-c3cnn(CP(C)(C)=O)c3)c2)cccc1C(=O)NCc1cc(F)cc(F)c1]

Compound 253 was synthesized from intermediate 214 (0.05 mmol) and 1-{[(dimethylphosphoryl)methoxy]sulfonyl}-4-methylbenzene (0.06 mmol) as a white solid in 75% yield according to the general method I. ¹H NMR (400 MHz, DMSO-d₆) δ (ppm): 9.00 (t, J=6.0 Hz, 1H), 8.38 (d, J=5.7 Hz, 1H), 8.28 (s, 1H), 8.07 (s, 1H), 7.41-7.35 (m, 2H), 7.28 (d, J=2.3 Hz, 1H), 7.23 (dd, J=7.4 Hz, 1.8 Hz, 1H), 7.13 (tt, J=9.5 Hz, 2.3 Hz, 1H), 7.09-7.04 (m, 2H), 6.52 (dd, J=5.7 Hz, 2.4 Hz, 1H), 4.69 (d, J=7.5 Hz, 2H), 4.48 (d, J=6.0 Hz, 2H), 2.14 (s, 3H), 1.46 (s, 3H), 1.42 (s, 3H). ³¹P NMR (162 MHz, DMSO-d₆) δ (ppm): 38.43.

ESI-MS: 511.10 (M+H)⁺.

### 3-({2-[1-(ditert-butoxyphosphorylmethyl)pyrazol-4-yl]-4-pyridyl}oxy)-N-[(3,5-difluorophenyl)methyl]-2-methyl-benzamide (254)

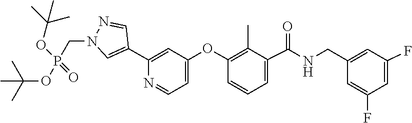 [SMILES: Cc1c(Oc2ccnc(-c3cnn(CP(=O)(OC(C)(C)C)OC(C)(C)C)c3)c2)cccc1C(=O)NCc1cc(F)cc(F)c1]

Compound 254 was synthesized from intermediate 214 (0.12 mmol) and (di-tert-butoxyphosphoryl) methyl 4-methylbenzenesulfonate (0.24 mmol) as a white solid in 51% yield according to the general method I. ¹H NMR (400 MHz, DMSO-d₆) δ (ppm): 8.99 (t, J=6.0 Hz, 1H), 8.37 (d, J=5.7 Hz, 1H), 8.18 (s, 1H), 8.01 (s, 1H), 7.41-7.35 (m, 2H), 7.24 (dd, J=7.2 Hz, 2.2 Hz, 2H), 7.16-7.10 (m, 1H), 7.09-7.04 (m, 2H), 6.52 (dd, J=5.7 Hz, 2.4 Hz, 1H), 4.53 (d, J=11.8 Hz, 2H), 4.48 (d, J=6.0 Hz, 2H), 2.15 (s, 3H), 1.39 (s, 18H). ³¹P NMR (162 MHz, DMSO-d₆) δ (ppm): 10.07.

ESI-MS: 627.15 (M+H)⁺.

### 3-({2-[1-(cyclopropylmethyl) pyrazol-4-yl]-4-pyridyl}oxy)-N-[(3,5-difluorophenyl)methyl]-2-methyl-benzamide (255)

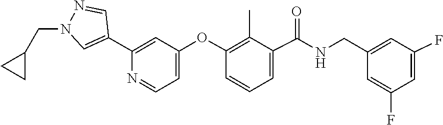 [SMILES: Cc1c(Oc2ccnc(-c3cnn(CC4CC4)c3)c2)cccc1C(=O)NCc1cc(F)cc(F)c1]

Compound 255 was synthesized from intermediate 214 (0.05 mmol) and cyclopropylmethyl bromide (0.06 mmol) as a white solid in 56% yield according to the general method I. ¹H NMR (400 MHz, DMSO-d₆) δ (ppm): 8.99 (t, J=6.0 Hz, 1H), 8.36 (d, J=5.7 Hz, 1H), 8.33 (s, 1H), 7.98-7.97 (m, 1H), 7.41-7.35 (m, 2H), 7.28 (d, J=2.3 Hz, 1H), 7.22 (dd, J=7.4 Hz, 1.9 Hz, 1H), 7.13 (tt, J=9.5 Hz, 2.4 Hz, 1H), 7.09-7.04 (m, 2H), 6.48 (dd, J=5.7 Hz, 2.4 Hz, 1H), 4.48 (d, J=6.0 Hz, 2H), 3.99 (d, J=7.1 Hz, 2H), 2.15 (s, 3H), 1.31-1.21 (m, 1H), 0.56-0.51 (m, 2H), 0.40-0.36 (m, 2H).

ESI-MS: 475.10 (M+H)⁺.

### 3-({2-[1-(2,2-difluoroethyl) pyrazol-4-yl]-4-pyridyl}oxy)-N-[(3,5-difluorophenyl) methyl]-2-methyl-benzamide (256)

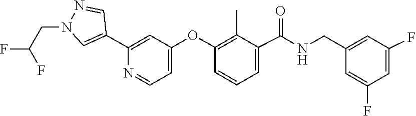 [SMILES: Cc1c(Oc2ccnc(-c3cnn(CC(F)F)c3)c2)cccc1C(=O)NCc1cc(F)cc(F)c1]

Compound 256 was synthesized from intermediate 214 (0.05 mmol) and 2-iodo-1,1-difluoroethane (0.06 mmol) as a white solid in 50% yield according to the general method I. ¹H NMR (400 MHz, DMSO-d₆) δ (ppm): 8.99 (t, J=6.0 Hz, 1H), 8.38 (d, J=5.7 Hz, 1H), 8.35 (s, 1H), 8.09 (d, J=0.4 Hz, 1H), 7.42-7.35 (m, 2H), 7.30 (d, J=2.3 Hz, 1H), 7.23 (dd, J=7.3 Hz, 1.9 Hz, 1H), 7.13 (tt, J=9.5 Hz, 2.4 Hz, 1H), 7.09-7.04 (m, 2H), 6.54-6.25 (m, 2H), 4.66 (td, J=15.1 Hz, 3.7 Hz, 2H), 4.48 (d, J=6.0 Hz, 2H), 2.14 (s, 3H).

ESI-MS: 485.10 (M+H)⁺.

### 3-{[2-(1-cyclobutylpyrazol-4-yl)-4-pyridyl]oxy}-N-[(3,5-difluorophenyl)methyl]-2-methyl-benzamide (257)

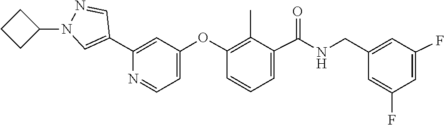 [SMILES: Cc1c(Oc2ccnc(-c3cnn(C4CCC4)c3)c2)cccc1C(=O)NCc1cc(F)cc(F)c1]

Compound 257 was synthesized from intermediate 214 (0.05 mmol) and cyclobutyl bromide (0.06 mmol) as a white solid in 39% yield according to the general method I. ¹H NMR (400 MHz, DMSO-d₆) δ (ppm): 8.99 (t, J=6.0 Hz, 1H), 8.39 (d, J=0.5 Hz, 1H), 8.37-8.35 (m, 1H), 8.01 (d, J=0.5 Hz, 1H), 7.41-7.34 (m, 2H), 7.29 (d, J=2.1 Hz, 1H), 7.22 (dd, J=7.4 Hz, 1.9 Hz, 1H), 7.13 (tt, J=9.4 Hz, 2.4 Hz, 1H), 7.09-7.04 (m, 2H), 6.48 (dd, J=5.7 Hz, 2.4 Hz, 1H), 4.85 (p, J=8.5 Hz, 1H), 4.48 (d, J=6.0 Hz, 2H), 2.50-2.35 (m, 4H), 2.14 (s, 3H), 1.82-1.73 (m, 2H).

ESI-MS: 475.10 (M+H)⁺.

### 3-({2-[1-(cyclobutylmethyl) pyrazol-4-yl]-4-pyridyl}oxy)-N-[(3,5-difluorophenyl)methyl]-2-methyl-benzamide (258)

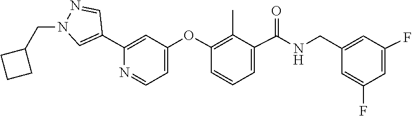 [SMILES: Cc1c(Oc2ccnc(-c3cnn(CC4CCC4)c3)c2)cccc1C(=O)NCc1cc(F)cc(F)c1]

Compound 258 was synthesized from intermediate 214 (0.05 mmol) and (bromomethyl)cyclobutane (0.06 mmol) as a white solid in 43% yield according to the general method I. ¹H NMR (400 MHz, DMSO-d₆) δ (ppm): 8.99 (t, J=6.0 Hz, 1H), 8.36-8.35 (m, 1H), 8.27 (d, J=0.5 Hz, 1H), 7.96 (d, J=0.6 Hz, 1H), 7.41-7.35 (m, 2H), 7.27 (d, J=2.2 Hz, 1H), 7.22 (dd, J=7.4 Hz, 1.9 Hz, 1H), 7.13 (tt, J=9.4 Hz, 2.4 Hz, 1H), 7.09-7.04 (m, 2H), 6.47 (dd, J=5.7 Hz, 2.4 Hz, 1H), 4.48 (d, J=6.0 Hz, 2H), 4.14 (d, J=7.3 Hz, 2H), 2.76 (dt, J=14.9 Hz, 7.5 Hz, 1H), 2.14 (s, 3H), 2.00-1.94 (m, 2H), 1.89-1.72 (m, 4H).

ESI-MS: 489.10 (M+H)⁺.

### 3-({2-[1-(2-cyanoethyl) pyrazol-4-yl]-4-pyridyl}oxy)-N-[(3,5-difluorophenyl)methyl]-2-methyl-benzamide (259)

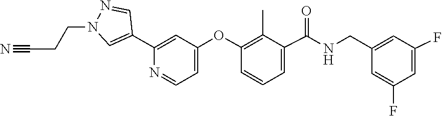 [SMILES: Cc1c(Oc2ccnc(-c3cnn(CCC#N)c3)c2)cccc1C(=O)NCc1cc(F)cc(F)c1]

Compound 259 was synthesized from intermediate 214 (0.05 mmol) and 3-bromopropionitrile (0.06 mmol) as a white solid in 56% yield according to the general method I. ¹H NMR (400 MHz, DMSO-d₆) δ (ppm): 8.99 (t, J=6.1 Hz, 1H), 8.39-8.37 (m, 2H), 8.07 (d, J=0.6 Hz, 1H), 7.42-7.35 (m, 2H), 7.28 (d, J=2.2 Hz, 1H), 7.23 (dd, J=7.4 Hz, 1.9 Hz, 1H), 7.13 (tt, J=9.4 Hz, 2.4 Hz, 1H), 7.09-7.04 (m, 2H), 6.52 (dd, J=5.7 Hz, 2.4 Hz, 1H), 4.48 (d, J=6.0 Hz, 2H), 4.42 (t, J=6.4 Hz, 2H), 3.10 (t, J=6.4 Hz, 2H), 2.15 (s, 3H).

ESI-MS: 474.10 (M+H)⁺.

### 3-({2-[1-(cyanomethyl) pyrazol-4-yl]-4-pyridyl}oxy)-N-[(3,5-difluorophenyl)methyl]-2-methyl-benzamide (260)

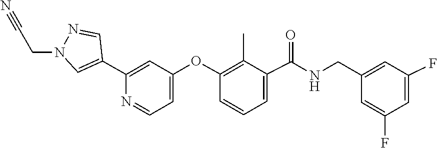 [SMILES: Cc1c(Oc2ccnc(-c3cnn(CC#N)c3)c2)cccc1C(=O)NCc1cc(F)cc(F)c1]

Compound 260 was synthesized from intermediate 214 (0.05 mmol) and 2-iodoacetonitrile (0.07 mmol) as a white solid in 14% yield according to the general method I. ¹H NMR (400 MHz, DMSO-d₆) δ (ppm): 8.99 (t, J=6.0 Hz, 1H), 8.40-8.38 (m, 2H), 8.15 (d, J=0.6 Hz, 1H), 7.42-7.35 (m, 2H), 7.32 (d, J=2.2 Hz, 1H), 7.24 (dd, J=7.3 Hz, 2.0 Hz, 1H), 7.13 (tt, J=9.4 Hz, 2.4 Hz, 1H), 7.09-7.03 (m, 2H), 6.54 (dd, J=5.7 Hz, 2.4 Hz, 1H), 5.53 (s, 2H), 4.48 (d, J=6.0 Hz, 2H), 2.14 (s, 3H).

ESI-MS: 460.10 (M+H)⁺.

### tert-butyl 4-[4-(4-{3-[(3,5-difluorophenyl)methylcarbamoyl]-2-methyl-phenoxy}-2-pyridyl) pyrazol-1-yl]piperidine-1-carboxylate (261)

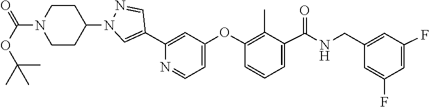 [SMILES: Cc1c(Oc2ccnc(-c3cnn(C4CCN(C(=O)OC(C)(C)C)CC4)c3)c2)cccc1C(=O)NCc1cc(F)cc(F)c1]

Compound 261 was synthesized from intermediate 214 (0.10 mmol) and tert-butyl 4-[(methylsulfonyl)oxy]piperidine-1-carboxylate (0.11 mmol) as a white solid in 70% yield according to the general method I. ¹H NMR (400 MHz, DMSO-d₆) δ (ppm): 8.99 (t, J=6.0 Hz, 1H), 8.37-8.35 (m, 2H), 8.00 (s, 1H), 7.41-7.35 (m, 2H), 7.29 (d, J=2.3 Hz, 1H), 7.22 (dd, J=7.4 Hz, 1.9 Hz, 1H), 7.13 (tt, J=9.5 Hz, 2.4 Hz, 1H), 7.10-7.04 (m, 2H), 6.48 (dd, J=5.7 Hz, 2.4 Hz, 1H), 4.48 (d, J=6.0 Hz, 2H), 4.42-4.34 (m, 1H), 4.04 (d, J=11.8 Hz, 2H), 2.91 (bs, 2H), 2.14 (s, 3H), 2.02 (d, J=10.2 Hz, 2H), 1.85-1.75 (m, 2H), 1.42 (s, 9H).

ESI-MS: 604.40 (M+H)⁺.

### N-[(3,5-difluorophenyl)methyl]-2-methyl-3-({2-[1-(4-piperidyl) pyrazol-4-yl]-4-pyridyl}oxy)benzamide (262)

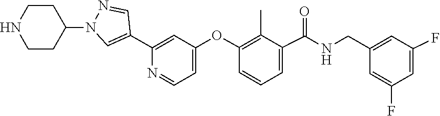 [SMILES: Cc1c(Oc2ccnc(-c3cnn(C4CCNCC4)c3)c2)cccc1C(=O)NCc1cc(F)cc(F)c1]

To a stirred solution of compound 261 (25 mg, 0.04 mmol) in dioxane (2 mL) was added HCl (4N in dioxane, 0.41 mL, 10 equiv.) and the mixture was stirred at room temperature overnight. The reaction mixture was diluted with EtOAc and washed with a saturated solution of NaHCO₃. The organic layer was dried over Na₂SO₄, filtered and concentrated under reduced pressure. The crude was purified by flash column chromatography (DCM/MeOH from 100/0 to 90/10) and reverse phase chromatography (H₂O/MeOH from 100/0 to 0/100) to give the expected compound as a white solid in 33% yield. ¹H NMR (400 MHz, DMSO-d₆) δ (ppm): 8.99 (t, J=6.0 Hz, 1H), 8.36 (d, J=5.7 Hz, 1H), 8.32 (s, 1H), 7.99 (s, 1H), 7.41-7.34 (m, 2H), 7.29 (d, J=2.3 Hz, 1H), 7.22 (dd, J=7.3 Hz, 1.8 Hz, 1H), 7.16-7.10 (m, 1H), 7.09-7.14 (m, 2H), 6.48 (dd, J=5.7 Hz, 2.4 Hz, 1H), 4.48 (d, J=6.0 Hz, 2H), 4.28-4.19 (m, 1H), 3.07 (d, J=12.3 Hz, 2H), 2.61 (d, J=10.8, 2H), 2.14 (s, 3H), 1.98 (d, J=10.2, 2H), 1.87-1.78 (m, 2H).

ESI-MS: 504.10 (M+H)⁺.

### Example 9: General Procedure for the Synthesis of Analogues 263-277

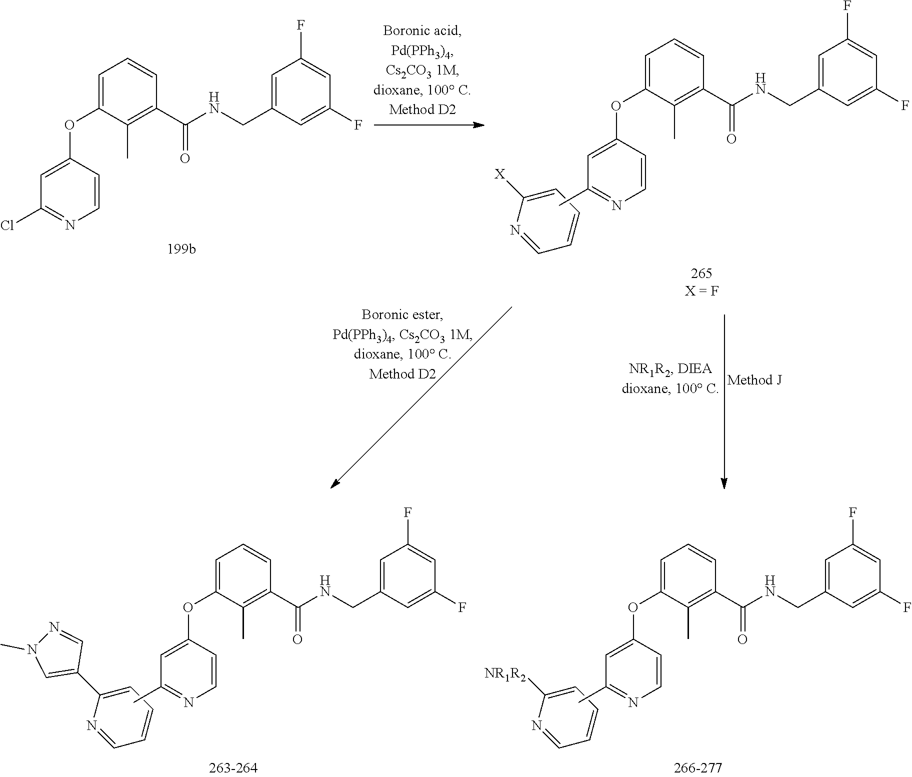

### N-[(3,5-difluorophenyl)methyl]-2-methyl-3-({2-[2-(1-methylpyrazol-4-yl)-4-pyridyl]-4-pyridyl}oxy)benzamide (263)

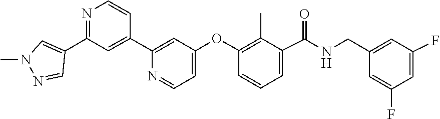 [SMILES: Cc1c(Oc2ccnc(-c3ccnc(-c4cnn(C)c4)c3)c2)cccc1C(=O)NCc1cc(F)cc(F)c1]

Compound 263 was synthesized in a two steps procedure from intermediate 199b (0.08 mmol), 2-chloropyridine-4-boronic acid (0.08 mmol) and 1-methylpyrazole-4-boronic acid pinacol ester (0.15 mmol) as a white solid in 23% yield according to the general method D2. ¹H NMR (400 MHz, DMSO-d₆) δ (ppm): 9.01 (t, J=6.1 Hz, 1H), 8.61-8.59 (m, 2H), 8.40 (s, 1H), 8.29-8.28 (m, 1H), 8.10 (d, J=0.7 Hz, 1H), 7.92 (d, J=2.3 Hz, 1H), 7.81 (dd, J=5.2 Hz, 1.7 Hz, 1H), 7.44-7.38 (m, 2H), 7.29 (dd, J=7.3 Hz, 2.0 Hz, 1H), 7.16-7.10 (m, 1H), 7.09-7.04 (m, 2H), 6.71 (dd, J=5.6 Hz, 2.4 Hz, 1H), 4.48 (d, J=6.0 Hz, 2H), 3.90 (s, 3H), 2.17 (s, 3H).

ESI-MS: 512.10 (M+H)⁺.

### N-[(3,5-difluorophenyl)methyl]-2-methyl-3-({2-[6-(1-methylpyrazol-4-yl)-3-pyridyl]-4-pyridyl}oxy)benzamide (264)

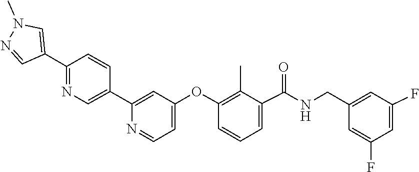 [SMILES: Cc1c(Oc2ccnc(-c3ccc(-c4cnn(C)c4)nc3)c2)cccc1C(=O)NCc1cc(F)cc(F)c1]

Compound 264 was synthesized in a two steps procedure from intermediate 199b (0.08 mmol), (6-chloropyridin-3-yl) boronic acid (0.08 mmol) and 1-methylpyrazole-4-boronic acid pinacol ester (0.15 mmol) as a white solid in 15% yield according to the general method D2. ¹H NMR (400 MHz, DMSO-d₆) δ (ppm): 9.16 (dd, J=2.3 Hz, 0.7 Hz, 1H), 9.01 (t, J=6.1 Hz, 1H), 8.55 (d, J=5.7 Hz, 1H), 8.38 (dd, J=8.4 Hz, 2.4 Hz, 1H), 8.35 (s, 1H), 8.06 (d, J=0.6 Hz, 1H), 7.74 (dd, J=8.3 Hz, 0.6 Hz, 1H), 7.65 (d, J=2.2 Hz, 1H), 7.44-7.37 (m, 2H), 7.27 (dd, J=7.3 Hz, 2.0 Hz, 1H), 7.16-7.10 (m, 1H), 7.09-7.04 (m, 2H), 6.67 (dd, J=5.7 Hz, 2.4 Hz, 1H), 4.48 (d, J=6.0 Hz, 2H), 3.90 (s, 3H), 2.16 (s, 3H).

ESI-MS: 512.10 (M+H)⁺.

### N-[(3,5-difluorophenyl)methyl]-3-{[2-(2-fluoro-4-pyridyl)-4-pyridyl]oxy}-2-methyl-benzamide (265a)

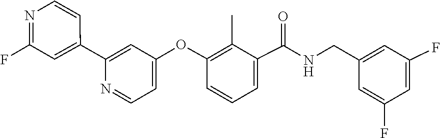 [SMILES: Cc1c(Oc2ccnc(-c3ccnc(F)c3)c2)cccc1C(=O)NCc1cc(F)cc(F)c1]

Compound 265a was synthesized from intermediate 199b (1.16 mmol) and 2-fluoropyridin-4-ylboronic acid (1.39 mmol) as a white solid in 100% yield according to the general method D2. ¹H NMR (400 MHz, DMSO-d₆) δ (ppm): 9.00 (t, J=5.9 Hz, 1H), 8.61 (d, J=5.6 Hz, 1H), 8.36 (d, J=5.3 Hz, 1H), 8.02 (d, J=5.2 Hz, 1H), 7.85 (d, J=2. Hz 3, 1H), 7.82 (s, 1H), 7.44-7.38 (m, 2H), 7.28 (dd, J=6.9 Hz, 2.2 Hz, 1H), 7.16-7.04 (m, 3H), 6.78 (dd, J=5.6 Hz, 1.9 Hz, 1H), 4.48 (d, J=5.9 Hz, 2H), 2.16 (s, 3H).

ESI-MS: 450.05 (M+H)⁺.

### N-[(3,5-difluorophenyl)methyl]-3-{[2-(6-fluoro-3-pyridyl)-4-pyridyl]oxy}-2-methyl-benzamide (265b)

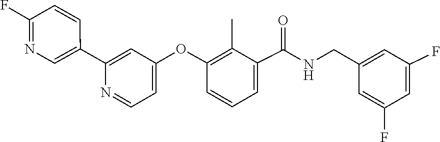 [SMILES: Cc1c(Oc2ccnc(-c3ccc(F)nc3)c2)cccc1C(=O)NCc1cc(F)cc(F)c1]

Compound 265b was synthesized from intermediate 199b (0.77 mmol) and 6-fluoro-3-pyridinylboronic acid (0.93 mmol) as a white solid in 100% yield according to the general method D2. ¹H NMR (400 MHz, DMSO-d₆) δ (ppm): 9.00 (t, J=6.0 Hz, 1H), 8.90 (d, J=2.5 Hz, 1H), 8.64-8.59 (m, 1H), 8.57-8.55 (m, 1H), 7.69 (d, J=2.1 Hz, 1H), 7.43-7.37 (m, 2H), 7.31 (dd, J=8.6 Hz, 2.4 Hz, 1H), 7.27 (dd, J=7.2 Hz, 2.1 Hz, 1H), 7.13 (tt, J=9.4 Hz, 2.4 Hz, 1H), 7.09-7.04 (m, 2H), 6.70 (dd, J=5.7 Hz, 2.4 Hz, 1H), 4.48 (d, J=6.0 Hz, 2H), 2.16 (s, 3H).

ESI-MS: 450.05 (M+H)⁺.
Method J: To a solution of 265 (1 equiv.) in dioxane (10 mL/mmol) were added amine derivative (16 equiv.) and DIEA (6 equiv.). The mixture was stirred at 100° C. until completion (from 2 h to overnight). The reaction mixture was concentrated under reduced pressure. The crude was purified by flash column chromatography (DCM/MeOH from 100/0 to 90/10) and reverse phase chromatography (H₂O/MeOH from 100/0 to 0/100) to give the expected compound.

The following compound 266 is an example illustrating Method J:

### N-[(3,5-difluorophenyl)methyl]-2-methyl-3-{[2-(2-pyrrolidin-1-yl-4-pyridyl)-4-pyridyl]oxybenzamide (266)

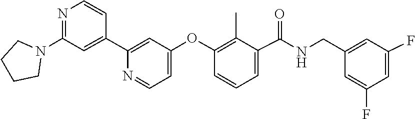 [SMILES: Cc1c(Oc2ccnc(-c3ccnc(N4CCCC4)c3)c2)cccc1C(=O)NCc1cc(F)cc(F)c1]

Compound 266 was synthesized from intermediate 265a (0.07 mmol) and pyrrolidine (0.53 mmol) as a white solid in 55% yield according to the general method J. ¹H NMR (400 MHz, DMSO-d₆) δ (ppm): 9.01 (t, J=6.0 Hz, 1H), 8.55 (d, J=5.8 Hz, 1H), 8.14 (dd, J=5.3 Hz, 0.6 Hz, 1H), 7.64 (d, J=2.2 Hz, 1H), 7.43-7.37 (m, 2H), 7.27 (dd, J=7.3 Hz, 2.0 Hz, 1H), 7.16-7.04 (m, 5H), 6.71 (dd, J=5.6 Hz, 2.4 Hz, 1H), 4.48 (d, J=6.0 Hz, 2H), 3.46-3.43 (m, 4H), 2.15 (s, 3H), 1.98-1.94 (m, 4H).

ESI-MS: 501.10 (M+H)⁺.

### N-[(3,5-difluorophenyl)methyl]-2-methyl-3-({2-[2-(1-piperidyl)-4-pyridyl]-4-pyridyl}oxy)benzamide (267)

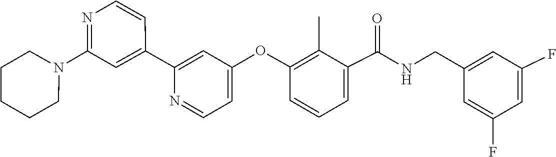 [SMILES: Cc1c(Oc2ccnc(-c3ccnc(N4CCCCC4)c3)c2)cccc1C(=O)NCc1cc(F)cc(F)c1]

Compound 267 was synthesized from intermediate 265a (0.07 mmol) and piperidine (0.53 mmol) as a white solid in 50% yield according to the general method J. ¹H NMR (400 MHz, DMSO-d₆) δ (ppm): 9.01 (t, J=6.0 Hz, 1H), 8.55 (d, J=5.6 Hz, 1H), 8.18 (d, J=5.2 Hz, 1H), 7.70 (d, J=2.3 Hz, 1H), 7.43-7.36 (m, 3H), 7.26 (dd, J=7.2 Hz, 2.0 Hz, 1H), 7.17-7.04 (m, 4H), 6.69 (dd, J=5.6 Hz, 2.4 Hz, 1H), 4.48 (d, J=6.0 Hz, 2H), 3.60-3.58 (m, 4H), 2.16 (s, 3H), 1.62-1.55 (m, 6H).

ESI-MS: 515.15 (M+H)⁺.

### N-[(3,5-difluorophenyl)methyl]-3-({2-[2-(dimethylamino)-4-pyridyl]-4-pyridyl}oxy)-2-methyl-benzamide (268)

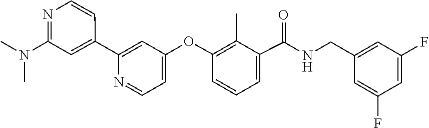 [SMILES: Cc1c(Oc2ccnc(-c3ccnc(N(C)C)c3)c2)cccc1C(=O)NCc1cc(F)cc(F)c1]

Compound 268 was synthesized from intermediate 265a (0.07 mmol) and dimethylamine (2M THF, 0.67 mmol) as a white solid in 47% yield according to the general method J. ¹H NMR (400 MHz, DMSO-d₆) δ (ppm): 9.01 (t, J=6.0 Hz, 1H), 8.56 (d, J=5.6 Hz, 1H), 8.18-8.16 (m, 1H), 7.66 (d, J=2.3 Hz, 1H), 7.43-7.37 (m, 2H), 7.27 (dd, J=8.1 Hz, 2.7 Hz, 2H), 7.15-7.04 (m, 4H), 6.71 (dd, J=5.6 Hz, 2.4 Hz, 1H), 4.48 (d, J=6.0 Hz, 2H), 3.09 (s, 6H), 2.15 (s, 3H).

ESI-MS: 475.10 (M+H)⁺.

### N-[(3,5-difluorophenyl)methyl]-2-methyl-3-{[2-(2-morpholino-4-pyridyl)-4-pyridyl]oxy}benzamide (269)

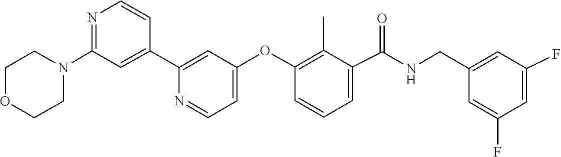 [SMILES: Cc1c(Oc2ccnc(-c3ccnc(N4CCOCC4)c3)c2)cccc1C(=O)NCc1cc(F)cc(F)c1]

Compound 269 was synthesized from intermediate 265a (0.07 mmol) and morpholine (1.07 mmol) as a white solid in 50% yield according to the general method J. ¹H NMR (400 MHz, DMSO-d₆) δ (ppm): 9.01 (t, J=6.1 Hz, 1H), 8.55 (d, J=5.7 Hz, 1H), 8.23 (d, J=5.6 Hz, 1H), 7.75 (d, J=2.2 Hz, 1H), 7.46 (s, 1H), 7.43-7.37 (m, 2H), 7.29 (dd, J=5.2 Hz, 1.3 Hz, 1H), 7.26 (dd, J=7.3 Hz, 2.0 Hz, 1H), 7.13 (tt, J=9.4 Hz, 2.4 Hz, 1H), 7.09-7.04 (m, 2H), 6.69 (dd, J=5.6 Hz, 2.4 Hz, 1H), 4.48 (d, J=6.0 Hz, 2H), 3.73-3.71 (m, 4H), 3.54-3.51 (m, 4H), 2.16 (s, 3H).

ESI-MS: 517.15 (M+H)⁺.

### N-[(3,5-difluorophenyl)methyl]-2-methyl-3-({2-[2-(4-methylpiperazin-1-yl)-4-pyridyl]-4-pyridyl}oxy)benzamide (270)

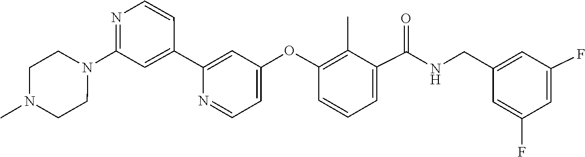 [SMILES: Cc1c(Oc2ccnc(-c3ccnc(N4CCN(C)CC4)c3)c2)cccc1C(=O)NCc1cc(F)cc(F)c1]

Compound 270 was synthesized from intermediate 265a (0.07 mmol) and 1-methylpiperazine (1.07 mmol) as a white solid in 40% yield according to the general method J. ¹H NMR (400 MHz, DMSO-d₆) δ (ppm): 9.01 (t, J=6.0 Hz, 1H), 8.55 (d, J=5.8 Hz, 1H), 8.21-8.19 (m, 1H), 7.74 (d, J=2.2 Hz, 1H), 7.45 (s, 1H), 7.43-7.37 (m, 2H), 7.27-7.23 (m, 2H), 7.13 (tt, J=9.4 Hz, 2.4 Hz, 1H), 7.09-7.04 (m, 2H), 6.69 (dd, J=5.6 Hz, 2.4 Hz, 1H), 4.48 (d, J=6.0 Hz, 2H), 3.57-3.55 (m, 4H), 2.42-2.40 (m, 4H), 2.22 (s, 3H), 2.16 (s, 3H).

ESI-MS: 530.15 (M+H)⁺.

### 3-{[2-(2-amino-4-pyridyl)-4-pyridyl]oxy}-N-[(3,5-difluorophenyl)methyl]-2-methyl-benzamide (271)

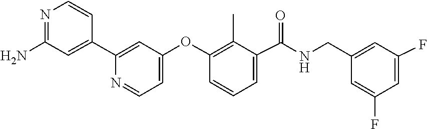 [SMILES: Cc1c(Oc2ccnc(-c3ccnc(N)c3)c2)cccc1C(=O)NCc1cc(F)cc(F)c1]

Compound 271 was synthesized from intermediate 265a (0.07 mmol) and NH₄OH (1.5 mL) as a white solid in 34% yield according to the general method J. ¹H NMR (400 MHz, DMSO-d₆) δ (ppm): 9.01 (t, J=6.1 Hz, 1H), 8.55 (d, J=5.8 Hz, 1H), 7.98 (dd, J=5.4 Hz, 0.6 Hz, 1H), 7.43-7.37 (m, 3H), 7.28 (dd, J=7.3 Hz, 2.0 Hz, 1H), 7.16-7.04 (m, 4H), 7.02 (dd, J=5.4 Hz, 1.6 Hz, 1H), 6.78 (dd, J=5.6 Hz, 2.4 Hz, 1H), 6.03 (s, 2H), 4.48 (d, J=6.0 Hz, 2H), 2.15 (s, 3H).

ESI-MS: 447.05 (M+H)⁺.

### N-[(3,5-difluorophenyl)methyl]-2-methyl-3-{[2-(6-pyrrolidin-1-yl-3-pyridyl)-4-pyridyl]oxy}benzamide (272)

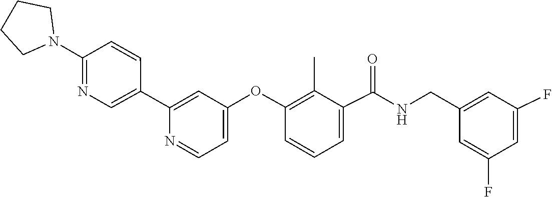 [SMILES: Cc1c(Oc2ccnc(-c3ccc(N4CCCC4)nc3)c2)cccc1C(=O)NCc1cc(F)cc(F)c1]

Compound 272 was synthesized from intermediate 265b (0.07 mmol) and pyrrolidine (1.06 mmol) as a white solid in 55% yield according to the general method J. ¹H NMR (400 MHz, DMSO-d₆) δ (ppm): 9.01 (t, J=6.0 Hz, 1H), 8.75 (d, J=1.9 Hz, 1H), 8.43 (d, J=5.6 Hz, 1H), 8.13 (dd, J=8.9 Hz, 2.5 Hz, 1H), 7.42-7.35 (m, 3H), 7.24 (dd, J=7.5 Hz, 1.8 Hz, 1H), 7.16-7.10 (m, 1H), 7.09-7.04 (m, 2H), 6.55-6.50 (m, 2H), 4.48 (d, J=6.0 Hz, 2H), 3.45-3.42 (m, 4H), 2.15 (s, 3H), 1.97-1.94 (m, 4H).

ESI-MS: 501.10 (M+H)⁺.

### N-[(3,5-difluorophenyl)methyl]-2-methyl-3-({2-[6-(1-piperidyl)-3-pyridyl]-4-pyridyl}oxy)benzamide (273)

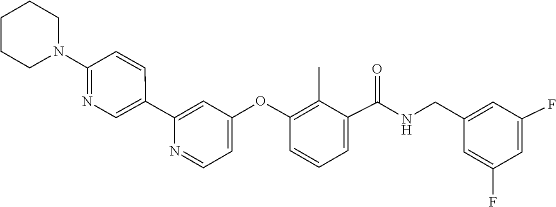 [SMILES: Cc1c(Oc2ccnc(-c3ccc(N4CCCCC4)nc3)c2)cccc1C(=O)NCc1cc(F)cc(F)c1]

Compound 273 was synthesized from intermediate 265b (0.07 mmol) and piperidine (1.06 mmol) as a white solid in 65% yield according to the general method J. ¹H NMR (400 MHz, DMSO-d₆) δ (ppm): 9.00 (t, J=6.0 Hz, 1H), 8.75 (d, J=2.2 Hz, 1H), 8.44 (d, J=5.8 Hz, 1H), 8.13 (dd, J=9.0 Hz, 2.5 Hz, 1H), 7.42-7.35 (m, 3H), 7.24 (dd, J=7.4 Hz, 1.8 Hz, 1H), 7.16-7.04 (m, 3H), 6.87 (d, J=8.9 Hz, 1H), 6.56 (dd, J=5.7 Hz, 2.4 Hz, 1H), 4.48 (d, J=6.0 Hz, 2H), 3.61-3.58 (m, 4H), 2.15 (s, 3H), 1.63-1.54 (m, 6H).

ESI-MS: 515.20 (M+H)⁺.

### N-[(3,5-difluorophenyl)methyl]-2-methyl-3-{[2-(6-morpholino-3-pyridyl)-4-pyridyl]oxy}benzamide (274)

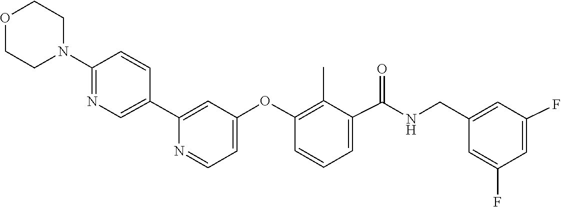 [SMILES: Cc1c(Oc2ccnc(-c3ccc(N4CCOCC4)nc3)c2)cccc1C(=O)NCc1cc(F)cc(F)c1]

Compound 274 was synthesized from intermediate 265b (0.07 mmol) and morpholine (1.06 mmol) as a white solid in 65% yield according to the general method J. ¹H NMR (400 MHz, DMSO-d₆) δ (ppm): 9.00 (t, J=6.0 Hz, 1H), 8.80 (d, J=2.2 Hz, 1H), 8.46 (d, J=5.7 Hz, 1H), 8.20 (dd, J=9.0 Hz, 2.5 Hz, 1H), 7.45 (d, J=2.3 Hz, 1H), 7.40-7.36 (m, 2H), 7.25 (dd, J=7.4 Hz, 1.9 Hz, 1H), 7.13 (tt, J=9.5 Hz, 2.4 Hz, 1H), 7.09-7.04 (m, 2H), 6.91 (d, J=9.0 Hz, 1H), 6.57 (dd, J=5.7 Hz, 2.4 Hz, 1H), 4.48 (d, J=6.0 Hz, 2H), 3.72-3.69 (m, 4H), 3.55-3.52 (m, 4H), 2.15 (s, 3H).

ESI-MS: 517.15 (M+H)⁺.

### N-[(3,5-difluorophenyl)methyl]-2-methyl-3-({2-[6-(4-methylpiperazin-1-yl)-3-pyridyl]-4-pyridyl}oxy)benzamide (275)

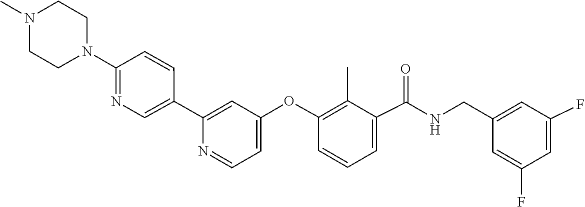 [SMILES: Cc1c(Oc2ccnc(-c3ccc(N4CCN(C)CC4)nc3)c2)cccc1C(=O)NCc1cc(F)cc(F)c1]

Compound 275 was synthesized from intermediate 265b (0.07 mmol) and 1-methylpiperazine (1.06 mmol) as a white solid in 52% yield according to the general method J. ¹H NMR (400 MHz, DMSO-d₆) δ (ppm): 9.00 (t, J=6.0 Hz, 1H), 8.77 (d, J=2.1 Hz, 1H), 8.45 (d, J=5.8 Hz, 1H), 8.16 (dd, J=9.0 Hz, 2.5 Hz, 1H), 7.43 (d, J=2.2 Hz, 1H), 7.42-7.36 (m, 2H), 7.25 (dd, J=7.4 Hz, 1.9 Hz, 1H), 7.16-7.10 (m, 1H), 7.09-7.04 (m, 2H), 6.90 (d, J=9.0 Hz, 1H), 6.57 (dd, J=5.7 Hz, 2.4 Hz, 1H), 4.48 (d, J=6.0 Hz, 2H), 3.58-3.55 (m, 4H), 2.40-2.38 (m, 4H), 2.22 (s, 3H), 2.15 (s, 3H).

ESI-MS: 530.15 (M+H)⁺.

### N-[(3,5-difluorophenyl)methyl]-3-({2-[6-(dimethylamino)-3-pyridyl]-4-pyridyl} oxy)-2-methyl-benzamide (276)

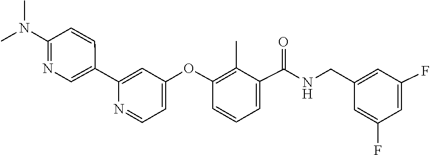 [SMILES: Cc1c(Oc2ccnc(-c3ccc(N(C)C)nc3)c2)cccc1C(=O)NCc1cc(F)cc(F)c1]

Compound 276 was synthesized from intermediate 265b (0.07 mmol) and dimethylamine (2M THF, 1.06 mmol) as a white solid in 72% yield according to the general method J. ¹H NMR (400 MHz, DMSO-d₆) δ (ppm): 9.01 (t, J=6.0 Hz, 1H), 8.76 (dd, J=2.5 Hz, 0.6 Hz, 1H), 8.44 (d, J=5.8 Hz, 1H), 8.14 (dd, J=9.0 Hz, 2.5 Hz, 1H), 7.42-7.35 (m, 3H), 7.24 (dd, J=7.5 Hz, 1.8 Hz, 1H), 7.16-7.10 (m, 1H), 7.09-7.04 (m, 2H), 6.71 (dd, J=9.0 Hz, 0.5 Hz, 1H), 6.55 (dd, J=5.7 Hz, 2.4 Hz, 1H), 4.48 (d, J=6.0 Hz, 2H), 3.08 (s, 6H), 2.15 (s, 3H).

ESI-MS: 475.10 (M+H)⁺.

### 3-{[2-(6-amino-3-pyridyl)-4-pyridyl]oxy}-N-[(3,5-difluorophenyl)methyl]-2-methyl-benzamide (277)

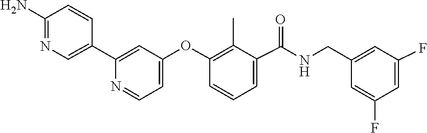 [SMILES: Cc1c(Oc2ccnc(-c3ccc(N)nc3)c2)cccc1C(=O)NCc1cc(F)cc(F)c1]

Compound 277 was synthesized from intermediate 265b (0.07 mmol) and NH₄OH (1.5 mL) as a white solid in 69% yield according to the general method J. ¹H NMR (400 MHz, DMSO-d₆) δ (ppm): 9.00 (t, J=6.0 Hz, 1H), 8.60-8.59 (m, 1H), 8.42 (d, J=5.8 Hz, 1H), 8.01 (dd, J=8.7 Hz, 2.5 Hz, 1H), 7.42-7.35 (m, 2H), 7.34 (d, J=2.2 Hz, 1H), 7.24 (dd, J=7.5 Hz, 1.8 Hz, 1H), 7.13 (tt, J=9.4 Hz, 2.4 Hz, 1H), 7.09-7.04 (m, 2H), 6.55 (dd, J=5.7 Hz, 2.4 Hz, 1H), 6.49 (dd, J=8.7 Hz, 0.6 Hz, 1H), 6.28 (s, 2H), 4.48 (d, J=6.0 Hz, 2H), 2.15 (s, 3H).

ESI-MS: 447.05 (M+H)⁺.

### Example 10: General Procedure for the Synthesis of Analogues 278-283

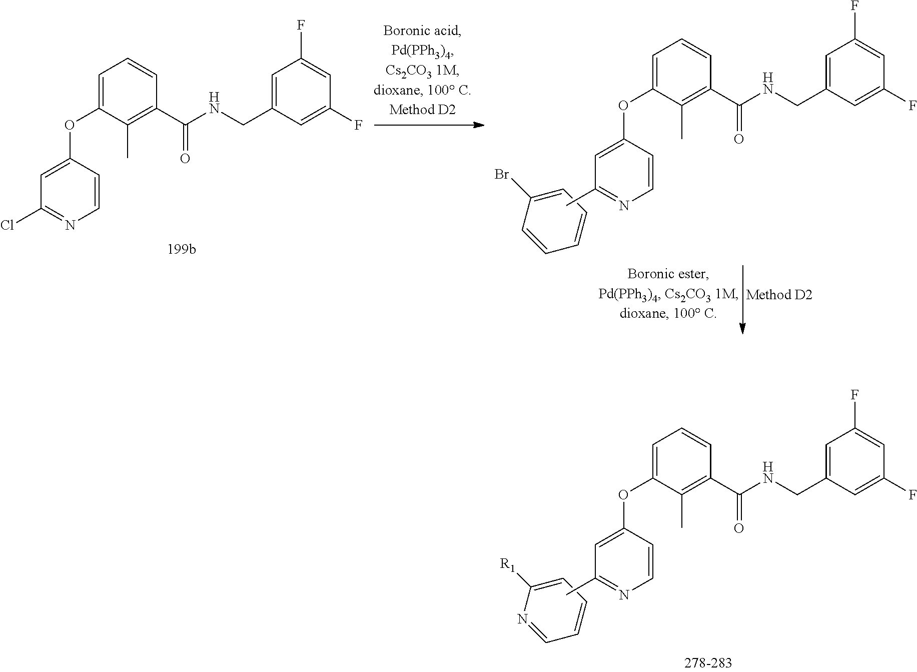

### N-[(3,5-difluorophenyl)methyl]-2-methyl-3-({2-[3-(1-methylpyrazol-4-yl)phenyl]-4-pyridyl}oxy)benzamide (278)

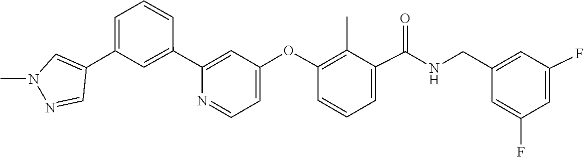 [SMILES: Cc1c(Oc2ccnc(-c3cccc(-c4cnn(C)c4)c3)c2)cccc1C(=O)NCc1cc(F)cc(F)c1]

Compound 278 was synthesized in a two steps procedure from intermediate 199b (0.06 mmol), (3-bromophenyl) boronic acid (0.06 mmol) and 1-methylpyrazole-4-boronic acid pinacol ester (0.13 mmol) as a white solid in 10% yield according to the general method D2. ¹H NMR (600 MHz, DMSO-d₆) δ (ppm): 9.00 (t, J=6.0 Hz, 1H), 8.54 (d, J=5.6 Hz, 1H), 8.26 (s, 1H), 8.23 (t, J=1.6 Hz, 1H), 7.95 (d, J=0.8 Hz, 1H), 7.85-7.83 (m, 1H), 7.66 (d, J=2.3 Hz, 1H), 7.64-7.62 (m, 1H), 7.45 (t, J=7.7 Hz, 1H), 7.43-7.37 (m, 2H), 7.27 (dd, J=7.8 Hz, 1.4 Hz, 1H), 7.12 (tt, J=9.4 Hz, 2.4 Hz, 1H), 7.09-7.05 (m, 2H), 6.66 (dd, J=5.6 Hz, 2.4 Hz, 1H), 4.48 (d, J=6.0 Hz, 2H), 3.88 (s, 3H), 2.17 (s, 3H).

ESI-MS: 511.10 (M+H)⁺.

### N-[(3,5-difluorophenyl)methyl]-2-methyl-3-({2-[3-(2-methylpyrazol-3-yl)phenyl]-4-pyridyl}oxy)benzamide (279)

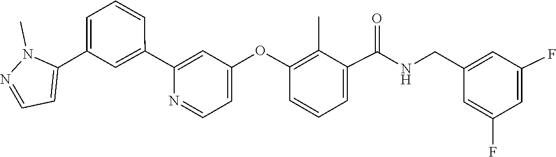 [SMILES: Cc1c(Oc2ccnc(-c3cccc(-c4ccnn4C)c3)c2)cccc1C(=O)NCc1cc(F)cc(F)c1]

Compound 279 was synthesized in a two steps procedure from intermediate 199b (0.08 mmol), (3-bromophenyl) boronic acid (0.08 mmol) and 1-methyl-1H-pyrazole-5-boronic acid pinacol ester (0.15 mmol) as a white solid in 6% yield according to the general method D2. ¹H NMR (600 MHz, DMSO-d₆) δ (ppm): 8.99 (t, J=6.0 Hz, 1H), 8.56-8.55 (m, 1H), 8.15 (t, J=2.0 Hz, 1H), 8.10-8.08 (m, 1H), 7.64 (d, J=2.1 Hz, 1H), 7.62-7.60 (m, 2H), 7.49 (d, J=1.9 Hz, 1H), 7.42-7.37 (m, 2H), 7.27 (dd, J=7.7 Hz, 1.4 Hz, 1H), 7.12 (tt, J=9.4 Hz, 2.4 Hz, 1H), 7.09-7.05 (m, 2H), 6.70 (dd, J=5.6 Hz, 2.4 Hz, 1H), 6.49 (d, J=1.9 Hz, 1H), 4.48 (d, J=6.0 Hz, 2H), 3.88 (s, 3H), 2.16 (s, 3H).

ESI-MS: 511.15 (M+H)⁺.

### N-[(3,5-difluorophenyl)methyl]-2-methyl-3-({2-[3-(1-methylpyrazol-3-yl)phenyl]-4-pyridyl}oxy)benzamide (280)

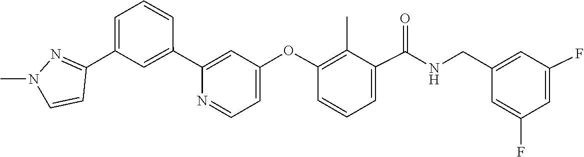 [SMILES: Cc1c(Oc2ccnc(-c3cccc(-c4ccn(C)n4)c3)c2)cccc1C(=O)NCc1cc(F)cc(F)c1]

Compound 280 was synthesized in a two steps procedure from intermediate 199b (0.08 mmol), (3-bromophenyl) boronic acid (0.08 mmol) and 1-methyl-3-(4,4,5,5-tetramethyl-1,3,2-dioxaborolan-2-yl)-1H-pyrazole (0.15 mmol) as a white solid in 10% yield according to the general method D2. ¹H NMR (600 MHz, DMSO-d₆) δ (ppm): 9.00 (t, J=6.0 Hz, 1H), 8.56-8.55 (m, 1H), 8.48 (t, J=1.6 Hz, 1H), 7.90 (ddd, J=7.8 Hz, 1.8 Hz, 1.1 Hz, 1H), 7.85 (ddd, J=7.7 Hz, 1.6 Hz, 1.1 Hz, 1H), 7.75 (d, J=2.2 Hz, 1H), 7.58 (d, J=2.2 Hz, 1H), 7.49 (t, J=7.7 Hz, 1H), 7.43-7.37 (m, 2H), 7.28 (dd, J=7.8 Hz, 1.3 Hz, 1H), 7.12 (tt, J=9.3 Hz, 2.4 Hz, 1H), 7.09-7.05 (m, 2H), 6.78 (d, J=2.2 Hz, 1H), 6.70 (dd, J=5.6 Hz, 2.4 Hz, 1H), 4.48 (d, J=6.0 Hz, 2H), 3.90 (s, 3H), 2.17 (s, 3H).

ESI-MS: 511.10 (M+H)⁺.

### N-[(3,5-difluorophenyl)methyl]-2-methyl-3-({2-[4-(1-methylpyrazol-4-yl)phenyl]-4-pyridyl}oxy)benzamide (281)

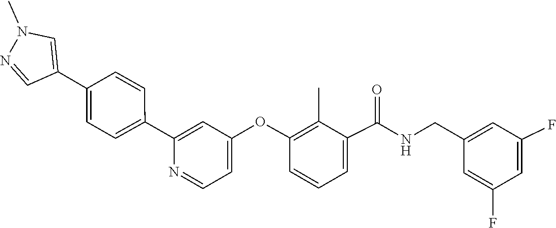 [SMILES: Cc1c(Oc2ccnc(-c3ccc(-c4cnn(C)c4)cc3)c2)cccc1C(=O)NCc1cc(F)cc(F)c1]

Compound 281 was synthesized in a two steps procedure from intermediate 199b (0.10 mmol), (4-bromophenyl) boronic acid (0.15 mmol) and 1-methylpyrazole-4-boronic acid pinacol ester (0.21 mmol) as a white solid in 4% yield according to the general method D2. ¹H NMR (400 MHz, DMSO-d₆) δ (ppm): 9.01 (t, J=6.0 Hz, 1H), 8.52 (d, J=5.6 Hz, 1H), 8.21 (s, 1H), 8.03 (d, J=8.5 Hz, 2H), 7.93 (d, J=0.6 Hz, 1H), 7.66 (d, J=8.5 Hz, 2H), 7.51 (d, J=2.3 Hz, 1H), 7.43-7.37 (m, 2H), 7.27 (dd, J=7.4 Hz, 1.8 Hz, 1H), 7.16-7.10 (m, 1H), 7.09-7.04 (m, 2H), 6.66 (dd, J=5.6 Hz, 2.4 Hz, 1H), 4.48 (d, J=6.0 Hz, 2H), 3.88 (s, 3H), 2.16 (s, 3H).

ESI-MS: 511.10 (M+H)⁺.

### N-[(3,5-difluorophenyl)methyl]-2-methyl-3-({2-[4-(2-methylpyrazol-3-yl)phenyl]-4-pyridyl}oxy)benzamide (282)

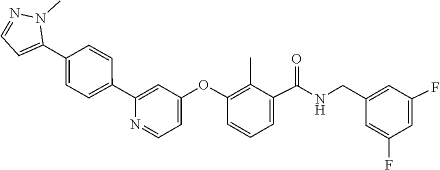 [SMILES: Cc1c(Oc2ccnc(-c3ccc(-c4ccnn4C)cc3)c2)cccc1C(=O)NCc1cc(F)cc(F)c1]

Compound 282 was synthesized in a two steps procedure from intermediate 199b (0.10 mmol), (4-bromophenyl) boronic acid (0.15 mmol) and 1-methyl-1H-pyrazole-5-boronic acid pinacol ester (0.21 mmol) as a white solid in 4% yield according to the general method D2. ¹H NMR (400 MHz, DMSO-d₆) δ (ppm): 9.02 (t, J=6.0 Hz, 1H), 8.57 (d, J=5.7 Hz, 1H), 8.15 (d, J=8.4 Hz, 2H), 7.65 (d, J=8.4 Hz, 2H), 7.56 (d, J=2.3 Hz, 1H), 7.49 (d, J=1.9 Hz, 1H), 7.44-7.37 (m, 2H), 7.29 (dd, J=7.3 Hz, 1.9 Hz, 1H), 7.15-7.10 (m, 1H), 7.09-7.04 (m, 2H), 6.74 (dd, J=5.6 Hz, 2.4 Hz, 1H), 6.48 (d, J=1.9 Hz, 1H), 4.48 (d, J=6.0 Hz, 2H), 3.90 (s, 3H), 2.17 (s, 3H).

ESI-MS: 511.10 (M+H)⁺.

### N-[(3,5-difluorophenyl)methyl]-2-methyl-3-({2-[4-(1-methylpyrazol-3-yl)phenyl]-4-pyridyl}oxy)benzamide (283)

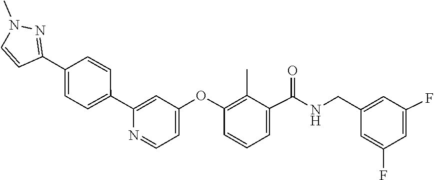 [SMILES: Cc1c(Oc2ccnc(-c3ccc(-c4ccn(C)n4)cc3)c2)cccc1C(=O)NCc1cc(F)cc(F)c1]

Compound 283 was synthesized in a two steps procedure from intermediate 199b (0.10 mmol), (4-bromophenyl) boronic acid (0.15 mmol) and 1-methyl-3-(4,4,5,5-tetramethyl-1,3,2-dioxaborolan-2-yl)-1H-pyrazole (0.21 mmol) as a white solid in 4% yield according to the general method D2. ¹H NMR (400 MHz, DMSO-d₆) δ (ppm): 9.01 (t, J=6.1 Hz, 1H), 8.53 (d, J=5.6 Hz, 1H), 8.06 (d, J=8.6 Hz, 2H), 7.88 (d, J=8.5 Hz, 2H), 7.76 (d, J=2.2 Hz, 1H), 7.53 (d, J=2.3 Hz, 1H), 7.43-7.37 (m, 2H), 7.28 (dd, J=7.4 Hz, 1.8 Hz, 1H), 7.15-7.10 (m, 1H), 7.09-. 704 (m, 2H), 6.76 (d, J=2.3 Hz, 1H), 6.69 (dd, J=5.6 Hz, 2.4 Hz, 1H), 4.48 (d, J=6.0 Hz, 2H), 3.90 (s, 3H), 2.17 (s, 3H).

ESI-MS: 511.10 (M+H)⁺.

### Example 11: General Procedure for the Synthesis of Analogues 285-290

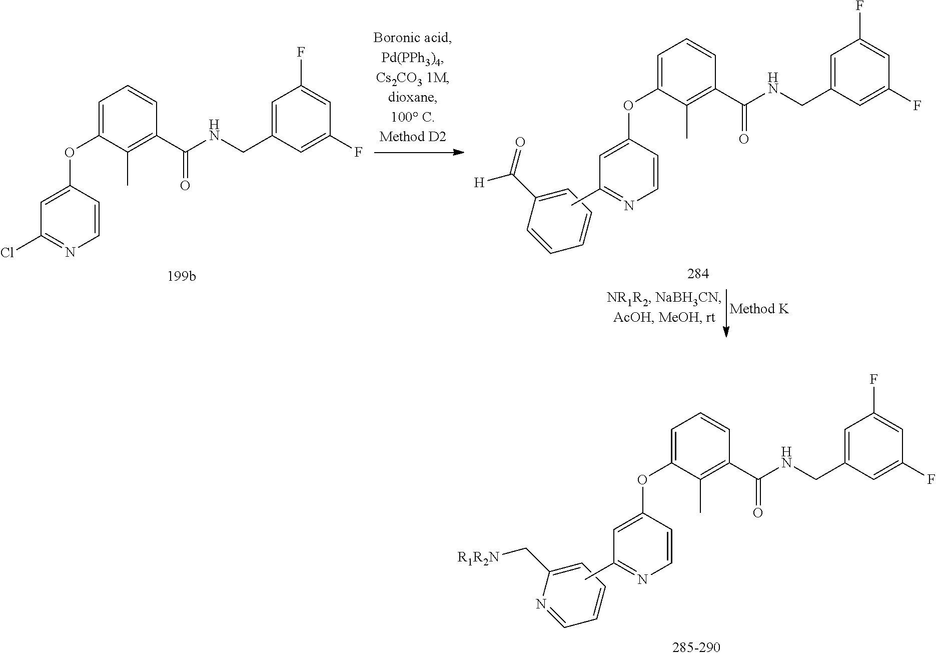

### Preparation of N-[(3,5-difluorophenyl)methyl]-3-{[2-(4-formylphenyl)-4-pyridyl]oxy}-2-methyl-benzamide (284a)

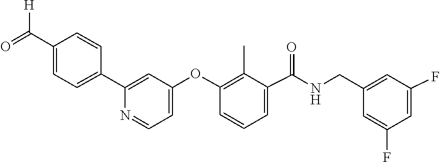 [SMILES: Cc1c(Oc2ccnc(-c3ccc(C=O)cc3)c2)cccc1C(=O)NCc1cc(F)cc(F)c1]

Intermediate 284a was synthesized from 199b (0.39 mmol) as a white solid in quantitative yield according to the general method D2.

ESI-MS: 459.05 (M+H)⁺.

The following table illustrates intermediates 284 prepared from Method D2.

| | | Synthesis |
|---|---|---|
| Intermediate | Structure | procedure |
| Compound 284a | 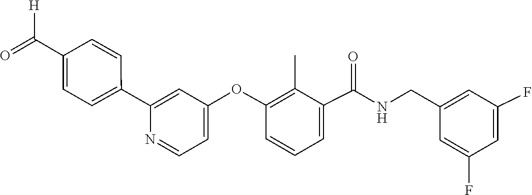 [SMILES: Cc1c(Oc2ccnc(-c3ccc(C=O)cc3)c2)cccc1C(=O)NCc1cc(F)cc(F)c1] | Method D2 |
| Compound 284b | 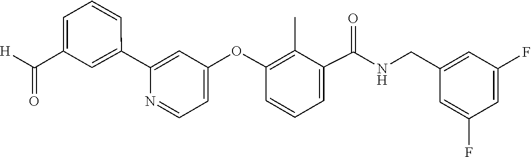 [SMILES: Cc1c(Oc2ccnc(-c3cccc(C=O)c3)c2)cccc1C(=O)NCc1cc(F)cc(F)c1] | Method D2 |

Method K: To a solution of 284 (1 equiv.) in MeOH (10 mL/mmol) were added amine derivative (1.3 equiv.), AcOH (2% v/v) and NaBH₃CN. The mixture was stirred at room temperature until completion (from 1 h to overnight). The reaction mixture was concentrated under reduced pressure. The crude was purified by flash column chromatography (DCM/MeOH from 100/0 to 90/10) and reverse phase chromatography (H₂O/MeOH from 100/0 to 0/100) to give the expected compound.

### N-[(3,5-difluorophenyl)methyl]-2-methyl-3-({2-[4-(pyrrolidin-1-ylmethyl)phenyl]-4-pyridyl}oxy)benzamide (285)

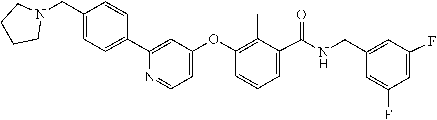 [SMILES: Cc1c(Oc2ccnc(-c3ccc(CN4CCCC4)cc3)c2)cccc1C(=O)NCc1cc(F)cc(F)c1]

Compound 285 was synthesized from intermediate 284a (0.05 mmol) and pyrrolidine (0.07 mmol) as a white solid in 25% yield according to the general method K. ¹H NMR (400 MHz, DMSO-d₆) δ (ppm): 9.01 (t, J=6.0 Hz, 1H), 8.52 (d, J=5.6 Hz, 1H), 7.96 (d, J=8.2 Hz, 2H), 7.45 (d, J=2.2 Hz, 1H), 7.43-7.37 (m, 4H), 7.27 (dd, J=7.3 Hz, 1.9 Hz, 1H), 7.15-7.04 (m, 3H), 6.69 (dd, J=5.6 Hz, 2.3 Hz, 1H), 4.48 (d, J=5.9 Hz, 2H), 3.61 (s, 2H), 2.43 (bs, 4H), 2.16 (s, 3H), 1.70 (bs, 4H).

ESI-MS: 514.15 (M+H)⁺.

### N-[(3,5-difluorophenyl)methyl]-2-methyl-3-({2-[4-(morpholinomethyl)phenyl]-4-pyridyl}oxy)benzamide (286)

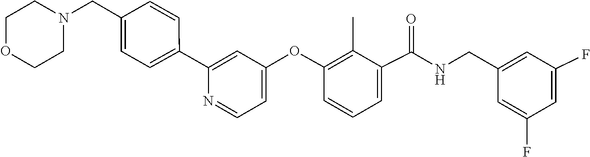 [SMILES: Cc1c(Oc2ccnc(-c3ccc(CN4CCOCC4)cc3)c2)cccc1C(=O)NCc1cc(F)cc(F)c1]

Compound 286 was synthesized from intermediate 284a (0.07 mmol) and morpholine (0.09 mmol) as a white solid in 26% yield according to the general method K. ¹H NMR (400 MHz, DMSO-d₆) δ (ppm): 9.01 (t, J=6.0 Hz, 1H), 8.52 (d, J=5.7 Hz, 1H), 7.97 (d, J=8.3 Hz, 2H), 7.46 (d, J=2.3 Hz, 1H), 7.41-7.37 (m, 4H), 7.27 (dd, J=7.4 Hz, 1.9 Hz, 1H), 7.13 (tt, J=9.5 Hz, 2.4 Hz, 1H), 7.09-7.04 (m, 2H), 6.69 (dd, J=5.6 Hz, 2.4 Hz, 1H), 4.48 (d, J=6.0 Hz, 2H), 3.59-3.56 (m, 4H), 3.51 (s, 2H), 2.38-2.35 (m, 4H), 2.15 (s, 3H).

ESI-MS: 530.15 (M+H)⁺.

### N-[(3,5-difluorophenyl)methyl]-2-methyl-3-[(2-{4-[(4-methylpiperazin-1-yl)methyl]phenyl}-4-pyridyl)oxy] benzamide (287)

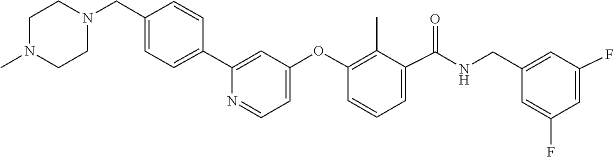 [SMILES: Cc1c(Oc2ccnc(-c3ccc(CN4CCN(C)CC4)cc3)c2)cccc1C(=O)NCc1cc(F)cc(F)c1]

Compound 287 was synthesized from intermediate 284a (0.07 mmol) and 1-methylpiperazine (0.09 mmol) as a white solid in 31% yield according to the general method K. ¹H NMR (400 MHz, DMSO-d₆) δ (ppm): 9.01 (t, J=6.1 Hz, 1H), 8.52 (d, J=5.7 Hz, 1H), 7.96 (d, J=8.3 Hz, 2H), 7.45 (d, J=2.3 Hz, 1H), 7.43-7.37 (m, 4H), 7.27 (dd, J=7.4 Hz, 1.9 Hz, 1H), 7.16-7.10 (m, 1H), 7.09-7.04 (m, 2H), 6.69 (dd, J=5.6 Hz, 2.4 Hz, 1H), 4.48 (d, J=6.0 Hz, 2H), 3.49 (s, 2H), 2.56-2.50 (m, 4H), 2.40-2.30 (m, 4H), 2.15 (s, 3H), 2.14 (s, 3H).

ESI-MS: 543.20 (M+H)⁺.

### N-[(3,5-difluorophenyl)methyl]-2-methyl-3-({2-[3-(pyrrolidin-1-ylmethyl)phenyl]-4-pyridyl}oxy)benzamide (288)

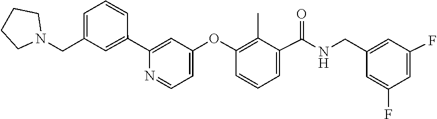 [SMILES: Cc1c(Oc2ccnc(-c3cccc(CN4CCCC4)c3)c2)cccc1C(=O)NCc1cc(F)cc(F)c1]

Compound 288 was synthesized from intermediate 284b (0.06 mmol) and pyrrolidine (0.09 mmol) as a white solid in 44% yield according to the general method K. ¹H NMR (400 MHz, DMSO-d₆) δ (ppm): 9.01 (t, J=6.0 Hz, 1H), 8.53 (d, J=5.7 Hz, 1H), 8.04 (s, 1H), 7.90 (s, 1H), 7.50-7.37 (m, 5H), 7.27 (dd, J=7.4 Hz, 1.9 Hz, 1H), 7.13 (tt, J=9.4 Hz, 2.4 Hz, 1H), 7.09-7.04 (m, 2H), 6.70 (dd, J=5.6 Hz, 2.4 Hz, 1H), 4.48 (d, J=6.0 Hz, 2H), 3.69 (s, 2H), 2.52 (bs, 4H), 2.16 (s, 3H), 1.74 (bs, 4H).

ESI-MS: 514.15 (M+H)⁺.

### N-[(3,5-difluorophenyl)methyl]-2-methyl-3-({2-[3-(morpholinomethyl)phenyl]-4-pyridyl}oxy)benzamide (289)

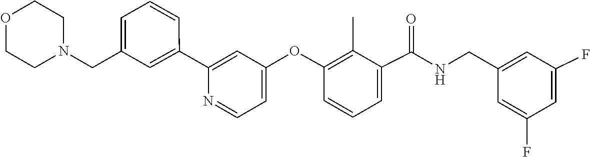 [SMILES: Cc1c(Oc2ccnc(-c3cccc(CN4CCOCC4)c3)c2)cccc1C(=O)NCc1cc(F)cc(F)c1]

Compound 289 was synthesized from intermediate 284b (0.07 mmol) and morpholine (0.09 mmol) as a white solid in 40% yield according to the general method K. ¹H NMR (400 MHz, DMSO-d₆) δ (ppm): 9.01 (t, J=6.0 Hz, 1H), 8.53 (d, J=5.7 Hz, 1H), 7.99 (bs, 1H), 7.88-7.86 (m, 1H), 7.48 (d, J=2.2 Hz, 1H), 7.45-7.37 (m, 4H), 7.27 (dd, J=7.4 Hz, 1.9 Hz, 1H), 7.16-7.10 (m, 1H), 7.09-7.04 (m, 2H), 6.70 (dd, J=5.6 Hz, 2.4 Hz, 1H), 4.48 (d, J=6.0 Hz, 2H), 3.58-3.56 (m, 4H), 3.53 (s, 2H), 2.38-2.36 (s, 4H), 2.16 (s, 3H).

ESI-MS: 530.15 (M+H)⁺.

### N-[(3,5-difluorophenyl)methyl]-2-methyl-3-[(2-{3-[(4-methylpiperazin-1-yl)methyl]phenyl}-4-pyridyl)oxy]benzamide (290)

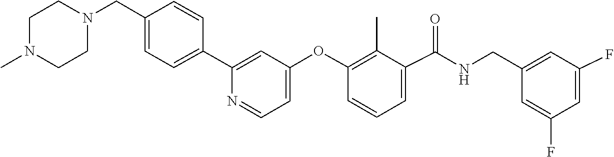 [SMILES: Cc1c(Oc2ccnc(-c3ccc(CN4CCN(C)CC4)cc3)c2)cccc1C(=O)NCc1cc(F)cc(F)c1]

Compound 290 was synthesized from intermediate 284b (0.07 mmol) and 1-methylpiperazine (0.09 mmol) as a white solid in 36% yield according to the general method K. ¹H NMR (400 MHz, DMSO-d₆) δ (ppm): 9.01 (t, J=6.1 Hz, 1H), 8.53 (d, J=5.7 Hz, 1H), 7.97 (s, 1H), 7.88-7.84 (m, 1H), 7.46 (d, J=2.2 Hz, 1H), 7.44-7.34 (m, 4H), 7.27 (dd, J=7.5 Hz, 1.8 Hz, 1H), 7.13 (tt, J=9.4 Hz, 2.4 Hz, 1H), 7.09-7.04 (m, 2H), 6.70 (dd, J=5.6 Hz, 2.4 Hz, 1H), 4.48 (d, J=6.0 Hz, 2H), 3.51 (s, 2H), 2.52-2.50 (m, 4H), 2.37-2.30 (m, 4H), 2.16 (s, 3H), 2.14 (s, 3H).

ESI-MS: 543.20 (M+H)⁺.

### Example 12: General Procedure for the Synthesis of Analogues 292-297

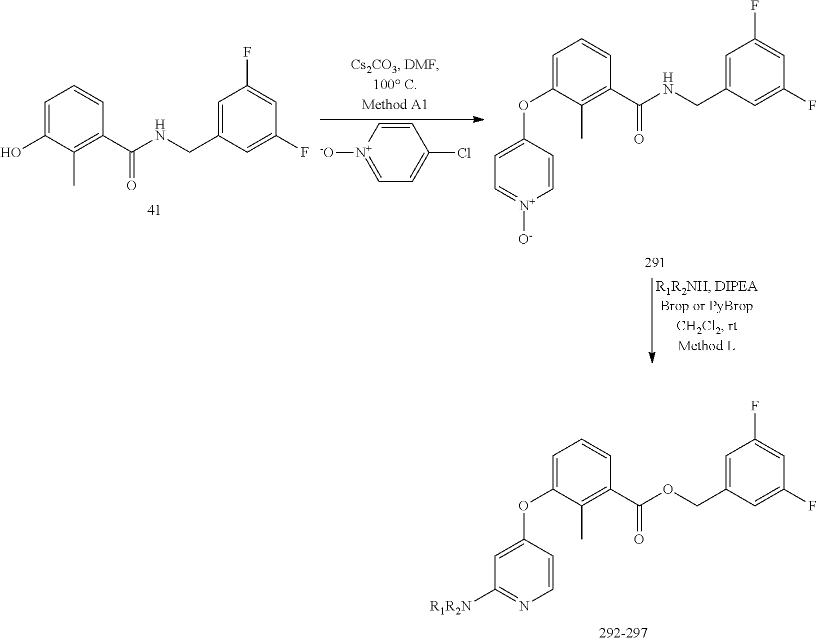

### Preparation of N-[(3,5-difluorophenyl)methyl]-2-methyl-3-(1-oxidopyridin-1-ium-4-yl)oxy-benzamide (291)

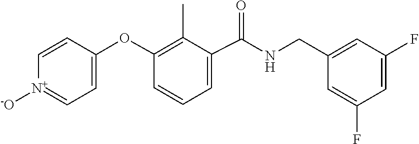

Intermediate 291 was synthesized from 41 (0.77 mmol) and 4-chloropyridine-N-oxide (0.77 mmol) as a white solid in 59% yield according to the general method A.

ESI-MS: 371.05 (M+H)⁺.
Method L: To a solution of 291 (1 equiv.) in CH₂Cl₂ (10 mL/mmol) were added amine derivative (1.3 equiv.), DIPEA (3.8 equiv.) and Brop or PyBrop (1.3 equiv.). The mixture was stirred at room temperature until completion (from 1 h to overnight). The reaction mixture was diluted with DCM and washed twice with a saturated solution of NaHCO₃. The organic layer was dried over Na₂SO₄, filtered and concentrated under reduced pressure. The crude was purified by flash column chromatography (DCM/MeOH from 100/0 to 90/10) and reverse phase chromatography (H₂O/MeOH from 100/0 to 0/100) to give the expected compound.

The following compounds are examples illustrating Method L:

### N-[(3,5-difluorophenyl)methyl]-2-methyl-3-{[2-(methylamino)-4-pyridyl]oxy} benzamide (292)

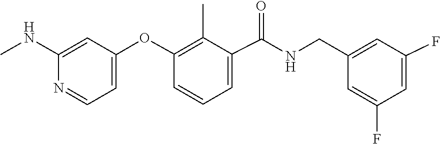 [SMILES: CNc1cc(Oc2cccc(C(=O)NCc3cc(F)cc(F)c3)c2C)ccn1]

Compound 292 was synthesized from intermediate 291 (0.08 mmol) and methylamine (2M THF, 0.10 mmol) as a white solid in 32% yield according to the general method L. ¹H NMR (400 MHz, DMSO-d₆) δ (ppm): 8.99 (t, J=6.0 Hz, 1H), 7.87 (d, J=5.8 Hz, 1H), 7.37-7.30 (m, 2H), 7.17-7.03 (m, 4H), 6.47 (q, J=4.7 Hz, 1H), 6.06 (dd, J=5.8 Hz, 2.2 Hz, 1H), 5.75 (d, J=2.2 Hz, 1H), 4.46 (d, J=6.0 Hz, 2H), 2.70 (d, J=4.8 Hz, 3H), 2.11 (s, 3H).

ESI-MS: 384.10 (M+H)⁺.

### 3-{[2-(cyclopropylamino)-4-pyridyl]oxy}-N-[(3,5-difluorophenyl)methyl]-2-methyl-benzamide (293)

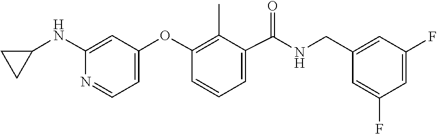 [SMILES: Cc1c(Oc2ccnc(NC3CC3)c2)cccc1C(=O)NCc1cc(F)cc(F)c1]

Compound 293 was synthesized from intermediate 291 (0.08 mmol) and cyclopropylamine (0.11 mmol) as a white solid in 30% yield according to the general method L. ¹H NMR (400 MHz, DMSO-d₆) δ (ppm): 8.99 (t, J=6.0 Hz, 1H), 7.87 (d, J=5.7 Hz, 1H), 7.38-7.31 (m, 2H), 7.18-7.10 (m, 2H), 7.09-7.03 (m, 2H), 6.82 (d, J=2.0 Hz, 1H), 6.04 (dd, J=5.7 Hz, 2.1 Hz, 1H), 5.97 (d, J=1.9 Hz, 1H), 4.46 (d, J=5.9 Hz, 2H), 2.40-2.43 (m, 1H), 2.12 (s, 3H), 0.64-0.59 (m, 2H), 0.38-0.34 (m, 2H).

ESI-MS: 410.10 (M+H)⁺.

### 3-[(2-anilino-4-pyridyl)oxy]-N-[(3,5-difluorophenyl)methyl]-2-methyl-benzamide (294)

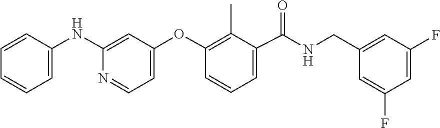 [SMILES: Cc1c(Oc2ccnc(Nc3ccccc3)c2)cccc1C(=O)NCc1cc(F)cc(F)c1]

Compound 294 was synthesized from intermediate 291 (0.05 mmol) and aniline (0.11 mmol) as a white solid in 13% yield according to the general method L. 1H NMR (400 MHz, DMSO-d₆) δ (ppm): 8.98 (s, 2H), 8.05 (d, J=5.8 Hz, 1H), 7.61 (d, J=8.0 Hz, 2H), 7.41-7.35 (m, 2H), 7.24-7.05 (m, 6H), 6.86 (t, J=7.3 Hz, 1H), 6.39-6.38 (m, 1H), 6.13 (s, 1H), 4.48 (d, J=5.8 Hz, 2H), 2.15 (s, 3H).

ESI-MS: 446.10 (M+H)⁺.

### N-[(3,5-difluorophenyl)methyl]-2-methyl-3-{[2-(3-pyridylamino)-4-pyridyl]oxy} benzamide (295)

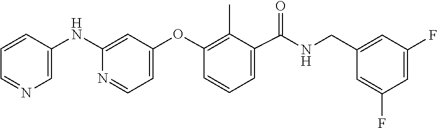 [SMILES: Cc1c(Oc2ccnc(Nc3cccnc3)c2)cccc1C(=O)NCc1cc(F)cc(F)c1]

Compound 295 was synthesized from intermediate 291 (0.05 mmol) and 3-aminopyridine (0.27 mmol) as a white solid in 37% yield according to the general method L. ¹H NMR (400 MHz, DMSO-d₆) δ (ppm): 9.20 (s, 1H), 8.99 (t, J=5.9 Hz, 1H), 8.72 (d, J=2.5 Hz, 1H), 8.17 (ddd, J=8.4 Hz, 2.6 Hz, 1.5 Hz, 1H), 8.10-8.06 (m, 2H), 7.43-7.36 (m, 2H), 7.26-7.23 (m, 2H), 7.13 (tt, J=9.4 Hz, 2.3 Hz, 1H), 7.09-7.04 (m, 2H), 6.46 (dd, J=5.8 Hz, 2.2 Hz, 1H), 6.13 (d, J=2.2 Hz, 1H), 4.48 (d, J=6.0 Hz, 2H), 2.15 (s, 3H).

ESI-MS: 447.10 (M+H)⁺.

### N-[(3,5-difluorophenyl)methyl]-2-methyl-3-{[2-(4-methylpyrazol-1-yl)-4-pyridyl] oxy}benzamide (296)

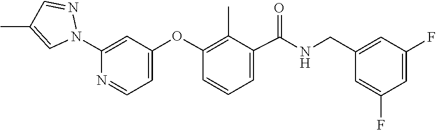 [SMILES: Cc1cnn(-c2cc(Oc3cccc(C(=O)NCc4cc(F)cc(F)c4)c3C)ccn2)c1]

Compound 296 was synthesized from intermediate 291 (0.07 mmol) and 4-methylpyrazole (0.34 mmol) as a white solid in 38% yield according to the general method L. ¹H NMR (400 MHz, DMSO-d₆) δ (ppm): 9.08 (t, J=6.0 Hz, 1H), 8.37 (s, 1H), 8.34 (d, J=5.7 Hz, 1H), 7.57 (s, 1H), 7.45-7.40 (m, 2H), 7.31 (dd, J=7.0 Hz, 2.3 Hz, 1H), 7.16-7.09 (m, 2H), 7.09-7.04 (m, 2H), 6.90 (dd, J=5.7 Hz, 2.4 Hz, 1H), 4.47 (d, J=6.0 Hz, 2H), 2.14 (s, 3H), 2.08 (s, 3H).

ESI-MS: 435.10 (M+H)⁺.

### N-[(3,5-difluorophenyl)methyl]-2-methyl-3-{[2-(3-methylpyrazol-1-yl)-4-pyridyl] oxy}benzamide (297)

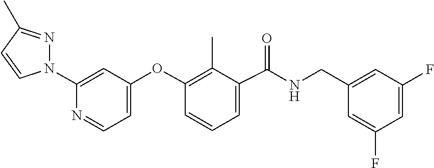 [SMILES: Cc1ccn(-c2cc(Oc3cccc(C(=O)NCc4cc(F)cc(F)c4)c3C)ccn2)n1]

Compound 297 was synthesized from intermediate 291 (0.16 mmol) and 3-methylpyrazole (0.78 mmol) as a white solid in 15% yield according to the general method L. ¹H NMR (400 MHz, DMSO-d₆) δ (ppm): 9.06 (t, J=6.0 Hz, 1H), 8.47 (d, J=2.4 Hz, 1H), 8.33 (d, J=5.7 Hz, 1H), 7.46-7.40 (m, 2H), 7.31 (dd, J=7.3 Hz, 2.0 Hz, 1H), 7.16-7.09 (m, 2H), 7.08-7.04 (m, 2H), 6.88 (dd, J=5.7 Hz, 2.4 Hz, 1H), 6.35 (d, J=2.5 Hz, 1H), 4.48 (d, J=6.0 Hz, 2H), 2.22 (s, 3H), 2.15 (s, 3H).

ESI-MS: 435.20 (M+H)⁺.

### Example 13: General Procedure for the Synthesis of Analogues 302-309

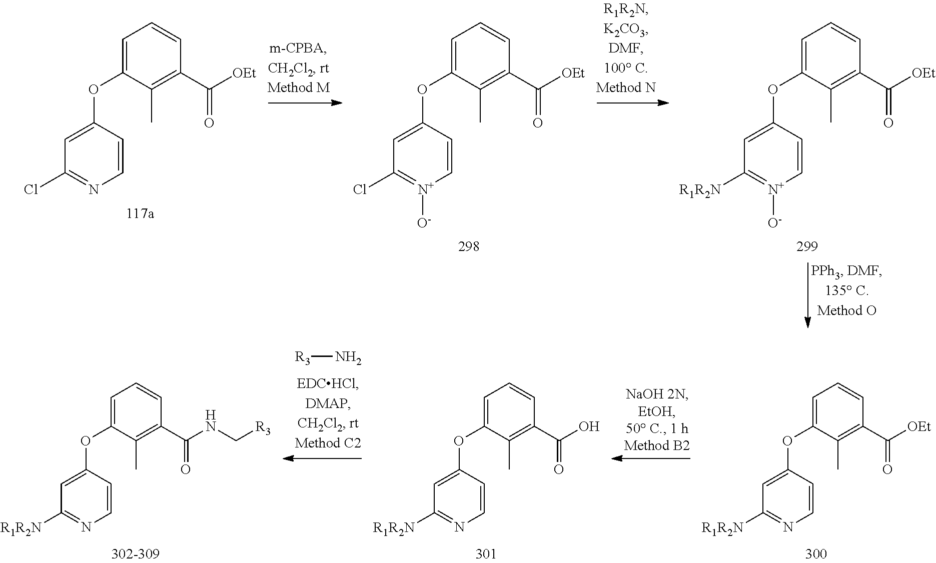

Method M: To a stirred solution of 117a (400 mg, 1.37 mmol) in CH₂Cl₂ (6 mL/mmol) was added m-CPBA (77%, 922 mg, 4.11 mmol) and the reaction mixture was stirred at room temperature for 24 h. The reaction mixture was diluted with DCM and washed twice with a saturated solution of NaHCO₃. The organic layer was dried over Na₂SO₄, filtered and concentrated under reduced pressure. The crude was purified by flash column chromatography (CH₂Cl₂/MeOH from 100/0 to 93/7) to give 356 mg of ethyl 3-(2-chloro-1-oxido-pyridin-1-ium-4-yl)oxy-2-methyl-benzoate 298 in 85% yield as yellow oil. ESI-MS: 308.00 (M+H)⁺.
Method N: To a stirred solution of 298 (1 equiv.) in DMF (10 mL/mmol) were added amine derivative (1 to 3 equiv.) and K₂CO₃ (1.2 equiv.). The reaction mixture was stirred at 100° C. overnight. The reaction mixture was concentrated under reduced pressure. The crude was purified by flash column chromatography (DCM/MeOH from 100/0 to 94/6) to give the expected compounds.

The following compound 299 is an example illustrating Method N: Preparation of ethyl 2-methyl-3-[1-oxido-2-(triazol-2-yl)pyridin-1-ium-4-yl]oxy-benzoate (299a) and ethyl 2-methyl-3-[1-oxido-2-(triazol-1-yl)pyridin-1-ium-4-yl]oxy-benzoate (299b)

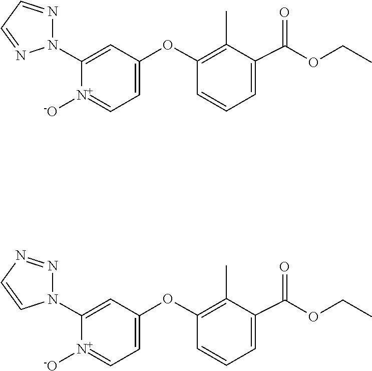

Intermediate 299a and 299b were synthesized from 298 (1.02 mmol) and 1H-1,2,3-triazole (6.02 mmol) as a white solid in 33% and 23% yield respectively according to the general method N.

ESI-MS: 341.00 (M+H)⁺.

The following table illustrates intermediates 299 prepared from method N:

| | | Synthesis |
|---|---|---|
| Intermediate | Structure | procedure |
| Compound 299a | 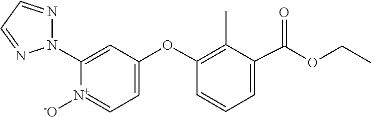 | Method N |
| Compound 299b | 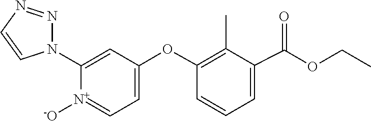 | Method N |
| Compound 299c | 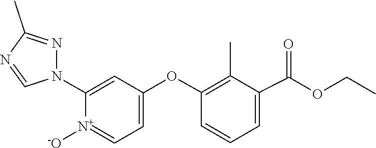 | Method N |
| Compound 299d | 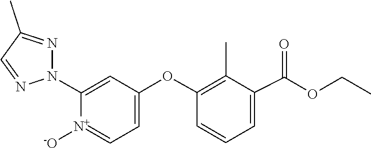 | Method N |
| Compound 299e | 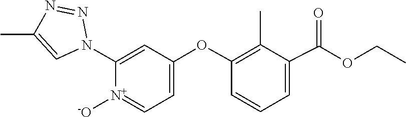 | Method N |

Method O: To a stirred solution of 299 (1 equiv.) in DMF (10 mL/mmol) was added PPh₃ (2 equiv.) and the reaction mixture was stirred at 135° C. overnight. The reaction mixture was concentrated under reduced pressure. The crude was purified by flash column chromatography (DCM/MeOH from 100/0 to 96/4) to give the expected compound.

### Preparation of ethyl 2-methyl-3-{[2-(triazol-2-yl)-4-pyridyl]oxy} benzoate (300a)

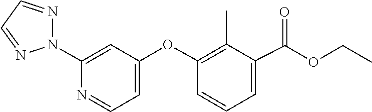 [SMILES: CCOC(=O)c1cccc(Oc2ccnc(-n3nccn3)c2)c1C]

Intermediate 300a was synthesized from 299a (0.32 mmol) and PPh₃ (0.64 mmol) as a white solid in 70% yield according to the general method O. ¹H NMR (400 MHz, DMSO-d₆) δ (ppm): 8.47 (d, J=5.7 Hz, 1H), 8.14 (s, 2H), 7.81-7.76 (m, 1H), 7.50-7.45 (m, 2H), 7.30 (d, J=2.2 Hz, 1H), 7.03 (dd, J=5.7 Hz, 2.3 Hz, 1H), 4.33 (q, J=7.1 Hz, 2H), 2.32 (s, 3H), 1.33 (t, J=7.1 Hz, 3H).

ESI-MS: 325.05 (M+H)⁺.

The following table illustrates intermediates 300 prepared from method O:

| | | Synthesis |
|---|---|---|
| Intermediate | Structure | procedure |
| Compound 300a | 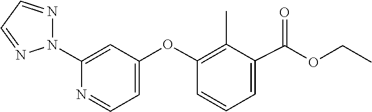 [SMILES: CCOC(=O)c1cccc(Oc2ccnc(-n3nccn3)c2)c1C] | Method O |
| Compound 300b | 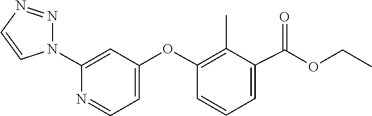 [SMILES: CCOC(=O)c1cccc(Oc2ccnc(-n3ccnn3)c2)c1C] | Method O |
| Compound 300c | 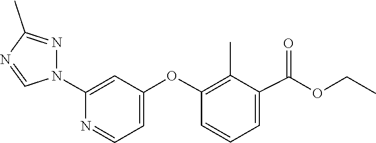 [SMILES: CCOC(=O)c1cccc(Oc2ccnc(-n3cnc(C)n3)c2)c1C] | Method O |
| Compound 300d | 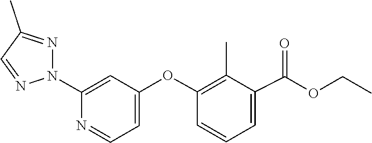 [SMILES: CCOC(=O)c1cccc(Oc2ccnc(-n3ncc(C)n3)c2)c1C] | Method O |
| Compound 300e | 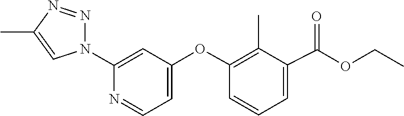 [SMILES: CCOC(=O)c1cccc(Oc2ccnc(-n3cc(C)nn3)c2)c1C] | Method O |

The following table illustrates intermediates 301 prepared from method B2:

| Intermediate | Structure | Synthesis procedure |
|---|---|---|
| Compound 301a | 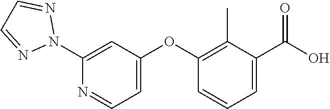 [SMILES: Cc1c(Oc2ccnc(-n3nccn3)c2)cccc1C(=O)O] | Method B2 |
| Compound 301b | 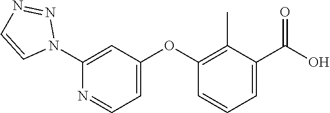 [SMILES: Cc1c(Oc2ccnc(-n3ccnn3)c2)cccc1C(=O)O] | Method B2 |
| Compound 301c | 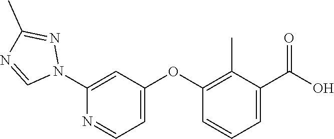 [SMILES: Cc1ncn(-c2cc(Oc3cccc(C(=O)O)c3C)ccn2)n1] | Method B2 |
| Compound 301d | 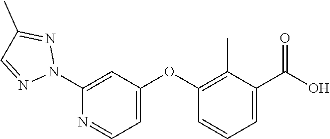 [SMILES: Cc1cnn(-c2cc(Oc3cccc(C(=O)O)c3C)ccn2)n1] | Method B2 |
| Compound 301e | 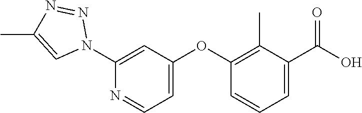 [SMILES: Cc1cn(-c2cc(Oc3cccc(C(=O)O)c3C)ccn2)nn1] | Method B2 |

The following compounds are examples illustrating Method C2:

### N-[(6-methoxy-3-pyridyl)methyl]-2-methyl-3-{[2-(triazol-2-yl)-4-pyridyl]oxy} benzamide (302)

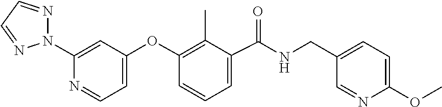 [SMILES: COc1ccc(CNC(=O)c2cccc(Oc3ccnc(-n4nccn4)c3)c2C)cn1]

Compound 302 was synthesized from intermediate 301a (0.07 mmol) and 6-methoxypyridin-3-yl) methanamine (0.10 mmol) as a white solid in 79% yield according to the general method C2. ¹H NMR (400 MHz, DMSO-d₆) δ (ppm): 8.95 (t, J=5.9 Hz, 1H), 8.47 (d, J=5.7 Hz, 1H), 8.14 (s, 3H), 7.69 (dd, J=8.5 Hz, 2.5 Hz, 1H), 7.41 (t, J=7.8 Hz, 1H), 7.36-7.30 (m, 3H), 7.03 (dd, J=5.7 Hz, 2.3 Hz, 1H), 6.81 (d, J=8.5 Hz, 1H), 4.39 (d, J=5.9 Hz, 2H), 3.83 (s, 3H), 2.12 (s, 3H).

ESI-MS: 417.05 (M+H)⁺.

### N-[(3,5-difluorophenyl)methyl]-2-methyl-3-{[2-(triazol-2-yl)-4-pyridyl]oxy} benzamide (303)

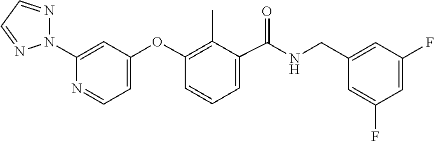 [SMILES: Cc1c(Oc2ccnc(-n3nccn3)c2)cccc1C(=O)NCc1cc(F)cc(F)c1]

Compound 303 was synthesized from intermediate 301a (0.07 mmol) and 3,5-difluorobenzylamine (0.10 mmol) as a white solid in 82% yield according to the general method C2. ¹H NMR (400 MHz, DMSO-d₆) δ (ppm): 9.05 (t, J=6.1 Hz, 1H), 8.47 (d, J=5.7 Hz, 1H), 8.14 (s, 2H), 7.46-7.41 (m, 2H), 7.34 (dd, J=7.0 Hz, 2.4 Hz, 1H), 7.32 (d, J=2.2 Hz, 1H), 7.15-7.09 (m, 1H), 7.09-7.03 (m, 3H), 4.48 (d, J=6.0 Hz, 2H), 2.15 (s, 3H).

ESI-MS: 422.05 (M+H)⁺.

### N-[(6-methoxy-3-pyridyl)methyl]-2-methyl-3-{[2-(triazol-1-yl)-4-pyridyl]oxy} benzamide (304)

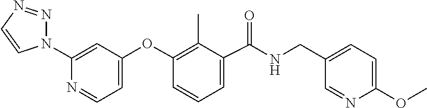 [SMILES: COc1ccc(CNC(=O)c2cccc(Oc3ccnc(-n4ccnn4)c3)c2C)cn1]

Compound 304 was synthesized from intermediate 301b (0.07 mmol) and 6-methoxypyridin-3-yl) methanamine (0.10 mmol) as a white solid in 68% yield according to the general method C2. ¹H NMR (400 MHz, DMSO-d₆) δ (ppm): 8.97 (t, J=5.9 Hz, 1H), 8.84 (d, J=1.2 Hz, 1H), 8.49 (d, J=5.8 Hz, 1H), 8.14 (d, J=2.0 Hz, 1H), 7.97 (d, J=1.2 Hz, 1H), 7.70 (dd, J=8.5 Hz, 2.5 Hz, 1H), 7.45-7.40 (m, 2H), 7.37-7.32 (m, 2H), 7.07 (dd, J=5.8 Hz, 2.3 Hz, 1H), 6.81 (d, J=8.5 Hz, 1H), 4.39 (d, J=5.9 Hz, 2H), 3.83 (s, 3H), 2.13 (s, 3H).

ESI-MS: 417.05 (M+H)⁺.

### N-[(3,5-difluorophenyl)methyl]-2-methyl-3-{[2-(triazol-1-yl)-4-pyridyl]oxy} benzamide (305)

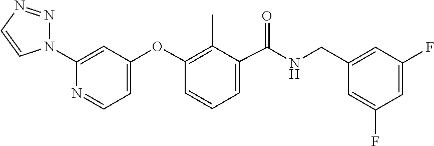 [SMILES: Cc1c(Oc2ccnc(-n3ccnn3)c2)cccc1C(=O)NCc1cc(F)cc(F)c1]

Compound 305 was synthesized from intermediate 301b (0.07 mmol) and 3,5-difluorobenzylamine (0.10 mmol) as a white solid in 64% yield according to the general method C2. ¹H NMR (400 MHz, DMSO-d₆) δ (ppm): 9.07 (t, J=6.1 Hz, 1H), 8.85 (d, J=1.2 Hz, 1H), 8.50 (d, J=5.8 Hz, 1H), 7.98 (d, J=1.2 Hz, 1H), 7.48-7.42 (m, 3H), 7.36 (dd, J=7.1 Hz, 2.3 Hz, 1H), 7.15-7.04 (m, 4H), 4.48 (d, J=6.0 Hz, 2H), 2.16 (s, 3H).

ESI-MS: 422.10 (M+H)⁺.

### N-[(3,5-difluorophenyl)methyl]-2-methyl-3-{[2-(3-methyl-1,2,4-triazol-1-yl)-4-pyridyl]oxy}benzamide (306)

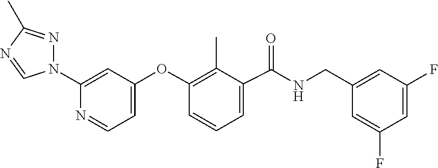 [SMILES: Cc1ncn(-c2cc(Oc3cccc(C(=O)NCc4cc(F)cc(F)c4)c3C)ccn2)n1]

Compound 306 was synthesized from intermediate 301c (0.06 mmol) and 3,5-difluorobenzylamine (0.10 mmol) as a white solid in 65% yield according to the general method C2. ¹H NMR (400 MHz, DMSO-d₆) δ (ppm): 9.21 (s, 1H), 9.07 (t, J=6.0 Hz, 1H), 8.42 (d, J=5.7 Hz, 1H), 7.47-7.42 (m, 2H), 7.33 (dd, J=6.9 Hz, 2.4 Hz, 1H), 7.13 (tt, J=9.4 Hz, 2.3 Hz, 1H), 7.09-7.05 (m, 3H), 7.01 (dd, J=5.7 Hz, 2.3 Hz, 1H), 4.48 (d, J=6.0 Hz, 2H), 2.32 (s, 3H), 2.14 (s, 3H).

ESI-MS: 436.15 (M+H)⁺.

### N-[(6-methoxy-3-pyridyl)methyl]-2-methyl-3-{[2-(3-methyl-1,2,4-triazol-1-yl)-4-pyridyl]oxy}benzamide (307)

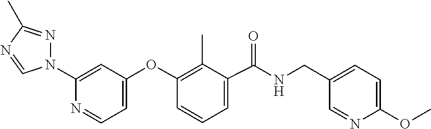 [SMILES: COc1ccc(CNC(=O)c2cccc(Oc3ccnc(-n4cnc(C)n4)c3)c2C)cn1]

Compound 307 was synthesized from intermediate 301c (0.06 mmol) and 6-methoxypyridin-3-yl) methanamine (0.10 mmol) as a white solid in 54% yield according to the general method C2. ¹H NMR (400 MHz, DMSO-d₆) δ (ppm): 9.21 (s, 1H), 8.97 (t, J=5.9 Hz, 1H), 8.41 (d, J=5.7 Hz, 1H), 8.14 (d, J=2.2 Hz, 1H), 7.69 (dd, J=8.5 Hz, 2.4 Hz, 1H), 7.42 (t, J=7.7 Hz, 1H), 7.36-7.34 (m, 1H), 7.32-7.29 (m, 1H), 7.05 (d, J=2.3 Hz, 1H), 7.00 (dd, J=5.7 Hz, 2.3 Hz, 1H), 6.81 (d, J=8.5 Hz, 1H), 4.39 (d, J=5.9 Hz, 2H), 3.83 (s, 3H), 2.32 (s, 3H), 2.11 (s, 3H).

ESI-MS: 431.15 (M+H)⁺.

### N-[(3,5-difluorophenyl)methyl]-2-methyl-3-{[2-(4-methyltriazol-2-yl)-4-pyridyl]oxy}benzamide (308)

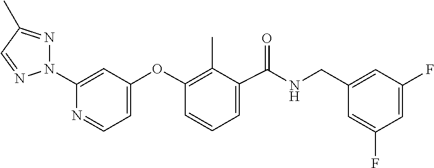 [SMILES: Cc1cnn(-c2cc(Oc3cccc(C(=O)NCc4cc(F)cc(F)c4)c3C)ccn2)n1]

Compound 308 was synthesized from intermediate 301d (0.11 mmol) and 3,5-difluorobenzylamine (0.16 mmol) as a white solid in 81% yield according to the general method C2. ¹H NMR (400 MHz, DMSO-d₆) δ (ppm): 9.07 (t, J=6.1 Hz, 1H), 8.43 (d, J=5.7 Hz, 1H), 7.91 (s, 1H), 7.47-7.40 (m, 2H), 7.34 (dd, J=7.0 Hz, 2.3 Hz, 1H), 7.23 (d, J=2.2 Hz, 1H), 7.13 (tt, J=9.4 Hz, 2.3 Hz, 1H), 7.09-7.04 (m, 2H), 7.01 (dd, J=5.7 Hz, 2.3 Hz, 1H), 4.47 (d, J=6.0 Hz, 2H), 2.33 (s, 3H), 2.14 (s, 3H).

ESI-MS: 436.10 (M+H)⁺.

### N-[(3,5-difluorophenyl)methyl]-2-methyl-3-{[2-(4-methyltriazol-1-yl)-4-pyridyl] oxy}benzamide (309)

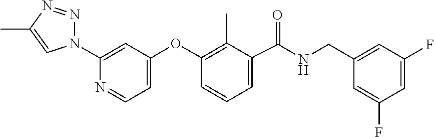 [SMILES: Cc1cn(-c2cc(Oc3cccc(C(=O)NCc4cc(F)cc(F)c4)c3C)ccn2)nn1]

Compound 309 was synthesized from intermediate 301e (0.11 mmol) and 3,5-difluorobenzylamine (0.16 mmol) as a white solid in 59% yield according to the general method C2. ¹H NMR (400 MHz, DMSO-d₆) δ (ppm): 9.09 (t, J=6.0 Hz, 1H), 8.58 (d, J=0.8 Hz, 1H), 8.47 (d, J=5.8 Hz, 1H), 7.47-7.41 (m, 2H), 7.37-7.34 (m, 2H), 7.13 (tt, J=9.5 Hz, 2.3 Hz, 1H), 7.09-7.04 (m, 3H), 4.48 (d, J=6.0 Hz, 2H), 2.32 (s, 3H), 2.15 (s, 3H).

ESI-MS: 436.15 (M+H)⁺.

### Example 14: General Procedure for the Synthesis of Analogues 310-314

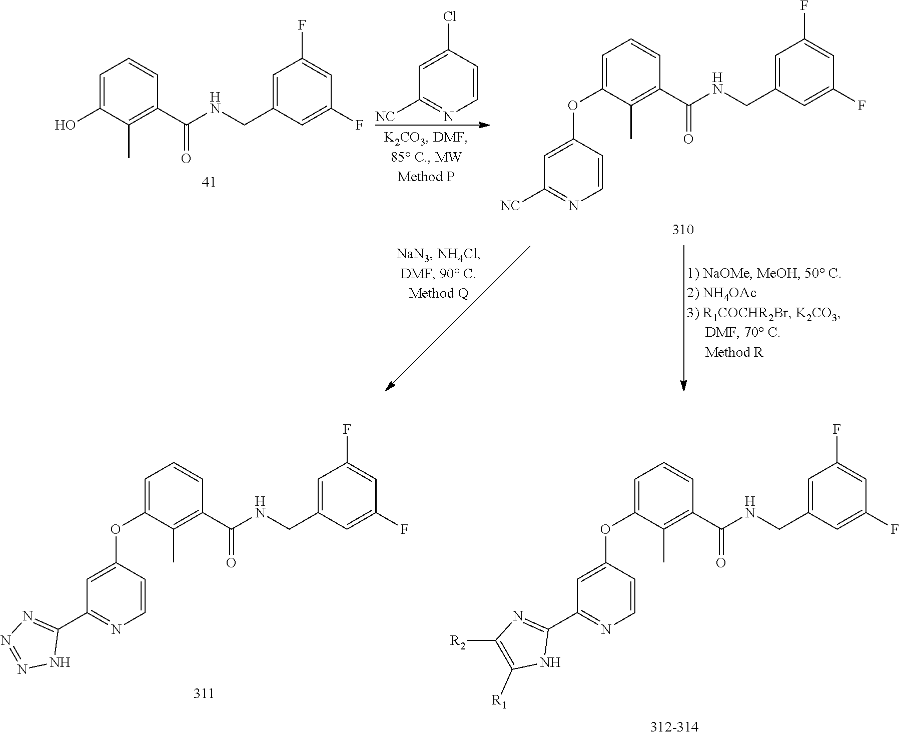

Method P: Compound 41 (515 mg, 1.86 mmol) and 4-chloropyridine-2-carbonitrile (198 mg, 1.43 mmol) were dissolved in DMF (10 mL/mmol) in an oven-dried screw-cap test tube. K2CO₃ (395 mg, 2.90 mmol) was added and the reaction mixture was stirred and heated under microwave irradiation at 85° C. for 8 h. The reaction mixture was diluted with EtOAc and washed twice with a saturated solution of NH₄Cl. The organic layer was dried over Na₂SO₄, filtered and concentrated under reduced pressure. The crude was purified by flash column chromatography (CH₂Cl₂/MeOH from 100/0 to 95/5) to give 321 mg of 3-[(2-cyano-4-pyridyl)oxy]-N-[(3,5-difluorophenyl)methyl]-2-methyl-benzamide 310 in 59% yield as white powder.

¹H NMR (400 MHz, DMSO-d₆) δ (ppm): 8.99 (t, J=6.0 Hz, 1H), 8.60 (dd, J=5.8 Hz, 0.4 Hz, 1H), 7.62 (dd, J=2.5 Hz, 0.4 Hz, 1H), 7.45-7.40 (m, 2H), 7.30-7.26 (m, 1H), 7.16-7.04 (m, 4H), 4.48 (d, J=6.0 Hz, 2H), 2.11 (s, 3H).

ESI-MS: 436.15 (M+H)⁺.
Method Q: To a stirred solution of 310 (20 mg, 0.05 mmol) in DMF (1 mL) were added NH₄Cl (6 mg, 0.11 mmol) and sodium azide (7 mg, 0.11 mmol). The reaction mixture was stirred at 90° C. overnight. The reaction mixture was diluted with DCM and washed twice with a saturated solution of NH₄Cl. The organic layer was dried over Na₂SO₄, filtered and concentrated under reduced pressure.

The crude was purified by flash column chromatography (CH₂Cl₂/MeOH from 100/0 to 80/20) and reverse phase chromatography (H₂O/MeOH from 100/0 to 0/100) to give 3 mg of N-[(3,5-difluorophenyl)methyl]-2-methyl-3-{[2-(1H-tetrazol-5-yl)-4-pyridyl]oxy}benzamide 311 in 14% yield as beige solid. ¹H NMR (400 MHz, DMSO-d₆) δ (ppm): 9.09 (t, J=6.0 Hz, 1H), 8.63 (d, J=5.7 Hz, 1H), 7.49 (d, J=2.3 Hz, 1H), 7.47-7.40 (m, 2H), 7.32 (dd, J=7.3 Hz, 2.0 Hz, 1H), 7.15-7.03 (m, 4H), 4.48 (d, J=6.0 Hz, 2H), 2.15 (s, 3H).

ESI-MS: 423.00 (M+H)⁺.
Method R: To a stirred solution of 310 (1 equiv.) in n-propanol (10 mL/mmol) was added NaOMe (25% in MeOH, 1.2 equiv.) and the reaction mixture was stirred at 50° C. for 1 h 30. Then, NH₄OAc was added and the reaction mixture was stirred at 70° C. for 1 h. The reaction mixture was diluted with EtOAc and washed twice with water and brine. The organic layer was dried over Na₂SO₄, filtered and concentrated under reduced pressure to give the amidine derivative with was used directly in the next step. Compound was dissolved in DMF (10 mL/mmol) and K2CO₃ (2 equiv.) and bromo derivative (1.2 to 2 equiv.) were added. The reaction mixture was stirred at 90° C. for 3 h. The reaction mixture was diluted with EtOAc and washed twice with a saturated solution of NH₄Cl. The organic layer was dried over Na₂SO₄, filtered and concentrated under reduced pressure. The crude was purified by flash column chromatography (CH₂Cl₂/MeOH from 100/0 to 90/10) and reverse phase chromatography (H₂O/MeOH from 100/0 to 0/100) to give the expected compound.

The following compounds are examples illustrating Method R:

### N-[(3,5-difluorophenyl)methyl]-3-{[2-(4,5-dimethyl-1H-imidazol-2-yl)-4-pyridyl] oxy}-2-methyl-benzamide (312)

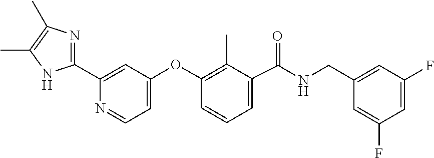 [SMILES: Cc1nc(-c2cc(Oc3cccc(C(=O)NCc4cc(F)cc(F)c4)c3C)ccn2)[nH]c1C]

Compound 312 was synthesized from intermediate 310 (0.07 mmol) and 3-bromo-2-butanone (0.13 mmol) as a white solid in 23% yield according to the general method R. ¹H NMR (400 MHz, DMSO-d₆) δ (ppm): 12.33 (bs, 1H), 9.07 (t, J=6.0 Hz, 1H), 8.43 (d, J=5.7 Hz, 1H), 7.45-7.38 (m, 2H), 7.28 (dd, J=7.6 Hz, 1.7 Hz, 1H), 7.16-7.04 (m, 4H), 6.91 (dd, J=5.7 Hz, 2.6 Hz, 1H), 4.47 (d, J=6.0 Hz, 2H), 2.14 (s, 3H), 2.12 (s, 3H), 2.01 (s, 3H).

ESI-MS: 449.10 (M+H)⁺.

### N-[(3,5-difluorophenyl)methyl]-2-methyl-3-({2-[5-(trifluoromethyl)-1H-imidazol-2-yl]-4-pyridyl}oxy)benzamide (313)

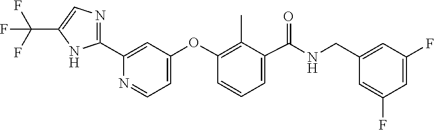 [SMILES: Cc1c(Oc2ccnc(-c3ncc(C(F)(F)F)[nH]3)c2)cccc1C(=O)NCc1cc(F)cc(F)c1]

Compound 313 was synthesized from intermediate 310 (0.05 mmol) and 3-bromo-1,1,1-trifluoroacetone (0.06 mmol) as a white solid in 23% yield according to the general method R. ¹H NMR (400 MHz, DMSO-d₆) δ (ppm): 13.47 (bs, 1H), 9.07 (t, J=6.0 Hz, 1H), 8.55 (d, J=5.7 Hz, 1H), 7.84 (s, 1H), 7.46-7.39 (m, 2H), 7.35 (d, J=2.4 Hz, 1H), 7.32 (dd, J=7.5 Hz, 1.5 Hz, 1H), 7.1-7.09 (m, 1H), 7.09-7.03 (m, 3H), 4.47 (d, J=6.0 Hz, 2H), 2.15 (s, 3H).

ESI-MS: 489.10 (M+H)⁺.

### N-[(3,5-difluorophenyl)methyl]-2-methyl-3-({2-[5-(trifluoromethyl)-1H-imidazol-2-yl]-4-pyridyl}oxy)benzamide (314)

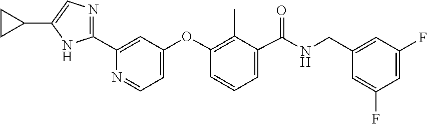 [SMILES: Cc1c(Oc2ccnc(-c3ncc(C4CC4)[nH]3)c2)cccc1C(=O)NCc1cc(F)cc(F)c1]

Compound 314 was synthesized from intermediate 310 (0.07 mmol) and 2-bromo-1-cyclopropylethanone (0.13 mmol) as a white solid in 26% yield according to the general method R. ESI-MS: 461.10 (M+H)⁺.

### Example 15: General Procedure for the Synthesis of Analogues 318

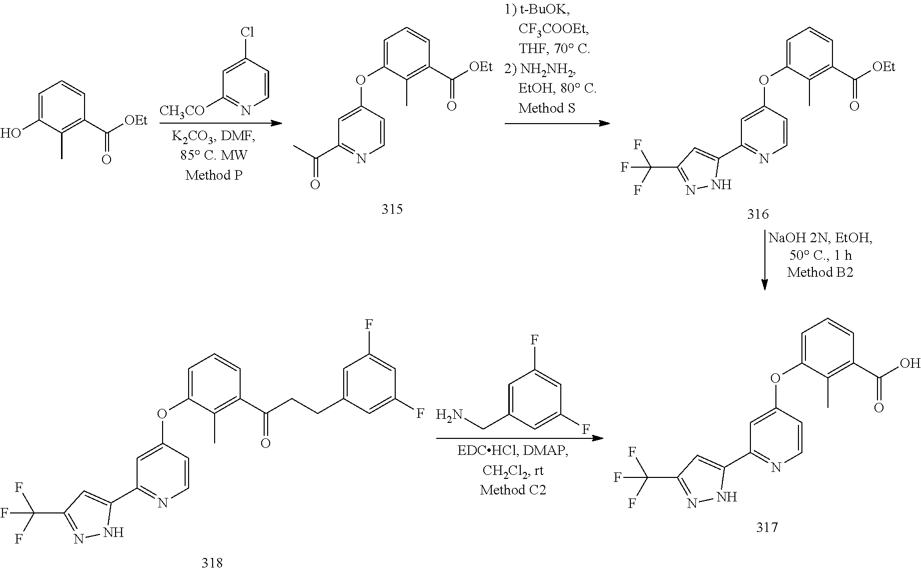

### Preparation of ethyl 3-[(2-acetyl-4-pyridyl)oxy]-2-methyl-benzoate (315)

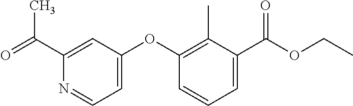 [SMILES: CCOC(=O)c1cccc(Oc2ccnc(C(C)=O)c2)c1C]

Intermediate 315 was synthesized from ethyl 3-hydroxy-2-methyl-benzoate (0.96 mmol) and 1-(4-chloro-2-pyridyl) ethanone (0.64 mmol) as an orange oil in 40% yield according to the general method P.

ESI-MS: 299.95 (M+H)⁺.
Method S: To a stirred solution of 315 (20 mg, 0.07 mmol) in THF (1 mL) was added at 0° C. t-BuOK (1M THF, 0.134 mL, 0.13 mmol). After 5 min, ethyl trifluoroacetate (16 μL, 0.13 mmol) was added and the reaction mixture was stirred at 70° C. for 4 h. After cooling to room temperature, t-BuOK (1M THF, 0.134 mL, 0.13 mmol) and ethyl trifluoroacetate (16 μL, 0.13 mmol) were added and the reaction mixture was stirred at 70° C. overnight. The reaction mixture was diluted with EtOAc and washed twice with a saturated solution of NH₄Cl. The organic layer was dried over Na₂SO₄, filtered and concentrated under reduced pressure. The intermediate was dissolved in EtOH (1 mL) and hydrazine hydrate was added (9 μL, 0.18 mmol). The reaction mixture was stirred at 80° C. overnight. The reaction mixture was concentrated under reduce pressure and ethyl 2-methyl-3-({2-[3-(trifluoromethyl)-1H-pyrazol-5-yl]-4-pyridyl}oxy)benzoate 316 was directly used in the next step without purification.

ESI-MS: 392.00 (M+H)⁺.

### Preparation of 2-methyl-3-({2-[3-(trifluoromethyl)-1H-pyrazol-5-yl]-4-pyridyl}oxy)benzoic acid (317)

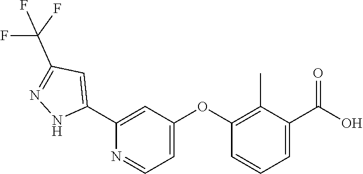 [SMILES: Cc1c(Oc2ccnc(-c3cc(C(F)(F)F)n[nH]3)c2)cccc1C(=O)O]

Intermediate 317 was synthesized from 316 according to the general method B2.

ESI-MS: 363.95 (M+H)⁺.

### N-[(3,5-difluorophenyl)methyl]-2-methyl-3-({2-[3-(trifluoromethyl)-1H-pyrazol-5-yl]-4-pyridyl}oxy)benzamide (318)

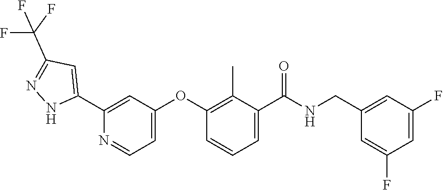 [SMILES: Cc1c(Oc2ccnc(-c3cc(C(F)(F)F)n[nH]3)c2)cccc1C(=O)NCc1cc(F)cc(F)c1]

Compound 318 was synthesized from intermediate 317 and 3,5-difluorobenzylamine as a white solid in 9% yield according to the general method C2. ¹H NMR (600 MHz, DMSO-d₆) δ (ppm): 14.34 (bs, 1H), 9.00 (t, J=5.9 Hz, 1H), 8.50 (d, J=5.7 Hz, 1H), 7.60 (s, 1H), 7.42-7.38 (m, 2H), 7.31 (s, 1H), 7.27 (dd, J=7.5 Hz, 1.8 Hz, 1H), 7.12 (tt, J=9.3 Hz, 2.3 Hz, 1H), 7.09-7.04 (m, 2H), 6.73 (dd, J=5.7 Hz, 2.4 Hz, 1H), 4.48 (d, J=6.0 Hz, 2H), 2.15 (s, 3H).

ESI-MS: 489.10 (M+H)⁺.

### Example 16: General Procedure for the Synthesis of Analogues 321 and 322

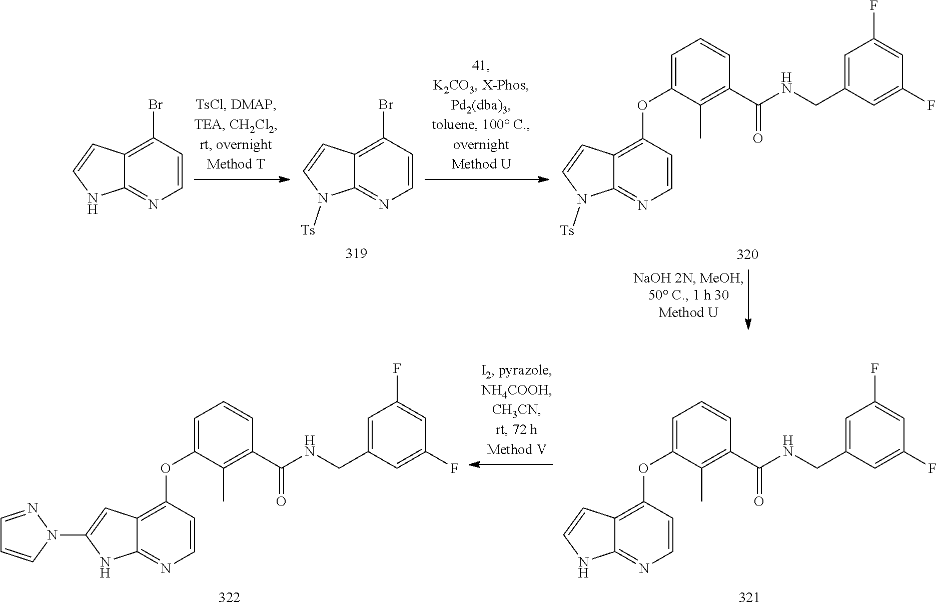 [SMILES: Brc1ccnc2[nH]ccc12 ; Cc1ccc(S(=O)(=O)n2ccc3c(Br)ccnc32)cc1 ; Cc1ccc(S(=O)(=O)n2ccc3c(Oc4cccc(C(=O)NCc5cc(F)cc(F)c5)c4C)ccnc32)cc1 ; Cc1c(Oc2ccnc3[nH]c(-n4cccn4)cc23)cccc1C(=O)NCc1cc(F)cc(F)c1 ; Cc1c(Oc2ccnc3[nH]ccc23)cccc1C(=O)NCc1cc(F)cc(F)c1]

Method T: To a stirred solution of 4-bromo-7-azaindole (2.5 g, 12.69 mmol) in dichloromethane (40 mL) were added DMAP (155 mg, 1.27 mmol), triethylamine (2.1 mL, 15.23 mmol) and tosyl chloride (2.66 g, 13.96 mmol). The reaction mixture was stirred at room temperature overnight. The reaction mixture was concentrated under reduced pressure and the crude was purified by flash column chromatography (Cyclohexane/EtOAc from 100/0 to 60/40) to give 3.914 g of 4-bromo-1-(p-tolylsulfonyl) pyrrolo[2,3-b]pyridine 319 as a yellow powder in 88% yield.

ESI-MS: 350.85-352.80 (M+H)⁺.
Method U: To a stirred solution of intermediate 319 (200 mg, 0.57 mmol) in toluene (10 mL) were added under nitrogen intermediate 41 (237 mg, 0.85 mmol), K₂CO₃ (197 mg, 1.42 mmol), X-Phos (54 mg, 0.11 mmol), and Pd₂(dba)₃ (52 mg, 0.06 mmol). The reaction mixture was stirred at 100° C. overnight. The reaction mixture was diluted with EtOAc and washed twice with a saturated solution of NH₄Cl. The organic layer was dried over Na₂SO₄, filtered and concentrated under reduced pressure. The crude was purified by flash column chromatography (Cyclohexane/EtOAc from 100/0 to 50/50) to give 200 mg of N-[(3,5-difluorophenyl)methyl]-2-methyl-3-[1-(p-tolylsulfonyl) pyrrolo[2,3-b]pyridin-4-yl]oxy-benzamide 320 as a white powder in 93% yield.

ESI-MS: 548.10 (M+H)⁺.

### N-[(3,5-difluorophenyl)methyl]-2-methyl-3-(1H-pyrrolo[2,3-b]pyridin-4-yloxy)benzamide (321)

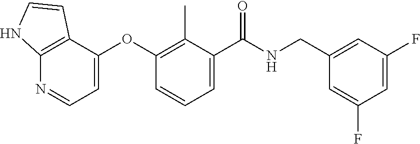 [SMILES: Cc1c(Oc2ccnc3[nH]ccc23)cccc1C(=O)NCc1cc(F)cc(F)c1]

Compound 321 was synthesized from intermediate 320 (0.49 mmol) and NaOH (2.46 mmol) as a white solid in 87% yield according to the general method B2. ¹H NMR (400 MHz, DMSO-d₆) δ (ppm): 11.74 (bs, 1H), 9.02 (t, J=6.0, 1H), 8.06 (d, J=5.4, 1H), 7.39-7.32 (m, 3H), 7.20 (dd, J=6.7, 2.6, 1H), 7.15-7.02 (m, 3H), 6.26 (d, J=5.4, 1H), 6.23 (d, J=3.4, 1H), 4.47 (d, J=6.0, 2H), 2.15 (s, 3H).

ESI-MS: 394.05 (M+H)⁺.
Method V: To a stirred solution of intermediate 321 (30 mg, 0.08 mmol) in acetonitrile (0.15 mL) were added pyrazole (18 mg, 0.27 mmol), 12 (48 mg, 0.19 mmol), and a saturated aqueous solution of ammonium formate (0.15 mL). The reaction mixture was stirred at room temperature for 72 h. The reaction mixture was diluted with EtOAc and washed a saturated solution of Na₂S₂O₃. The organic layer was dried over Na₂SO₄, filtered and concentrated under reduced pressure. The crude was purified by flash column chromatography (DCM/MeOH from 100/0 to 94/6) and reverse phase chromatography (H₂O/MeOH from 100/0 to 0/100) to give N-[(3,5-difluorophenyl)methyl]-2-methyl-3-[(2-pyrazol-1-yl-1H-pyrrolo[2,3-b]pyridin-4-yl)oxy] benzamide 322 as a white powder in 11% yield. ¹H NMR (400 MHz, DMSO-d₆) δ (ppm): 12.68 (bs, 1H), 9.02 (t, J=6.1, 1H), 8.47 (d, J=2.4, 1H), 8.06 (d, J=5.5, 1H), 7.81 (d, J=1.5, 1H), 7.40-7.34 (m, 2H), 7.22 (dd, J=6.9, 2.4, 1H), 7.16-7.05 (m, 3H), 6.59-6.58 (m, 1H), 6.49 (s, 1H), 6.31 (d, J=5.5, 1H), 4.48 (d, J=6.0, 2H), 2.18 (s, 3H).

ESI-MS: 460.00 (M+H)⁺.

### Example 17: General Procedure for the Synthesis of Analogues 327-338

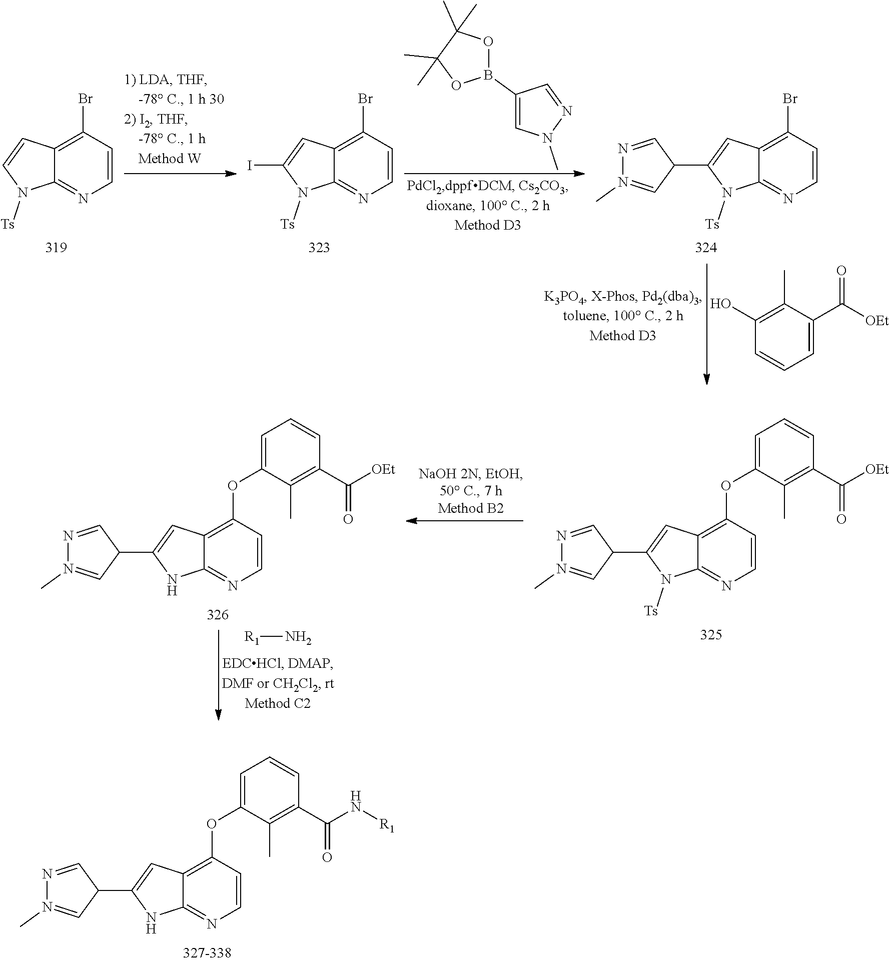

Method W: To a stirred solution of intermediate 319 (2 g, 5.70 mmol) in dry THF (45 mL) under nitrogen was added LDA (1M in hexane, 6.8 mL, 6.83 mmol) at −78° C. The reaction mixture was stirred at −78° C. for 2 h. Then iodine (2.02 g, 7.97 mmol) in THF (10 mL) was added and the reaction mixture was stirred at −78° C. for 1h. Reaction was quenched with saturated aqueous solution of of NH₄Cl and product was extracted with ethyl acetate. Organic layers were washed with saturated solution of Na₂S₂O₃, dried over Na₂SO₄, filtered and concentrated under reduced pressure. The crude was purified by flash column chromatography (Cyclohexane/EtOAc from 100/0 to 85/15) and recrystallized from acetonitrile to give 4-bromo-2-iodo-1H-pyrrolo[2,3-b]pyridine 323 as a white powder in 56% yield.

ESI-MS: 476.85-478.85 (M+H)⁺.

### Preparation of 4-bromo-2-(1-methylpyrazol-4-yl)-1-(p-tolylsulfonyl) pyrrolo[2,3-b]pyridine (324)

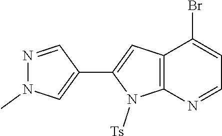 [SMILES: Cc1ccc(S(=O)(=O)n2c(-c3cnn(C)c3)cc3c(Br)ccnc32)cc1]

Intermediate 324 was synthesized from 323 (1.05 mmol), 1-methyl-4-(4,4,5,5-tetramethyl-1,3,2-dioxaborolan-1H-pyrazole (1.05 mmol) as a yellow oil in 55% yield according to the general method D3.

ESI-MS: 430.90-432.90 (M+H)⁺.

### Preparation of ethyl 2-methyl-3-[2-(1-methylpyrazol-4-yl)-1-(p-tolylsulfonyl) pyrrolo[2,3-b]pyridin-4-yl]oxy-benzoate (325)

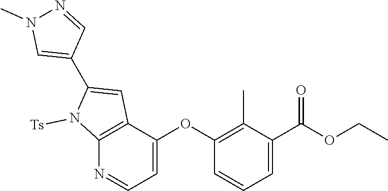 [SMILES: CCOC(=O)c1cccc(Oc2ccnc3c2cc(-c2cnn(C)c2)n3S(=O)(=O)c2ccc(C)cc2)c1C]

Intermediate 325 was synthesized from 324 (0.50 mmol), ethyl 3-hydroxy-2-methyl-benzoate (0.75 mmol) and K₃PO₄ (1.24 mmol) as a yellow oil in 57% yield according to the general method U.

ESI-MS: 531.10 (M+H)⁺.

### Preparation of 2-methyl-3-{[2-(1-methylpyrazol-4-yl)-1H-pyrrolo[2,3-b]pyridin-4-yl]oxy}benzoic acid (326)

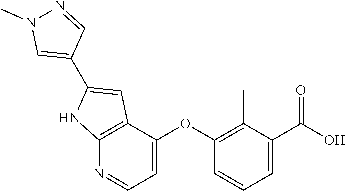 [SMILES: Cc1c(Oc2ccnc3[nH]c(-c4cnn(C)c4)cc23)cccc1C(=O)O]

Intermediate 326 was synthesized from 325 (0.42 mmol) and NaOH 2N (1.27 mmol) as a yellow solid in 92% yield according to the general method B2.

ESI-MS: 349.05 (M+H)⁺.

The following compounds are examples illustrating Method C2:

### N-[(3,5-difluorophenyl)methyl]-2-methyl-3-{[2-(1-methylpyrazol-4-yl)-1H-pyrrolo [2,3-b]pyridin-4-yl]oxy}benzamide (327)

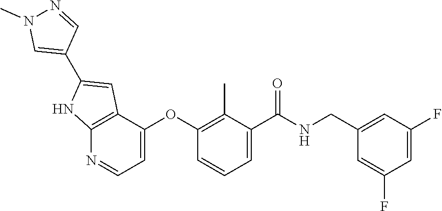 [SMILES: Cc1c(Oc2ccnc3[nH]c(-c4cnn(C)c4)cc23)cccc1C(=O)NCc1cc(F)cc(F)c1]

Compound 327 was synthesized from intermediate 326 (0.05 mmol) and 3,5-difluorobenzylamine (0.08 mmol) as a white solid in 68% yield according to the general method C2. ¹H NMR (400 MHz, DMSO-d₆) δ (ppm): 12.02 (bs, 1H), 9.00 (t, J=5.6, 1H), 8.15 (s, 1H), 7.98 (d, J=5.5, 1H), 7.95 (d, J=0.6, 1H), 7.39-7.33 (m, 2H), 7.20-7.05 (m, 4H), 6.49 (s, 1H), 6.21 (d, J=5.5, 1H), 4.48 (d, J=6.1, 2H), 3.88 (s, 3H), 2.18 (s, 3H).

ESI-MS: 474.10 (M+H)⁺.

### N-[(3-chlorophenyl)methyl]-2-methyl-3-{[2-(1-methylpyrazol-4-yl)-1H-pyrrolo [2,3-b]pyridin-4-yl]oxy}benzamide (328)

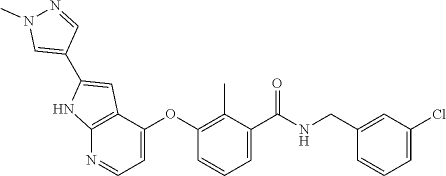 [SMILES: Cc1c(Oc2ccnc3[nH]c(-c4cnn(C)c4)cc23)cccc1C(=O)NCc1cccc(Cl)c1]

Compound 328 was synthesized from intermediate 326 (0.05 mmol) and 3-chlorobenzylamine (0.08 mmol) as a white solid in 29% yield according to the general method C2. ¹H NMR (400 MHz, DMSO-d₆) δ (ppm): 12.03 (bs, 1H), 8.99 (t, J=6.0, 1H), 8.15 (s, 1H), 7.98 (d, J=5.5, 1H), 7.96 (s, 1H), 7.41-7.30 (m, 6H), 7.19 (dd, J=7.7, 1.2, 1H), 6.50 (d, J=2.0, 1H), 6.20 (d, J=5.5, 1H), 4.47 (d, J=6.0, 2H), 3.88 (s, 3H), 2.17 (s, 3H).

ESI-MS: 472.10 (M+H)⁺.

### N-[(4-chlorophenyl)methyl]-2-methyl-3-{[2-(1-methylpyrazol-4-yl)-1H-pyrrolo [2,3-b]pyridin-4-yl]oxy}benzamide (329)

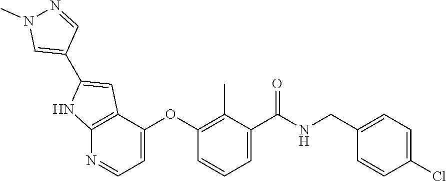 [SMILES: Cc1c(Oc2ccnc3[nH]c(-c4cnn(C)c4)cc23)cccc1C(=O)NCc1ccc(Cl)cc1]

Compound 329 was synthesized from intermediate 326 (0.05 mmol) and 4-chlorobenzylamine (0.08 mmol) as a white solid in 16% yield according to the general method C2. ¹H NMR (400 MHz, DMSO-d₆) δ (ppm): 12.02 (bs, 1H), 8.97 (t, J=6.0, 1H), 8.15 (s, 1H), 7.98 (d, J=5.5, 1H), 7.95 (s, 1H), 7.43-7.30 (m, 6H), 7.18 (dd, J=7.7, 1.3, 1H), 6.49 (d, J=2.0, 1H), 6.19 (d, J=5.5, 1H), 4.44 (d, J=6.0, 2H), 3.88 (s, 3H), 2.16 (s, 3H).

ESI-MS: 472.10 (M+H)⁺.

### N-[(3-fluorophenyl)methyl]-2-methyl-3-{[2-(1-methylpyrazol-4-yl)-1H-pyrrolo [2,3-b]pyridin-4-yl]oxy}benzamide (330)

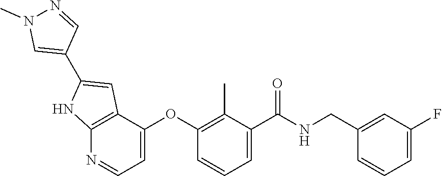 [SMILES: Cc1c(Oc2ccnc3[nH]c(-c4cnn(C)c4)cc23)cccc1C(=O)NCc1cccc(F)c1]

Compound 330 was synthesized from intermediate 326 (0.05 mmol) and 3-fluorobenzylamine (0.08 mmol) as a white solid in 25% yield according to the general method C2. ¹H NMR (400 MHz, DMSO-d₆) δ (ppm): 12.02 (bs, 1H), 8.99 (t, J=6.0, 1H), 8.15 (s, 1H), 7.98 (d, J=5.5, 1H), 7.95 (s, 1H), 7.43-7.31 (m, 3H), 7.21-7.15 (m, 3H), 7.09 (td, J=8.5, 2.2, 1H), 6.49 (d, J=2.0, 1H), 6.20 (d, J=5.5, 1H), 4.48 (d, J=6.0, 2H), 3.88 (s, 3H), 2.17 (s, 3H).

ESI-MS: 456.10 (M+H)⁺.

### N-[(4-fluorophenyl)methyl]-2-methyl-3-{[2-(1-methylpyrazol-4-yl)-1H-pyrrolo [2,3-b]pyridin-4-yl]oxy}benzamide (331)

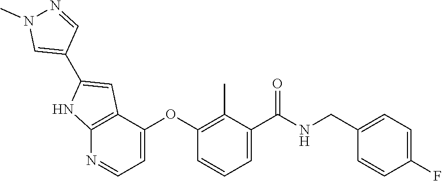 [SMILES: Cc1c(Oc2ccnc3[nH]c(-c4cnn(C)c4)cc23)cccc1C(=O)NCc1ccc(F)cc1]

Compound 331 was synthesized from intermediate 326 (0.05 mmol) and 4-fluorobenzylamine (0.08 mmol) as a white solid in 17% yield according to the general method C2. ¹H NMR (400 MHz, DMSO-d₆) δ (ppm): 12.02 (bs, 1H), 8.95 (t, J=6.0, 1H), 8.15 (s, 1H), 7.98 (d, J=5.5, 1H), 7.95 (s, 1H), 7.42-7.28 (m, 4H), 7.20-7.15 (m, 3H), 6.49 (d, J=1.9, 1H), 6.19 (d, J=5.5, 1H), 4.44 (d, J=6.0, 2H), 3.88 (s, 3H), 2.16 (s, 3H).

ESI-MS: 456.15 (M+H)⁺.

### N-[(6-methoxy-3-pyridyl)methyl]-2-methyl-3-{[2-(1-methylpyrazol-4-yl)-1H-pyrrolo[2,3-b]pyridin-4-yl]oxy}benzamide (332)

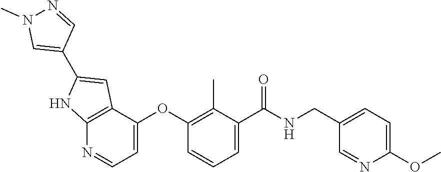

Compound 332 was synthesized from intermediate 326 (0.06 mmol) and (6-methoxypyridin-3-yl) methanamine (0.09 mmol) as a white solid in 55% yield according to the general method C2. ¹H NMR (400 MHz, DMSO-d₆) δ (ppm): 12.02 (bs, 1H), 8.91 (t, J=5.9, 1H), 8.15-8.14 (m, 2H), 7.97 (d, J=5.5, 1H), 7.95 (d, J=0.7, 1H), 7.70 (dd, J=8.5, 2.5, 1H), 7.34 (t, J=7.7, 1H), 7.28 (dd, J=7.6, 1.3, 1H), 7.17 (dd, J=7.9, 1.2, 1H), 6.81 (dd, J=8.5, 0.6, 1H), 6.48 (d, J=2.1, 1H), 6.19 (d, J=5.5, 1H), 4.39 (d, J=5.9, 2H), 3.88 (s, 3H), 3.83 (s, 3H), 2.15 (s, 3H).

ESI-MS: 469.10 (M+H)⁺.

### N-[(5-fluoro-6-methoxy-3-pyridyl)methyl]-2-methyl-3-{[2-(1-methylpyrazol-4-yl)-1H-pyrrolo[2,3-b]pyridin-4-yl]oxy}benzamide (333)

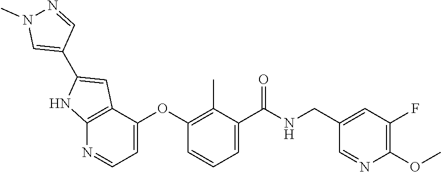

Compound 333 was synthesized from intermediate 326 (0.06 mmol) and (5-fluoro-6-methoxy-3-pyridyl) methanamine (0.09 mmol) as a white solid in 46% yield according to the general method C2. ¹H NMR (400 MHz, DMSO-d₆) δ (ppm): 12.02 (bs, 1H), 8.94 (t, J=5.9, 1H), 8.15 (s, 1H), 7.99-7.95 (m, 3H), 7.65 (dd, J=11.4, 1.9, 1H), 7.36-7.29 (m, 2H), 7.18 (dd, J=7.7, 1.5, 1H), 6.48 (s, 1H), 6.19 (d, J=5.5, 1H), 4.42 (d, J=5.9, 2H), 3.93 (s, 3H), 3.88 (s, 3H), 2.15 (s, 3H).

ESI-MS: 487.15 (M+H)⁺.

### 2-methyl-3-{[2-(1-methylpyrazol-4-yl)-1H-pyrrolo[2,3-b]pyridin-4-yl]oxy}-N-{[6-(trifluoromethyl)-3-pyridyl]methyl} benzamide (334)

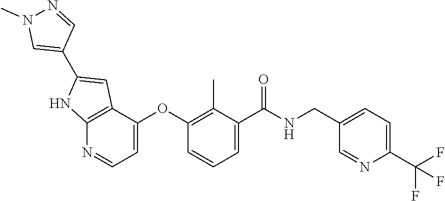 [SMILES: Cc1c(Oc2ccnc3[nH]c(-c4cnn(C)c4)cc23)cccc1C(=O)NCc1ccc(C(F)(F)F)nc1]

Compound 334 was synthesized from intermediate 326 (0.06 mmol) and [6-(trifluoromethyl)-3-pyridyl]methanamine (0.09 mmol) as a white solid in 55% yield according to the general method C2. ¹H NMR (400 MHz, DMSO-d₆) δ (ppm): 12.03 (bs, 1H), 9.09 (t, J=5.9, 1H), 8.77 (d, J=1.6, 1H), 8.15 (s, 1H), 8.05 (dd, J=8.1, 1.5, 1H), 7.98 (d, J=5.5, 1H), 7.95 (d, J=0.7, 1H), 7.91 (d, J=8.1, 1H), 7.38-7.34 (m, 2H), 7.21-7.18 (m, 1H), 6.49 (s, 1H), 6.19 (d, J=5.5, 1H), 4.59 (d, J=5.9, 2H), 3.88 (s, 3H), 2.17 (s, 3H).

ESI-MS: 507.10 (M+H)⁺.

### N-[(3,4-difluorophenyl)methyl]-2-methyl-3-{[2-(1-methylpyrazol-4-yl)-1H-pyrrolo [2,3-b]pyridin-4-yl]oxy}benzamide (335)

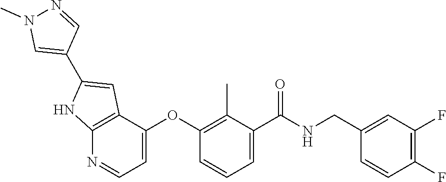 [SMILES: Cc1c(Oc2ccnc3[nH]c(-c4cnn(C)c4)cc23)cccc1C(=O)NCc1ccc(F)c(F)c1]

Compound 335 was synthesized from intermediate 326 (0.06 mmol) and 3,4-difluorobenzylamine (0.09 mmol) as a white solid in 44% yield according to the general method C2. ¹H NMR (400 MHz, DMSO-d₆) δ (ppm): 12.03 (bs, 1H), 8.98 (t, J=6.0, 1H), 8.15 (s, 1H), 7.98 (d, J=5.5, 1H), 7.95 (d, J=0.6, 1H), 7.45-7.31 (m, 4H), 7.23-7.17 (m, 2H), 6.49 (s, 1H), 6.19 (d, J=5.5, 1H), 4.44 (d, J=6.0, 2H), 3.88 (s, 3H), 2.16 (s, 3H).

ESI-MS: 474.05 (M+H)⁺.

### N-[(4-chloro-3-fluoro-phenyl)methyl]-2-methyl-3-{[2-(1-methylpyrazol-4-yl)-1H-pyrrolo[2,3-b]pyridin-4-yl]oxy}benzamide (336)

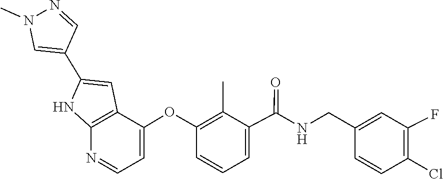 [SMILES: Cc1c(Oc2ccnc3[nH]c(-c4cnn(C)c4)cc23)cccc1C(=O)NCc1ccc(Cl)c(F)c1]

Compound 336 was synthesized from intermediate 326 (0.06 mmol) and 4-chloro-3-fluorobenzylamine (0.09 mmol) as a white solid in 50% yield according to the general method C2. ¹H NMR (400 MHz, DMSO-d₆) δ (ppm): 12.03 (bs, 1H), 9.00 (t, J=6.0, 1H), 8.15 (s, 1H), 7.98 (d, J=5.5, 1H), 7.96 (s, 1H), 7.57 (t, J=8.0, 1H), 7.39-7.32 (m, 3H), 7.24 (dd, J=8.3, 1.2, 1H), 7.19 (dd, J=7.3, 1.9, 1H), 6.49 (s, 1H), 6.20 (d, J=5.5, 1H), 4.46 (d, J=6.0, 2H), 3.88 (s, 3H), 2.17 (s, 3H).

ESI-MS: 490.10 (M+H)⁺.

### 2-methyl-3-{[2-(1-methylpyrazol-4-yl)-1H-pyrrolo[2,3-b]pyridin-4-yl]oxy}-N-(4-pyridylmethyl)benzamide (337)

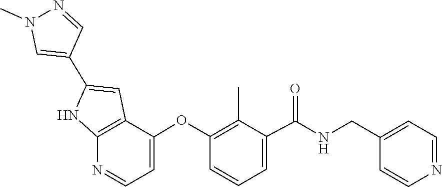 [SMILES: Cc1c(Oc2ccnc3[nH]c(-c4cnn(C)c4)cc23)cccc1C(=O)NCc1ccncc1]

Compound 337 was synthesized from intermediate 326 (0.06 mmol) and 4-(aminomethyl)pyridine (0.08 mmol) as a white solid in 63% yield according to the general method C2. ¹H NMR (400 MHz, DMSO-d₆) δ (ppm): 12.03 (bs, 1H), 9.04 (t, J=6.0, 1H), 8.54-8.52 (m, 2H), 8.15 (s, 1H), 7.98 (d, J=5.5, 1H), 7.96 (s, 1H), 7.38-7.34 (m, 4H), 7.20 (p, J=3.6, 1H), 6.50 (s, 1H), 6.20 (d, J=5.5, 1H), 4.49 (d, J=6.0, 2H), 3.88 (s, 3H), 2.19 (s, 3H).

ESI-MS: 439.15 (M+H)⁺.

### 2-methyl-3-{[2-(1-methylpyrazol-4-yl)-1H-pyrrolo[2,3-b]pyridin-4-yl]oxy}-N-(3-pyridylmethyl)benzamide (338)

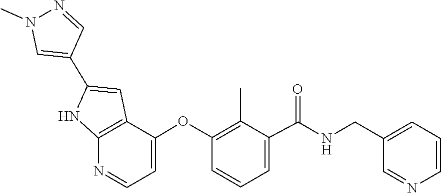 [SMILES: Cc1c(Oc2ccnc3[nH]c(-c4cnn(C)c4)cc23)cccc1C(=O)NCc1cccnc1]

Compound 338 was synthesized from intermediate 326 (0.06 mmol) and 3-(aminomethyl)pyridine (0.08 mmol) as a white solid in 70% yield according to the general method C2. ¹H NMR (400 MHz, DMSO-d₆) δ (ppm): 12.02 (bs, 1H), 9.00 (t, J=6.0, 1H), 8.58 (d, J=1.8, 1H), 8.48 (dd, J=4.8, 1.6, 1H), 8.15 (s, 1H), 7.98 (d, J=5.5, 1H), 7.95 (d, J=0.5, 1H), 7.77 (dt, J=7.8, 1.9, 1H), 7.40-7.30 (m, 3H), 7.18 (dd, J=7.6, 1.6, 1H), 6.49 (s, 1H), 6.19 (d, J=5.5, 1H), 4.49 (d, J=5.9, 2H), 3.88 (s, 3H), 2.16 (s, 3H). 10 ESI-MS: 439.10 (M+H)⁺.

### Example 18: General Procedure for the Synthesis of Analogues 341-344

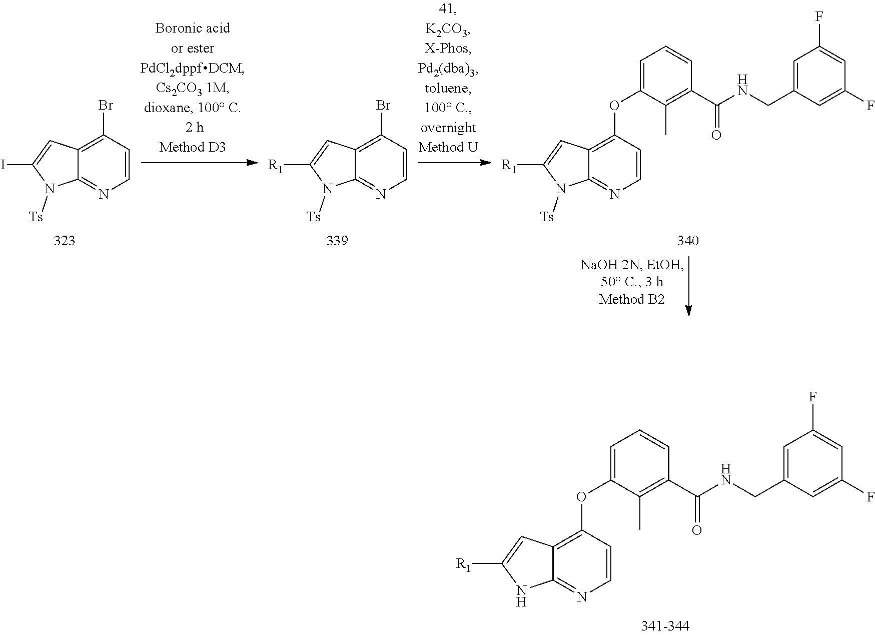

The following table illustrates intermediates 339 prepared from method D3:

| Intermediate | Structure | Synthesis procedure |
|---|---|---|
| Compound 339a | 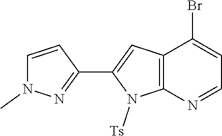 [SMILES: Cc1ccc(S(=O)(=O)n2c(-c3ccn(C)n3)cc3c(Br)ccnc32)cc1] | Method D3 |
| Compound 339b | 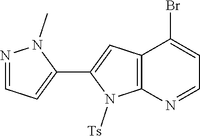 [SMILES: Cc1ccc(S(=O)(=O)n2c(-c3ccnn3C)cc3c(Br)ccnc32)cc1] | Method D3 |
| Compound 339c | 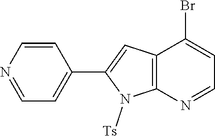 [SMILES: Cc1ccc(S(=O)(=O)n2c(-c3ccncc3)cc3c(Br)ccnc32)cc1] | Method D3 |
| Compound 339d | 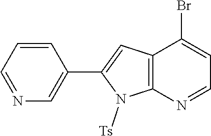 [SMILES: Cc1ccc(S(=O)(=O)n2c(-c3cccnc3)cc3c(Br)ccnc32)cc1] | Method D3 |

The following table illustrates intermediates 340 prepared from method U:

| | | Synthesis |
|---|---|---|
| Intermediate | Structure | procedure |
| Compound 340a | 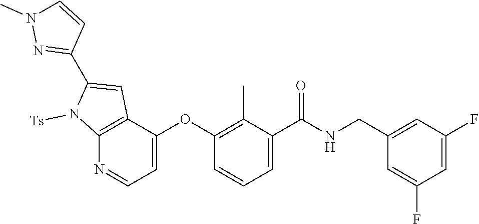 [SMILES: Cc1ccc(S(=O)(=O)n2c(-c3ccn(C)n3)cc3c(Oc4cccc(C(=O)NCc5cc(F)cc(F)c5)c4C)ccnc32)cc1] | Method U |
| Compound 340b | 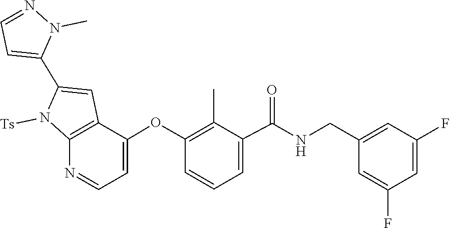 [SMILES: Cc1ccc(S(=O)(=O)n2c(-c3ccnn3C)cc3c(Oc4cccc(C(=O)NCc5cc(F)cc(F)c5)c4C)ccnc32)cc1] | Method U |

### N-[(3,5-difluorophenyl)methyl]-2-methyl-3-{[2-(4-pyridyl)-1H-pyrrolo[2,3-b] pyridin-4-yl]oxy}benzamide (341)

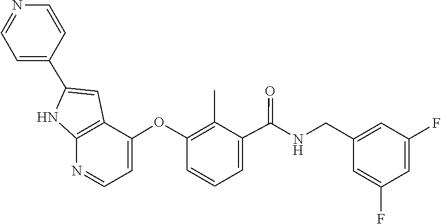 [SMILES: Cc1c(Oc2ccnc3[nH]c(-c4ccncc4)cc23)cccc1C(=O)NCc1cc(F)cc(F)c1]

Compound 341 was synthesized from intermediate 339c (0.13 mmol) and 41 (0.18 mmol) as a white solid in 10% yield according to the general method U. 1H NMR (400 MHz, DMSO-d₆) δ (ppm): 12.55 (bs, 1H), 9.03 (t, J=6.1, 1H), 8.63-8.61 (m, 2H), 8.14 (d, J=5.5, 1H), 7.93-7.91 (m, 2H), 7.43-7.37 (m, 2H), 7.31-7.26 (m, 1H), 7.24 (s, 1H), 7.16-7.04 (m, 3H), 6.21 (d, J=5.5, 1H), 4.49 (d, J=6.0, 2H), 2.17 (s, 3H).

ESI-MS: 471.05 (M+H)⁺.

### N-[(3,5-difluorophenyl)methyl]-2-methyl-3-{[2-(3-pyridyl)-1H-pyrrolo[2,3-b] pyridin-4-yl]oxy}benzamide (342)

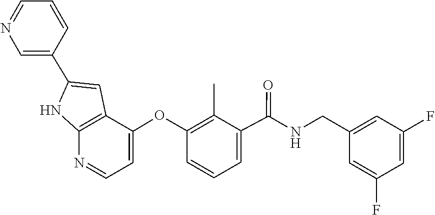 [SMILES: Cc1c(Oc2ccnc3[nH]c(-c4cccnc4)cc23)cccc1C(=O)NCc1cc(F)cc(F)c1]

Compound 342 was synthesized from intermediate 339d (0.12 mmol) and 41 (0.17 mmol) as a white solid in 4% yield according to the general method U. 1H NMR (400 MHz, DMSO-d₆) δ (ppm): 12.43 (bs, 1H), 9.16 (d, J=1.7, 1H), 9.03 (t, J=6.0, 1H), 8.53 (dd, J=4.8, 1.5, 1H), 8.32-8.28 (m, 1H), 8.10 (d, J=5.5, 1H), 7.48 (ddd, J=8.0, 4.8, 0.7, 1H), 7.42-7.37 (m, 2H), 7.26 (dd, J=6.9, 2.4, 1H), 7.16-7.05 (m, 4H), 6.21 (d, J=5.5, 1H), 4.49 (d, J=6.0, 2H), 2.17 (s, 3H).

ESI-MS: 471.05 (M+H)⁺.

The following compounds are examples illustrating Method B2:

### N-[(3,5-difluorophenyl)methyl]-2-methyl-3-{[2-(1-methylpyrazol-3-yl)-1H-pyrrolo [2,3-b]pyridin-4-yl]oxy}benzamide (343)

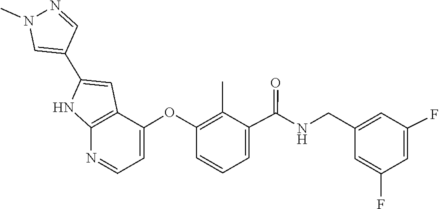 [SMILES: Cc1c(Oc2ccnc3[nH]c(-c4cnn(C)c4)cc23)cccc1C(=O)NCc1cc(F)cc(F)c1]

Compound 343 was synthesized from intermediate 340a (0.11 mmol) and NaOH (0.32 mmol) as a white solid in 60% yield according to the general method B2. ¹H NMR (400 MHz, DMSO-d₆) δ (ppm): 12.12 (bs, 1H), 9.02 (t, J=6.0, 1H), 8.04 (d, J=5.4, 1H), 7.76 (d, J=2.2, 1H), 7.39-7.33 (m, 2H), 7.20 (dd, J=6.9, 2.4, 1H), 7.16-7.05 (m, 3H), 6.79 (d, J=2.3, 1H), 6.55 (d, J=2.1, 1H), 6.28 (d, J=5.4, 1H), 4.48 (d, J=6.0, 2H), 3.89 (s, 3H), 2.19 (s, 3H).

ESI-MS: 474.15 (M+H)⁺.

### N-[(3,5-difluorophenyl)methyl]-2-methyl-3-{[2-(2-methylpyrazol-3-yl)-1H-pyrrolo [2,3-b]pyridin-4-yl]oxy}benzamide (344)

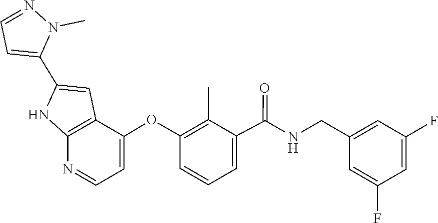 [SMILES: Cc1c(Oc2ccnc3[nH]c(-c4ccnn4C)cc23)cccc1C(=O)NCc1cc(F)cc(F)c1]

Compound 344 was synthesized from intermediate 340b (0.09 mmol) and NaOH (0.46 mmol) as a white solid in 18% yield according to the general method B2. ¹H NMR (400 MHz, DMSO-d₆) δ (ppm): 12.24 (bs, 1H), 9.03 (t, J=6.0, 1H), 8.12 (d, J=5.5, 1H), 7.49 (d, J=1.9, 1H), 7.42-7.37 (m, 2H), 7.28 (dd, J=6.5, 2.8, 1H), 7.16-7.04 (m, 3H), 6.78 (d, J=1.9, 1H), 6.61 (d, J=2.0, 1H), 6.26 (d, J=5.5, 1H), 4.48 (d, J=6.0, 2H), 4.01 (s, 3H), 2.17 (s, 3H).

ESI-MS: 474.15 (M+H)⁺.

### Example 19: General Procedure for the Synthesis of Analogues 347-349

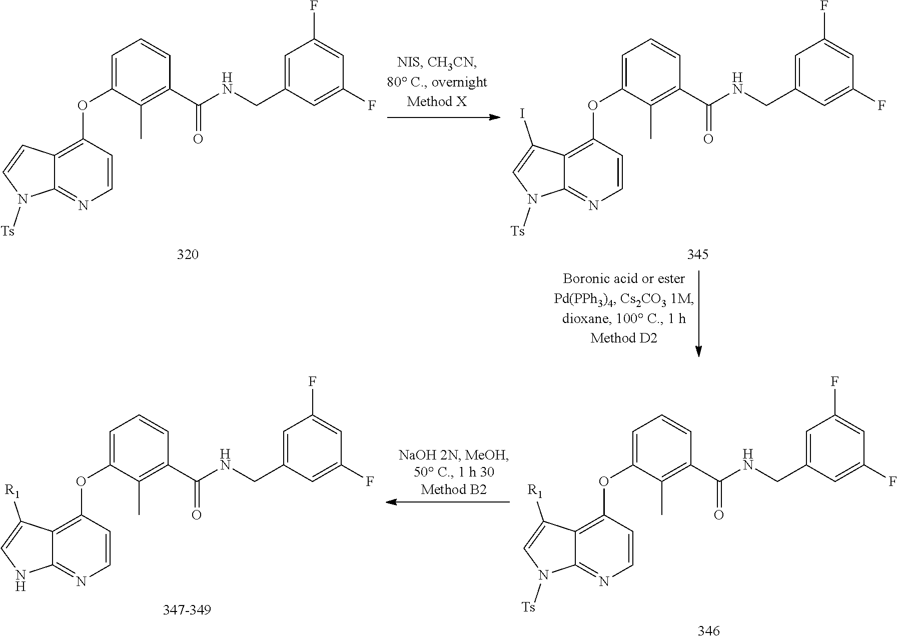

Method X: To a stirred solution of intermediate 320 (100 mg, 0.18 mmol) in acetonitrile (2 mL) was added NIS (82 mg, 0.37 mmol). The reaction mixture was stirred at 80° C. overnight. The reaction mixture was diluted with EtOAc and washed a saturated solution of Na₂S₂O₃. The organic layer was dried over Na₂SO₄, filtered and concentrated under reduced pressure. The crude was purified by flash column chromatography (DCM/EtOAc from 100/0 to 90/10) to give N-[(3,5-difluorophenyl)methyl]-3-[3-iodo-1-(p-tolylsulfonyl) pyrrolo[2,3-b]pyridin-4-yl]oxy-2-methyl-benzamide 345 as a beige powder in 39% yield.

The following table illustrates intermediates 346 prepared from method D2:

| | | Synthesis |
|---|---|---|
| Intermediate | Structure | procedure |
| Compound 346a | 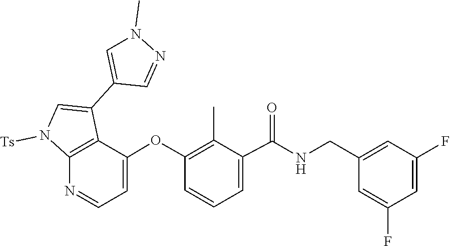 [SMILES: Cc1ccc(S(=O)(=O)n2cc(-c3cnn(C)c3)c3c(Oc4cccc(C(=O)NCc5cc(F)cc(F)c5)c4C)ccnc32)cc1] | Method D2 |
| Compound 346b | 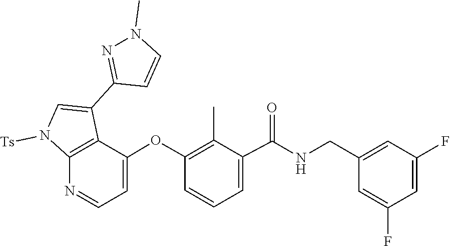 [SMILES: Cc1ccc(S(=O)(=O)n2cc(-c3ccn(C)n3)c3c(Oc4cccc(C(=O)NCc5cc(F)cc(F)c5)c4C)ccnc32)cc1] | Method D2 |
| Compound 346c | 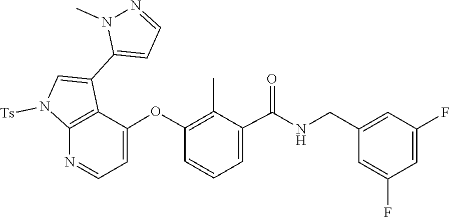 [SMILES: Cc1ccc(S(=O)(=O)n2cc(-c3ccnn3C)c3c(Oc4cccc(C(=O)NCc5cc(F)cc(F)c5)c4C)ccnc32)cc1] | Method D2 |

The following compounds are examples illustrating Method B2:

### N-[(3,5-difluorophenyl)methyl]-2-methyl-3-{[3-(1-methylpyrazol-4-yl)-1H-pyrrolo [2,3-b]pyridin-4-yl]oxy}benzamide (347)

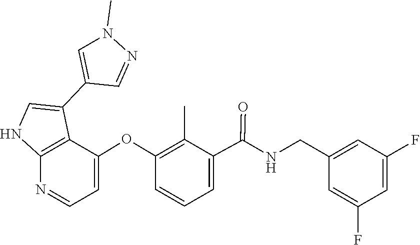 [SMILES: Cc1c(Oc2ccnc3[nH]cc(-c4cnn(C)c4)c23)cccc1C(=O)NCc1cc(F)cc(F)c1]

Compound 347 was synthesized from intermediate 346a (0.02 mmol) and NaOH (0.11 mmol) as a white solid in 90% yield according to the general method B2.

¹H NMR (400 MHz, DMSO-d₆) δ (ppm): 11.82 (bs, 1H), 9.01 (t, J=6.0, 1H), 8.02 (d, J=5.4, 1H), 7.86 (s, 1H), 7.73 (d, J=0.5, 1H), 7.56 (d, J=1.7, 1H), 7.40-7.33 (m, 2H), 7.24 (dd, J=7.5, 1.8, 1H), 7.15-7.04 (m, 3H), 6.08 (d, J=5.4, 1H), 4.48 (d, J=6.0, 2H), 3.80 (s, 3H), 2.14 (s, 3H).

ESI-MS: 474.00 (M+H)⁺.

### N-[(3,5-difluorophenyl)methyl]-2-methyl-3-{[3-(1-methylpyrazol-3-yl)-1H-pyrrolo [2,3-b]pyridin-4-yl]oxy}benzamide (348)

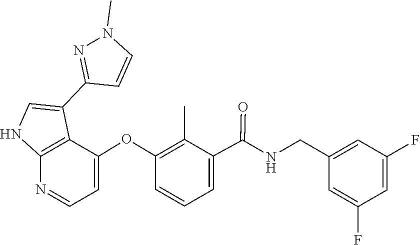 [SMILES: Cc1c(Oc2ccnc3[nH]cc(-c4ccn(C)n4)c23)cccc1C(=O)NCc1cc(F)cc(F)c1]

Compound 348 was synthesized from intermediate 346b (0.02 mmol) and NaOH (0.12 mmol) as a white solid in 72% yield according to the general method B2. ¹H NMR (400 MHz, DMSO-d₆) δ (ppm): 11.91 (bs, 1H), 9.00 (t, J=6.1, 1H), 8.05 (d, J=5.4, 1H), 7.63 (d, J=2.2, 1H), 7.59 (d, J=2.1, 1H), 7.39-7.31 (m, 2H), 7.21 (dd, J=7.6, 1.7, 1H), 7.15-7.03 (m, 3H), 6.59 (d, J=2.2, 1H), 6.13 (d, J=5.4, 1H), 4.47 (d, J=6.0, 2H), 3.82 (s, 3H), 2.15 (s, 3H).

ESI-MS: 474.00 (M+H)⁺.

### N-[(3,5-difluorophenyl)methyl]-2-methyl-3-{[3-(2-methylpyrazol-3-yl)-1H-pyrrolo [2,3-b]pyridin-4-yl]oxy}benzamide (349)

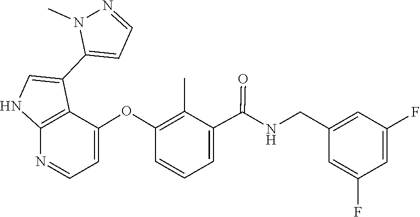 [SMILES: Cc1c(Oc2ccnc3[nH]cc(-c4ccnn4C)c23)cccc1C(=O)NCc1cc(F)cc(F)c1]

Compound 349 was synthesized from intermediate 346c (0.02 mmol) and NaOH (0.08 mmol) as a white solid in 50% yield according to the general method B2. ¹H NMR (400 MHz, DMSO-d₆) δ (ppm): 12.20 (bs, 1H), 8.95 (t, J=6.1, 1H), 8.11 (d, J=5.5, 1H), 7.62 (d, J=2.3, 1H), 7.38 (d, J=1.8, 1H), 7.36-7.28 (m, 2H), 7.18 (dd, J=7.7, 1.5, 1H), 7.15-7.02 (m, 3H), 6.32 (d, J=1.8, 1H), 6.17 (d, J=5.5, 1H), 4.45 (d, J=6.0, 2H), 3.78 (s, 3H), 2.01 (s, 3H). ESI-MS: 473.95 (M+H)⁺.

### Example 20: General Procedure for the Synthesis of Analogue 356

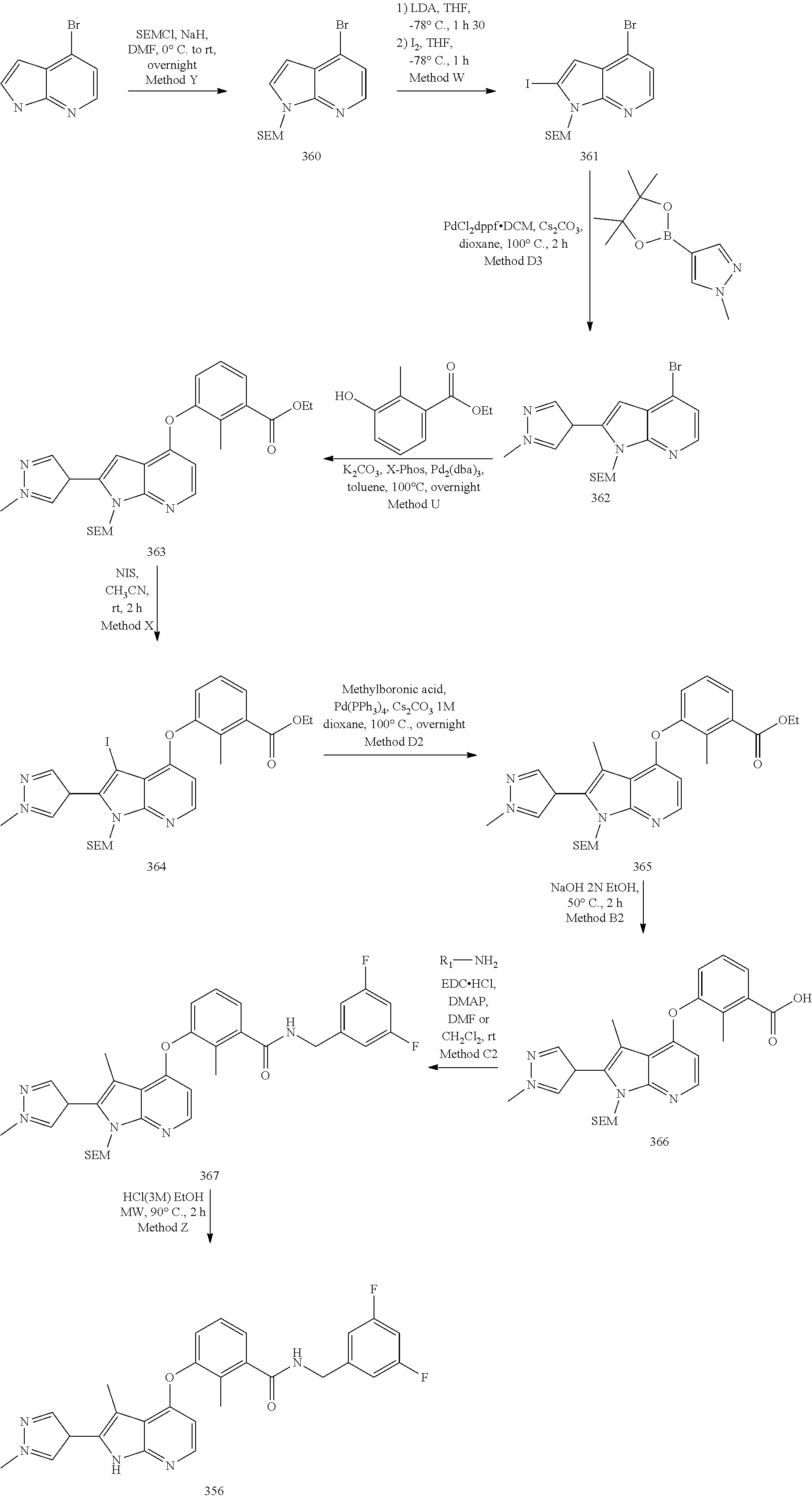

Method Y: To a stirred solution of 4-bromo-7-azaindole (500 mg, 2.54 mmol) in DMF (6 mL) was added under nitrogen at 0° C. NaH (60%) (155 mg, 3.88 mmol). The reaction mixture was stirred at room temperature for 30 minutes and SEM-CI was added (0.538 mL, 3.07 mmol). The reaction mixture was stirred at room temperature overnight. The reaction mixture was diluted with EtOAc and washed with H₂O and brine. The organic layer was dried over Na₂SO₄, filtered and concentrated under reduced pressure. The crude was purified by flash column chromatography (Cyclohexane/EtOAc from 100/0 to 90/10) to give 683 mg of 2-[(4-bromopyrrolo[2,3-b]pyridin-1-yl) methoxy]ethyl-trimethylsilane 360 as a colorless oil in 82% yield.

ESI-MS: 326.85-328.85 (M+H)⁺.

### Preparation of 2-[(4-bromo-2-iodo-pyrrolo[2,3-b]pyridin-1-yl)methoxy]ethyl-trimethylsilane (361)

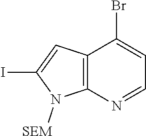 [SMILES: C[Si](C)(C)CCOCn1c(I)cc2c(Br)ccnc21]

Intermediate 361 was synthesized from 360 (3.94 mmol) as a colorless oil in 73% yield according to the general method W.

ESI-MS: 452.80-454.80 (M+H)⁺.

### Preparation of 2-{[4-bromo-2-(1-methylpyrazol-4-yl) pyrrolo[2,3-b]pyridin-1-yl]methoxy}ethyl-trimethylsilane (362)

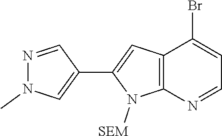 [SMILES: Cn1cc(-c2cc3c(Br)ccnc3n2COCC[Si](C)(C)C)cn1]

Intermediate 362 was synthesized from 361 (4.23 mmol), 1-methyl-4-(4,4,5,5-tetramethyl-1,3,2-dioxaborolan-1H-pyrazole (4.23 mmol) as a yellow oil in 59% yield according to the general method D3.

ESI-MS: 406.95-408.95 (M+H)⁺.

### Preparation of ethyl 2-methyl-3-[2-(1-methylpyrazol-4-yl)-1-(2-trimethylsilyl ethoxymethyl)pyrrolo[2,3-b]pyridin-4-yl]oxy-benzoate (363)

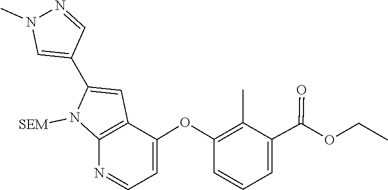 [SMILES: CCOC(=O)c1cccc(Oc2ccnc3c2cc(-c2cnn(C)c2)n3COCC[Si](C)(C)C)c1C]

Intermediate 363 was synthesized from 362 (1.60 mmol), ethyl 3-hydroxy-2-methyl-benzoate (2.40 mmol) and K₂CO₃ (4.01 mmol) as a yellow oil in 99% yield according to the general method U.

ESI-MS: 507.15 (M+H)⁺.

### Preparation of ethyl 3-[3-iodo-2-(1-methylpyrazol-4-yl)-1-(2-trimethylsilylethoxy methyl) pyrrolo[2,3-b]pyridin-4-yl]oxy-2-methyl-benzoate (364)

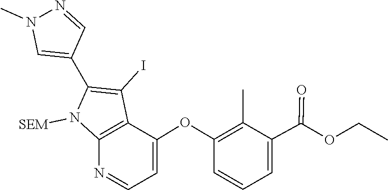 [SMILES: CCOC(=O)c1cccc(Oc2ccnc3c2c(I)c(-c2cnn(C)c2)n3COCC[Si](C)(C)C)c1C]

Intermediate 364 was synthesized from 363 (0.49 mmol) and NIS (0.99 mmol) as a white gum in 82% yield according to the general method X.

ESI-MS: 633.00 (M+H)⁺.

### Preparation of ethyl 2-methyl-3-[3-methyl-2-(1-methylpyrazol-4-yl)-1-(2-trimethylsilylethoxymethyl)pyrrolo[2,3-b]pyridin-4-yl]oxy-benzoate (365)

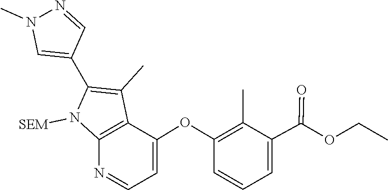 [SMILES: CCOC(=O)c1cccc(Oc2ccnc3c2c(C)c(-c2cnn(C)c2)n3COCC[Si](C)(C)C)c1C]

Intermediate 365 was synthesized from 364 (0.08 mmol) and methylboronic acid (0.80 mmol) as a yellow oil in 90% yield according to the general method D2.

ESI-MS: 521.15 (M+H)⁺.

### Preparation of 2-methyl-3-[3-methyl-2-(1-methylpyrazol-4-yl)-1-(2-trimethylsilyl ethoxymethyl)pyrrolo[2,3-b]pyridin-4-yl]oxy-benzoic acid (366)

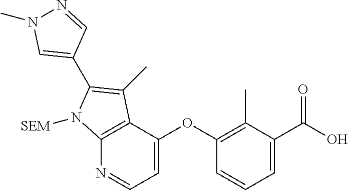 [SMILES: Cc1c(Oc2ccnc3c2c(C)c(-c2cnn(C)c2)n3COCC[Si](C)(C)C)cccc1C(=O)O]

Intermediate 366 was synthesized from 365 (0.14 mmol) and 2N sodium hydroxide (0.43 mmol) as a yellow oil in 100% yield according to the general method B2.

ESI-MS: 493.10 (M+H)⁺.

### Preparation of N-[(3,5-difluorophenyl)methyl]-2-methyl-3-[3-methyl-2-(1-methyl pyrazol-4-yl)-1-(2-trimethylsilylethoxymethyl)pyrrolo[2,3-b]pyridin-4-yl]oxy-benzamide (367)

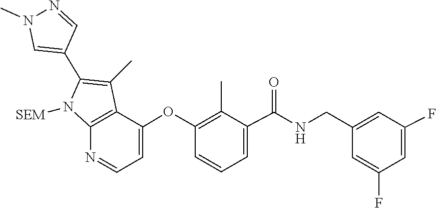 [SMILES: Cc1c(Oc2ccnc3c2c(C)c(-c2cnn(C)c2)n3COCC[Si](C)(C)C)cccc1C(=O)NCc1cc(F)cc(F)c1]

Intermediate 367 was synthesized from 366 (0.14 mmol) and 3,5-difluorobenzylamine (0.22 mmol) as a white solid in 67% yield according to the general method C2.

ESI-MS: 618.20 (M+H)⁺.
Method Z: Intermediate 367 (60 mg, 0.10 mmol) was dissolved in ethanol (1 mL) in an oven-dried screw-cap test tube. HCl 3M (1.2 mL, 3.59f mmol) was added. The reaction mixture was stirred and heated under microwave irradiation at 90° C. for 2 h. The reaction mixture was diluted with EtOAc and washed with a saturated solution of NaHCO₃. The organic layer was dried over Na₂SO₄, filtered and concentrated under reduced pressure. The crude was purified by flash column chromatography (DCM/MeOH from 100/0 to 95/5) and reverse phase chromatography (H₂O/MeOH from 100/0 to 0/100) to give N-[(3,5-difluorophenyl)methyl]-2-methyl-3-{[3-methyl-2-(1-methylpyrazol-4-yl)-1H-pyrrolo[2,3-b]pyridin-4-yl]oxy}benzamide 356 as a white powder in 51% yield. ¹H NMR (400 MHz, DMSO-d₆) δ (ppm): 11.71 (bs, 1H), 9.00 (t, J=6.0, 1H), 8.14 (s, 1H), 7.95-7.91 (m, 2H), 7.42-7.29 (m, 2H), 7.23-7.02 (m, 4H), 6.09 (d, J=5.5, 1H), 4.48 (d, J=6.0, 2H), 3.92 (s, 3H), 2.52 (s, 3H), 2.18 (s, 3H).

ESI-MS: 488.10 (M+H)⁺.

### Example 21: General Procedure for the Synthesis of Analogues 357-358

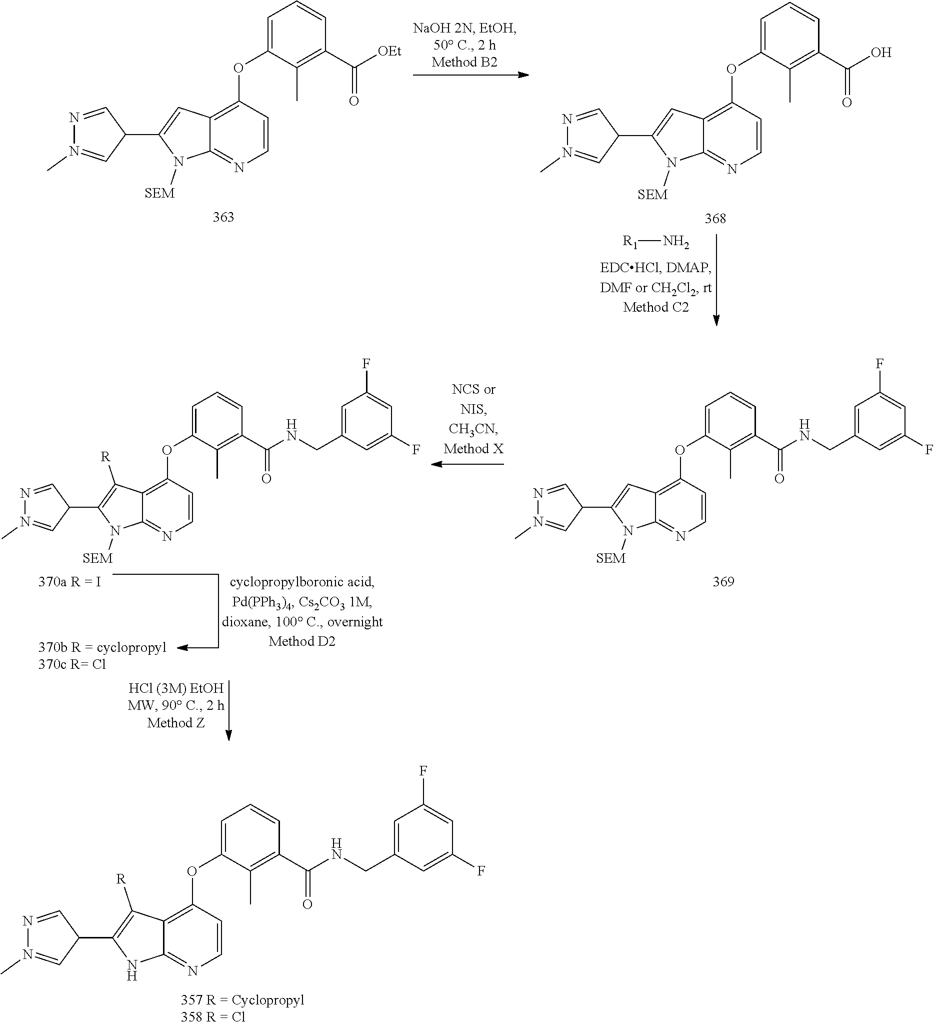

### Preparation of 2-methyl-3-[2-(1-methylpyrazol-4-yl)-1-(2-trimethylsilylethoxy methyl) pyrrolo[2,3-b]pyridin-4-yl]oxy-benzoic acid (368)

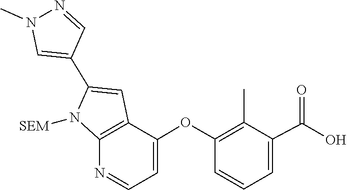 [SMILES: Cc1c(Oc2ccnc3c2cc(-c2cnn(C)c2)n3COCC[Si](C)(C)C)cccc1C(=O)O]

Intermediate 368 was synthesized from 363 (0.72 mmol) and 2N sodium hydroxide (2.16 mmol) as a yellow solid in 83% yield according to the general method B2.

ESI-MS: 479.10 (M+H)⁺.

### Preparation of N-[(3,5-difluorophenyl)methyl]-2-methyl-3-[2-(1-methylpyrazol-4-yl)-1-(2-trimethylsilylethoxymethyl)pyrrolo[2,3-b]pyridin-4-yl]oxy-benzamide (369)

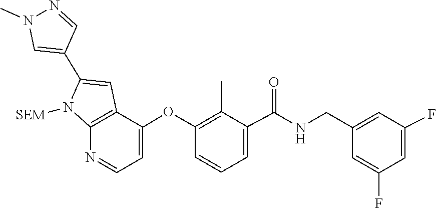 [SMILES: Cc1c(Oc2ccnc3c2cc(-c2cnn(C)c2)n3COCC[Si](C)(C)C)cccc1C(=O)NCc1cc(F)cc(F)c1]

Intermediate 369 was synthesized from 368 (0.60 mmol) and 3,5-difluorobenzylamine (0.89 mmol) as a yellow solid in 78% yield according to the general method C2.

ESI-MS: 604.20 (M+H)⁺.

### Preparation of N-[(3,5-difluorophenyl)methyl]-3-[3-iodo-2-(1-methylpyrazol-4-yl)-1-(2-trimethylsilylethoxymethyl)pyrrolo[2,3-b]pyridin-4-yl]oxy-2-methyl-benzamide (370a)

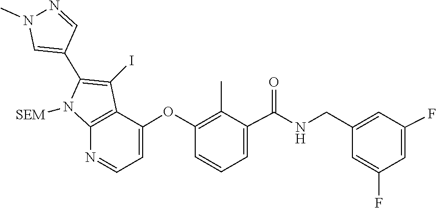 [SMILES: Cc1c(Oc2ccnc3c2c(I)c(-c2cnn(C)c2)n3COCC[Si](C)(C)C)cccc1C(=O)NCc1cc(F)cc(F)c1]

Intermediate 370a was synthesized from 369 (0.08 mmol) and NIS (0.16 mmol) as a yellow oil in 93% yield according to the general method X.

ESI-MS: 730.05 (M+H)⁺.

### Preparation of 3-[3-cyclopropyl-2-(1-methylpyrazol-4-yl)-1-(2-trimethylsilyl ethoxymethyl)pyrrolo[2,3-b]pyridin-4-yl]oxy-N-[(3,5-difluorophenyl)methyl]-2-methyl-benzamide (370b)

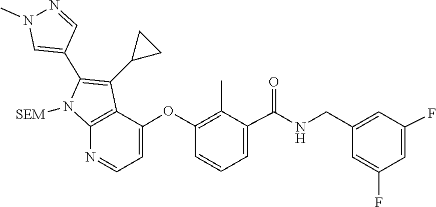 [SMILES: Cc1c(Oc2ccnc3c2c(C2CC2)c(-c2cnn(C)c2)n3COCC[Si](C)(C)C)cccc1C(=O)NCc1cc(F)cc(F)c1]

Intermediate 370b was synthesized from 370a (0.08 mmol) and cyclopropylboronic acid (0.15 mmol) as a colorless oil in 63% yield according to the general method D2.

ESI-MS: 644.20 (M+H)⁺.

### Preparation of 3-[3-chloro-2-(1-methylpyrazol-4-yl)-1-(2-trimethylsilylethoxy methyl) pyrrolo[2,3-b]pyridin-4-yl]oxy-N-[(3,5-difluorophenyl)methyl]-2-methyl-benzamide (370c)

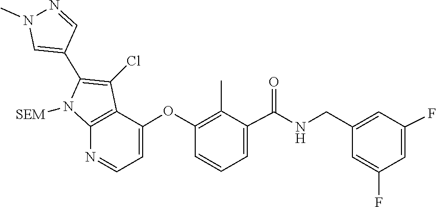 [SMILES: Cc1c(Oc2ccnc3c2c(Cl)c(-c2cnn(C)c2)n3COCC[Si](C)(C)C)cccc1C(=O)NCc1cc(F)cc(F)c1]

Intermediate 370c was synthesized from 369 (0.08 mmol) and NCS (0.16 mmol) as a yellow oil in 100% yield according to the general method X.

ESI-MS: 638.10 (M+H)⁺.

### 3-{[3-cyclopropyl-2-(1-methylpyrazol-4-yl)-1H-pyrrolo[2,3-b]pyridin-4-yl]oxy}-N-[(3,5-difluorophenyl)methyl]-2-methyl-benzamide (357)

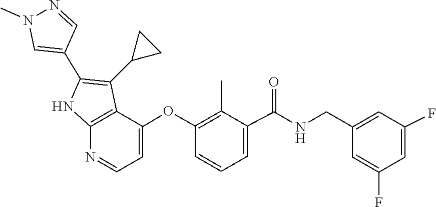 [SMILES: Cc1c(Oc2ccnc3[nH]c(-c4cnn(C)c4)c(C4CC4)c23)cccc1C(=O)NCc1cc(F)cc(F)c1]

Compound 357 was synthesized from intermediate 370b (0.05 mmol) and HCl 3M (1.40 mmol) as a white solid in 29% yield according to the general method Z. ¹H NMR (400 MHz, DMSO-d₆) δ (ppm): 11.72 (bs, 1H), 9.00 (t, J=6.1, 1H), 8.18 (s, 1H), 8.00 (d, J=0.6, 1H), 7.94 (d, J=5.5, 1H), 7.40-7.27 (m, 2H), 7.19-7.02 (m, 4H), 6.10 (d, J=5.5, 1H), 4.49 (d, J=6.1, 2H), 3.93 (s, 3H), 2.23 (s, 3H), 1.92-1.85 (m, 1H), 0.97-0.91 (m, 2H), 0.56-0.52 (m, 2H).

ESI-MS: 514.10 (M+H)⁺.

### 3-{[3-chloro-2-(1-methylpyrazol-4-yl)-1H-pyrrolo[2,3-b]pyridin-4-yl]oxy}-N-[(3,5-difluorophenyl)methyl]-2-methyl-benzamide (358)

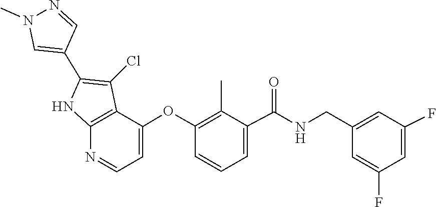 [SMILES: Cc1c(Oc2ccnc3[nH]c(-c4cnn(C)c4)c(Cl)c23)cccc1C(=O)NCc1cc(F)cc(F)c1]

Compound 358 was synthesized from intermediate 370c (0.08 mmol) and HCl 3M (1.25 mmol) as a white solid in 43% yield according to the general method Z. ¹H NMR (400 MHz, DMSO-d₆) δ (ppm): 12.34 (bs, 1H), 9.00 (t, J=6.0, 1H), 8.37 (s, 1H), 8.11 (d, J=0.6, 1H), 8.05 (d, J=5.5, 1H), 7.41-7.32 (m, 2H), 7.23 (dd, J=7.7, 1.6, 1H), 7.16-7.05 (m, 3H), 6.21 (d, J=5.5, 1H), 4.48 (d, J=6.0, 2H), 3.94 (s, 3H), 2.19 (s, 3H).

ESI-MS: 508.05 (M+H)⁺.

### Example 22: General Procedure for the Synthesis of Analogues CC11, CC20, CC24, and CC25

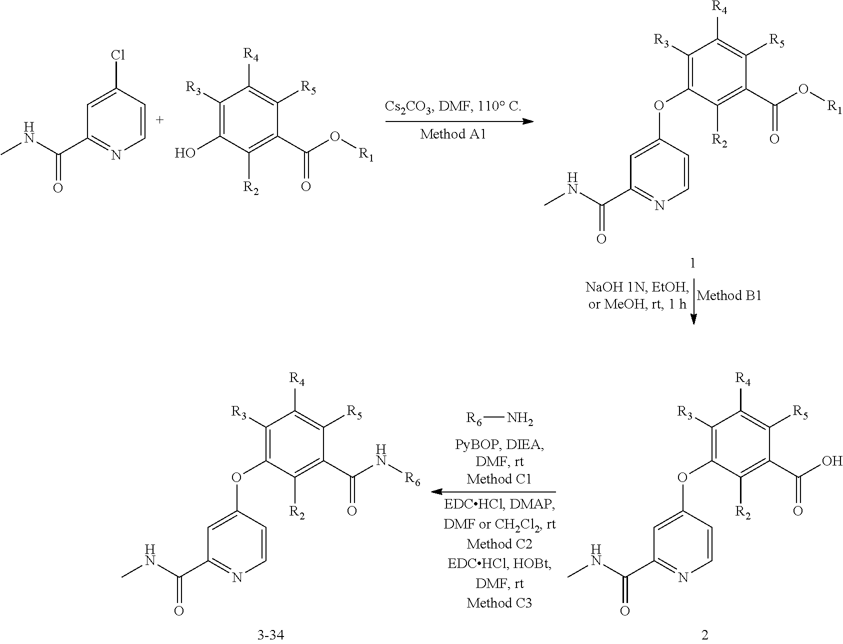

The following compound 1a is an example illustrating Method A1:

### Preparation of ethyl 3-{[2-(methylcarbamoyl)-4-pyridyl]oxy} benzoate (1a)

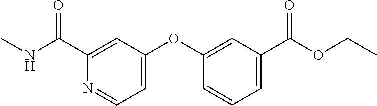 [SMILES: CCOC(=O)c1cccc(Oc2ccnc(C(=O)NC)c2)c1]

Intermediate 1a was synthesized from ethyl-3-hydroxybenzoate (6.02 mmol) and 4-chloro-N-methylpyridine-2-carboxamide (6.02 mmol) as a colorless oil in 73% yield according to the general method A1.

¹H NMR (400 MHz, CDCl₃) δ (ppm): 8.41 (d, J=5.6 Hz, 1H), 8.06 (bs, 1H), 7.96 (d, J=7.8 Hz, 1H), 7.76 (s, 1H), 7.70 (d, J=2.5 Hz, 1H), 7.51 (t, J=7.9 Hz, 1H), 7.29 (dd, J=8.1 Hz, 2.4 Hz, 1H), 6.99 (dd, J=5.6 Hz, 2.5 Hz, 1H), 4.38 (q, J=7.1 Hz, 2H), 3.01 (d, J=5.1 Hz, 3H), 1.39 (t, J=7.1 Hz, 3H).

ESI-MS: 301.50 (M+H)⁺.

The following table illustrates intermediates 1 prepared from method A1:

| Intermediate | Structure | Synthesis procedure |
|---|---|---|
| Compound 1a | 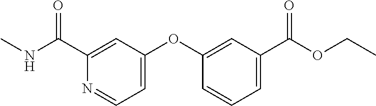 [SMILES: CCOC(=O)c1cccc(Oc2ccnc(C(=O)NC)c2)c1] | Method A1 |
| Compound 1b | 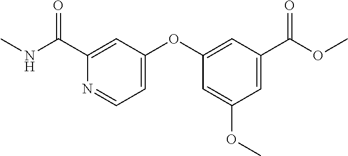 | Method A1 |
| Compound 1c | 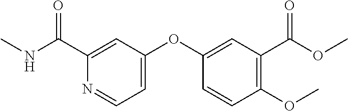 | Method A1 |

The following compound 2a is an example illustrating Method B1:

### Preparation of 3-{[2-(methylcarbamoyl)-4-pyridyl]oxy}benzoic acid (2a)

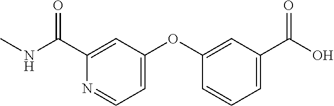 [SMILES: CNC(=O)c1cc(Oc2cccc(C(=O)O)c2)ccn1]

Intermediate 2a was synthesized from intermediate 1a (4.37 mmol) as a white powder in 97% yield according to the general method B1.

¹H NMR (400 MHz, DMSO) d (ppm): 13.26 (bs, 1H), 8.78 (q, J=4.5 Hz, 1H), 8.61-8.48 (m, 1H), 7.98-7.84 (m, 1H), 7.68-7.63 (m, 2H), 7.52 (ddd, J=8.1 Hz, 2.5 Hz, 1.0 Hz, 1H), 7.42 (d, J=2.4 Hz, 1H), 7.20 (dd, J=5.6 Hz, 2.6 Hz, 1H), 2.79 (d, J=4.9 Hz, 3H).

The following table illustrates intermediates 2 prepared from method B:

| Intermediate | Structure | Synthesis procedure |
|---|---|---|
| Compound 2a | 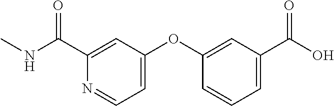 [SMILES: CNC(=O)c1cc(Oc2cccc(C(=O)O)c2)ccn1] | Method B1 |
| Compound 2b | 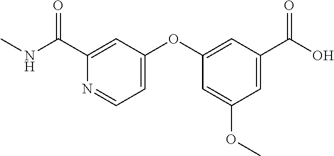 [SMILES: CNC(=O)c1cc(Oc2cc(OC)cc(C(=O)O)c2)ccn1] | Method B1 |
| Compound 2c | 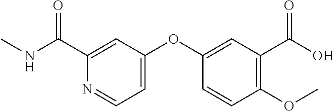 | Method B1 |

The following compounds are examples illustrating Method C: 4-[3-(benzylcarbamoyl)phenoxy]-N-methyl-pyridine-2-carboxamide (11)

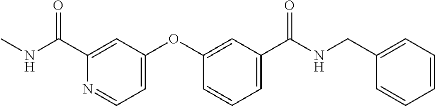 [SMILES: CNC(=O)c1cc(Oc2cccc(C(=O)NCc3ccccc3)c2)ccn1]

Compound 11 was synthesized from intermediate 2a (0.25 mmol) and benzylamine (0.30 mmol) as a white solid in 84% yield according to the general method C2. ¹H NMR (300 MHz, DMSO-d₆) δ (ppm): 9.14 (t, J=5.8 Hz, 1H), 8.80-8.77 (m, 1H), 8.54 (d, J=5.6 Hz, 1H), 7.88 (d, J=7.7 Hz, 1H), 7.72 (s, 1H), 7.63 (t, J=7.9 Hz, 1H), 7.44-7.41 (m, 2H), 7.32-7.31 (m, 4H), 7.28-7.17 (m, 2H), 4.48 (d, J=5.9 Hz, 2H), 2.79 (d, J=4.8 Hz, 3H).

### N-methyl-4-(3-{[3-(trifluoromethyl)phenyl]methylcarbamoyl}phenoxy)pyridine-2-carboxamide (20)

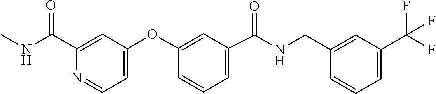 [SMILES: CNC(=O)c1cc(Oc2cccc(C(=O)NCc3cccc(C(F)(F)F)c3)c2)ccn1]

Compound 20 was synthesized from intermediate 2a (0.34 mmol) and 3-(trifluoromethyl)benzylamine (0.44 mmol) as a white solid in 79% yield according to the general method C3. ¹H NMR (600 MHz, CDCl₃) δ (ppm): δ 8.41 (d, J=5.6 Hz, 1H), 8.08 (bs, 1H), 7.70-7.65 (m, 2H), 7.60-7.45 (m, 6H), 7.26-7.24 (m, 1H), 7.01 (dd, J=5.6 Hz, 2.6 Hz, 1H), 6.67 (t, J=5.3 Hz, 1H), 4.69 (d, J=5.9 Hz, 2H), 2.99 (d, J=5.1 Hz, 3H).

ESI-MS: 430.40 (M+H)⁺.

### 4-[3-(benzylcarbamoyl)-5-methoxy-phenoxy]-N-methyl-pyridine-2-carboxamide (24)

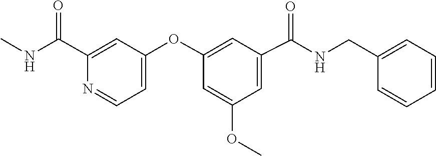 [SMILES: CNC(=O)c1cc(Oc2cc(OC)cc(C(=O)NCc3ccccc3)c2)ccn1]

Compound 24 was synthesized from intermediate 2b (0.25 mmol) and benzylamine (0.32 mmol) as a white solid in 82% yield according to the general method C3. ¹H NMR (400 MHz, CDCl₃) δ (ppm): δ 8.40 (d, J=5.2 Hz, 1H), 7.99 (bs, 1H), 7.70 (d, J=2.2 Hz, 1H), 7.38-7.28 (m, 5H), 7.25 (dd, J=2.3 Hz, 1.5 Hz, 1H), 7.04 (dd, J=2.0 Hz, 1.5 Hz, 1H), 6.97 (dd, J=5.6 Hz, 2.6 Hz, 1H), 6.76 (t, J=2.2 Hz, 1H), 6.35 (t, J=5.0 Hz, 1H), 4.63 (d, J=5.7 Hz, 2H), 3.84 (s, 3H), 3.01 (d, J=5.1 Hz, 3H).

ESI-MS: 392.30 (M+H)⁺.

### 4-[3-(benzylcarbamoyl)-4-methoxy-phenoxy]-N-methyl-pyridine-2-carboxamide (25)

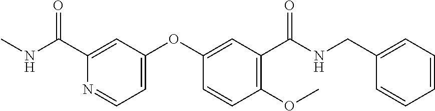 [SMILES: CNC(=O)c1cc(Oc2ccc(OC)c(C(=O)NCc3ccccc3)c2)ccn1]

Compound 25 was synthesized from intermediate 2c (0.25 mmol) and benzylamine (0.32 mmol) as a white solid in 67% yield according to the general method C3. ¹H NMR (300 MHz, CDCl₃) δ (ppm): δ 8.37 (d, J=5.6 Hz, 1H), 8.22 (t, J=4.4 Hz, 1H), 8.01-7.98 (m, 2H), 7.62 (d, J=2.5 Hz, 1H), 7.39-7.27 (m, 5H), 7.20 (dd, J=8.9 Hz, 3.1 Hz, 1H), 7.03 (d, J=8.9 Hz, 1H), 6.97 (dd, J=5.6 Hz, 2.6 Hz, 1H), 4.69 (d, J=5.7 Hz, 2H), 3.96 (s, 3H), 3.00 (d, J=5.1 Hz, 3H).

ESI-MS: 392.30 (M+H)⁺.

### Example 23: General Procedure for the Preparation of Analogues CC37-CC40

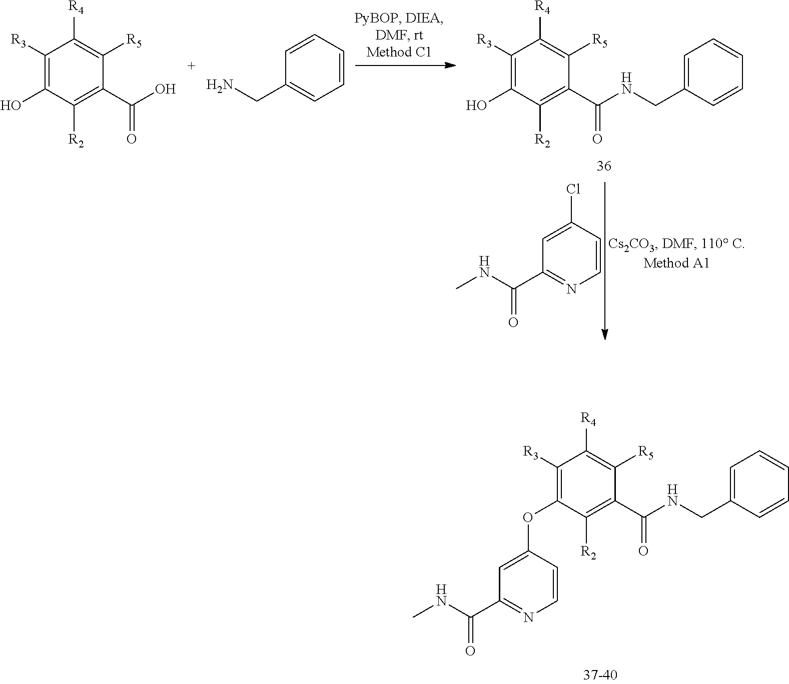

The following table illustrates intermediates 36 prepared from Method C1.

| Inter- | | Synthesis |
|---|---|---|
| mediate | Structure | procedure |
| Compound 36a | 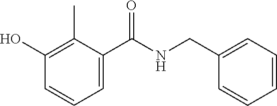 [SMILES: Cc1c(O)cccc1C(=O)NCc1ccccc1] | Method C1 |
| Compound 36b | 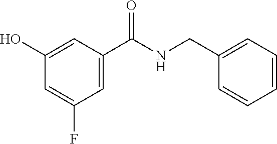 [SMILES: O=C(NCc1ccccc1)c1cc(O)cc(F)c1] | Method C1 |
| Compound 36c | 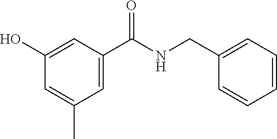 [SMILES: Cc1cc(O)cc(C(=O)NCc2ccccc2)c1] | Method C1 |
| Compound 36d | 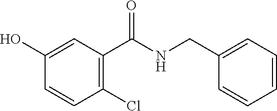 [SMILES: O=C(NCc1ccccc1)c1cc(O)ccc1Cl] | Method C1 |

The following compounds are examples illustrating the procedure A1:

### 4-[3-(benzylcarbamoyl)-2-methyl-phenoxy]-N-methyl-pyridine-2-carboxamide (37)

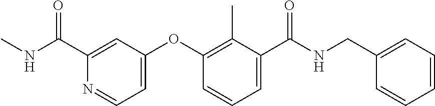 [SMILES: CNC(=O)c1cc(Oc2cccc(C(=O)NCc3ccccc3)c2C)ccn1]

Compound 37 was synthesized from intermediate 36a (0.50 mmol) and 4-chloro-N-methylpyridine-2-carboxamide (0.50 mmol) as a white solid in 15% yield according to the general method A1.

ESI-MS: 376.40 (M+H)⁺. 4-[3-(benzylcarbamoyl)-5-fluoro-phenoxy]-N-methyl-pyridine-2-carboxamide (38)

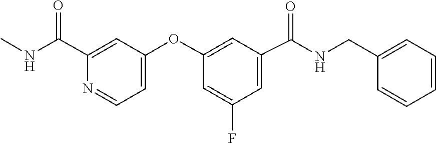 [SMILES: CNC(=O)c1cc(Oc2cc(F)cc(C(=O)NCc3ccccc3)c2)ccn1]

Compound 38 was synthesized from intermediate 36b (0.50 mmol) and 4-chloro-N-methylpyridine-2-carboxamide (0.50 mmol) as a white solid in 34% yield according to the general method A1.

ESI-MS: 380.40 (M+H)⁺.

### 4-[3-(benzylcarbamoyl)-5-methyl-phenoxy]-N-methyl-pyridine-2-carboxamide (39)

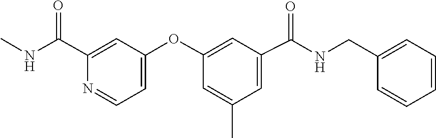 [SMILES: CNC(=O)c1cc(Oc2cc(C)cc(C(=O)NCc3ccccc3)c2)ccn1]

Compound 39 was synthesized from intermediate 36c (0.19 mmol) and 4-chloro-N-methylpyridine-2-carboxamide (0.19 mmol) as a white solid in 66% yield according to the general method A1.

ESI-MS: 376.40 (M+H)⁺.

### 4-[3-(benzylcarbamoyl)-5-methyl-phenoxy]-N-methyl-pyridine-2-carboxamide (40)

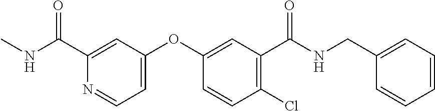 [SMILES: CNC(=O)c1cc(Oc2ccc(Cl)c(C(=O)NCc3ccccc3)c2)ccn1]

Compound 40 was synthesized from intermediate 36d (0.50 mmol) and 4-chloro-N-methylpyridine-2-carboxamide (0.50 mmol) as a white solid in 32% yield according to the general method A1.

ESI-MS: 396.40 (M+H)⁺.

TABLE 1
Examples of compounds

| CC11 | 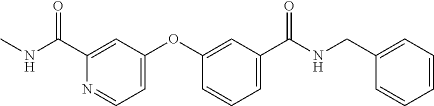 [SMILES: CNC(=O)c1cc(Oc2cccc(C(=O)NCc3ccccc3)c2)ccn1] |
|---|---|
| CC20 | 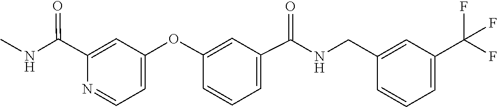 [SMILES: CNC(=O)c1cc(Oc2cccc(C(=O)NCc3cccc(C(F)(F)F)c3)c2)ccn1] |
| CC24 | 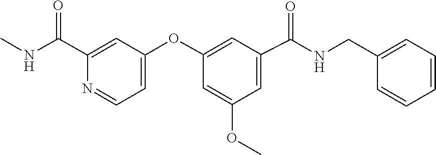 [SMILES: CNC(=O)c1cc(Oc2cc(OC)cc(C(=O)NCc3ccccc3)c2)ccn1] |
| CC25 | 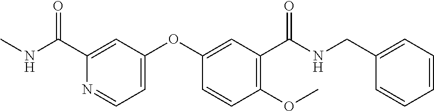 [SMILES: CNC(=O)c1cc(Oc2ccc(OC)c(C(=O)NCc3ccccc3)c2)ccn1] |
| CC37 | 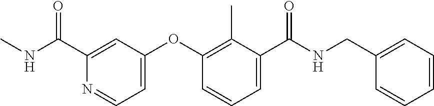 [SMILES: CNC(=O)c1cc(Oc2cccc(C(=O)NCc3ccccc3)c2C)ccn1] |
| CC38 | 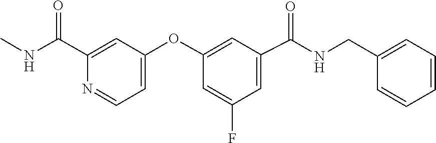 [SMILES: CNC(=O)c1cc(Oc2cc(F)cc(C(=O)NCc3ccccc3)c2)ccn1] |
| CC39 | 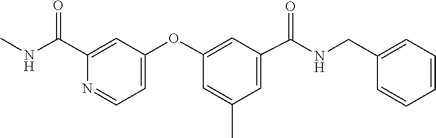 [SMILES: CNC(=O)c1cc(Oc2cc(C)cc(C(=O)NCc3ccccc3)c2)ccn1] |
| CC40 | 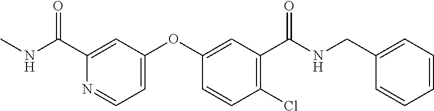 [SMILES: CNC(=O)c1cc(Oc2ccc(Cl)c(C(=O)NCc3ccccc3)c2)ccn1] |
| 44 | 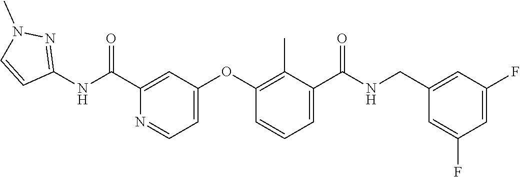 [SMILES: Cc1c(Oc2ccnc(C(=O)Nc3ccn(C)n3)c2)cccc1C(=O)NCc1cc(F)cc(F)c1] |
| 45 | 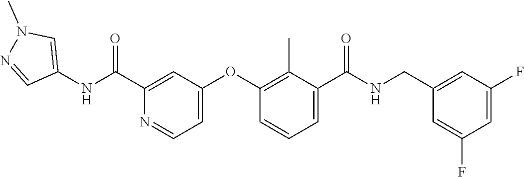 [SMILES: Cc1c(Oc2ccnc(C(=O)Nc3cnn(C)c3)c2)cccc1C(=O)NCc1cc(F)cc(F)c1] |
| 49 | 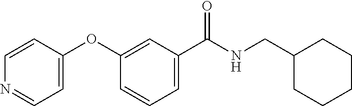 [SMILES: O=C(NCC1CCCCC1)c1cccc(Oc2ccncc2)c1] |
| 50 | 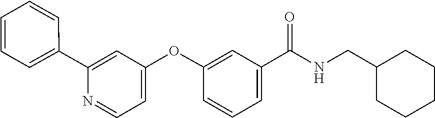 [SMILES: O=C(NCC1CCCCC1)c1cccc(Oc2ccnc(-c3ccccc3)c2)c1] |
| 68 | 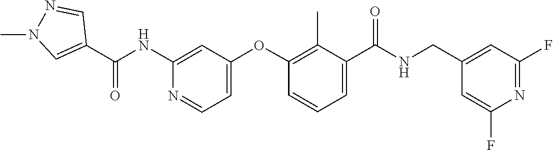 [SMILES: Cc1c(Oc2ccnc(NC(=O)c3cnn(C)c3)c2)cccc1C(=O)NCc1cc(F)nc(F)c1] |
| 69 | 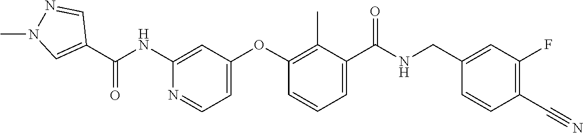 [SMILES: Cc1c(Oc2ccnc(NC(=O)c3cnn(C)c3)c2)cccc1C(=O)NCc1ccc(C#N)c(F)c1] |
| 70 | 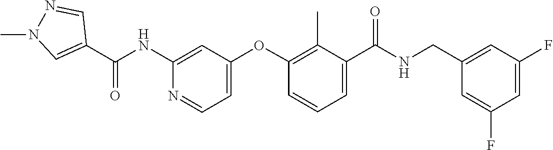 [SMILES: Cc1c(Oc2ccnc(NC(=O)c3cnn(C)c3)c2)cccc1C(=O)NCc1cc(F)cc(F)c1] |
| 71 | 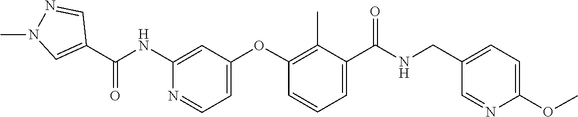 |
| 72 | 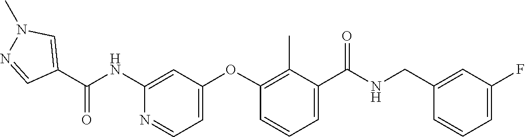 [SMILES: Cc1c(Oc2ccnc(NC(=O)c3cnn(C)c3)c2)cccc1C(=O)NCc1cccc(F)c1] |
| 73 | 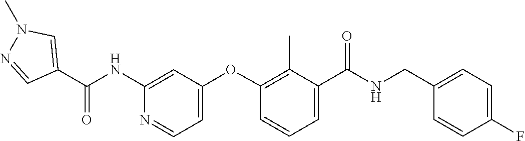 [SMILES: Cc1c(Oc2ccnc(NC(=O)c3cnn(C)c3)c2)cccc1C(=O)NCc1ccc(F)cc1] |
| 74 | 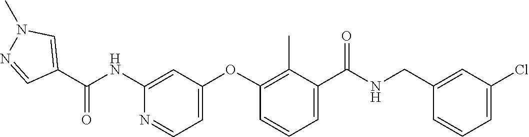 [SMILES: Cc1c(Oc2ccnc(NC(=O)c3cnn(C)c3)c2)cccc1C(=O)NCc1cccc(Cl)c1] |
| 75 | 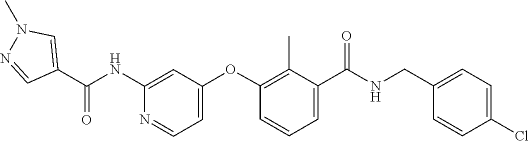 [SMILES: Cc1c(Oc2ccnc(NC(=O)c3cnn(C)c3)c2)cccc1C(=O)NCc1ccc(Cl)cc1] |
| 76 | 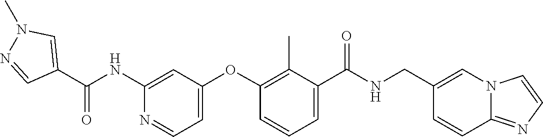 [SMILES: Cc1c(Oc2ccnc(NC(=O)c3cnn(C)c3)c2)cccc1C(=O)NCc1ccc2nccn2c1] |
| 77 | 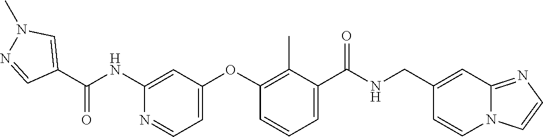 [SMILES: Cc1c(Oc2ccnc(NC(=O)c3cnn(C)c3)c2)cccc1C(=O)NCc1ccn2ccnc2c1] |
| 78 | 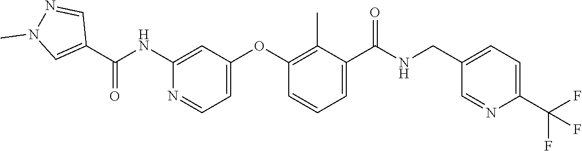 [SMILES: Cc1c(Oc2ccnc(NC(=O)c3cnn(C)c3)c2)cccc1C(=O)NCc1ccc(C(F)(F)F)nc1] |
| 79 | 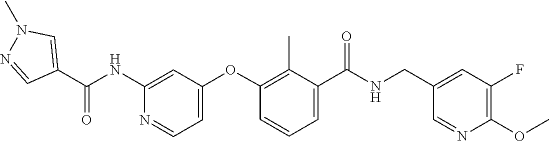 [SMILES: COc1ncc(CNC(=O)c2cccc(Oc3ccnc(NC(=O)c4cnn(C)c4)c3)c2C)cc1F] |
| 80 | 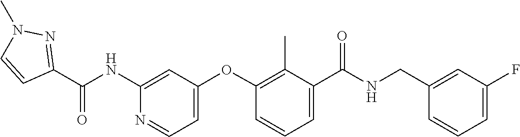 [SMILES: Cc1c(Oc2ccnc(NC(=O)c3ccn(C)n3)c2)cccc1C(=O)NCc1cccc(F)c1] |
| 81 | 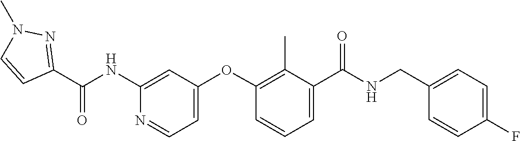 [SMILES: Cc1c(Oc2ccnc(NC(=O)c3ccn(C)n3)c2)cccc1C(=O)NCc1ccc(F)cc1] |
| 82 | 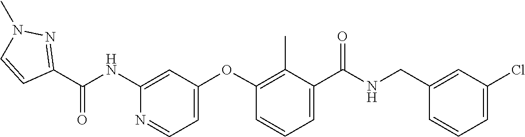 [SMILES: Cc1c(Oc2ccnc(NC(=O)c3ccn(C)n3)c2)cccc1C(=O)NCc1cccc(Cl)c1] |
| 83 | 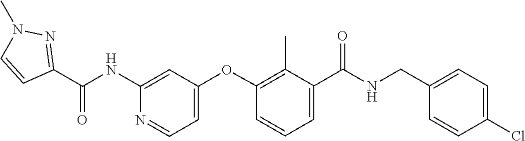 [SMILES: Cc1c(Oc2ccnc(NC(=O)c3ccn(C)n3)c2)cccc1C(=O)NCc1ccc(Cl)cc1] |
| 84 | 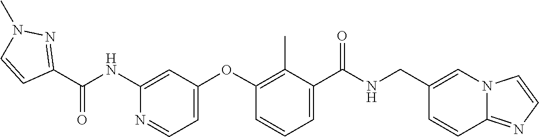 [SMILES: Cc1c(Oc2ccnc(NC(=O)c3ccn(C)n3)c2)cccc1C(=O)NCc1ccc2nccn2c1] |
| 85 | 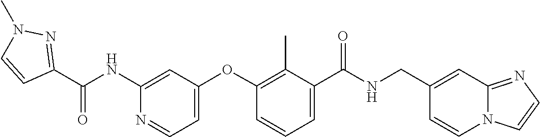 [SMILES: Cc1c(Oc2ccnc(NC(=O)c3ccn(C)n3)c2)cccc1C(=O)NCc1ccn2ccnc2c1] |
| 86 | 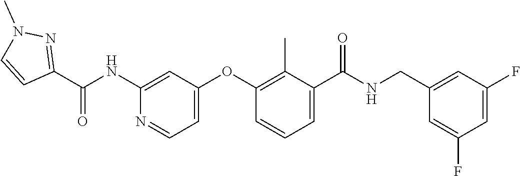 [SMILES: Cc1c(Oc2ccnc(NC(=O)c3ccn(C)n3)c2)cccc1C(=O)NCc1cc(F)cc(F)c1] |
| 87 | 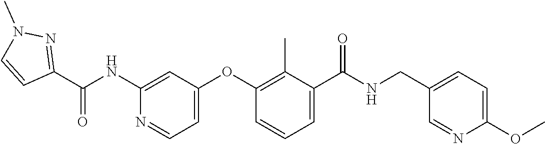 [SMILES: COc1ccc(CNC(=O)c2cccc(Oc3ccnc(NC(=O)c4ccn(C)n4)c3)c2C)cn1] |
| 88 | 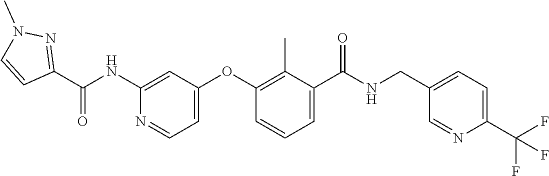 [SMILES: Cc1c(Oc2ccnc(NC(=O)c3ccn(C)n3)c2)cccc1C(=O)NCc1ccc(C(F)(F)F)nc1] |
| 89 | 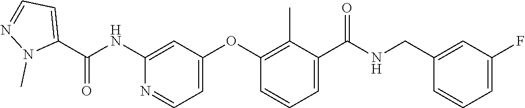 [SMILES: Cc1c(Oc2ccnc(NC(=O)c3ccnn3C)c2)cccc1C(=O)NCc1cccc(F)c1] |
| 90 | 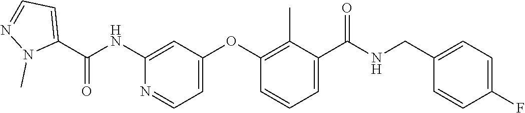 [SMILES: Cc1c(Oc2ccnc(NC(=O)c3ccnn3C)c2)cccc1C(=O)NCc1ccc(F)cc1] |
| 91 | 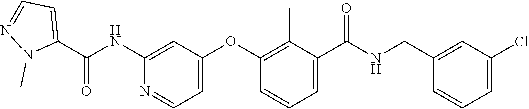 [SMILES: Cc1c(Oc2ccnc(NC(=O)c3ccnn3C)c2)cccc1C(=O)NCc1cccc(Cl)c1] |
| 92 | 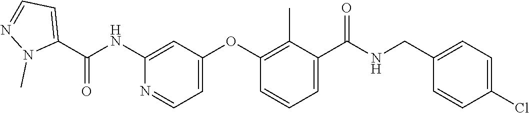 [SMILES: Cc1c(Oc2ccnc(NC(=O)c3ccnn3C)c2)cccc1C(=O)NCc1ccc(Cl)cc1] |
| 93 | 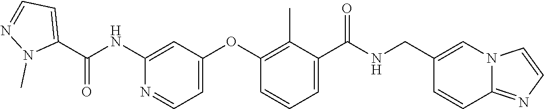 [SMILES: Cc1c(Oc2ccnc(NC(=O)c3ccnn3C)c2)cccc1C(=O)NCc1ccc2nccn2c1] |
| 94 | 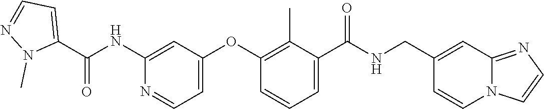 [SMILES: Cc1c(Oc2ccnc(NC(=O)c3ccnn3C)c2)cccc1C(=O)NCc1ccn2ccnc2c1] |
| 95 | 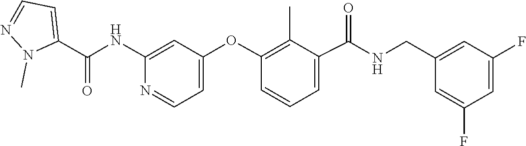 [SMILES: Cc1c(Oc2ccnc(NC(=O)c3ccnn3C)c2)cccc1C(=O)NCc1cc(F)cc(F)c1] |
| 96 | 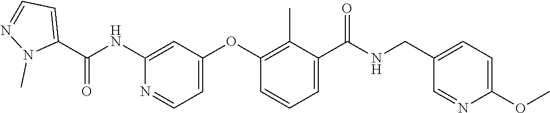 [SMILES: COc1ccc(CNC(=O)c2cccc(Oc3ccnc(NC(=O)c4ccnn4C)c3)c2C)cn1] |
| 97 | 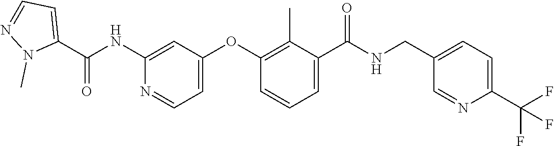 [SMILES: Cc1c(Oc2ccnc(NC(=O)c3ccnn3C)c2)cccc1C(=O)NCc1ccc(C(F)(F)F)nc1] |
| 98 | 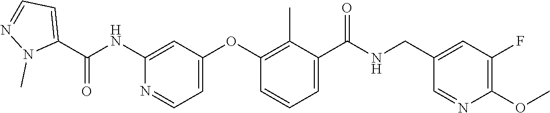 [SMILES: COc1ncc(CNC(=O)c2cccc(Oc3ccnc(NC(=O)c4ccnn4C)c3)c2C)cc1F] |
| 99 | 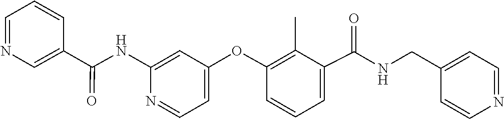 [SMILES: Cc1c(Oc2ccnc(NC(=O)c3cccnc3)c2)cccc1C(=O)NCc1ccncc1] |
| 100 | 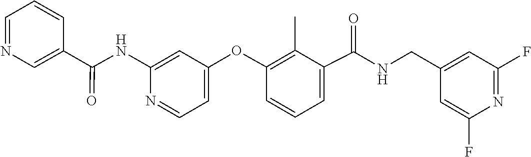 [SMILES: Cc1c(Oc2ccnc(NC(=O)c3cccnc3)c2)cccc1C(=O)NCc1cc(F)nc(F)c1] |
| 101 | 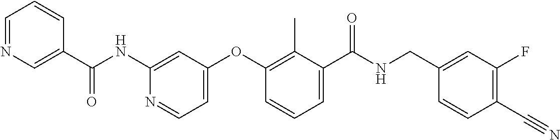 [SMILES: Cc1c(Oc2ccnc(NC(=O)c3cccnc3)c2)cccc1C(=O)NCc1ccc(C#N)c(F)c1] |
| 103 | 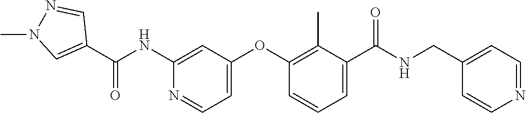 [SMILES: Cc1c(Oc2ccnc(NC(=O)c3cnn(C)c3)c2)cccc1C(=O)NCc1ccncc1] |
| 104 | 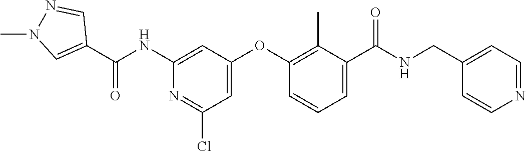 [SMILES: Cc1c(Oc2cc(Cl)nc(NC(=O)c3cnn(C)c3)c2)cccc1C(=O)NCc1ccncc1] |
| 105 | 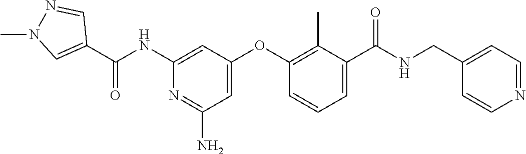 [SMILES: Cc1c(Oc2cc(N)nc(NC(=O)c3cnn(C)c3)c2)cccc1C(=O)NCc1ccncc1] |
| 106 | 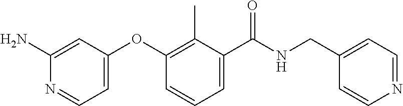 [SMILES: Cc1c(Oc2ccnc(N)c2)cccc1C(=O)NCc1ccncc1] |
| 107 | 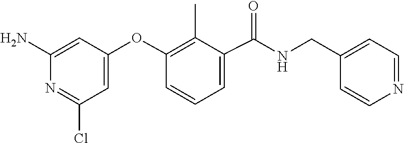 [SMILES: Cc1c(Oc2cc(N)nc(Cl)c2)cccc1C(=O)NCc1ccncc1] |
| 108 | 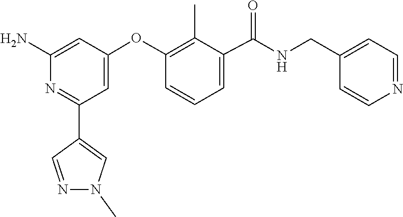 [SMILES: Cc1c(Oc2cc(N)nc(-c3cnn(C)c3)c2)cccc1C(=O)NCc1ccncc1] |
| 110 | 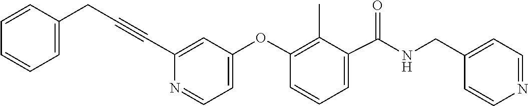 [SMILES: Cc1c(Oc2ccnc(C#CCc3ccccc3)c2)cccc1C(=O)NCc1ccncc1] |
| 111 | 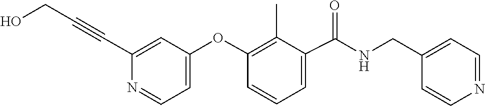 [SMILES: Cc1c(Oc2ccnc(C#CCO)c2)cccc1C(=O)NCc1ccncc1] |
| 112 | 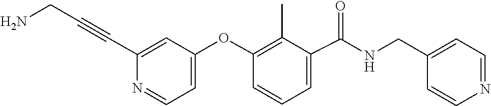 [SMILES: Cc1c(Oc2ccnc(C#CCN)c2)cccc1C(=O)NCc1ccncc1] |
| 113 | 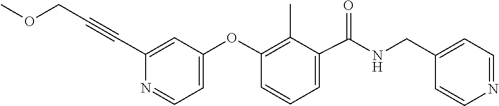 [SMILES: COCC#Cc1cc(Oc2cccc(C(=O)NCc3ccncc3)c2C)ccn1] |
| 114 | 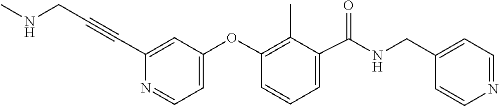 [SMILES: CNCC#Cc1cc(Oc2cccc(C(=O)NCc3ccncc3)c2C)ccn1] |
| 115 | 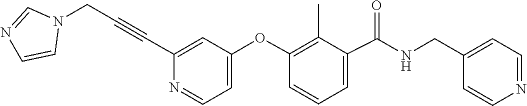 [SMILES: Cc1c(Oc2ccnc(C#CCn3ccnc3)c2)cccc1C(=O)NCc1ccncc1] |
| 116 | 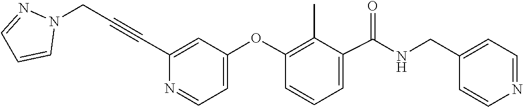 [SMILES: Cc1c(Oc2ccnc(C#CCn3cccn3)c2)cccc1C(=O)NCc1ccncc1] |
| 120 | 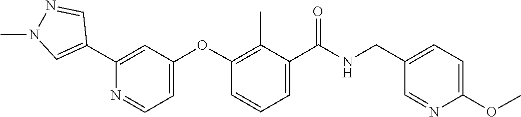 |
| 121 | 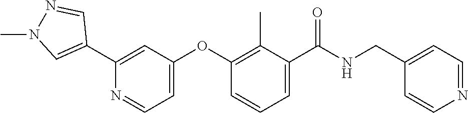 [SMILES: Cc1c(Oc2ccnc(-c3cnn(C)c3)c2)cccc1C(=O)NCc1ccncc1] |
| 122 | 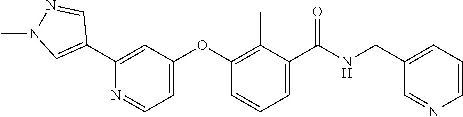 [SMILES: Cc1c(Oc2ccnc(-c3cnn(C)c3)c2)cccc1C(=O)NCc1cccnc1] |
| 123 | 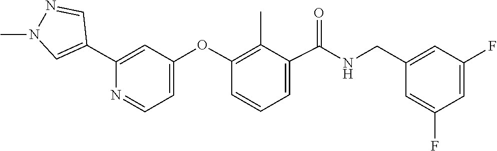 [SMILES: Cc1c(Oc2ccnc(-c3cnn(C)c3)c2)cccc1C(=O)NCc1cc(F)cc(F)c1] |
| 124 | 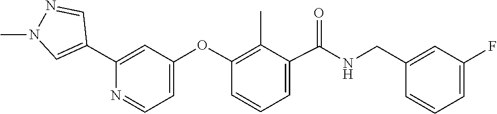 [SMILES: Cc1c(Oc2ccnc(-c3cnn(C)c3)c2)cccc1C(=O)NCc1cccc(F)c1] |
| 125 | 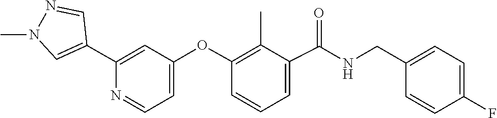 [SMILES: Cc1c(Oc2ccnc(-c3cnn(C)c3)c2)cccc1C(=O)NCc1ccc(F)cc1] |
| 126 | 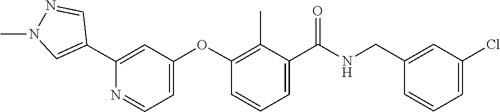 [SMILES: Cc1c(Oc2ccnc(-c3cnn(C)c3)c2)cccc1C(=O)NCc1cccc(Cl)c1] |
| 127 | 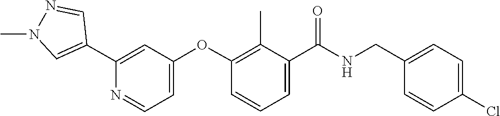 [SMILES: Cc1c(Oc2ccnc(-c3cnn(C)c3)c2)cccc1C(=O)NCc1ccc(Cl)cc1] |
| 128 | 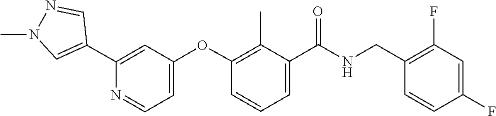 [SMILES: Cc1c(Oc2ccnc(-c3cnn(C)c3)c2)cccc1C(=O)NCc1ccc(F)cc1F] |
| 129 | 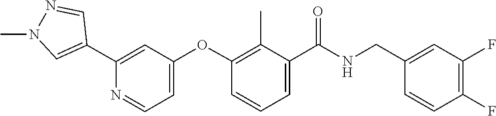 [SMILES: Cc1c(Oc2ccnc(-c3cnn(C)c3)c2)cccc1C(=O)NCc1ccc(F)c(F)c1] |
| 130 | 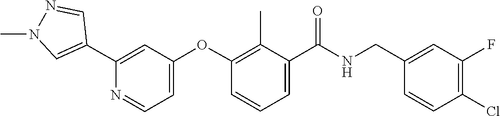 [SMILES: Cc1c(Oc2ccnc(-c3cnn(C)c3)c2)cccc1C(=O)NCc1ccc(Cl)c(F)c1] |
| 131 | 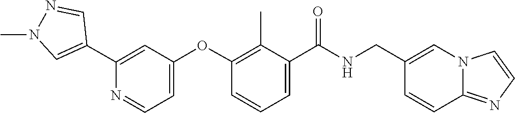 [SMILES: Cc1c(Oc2ccnc(-c3cnn(C)c3)c2)cccc1C(=O)NCc1ccc2nccn2c1] |
| 132 | 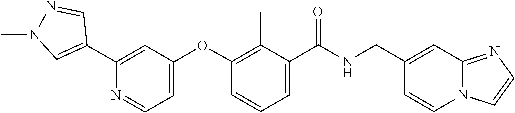 [SMILES: Cc1c(Oc2ccnc(-c3cnn(C)c3)c2)cccc1C(=O)NCc1ccn2ccnc2c1] |
| 133 | 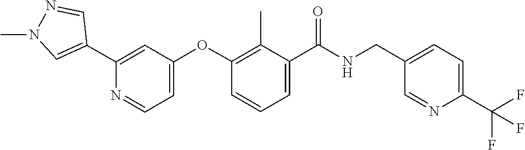 [SMILES: Cc1c(Oc2ccnc(-c3cnn(C)c3)c2)cccc1C(=O)NCc1ccc(C(F)(F)F)nc1] |
| 134 | 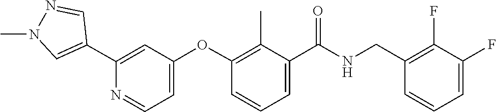 [SMILES: Cc1c(Oc2ccnc(-c3cnn(C)c3)c2)cccc1C(=O)NCc1cccc(F)c1F] |
| 135 | 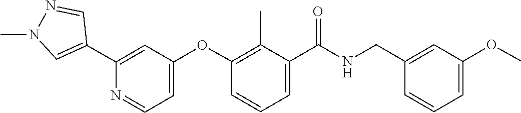 |
| 136 | 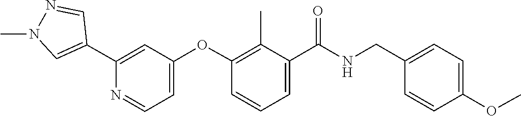 |
| 137 | 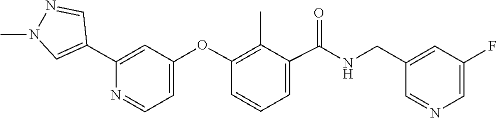 [SMILES: Cc1c(Oc2ccnc(-c3cnn(C)c3)c2)cccc1C(=O)NCc1cncc(F)c1] |
| 138 | 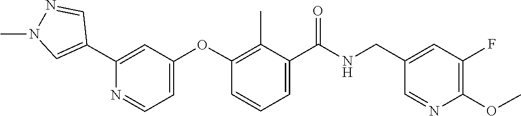 |
| 139 | 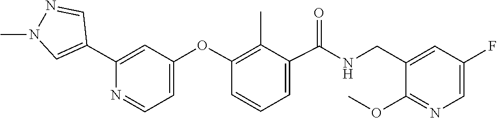 [SMILES: COc1ncc(F)cc1CNC(=O)c1cccc(Oc2ccnc(-c3cnn(C)c3)c2)c1C] |
| 140 | 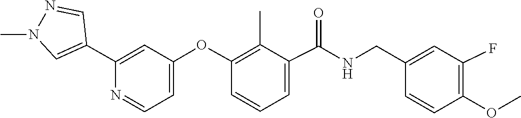 |
| 141 | 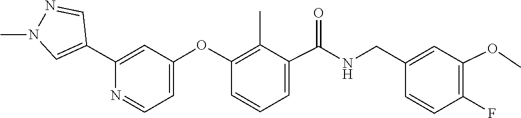 |
| 142 | 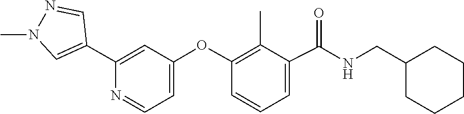 [SMILES: Cc1c(Oc2ccnc(-c3cnn(C)c3)c2)cccc1C(=O)NCC1CCCCC1] |
| 143 | 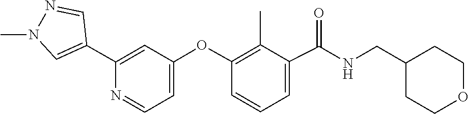 [SMILES: Cc1c(Oc2ccnc(-c3cnn(C)c3)c2)cccc1C(=O)NCC1CCOCC1] |
| 144 | 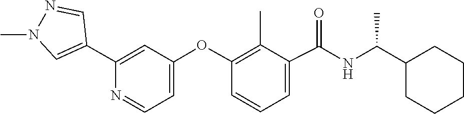 [SMILES: Cc1c(Oc2ccnc(-c3cnn(C)c3)c2)cccc1C(=O)N[C@H](C)C1CCCCC1] |
| 145 | 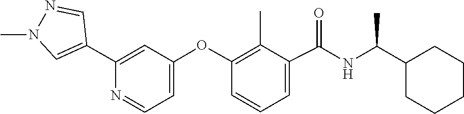 [SMILES: Cc1c(Oc2ccnc(-c3cnn(C)c3)c2)cccc1C(=O)N[C@@H](C)C1CCCCC1] |
| 146 | 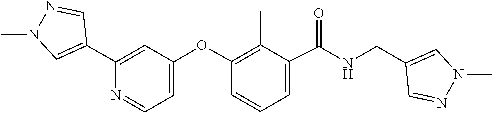 |
| 147 | 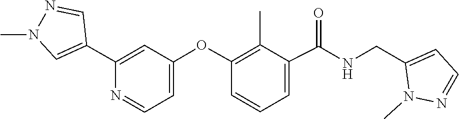 [SMILES: Cc1c(Oc2ccnc(-c3cnn(C)c3)c2)cccc1C(=O)NCc1ccnn1C] |
| 148 | 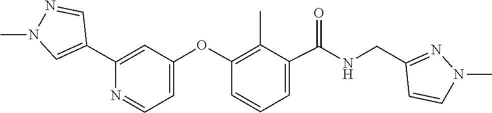 |
| 149 | 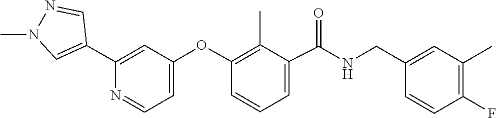 |
| 150 | 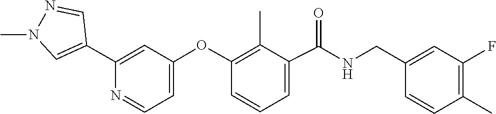 [SMILES: Cc1ccc(CNC(=O)c2cccc(Oc3ccnc(-c4cnn(C)c4)c3)c2C)cc1F] |
| 151 | 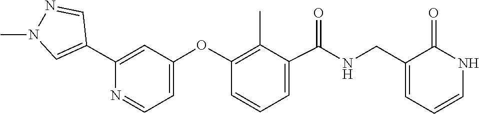 [SMILES: Cc1c(Oc2ccnc(-c3cnn(C)c3)c2)cccc1C(=O)NCc1ccc[nH]c1=O] |
| 152 | 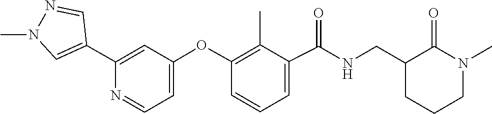 |
| 153 | 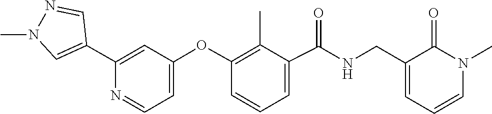 |
| 154 | 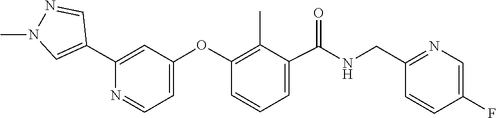 [SMILES: Cc1c(Oc2ccnc(-c3cnn(C)c3)c2)cccc1C(=O)NCc1ccc(F)cn1] |
| 155 | 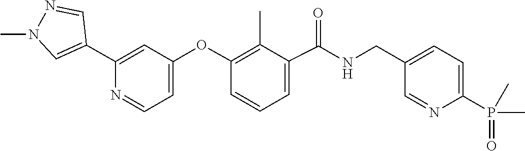 |
| 156 | 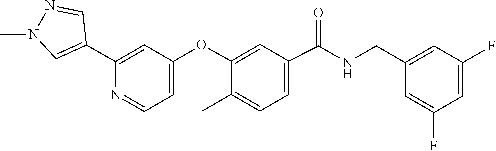 [SMILES: Cc1ccc(C(=O)NCc2cc(F)cc(F)c2)cc1Oc1ccnc(-c2cnn(C)c2)c1] |
| 157 | 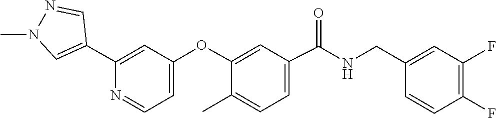 [SMILES: Cc1ccc(C(=O)NCc2ccc(F)c(F)c2)cc1Oc1ccnc(-c2cnn(C)c2)c1] |
| 158 | 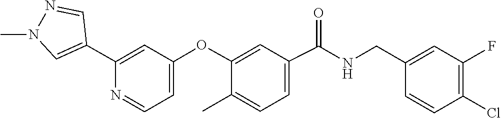 [SMILES: Cc1ccc(C(=O)NCc2ccc(Cl)c(F)c2)cc1Oc1ccnc(-c2cnn(C)c2)c1] |
| 159 | 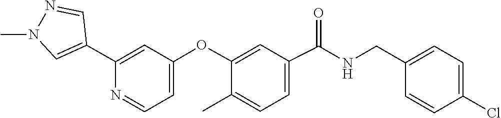 [SMILES: Cc1ccc(C(=O)NCc2ccc(Cl)cc2)cc1Oc1ccnc(-c2cnn(C)c2)c1] |
| 160 | 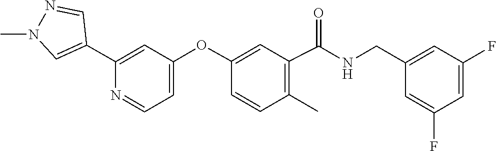 [SMILES: Cc1ccc(Oc2ccnc(-c3cnn(C)c3)c2)cc1C(=O)NCc1cc(F)cc(F)c1] |
| 161 | 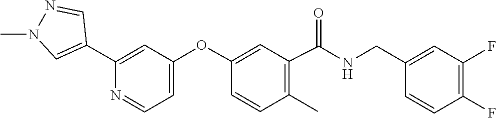 [SMILES: Cc1ccc(Oc2ccnc(-c3cnn(C)c3)c2)cc1C(=O)NCc1ccc(F)c(F)c1] |
| 162 | 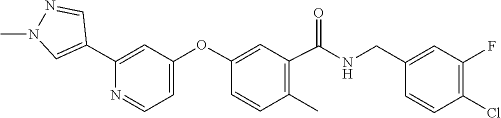 [SMILES: Cc1ccc(Oc2ccnc(-c3cnn(C)c3)c2)cc1C(=O)NCc1ccc(Cl)c(F)c1] |
| 163 | 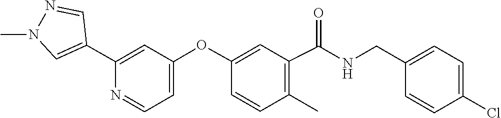 [SMILES: Cc1ccc(Oc2ccnc(-c3cnn(C)c3)c2)cc1C(=O)NCc1ccc(Cl)cc1] |
| 164 | 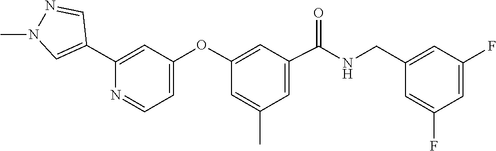 [SMILES: Cc1cc(Oc2ccnc(-c3cnn(C)c3)c2)cc(C(=O)NCc2cc(F)cc(F)c2)c1] |
| 165 | 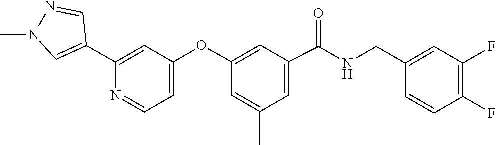 [SMILES: Cc1cc(Oc2ccnc(-c3cnn(C)c3)c2)cc(C(=O)NCc2ccc(F)c(F)c2)c1] |
| 166 | 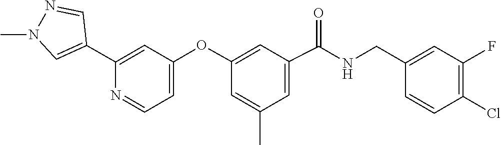 [SMILES: Cc1cc(Oc2ccnc(-c3cnn(C)c3)c2)cc(C(=O)NCc2ccc(Cl)c(F)c2)c1] |
| 167 | 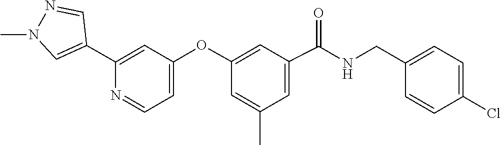 [SMILES: Cc1cc(Oc2ccnc(-c3cnn(C)c3)c2)cc(C(=O)NCc2ccc(Cl)cc2)c1] |
| 168 | 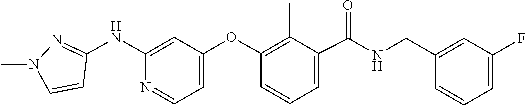 [SMILES: Cc1c(Oc2ccnc(Nc3ccn(C)n3)c2)cccc1C(=O)NCc1cccc(F)c1] |
| 169 | 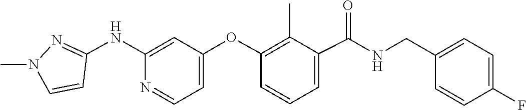 [SMILES: Cc1c(Oc2ccnc(Nc3ccn(C)n3)c2)cccc1C(=O)NCc1ccc(F)cc1] |
| 170 | 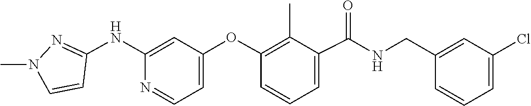 [SMILES: Cc1c(Oc2ccnc(Nc3ccn(C)n3)c2)cccc1C(=O)NCc1cccc(Cl)c1] |
| 171 | 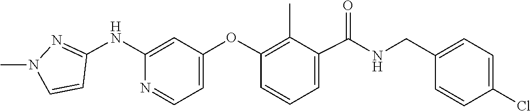 [SMILES: Cc1c(Oc2ccnc(Nc3ccn(C)n3)c2)cccc1C(=O)NCc1ccc(Cl)cc1] |
| 172 | 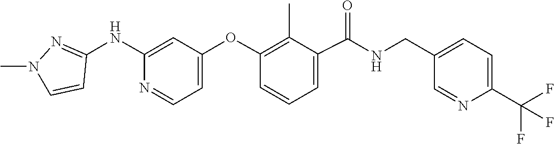 [SMILES: Cc1c(Oc2ccnc(Nc3ccn(C)n3)c2)cccc1C(=O)NCc1ccc(C(F)(F)F)nc1] |
| 173 | 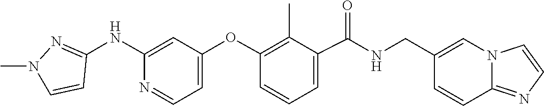 [SMILES: Cc1c(Oc2ccnc(Nc3ccn(C)n3)c2)cccc1C(=O)NCc1ccc2nccn2c1] |
| 174 | 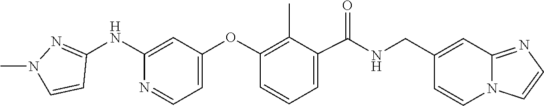 [SMILES: Cc1c(Oc2ccnc(Nc3ccn(C)n3)c2)cccc1C(=O)NCc1ccn2ccnc2c1] |
| 175 | 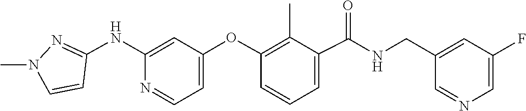 [SMILES: Cc1c(Oc2ccnc(Nc3ccn(C)n3)c2)cccc1C(=O)NCc1cncc(F)c1] |
| 176 | 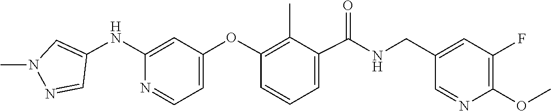 |
| 177 | 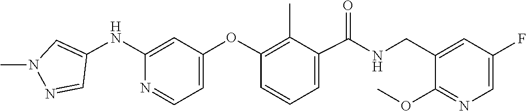 [SMILES: COc1ncc(F)cc1CNC(=O)c1cccc(Oc2ccnc(Nc3cnn(C)c3)c2)c1C] |
| 178 | 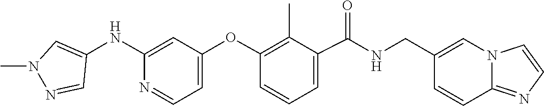 [SMILES: Cc1c(Oc2ccnc(Nc3cnn(C)c3)c2)cccc1C(=O)NCc1ccc2nccn2c1] |
| 179 | 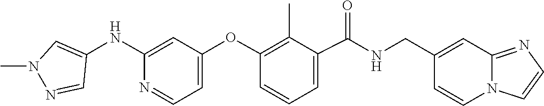 [SMILES: Cc1c(Oc2ccnc(Nc3cnn(C)c3)c2)cccc1C(=O)NCc1ccn2ccnc2c1] |
| 180 | 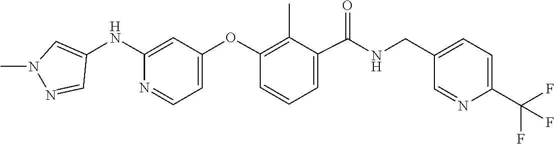 [SMILES: Cc1c(Oc2ccnc(Nc3cnn(C)c3)c2)cccc1C(=O)NCc1ccc(C(F)(F)F)nc1] |
| 181 | 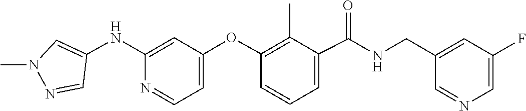 [SMILES: Cc1c(Oc2ccnc(Nc3cnn(C)c3)c2)cccc1C(=O)NCc1cncc(F)c1] |
| 182 | 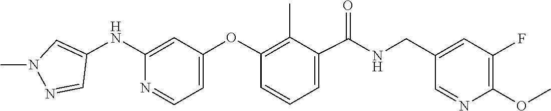 |
| 183 | 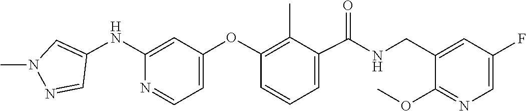 [SMILES: COc1ncc(F)cc1CNC(=O)c1cccc(Oc2ccnc(Nc3cnn(C)c3)c2)c1C] |
| 184 | 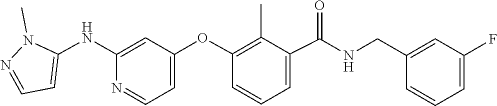 [SMILES: Cc1c(Oc2ccnc(Nc3ccnn3C)c2)cccc1C(=O)NCc1cccc(F)c1] |
| 185 | 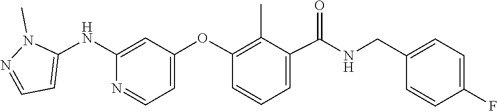 [SMILES: Cc1c(Oc2ccnc(Nc3ccnn3C)c2)cccc1C(=O)NCc1ccc(F)cc1] |
| 186 | 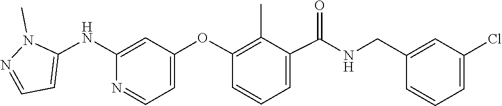 [SMILES: Cc1c(Oc2ccnc(Nc3ccnn3C)c2)cccc1C(=O)NCc1cccc(Cl)c1] |
| 187 | 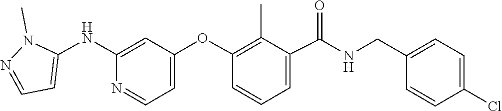 [SMILES: Cc1c(Oc2ccnc(Nc3ccnn3C)c2)cccc1C(=O)NCc1ccc(Cl)cc1] |
| 188 | 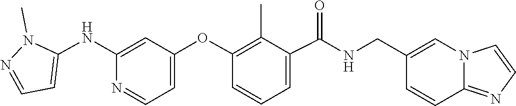 [SMILES: Cc1c(Oc2ccnc(Nc3ccnn3C)c2)cccc1C(=O)NCc1ccc2nccn2c1] |
| 189 | 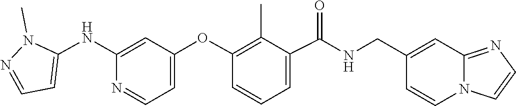 [SMILES: Cc1c(Oc2ccnc(Nc3ccnn3C)c2)cccc1C(=O)NCc1ccn2ccnc2c1] |
| 190 | 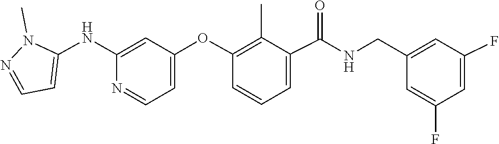 [SMILES: Cc1c(Oc2ccnc(Nc3ccnn3C)c2)cccc1C(=O)NCc1cc(F)cc(F)c1] |
| 191 | 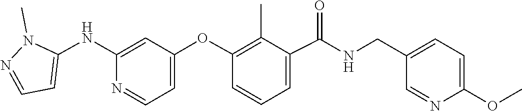 [SMILES: COc1ccc(CNC(=O)c2cccc(Oc3ccnc(Nc4ccnn4C)c3)c2C)cn1] |
| 192 | 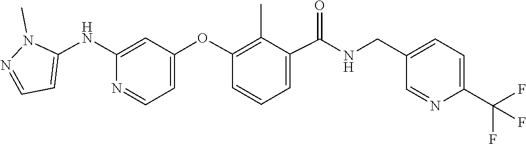 [SMILES: Cc1c(Oc2ccnc(Nc3ccnn3C)c2)cccc1C(=O)NCc1ccc(C(F)(F)F)nc1] |
| 193 | 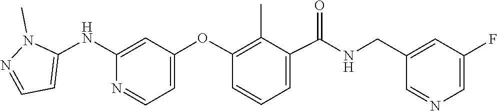 [SMILES: Cc1c(Oc2ccnc(Nc3ccnn3C)c2)cccc1C(=O)NCc1cncc(F)c1] |
| 194 | 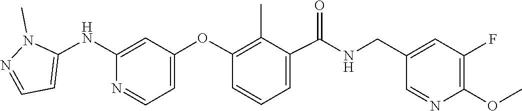 [SMILES: COc1ncc(CNC(=O)c2cccc(Oc3ccnc(Nc4ccnn4C)c3)c2C)cc1F] |
| 195 | 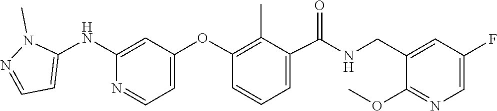 [SMILES: COc1ncc(F)cc1CNC(=O)c1cccc(Oc2ccnc(Nc3ccnn3C)c2)c1C] |
| 196 | 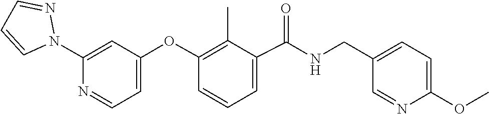 [SMILES: COc1ccc(CNC(=O)c2cccc(Oc3ccnc(-n4cccn4)c3)c2C)cn1] |
| 197 | 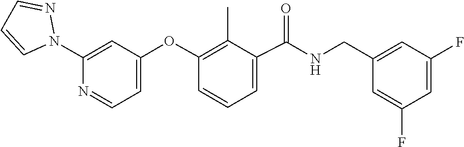 [SMILES: Cc1c(Oc2ccnc(-n3cccn3)c2)cccc1C(=O)NCc1cc(F)cc(F)c1] |
| 200 | 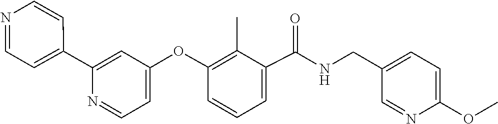 [SMILES: COc1ccc(CNC(=O)c2cccc(Oc3ccnc(-c4ccncc4)c3)c2C)cn1] |
| 201 | 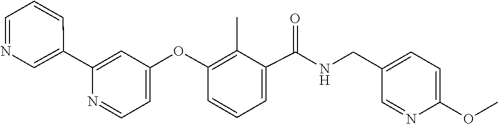 [SMILES: COc1ccc(CNC(=O)c2cccc(Oc3ccnc(-c4cccnc4)c3)c2C)cn1] |
| 202 | 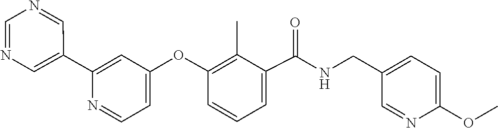 [SMILES: COc1ccc(CNC(=O)c2cccc(Oc3ccnc(-c4cncnc4)c3)c2C)cn1] |
| 203 | 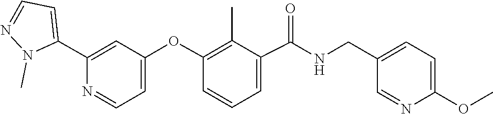 [SMILES: COc1ccc(CNC(=O)c2cccc(Oc3ccnc(-c4ccnn4C)c3)c2C)cn1] |
| 204 | 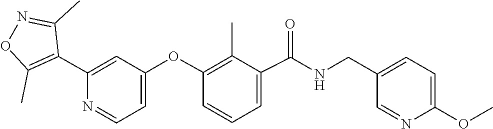 [SMILES: COc1ccc(CNC(=O)c2cccc(Oc3ccnc(-c4c(C)noc4C)c3)c2C)cn1] |
| 205 | 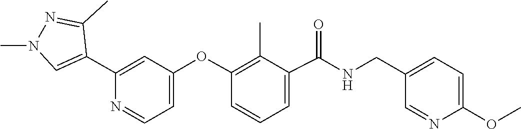 |
| 206 | 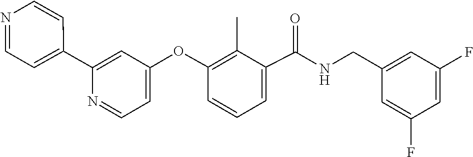 [SMILES: Cc1c(Oc2ccnc(-c3ccncc3)c2)cccc1C(=O)NCc1cc(F)cc(F)c1] |
| 207 | 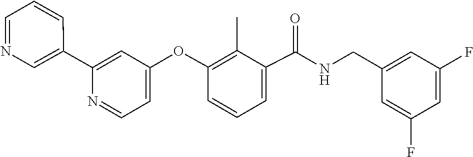 [SMILES: Cc1c(Oc2ccnc(-c3cccnc3)c2)cccc1C(=O)NCc1cc(F)cc(F)c1] |
| 208 | 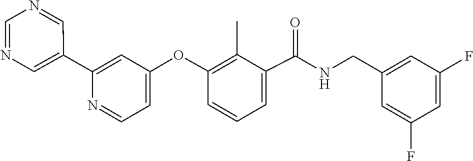 [SMILES: Cc1c(Oc2ccnc(-c3cncnc3)c2)cccc1C(=O)NCc1cc(F)cc(F)c1] |
| 209 | 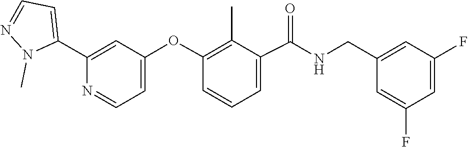 [SMILES: Cc1c(Oc2ccnc(-c3ccnn3C)c2)cccc1C(=O)NCc1cc(F)cc(F)c1] |
| 210 | 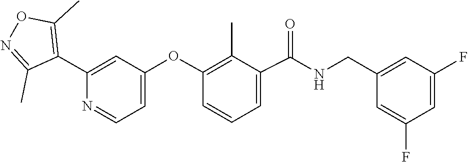 [SMILES: Cc1noc(C)c1-c1cc(Oc2cccc(C(=O)NCc3cc(F)cc(F)c3)c2C)ccn1] |
| 211 | 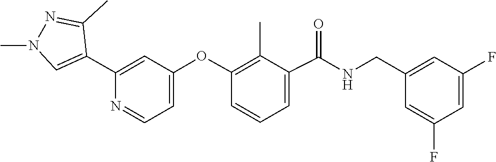 [SMILES: Cc1nn(C)cc1-c1cc(Oc2cccc(C(=O)NCc3cc(F)cc(F)c3)c2C)ccn1] |
| 212 | 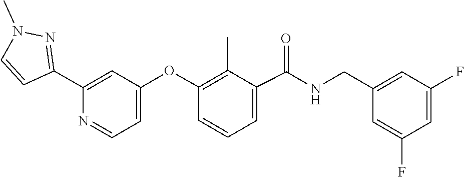 [SMILES: Cc1c(Oc2ccnc(-c3ccn(C)n3)c2)cccc1C(=O)NCc1cc(F)cc(F)c1] |
| 213 | 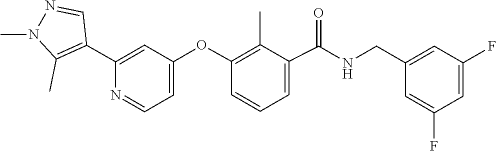 [SMILES: Cc1c(Oc2ccnc(-c3cnn(C)c3C)c2)cccc1C(=O)NCc1cc(F)cc(F)c1] |
| 214 | 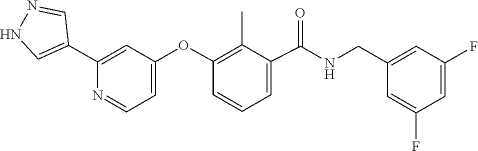 [SMILES: Cc1c(Oc2ccnc(-c3cn[nH]c3)c2)cccc1C(=O)NCc1cc(F)cc(F)c1] |
| 215 | 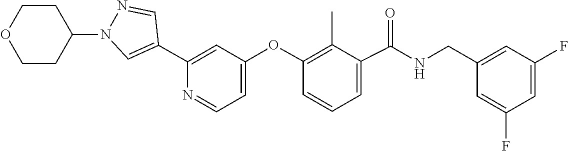 [SMILES: Cc1c(Oc2ccnc(-c3cnn(C4CCOCC4)c3)c2)cccc1C(=O)NCc1cc(F)cc(F)c1] |
| 216 | 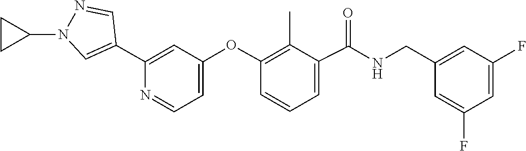 [SMILES: Cc1c(Oc2ccnc(-c3cnn(C4CC4)c3)c2)cccc1C(=O)NCc1cc(F)cc(F)c1] |
| 217 | 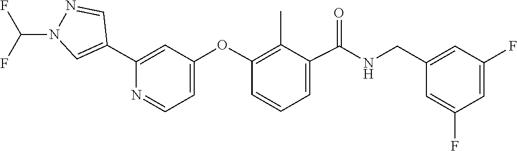 [SMILES: Cc1c(Oc2ccnc(-c3cnn(C(F)F)c3)c2)cccc1C(=O)NCc1cc(F)cc(F)c1] |
| 218 | 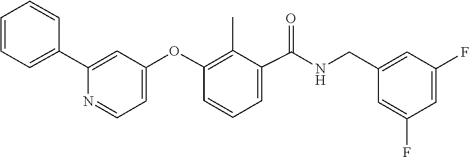 [SMILES: Cc1c(Oc2ccnc(-c3ccccc3)c2)cccc1C(=O)NCc1cc(F)cc(F)c1] |
| 219 | 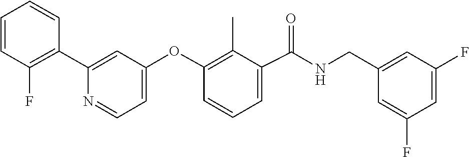 [SMILES: Cc1c(Oc2ccnc(-c3ccccc3F)c2)cccc1C(=O)NCc1cc(F)cc(F)c1] |
| 220 | 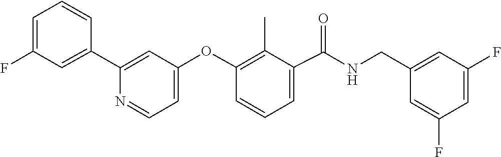 [SMILES: Cc1c(Oc2ccnc(-c3cccc(F)c3)c2)cccc1C(=O)NCc1cc(F)cc(F)c1] |
| 221 | 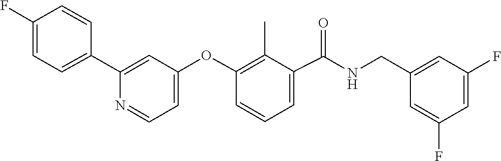 [SMILES: Cc1c(Oc2ccnc(-c3ccc(F)cc3)c2)cccc1C(=O)NCc1cc(F)cc(F)c1] |
| 222 | 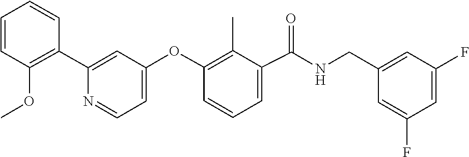 [SMILES: COc1ccccc1-c1cc(Oc2cccc(C(=O)NCc3cc(F)cc(F)c3)c2C)ccn1] |
| 223 | 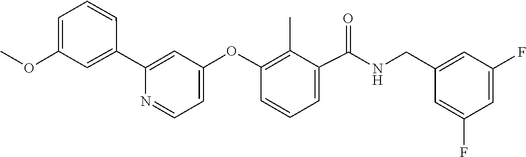 [SMILES: COc1cccc(-c2cc(Oc3cccc(C(=O)NCc4cc(F)cc(F)c4)c3C)ccn2)c1] |
| 224 | 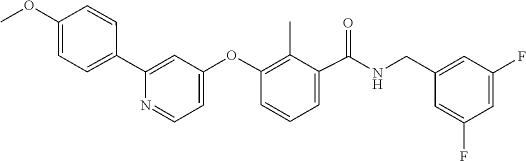 [SMILES: COc1ccc(-c2cc(Oc3cccc(C(=O)NCc4cc(F)cc(F)c4)c3C)ccn2)cc1] |
| 225 | 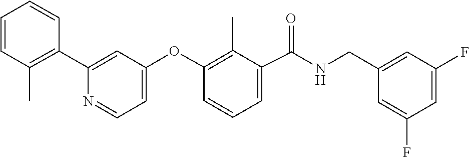 [SMILES: Cc1ccccc1-c1cc(Oc2cccc(C(=O)NCc3cc(F)cc(F)c3)c2C)ccn1] |
| 226 | 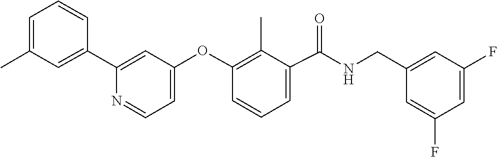 [SMILES: Cc1cccc(-c2cc(Oc3cccc(C(=O)NCc4cc(F)cc(F)c4)c3C)ccn2)c1] |
| 227 | 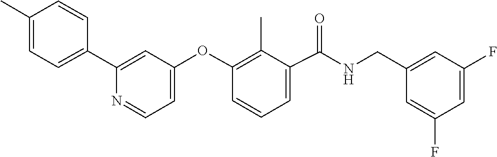 [SMILES: Cc1ccc(-c2cc(Oc3cccc(C(=O)NCc4cc(F)cc(F)c4)c3C)ccn2)cc1] |
| 228 | 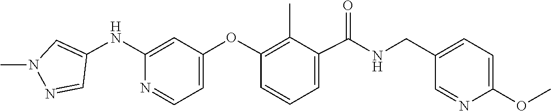 |
| 229 | 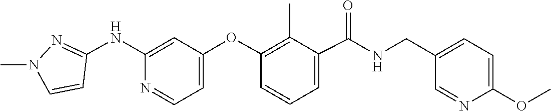 |
| 230 | 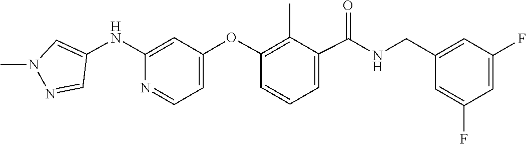 [SMILES: Cc1c(Oc2ccnc(Nc3cnn(C)c3)c2)cccc1C(=O)NCc1cc(F)cc(F)c1] |
| 231 | 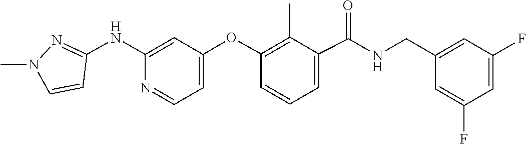 [SMILES: Cc1c(Oc2ccnc(Nc3ccn(C)n3)c2)cccc1C(=O)NCc1cc(F)cc(F)c1] |
| 232 | 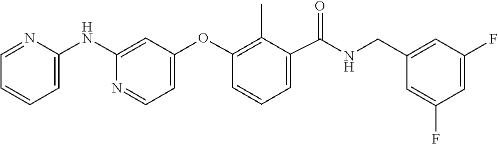 [SMILES: Cc1c(Oc2ccnc(Nc3ccccn3)c2)cccc1C(=O)NCc1cc(F)cc(F)c1] |
| 233 | 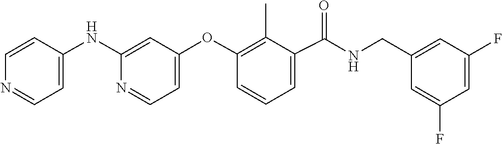 [SMILES: Cc1c(Oc2ccnc(Nc3ccncc3)c2)cccc1C(=O)NCc1cc(F)cc(F)c1] |
| 234 | 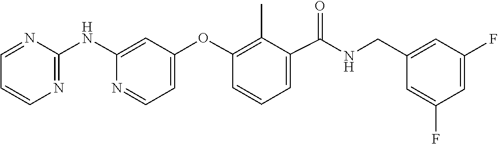 [SMILES: Cc1c(Oc2ccnc(Nc3ncccn3)c2)cccc1C(=O)NCc1cc(F)cc(F)c1] |
| 235 | 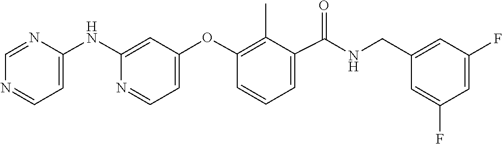 [SMILES: Cc1c(Oc2ccnc(Nc3ccncn3)c2)cccc1C(=O)NCc1cc(F)cc(F)c1] |
| 236 | 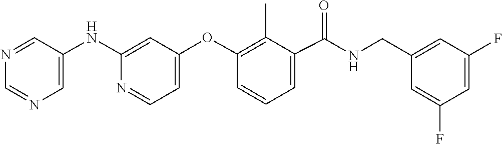 [SMILES: Cc1c(Oc2ccnc(Nc3cncnc3)c2)cccc1C(=O)NCc1cc(F)cc(F)c1] |
| 237 | 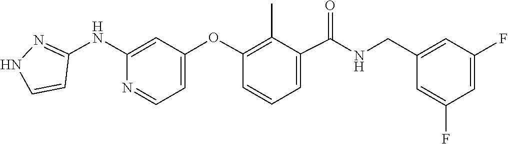 [SMILES: Cc1c(Oc2ccnc(Nc3cc[nH]n3)c2)cccc1C(=O)NCc1cc(F)cc(F)c1] |
| 238 | 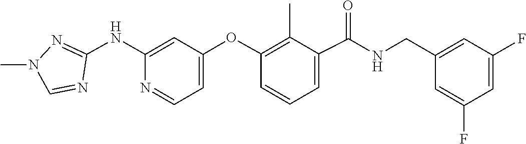 [SMILES: Cc1c(Oc2ccnc(Nc3ncn(C)n3)c2)cccc1C(=O)NCc1cc(F)cc(F)c1] |
| 239 | 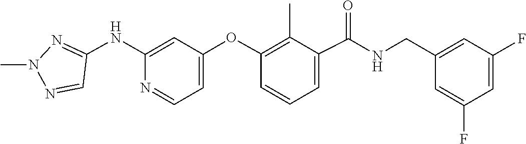 [SMILES: Cc1c(Oc2ccnc(Nc3cnn(C)n3)c2)cccc1C(=O)NCc1cc(F)cc(F)c1] |
| 240 | 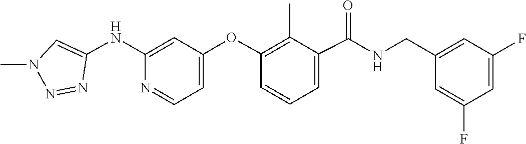 [SMILES: Cc1c(Oc2ccnc(Nc3cn(C)nn3)c2)cccc1C(=O)NCc1cc(F)cc(F)c1] |
| 241 | 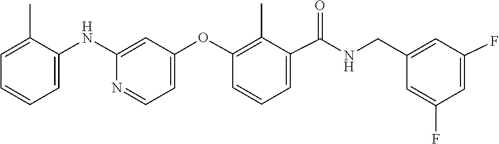 [SMILES: Cc1ccccc1Nc1cc(Oc2cccc(C(=O)NCc3cc(F)cc(F)c3)c2C)ccn1] |
| 242 | 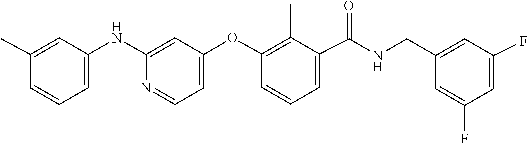 [SMILES: Cc1cccc(Nc2cc(Oc3cccc(C(=O)NCc4cc(F)cc(F)c4)c3C)ccn2)c1] |
| 243 | 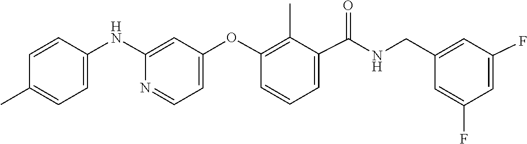 [SMILES: Cc1ccc(Nc2cc(Oc3cccc(C(=O)NCc4cc(F)cc(F)c4)c3C)ccn2)cc1] |
| 244 | 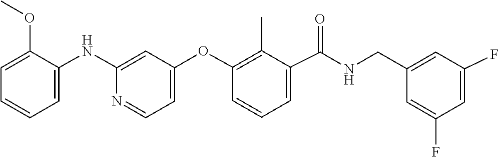 [SMILES: COc1ccccc1Nc1cc(Oc2cccc(C(=O)NCc3cc(F)cc(F)c3)c2C)ccn1] |
| 245 | 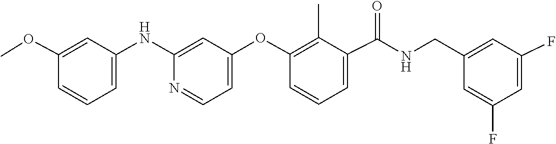 [SMILES: COc1cccc(Nc2cc(Oc3cccc(C(=O)NCc4cc(F)cc(F)c4)c3C)ccn2)c1] |
| 246 | 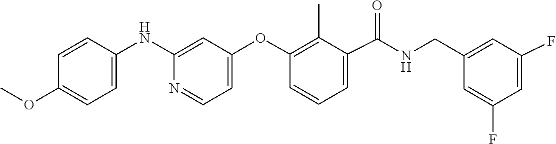 [SMILES: COc1ccc(Nc2cc(Oc3cccc(C(=O)NCc4cc(F)cc(F)c4)c3C)ccn2)cc1] |
| 247 | 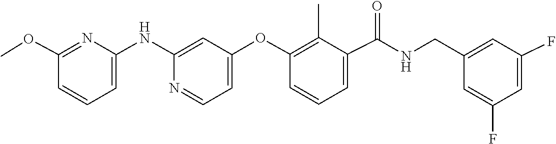 [SMILES: COc1cccc(Nc2cc(Oc3cccc(C(=O)NCc4cc(F)cc(F)c4)c3C)ccn2)n1] |
| 248 | 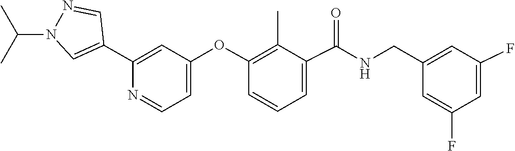 [SMILES: Cc1c(Oc2ccnc(-c3cnn(C(C)C)c3)c2)cccc1C(=O)NCc1cc(F)cc(F)c1] |
| 249 | 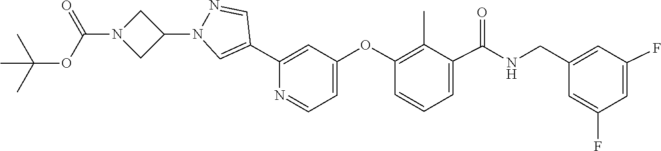 [SMILES: Cc1c(Oc2ccnc(-c3cnn(C4CN(C(=O)OC(C)(C)C)C4)c3)c2)cccc1C(=O)NCc1cc(F)cc(F)c1] |
| 250 | 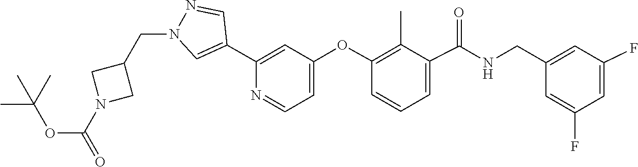 [SMILES: Cc1c(Oc2ccnc(-c3cnn(CC4CN(C(=O)OC(C)(C)C)C4)c3)c2)cccc1C(=O)NCc1cc(F)cc(F)c1] |
| 251 | 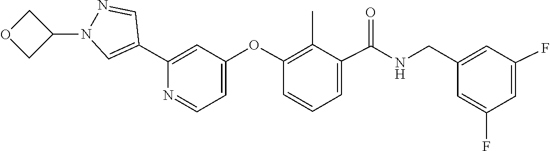 [SMILES: Cc1c(Oc2ccnc(-c3cnn(C4COC4)c3)c2)cccc1C(=O)NCc1cc(F)cc(F)c1] |
| 252 | 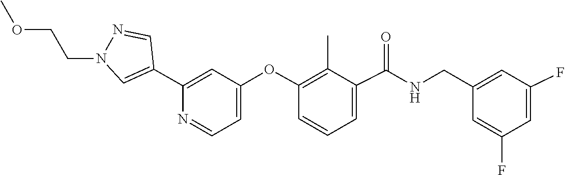 [SMILES: COCCn1cc(-c2cc(Oc3cccc(C(=O)NCc4cc(F)cc(F)c4)c3C)ccn2)cn1] |
| 253 | 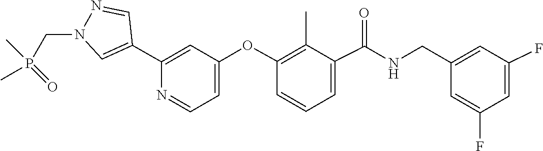 [SMILES: Cc1c(Oc2ccnc(-c3cnn(CP(C)(C)=O)c3)c2)cccc1C(=O)NCc1cc(F)cc(F)c1] |
| 254 | 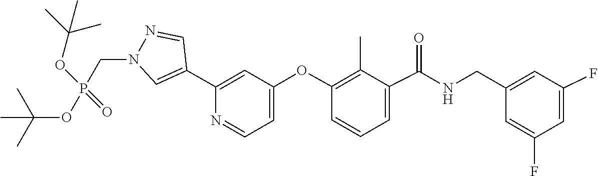 [SMILES: Cc1c(Oc2ccnc(-c3cnn(CP(=O)(OC(C)(C)C)OC(C)(C)C)c3)c2)cccc1C(=O)NCc1cc(F)cc(F)c1] |
| 255 | 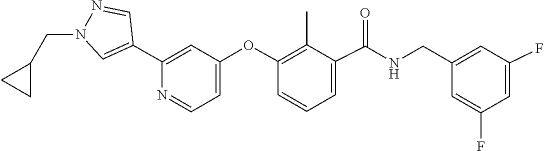 [SMILES: Cc1c(Oc2ccnc(-c3cnn(CC4CC4)c3)c2)cccc1C(=O)NCc1cc(F)cc(F)c1] |
| 256 | 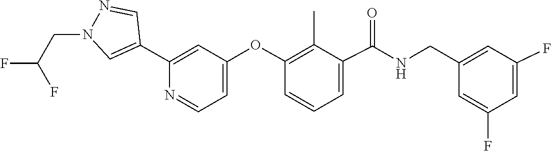 [SMILES: Cc1c(Oc2ccnc(-c3cnn(CC(F)F)c3)c2)cccc1C(=O)NCc1cc(F)cc(F)c1] |
| 257 | 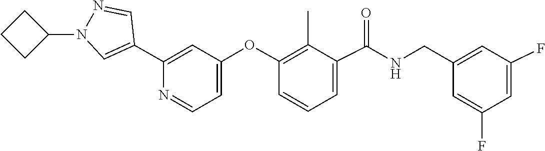 [SMILES: Cc1c(Oc2ccnc(-c3cnn(C4CCC4)c3)c2)cccc1C(=O)NCc1cc(F)cc(F)c1] |
| 258 | 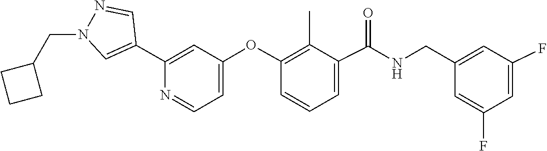 [SMILES: Cc1c(Oc2ccnc(-c3cnn(CC4CCC4)c3)c2)cccc1C(=O)NCc1cc(F)cc(F)c1] |
| 259 | 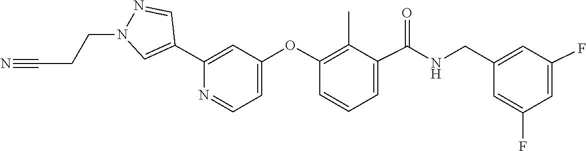 [SMILES: Cc1c(Oc2ccnc(-c3cnn(CCC#N)c3)c2)cccc1C(=O)NCc1cc(F)cc(F)c1] |
| 260 | 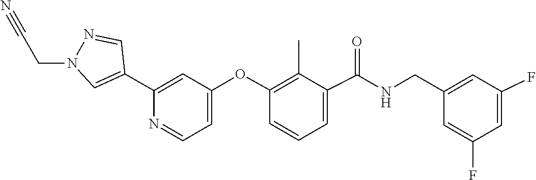 [SMILES: Cc1c(Oc2ccnc(-c3cnn(CC#N)c3)c2)cccc1C(=O)NCc1cc(F)cc(F)c1] |
| 261 | 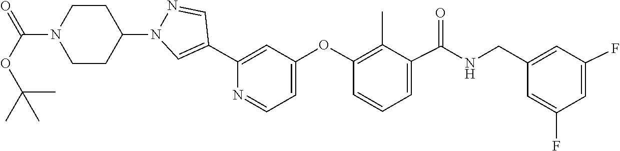 [SMILES: Cc1c(Oc2ccnc(-c3cnn(C4CCN(C(=O)OC(C)(C)C)CC4)c3)c2)cccc1C(=O)NCc1cc(F)cc(F)c1] |
| 262 | 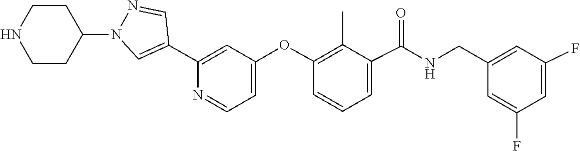 [SMILES: Cc1c(Oc2ccnc(-c3cnn(C4CCNCC4)c3)c2)cccc1C(=O)NCc1cc(F)cc(F)c1] |
| 263 | 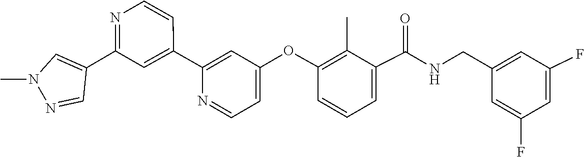 [SMILES: Cc1c(Oc2ccnc(-c3ccnc(-c4cnn(C)c4)c3)c2)cccc1C(=O)NCc1cc(F)cc(F)c1] |
| 264 | 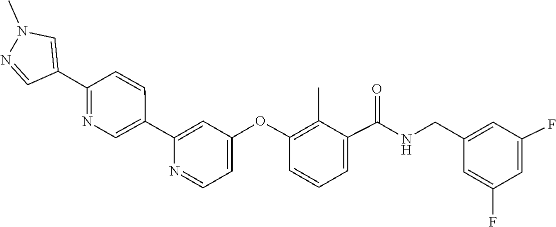 [SMILES: Cc1c(Oc2ccnc(-c3ccc(-c4cnn(C)c4)nc3)c2)cccc1C(=O)NCc1cc(F)cc(F)c1] |
| 265a | 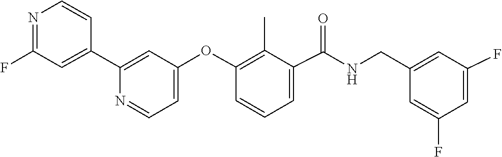 [SMILES: Cc1c(Oc2ccnc(-c3ccnc(F)c3)c2)cccc1C(=O)NCc1cc(F)cc(F)c1] |
| 265b | 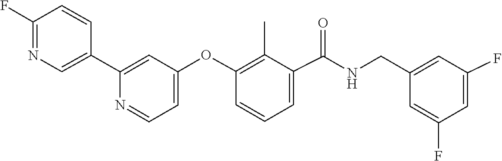 [SMILES: Cc1c(Oc2ccnc(-c3ccc(F)nc3)c2)cccc1C(=O)NCc1cc(F)cc(F)c1] |
| 266 | 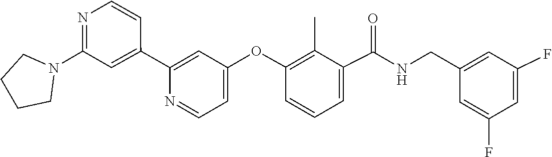 [SMILES: Cc1c(Oc2ccnc(-c3ccnc(N4CCCC4)c3)c2)cccc1C(=O)NCc1cc(F)cc(F)c1] |
| 267 | 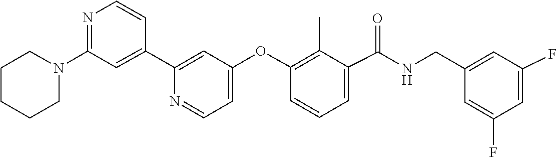 [SMILES: Cc1c(Oc2ccnc(-c3ccnc(N4CCCCC4)c3)c2)cccc1C(=O)NCc1cc(F)cc(F)c1] |
| 268 | 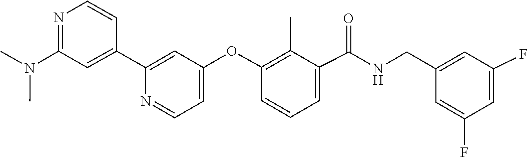 [SMILES: Cc1c(Oc2ccnc(-c3ccnc(N(C)C)c3)c2)cccc1C(=O)NCc1cc(F)cc(F)c1] |
| 269 | 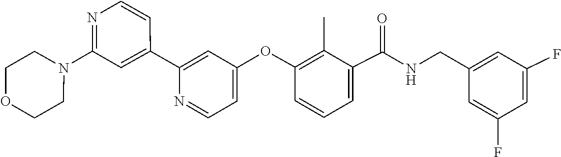 [SMILES: Cc1c(Oc2ccnc(-c3ccnc(N4CCOCC4)c3)c2)cccc1C(=O)NCc1cc(F)cc(F)c1] |
| 270 | 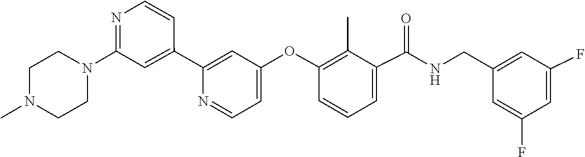 [SMILES: Cc1c(Oc2ccnc(-c3ccnc(N4CCN(C)CC4)c3)c2)cccc1C(=O)NCc1cc(F)cc(F)c1] |
| 271 | 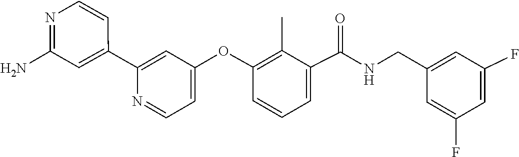 [SMILES: Cc1c(Oc2ccnc(-c3ccnc(N)c3)c2)cccc1C(=O)NCc1cc(F)cc(F)c1] |
| 272 | 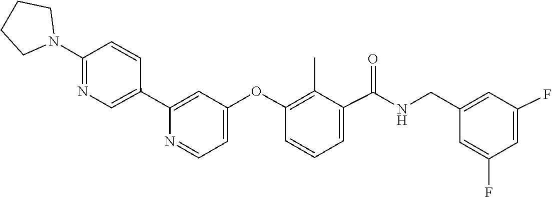 [SMILES: Cc1c(Oc2ccnc(-c3ccc(N4CCCC4)nc3)c2)cccc1C(=O)NCc1cc(F)cc(F)c1] |
| 273 | 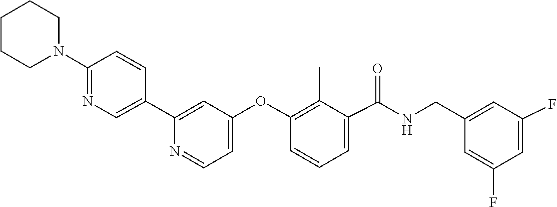 [SMILES: Cc1c(Oc2ccnc(-c3ccc(N4CCCCC4)nc3)c2)cccc1C(=O)NCc1cc(F)cc(F)c1] |
| 274 | 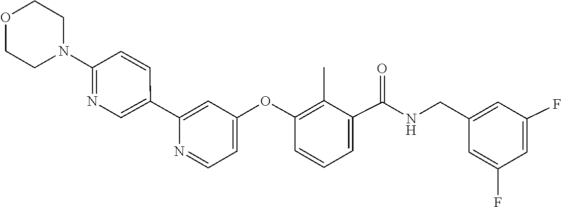 [SMILES: Cc1c(Oc2ccnc(-c3ccc(N4CCOCC4)nc3)c2)cccc1C(=O)NCc1cc(F)cc(F)c1] |
| 275 | 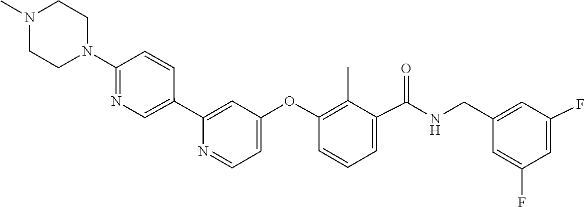 [SMILES: Cc1c(Oc2ccnc(-c3ccc(N4CCN(C)CC4)nc3)c2)cccc1C(=O)NCc1cc(F)cc(F)c1] |
| 276 | 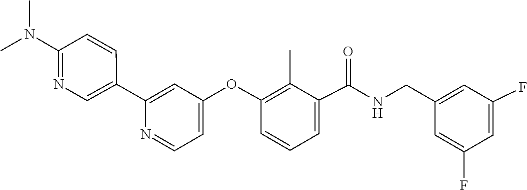 [SMILES: Cc1c(Oc2ccnc(-c3ccc(N(C)C)nc3)c2)cccc1C(=O)NCc1cc(F)cc(F)c1] |
| 277 | 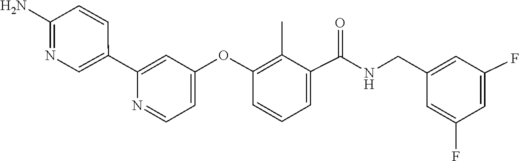 [SMILES: Cc1c(Oc2ccnc(-c3ccc(N)nc3)c2)cccc1C(=O)NCc1cc(F)cc(F)c1] |
| 278 | 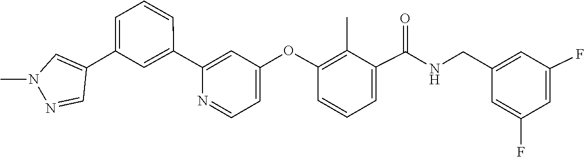 [SMILES: Cc1c(Oc2ccnc(-c3cccc(-c4cnn(C)c4)c3)c2)cccc1C(=O)NCc1cc(F)cc(F)c1] |
| 279 | 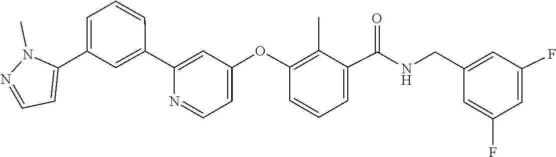 [SMILES: Cc1c(Oc2ccnc(-c3cccc(-c4ccnn4C)c3)c2)cccc1C(=O)NCc1cc(F)cc(F)c1] |
| 280 | 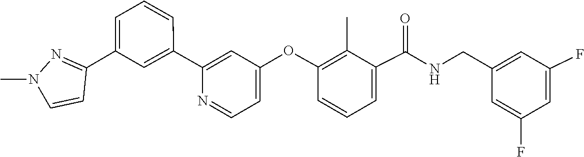 [SMILES: Cc1c(Oc2ccnc(-c3cccc(-c4ccn(C)n4)c3)c2)cccc1C(=O)NCc1cc(F)cc(F)c1] |
| 281 | 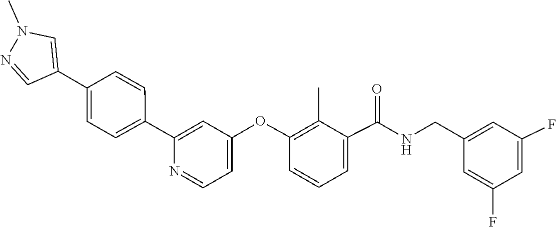 [SMILES: Cc1c(Oc2ccnc(-c3ccc(-c4cnn(C)c4)cc3)c2)cccc1C(=O)NCc1cc(F)cc(F)c1] |
| 282 | 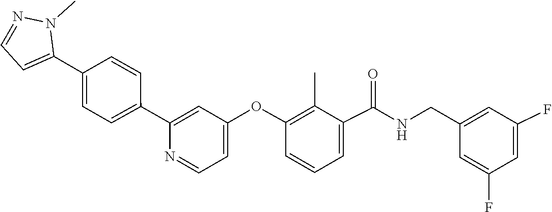 [SMILES: Cc1c(Oc2ccnc(-c3ccc(-c4ccnn4C)cc3)c2)cccc1C(=O)NCc1cc(F)cc(F)c1] |
| 283 | 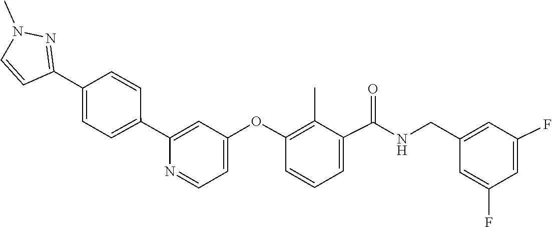 [SMILES: Cc1c(Oc2ccnc(-c3ccc(-c4ccn(C)n4)cc3)c2)cccc1C(=O)NCc1cc(F)cc(F)c1] |
| 285 | 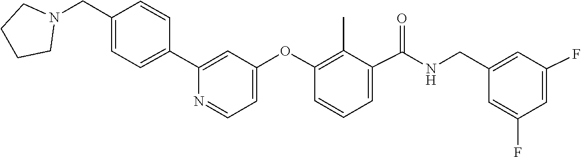 [SMILES: Cc1c(Oc2ccnc(-c3ccc(CN4CCCC4)cc3)c2)cccc1C(=O)NCc1cc(F)cc(F)c1] |
| 286 | 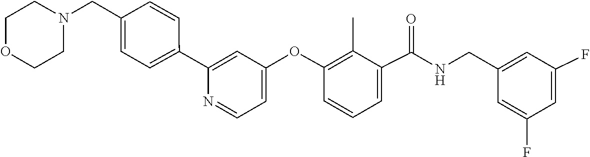 [SMILES: Cc1c(Oc2ccnc(-c3ccc(CN4CCOCC4)cc3)c2)cccc1C(=O)NCc1cc(F)cc(F)c1] |
| 287 | 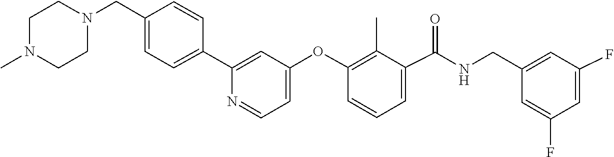 [SMILES: Cc1c(Oc2ccnc(-c3ccc(CN4CCN(C)CC4)cc3)c2)cccc1C(=O)NCc1cc(F)cc(F)c1] |
| 288 | 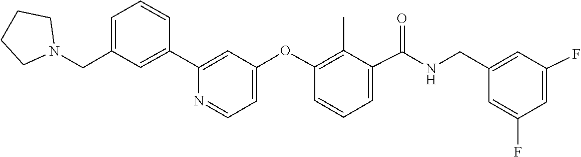 [SMILES: Cc1c(Oc2ccnc(-c3cccc(CN4CCCC4)c3)c2)cccc1C(=O)NCc1cc(F)cc(F)c1] |
| 289 | 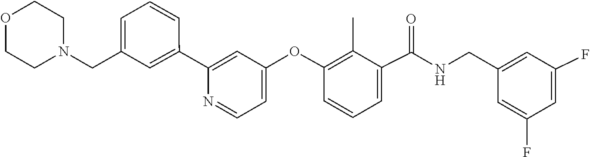 [SMILES: Cc1c(Oc2ccnc(-c3cccc(CN4CCOCC4)c3)c2)cccc1C(=O)NCc1cc(F)cc(F)c1] |
| 290 | 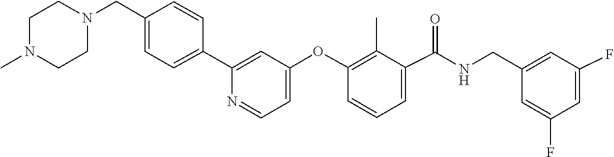 [SMILES: Cc1c(Oc2ccnc(-c3ccc(CN4CCN(C)CC4)cc3)c2)cccc1C(=O)NCc1cc(F)cc(F)c1] |
| 292 | 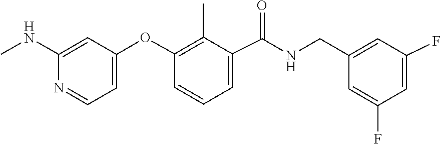 [SMILES: CNc1cc(Oc2cccc(C(=O)NCc3cc(F)cc(F)c3)c2C)ccn1] |
| 293 | 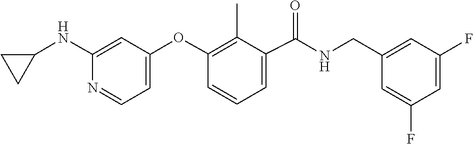 [SMILES: Cc1c(Oc2ccnc(NC3CC3)c2)cccc1C(=O)NCc1cc(F)cc(F)c1] |
| 294 | 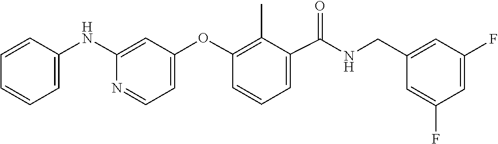 [SMILES: Cc1c(Oc2ccnc(Nc3ccccc3)c2)cccc1C(=O)NCc1cc(F)cc(F)c1] |
| 295 | 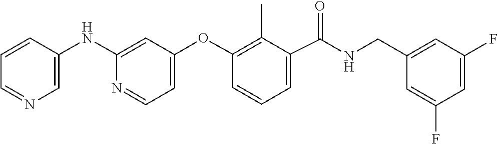 [SMILES: Cc1c(Oc2ccnc(Nc3cccnc3)c2)cccc1C(=O)NCc1cc(F)cc(F)c1] |
| 296 | 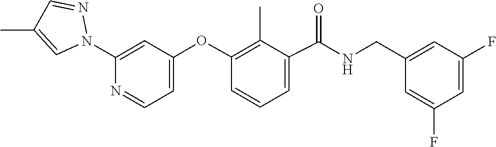 [SMILES: Cc1cnn(-c2cc(Oc3cccc(C(=O)NCc4cc(F)cc(F)c4)c3C)ccn2)c1] |
| 297 | 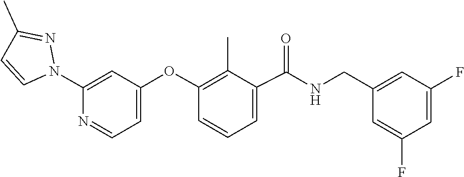 [SMILES: Cc1ccn(-c2cc(Oc3cccc(C(=O)NCc4cc(F)cc(F)c4)c3C)ccn2)n1] |
| 302 | 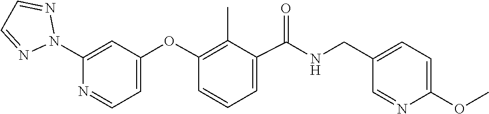 [SMILES: COc1ccc(CNC(=O)c2cccc(Oc3ccnc(-n4nccn4)c3)c2C)cn1] |
| 303 | 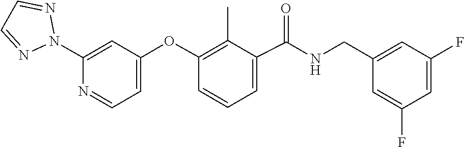 [SMILES: Cc1c(Oc2ccnc(-n3nccn3)c2)cccc1C(=O)NCc1cc(F)cc(F)c1] |
| 304 | 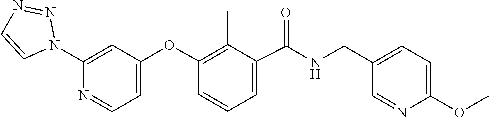 [SMILES: COc1ccc(CNC(=O)c2cccc(Oc3ccnc(-n4ccnn4)c3)c2C)cn1] |
| 305 | 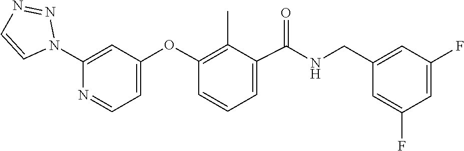 [SMILES: Cc1c(Oc2ccnc(-n3ccnn3)c2)cccc1C(=O)NCc1cc(F)cc(F)c1] |
| 306 | 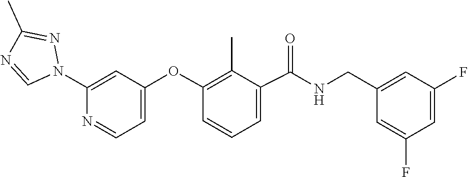 [SMILES: Cc1ncn(-c2cc(Oc3cccc(C(=O)NCc4cc(F)cc(F)c4)c3C)ccn2)n1] |
| 307 | 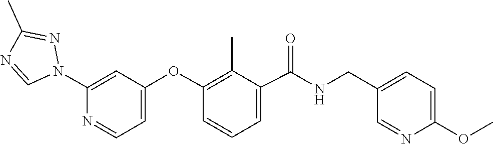 [SMILES: COc1ccc(CNC(=O)c2cccc(Oc3ccnc(-n4cnc(C)n4)c3)c2C)cn1] |
| 308 | 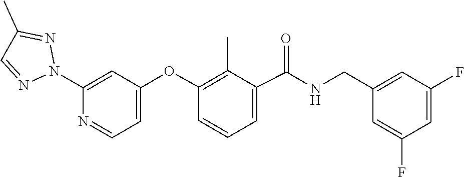 [SMILES: Cc1cnn(-c2cc(Oc3cccc(C(=O)NCc4cc(F)cc(F)c4)c3C)ccn2)n1] |
| 309 | 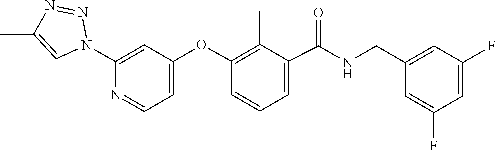 [SMILES: Cc1cn(-c2cc(Oc3cccc(C(=O)NCc4cc(F)cc(F)c4)c3C)ccn2)nn1] |
| 310 | 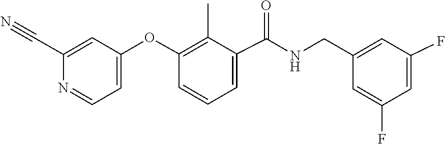 [SMILES: Cc1c(Oc2ccnc(C#N)c2)cccc1C(=O)NCc1cc(F)cc(F)c1] |
| 311 | 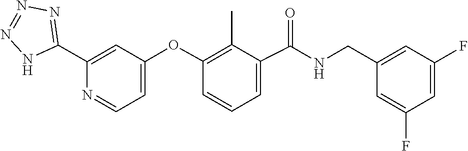 [SMILES: Cc1c(Oc2ccnc(-c3nnn[nH]3)c2)cccc1C(=O)NCc1cc(F)cc(F)c1] |
| 312 | 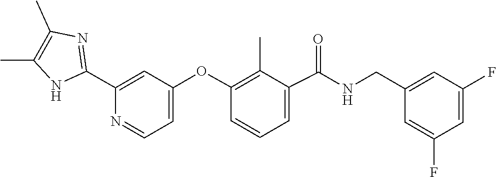 [SMILES: Cc1nc(-c2cc(Oc3cccc(C(=O)NCc4cc(F)cc(F)c4)c3C)ccn2)[nH]c1C] |
| 313 | 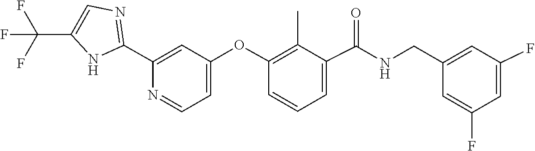 [SMILES: Cc1c(Oc2ccnc(-c3ncc(C(F)(F)F)[nH]3)c2)cccc1C(=O)NCc1cc(F)cc(F)c1] |
| 314 | 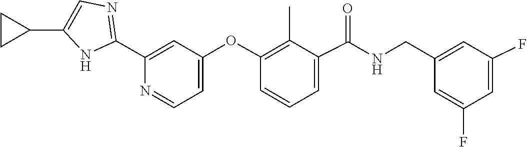 [SMILES: Cc1c(Oc2ccnc(-c3ncc(C4CC4)[nH]3)c2)cccc1C(=O)NCc1cc(F)cc(F)c1] |
| 318 | 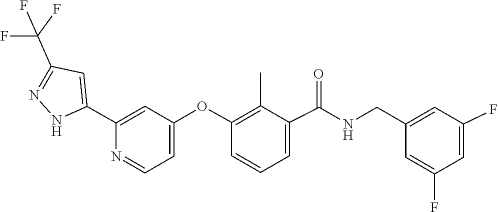 [SMILES: Cc1c(Oc2ccnc(-c3cc(C(F)(F)F)n[nH]3)c2)cccc1C(=O)NCc1cc(F)cc(F)c1] |
| 321 | 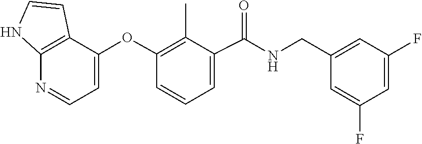 [SMILES: Cc1c(Oc2ccnc3[nH]ccc23)cccc1C(=O)NCc1cc(F)cc(F)c1] |
| 322 | 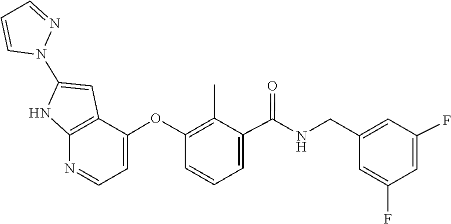 [SMILES: Cc1c(Oc2ccnc3[nH]c(-n4cccn4)cc23)cccc1C(=O)NCc1cc(F)cc(F)c1] |
| 327 | 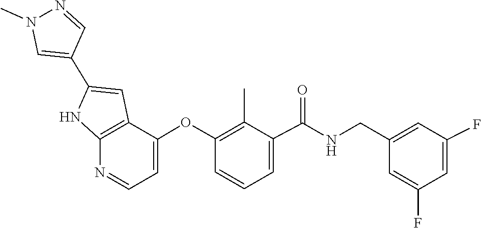 [SMILES: Cc1c(Oc2ccnc3[nH]c(-c4cnn(C)c4)cc23)cccc1C(=O)NCc1cc(F)cc(F)c1] |
| 328 | 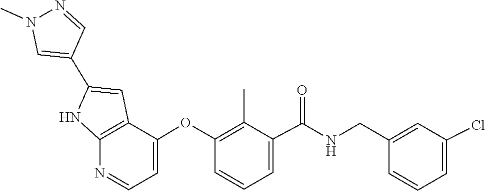 [SMILES: Cc1c(Oc2ccnc3[nH]c(-c4cnn(C)c4)cc23)cccc1C(=O)NCc1cccc(Cl)c1] |
| 329 | 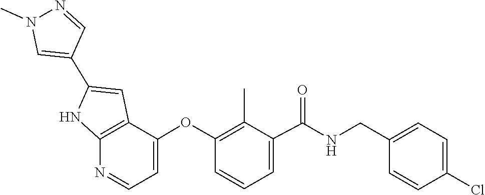 [SMILES: Cc1c(Oc2ccnc3[nH]c(-c4cnn(C)c4)cc23)cccc1C(=O)NCc1ccc(Cl)cc1] |
| 330 | 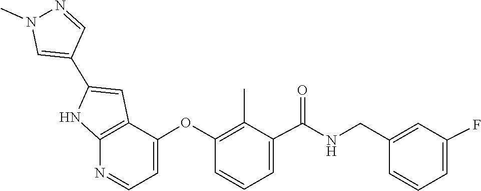 [SMILES: Cc1c(Oc2ccnc3[nH]c(-c4cnn(C)c4)cc23)cccc1C(=O)NCc1cccc(F)c1] |
| 331 | 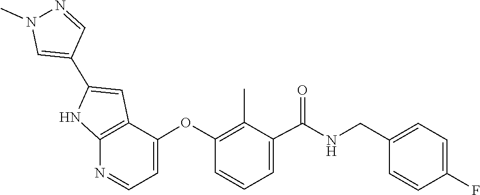 [SMILES: Cc1c(Oc2ccnc3[nH]c(-c4cnn(C)c4)cc23)cccc1C(=O)NCc1ccc(F)cc1] |
| 332 | 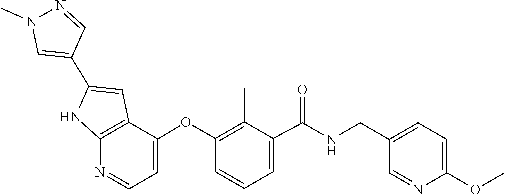 |
| 333 | 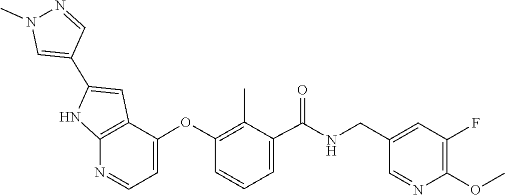 |
| 334 | 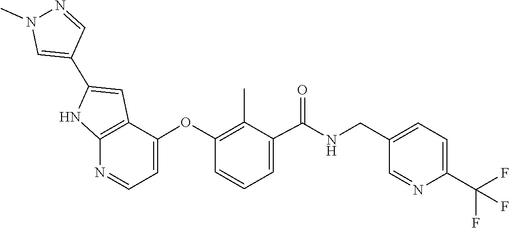 [SMILES: Cc1c(Oc2ccnc3[nH]c(-c4cnn(C)c4)cc23)cccc1C(=O)NCc1ccc(C(F)(F)F)nc1] |
| 335 | 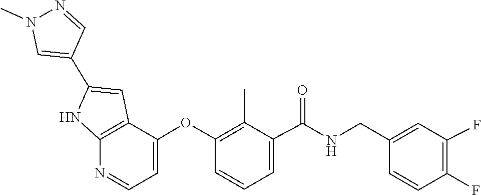 [SMILES: Cc1c(Oc2ccnc3[nH]c(-c4cnn(C)c4)cc23)cccc1C(=O)NCc1ccc(F)c(F)c1] |
| 336 | 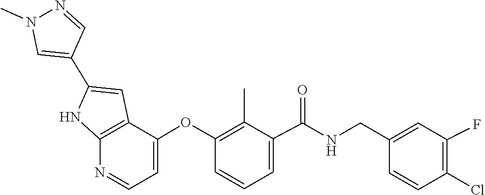 [SMILES: Cc1c(Oc2ccnc3[nH]c(-c4cnn(C)c4)cc23)cccc1C(=O)NCc1ccc(Cl)c(F)c1] |
| 337 | 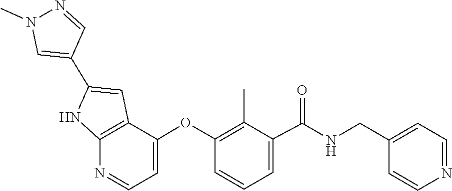 [SMILES: Cc1c(Oc2ccnc3[nH]c(-c4cnn(C)c4)cc23)cccc1C(=O)NCc1ccncc1] |
| 338 | 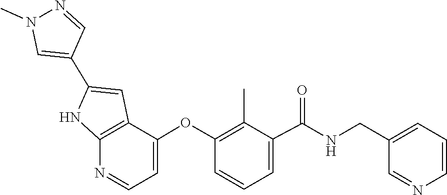 [SMILES: Cc1c(Oc2ccnc3[nH]c(-c4cnn(C)c4)cc23)cccc1C(=O)NCc1cccnc1] |
| 341 | 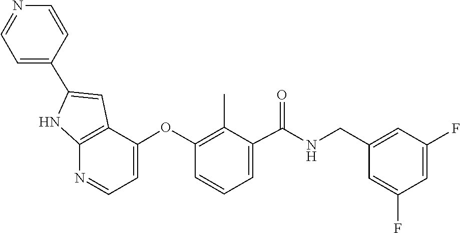 [SMILES: Cc1c(Oc2ccnc3[nH]c(-c4ccncc4)cc23)cccc1C(=O)NCc1cc(F)cc(F)c1] |
| 342 | 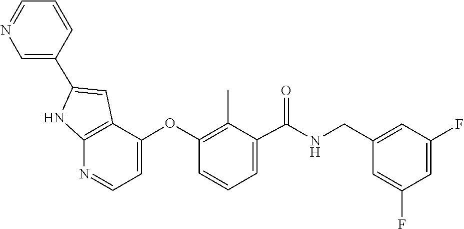 [SMILES: Cc1c(Oc2ccnc3[nH]c(-c4cccnc4)cc23)cccc1C(=O)NCc1cc(F)cc(F)c1] |
| 343 | 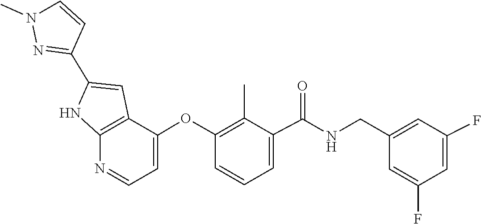 [SMILES: Cc1c(Oc2ccnc3[nH]c(-c4ccn(C)n4)cc23)cccc1C(=O)NCc1cc(F)cc(F)c1] |
| 344 | 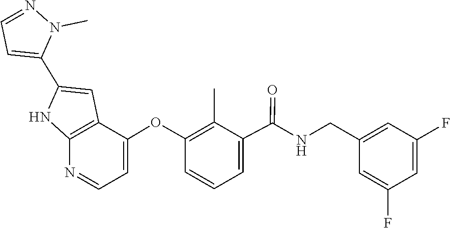 [SMILES: Cc1c(Oc2ccnc3[nH]c(-c4ccnn4C)cc23)cccc1C(=O)NCc1cc(F)cc(F)c1] |
| 347 | 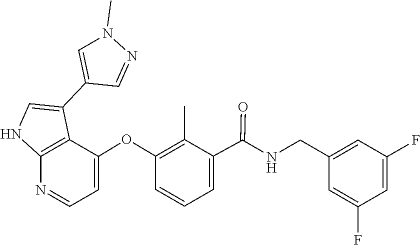 [SMILES: Cc1c(Oc2ccnc3[nH]cc(-c4cnn(C)c4)c23)cccc1C(=O)NCc1cc(F)cc(F)c1] |
| 348 | 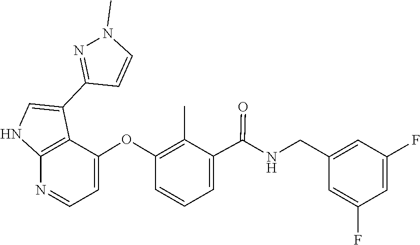 [SMILES: Cc1c(Oc2ccnc3[nH]cc(-c4ccn(C)n4)c23)cccc1C(=O)NCc1cc(F)cc(F)c1] |
| 349 | 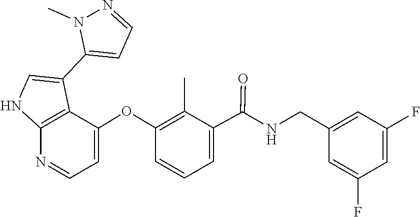 [SMILES: Cc1c(Oc2ccnc3[nH]cc(-c4ccnn4C)c23)cccc1C(=O)NCc1cc(F)cc(F)c1] |
| 350 | 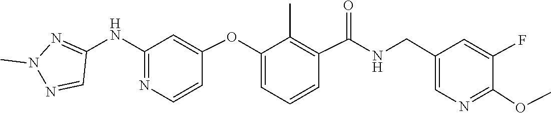 |
| 351 | 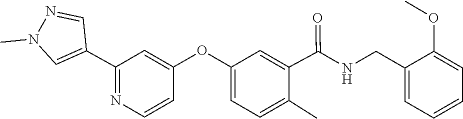 [SMILES: COc1ccccc1CNC(=O)c1cc(Oc2ccnc(-c3cnn(C)c3)c2)ccc1C] |
| 352 | 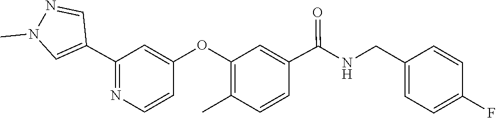 [SMILES: Cc1ccc(C(=O)NCc2ccc(F)cc2)cc1Oc1ccnc(-c2cnn(C)c2)c1] |
| 353 | 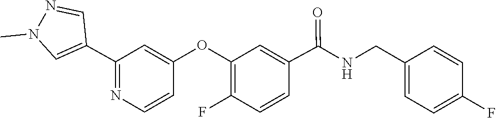 [SMILES: Cn1cc(-c2cc(Oc3cc(C(=O)NCc4ccc(F)cc4)ccc3F)ccn2)cn1] |
| 354 | 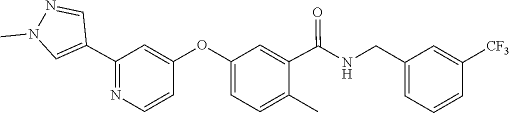 [SMILES: Cc1ccc(Oc2ccnc(-c3cnn(C)c3)c2)cc1C(=O)NCc1cccc(C(F)(F)F)c1] |
| 355 | 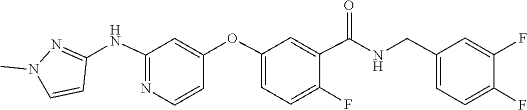 [SMILES: Cn1ccc(Nc2cc(Oc3ccc(F)c(C(=O)NCc4ccc(F)c(F)c4)c3)ccn2)n1] |
| 356 | 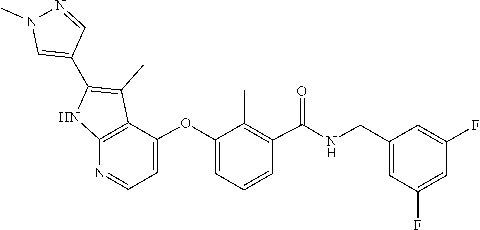 [SMILES: Cc1c(Oc2ccnc3[nH]c(-c4cnn(C)c4)c(C)c23)cccc1C(=O)NCc1cc(F)cc(F)c1] |
| 357 | 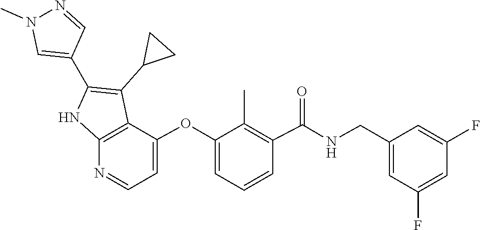 [SMILES: Cc1c(Oc2ccnc3[nH]c(-c4cnn(C)c4)c(C4CC4)c23)cccc1C(=O)NCc1cc(F)cc(F)c1] |
| 358 | 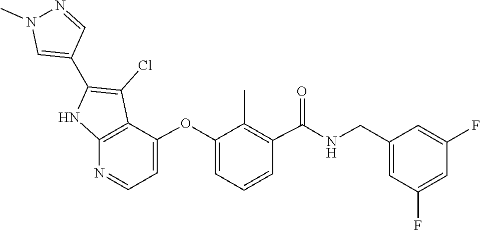 [SMILES: Cc1c(Oc2ccnc3[nH]c(-c4cnn(C)c4)c(Cl)c23)cccc1C(=O)NCc1cc(F)cc(F)c1] |
| 359 | 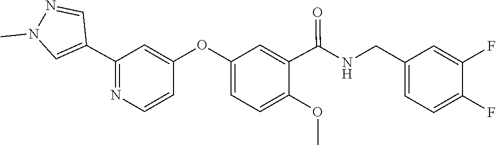 [SMILES: COc1ccc(Oc2ccnc(-c3cnn(C)c3)c2)cc1C(=O)NCc1ccc(F)c(F)c1] |

### Example 24: Cell-Based Assays: Biological Assay Measuring Cell Proliferation in Cell Lines

Compounds were evaluated in different cancer cell lines (Molm-13, M-NFS-60, HL-60 and P815) and in PDGFRα-BaF3 stable cell line. For each, cell proliferation were measured. The protocols of these assays are described below.

MOLM-13: Exponential growing MOLM-13 cells (DSMZ, ACC-554) were seeded at 2.10\hat{ }4 per 200 μl of complete medium. 20 μL of test compound dilution were added to each well and the plates were incubated for 72 h at 37° C., 5% CO₂.

Untreated cells and positive control (0.5% triton X-100, for the last 15 min) served as reference for maximum and minimum viability. At the end of incubation 100 μl of supernatant were removed and replaced by 10 μl of WST-1 solution (Cell Proliferation Reagent WST-1, Roche Applied Science). After 3 h incubation at 37° C., 5% CO₂, optical densities were measured at 450 nm and 620 nm for the background on microplate reader (Envision 2105, Perkinelmer).

M-NFS-60: Exponential growing M-NFS-60 cells (ATCC, CRL-1838) were seeded at 10\hat{ }4 per 200 μl of complete medium with beta-mercaptoethanol and M-CSF (62 ng/mL) or IL34 (500 ng/mL). Twenty μL of test compound dilution were added to each well and the plates were incubated for 72 h at 37° C., 5% CO₂. Untreated cells and positive control (0.5% triton X-100, for the last 15 min) served as reference for maximum and minimum viability. At the end of incubation 100 μl of supernatant were removed and replaced by 10 μl of WST-1 solution (Cell Proliferation Reagent WST-1, Roche Applied Science). After 3 h incubation at 37° C., 5% CO₂, optical densities were measured at 450 nm and 620 nm for the background on microplate reader (Envision 2105, Perkinelmer).

HL-60: Exponential growing HL-60 cells (DSMZ, ACC-3) were seeded at 2.10\hat{ }4 per 200 μl of complete RPMI medium. 20 μL of test compound dilution were added to each well and the plates were incubated for 72 h at 37° C., 5% CO₂. Untreated cells and positive control (0.5% triton X-100, for the last 15 min) served as reference for maximum and minimum viability. At the end of incubation 100 μl of supernatant were removed and replaced by 10 μl of WST-1 solution (Cell Proliferation Reagent WST-1, Roche Applied Science). After 3 h incubation at 37° C., 5% CO₂, optical densities were measured at 450 nm and 620 nm for the background on microplate reader (Envision 2105, Perkinelmer).

IC50 were measured and some biological results of these assays are presented in the following table.

P-815: Exponential growing P-815 cells (DSMZ, ACC-1) were seeded at 2.10\hat{ }4 per 200 μl of complete RPMI medium. Twenty μL of test compound dilution were added to each well and the plates were incubated for 72 h at 37° C., 5% CO₂. Untreated cells and positive control (0.5% triton X-100, for the last 15 min) served as reference for maximum and minimum viability. At the end of incubation 100 μl of supernatant were removed and replaced by 10 μl of WST-1 solution (Cell Proliferation Reagent WST-1, Roche Applied Science). After 3 h incubation at 37° C., 5% CO₂, optical densities were measured at 450 nm and 620 nm for the background on microplate reader (Envision 2105, Perkinelmer).

BaF3-PDGFRα: Exponential growing BaF3 cells stably transfected with a plasmid encoding the fusion gene GFP-ETV6-PDGFRA (ABMGood, T3082) were seeded at 5.10\hat{ }3 per 200 μl of complete RPMI medium. Twenty μL of test compound dilution were added to each well and the plates were incubated for 72 h at 37° C., 5% CO₂. Untreated cells and positive control (0.5% triton X-100, for the last 15 min) served as reference for maximum and minimum viability. At the end of incubation 100 μl of supernatant were removed and replaced by 10 μl of WST-1 solution (Cell Proliferation Reagent WST-1, Roche Applied Science). After 3 h incubation at 37° C., 5% CO₂, optical densities were measured at 450 nm and 620 nm for the background on microplate reader (Envision 2105, Perkinelmer).

IC50 were measured and some biological results of these assays are presented in the following table.

NB IC50 are reported as follows

### Example 25: Cell-Based Assays: Biological Assay Measuring Cell Proliferation in Non-Cancer Cell Lines

CSF1R receptor has been expressed in HEK cell lines following the protocols below.

HEK-CSF1R-STAT5-Luc: Exponential growing HEK293T cells (ATCC® CRL-3216TM), ectopically expressing human CSF1R receptor (Origene) and five copies of a STAT5 response element (STAT5 RE, promega) that drives transcription of the luciferase reporter were seeded at 5.10\hat{ }3 per 20 μl of complete DMEM medium. The next day, 2.25 μL of test compound dilution were added to each well and stimulated with 600 ng/ml of M-CSF. The plates were incubated for 24 h at 37° C., 5% CO₂. Unstimulated and stimulated cells served as reference for maximum and minimum induction. At the end of incubation 25 μl of Steady-Glo® Luciferase Assay System (Promega) were added after 5 min of lysis, luminescence was measured on microplate reader (Envision 2105, Perkinelmer).

HEK-CSF1R-WST-1: Exponential growing HEK293T cells (ATCC® CRL-3216™), were seeded at 5.10\hat{ }3 per 200 μl of complete DMEM medium. The next day, twenty μL of test compound dilution were added to each well and the plates were incubated for 72 h at 37° C., 5% CO₂. Untreated cells and positive control (0.5% triton X-100, for the last 15 min) served as reference for maximum and minimum viability. At the end of incubation 100 μl of supernatant were removed and replaced by 10 μl of WST-1 solution (Cell Proliferation Reagent WST-1, Roche Applied Science). After 3 h incubation at 37° C., 5% CO₂, optical densities were measured at 450 nm and 620 nm for the background, on microplate reader (Envision 2105, Perkinelmer).

IC50 were measured and some biological results of these assays are presented in the following table.

NB IC50 are reported as follows

1. A compound (C) or an N-oxide, pharmaceutically acceptable salt, pharmaceutically acceptable solvate, or stereoisomer thereof, wherein said compound (C) is chosen among those of formulae (I) to (VII) 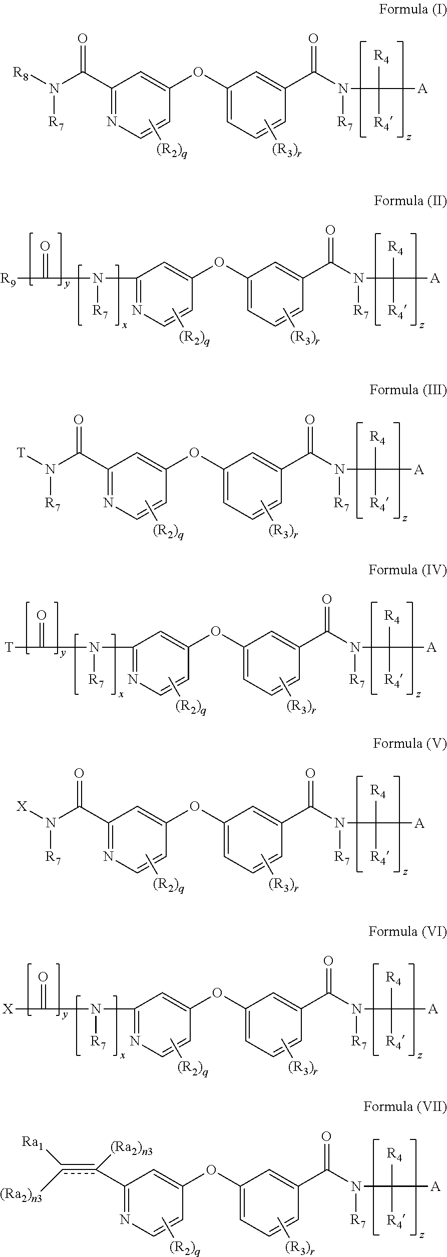
wherein:
each of A is independently selected from the group consisting of cycloalkyl, heterocyclyl, aryl, heteroaryl, and aralkyl, wherein said cycloalkyl, heterocyclyl, aryl, heteroaryl, and aralkyl are optionally substituted with one or more substituents independently selected from the group consisting of halo, NO₂, C₁₋₆ alkyl, C₂₋₄ alkenyl, C₂₋₄ alkynyl, cycloalkyl, heterocyclyl, aryl, heteroaryl, CF₃, CN, OR₁₁, SR₁₁, N(R₁₁)₂, OC(R₁₁)₂O, OC(R₁₁)₂C(R₁₁)₂O, S(O)R₁₂, SO₂R₁₂, SO₂N(R₁₁)₂, S(O)₃R₁₁, P(═O)(OR₁₁)₂, P(═O)(R₁₁)₂ NR₁₁COR₁₂, COR₁₁, C(O)OR₁₁, CON(R₁₁)₂, OC(O)R₁₁, and OCON(R₁₁)₂, and each optional alkyl, alkenyl, alkynyl, cycloalkyl, aryl, heterocyclyl, and heteroaryl substituents is further optionally substituted with halo, NO₂, C₁₋₆ alkyl, cycloalkyl, aryl, CF₃, N(R₁₁)₂, COR₁₁, CON(R₁₁)₂, OC(O)R₁₁, CN, or OR₁₁; and wherein each of R₁₁ and R₁₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, C₁₋₆ alkyl or aryl or heteroaryl amide, OR₃₁ or N(R₃₂)₂, wherein each of R₃₁ and R₃₂, independently from each other and at each occurrence, is selected from the group consisting of hydrogen and C₁₋₄ alkyl.
each of R₄ and R′₄, independently from each other and at each occurrence, are selected from hydrogen or C₁₋₆ alkyl, and z is an integer in a range from 0 to 2; with the proviso that when z=0, then A and R₇ may form together a saturated or unsaturated cyclic moiety;
each of R₇, independently from each other and at each occurrence is selected from hydrogen, C₁₋₆ alkyl, cycloalkyl, wherein said alkyl and cycloalkyl are optionally substituted by a halogen atom, CF₃, N(R₁₁)₂, CN, or OR₁₁; and wherein each of R₁₁, independently from each other and at each occurrence, is selected from the group consisting of hydrogen, C₁₋₆ alkyl, and CF₃;
each of R₃, independently from each other and at each occurrence, is selected from the group consisting of hydrogen, halo, C₁₋₆ alkyl, C₂₋₆ alkenyl, C₂₋₆ alkynyl, cycloalkyl, heterocyclyl, aryl, heteroaryl, aralkyl, CF₃, CN, OR₂₁, SR₂₁, N(R₂₁)₂, NC(O)R₂₁, NCON(R₂₁)₂, COR₂₁, C(O)OR₂₁, CON(R₂₁)₂, OC(O)R₂₁, OCON(R₂₁)₂, OC(R₂₁)₂O, and OC(R₂₁)₂C(R₂₂)₂O, wherein said alkyl, alkenyl, alkynyl, cycloalkyl, heterocyclyl, aryl, heteroaryl and aralkyl are optionally substituted with one or more substituents selected from halo, C₁₋₆ alkyl, CF₃, N(R₂₁)₂, CN, or OR₂₁; and wherein each of R₂₁ and R₂₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, and aralkyl, and wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, aryl, OR₃₁ or N(R₃₂)₂, wherein each of R₃₁ and R₃₂, independently from each other and at each occurrence, is selected from the group consisting of hydrogen and C₁₋₄ alkyl; each of r is an integer in the range from 0 to 3; with the proviso that when R₃═NR₂₁, and R₇═H, then R₃ and NR₇ may form together a saturated or unsaturated cyclic moiety;
each of R₂, independently from each other and at each occurrence, is selected from the group consisting of hydrogen, halo, C₁₋₆ alkyl, C₂₋₆ alkenyl, C₂₋₆ alkynyl, cycloalkyl, heterocyclyl, aryl, and heteroaryl, CF₃, CN, NO₂, OR₂₁, SR₂₁, N(R₂₁)₂, COR₂₁, C(O)OR₂₁, CON(R₂₁)₂, OC(O)R₂₁, OCON(R₂₁)₂, NC(O)R₂₁, NCON(R₂₁)₂, OC(R₂₁)₂O and OC(R₂₁)₂C(R₂₂)₂O, wherein said alkyl, alkenyl, alkynyl, cycloalkyl, heterocyclyl, aryl, and heteroaryl are optionally substituted with one or more substituents selected from halo, C₁₋₆ alkyl, C₂₋₆ alkenyl, C₂₋₆ alkynyl, cycloalkyl, heterocyclyl, CF₃, COR₂₁, CON(R₂₁)₂, C(O)OR₂₁, N(R₂₁)₂, CN, or OR₂₁, and each optional alkyl, alkenyl, alkynyl, cycloalkyl, heterocyclyl, aryl or heteroaryl substituent is further optionally substituted with heterocyclyl, N(R₁₁)₂, or OR₁₁; and wherein each of R₂₁ and R₂₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, and aralkyl, and wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, aryl, OR₃₁ or N(R₃₂)₂, wherein each of R₃₁ and R₃₂, independently from each other and at each occurrence, is selected from the group consisting of hydrogen and C₁₋₄ alkyl; each of q is an integer in the range from 0 to 2;
each of x and y are independently integers equal to 0 or 1;
R₈ is independently selected from the group consisting of C₆₋₁₂ alkyl, C₂₋₆ alkenyl, C₂₋₆ alkynyl, cycloalkyl and heterocyclyl, wherein said alkyl, alkenyl, alkynyl, cycloalkyl, and heterocyclyl are optionally substituted by a halogen atom, an aryl group, an aralkyl group, CF₃, N(R₁₁)₂, CN, or OR₁₁; and wherein each of R₁₁, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, aryl, heteroaryl, and aralkyl substituents are optionally substituted with halo, C₁₋₆ alkyl, cycloalkyl, heterocyclyl, alkyl or aryl or heteroaryl amide, OR₃₁ or N(R₃₂)₂, wherein each of R₃₁ and R₃₂, independently from each other and at each occurrence, is selected from the group consisting of hydrogen and C₁₋₄ alkyl;
R₉ is selected from the group consisting of hydrogen, C₁₋₆ alkyl, C₂₋₆ alkenyl, C₂₋₆ alkynyl, cycloalkyl, heterocyclyl, N(R₁₁)₂ and CN, wherein said alkyl, alkenyl, alkynyl, cycloalkyl, and heterocyclyl are optionally substituted by a halogen atom, an aryl group, an aralkyl group, an heterocyclyl group, CF₃, N(R₁₁)₂, CN, or OR₁₁; and wherein each of R₁₁, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, heteroaryl optionally substituted with a C₁₋₄ alkyl, aryl, heteroaryl, and aralkyl substituents are optionally substituted with halo, C₁₋₆ alkyl, cycloalkyl, heterocyclyl, alkyl or aryl or heteroaryl amide, OR₃₁ or N(R₃₂)₂, wherein each of R₃₁ and R₃₂, independently from each other and at each occurrence, is selected from the group consisting of hydrogen and C₁₋₄ alkyl, with the proviso that if x=1 and y=0, R₉ is different from heterocyclyl, and from C₁₋₆ alkyl wherein said alkyl is optionally substituted with heterocyclyl; and with the proviso that if x=0 and γ=0, R₉ is different from hydrogen, and C₁₋₆ alkyl, wherein said alkyl is optionally substituted with heterocyclyl and N(R₁₁)₂; with the proviso that when x=0 and y=0, R₉ and R₂ may form together a saturated or an unsaturated cyclic moiety; with the proviso that when x=0 and γ=0 and when R₉ and R₂ form together a saturated or an unsaturated cyclic moiety, R₉ is NR₁₁; with the proviso that when x=1 and y=1, R₉ is different from N(R₁₁)₂; and with the proviso that when x=0, y=0 and z=0, R₉ is different from pyrrole;
each of T is independently the unsaturated cyclic moiety of formula (T-a) herein below: 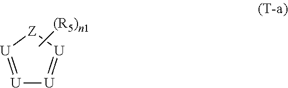
wherein:
each of U, independently from each other and at each occurrence, is selected from the group consisting of C, C-halo, C—R, and N; wherein R is selected from hydrogen, OR₁₁, N(R₁₁)₂, a C₁₋₆ alkyl or a cycloalkyl which are optionally substituted by a halogen atom, an aryl group or an aralkyl group, wherein each of R₁₁, independently from each other and at each occurrence, is selected from the group consisting of hydrogen or C₁₋₄ alkyl; with the proviso that at least one U is different from N;
each of Z, independently from each other and at each occurrence is selected from C(R)₂, O, S and NR₇, wherein R, independently from each other and at each occurrence is selected from hydrogen or an C₁₋₆ alkyl which is optionally substituted by a halogen atom, an aryl group or an aralkyl group, wherein R₇ is selected from the group consisting of hydrogen, C₁₋₆ alkyl, C₁₋₆ alkenyl, cycloalkyl, heterocyclyl, aryl, aralkyl and CF₃;
each of R₅, independently from each other and at each occurrence is selected from the group consisting of C₁₋₆ alkyl, C₂₋₆ alkenyl, C₂₋₆ alkynyl, cycloalkyl, heterocyclyl, aryl, heteroaryl, aralkyl, halo, CF₃, OR₁₁, SR₁₁, N(R₁₁)₂, COOR₁₁, CO(R₁₁)₂, CON(R₁₁)₂, and each optional alkyl, alkenyl, alkynyl, cycloalkyl, aryl, heterocyclyl, heteroaryl, aralkyl substituent is further optionally substituted with halo, C₁₋₆ alkyl, cycloalkyl, aryl, heterocyclyl, N(R₁₁)₂, CN, OR₁₁, C(═O)OR₁₁, P(═O)(OR₁₁)₂, P(═O)(R₁₁)₂, CN or CF₃ and wherein each of R₁₁, independently from each other and at each occurrence, is selected from the group consisting of hydrogen, C₁₋₆ alkyl, cycloalkyl, and heterocyclyl; each of n1 is an integer in the range from 0 to 2;
each of X is independently the moiety of formula (X-a) herein below: 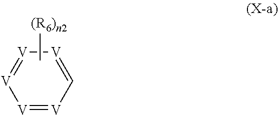
wherein:
each of V, independently from each other and at each occurrence, is selected from the group consisting of C, C-halo, C—R, and N; wherein R is selected from hydrogen, OR₁₁, N(R₁₁)₂, a C₁₋₆ alkyl or a cycloalkyl which are optionally substituted by a halogen atom, an aryl group or an aralkyl group, wherein each of R₁₁, independently from each other and at each occurrence, is selected from hydrogen or C₁₋₄ alkyl;
each of R₆, independently from each other and at each occurrence is selected from the group consisting of C₁₋₆ alkyl, C₂₋₆ alkenyl, C₂₋₆ alkynyl, cycloalkyl, heterocyclyl, aryl, heteroaryl, aralkyl, halo, CF₃, OR₁₁, SR₁₁, N(R₁₁)₂, COOR₁₁, CO(R₁₁)₂, CON(R₁₁)₂, and each optional alkyl, alkenyl, alkynyl, cycloalkyl, aryl, heterocyclyl, heteroaryl, aralkyl substituent is further optionally substituted with halo, C₁₋₆ alkyl, cycloalkyl, aryl, heterocyclyl, N(R₁₁)₂, CN, OR₁₁, C(═O)OR₁₁, P(═O)(OR₁₁)₂, P(═O)(R₁₁)₂, CN or CF₃ and wherein each of R₁₁, independently from each other and at each occurrence, is selected from the group consisting of hydrogen, C₁₋₆ alkyl, cycloalkyl, and heterocyclyl; each of n2 is an integer in the range from 0 to 4;
the dash bond represents an optional triple bond;
Rₐ₁ is independently selected from the group consisting of hydrogen, C₁₋₆ alkyl, cycloalkyl, heterocyclyl, aryl, heteroaryl, aralkyl, CF₃, CN, OR₁₁, SR₁₁, N(R₁₁)₂, COR₁₁, C(O)OR₁₁, wherein said alkyl cycloalkyl, heterocyclyl, aryl, heteroaryl and aralkyl are optionally substituted by halo, NO₂, C₁₋₆ alkyl, C₂₋₆ alkenyl, C₂₋₆ alkynyl, cycloalkyl, heterocyclyl, phenyl, heteroaryl, CF₃, CN, OR₁₁, SR₁₁, N(R₁₁)₂, COR₁₁, and C(O)OR₁₁, and each optional alkyl, alkenyl, cycloalkyl, phenyl, heterocyclyl, heteroaryl substituent is further optionally substituted with halo, NO₂, C₁₋₆ alkyl, cycloalkyl, phenyl, N(R₁₁)₂, CN, or OR₁₁; and wherein each of R₁₁ is selected from the group consisting of hydrogen, C₁₋₆ alkyl, cycloalkyl, heterocyclyl, phenyl, heteroaryl, and aralkyl, wherein said alkyl, cycloalkyl, heterocyclyl, phenyl, heteroaryl, and aralkyl substituents are optionally substituted with halo, C₁₋₆ alkyl, cycloalkyl or heterocyclyl; and
each of Rₐ₂, independently from each other and at each occurrence, is selected from the group consisting of hydrogen, C₁₋₆ alkyl, cycloalkyl, heterocyclyl, aryl, heteroaryl, aralkyl, CF₃, CN, OR₁₁, SR₁₁, N(R₁₁)₂, COR₁₁, C(O)OR₁₁, wherein said alkyl, alkenyl, alkynyl, cycloalkyl, heterocyclyl, aryl, heteroaryl and aralkyl are optionally substituted by halo, NO₂, C₁₋₆ alkyl, C₂₋₆ alkenyl, C₂₋₆ alkynyl, cycloalkyl, heterocyclyl, phenyl, heteroaryl, CF₃, CN, OR₁₁, SR₁₁, N(R₁₁)₂, COR₁₁, and C(O)OR₁₁, and each optional alkyl, alkenyl, cycloalkyl, phenyl, heterocyclyl, heteroaryl substituent is further optionally substituted with halo, NO₂, C₁₋₆ alkyl, cycloalkyl, phenyl, N(R₁₁)₂, CN, or OR₁₁; and wherein each of Rᵤ is selected from the group consisting of hydrogen, C₁₋₆ alkyl, cycloalkyl, heterocyclyl, phenyl, heteroaryl, and aralkyl, wherein said alkyl, cycloalkyl, heterocyclyl, phenyl, heteroaryl, and aralkyl substituents are optionally substituted with halo, C₁₋₆ alkyl, cycloalkyl or heterocyclyl; and wherein n3 is an integer equal to 0 or 1; with the proviso that when the dash bond represents a triple bond, n3 is 0,
wherein said cycloalkyl is a monocyclic, bicyclic or tricyclic ring system of 3-6 ring members per ring; said heterocyclyl is a saturated, partially saturated or completely saturated monocycle, bicycle or tricycle containing 3 to 12 carbon atoms and 1 or 2 heteroatoms independently selected from O or N; said aryl is phenyl, naphthyl or anthracenyl optionally carbocyclic fused with a cycloalkyl or heterocyclyl of 5-7 ring members; said heteroaryl is a monocyclic ring structure containing 5 or 6 ring atoms, or a bicyclic aromatic group having 8 to 10 atoms, containing 1-3 heteroatoms independently selected from O or N.

2. The compound (C) according to claim 1, or the N-oxide, pharmaceutically acceptable salt, pharmaceutically acceptable solvate, or stereoisomer thereof, wherein x and y are as defined as in claim 1 and wherein:
each of A is independently selected from the group consisting of cycloalkyl, heterocyclyl, aryl, heteroaryl, and aralkyl, wherein said cycloalkyl, heterocyclyl, aryl, heteroaryl, and aralkyl are optionally substituted with one or more substituents independently selected from the group consisting of halo, NO₂, C₁₋₆ alkyl, C₂₋₄ alkenyl, C₂₋₄ alkynyl, cycloalkyl, heterocyclyl, aryl, heteroaryl, CF₃, CN, OR₁₁, SR₁₁, N(R₁₁)₂, OC(R₁₁)₂O, OC(R₁₁)₂C(R₁₁)₂O, P(═O)(OR₁₁)₂, P(═O)(R₁₁)₂ NR₁₁COR₁₂, COR₁₁, C(O)OR₁₁, CON(R₁₁)₂, OC(O)R₁₁, and OCON(R₁₁)₂, and each optional alkyl, alkenyl, alkynyl, cycloalkyl, aryl, heterocyclyl, heteroaryl substituent is further optionally substituted with halo, NO₂, C₁₋₆ alkyl, cycloalkyl, aryl, CF₃, N(R₁₁)₂, CN, or OR₁₁; and wherein each of R₁₁ and R₁₂, independently from each other and at each occurrence, is selected from the group consisting of hydrogen, C₁₋₆ alkyl, C₂₋₆ alkenyl, C₂₋₆ alkynyl, cycloalkyl, heterocyclyl, aryl, heteroaryl, aralkyl and CF₃, wherein said alkyl, alkenyl, alkynyl, cycloalkyl, heterocyclyl, aryl, heteroaryl, and aralkyl substituents are optionally substituted with halo, C₁₋₆ alkyl, cycloalkyl, or heterocyclyl;
each of R₄ and R′₄, independently from each other and at each occurrence, are selected from hydrogen or C₁₋₆ alkyl; and wherein z is an integer equal to 1;
each of R₇, independently from each other and at each occurrence is hydrogen or C₁₋₄ alkyl such as methyl, ethyl, propyl, isopropyl, butyl, tert-butyl, isobutyl and the like;
each of R₃, independently from each other and at each occurrence, is selected from the group consisting of hydrogen, halo, C₁₋₆ alkyl, cycloalkyl, heterocyclyl, CF₃, CN, OR₂₁, and N(R₂₁)₂, wherein said alkyl, cycloalkyl and heterocyclyl, are optionally substituted with one or more substituents selected from halo, C₁₋₆ alkyl, CF₃, N(R₂₁)₂, CN, or OR₂₁; and wherein each of R₂₁, independently from each other and at each occurrence, is selected from the group consisting of hydrogen, C₁₋₆ alkyl, C₃₋₆ cycloalkyl, heterocyclyl, aryl, heteroaryl, and aralkyl, and wherein said alkyl, cycloalkyl, heterocyclyl, aryl, heteroaryl, and aralkyl substituents are optionally substituted with halo, C₁₋₆ alkyl, cycloalkyl, heterocyclyl, or aryl; each of r is an integer equal to 0 or 1;
each of R₂, independently from each other and at each occurrence, is selected from the group consisting of hydrogen, halo, C₁₋₆ alkyl, cycloalkyl, heterocyclyl, aryl, and heteroaryl, CN, OR₂₁, and N(R₂₁)₂, wherein said alkyl, cycloalkyl, heterocyclyl, aryl, and heteroaryl are optionally substituted with one or more substituents selected from halo, C₁₋₆ alkyl, cycloalkyl, N(R₂₁)₂, CN, or OR₂₁; wherein R₂₁, independently from each other and at each occurrence, is selected from the group consisting of hydrogen, C₁₋₆ alkyl, cycloalkyl, heterocyclyl, aryl, heteroaryl, and aralkyl.
R₈ is selected from the group consisting of C₆₋₁₂ alkyl, cycloalkyl and heterocyclyl, wherein said alkyl, cycloalkyl, and heterocyclyl are optionally substituted by a halogen atom, CF₃, N(R₁₁)₂, CN, or OR₁₁; and wherein each of R₁₁, independently from each other and at each occurrence, is selected from the group consisting of hydrogen, and C₁₋₆ alkyl;
R₉ is selected from the group consisting of hydrogen, C₁₋₆ alkyl, cycloalkyl, N(R₁₁)₂, and CN, wherein said alkyl, and cycloalkyl, are optionally substituted by a halogen atom, CF₃, CN, or OR₁₁; and wherein each of R₁₁, independently from each other and at each occurrence, is selected from the group consisting of hydrogen, C₁₋₆ alkyl, C₂₋₆ alkenyl, and CF₃, wherein said alkyl, and alkenyl substituents are optionally substituted with an heteroaryl group optionally substituted with a C₁₋₄ alkyl with the proviso that if x=0 and y=0, R₉ is different from hydrogen and C₁₋₆ alkyl, wherein said alkyl is optionally substituted with heterocyclyl and N(R₁₁)₂ and with the proviso that when x=0 and y=0, R₉ and R₂ may form together a saturated or an unsaturated cyclic moiety; with the proviso that when x=0 and γ=0 and when R₉ and R₂ form together a saturated or an unsaturated cyclic moiety, R₉ is NR₁₁; with the proviso that when x=1 and y=1, R₉ is different from N(R₁₁)₂; and with the proviso that when x=0, y=0 and z=0, R₉ is different from pyrrole;
each of T is independently the moiety of formula (T-a) herein below: 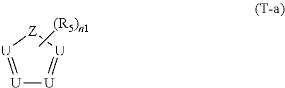
wherein:
each of U is selected independently from each other and at each occurrence, from C, C-halo, C—R, or N; wherein R is hydrogen or C₁₋₄ alkyl with the proviso that at least one U is different from N. More preferably, each of U is selected, independently from each other and at each occurrence, from C, C—R or N; wherein R is hydrogen or C₁₋₄ alkyl with the proviso that at least one U is different from N;
each of Z, is, independently from each other and at each occurrence, preferably selected from the group consisting of CH₂, and O, S and NR₇ wherein R₇ is an hydrogen, or a C₁₋₄ alkyl such as methyl, ethyl, propyl, isopropyl, butyl, tert-butyl, and isobutyl;
each of R₅, independently from each other and at each occurrence is preferably selected from the group consisting of C₁₋₆ alkyl, cycloalkyl, heterocyclyl, aryl, heteroaryl, aralkyl, halo, CF₃, OR₁₁, N(R₁₁)₂, COOR₁₁, CO(R₁₁)₂, CON(R₁₁)₂, and each optional alkyl, cycloalkyl, aryl, heterocyclyl, heteroaryl, aralkyl substituent is further optionally substituted with halo, C₁₋₆ alkyl, cycloalkyl, aryl, heterocyclyl, N(R₁₁)₂, CN, OR₁₁, C(═O)OR₁₁, P(═O)(OR₁₁)₂, P(═O)(R₁₁)₂, CN or CF₃ and wherein each of R₁₁, independently from each other and at each occurrence, is selected from the group consisting of hydrogen, C₁₋₆ alkyl, cycloalkyl, and heterocyclyl; each of n1 is an integer equal to 1 or 2;
each of X is independently the moiety of formula (X-a) herein below: 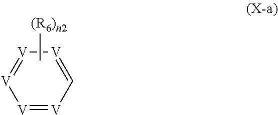
wherein:
each of V, independently from each other and at each occurrence, is selected from the group consisting of C, C-halo, C—R, and N; wherein R is hydrogen or C₁₋₄ alkyl;
each of R₆, independently from each other and at each occurrence is preferably selected from the group consisting of C₁₋₆ alkyl, cycloalkyl, heterocyclyl, aryl, heteroaryl, aralkyl, halo, CF₃, OR₁₁, N(R₁₁)₂, COOR₁₁, CO(R₁₁)₂, CON(R₁₁)₂, and each optional alkyl, cycloalkyl, aryl, heterocyclyl, heteroaryl, aralkyl substituent is further optionally substituted with halo, C₁₋₆ alkyl, cycloalkyl, aryl, heterocyclyl, N(R₁₁)₂, CN, OR₁₁, C(═O)OR₁₁, P(═O)(OR₁₁)₂, P(═O)(R₁₁)₂, CN or CF₃ and wherein each of R₁₁, independently from each other and at each occurrence, is selected from the group consisting of hydrogen, C₁₋₆ alkyl, cycloalkyl, and heterocyclyl; and wherein n2 is an integer equal to 0, 1 or 2;
the dash bond represents an optional triple bond;
Rₐ₁ is independently selected from the group consisting of C₁₋₄ alkyl, cycloalkyl, heterocyclyl, aryl, heteroaryl, aralkyl, wherein said alkyl cycloalkyl, heterocyclyl, aryl, heteroaryl and aralkyl are optionally substituted by halo, NO₂, C₁₋₄ alkyl, cycloalkyl, heterocyclyl, phenyl, heteroaryl, CF₃, CN, OR₁₁, N(R₁₁)₂, and wherein each of R₁₁ is selected from the group consisting of hydrogen, or C₁₋₄ alkyl; and
each of Rₐ₂ is independently selected from the group consisting of C₁₋₄ alkyl, cycloalkyl, heterocyclyl, aryl, heteroaryl, aralkyl, wherein said alkyl cycloalkyl, heterocyclyl, aryl, heteroaryl and aralkyl are optionally substituted by halo, NO₂, C₁₋₄ alkyl, cycloalkyl, heterocyclyl, phenyl, heteroaryl, CF₃, CN, OR₁₁, N(R₁₁)₂, and wherein each of R₁₁ is selected from the group consisting of hydrogen, or C₁₋₄ alkyl,
wherein said cycloalkyl is a monocyclic, bicyclic or tricyclic ring system of 3-6 ring members per ring; said heterocyclyl is a saturated, partially saturated or completely saturated monocycle, bicycle or tricycle containing 3 to 12 carbon atoms and 1 or 2 heteroatoms independently selected from O or N; said aryl is phenyl, naphthyl or anthracenyl optionally carbocyclic fused with a cycloalkyl or heterocyclyl of 5-7 ring members; said heteroaryl is a monocyclic ring structure containing 5 or 6 ring atoms, or a bicyclic aromatic group having 8 to 10 atoms, containing 1-3 heteroatoms independently selected from O or N.

3. The compound (C) of formulae (II) or (III), according to claim 1, or the N-oxide, pharmaceutically acceptable salt, pharmaceutically acceptable solvate, or stereoisomer thereof, wherein said compound is of formula (II-a) or (II-b) [compounds (C) of class (II) hereinafter] or of formula (III-a) [compounds (C) of class (III) hereinafter]: 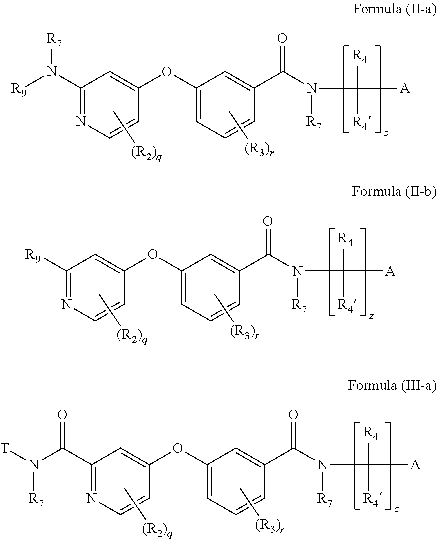
wherein A, R₄, R₄′, z, R₇, R₃, r, R₂, q, R₉ and T are as defined as in claim 1.

4. The compound (C) of formula (IV) or formula (VI), according to claim 1, or the N-oxide, pharmaceutically acceptable salt, pharmaceutically acceptable solvate, or stereoisomer thereof, wherein said compound is of formula (IV-a) to (IV-c) [compounds (C) of class (IV) hereinafter] or of formula (VI-a) to (VI-c) [compounds (C) of class (VI) hereinafter]: 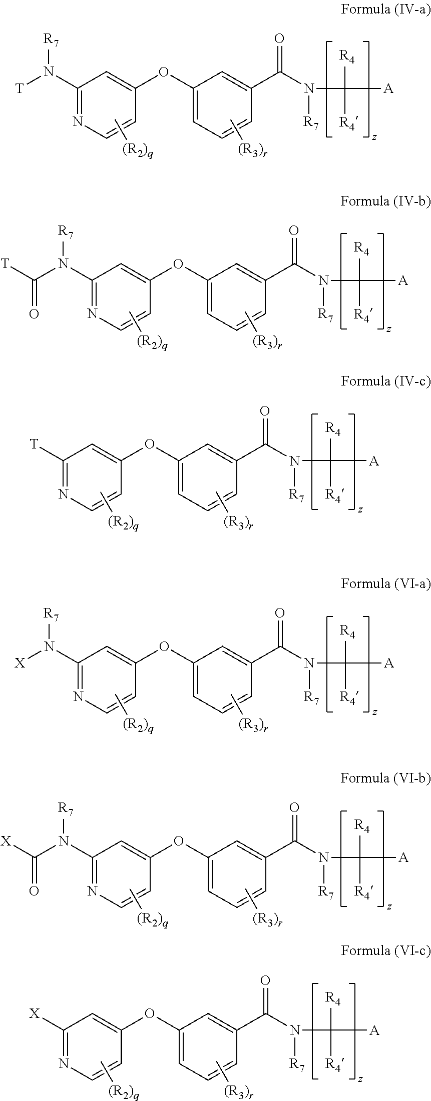
wherein A, R₄, R₄′, z, R₇, R₃, r, R₂, q, T, and X are as defined as in claim 1.

5. The compound (C) of formula (VII), according to claim 1, or the N-oxide, pharmaceutically acceptable salt, pharmaceutically acceptable solvate, or stereoisomer thereof, wherein said compound is of formula (VII-a) or (VII-b) [compounds (C) of class (VII) hereinafter]: 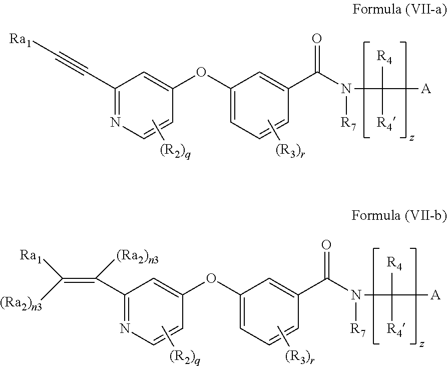
wherein A, R₄, R₄′, z, R₇, R₃, r, R₂, q, Rₐ₁, Rₐ₂ and n3 are as defined as in claim 1.

6. The compound (C) of formulae (II) according to claim 1, or the N-oxide, pharmaceutically acceptable salt, pharmaceutically acceptable solvate, or stereoisomer thereof, wherein said compound is of formula (II-a-1) or (II-b-1) or (II-c-1) [compounds (C) of class (II) hereinafter]: 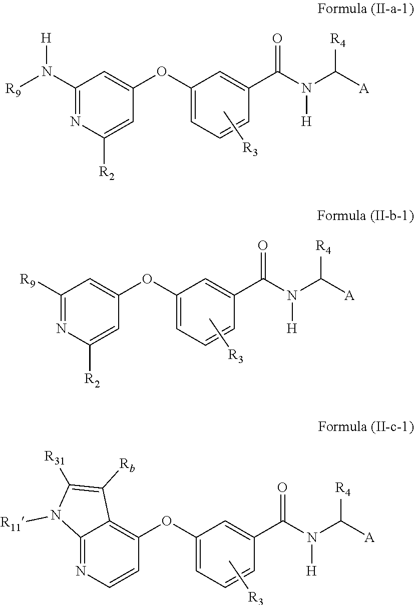
wherein A, R₄, R₃, R₂, and R₉ have the same meaning as defined above for formula (II); wherein R₃₁ is a heteroaryl which is optionally substituted with a C₁₋₄ alkyl, wherein R₁₁′ is hydrogen or C₁₋₄ alkyl; and wherein R_{b} is selected from the group consisting of hydrogen, halo, C₁₋₄ alkyl, and C₁₋₆ cycloalkyl; and
wherein said heteroaryl is a monocyclic ring structure containing 5 or 6 ring atoms, or a bicyclic aromatic group having 8 to 10 atoms, containing 1-3 heteroatoms independently selected from O or N.

7. The compound (C) of formulae (IV) or (VI) according to claim 1, or the N-oxide, pharmaceutically acceptable salt, pharmaceutically acceptable solvate, or stereoisomer thereof, wherein said compound is of formula (IV-a-1) to (IV-c-1) [compounds (C) of class (IV) hereinafter] or of formula (VI-a-1) to (VI-c-1) [compounds (C) of class (VI) hereinafter]: 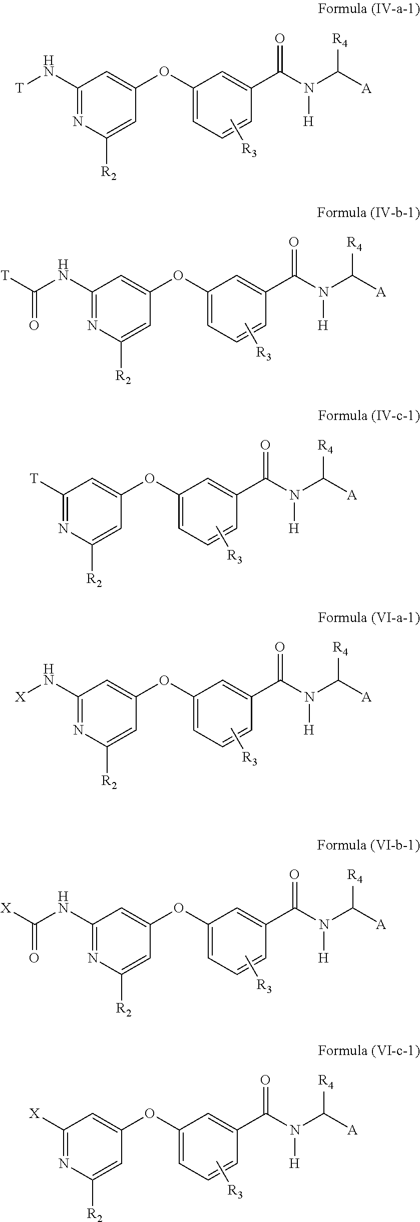
wherein A, R₄, R₃, R₂, T, and X are as defined as in claim 1.

8. The compound (C) of formula (VII) according to claim 1, or the N-oxide, pharmaceutically acceptable salt, pharmaceutically acceptable solvate, or stereoisomer thereof, wherein said compound is of formula (VII-a-1) or (VII-b-1) [compounds (C) of class (VII) hereinafter]: 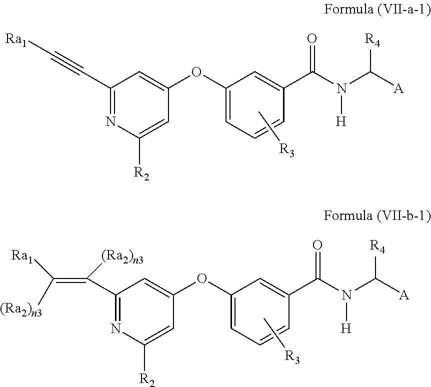
wherein A, R₄, R₃, R₂, Rₐ₁, Rₐ₂, and n3 are as defined as in claim 1.

9. The compound (C) of formula (II) according to claim 1, or the N-oxide, pharmaceutically acceptable salt, pharmaceutically acceptable solvate, or stereoisomer thereof, wherein said compound is of formula (II-a-2), or (II-b-2), or (II-c-2) [compounds (C) of class (II) hereinafter]: 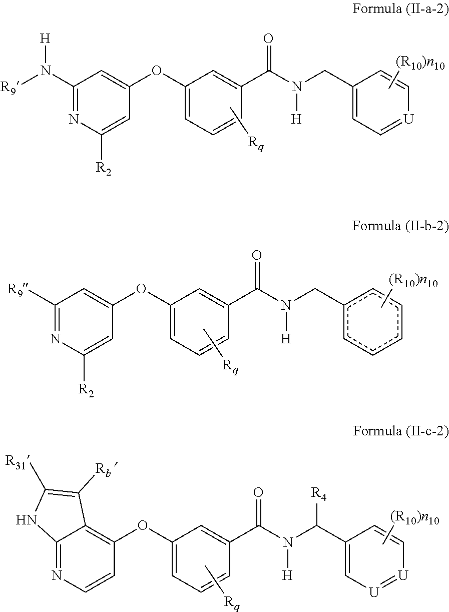
wherein:
each of R₉′ is selected from the group consisting of hydrogen, CN and C₃₋₆ cycloalkyl such as cyclopropyl;
each of R₉″ is selected from the group consisting of hydrogen, C₁₋₄ alkyl, CN and C₃₋₆ cycloalkyl such as cyclopropyl;
each of R₂ is independently selected from hydrogen or halo;
each of R₉ is independently selected from the group consisting of hydrogen, CH₃, OCH₃, and halo, such as F or Cl;
each of R₁₀ is independently selected from the group consisting of H, F, Cl, OCH₃, or CF₃;
each of U is selected from the group consisting of C, C—R₁₀ and N;
n₁₀ is an integer equal to 0, 1 or 2;
each of R₃₁′ is selected from the group consisting of pyrazyl, N-methylpyrazyl, and pyridyl;
R_{b}′ is selected from the group consisting of hydrogen, halo, C₁₋₄ alkyl, and C₁₋₄ cycloalkyl; preferably R_{b}′ is selected from the group consisting of C1, CH₃, and cyclopropyl; and
the dash bond represents an optional double bond.

10. The compound (C) of formula (IV) according to claim 1, or the N-oxide, pharmaceutically acceptable salt, pharmaceutically acceptable solvate, or stereoisomer thereof, wherein said compound is of formula (IV-a-2-1), (IV-a-2-2), (IV-b-2-1), (IV-b-2-2), or (IV-c-2-1) to (IV-c-2-4) [compounds (C) of class (IV) hereinafter]: 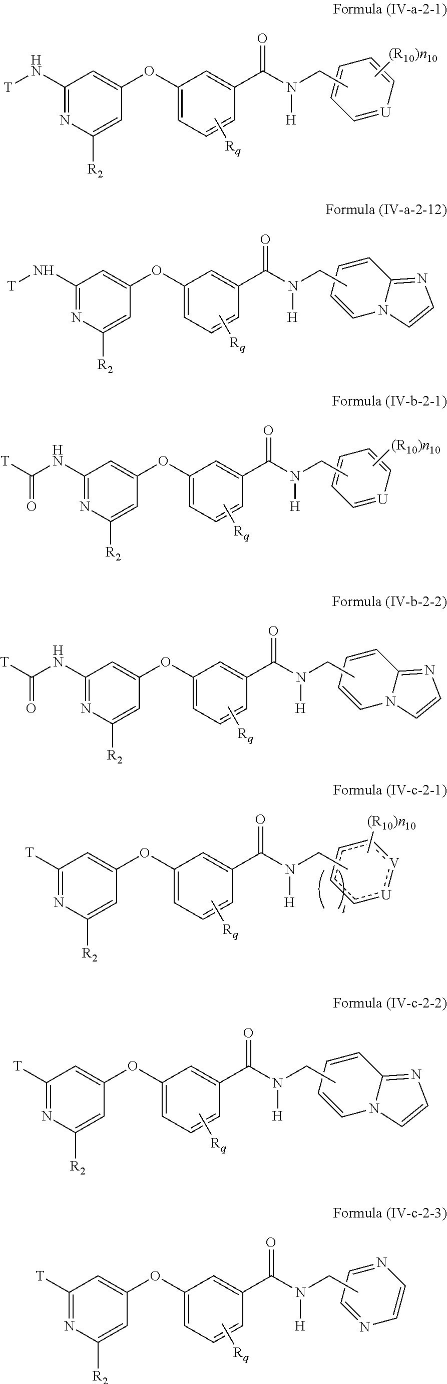 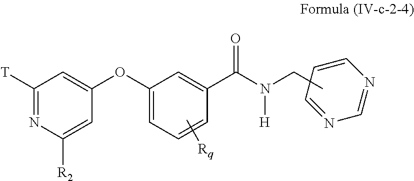
wherein:
T is, independently from each other and at each occurrence, selected from the moiety of formula (T-a-a) to (T-a-f) herein below: 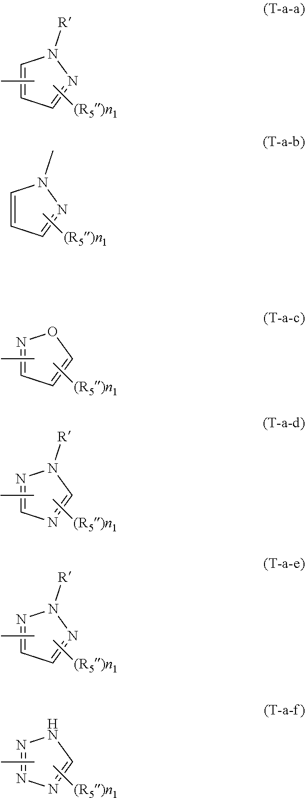
wherein:
each of R′ is independently hydrogen, C₁₋₄ alkyl, cycloalkyl selected from the group consisting of cyclopropyl and cyclobutyl; heterocyclyl selected from the group consisting of oxetanyl, tetrahydropyranyl, azetdinyl, and piperidinyl; wherein said alkyl is further optionally substituted with F, OC₁₋₄ alkyl, P(═O)(OC₁₋₄alkyl)₂, P(═O)(C₁₋₄alkyl)₂, CN, cyclopropyl, or cyclobutyl; and wherein said heterocyclyl is further optionally substituted with C(═O)(OC₁₋₄alkyl),
each of R″s is independently selected from the group consisting of hydrogen, C₁₋₄ alkyl, CF₃ and cyclopropyl;
each of n1, independently from each other and at each occurrence is an integer equal to 0, 1 or 2;
R₂ is independently hydrogen, halo, or NH₂;
each of R₉ is independently selected from the group consisting of H, CH₃, OCH₃, and halo, such as F or Cl;
each of R₁₀ is independently selected from the group consisting of hydrogen, halo, C₁₋₄ alkyl, CF₃, OC₁₋₄alkyl, CN;
each of U and V are independently C, C—R₁₀ or N; and
n₁₀ is an integer equal to 0, 1 or 2.

11. The compound (C) of formula (VI) according to claim 1, or the N-oxide, pharmaceutically acceptable salt, pharmaceutically acceptable solvate, or stereoisomer thereof, wherein said compound is of formula (VI-a-2) to (VI-c-2) [compounds (C) of class (VI) hereinafter]: 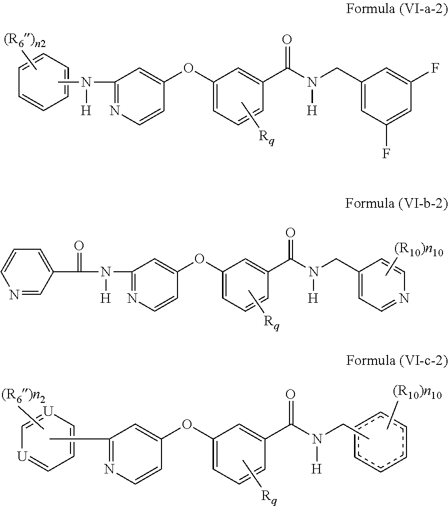
wherein
each of R″6 is independently selected from the group consisting of hydrogen, halo, C₁₋₄ alkyl, N(R₂₁)₂, OR₂₁; heterocyclyl selected from the group consisting of pyrrolidyl, piperidyl, morpholinyl, piperazyl and a pyrazyl;
wherein said heterocyclyl and pyrazyl are optionally substituted with C₁₋₄ alkyl, and wherein R₂₁ is a C₁₋₄ alkyl;
each of R₉ is independently selected from the group consisting of H, CH₃, OCH₃, and halo, such as F or Cl;
each of R₁₀ is independently selected from the group consisting of hydrogen, halo, OC-4 alkyl, and CN;
each of U is independently C, C—R₁₀ or N;
n₁₀ is an integer equal to 0, 1 or 2; and
n₂ is an integer equal to 0, 1 or 2.

12. The compound (C) of formula (VII) according to claim 1, or the N-oxide, pharmaceutically acceptable salt, pharmaceutically acceptable solvate, or stereoisomer thereof, wherein said compound is of formula (VII-a-2) [compounds (C) of class (VI) hereinafter]: 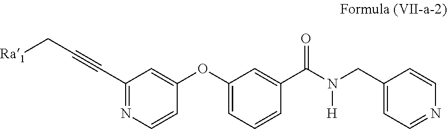
wherein Rₐ′ is selected from the group consisting of benzyl, pyrazyl, OH, OC₁₋₄ alkyl, NH₂, and NH(C₁₋₄ alkyl) and wherein R₉ is selected from the group consisting of H, CH₃, OCH₃, and halo, such as F or Cl; preferably R_{q} is H or CH₃.

13. The compound (C) of formulae (IV-a-1), (IV-b-1), or (IV-c-1), (VI-a-1), (VI-b-1) or (VI-c-2) according to claim 7, or the N-oxide, pharmaceutically acceptable salt, pharmaceutically acceptable solvate, or stereoisomer thereof, wherein compound of formulae (IV-a-1), (IV-b-1), (IV-c-1), (VI-a-1), (VI-b-1) or (VI-c-2) is a compound according to formula (XXXVII) to (CCLX) herein below:

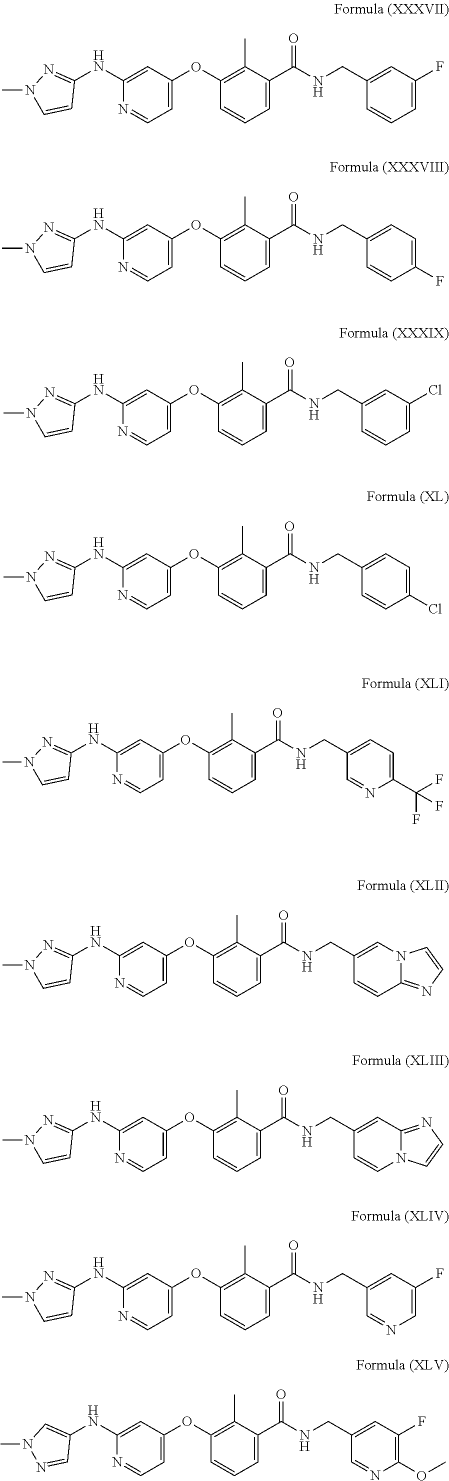

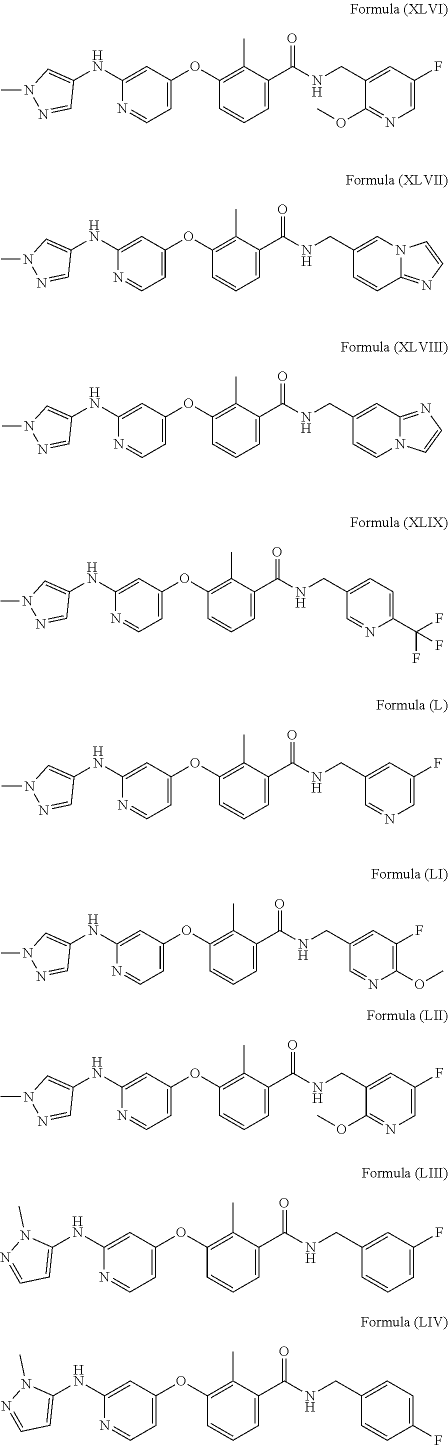

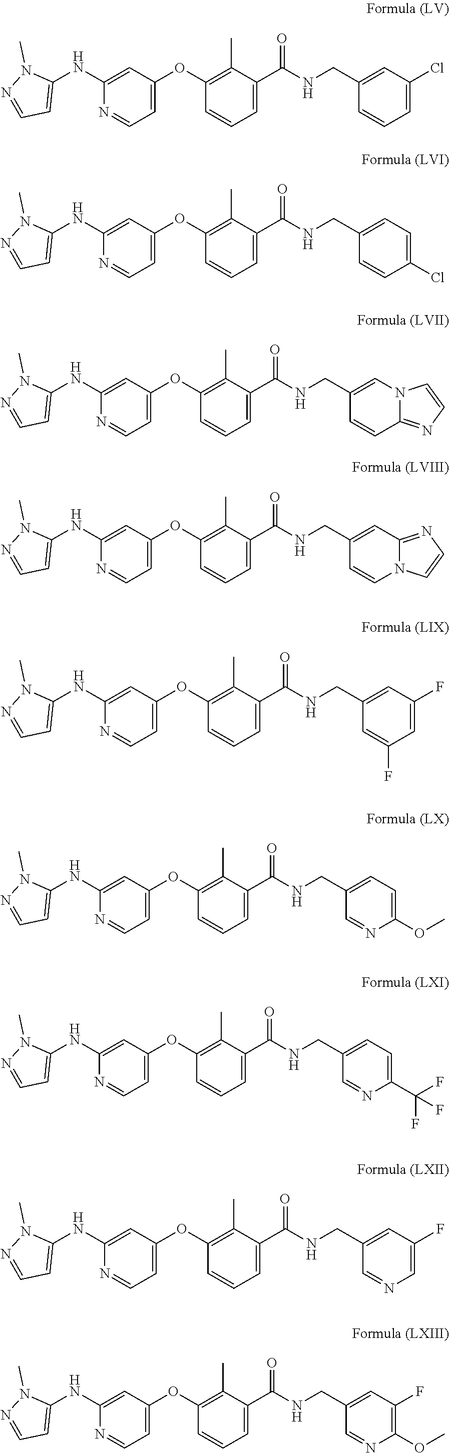

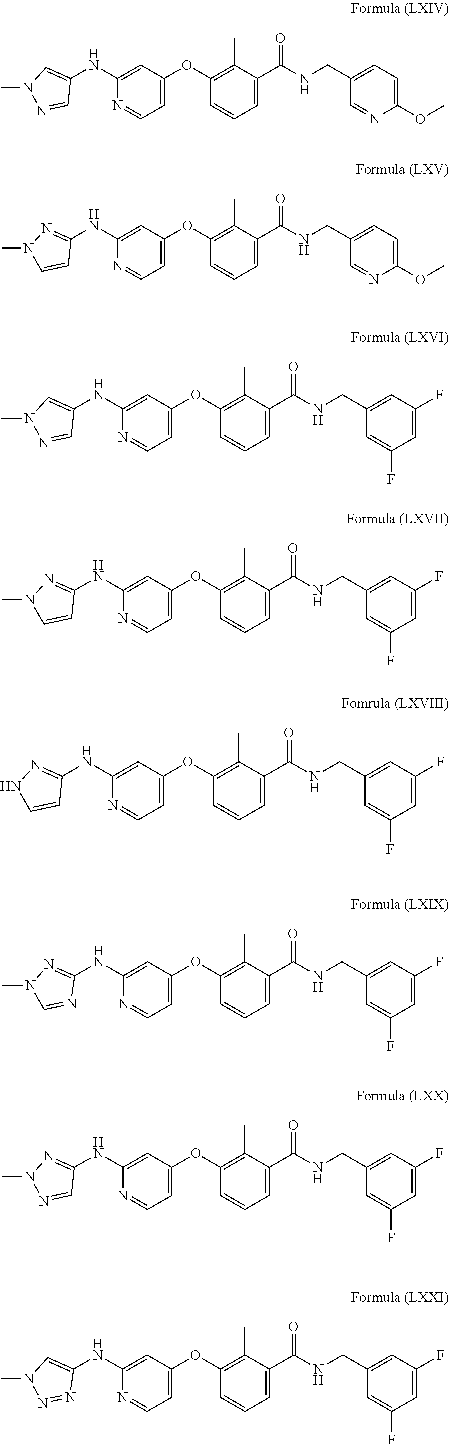

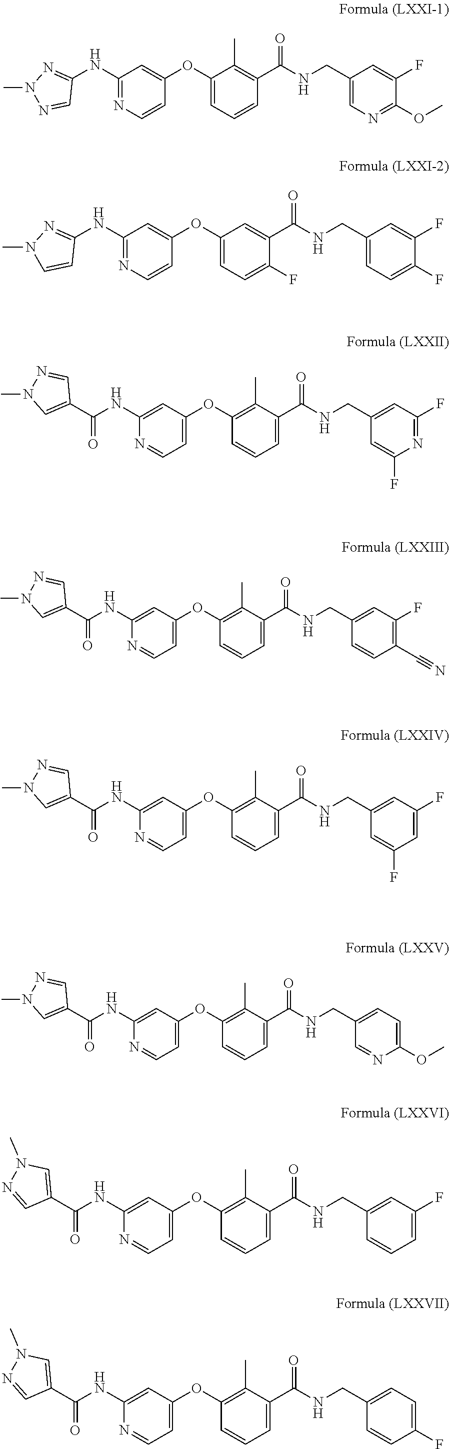

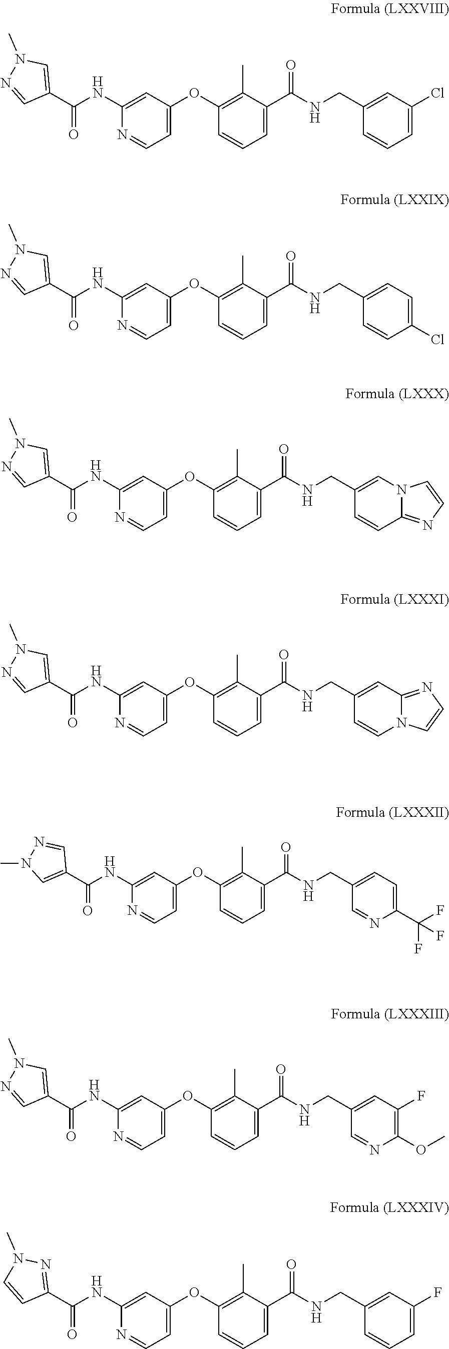

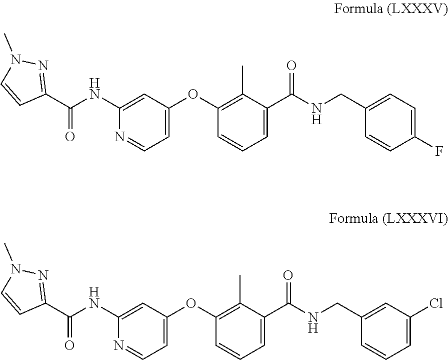 [SMILES: Cc1c(Oc2ccnc(NC(=O)c3ccn(C)n3)c2)cccc1C(=O)NCc1ccc(F)cc1 ; Cc1c(Oc2ccnc(NC(=O)c3ccn(C)n3)c2)cccc1C(=O)NCc1cccc(Cl)c1]

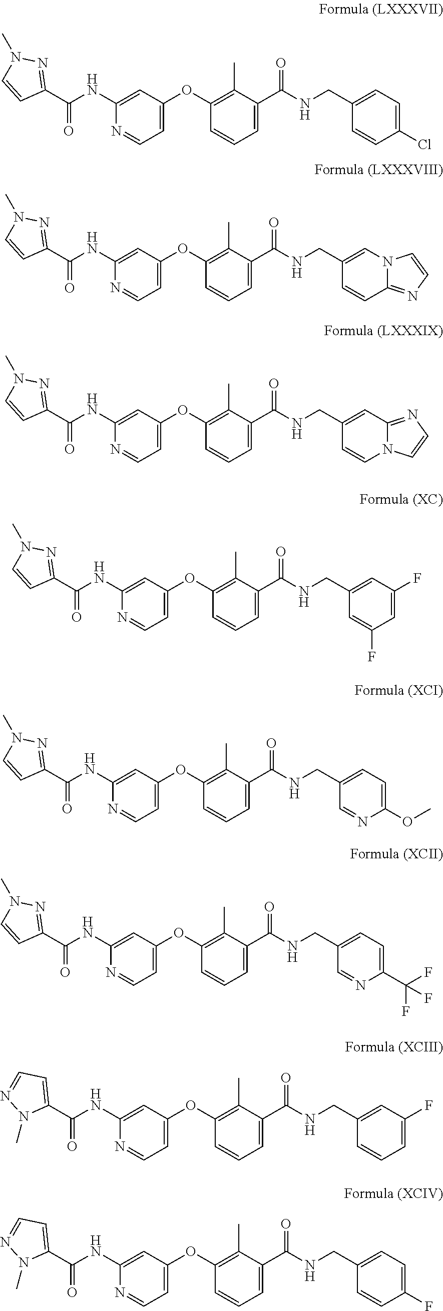

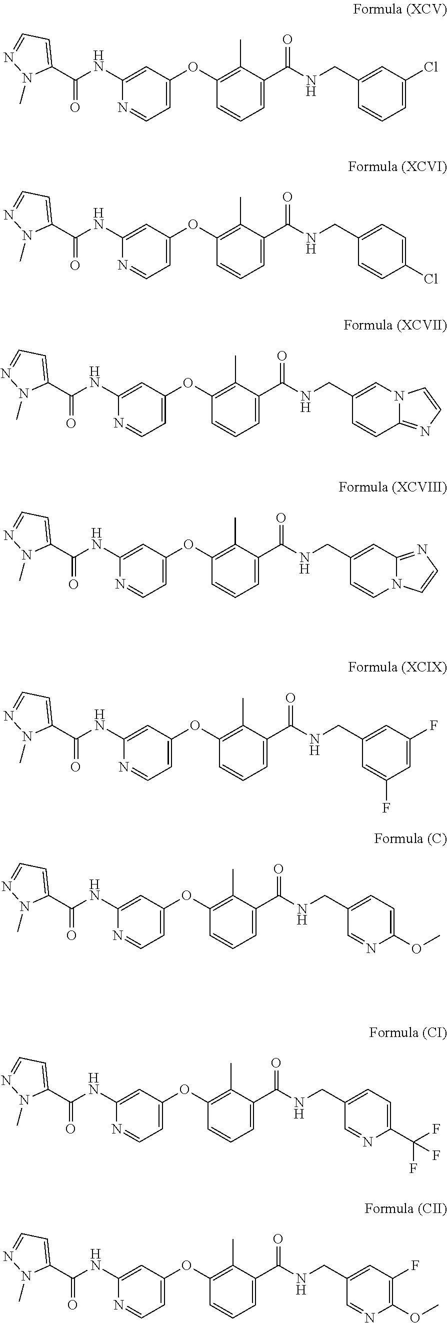

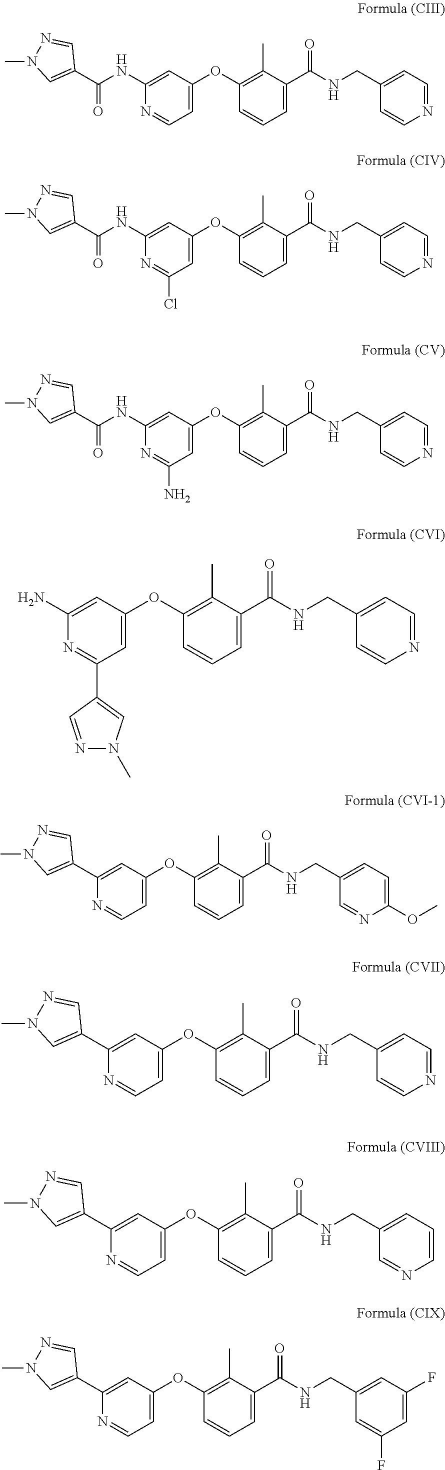

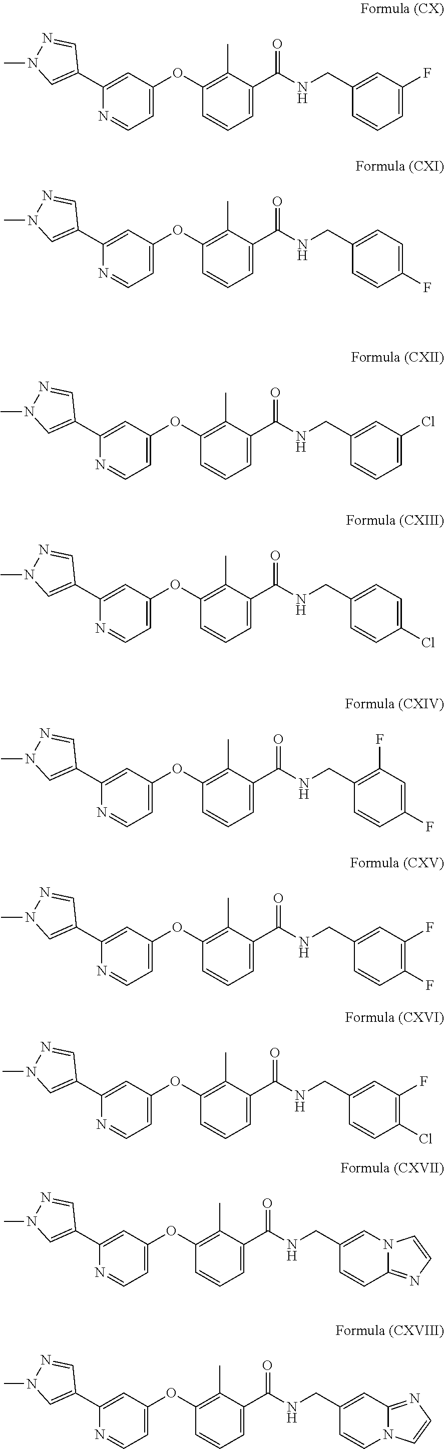

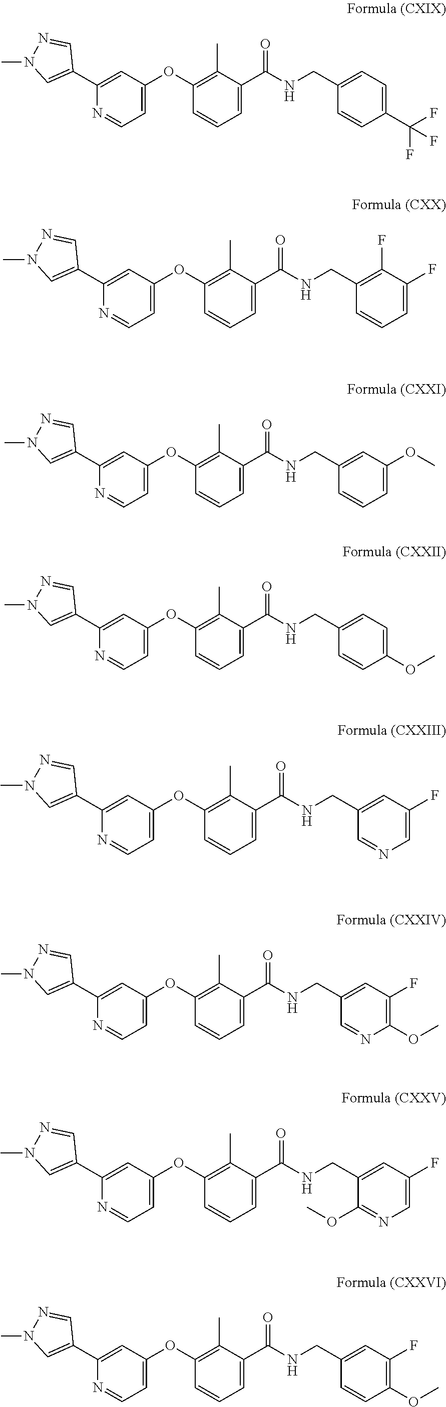

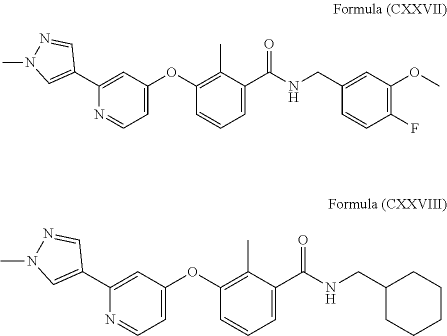

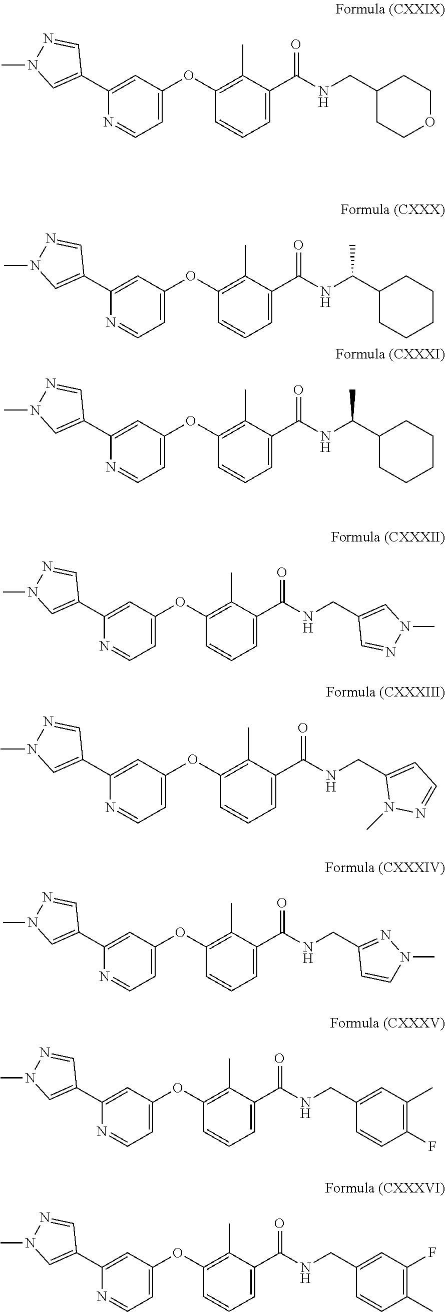

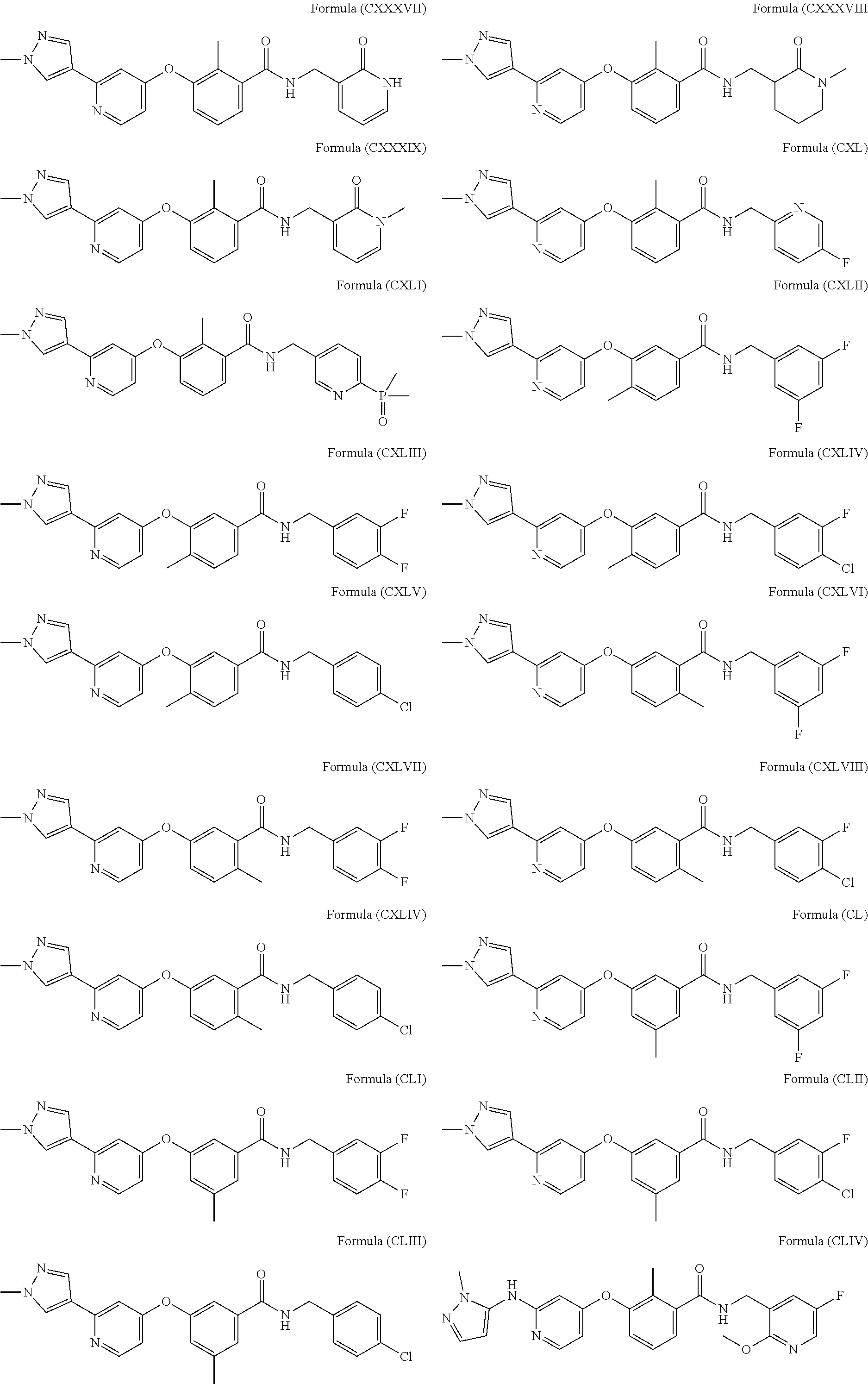

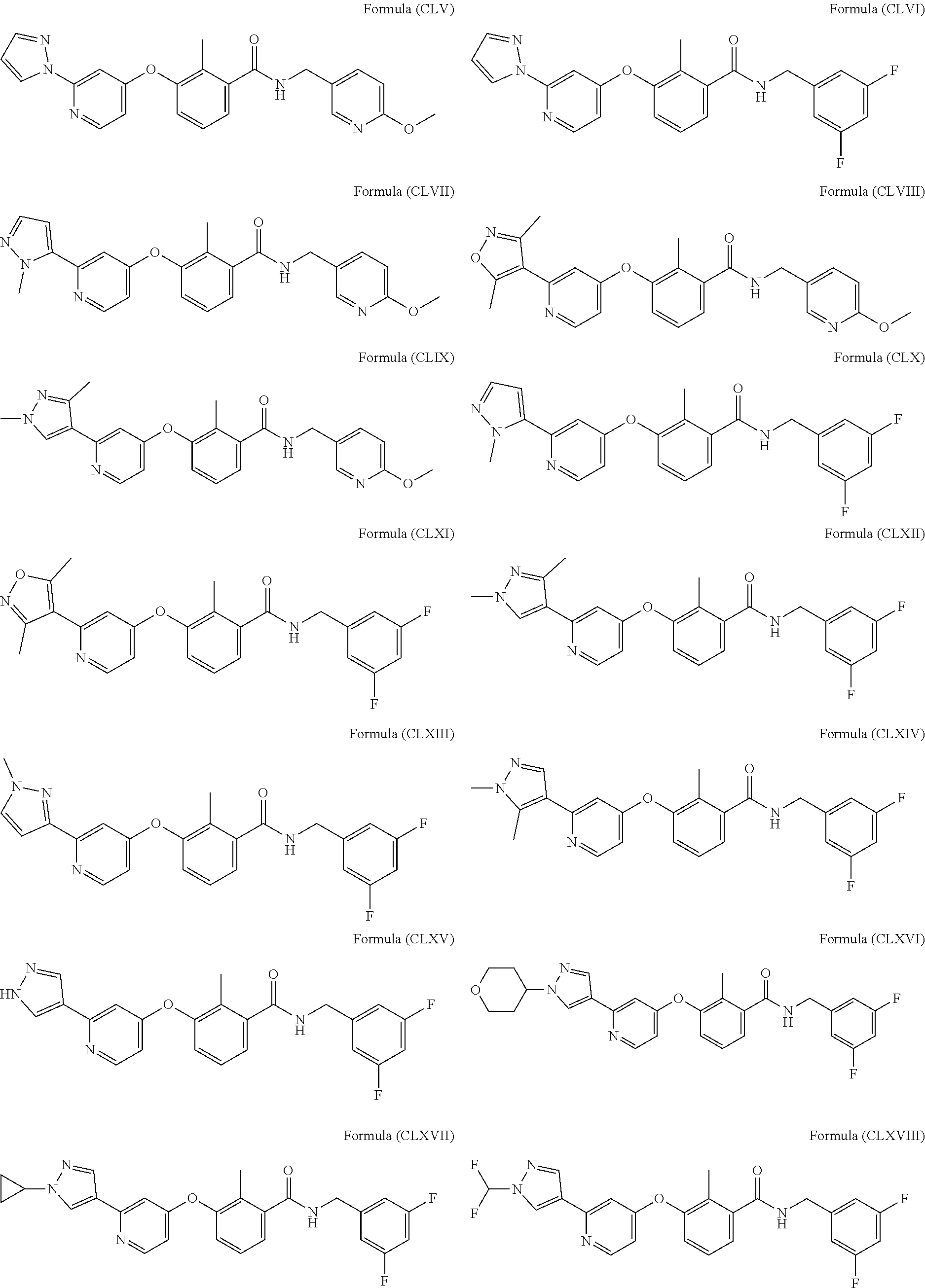

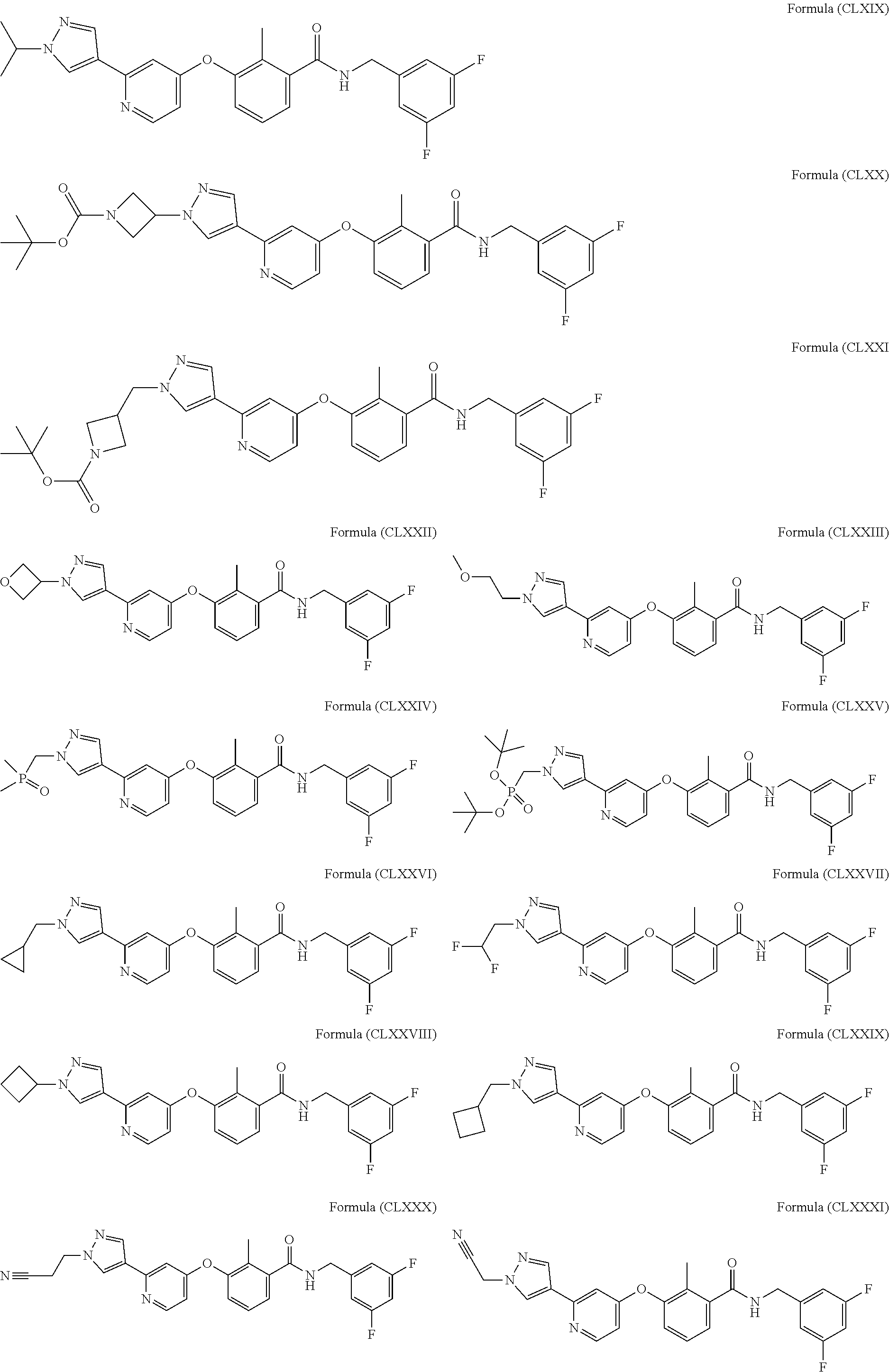

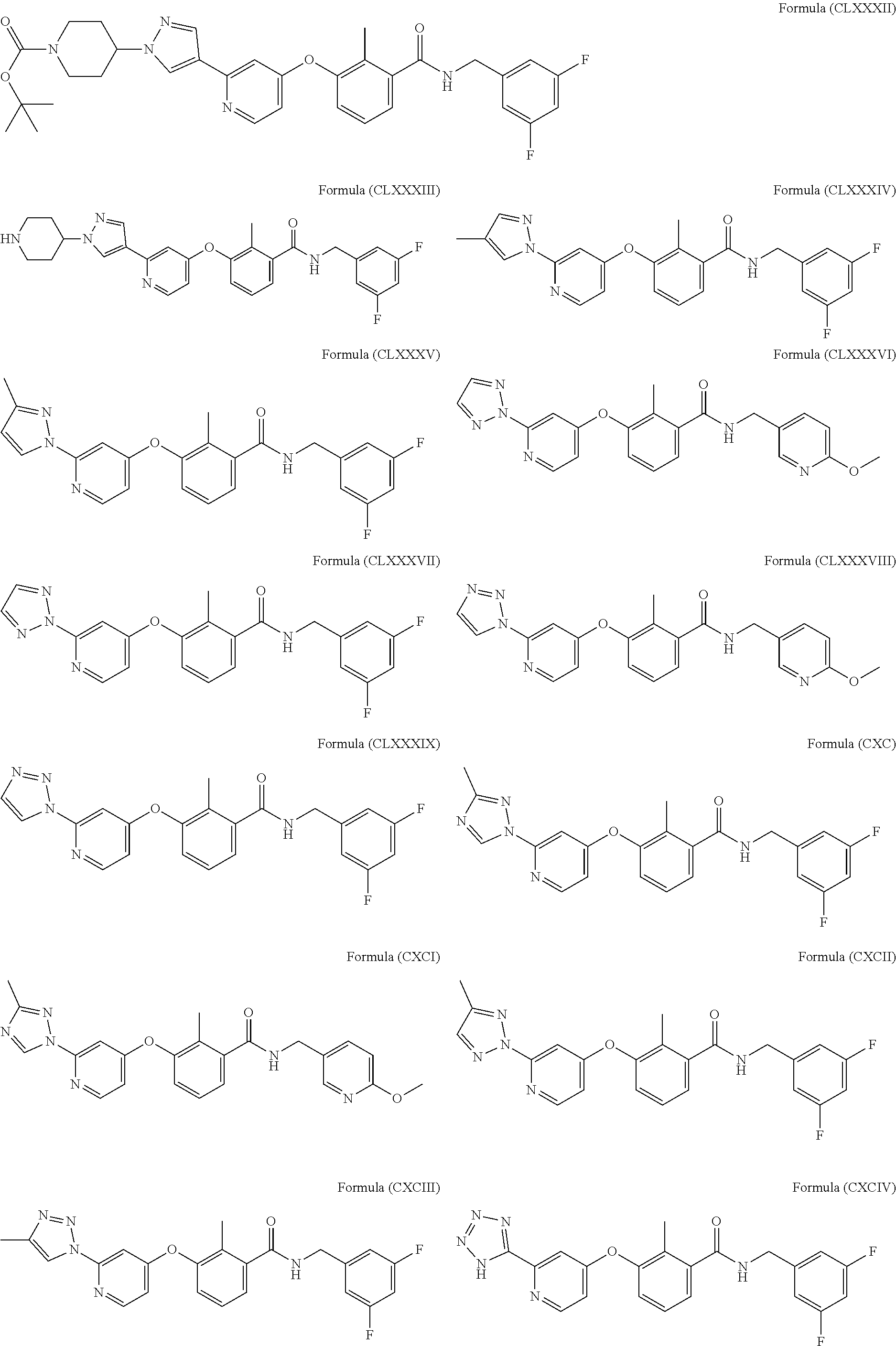

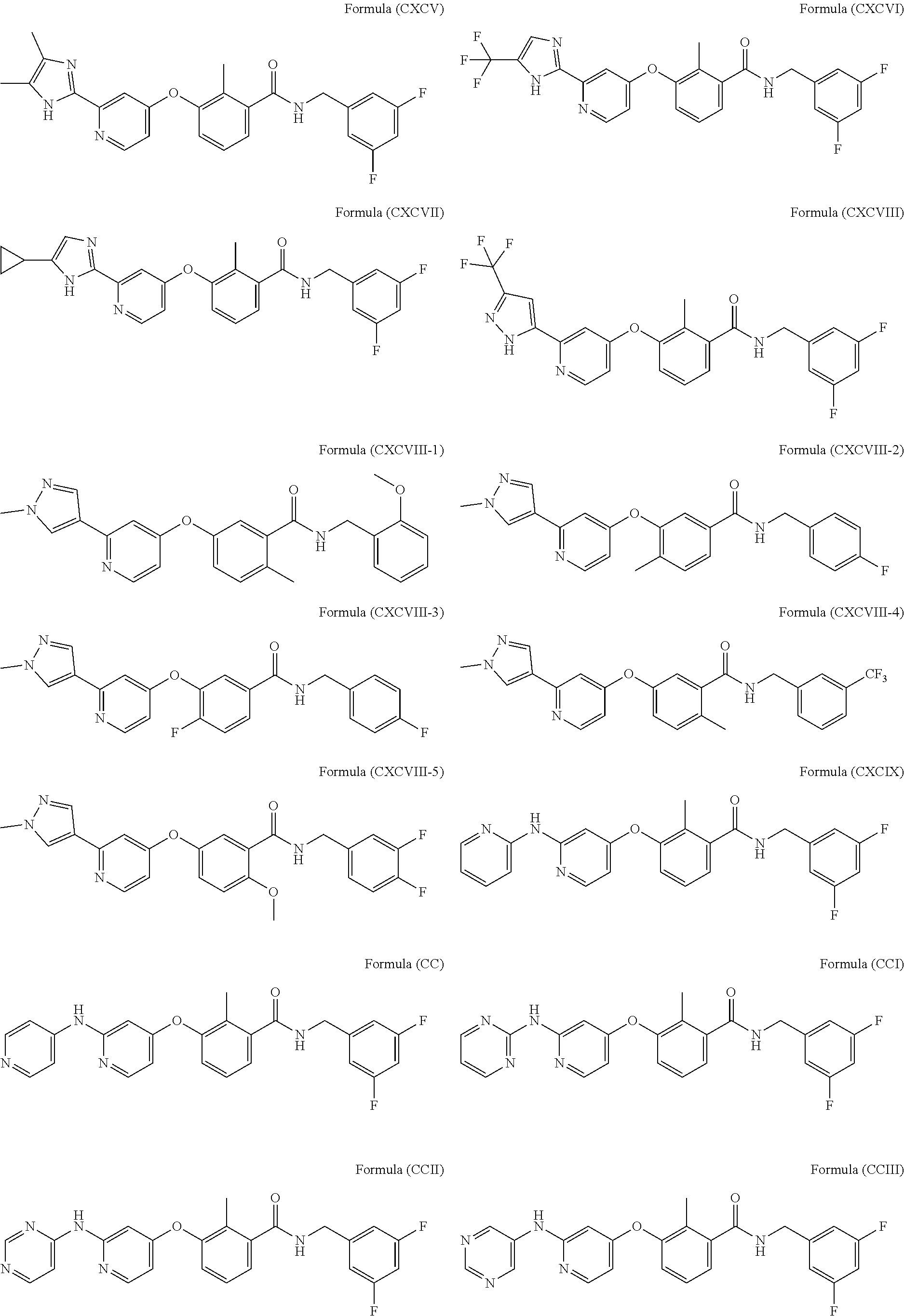

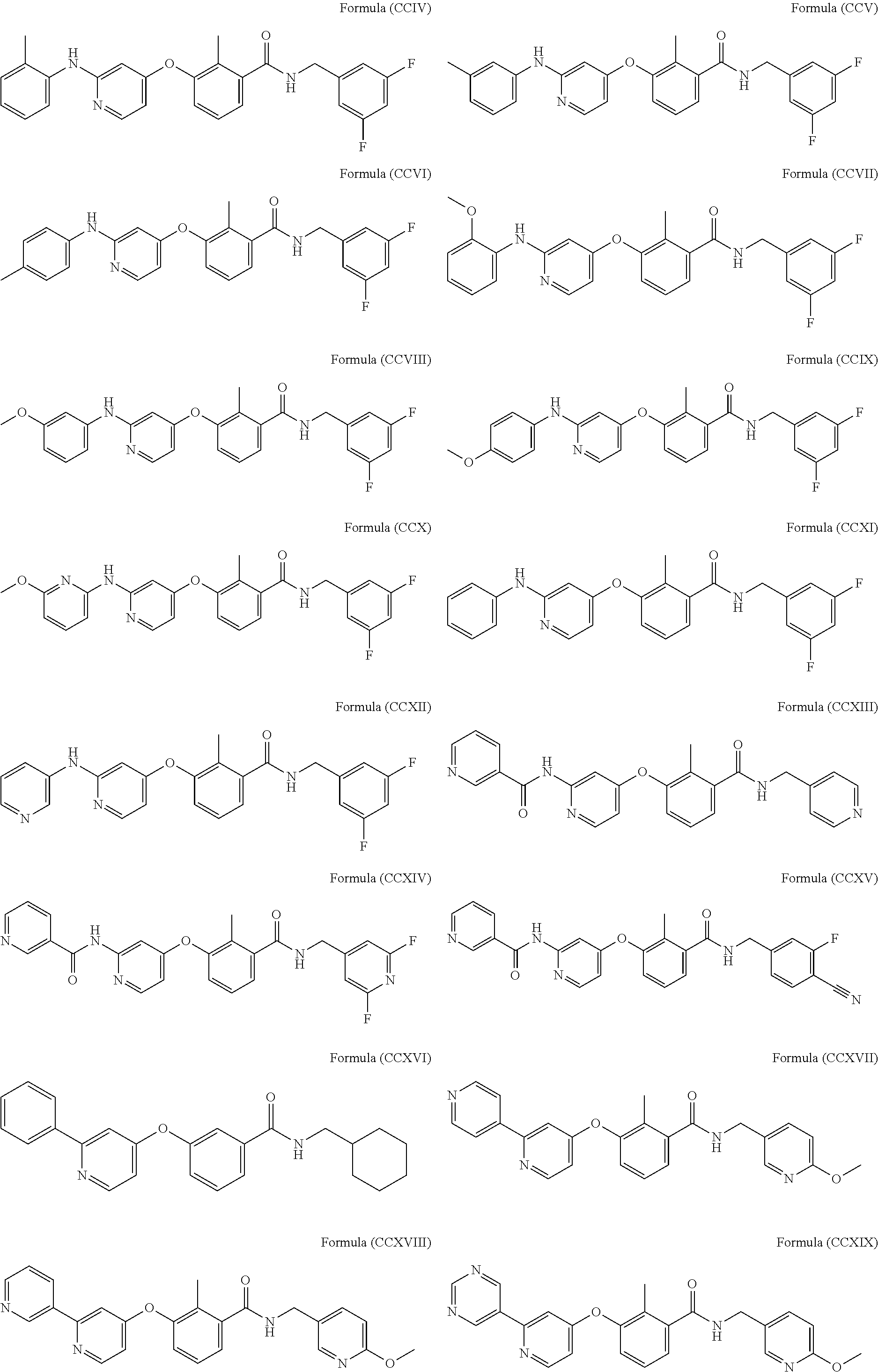

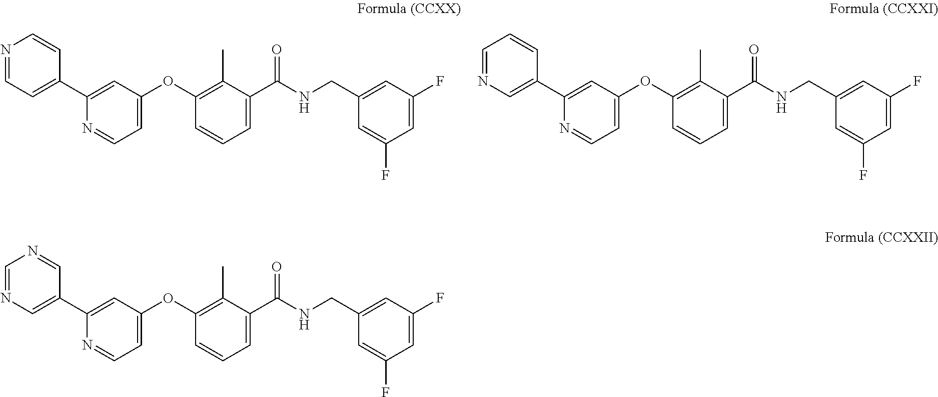

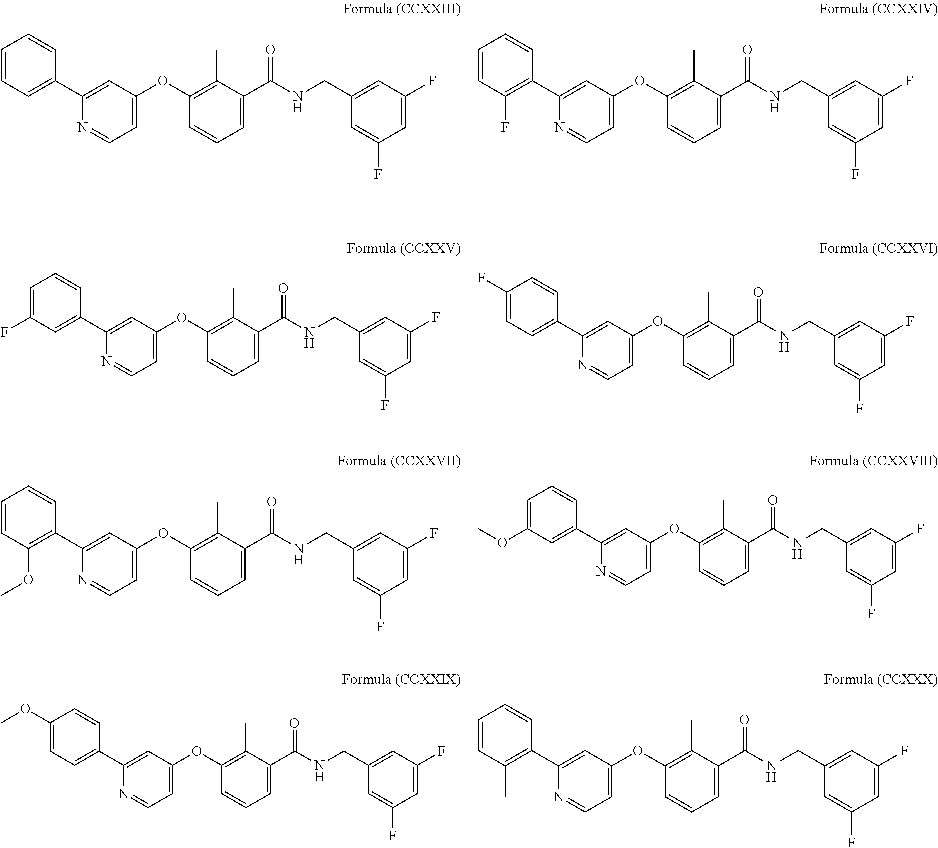

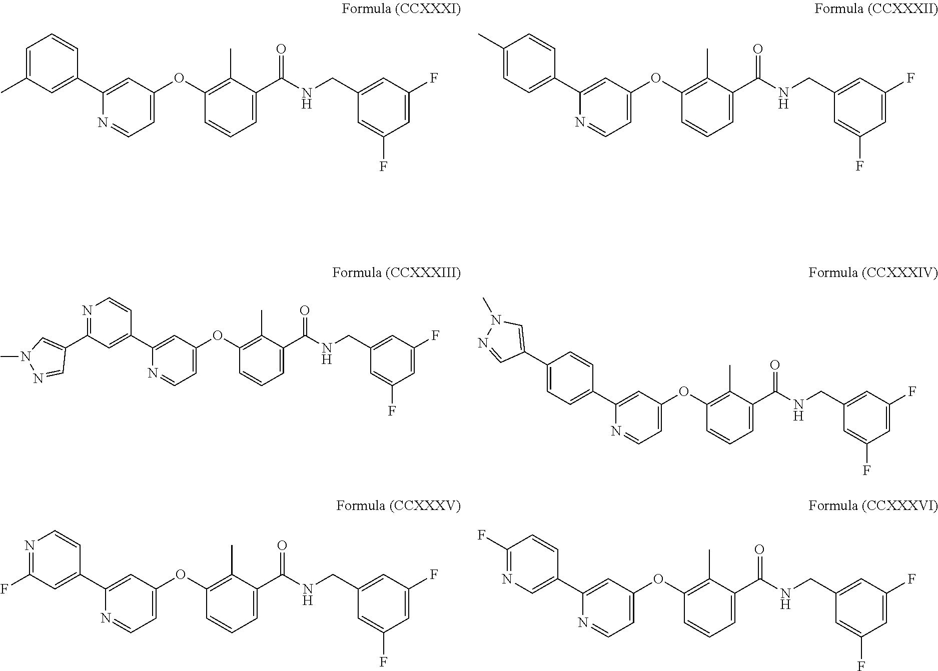 [SMILES: Cc1cccc(-c2cc(Oc3cccc(C(=O)NCc4cc(F)cc(F)c4)c3C)ccn2)c1 ; Cc1ccc(-c2cc(Oc3cccc(C(=O)NCc4cc(F)cc(F)c4)c3C)ccn2)cc1 ; Cc1c(Oc2ccnc(-c3ccnc(-c4cnn(C)c4)c3)c2)cccc1C(=O)NCc1cc(F)cc(F)c1 ; Cc1c(Oc2ccnc(-c3ccc(-c4cnn(C)c4)cc3)c2)cccc1C(=O)NCc1cc(F)cc(F)c1 ; Cc1c(Oc2ccnc(-c3ccnc(F)c3)c2)cccc1C(=O)NCc1cc(F)cc(F)c1 ; Cc1c(Oc2ccnc(-c3ccc(F)nc3)c2)cccc1C(=O)NCc1cc(F)cc(F)c1]

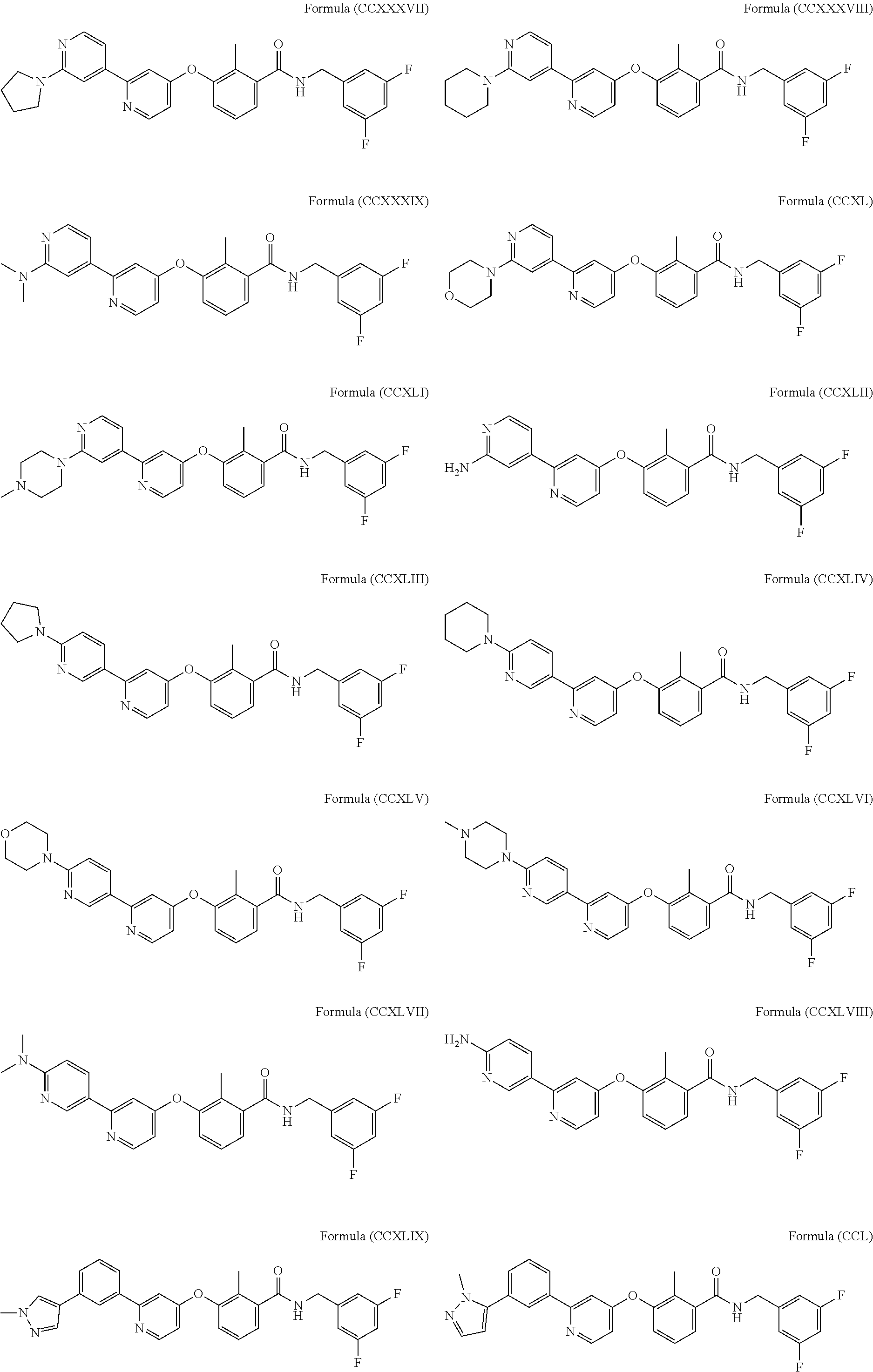

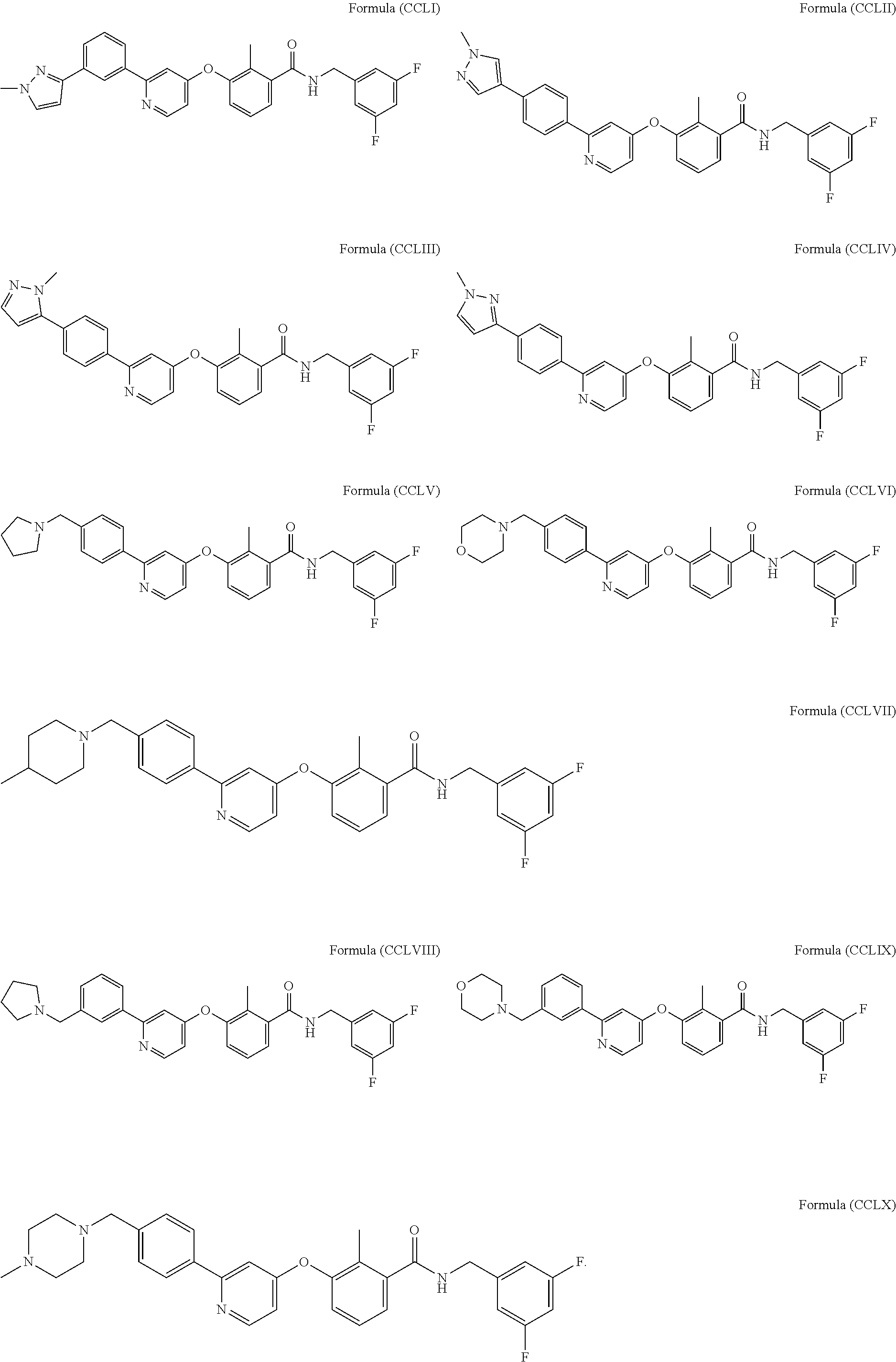

14. A pharmaceutical composition comprising a carrier, and as active ingredient the compound (C), as defined according to claim 1.

15. A compound (C) as defined according to claim 1, for use as a medicament.
